(19) United States
(12) Patent Application Publication
(10) Pub. No.: US 2024/0293543 A1
(43) Pub. Date: Sep. 5, 2024

(54) ENGINEERED CELLS FOR THERAPY

(71) Applicant: Editas Medicine, Inc., Cambridge, MA (US)

(72) Inventor: John Anthony Zuris, Boston, MA (US)

(21) Appl. No.: 18/573,231

(22) PCT Filed: Jun. 23, 2022
(86) PCT No.: PCT/US22/73126

§ 371 Date: Dec. 21, 2023

Related U.S. Application Data
(60) Provisional application No. 63/340,225, filed on May 10, 2022.
(60) Provisional application No. 63/233,695, filed on Aug. 16, 2021.
(60) Provisional application No. 63/214,157, filed on Jun. 23, 2021.

(51) Int. Cl. A61K 39/00; C07K 14/74; C12N 5/0783; C12N 9/02; C12N 9/22; C12N 15/11; C12N 15/90

(52) U.S. Cl. CPC A61K 39/4613; A61K 39/4634; C07K 14/70539; C12N 5/0646; C12N 9/0008; C12N 9/22; C12N 15/11; C12N 15/907; C12Y 102/01012; A61K 2239/26; C12N 2310/20; C12N 2501/16; C12N 2506/45; C12N 2800/80

(57) ABSTRACT

Edited cells, e.g., genomically edited cells, with reduced levels of immune rejection and/or improved persistence are described.

### CROSS REFERENCE TO RELATED APPLICATIONS

This application claims the benefit of U.S. Provisional Applications Nos. 63/214,157 filed Jun. 23, 2021, 63/233,695 filed Aug. 16, 2021, and 63/340,225 filed May 10, 2022. The entirety of each of the priority applications is incorporated herein by reference.

### BACKGROUND

There remains a need for engineered cells for therapeutic interventions, such as engineered embryonic stem cells and/or engineered induced pluripotent cells, and/or progeny of, or cells differentiated from, such engineered cells (e.g., iNK cells), with a reduced level of immune rejection and/or improved persistence.

### SUMMARY

Some aspects of the present disclosure are based, at least in part, on methods and systems for genetically modifying NK cells and/or pluripotent stem cells (e.g., iPSCs) that are, e.g., differentiated into modified iNK cells, to include one or more gain-of-function modifications (e.g., one or more gain-of-function modifications described herein), and to include one or more loss-of-function modifications (e.g., one or more loss-of-function modifications described herein), as well as modified NK cells and/or modified pluripotent stem cells (e.g., iPSCs) that are, e.g., differentiated into modified iNK cells (and compositions of such cells) that include one or more gain-of-function modifications (e.g., one or more gain-of-function modifications described herein), and that include one or more loss-of-function modifications (e.g., one or more loss-of-function modifications described herein). In certain aspects of the disclosure, such modified NK cells and/or modified pluripotent stem cells (e.g., iPSCs) that are, e.g., differentiated into modified iNK cells, include at least one gain-of-function modification within a coding region of an essential gene (e.g., an essential gene described herein).

In one aspect, the disclosure features a pluripotent stem cell (e.g., an iPSC cell), a primary cell (e.g., a Natural Killer (NK) cell), an iNK cell, a progeny or daughter cell of such cell, or a population of such cells, wherein the cell comprises: (i) a genomic edit that results in loss of function of Beta-2-Microglobulin (B2M), and (ii) a genome comprising an exogenous nucleic acid comprising a nucleotide sequence encoding an HLA-E polypeptide. In some embodiments, the exogenous nucleic acid comprises a nucleotide sequence encoding a portion of a B2M polypeptide. In some embodiments, the exogenous nucleic acid comprises a nucleotide sequence encoding peptide (e.g., an HLA-G signal peptide). In some embodiments, the peptide comprises the amino acid sequence of RIIPRHLQL (SEQ ID NO: 1234), VMAPRTLFL (SEQ ID NO: 1235), VMAPRTLIL (SEQ ID NO: 1236), VMAPRTVLL (SEQ ID NO: 1237), and/or VMAPRTLVL (SEQ ID NO: 1238). In some embodiments, the exogenous nucleic acid comprises, from 5′ to 3′, the nucleotide sequence encoding the peptide (e.g., HLA-G signal peptide), the nucleotide sequence encoding the portion of the B2M polypeptide, and the nucleotide sequence encoding the HLA-E polypeptide. In some embodiments, the exogenous nucleic acid comprises a first linker sequence between the nucleotide sequence encoding the peptide (e.g., the HLA-G signal peptide) and the nucleotide sequence encoding the portion of the B2M polypeptide, and a second linker sequence between the nucleotide sequence encoding the portion of the B2M polypeptide and the nucleotide sequence encoding the HLA-E polypeptide.

In some embodiments, the exogenous nucleic acid consists of or comprises the nucleotide sequence of SEQ ID NO: 1181 or 1230. In some embodiments, the exogenous nucleic acid encodes a polypeptide that consists of or comprises the amino acid sequence of SEQ ID NO: 1182, 1231, 1243, 1244, 1245, or 1246.

In some embodiments, the cell comprises a genomic edit that results in a loss of function of an agonist of the TGF beta signaling pathway, a genomic edit that results in loss of function of Cytokine Inducible SH2 Containing Protein (CISH), a genomic edit that results in loss of function of class II, major histocompatibility complex, transactivator (CIITA), and/or a genomic edit that results in a loss of function of adenosine A2a receptor (ADORA2A).

In some embodiments, the exogenous nucleic acid is in frame with and downstream (3′) of an exogenous coding sequence or partial coding sequence of an essential gene. In some embodiments, the essential gene is a housekeeping gene, e.g., a gene listed in Table 13. In some embodiments, the essential gene encodes glyceraldehyde 3-phosphate dehydrogenase (GAPDH).

In some embodiments, the genome comprising the exogenous nucleic acid is produced by contacting a pluripotent stem cell with (i) a nuclease that causes a break within the endogenous coding sequence of the essential gene, and (ii) a donor template that comprises a knock-in cassette comprising the exogenous nucleic acid in frame with and downstream (3′) of an exogenous coding sequence or partial coding sequence of the essential gene, wherein the knock-in cassette is integrated into the genome of the cell by homology-directed repair (HDR) of the break.

In some embodiments, the cell is an induced pluripotent stem cell (iPSC). In some embodiments, the cell is a daughter cell of the iPSC. In some embodiments, the cell is a differentiated cell from the iPSC. In some embodiments, the differentiated cell is an immune cell. In some embodiments, the differentiated cell is a lymphocyte. In some embodiments, the differentiated cell is an induced Natural Killer (INK) cell. In some embodiments, the cell is a progeny or daughter cell of such differentiated cell (e.g., an iNK cell).

In some embodiments, the cell or differentiated cell is for use as a medicament. In some embodiments, the cell or differentiated cell is for use in the treatment of a disease, disorder, or condition, e.g., a tumor and/or a cancer.

In some embodiments, the population of cells comprises such pluripotent stem cell, differentiated cell, or progeny or daughter cell.

In some embodiments, the population of cells comprises an iNK cell described herein (e.g., comprising: (i) the genomic edit that results in loss of function of Beta-2-Microglobulin (B2M), and (ii) the genome comprising the exogenous nucleic acid comprising a nucleotide sequence encoding an HLA-E polypeptide). In some embodiments, the population of cells is characterized in that, when contacted with natural killer (NK) cells, a level of activation of NK cells is decreased (e.g., by at least about 10%, 20%, 40%, 60%, 80%, or 100%), relative to a reference level of activation of NK cells when contacted with a reference population of cells (as determined using, e.g., a method described herein). In some embodiments, the population of cells is characterized in that, when contacted with NK cells, a level of degranulation of NK cells is decreased (e.g., by at least about 10%, 20%, 40%, 60%, 80%, or 100%) relative to a reference level of degranulation of NK cells when contacted with a reference population of cells (as determined using, e.g., a method described herein). In some embodiments, the population of cells is characterized in that, when contacted with NK cells, a level of cell death and/or lysis of the population of cells is decreased (e.g., by at least about 10%, 20%, 40%, 60%, 80%, or 100%) relative to a reference level of cell death and/or lysis of a reference population of cells when contacted with NK cells (as determined using, e.g., a method described herein). In some embodiments, the NK cells are human donor NK cells and/or peripheral blood NK cells.

In some embodiments, the reference population of cells does not comprise iNK cells comprising a genome comprising the exogenous nucleic acid. In some embodiments, the reference population of cells does not comprise iNK cells comprising the genomic edit that results in loss of function of B2M. In some embodiments, the reference population of cells comprises iNK cells that are the same as the population of genomically edited iNK cells, but whose genomes do not comprise the exogenous nucleic acid (e.g., encoding the HLA-E polypeptide) and whose genomes do not comprise the genomic edit that results in loss of function of B2M.

In another aspect, the disclosure features a composition, e.g., a pharmaceutical composition, comprising a pluripotent stem cell, differentiated cell, progeny or daughter cell, or population of cells described herein. In some embodiments, the pharmaceutical composition comprises a pharmaceutically acceptable carrier.

In another aspect, the disclosure features a method of treating a condition, disorder, and/or disease, comprising administering to a subject suffering therefrom a pluripotent stem cell, differentiated cell, progeny or daughter cell, or population of cells described herein, or a pharmaceutical composition described herein. In some embodiments, the subject is suffering from a tumor, e.g., a solid tumor. In some embodiments, the subject is suffering from a cancer. In some embodiments, the pluripotent stem cell, the differentiated cell, the progeny or daughter cell, or the population of cells is allogeneic to the subject. In some embodiments, the subject is a human.

In another aspect, the disclosure features a method, comprising administering to a subject a pluripotent stem cell, differentiated cell, progeny or daughter cell, or population of cells described herein, or a pharmaceutical composition described herein. In some embodiments, the subject is suffering from a tumor, e.g., a solid tumor. In some embodiments, the subject is suffering from a cancer. In some embodiments, the pluripotent stem cell, the differentiated cell, the progeny or daughter cell, or the population of cells is allogeneic to the subject. In some embodiments, the subject is a human.

In another aspect, the disclosure features a method of manufacturing a cell. In some embodiments, the method comprises: (a) knocking-out a gene of the cell, wherein the gene encodes Beta-2-Microglobulin (B2M); and (b) knocking-in to the genome of the cell an exogenous nucleic acid comprising a nucleotide sequence encoding an HLA-E polypeptide, wherein the exogenous nucleic acid is knocked-in in frame and downstream (3′) of an essential gene.

In some embodiments, knocking-out comprises contacting the cell with an RNP complex comprising: (i) an RNA-guided nuclease, and (ii) a guide RNA comprising a targeting domain sequence comprising a nucleotide sequence selected from the group consisting of SEQ ID NO: 365-576. In some embodiments, the guide RNA comprises a targeting domain sequence comprising the nucleotide sequence of SEQ ID NO: 412.

In some embodiments, knocking-in comprises contacting the cell with: (i) a nuclease that causes a break within an endogenous coding sequence of the essential gene, and (ii) a donor template that comprises a knock-in cassette comprising the exogenous nucleic acid in frame with and downstream (3′) of an exogenous coding sequence or partial coding sequence of the essential gene, wherein the knock-in cassette is integrated into the genome of the cell by homology-directed repair (HDR) of the break.

In some embodiments, the nuclease is an RNA-guided nuclease. In some embodiments, the RNA-guided nuclease comprises Cas9, Cas12a, Cas12b, Cas12c, Cas12e, CasX, or CasΦ (Cas12j), or a variant thereof, e.g., a variant capable of editing about 60% to 100% of cells in a population of cells. In some embodiments, the RNA-guided nuclease is a Cas12a variant. In some embodiments, the Cas12a variant comprises one or more amino acid substitutions selected from M537R. F870L, and H800A. In some embodiments, the Cas12a variant comprises amino acid substitutions M537R, F870L, and H800A. In some embodiments, the Cas12a variant comprises an amino acid sequence having 90%, 95%, or 100% identity to SEQ ID NO: 1148. In some embodiments, knocking-in further comprises contacting the cell with a guide RNA for the RNA-guided nuclease. In some embodiments, the guide RNA comprises a targeting domain sequence comprising or consisting of a nucleotide sequence that is identical to, or differs by no more than 1, 2, or 3 nucleotides from, SEQ ID NO: 1178.

In some embodiments, the cell is a pluripotent stem cell, e.g., an induced pluripotent stem cell (iPSC). In some embodiments, the cell is a differentiated cell. In some embodiments, the cell is an induced NK (iNK) cell.

In some embodiments, the essential gene is a housekeeping gene, e.g., a gene listed in Table 13. In some embodiments, the essential gene encodes glyceraldehyde 3-phosphate dehydrogenase (GAPDH).

In some embodiments, the method further comprises knocking-out one or more genes of the cell, wherein the one or more genes encode an agonist of the TGF beta signaling pathway, Cytokine Inducible SH2 Containing Protein (CISH), class II, major histocompatibility complex, transactivator (CIITA), and/or adenosine A2a receptor (ADORA2A), or any combination of two or more thereof.

In another aspect, the disclosure features a method of reducing a level of killing of a population of cells by NK cells, the method comprising: (a) knocking-out a gene of cells of the population, wherein the gene encodes Beta-2-Microglobulin (B2M); and (b) knocking-in to the genome of the cells of the population an exogenous nucleic acid comprising a nucleotide sequence encoding an HLA-E polypeptide, wherein the exogenous nucleic acid is knocked-in in frame and downstream (3′) of an essential gene; thereby reducing the level of killing of the population of cells when contacted with NK cells (e.g., by at least about 10%, 20%, 40%, 60%, 80%, or 100%) relative to a reference level of killing of a reference population of cells when contacted with NK cells (as determined using, e.g., a method described herein). In some embodiments, the NK cells are human donor NK cells and/or peripheral blood NK cells.

In some embodiments, the reference population of cells does not comprise iNK cells comprising a genome comprising the exogenous nucleic acid. In some embodiments, the reference population of cells does not comprise iNK cells comprising the genomic edit that results in loss of function of B2M. In some embodiments, the reference population of cells comprises iNK cells that are the same as the population of genomically edited iNK cells, but whose genomes do not comprise the exogenous nucleic acid (e.g., encoding the HLA-E polypeptide) and whose genomes do not comprise the genomic edit that results in loss of function of B2M.

In some embodiments, knocking-out comprises contacting the population of cells with an RNP complex comprising: (i) an RNA-guided nuclease, and (ii) a guide RNA comprising a targeting domain sequence comprising a nucleotide sequence selected from the group consisting of SEQ ID NO: 365-576. In some embodiments, the guide RNA comprises a targeting domain sequence comprising the nucleotide sequence of SEQ ID NO: 412.

In some embodiments, knocking-in comprises contacting the population of cells with: (i) a nuclease that causes a break within an endogenous coding sequence of the essential gene, and (ii) a donor template that comprises a knock-in cassette comprising the exogenous nucleic acid in frame with and downstream (3′) of an exogenous coding sequence or partial coding sequence of the essential gene, wherein the knock-in cassette is integrated into the genome of cells of the population by homology-directed repair (HDR) of the break.

In some embodiments, the nuclease is an RNA-guided nuclease. In some embodiments, the RNA-guided nuclease comprises Cas9, Cas12a, Cas12b, Cas12c, Cas12c, CasX, or CasΦ (Cas12j), or a variant thereof, e.g., a variant capable of editing about 60% to 100% of cells in a population of cells. In some embodiments, the RNA-guided nuclease is a Cas12a variant. In some embodiments, the Cas 12a variant comprises one or more amino acid substitutions selected from M537R, F870L, and H800A. In some embodiments, the Cas12a variant comprises amino acid substitutions M537R, F870L, and H800A. In some embodiments, the Cas12a variant comprises an amino acid sequence having 90%, 95%, or 100% identity to SEQ ID NO: 1148. In some embodiments, knocking-in further comprises contacting the population of cells with a guide RNA for the RNA-guided nuclease. In some embodiments, the guide RNA comprises a targeting domain sequence comprising or consisting of a nucleotide sequence that is identical to, or differs by no more than 1, 2, or 3 nucleotides from, SEQ ID NO: 1178.

In some embodiments, the population of cells comprises pluripotent stem cells, e.g., induced pluripotent stem cells (iPSCs). In some embodiments, the population of cells comprises differentiated cells. In some embodiments, the population of cells comprises induced NK (INK) cells.

In some embodiments, the essential gene is a housekeeping gene, e.g., a gene listed in Table 13. In some embodiments, the essential gene encodes glyceraldehyde 3-phosphate dehydrogenase (GAPDH).

In some embodiments, the method further comprises knocking-out one or more genes of cells of the population, wherein the one or more genes encode an agonist of the TGF beta signaling pathway, Cytokine Inducible SH2 Containing Protein (CISH), class II, major histocompatibility complex, transactivator (CIITA), and/or adenosine A2a receptor (ADORA2A), or any combination of two or more thereof.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 shows microscopy of cell morphology and flow cytometry of pluripotency markers of human induced pluripotent stem cells (hiPSCs) grown in various media in the absence or presence of Activin A (1 ng/ml or 4 ng/ml ActA).

FIG. 2 shows morphology of TGFβRII knockout hiPSCs (clone 7) or CISH/TGFβRII DKO hiPSCs (clone 7) cultured in media with or without Activin A (1 ng/mL, 2 ng/mL, 4 ng/mL, or 10 ng/ml).

FIG. 3 shows morphology of TGFβRII knockout hiPSCs (clone 9) cultured in media with or without Activin A (1 ng/mL, 2 ng/mL, 4 ng/mL, or 10 ng/ml).

FIG. 4A shows the bulk editing rates at the CISH and TGFβRII loci for single knockout and double knockout hiPSCs.

FIG. 4B shows expression of Oct4 and SSEA4 in TGFβRII knockout hiPSCs, CISH knockout hiPSCs, and double knockout hiPSCs cultured in Activin A.

FIG. 5 shows expression of Nanog and Tra-1-60 in TGFβRII knockout hiPSCs, CISH knockout hiPSCs, and double knockout hiPSCs cultured in Activin A.

FIG. 6 is a schematic of the procedure related to the STEMdiff™ Trilineage Differentiation Kit (STEMCELL Technologies Inc.).

FIG. 7A shows expression of differentiation markers of TGFβRII knockout hiPSCs, CISH knockout hiPSCs, and double knockout hiPSCs cultured in Activin A.

FIG. 7B shows karyotypes of TGFβRII/CISH double knockout hiPSCs cultured in Activin A.

FIG. 7C shows an expanded Activin A concentration curve performed on an unedited parental PSC line, an edited TGFβRII KO clone (C7), and an additional representative (unedited) cell line designated RUCDR. The minimum concentration of Activin A required to maintain each line varied slightly with the TGFβRII KO clone requiring a higher baseline amount of Activin A as compared to the parental control (0.5 ng/ml vs 0.1 ng/ml).

FIG. 7D shows the stemness marker expression in an unedited parental PSC line, an edited TGFβRII KO clone (C7), and an unedited RUCDR cell line, when cultured with the base medias alone (no supplemental Activin A). The TGFβRII KO iPSCs did not maintain stemness marker expression while the two unedited lines were able to maintain stemness marker expression in E8.

FIG. 8A is a schematic representation of an exemplary method for creating edited iPSC clones, followed by the differentiation to and characterization of enhanced CD56+ iNK cells.

FIG. 8B is a schematic of an iNK cell differentiation process utilizing STEMDiff APEL2 during the second stage of the differentiation process.

FIG. 8C is a schematic of an iNK cell differentiation process utilizing NK-MACS with 15% serum during the second stage of the differentiation process.

FIG. 8D shows the fold-expansion of unedited PCS-derived iNK cells and the percentage of iNK cells expressing CD45 and CD56 at day 39 of differentiation when differentiated using NK-MACS or Apel2 methods as depicted in FIG. 8C and FIG. 8B respectively.

FIG. 8E shows in the upper panel a heat map of the surface expression phenotypes (measured as a percentage of the population) of differentiated iNK cells derived from unedited PCS iPSCs when differentiated using NK-MACS or APEL2 methods as depicted in FIG. 8C and FIG. 8B respectively. The bottom panel displays representative histogram plots to illustrate the differences in the iNKs generated by the two methods.

FIG. 8F shows a heat map of the surface expression phenotypes (measured as a percentage of the population) of differentiated edited iNKs (TGFβRII knockout, CISH knockout, and double knockout (DKO)) and unedited parental iPSCs (WT) when differentiated using NK-MACS or APEL2 methods as depicted in FIG. 8C and FIG. 8B respectively.

FIG. 8G shows unedited iNK cell effector function when differentiated using NK-MACS or APEL2 methods as depicted in FIG. 8C and FIG. 8B respectively.

FIG. 9 shows differentiation phenotypes of edited clones (TGFβRII knockout, CISH knockout, and double knockout) as compared to parental wild type clones.

FIG. 10 shows surface expression phenotype of edited iNKs (TGFβRII knockout, CISH knockout, and double knockout) as compared to parental clone iNKs and wild type cells.

FIG. 11A shows surface expression phenotype of edited iNKs (TGFβRII knockout, CISH knockout, and double knockout) as compared to parental clone iNKs (“WT”) and peripheral blood-derived natural killer cells.

FIG. 11B is a flow cytometry histogram plot that shows the surface expression phenotype of edited iNK cells (TGFβRII/CISH double knockout) as compared to parental clone iNK cells (“unedited iNK cells”).

FIG. 11C shows surface expression phenotypes (measured as a percentage of the population) of edited iNK cells (TGFβRII/CISH double knockout) as compared to parental clone iNK cells (“unedited iNK cells”) at day 25, day 32, and day 39 post-hiPSC differentiation (average values from at least 5 separate differentiations).

FIG. 11D shows pSTAT3 expression phenotypes (measured as a percentage of the population) of edited CD56+ iNK cells (“CISH KO iNKs”) as compared to parental clone CD56+ iNK cells (“unedited iNKs”) at 10 minutes and 120 minutes following IL-15 induced activation. Briefly, the day 39 or day 40 iNKs are plated the day before in a cytokine starved condition. The next day the cells are stimulated with 10 ng/ml of IL15 for the length of time indicated. The cells are fixed immediately at the end of the time point, stained for CD56 followed by an intracellular stain. The cells were processed on a NovoCyte Quanteon and the data was analyzed in FlowJo. Data shown is a representative experiment of >3 experiments performed.

FIG. 11E shows pSMAD2/3 expression phenotypes (measured as a percentage of the population) of edited CD56+ iNK cells (TGFβRII/CISH double knockout, “DKO iNKs”) as compared to parental clone CD56+ iNK cells (“unedited iNK cells”) at 10 minutes and 120 minutes following IL-15 and TGF-β induced activation Briefly, the day 39 or day 40 iNKs were plated the day before in a cytokine starved condition. The next day the cells were stimulated with 10 ng/ml of IL-15 and 50 ng/ml of TGF-β for the length of time indicated. The cells were fixed immediately at the end of the time point, stained for CD56 followed by an intracellular stain. The cells were processed on a NovoCyte Quanteon and the data was analyzed in FlowJo. Data shown is a representative experiment of >3 experiments performed.

FIG. 11F shows IFN-γ expression phenotypes (measured as a percentage of the population) of edited CD56+ iNK cells (TGFβRII/CISH double knockout, “DKO IFNg”) as compared to parental clone CD56+ iNK cells (unedited iNKs, “WT IFNg”) with or without phorbol myristate acetate (PMA) and ionomycin (IMN) stimulation. The data is representative. It is generated from a single differentiation and each condition in the assay is run with 2 technical replicates. **p<0.05 vs unedited iNK cells (paired t test).

FIG. 11G shows TNF-α expression phenotypes (measured as a percentage of the population) of edited CD56+ iNK cells (TGFβRII/CISH double knockout, “DKO TNF a”) as compared to parental clone CD56+ iNK cells (unedited iNK cells, “WT TNFa”) with or without Phorbol myristate acetate (PMA) and Ionomycin (IMN) stimulation. The data is representative. It is generated from a single differentiation and each condition in the assay is run with 2 technical replicates. **p<0.05 vs unedited iNK cells (paired t test).

FIG. 12A is a schematic representation of an exemplary solid tumor cell killing assay, depicting the use of edited iNK cells (TGFβRII/CISH double knockout) to kill SK-OV-3 ovarian cells in the presence or absence of IL-15 and TGF-β.

FIG. 12B shows the results of a solid tumor killing assay as described in FIG. 12A. iNK cells function to reduce tumor cell spheroid size. Certain edited iNK cells (CISH single knockout, “CISH_2, 4, 5, and 8”) were not significantly different from the parental clone iNK cells (“WT_2”), while certain edited iNK cells (TGFβRII single knockout, “TGFβRII_7”, and TGFβRII/CISH double knockout “DKO”) functioned significantly better at effector-target (E:T) ratios of 1 or greater when measured in the presence of TGF-β as compared to parental clone iNK cells (“WT_2”). ****p<0.0001 vs unedited iNK cells (two-way ANOVA, Sidak's multiple comparisons test).

FIG. 12C shows edited iNK cell effector function as compared to unedited iNK cells.

FIG. 13 shows the results of an in-vitro serial killing assay, where iNK cells are serially challenged with hematological cancer cells (e.g., Nalm6 cells) in the presence of 10 ng/ml of IL-15 and 10 ng/ml of TGF-β; the X axis represents time, with tumor cells being added every 48 hours, while the Y axis represents killing efficacy as measured by normalized total red object area (e.g., presence of tumor cells). The data shows that edited iNK cells (TGFβRII/CISH double knockout) continue to kill hematological cancer cells while unedited iNK cells lose this function at equivalent time points.

FIG. 14 shows surface expression phenotypes (measured as a percentage of the population) of certain edited iNK clonal cells (CISH single knockout “CISH_C2, C4, C5, and C8”, TGFβRII single knockout “TGFβRII-C7”, and TGFβRII/CISH double knockout “DKO-C1”) as compared to parental clone iNK cells (“WT”) at day 25, day 32, and day 39 post-hiPSC differentiation when cultured in the presence of 1 ng/ml or 10 ng/ml IL-15.

FIG. 15A is a schematic of an in-vivo tumor killing assay. Mice were intraperitoneally inoculated with 1×10⁶ SKOV3-luc cells, mice are randomized, and 4 days later, 20×10⁶ iNK cells were introduced intraperitoneally. Mice were followed for up to 60 days post-tumor implantation. The X axis represents time since implantation, while the Y axis represents killing efficacy as measured by total bioluminescence (p/s).

FIG. 15B shows the results of an in-vivo tumor killing assay as described in FIG. 15A. An individual mouse is represented by each horizontal line. The data show that both unedited iNK cells (“unedited iNK”) and DKO edited iNK cells (TGFβRII/CISH double knockout) prevent tumor growth better than vehicle, while edited iNK cells kill tumor cells significantly better than vehicle in-vivo. Each experimental group had 9 animals each. ***p<0.001. ****p<0.0001 by a 2-way ANOVA analysis.

FIG. 15C shows the averaged results with standard error of the mean of the in-vivo tumor killing assay described in FIG. 15B. Populations of mice are represented by each horizontal line. The data show that DKO edited iNK cells (TGFβRII/CISH double knockout) prevent tumor growth and kill tumor cells significantly better than vehicle or unedited iNK cells in-vivo. ***p<0.001. ****p<0.0001 by a 2-way ANOVA analysis.

FIG. 16A shows surface expression phenotypes (measured as a percentage of the population) of bulk edited iNK cells (left panel-ADORA2A single knockout) or certain edited iNK clonal cells (right panel-ADORA2A single knockout) as compared to parental clone iNK cells (“PCS_WT”) at day 25, day 32, and day 39 or at day 28, day 36, and day 39 post-hiPSC differentiation. Representative data from multiple differentiations.

FIG. 16B shows cyclic AMP (CAMP) concentration phenotypes following 5′-(N-Ethylcarboxamido)adenosine (“NECA”, adenosine agonist) activation for edited iNK clonal cells (ADORA2A single knockout) as compared to parental clone iNK cells (“unedited iNKs”). The Y axis represents average cAMP concentration in nM (a proxy for ADORA2A activation), while the X axis represents NECA concentration in nM.

FIG. 16C shows the results of an in-vitro serial killing assay, where iNK cells are serially challenged with hematological cancer cells (e.g., Nalm6 cells) in the presence of 100 μM NECA, and 10 ng/ml of IL-15; the X axis represents time, with tumor cells being added every 48 hours, while the Y axis represents killing efficacy as measured by total red object area (e.g., presence of tumor cells). The data shows that edited iNK cells (“ADORA2A KO INK”) kill hematological cancer cells more effectively than unedited iNK cells (“Ctrl iNK”) under conditions that mimic adenosine suppression.

FIG. 17A shows surface expression phenotypes (measured as a percentage of the population) of certain edited iNK clonal cells (TGFβRII/CISH/ADORA2A triple knockout, “CRA_6” and “CR+A_8”) as compared to parental clone iNK cells (“WT_2”) at day 25, day 32, and day 39 post-hiPSC differentiation. Data is representative of multiple differentiations.

FIG. 17B shows cyclic AMP (cAMP) concentration phenotypes following NECA (adenosine agonist) activation for edited iNK clonal cells (TGFβRII/CISH/ADORA2A triple knockout, “TKO iNKs”) as compared to parental clone iNK cells (“unedited iNKs”). The Y axis represents average cAMP concentration in nM (a proxy for ADORA2A activation), while the X axis represents NECA concentration in nM.

FIG. 17C shows the results of a solid tumor killing assay as described in FIG. 12A without IL-15. iNK cells function to reduce tumor cell spheroid size. The Y axis measures total integrated red object (e.g., presence of tumor cells), while the X axis represents the effector to target (E:T) cell ratio. The edited iNK cells (ADORA2A single knockout “ADORA2A”, TGFβRII/CISH double knockout “DKO”, or TGFβRII/CISH/ADORA2A triple knockout “TKO”) had lower EC50 rates when measured in the presence of TGF-β as compared to parental clone iNK cells (“Control”) (average values from at least 3 separate differentiations).

FIG. 18 shows the results of guide RNA selection assays for the loci TGFβRII, CISH, ADORA2A, TIGIT, and NKG2A utilizing in-vitro editing in iPSCs.

FIG. 19A shows an exemplary integration strategy that targets an essential gene according to certain embodiments of the present disclosure. In particular embodiments, introducing a double strand break using CRISPR gene editing (e.g., by Cas12a or Cas9) within a terminal exon (e.g., within about 500 bp upstream (5′) of the stop codon of the essential gene) and administering a donor plasmid with homology arms designed to mediate homology directed repair (HDR) at the cleavage site, results in a population of viable cells carrying a cargo of interest integrated at the essential gene locus. Those cells that were edited by the CRISPR nuclease, but failed to undergo integration of the cargo at the essential gene locus, do not survive.

FIG. 19B shows an exemplary integration strategy that targets the GAPDH gene according to certain embodiments of the present disclosure. Although FIG. 19B shows a strategy wherein the GAPDH gene is modified in an induced pluripotent stem cell (iPSC), this strategy can be applied to a variety of cell types, including primary cells, stem cells, and cells differentiated from iPSCs.

FIG. 19C shows an exemplary integration strategy that targets the GAPDH gene according to certain embodiments of the present disclosure. The diagram shows that the only cells that should survive over time are those cells that underwent targeted integration of a cassette that restores the GAPDH locus and includes a cargo of interest, as well as unedited cells. The population of unedited cells following CRISPR editing should be small if the nuclease and guide RNA are highly effective at cleaving the essential gene target site and introduce indels that significantly reduce the function of the essential gene product.

FIG. 19D shows an exemplary integration strategy that targets an essential gene according to certain embodiments of the present disclosure. In particular embodiments, introducing a double strand break using CRISPR gene editing (e.g., by Cas12a or Cas9) to target a 5′ exon (e.g., within about 500 bp downstream (3′) of a start codon of the essential gene) and administering a donor plasmid with homology arms designed to mediate homology directed repair (HDR) at the cleavage site, results in a population of viable cells carrying a cargo of interest integrated at the essential gene locus. Those cells that were edited by the CRISPR nuclease, but failed to undergo integration of the cargo at the essential gene locus, do not survive.

FIG. 19E shows the efficiency of integration of a knock-in cassette, comprising a GFP protein encoding “cargo” sequence, into the GAPDH locus of iPSCs, measured 7 days following transfection. Depicts exemplary flow cytometry data showing insertion rates for cargo transfection alone (PLA1593 or PLA1651) compared to cargo and guide RNA transfections (RSQ22337+PLA1593 or RSQ24570+PLA1651), additionally, insertion rates with an exemplary exonic coding region targeting guide RNA with appropriate cargo (RSQ22337+PLA 1593) are compared to insertion rates with an intronic targeting guide RNA with appropriate cargo (RSQ24570+PLA1651).

FIG. 20A depicts a schematic representation of a bicistronic knock-in cassette (e.g., comprising two cistrons separated by a linker) for insertion into the GAPDH locus. The leading GAPDH Exon 9 coding region and exogenous sequences encoding proteins of interest are separated by linker sequences, and the second GAPDH allele can comprise a target knock-in cassette insertion, indels, or is wild type (WT).

FIG. 20B depicts a schematic representation of bi-allelic knock-in cassettes for insertion into the GAPDH locus. Exogenous “cargo” sequences encoding proteins of interest are located on different knock-in cassettes. For each construct, the leading GAPDH Exon 9 coding region is separated from an exogenous sequence encoding a protein of interest by a linker sequence.

FIG. 20C depicts a schematic representation of a bicistronic knock-in cassette for insertion into the GAPDH locus, with the leading GAPDH Exon 9 coding region and exogenous sequences encoding GFP and mCherry separated by linker sequences P2A, T2A, and/or IRES.

FIG. 20D depicts expression quantification (Y axis) of exemplary “cargo” molecules GFP and mCherry from various bicistronic molecules comprising the described linker pairs (X axis). mCherry as a sole “cargo” protein was utilized as a relative control. iPSCs were quantified by flow-cytometry nine days following nucleofection of RNPs comprising RSQ22337 (SEQ ID NO: 1178) targeting GAPDH and Cas12a (SEQ ID NO: 1148) and a bicistronic knock-in cassette comprising “cargo” sequence encoding GFP and mCherry molecules inserted at the GAPDH locus. iPSCs comprising exemplary “cargo” molecules PLA1582 (data not shown) with linkers P2A and T2A, PLA1583 (data not shown) with linkers T2A and P2A, and PLA1584 (data not shown) with linkers T2A and IRES are shown. Results show that at least two different cargos can be inserted in a bicistronic manner and expression is detectable irrespective of linker type used.

FIG. 20E are histograms depicting exemplary flow cytometry analysis data for bi-allelic GFP and mCherry knock-in at the GAPDH gene. Cells were nucleofected with 0.5 μM RNPs comprising Cas12a (SEQ ID NO: 1148) and RSQ22337 (SEQ ID NO: 1178), and 2.5 μg (5 trials) or 5 μg (1 trial) GFP and mCherry donor templates.

FIG. 21A depicts exemplary flow cytometry data for GFP expression in iPSCs seven days after being transfected with a gRNA and an appropriate donor template comprising a knock-in cassette with a “cargo” sequence encoding GFP that was recombined into various loci.

FIG. 21B depicts the percentage of cells having editing events as measured by Inference of CRISPR Edits (ICE) assays 48 hours after being transfected with the noted gRNA.

FIG. 22 depicts the percentage of WT iNK cells or B2M KO iNK cells undergoing specific lysis (y-axis, top panel) or the percentage of live iNK cells (y axis, bottom panel) following in-vitro overnight (16 hour) co-culture exposure to Human Derived Natural Killer (HDNK) cells at various E:T ratios (x axis, both panels); representative data from two HDNK donors and two independent experiments. The data show B2M KO iNKs are more susceptible to HDNK cytotoxicity.

FIG. 23 depicts the percentage of HDNKs expressing degranulation marker CD107a (y-axis) following overnight 1:1 (E:T) co-culture with the noted cell type (x-axis). The myelogenous leukemia cell line, K562, potently activates HDNKs. Additionally, at day 39 of differentiation to iNKs, WT iPSC derived iNKs activate significantly fewer HDNKs when compared to B2M KO iNKs; N=5 (3 donors) from two independent experiments, **p<0.01, by ANOVA. These data indicate that, without additional intervention, B2M KO iNK may quickly be depleted by recipient HDNKs.

FIG. 24A depicts K562 cell expression of CD47 isoform 2 (WT or S64A; represented by SEQ ID NO: 1183) driven by an EF1α promoter and introduced via lentiviral mediated transduction. K562 cells were transduced with an MOI of 10 using spinfection, stained 48 hours post-transduction, and expression was measured using flow-cytometry (Geometric Mean Fluorescence Intensity (gMFI)).

FIG. 24B depicts K562 cell expression of an HLA-E trimer (represented by SEQ ID NO: 1181) driven by an EF1α promoter and introduced via lentiviral mediated transduction. K562 cells were transduced with an MOI of 10 using spinfection, stained 48 hours post-transduction, and expression was measured using flow-cytometry (Geometric Mean Fluorescence Intensity (gMFI)).

FIG. 24C depicts K562 cell expression of an HLA-G trimer (represented by SEQ ID NO: 1179) driven by an EF1α promoter and introduced via lentiviral mediated transduction. K562 cells were transduced with an MOI of 10 using spinfection, stained 48 hours post-transduction, and expression was measured using flow-cytometry (Geometric Mean Fluorescence Intensity (gMFI)).

FIG. 25A depicts the percentage of HDNKs expressing degranulation marker CD107a (y-axis) following overnight 1:1 (E:T) co-culture with vehicle (NK alone), K562 cells, or K562 cells expressing CD47 (transduced as described in FIG. 24A).

FIG. 25B depicts the percentage of HDNKs expressing degranulation marker CD107a (y-axis) following overnight 1:1 (E:T) co-culture with vehicle (NK alone), K562 cells, or K562 cells expressing HLA-G (transduced as described in FIG. 24B).

FIG. 25C depicts the percentage of HDNKs expressing degranulation marker CD107a (y-axis) following overnight 1:1 (E:T) co-culture with vehicle (NK alone), K562 cells, or K562 cells expressing HLA-E (transduced as described in FIG. 24C); representative data shown, 3 donor HDNK cells. ***p<0.001, by ANOVA. These data indicate that expression of HLA-E can effectively shield K562 cells from activating HDNKs, reducing the percentage of HDNKs expressing CD107a.

FIG. 25D depicts the percentage of HDNK cells expressing degranulation marker CD107a (y-axis) in response to overnight 1:1 (E:T) co-culture with vehicle (NK alone), WT K562 cells, or HLA-E expressing K562 cells as a function of HDNK cell NKG2A and/or NKG2C expression status (x-axis). HDNK cell populations labeled NKG2A+ are NKG2C−, HDNK cell populations labeled NKG2C+ are NKG2A−, and HDNK cell populations labeled NKG2A+NKG2C+ represent double positive populations for these markers. These data indicate that transgenic HLA-E expression (SEQ ID NO: 1181) in K562 cells can effectively inhibit NKG2A+ mediated HDNK degranulation. For each HDNK cell population listed on the x-axis, the three bars above representing % CD107a+correspond, in order from left to right, to “NK Alone”, “WT”, and “HLA-E”.

FIG. 25E depicts the percentage of HDNK cells expressing degranulation marker CD107a (y-axis) in response to overnight 1:1 (E:T) co-culture with WT K562 cells or HLA-E expressing K562 cells. HDNK cell populations were either NKG2A+ or NKG2A− as indicated. These data indicate that transgenic HLA-E expression (SEQ ID NO: 1181) in K562 cells can effectively inhibit NKG2A+ mediated HDNK degranulation. N=3 technical replicates from N=3 unique samples; error bars represent standard deviation, **p<0.01.

FIG. 26A depicts the percentage of dead (y-axis) WT K562 cells or CD47 expressing K562 cells following overnight incubation with HDNKs at noted E:T ratios (x-axis); representative data shown, 3 donor HDNK cells.

FIG. 26B depicts the percentage of dead (y-axis) WT K562 cells or HLA-G expressing K562 cells following overnight incubation with HDNKs at noted E:T ratios (x-axis); representative data shown, 3 donor HDNK cells.

FIG. 26C depicts the percentage of dead (y-axis) WT K562 cells or HLA-E expressing K562 cells following overnight incubation with HDNKs at noted E:T ratios (x-axis); representative data shown, 3 donor HDNK cells. These data indicate that transgenic HLA-E protects K562 cells from HDNK cytotoxicity.

FIG. 27A depicts CD56 or MHC class 1 (HLA-1) surface expression in WT iPSCs at day 47 of differentiation to iNK cells; the percentage of cells expressing CD56 was ˜92%, and the percentage of cells expressing HLA-1 was ˜85%; representative data from 2 independent experiments, measured using flow cytometry.

FIG. 27B depicts CD56 or MHC class 1 (HLA-1) surface expression in B2M KO iPSCs at day 47 of differentiation to iNK cells; the percentage of cells expressing CD56 was ˜95%, and the percentage of cells expressing HLA-1 was ˜3%; representative data from 2 independent experiments, measured using flow cytometry.

FIG. 28A depicts the percentages of CD4+ T cells that have proliferated (y-axis) following Mixed Lymphocyte Reaction (MLR) experiments comprising PBMC responders Aph10, Aph11, Aph13, or CEL346 (x-axis) that have undergone overnight co-culture at a 2:1 (E:T) ratio (100K PBMC to 50K iNK) with the noted stimulators (vehicle (cytokine only), B2M KO iNKs, WT iNKs, or activation beads). Collated results from two independent experiments (day 44 and day 48 of differentiation from iPSC to iNK), cells were cultured in X-vivo15 Media with 5% AB serum, 100iU/IL-2, and 20 ng/IL-15. For each PBMC responder on the x-axis, the four bars above representing % Proliferated of CD4+ T cells correspond, in order from left to right, to “+Vehicle (cytokine only)”, “+ B2M KO iPSC iNKs”, “+WT iPSC iNK”, and “+Activation Beads”.

FIG. 28B depicts the percentages of CD8+ T cells that have proliferated (y-axis) following MLR experiments comprising PBMC responders Aph10, Aph11, Aph13, or CEL346 (x-axis) that have undergone overnight co-culture at a 2:1 (E:T) ratio (100K PBMC to 50K iNK) with the noted stimulators (vehicle (cytokine only), B2M KO iNKs, WT iNKs, or activation beads). Collated results from two independent experiments (day 44 and day 48 of differentiation from iPSC to iNK), cells were cultured in X-vivo15 Media with 5% AB serum, 100iU/IL-2, and 20 ng/IL-15. The average percentage of CD8+ T cells proliferating in response to B2M KO iNKs was lower than for WT iNKs. For each PBMC responder on the x-axis, the four bars above representing % Proliferated of CD4+ T cells correspond, in order from left to right, to “+Vehicle (cytokine only)”, “+ B2M KO iPSC iNKs”, “+WT iPSC iNK”, and “+Activation Beads”.

FIG. 29A depicts the percentages of CD4+ T cells that have proliferated (y-axis) following MLR experiments comprising PBMC responders Aph10, Aph11, Aph13, or CEL346 (x-axis) that have undergone overnight co-culture at a 2:1 (E:T) ratio (100K PBMC to 50K iNK) with the noted stimulators (vehicle (cytokine only), B2M KO iNKs Clone 5 (C5), B2M KO iNKs Clone 11 (C11), B2M/CIITA DKO iNKs Clone 10 (C10), WT iNKs, or activation beads). Collated results from two independent experiments (day 44 and day 48 of differentiation from iPSC to iNK), cells were cultured in X-vivo15 Media with 5% AB serum, 100iU/IL-2, and 20 ng/IL-15. The data show enhanced CD4+ T cell alloresponse to MHC-II++ iNKs. For each PBMC responder on the x-axis, the four bars above representing % Proliferated of CD4+ T cells correspond, in order from left to right, to “+Vehicle (cytokine only)”, “+ B2M KO iPSC INK, C5”, “+ B2M KO iPSC INK, C11”, “+ B2M/CIITA DKO iPSC INK, C10”, “+WT iPSC iNK”, and “+Activation Beads”.

FIG. 29B depicts the percentages of CD8+ T cells that have proliferated (y-axis) following MLR experiments comprising PBMC responders Aph10, Aph11, Aph13, or CEL346 (x-axis) that have undergone overnight co-culture at a 2:1 (E:T) ratio (100K PBMC to 50K iNK) with the noted stimulators (vehicle (cytokine only), B2M KO iNKs Clone 5 (C5), B2M KO iNKs Clone 11 (C11), B2M/CIITA DKO iNKs Clone 10 (C10), WT iNKs, or activation beads). Collated results from two independent experiments (day 44 and day 48 of differentiation from iPSC to iNK), cells were cultured in X-vivo15 Media with 5% AB serum, 100iU/IL-2, and 20 ng/IL-15. The average percentage of CD8+ T cells proliferating in response to B2M KO iNKs was lower than for WT iNKs. For each PBMC responder on the x-axis, the four bars above representing % Proliferated of CD4+ T cells correspond, in order from left to right, to “+Vehicle (cytokine only)”. “+ B2M KO iPSC INK, C5”, “+ B2M KO iPSC INK, C11”, “+ B2M/CIITA DKO iPSC INK, C10”, “+WT iPSC iNK”, and “+Activation Beads”.

FIG. 29C is a representative flow cytometry plot depicting MHC-1 expression (y-axis) and MHC-II expression (x-axis) in B2M KO iPSC derived iNK cells from Clone 5 (C5). Approximately 96% of cells were negative for both MHC-1 and MHC-II.

FIG. 29D is a representative flow cytometry plot depicting MHC-1 expression (y-axis) and MHC-II expression (x-axis) in B2M KO iPSC derived iNK cells from Clone 11 (C11). Approximately 82% of cells were negative for both MHC-1 and MHC-II, while approximately 17% of cells were positive for MHC-II only.

FIG. 29E is a representative flow cytometry plot depicting MHC-1 expression (y-axis) and MHC-II expression (x-axis) in B2M/CIITA DKO iPSC derived iNK cells from Clone 10 (C10). Approximately 97% of cells were negative for both MHC-1 and MHC-II.

FIG. 30A depicts percentages of cell populations positive (y-axis) for transgenic markers determined by flow cytometry for various B2M KO iPSC clonal cell lines (x-axis) with transgenic CD47 expression (Clones 10 and 12), transgenic HLA-E expression (Clones 2 and 18), or transgenic HLA-G expression (Clones 1 and 16) pre-differentiation (left panel) or at day 31 post-differentiation to iNKs (right panel). A high percentage of C18 derived iNKs expressed HLA-E.

FIG. 30B depicts RT-qPCR ddCT values (y-axis) for various B2M KO iPSC derived iNKs expressing transgenic CD47 expression (Clones 10 and 12), transgenic HLA-E expression (Clones 2 and 18), or transgenic HLA-G expression (Clone 1) at day 31 post-differentiation to iNKs (x-axis). The majority of C18 derived iNKs robustly expressed HLA-E mRNA relative to wild type iNKs.

FIG. 31A depicts the percentage of HDNKs expressing degranulation marker CD107a (y-axis) following overnight 1:1 (E:T) co-culture with WT iPSC derived iNKs (WT), B2M KO iPSC derived iNKs (B2M KO), or B2M KO iPSC derived iNKs expressing transgenic HLA-E (B2M KO+HLA-E). The data show HLA-E protects B2M KO iNKs from HDNK cytotoxicity. Representative data collated from 5 donors; error bars represent SEM; *P<0.05 by ANOVA.

FIG. 31B depicts the percentage of HDNK cells expressing degranulation marker CD107a (y-axis) in response to overnight 1:1 (E:T) co-culture with WT iPSC derived iNKs (WT), B2M KO iPSC derived iNKs (B2M KO), or B2M KO iPSC derived iNKs expressing transgenic HLA-E (B2M KO+HLA-E). HDNK cell populations labeled NKG2A+ are NKG2C−, HDNK cell populations labeled NKG2C+ are NKG2A−, and HDNK cell populations labeled NKG2A+NKG2C+represent double positive populations for these markers. These data indicate that transgenic HLA-E expression (SEQ ID NO: 1181) in B2M KO iNK cells can effectively inhibit NKG2A+mediated HDNK degranulation. Representative data collated from 5 donors; error bars represent SEM; *P<0.05. ***P<0.001 by ANOVA. For each HDNK cell population listed on the x-axis, the three bars above representing % CD107a+correspond, in order from left to right, to “WT”, “B2M KO”, and “B2M KO+HLA-E”.

FIG. 32A depicts HLA-E surface expression in T cells modified as described herein. Left panel depicts HLA-E surface expression in T cells transduced with AAV6 comprising a B2M-HLA-E cargo targeted for knock-in at GAPDH at 5E4 MOI and transformed with 1 μM of RNPs comprising Cas12a (SEQ ID NO: 1148) with RSQ22337 (SEQ ID NO: 1178), compared to mock transduced control cells (no AAV6 transduction). Right panel depicts expansion data for T cells comprising knock-in of the B2M-HLA-E cargo at GAPDH and expansion data for the mock transduced control T cells. Cells were stained with PE anti-human HLA-E antibody clone: 3D12 (1:100 dilution).

FIG. 32B depicts HLA-E or MHC1 surface expression in T cells modified as described herein. Left panel depicts HLA-E surface expression in T cells transduced with AAV6 comprising a B2M-HLA-E cargo targeted for knock-in at GAPDH at 5E4 MOI and transformed with a B2M targeting RNP and with 1 μM of RNPs comprising Cas12a (SEQ ID NO: 1148) with RSQ22337 (SEQ ID NO: 1178), compared to mock transduced control cells exposed to AAV6 only, without RNPs. Right panel depicts MHC1 surface expression in T cells transduced with AAV6 comprising a B2M-HLA-E cargo targeted for knock-in at GAPDH at 5E4 MOI and transformed with a B2M targeting RNP and with 1 μM of RNPs comprising Cas12a (SEQ ID NO: 1148) with RSQ22337 (SEQ ID NO: 1178), compared to mock transduced control cells exposed to AAV6 only without RNPs, or B2M KO control T cells.

FIG. 32C are representative flow cytometry plots depicting HLA-E expression (x-axis) and MHC-1 expression (y-axis) in T cells modified as described herein. Left panel depicts exemplary data from B2M KO control T cells. Right panel depicts exemplary data from T cells transduced with AAV6 comprising a B2M-HLA-E cargo targeted for knock-in at GAPDH at 5E4 MOI and transformed with a B2M-targeting RNP and with 1 μM of RNPs comprising Cas12a (SEQ ID NO: 1148) with RSQ22337 (SEQ ID NO: 1178).

FIG. 32D depicts exemplary data of the percentage of HDNK cells expressing degranulation marker CD107a (y-axis) in response to overnight culture alone (NK alone) or overnight 1:1 (E:T) co-culture with unedited T cells (Unedited), B2M KO control T cells (B2M KO), or B2M KO/B2M-HLA-E KI T cells (B2M KO HLA-E KI). These data indicate that transgenic HLA-E expression in B2M KO T cells can effectively inhibit HDNK degranulation. N=8, 4 independent donors in technical duplicate; horizontal bars represent median; ****p<0.0001 by one-way ANOVA test.

FIG. 33 are representative flow cytometry plots depicting MHC-1 expression (x-axis) and HLA-E expression (y-axis) or CD19 CAR expression (x-axis) and HLA-E expression (y-axis) in T cells modified as described herein. Each panel depicts exemplary data from T cells transformed with a donor template comprising CD19 CAR (SEQ ID NO: 1232) and B2M-HLA-E (NK Shield) (SEQ ID NO: 1230) separated by a P2A linker cargo targeted for knock-in at GAPDH, RNP comprising Cas12a (SEQ ID NO: 1148) with RSQ22337 (SEQ ID NO: 1178), and a B2M-targeting RNP.

FIG. 34A depicts multiplexed knock-out and knock-in efficiency in T cells as measured by a combination of next-generation sequencing (NGS) and flow cytometry (for phenotypic confirmation). TRAC (TCR) and/or B2M (MHC-I) were knocked out using targeted RNPs. CD19 CAR or GFP were knocked in by transformation with a corresponding donor template targeted for knock-in at GAPDH and a RNP comprising Cas12 (SEQ ID NO: 1148) with RSQ22337 (SEQ ID NO: 1178). The X axis denotes the edit (e.g., knock-out and/or knock-in), while the Y axis represents the percentage of cells containing the noted edit as determined by NGS and/or flow cytometry. Horizontal bars represent median, ns=not significant, ****p<0.0001.

FIG. 34B depicts the results of in vitro tumor cell killing assay, where T cells comprising CD19 CAR or GFP knock-in at the GAPDH gene (SLEEK KI) in combination with knock-out of TRAC, B2M, and CIITA (Triple KO) were challenged with hematological cancer cells (Nalm6 cells). Unedited T cells or T cells comprising CD19 CAR knock-in at the GAPDH alone were also tested. Significantly greater cytotoxicity was observed with T cells comprising CD19 CAR KI than T cells comprising GFP KI or unedited T cells as assessed by BATDA release following 24 hours of co-culture at an E:T of 1. Average spontaneous BATDA release by Nalm6 cells (dashed horizontal line) and average BATDA released upon treatment with lysis buffer (solid horizontal line) provided for comparison. Each circle represents data from 4 technical replicates from 1 biological sample. The X axis denotes T cell group, while the Y axis quantifies BATDA release as relative fluorescence units (RFUs) as detected by a time-resolved fluorometer. Horizontal lines represent means. ns=not significant, ****p<0.0001.

FIG. 35A depicts the mean percentage of PBNKs expressing degranulation marker CD107a (Y axis) following overnight co-culture at an ET ratio of 1:1 with wild-type iNK cells (“+WT”), B2M KO iNK cells (“+ B2M KO”), or B2M KO iNK cells expressing transgenic HLA-E with a fused HLA-G signal peptide sequence comprising VMAPRTLIL (SEQ ID NO: 1236) (“+1737”) or VMAPRTLVL (SEQ ID NO: 1238) (“+1738”). PBNKs cultured alone (PBNK alone) were included as a control. These data indicate HLA-E expression protects B2M KO iNK cells from PBNK cytotoxicity. Representative data collated from 3 donors in duplicate (N=6); error bars represent standard deviation (SD); *p<0.05, ***p<0.001, ****p<0.0001 by one-way ANOVA.

FIG. 35B depicts the mean percent lysis of WT iNK cells or B2M KO iNK cells (Y axis) following overnight co-culture with PBNKs across various E:T ratios (X axis). Representative data collated from 3 donors in duplicate (N=6); error bars represent standard deviation (SD).

FIG. 35C depicts the mean percent lysis of B2M KO iNK cells or B2M KO/HLA-E KI INK cells (“1737”) (Y axis) following overnight co-culture with PBNKs across various E:T ratios (X axis). HLA-E KI comprised a fused HLA-G signal peptide sequence comprising VMAPRTLIL (SEQ ID NO: 1236). Representative data collated from 3 donors in duplicate (N=6); error bars represent standard deviation (SD).

FIG. 35D depicts the mean percent lysis of B2M KO iNK cells or B2M KO/HLA-E KI INK cells (“1738”) (Y axis) following overnight co-culture with PBNKs across various E:T ratios (X axis). HLA-E KI comprised a fused HLA-G signal peptide sequence comprising VMAPRTLVL (SEQ ID NO: 1238). Representative data collated from 3 donors in duplicate (N=6); error bars represent standard deviation (SD).

### DETAILED DESCRIPTION

Some aspects of the disclosure are based, at least in part, on the recognition that certain genomic modifications of cells (e.g., pluripotent stem cells, e.g., cells differentiated from edited pluripotent stem cells and/or progeny of such cells) result in prevention of immune rejection and/or improved persistence. The present disclosure encompasses such genomically edited cells, compositions comprising such genomically edited cells, as well as methods of manufacturing and methods of using such genomically edited cells (e.g., to treat one or more disorder described herein).

### Definitions and Abbreviations

Unless otherwise specified, each of the following terms have the meaning set forth in this section.

The indefinite articles “a” and “an” refer to at least one of the associated noun, and are used interchangeably with the terms “at least one” and “one or more.” The conjunctions “or” and “and/or” are used interchangeably as non-exclusive disjunctions.

The term “cancer” (also used interchangeably with the terms, “hyperproliferative” and “neoplastic”), as used herein, refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. In some embodiments, “cancer” includes malignancies of or affecting various organ systems, such as lung, breast, thyroid, lymphoid, gastrointestinal, and genito-urinary tract. In some embodiments, “cancer” includes adenocarcinomas which include malignancies such as most colon cancers, renal-cell carcinoma, prostate cancer and/or testicular tumors, non-small cell carcinoma of the lung, cancer of the small intestine and/or cancer of the esophagus.

As used herein, the term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term carcinoma, as used herein, is well-recognized in the art. Exemplary carcinomas include those forming from tissue of the cervix, lung, prostate, breast, head and neck, colon and ovary. In some embodiments, carcinoma also includes carcinosarcomas, e.g., which include malignant tumors composed of carcinomatous and sarcomatous tissues. In some embodiments, an “adenocarcinoma” is a carcinoma derived from glandular tissue or in which the tumor cells form recognizable glandular structures. In some embodiments, a “sarcoma” is art recognized and refers to malignant tumors of mesenchymal derivation.

The terms “CRISPR/Cas nuclease” as used herein refer to any CRISPR/Cas protein with DNA nuclease activity, e.g., a Cas9 or a Cas 12 protein that exhibits specific association (or “targeting”) to a DNA target site, e.g., within a genomic sequence in a cell in the presence of a guide molecule. The strategies, systems, and methods disclosed herein can use any combination of CRISPR/Cas nuclease disclosed herein, or known to those of ordinary skill in the art. Those of ordinary skill in the art will be aware of additional CRISPR/Cas nucleases and variants suitable for use in the context of the present disclosure, and it will be understood that the present disclosure is not limited in this respect.

The term “differentiation” as used herein is the process by which an unspecialized (“uncommitted”) or less specialized cell acquires the features of a specialized cell such as, for example, a blood cell or a muscle cell. In some embodiments, a differentiated or differentiation-induced cell is one that has taken on a more specialized (“committed”) position within the lineage of a cell. For example, an iPSC can be differentiated into various more differentiated cell types, for example, a neural or a hematopoietic stem cell, a lymphocyte, a cardiomyocyte, and other cell types, upon treatment with suitable differentiation factors in the cell culture medium. In some embodiments, suitable methods, differentiation factors, and cell culture media for the differentiation of pluri- and multipotent cell types into more differentiated cell types are well known to those of skill in the art. In some embodiments, the term “committed”, is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type.

The terms “differentiation marker.” “differentiation marker gene,” or “differentiation gene,” as used herein refers to genes or proteins whose expression are indicative of cell differentiation occurring within a cell, such as a pluripotent cell. In some embodiments, differentiation marker genes include, but are not limited to, the following genes: CD34, CD4, CD8, CD3, CD56 (NCAM), CD49, CD45, NK cell receptor (cluster of differentiation 16 (CD16)), natural killer group-2 member D (NKG2D), CD69, NKp30, NKp44, NKp46, CD158b, FOXA2, FGF5, SOX17, XIST, NODAL, COL3A1, OTX2, DUSP6, EOMES, NR2F2, NROB1, CXCR4, CYP2B6, GAT A3, GATA4, ERBB4, GATA6, HOXC6, INHA, SMAD6, RORA, NIPBL, TNFSF11, CDH11, ZIC4, GAL, SOX3, PITX2, APOA2, CXCL5, CER1, FOXQ1, MLL5, DPP10, GSC, PCDH10, CTCFL, PCDH20, TSHZ1, MEGF10, MYC, DKK1, BMP2, LEFTY2, HES1, CDX2, GNAS, EGR1, COL3A1, TCF4, HEPH, KDR, TOX, FOXA1, LCK, PCDH7, CD1D FOXG1, LEFTY1, TUJ1, T gene (Brachyury), ZIC1, GATA1, GATA2, HDAC4, HDAC5, HDAC7, HDAC9, NOTCH1, NOTCH2, NOTCH4, PAX5, RBPJ, RUNX1, STAT1 and STAT3.

The terms “differentiation marker gene profile,” or “differentiation gene profile.” “differentiation gene expression profile,” “differentiation gene expression signature,” “differentiation gene expression panel.” “differentiation gene panel,” or “differentiation gene signature” as used herein refer to expression or levels of expression of a plurality of differentiation marker genes.

The term “edited iNK cell” as used herein refers to an induced pluripotent stem cell (iPSC)-derived natural killer (iNK) cell which has been modified to change at least one expression product of at least one gene at some point in the development of the cell. In some embodiments, a modification can be introduced using, e.g., gene editing techniques such as CRISPR-Cas or, e.g., dominant-negative constructs. In some embodiments, an iNK cell is edited at a time point before it has differentiated into an iNK cell, e.g., at a precursor stage, at a stem cell stage, etc. In some embodiments, an edited iNK cell is compared to a non-edited iNK cell (an NK cell produced by differentiating an iPSC cell, which iPSC cell and/or iNK cell do not have modifications, e.g., genetic modifications).

The term “embryonic stem cell” as used herein refers to pluripotent stem cells derived from the inner cell mass of the embryonic blastocyst. In some embodiments, embryonic stem cells are pluripotent and give rise during development to all derivatives of the three primary germ layers: ectoderm, endoderm and mesoderm. In some such embodiments, embryonic stem cells do not contribute to the extra-embryonic membranes or the placenta, i.e., are not totipotent.

The term “endogenous,” as used herein in the context of nucleic acids (e.g., genes, protein-encoding genomic regions, promoters), refers to a native nucleic acid or protein in its natural location, e.g., within the genome of a cell.

The term “essential gene” as used herein with respect to a cell refers to a gene that encodes at least one gene product that is required for survival, proliferation, development, and/or differentiation of the cell. An essential gene can be a housekeeping gene that is essential for survival of all cell types or a gene that is required to be expressed in a specific cell type for survival, proliferation, and development under particular culture conditions, e.g., for proper differentiation of iPS or ES cells or expansion of iPS- or ES-derived cells. Loss of function of an essential gene results, in some embodiments, in a significant reduction of cell survival, e.g., of the time a cell characterized by a loss of function of an essential gene survives as compared to a cell of the same cell type but without a loss of function of the same essential gene. In some embodiments, loss of function of an essential gene results in the death of the affected cell. In some embodiments, loss of function of an essential gene results in a significant reduction of cell proliferation, e.g., in the ability of a cell to divide, which can manifest in a significant time period the cell requires to complete a cell cycle, or, in some preferred embodiments, in a loss of a cell's ability to complete a cell cycle, and thus to proliferate at all.

The term “exogenous,” as used herein in the context of nucleic acids, e.g., expression constructs, cDNAs, indels, and nucleic acid vectors, refers to nucleic acids that have artificially been introduced into the genome of a cell using, for example, gene-editing or genetic engineering techniques, e.g., CRISPR-based editing techniques.

The term “genome editing system” refers to any system having DNA editing activity, e.g., RNA-guided DNA editing activity.

The terms “guide RNA” and “gRNA” refer to any nucleic acid that promotes the specific association (or “targeting”) of an RNA-guided nuclease such as a Cas9 or a Cpf1 (Cas12a) to a target sequence such as a genomic or episomal sequence in a cell.

The terms “hematopoietic stem cell,” or “definitive hematopoietic stem cell” as used herein, refer to CD34-positive stem cells. In some embodiments, CD34-positive stem cells are capable of giving rise to mature myeloid and/or lymphoid cell types. In some embodiments, the myeloid and/or lymphoid cell types include, for example, T cells, natural killer cells and/or B cells.

The terms “induced pluripotent stem cell” or “iPSC” as used herein to refer to a stem cell obtained from a differentiated somatic (e.g., adult, neonatal, or fetal) cell by a process referred to as reprogramming (e.g., dedifferentiation). In some embodiments, reprogrammed cells are capable of differentiating into tissues of all three germ or dermal layers: mesoderm, endoderm, and ectoderm. iPSCs are not found in nature.

The term “multipotent stem cell” as used herein refers to a cell that has the developmental potential to differentiate into cells of one or more germ layers (ectoderm, mesoderm and endoderm), but not all three germ layers. Thus, in some embodiments, a multipotent cell may also be termed a “partially differentiated cell.” Multipotent cells are well-known in the art, and examples of multipotent cells include adult stem cells, such as for example, hematopoietic stem cells and neural stem cells. In some embodiments, “multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, in some embodiments, “multipotency” refers to a state of a cell with a degree of developmental potential that is less than totipotent and pluripotent.

The term “nuclease” as used herein refers to any protein that catalyzes the cleavage of phosphodiester bonds. In some embodiments the nuclease is a DNA nuclease. In some embodiments the nuclease is a “nickase” which causes a single-strand break when it cleaves double-stranded DNA, e.g., genomic DNA in a cell. In some embodiments the nuclease causes a double-strand break when it cleaves double-stranded DNA, e.g., genomic DNA in a cell. In some embodiments the nuclease binds a specific target site within the double-stranded DNA that overlaps with or is adjacent to the location of the resulting break. In some embodiments, the nuclease causes a double-strand break that contains overhangs ranging from 0 (blunt ends) to 22 nucleotides in both 3′ and 5′ orientations. As discussed herein, CRISPR/Cas nucleases, zinc finger nucleases (ZFNs), transcription activator-like effector nucleases (TALENs) and meganucleases are exemplary nucleases that can be used in accordance with the strategies, systems, and methods of the present disclosure.

The term “pluripotent” as used herein refers to ability of a cell to form all lineages of the body or soma (i.e., the embryo proper) or a given organism (e.g., human). For example, embryonic stem cells are a type of pluripotent stem cells that are able to form cells from each of the three germ layers, the ectoderm, the mesoderm, and the endoderm. Generally, pluripotency may be described as a continuum of developmental potencies ranging from an incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell or an induced pluripotent stem cell).

The term “pluripotency” as used herein refers to a cell that has the developmental potential to differentiate into cells of all three germ layers (ectoderm, mesoderm, and endoderm). In some embodiments, pluripotency can be determined, in part, by assessing pluripotency characteristics of the cells. In some embodiments, pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

The term “pluripotent stem cell morphology” as used herein refers to the classical morphological features of an embryonic stem cell. In some embodiments, normal embryonic stem cell morphology is characterized as small and round in shape, with a high nucleus-to-cytoplasm ratio, the notable presence of nucleoli, and typical intercell spacing.

The term “polynucleotide” (including, but not limited to “nucleotide sequence”, “nucleic acid”, “nucleic acid molecule”, “nucleic acid sequence”, and “oligonucleotide”) as used herein refers to a series of nucleotide bases (also called “nucleotides”) in DNA and RNA, and means any chain of two or more nucleotides. In some embodiments, polynucleotides, nucleotide sequences, nucleic acids etc. can be chimeric mixtures or derivatives or modified versions thereof, single-stranded or double-stranded. In some such embodiments, modifications can occur at the base moiety, sugar moiety, or phosphate backbone, for example, to improve stability of the molecule, its hybridization parameters, etc. In general, a nucleotide sequence typically carries genetic information, including, but not limited to, the information used by cellular machinery to make proteins and enzymes. In some embodiments, a nucleotide sequence and/or genetic information comprises double- or single-stranded genomic DNA, RNA, any synthetic and genetically manipulated polynucleotide, and/or sense and/or antisense polynucleotides. In some embodiments, nucleic acids contain modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “potency” or “developmental potency” as used herein refers to the sum of all developmental options accessible to the cell (i.e., the developmental potency), particularly, for example in the context of cellular developmental potential. In some embodiments, the continuum of cell potency includes, but is not limited to, totipotent cells, pluripotent cells, multipotent cells, oligopotent cells, unipotent cells, and terminally differentiated cells.

The terms “prevent,” “preventing.” and “prevention” as used herein in the context of a disease refer to the prevention of the disease in a mammal, e.g., in a human, including (a) avoiding or precluding the disease; (b) affecting the predisposition toward the disease; or (c) preventing or delaying the onset of at least one symptom of the disease.

The terms “protein,” “peptide” and “polypeptide” as used herein are used interchangeably to refer to a sequential chain of amino acids linked together via peptide bonds. The terms include individual proteins, groups or complexes of proteins that associate together, as well as fragments or portions, variants, derivatives and analogs of such proteins. Unless otherwise specified, peptide sequences are presented herein using conventional notation, beginning with the amino or N-terminus on the left, and proceeding to the carboxyl or C-terminus on the right. Standard one-letter or three-letter abbreviations can be used.

The terms “reprogramming” or “dedifferentiation” or “increasing cell potency” or “increasing developmental potency” as used herein refer to a method of increasing potency of a cell or dedifferentiating a cell to a less differentiated state. For example, in some embodiments, a cell that has an increased cell potency has more developmental plasticity (i.e., can differentiate into more cell types) compared to the same cell in the non-reprogrammed state. That is, in some embodiments, a reprogrammed cell is one that is in a less differentiated state than the same cell in a non-reprogrammed state. In some embodiments, “reprogramming” refers to de-differentiating a somatic cell, or a multipotent stem cell, into a pluripotent stem cell, also referred to as an induced pluripotent stem cell, or iPSC. Suitable methods for the generation of iPSCs from somatic or multipotent stem cells are well known to those of skill in the art.

The terms “RNA-guided nuclease” and “RNA-guided nuclease molecule” are used interchangeably herein. In some embodiments, the RNA-guided nuclease is a RNA-guided DNA endonuclease enzyme. In some embodiments, the RNA-guided nuclease is a CRISPR nuclease. Non-limiting examples of RNA-guided nucleases are listed in Table 2 below, and the methods and compositions disclosed herein can use any combination of RNA-guided nucleases disclosed herein, or known to those of ordinary skill in the art. Those of ordinary skill in the art will be aware of additional nucleases and nuclease variants suitable for use in the context of the present disclosure, and it will be understood that the present disclosure is not limited in this respect.

TABLE 2
RNA-Guided Nucleases

| | Length | | |
|---|---|---|---|
| Nuclease | (a.a.) | PAM | Reference |
| SpCas9 | 1368 | NGG | Cong et al., Science. 2013; 339(6121): 819-23 |
| SaCas9 | 1053 | NNGRRT | Ran et al., Nature. 2015; 520(7546): 186-91. |
| (KKH) | 1067 | NNNRRT | Kleinstiver et al., Nat Biotechnol. |
| SaCas9 | | | 2015; 33(12): 1293-1298 |
| AsCpf1 | 1353 | TTTV | Zetsche et al., Nat Biotechnol. 2017; 35(1): 31-34. |
| (AsCas12a) | | | |
| LbCpf1 | 1274 | TTTV | Zetsche et al., Cell. 2015; 163(3): 759-71. |
| (LbCas12a) | | | |
| CasX | 980 | TTC | Burstein et al., Nature. 2017; 542(7640): 237-241. |
| CasY | 1200 | TA | Burstein et al., Nature. 2017; 542(7640): 237-241. |
| Cas12h1 | 870 | RTR | Yan et al., Science. 2019; 363(6422): 88-91. |
| Cas12i1 | 1093 | TTN | Yan et al., Science. 2019; 363(6422): 88-91. |
| Cas12c1 | unknown | TG | Yan et al., Science. 2019; 363(6422): 88-91. |
| Cas12c2 | unknown | TN | Yan et al., Science. 2019; 363(6422): 88-91. |
| eSpCas9 | 1423 | NGG | Chen et al., Nature. 2017; 550(7676): 407-410. |
| Cas9-HF1 | 1367 | NGG | Chen et al., Nature. 2017; 550(7676): 407-410. |
| HypaCas9 | 1404 | NGG | Chen et al., Nature. 2017; 550(7676): 407-410. |
| dCas9-Fokl | 1623 | NGG | U.S. Pat. No. 9,322,037 |
| Sniper-Cas9 | 1389 | NGG | Lee et al., Nat Commun. 2018; 9(1): 3048. |
| xCas9 | 1786 | NGG, NG, | Wang et al., Plant Biotechnol J. 2018; pbi.13053. |
| | | GAA, GAT | |
| AaCas12b | 1129 | TTN | Teng et al. Cell Discov. 2018; 4: 63. |
| evoCas9 | 1423 | NGG | Casini et al., Nat Biotechnol. 2018; 36(3): 265-271. |
| SpCas9-NG | 1423 | NG | Nishimasu et al., Science. 2018; 361(6408): 1259-1262. |
| VRQR | 1368 | NGA | Li et al., The CRISPR Journal, 2018; 01: 01 |
| VRER | 1372 | NGCG | Kleinstiver et al., Nature. 2016; 529(7587): 490-5. |
| NmeCas9 | 1082 | NNNNGATT | Amrani et al., Genome Biol. 2018; 19(1): 214. |
| CjCas9 | 984 | NNNNRYAC | Kim et al., Nat Commun. 2017; 8: 14500. |
| BhCas12b | 1108 | ATTN | Strecker et al., Nat Commun. 2019 Jan. 22; 10(1): 212. |
| BhCas12bV4 | 1108 | ATTN | Strecker et al., Nat Commun. 2019 Jan. 22; 10(1): 212. |
| CasΦ | 700-800 | TBN | Pausch et al., Science 2020; 369(6501): 333-337. |
| | | (where B is | |
| | | G, T, or C) | |

Additional suitable RNA-guided nucleases, e.g., Cas9 and Cas 12 nucleases, will be apparent to the skilled artisan in view of the present disclosure, and the disclosure is not limited by the exemplary suitable nucleases provided herein. In some embodiments, a suitable nuclease is a Cas9 or Cpf1 (Cas12a) nuclease. In some embodiments, the disclosure also embraces nuclease variants, e.g., Cas9 or Cpf1 nuclease variants. In some embodiments, a nuclease is a nuclease variant, which refers to a nuclease comprising an amino acid sequence characterized by one or more amino acid substitutions, deletions, or additions as compared to the wild type amino acid sequence of the nuclease. In some embodiments, a suitable nuclease and/or nuclease variant may also include purification tags (e.g., polyhistidine tags) and/or signaling peptides, e.g., comprising or consisting of a nuclear localization signal sequence. Some non-limiting examples of suitable nucleases and nuclease variants are described in more detail elsewhere herein and also include those described in PCT application PCT/US2019/22374, filed Mar. 14, 2019, and entitled “Systems and Methods for the Treatment of Hemoglobinopathies,” the entire contents of which are incorporated herein by reference. In some embodiments, the RNA-guided nuclease is an Acidaminococcus sp. Cpf1 variant (AsCpf1 variant). In some embodiments, suitable Cpf1 nuclease variants, including suitable AsCpf1 variants will be known or apparent to those of ordinary skill in the art based on the present disclosure, and include, but are not limited to, the Cpf1 variants disclosed herein or otherwise known in the art. For example, in some embodiments, the RNA-guided nuclease is a Acidaminococcus sp. Cpf1 RR variant (AsCpf1-RR). In another embodiment, the RNA-guided nuclease is a Cpf1 RVR variant. For example, suitable Cpf1 variants include those having an M537R substitution, an H800A substitution, and/or an F870L substitution, or any combination thereof (numbering scheme according to AsCpf1 wild-type sequence).

The term “subject” as used herein means a human or non-human animal. In some embodiments a human subject can be any age (e.g., a fetus, infant, child, young adult, or adult). In some embodiments a human subject may be at risk of or suffer from a disease, or may be in need of alteration of a gene or a combination of specific genes. Alternatively, in some embodiments, a subject may be a non-human animal, which may include, but is not limited to, a mammal. In some embodiments, a non-human animal is a non-human primate, a rodent (e.g., a mouse, rat, hamster, guinea pig, etc.), a rabbit, a dog, a cat, and so on. In certain embodiments of this disclosure, the non-human animal subject is livestock, e.g., a cow, a horse, a sheep, a goat, etc. In certain embodiments, the non-human animal subject is poultry, e.g., a chicken, a turkey, a duck, etc.

The terms “treatment,” “treat,” and “treating,” as used herein refer to a clinical intervention aimed to reverse, alleviate, delay the onset of, or inhibit the progress, ameliorate, reduce severity of, prevent or delay the recurrence of a disease, disorder, or condition or one or more symptoms thereof, and/or improve one or more symptoms of a disease, disorder, or condition as described herein. In some embodiments, a condition includes an injury. In some embodiments, an injury may be acute or chronic (e.g., tissue damage from an underlying disease or disorder that causes, e.g., secondary damage such as tissue injury). In some embodiments, treatment, e.g., in the form of a modified NK cell or a population of modified NK cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, in some embodiments, treatment may be administered to a susceptible individual prior to the onset of symptoms (e.g., in light of genetic or other susceptibility factors). In some embodiments, treatment may also be continued after symptoms have resolved, for example to prevent or delay their recurrence. In some embodiments, treatment results in improvement and/or resolution of one or more symptoms of a disease, disorder or condition.

The term “variant” as used herein refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As used herein, the term “functional variant” refers to a variant that confers the same function as the reference entity, e.g., a functional variant of a gene product of an essential gene is a variant that promotes the survival and/or proliferation of a cell. It is to be understood that a functional variant need not be functionally equivalent to the reference entity as long as it confers the same function as the reference entity.

### Stem Cells

Methods of the disclosure can be used to culture stem cells. Stem cells are typically cells that have the capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cells (USSCs). Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation.

Pluripotent stem cells are generally known in the art. The present disclosure provides, in part, technologies (e.g., systems, compositions, methods, etc.) related to pluripotent stem cells. In some embodiments, pluripotent stem cells are stem cells that: (a) are capable of inducing teratomas when transplanted in immunodeficient (SCID) mice; (b) are capable of differentiating to cell types of all three germ layers (e.g., can differentiate to ectodermal, mesodermal, and endodermal cell types); and/or (c) express one or more markers of embryonic stem cells (e.g., human embryonic stem cells express October 4, alkaline phosphatase, SSEA-3 surface antigen, SSEA-4 surface antigen, nanog, TRA-1-60, TRA-1-81. SOX2, REX1, etc.). In some aspects, human pluripotent stem cells do not show expression of differentiation markers. In some embodiments, ES cells and/or iPSCs cultured using methods of the disclosure maintain their pluripotency (e.g., (a) are capable of inducing teratomas when transplanted in immunodeficient (SCID) mice; (b) are capable of differentiating to cell types of all three germ layers (e.g., can differentiate to ectodermal, mesodermal, and endodermal cell types); and/or (c) express one or more markers of embryonic stem cells).

In some embodiments, ES cells (e.g., human ES cells) can be derived from the inner cell mass of blastocysts or morulae. In some embodiments, ES cells can be isolated from one or more blastomeres of an embryo, e.g., without destroying the remainder of the embryo. In some embodiments, ES cells can be produced by somatic cell nuclear transfer. In some embodiments, ES cells can be derived from fertilization of an egg cell with sperm or DNA, nuclear transfer, parthenogenesis, or by means to generate ES cells, e.g., with homozygosity in the HLA region. In some embodiments, human ES cells can be produced or derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce an embryonic cell. Exemplary human ES cells are known in the art and include, but are not limited to, MAO1, MAO9, ACT-4, No. 3, H1, H7, H9, H14 and ACT30 ES cells. In some embodiments, human ES cells, regardless of their source or the particular method used to produce them, can be identified based on, e.g., (i) the ability to differentiate into cells of all three germ layers, (ii) expression of at least Oct-4 and alkaline phosphatase, and/or (iii) ability to produce teratomas when transplanted into immunocompromised animals. In some embodiments, ES cells have been serially passaged as cell lines.

iPSCs

Induced pluripotent stem cells (iPSC) are a type of pluripotent stem cell artificially derived from a non-pluripotent cell, such as an adult somatic cell (e.g., a fibroblast cell or other suitable somatic cell), by inducing expression of certain genes. iPSCs can be derived from any organism, such as a mammal. In some embodiments, iPSCs are produced from mice, rats, rabbits, guinea pigs, goats, pigs, cows, non-human primates or humans. iPSCs are similar to ES cells in many respects, such as the expression of certain stem cell genes and proteins, chromatin methylation patterns, doubling time, embryoid body formation, teratoma formation, viable chimera formation, potency and/or differentiability. Various suitable methods for producing iPSCs are known in the art. In some embodiments, iPSCs can be derived by transfection of certain stem cell-associated genes (such asOct-3/4 (Pouf51) and Sox2) into non-pluripotent cells, such as adult fibroblasts. Transfection can be achieved through viral vectors, such as retroviruses, lentiviruses, or adenoviruses. Additional suitable reprogramming methods include the use of vectors that do not integrate into the genome of the host cell, e.g., episomal vectors, or the delivery of reprogramming factors directly via encoding RNA or as proteins has also been described. For example, cells can be transfected with Oct3/4, Sox2, Klf4, and/or c-Myc using a retroviral system or with OCT4, SOX2, NANOG, and/or LIN28 using a lentiviral system. After 3-4 weeks, small numbers of transfected cells begin to become morphologically and biochemically similar to pluripotent stem cells, and can be isolated through morphological selection, doubling time, or through a reporter gene and antibiotic selection. In one example, iPSCs from adult human cells are generated by the method described by Yu et al. (Science 318(5854): 1224 (2007)) or Takahashi et al. (Cell 131:861-72 (2007)). In some embodiments, iPSCs are generated by a commercial source. In some embodiments, iPSCs are generated by a vendor. In some embodiments, iPSCs are generated by a contract research organization. Numerous suitable methods for reprogramming are known to those of skill in the art, and the present disclosure is not limited in this respect.

### Genetically Engineered Stem Cells

In some embodiments, a stem cell (e.g., iPSC) described herein is genetically engineered to introduce a disruption in one or more targets described herein. For example, in some embodiments, a stem cell (e.g., iPSC) can be genetically engineered to knockout all or a portion of one or more target genes, introduce a frameshift in one or more target genes, and/or cause a truncation of an encoded gene product (e.g., by introducing a premature stop codon). In some embodiments, a stem cell (e.g., iPSC) can be genetically engineered to knockout all or a portion of a target gene using a gene-editing system, e.g., as described herein. In some such embodiments, a gene-editing system may be or comprise a CRISPR system, a zinc finger nuclease system, a TALEN, and/or a meganuclease.

### TGF Signaling

In certain embodiments, the disclosure provides a genetically engineered stem cell, and/or progeny cell comprising a disruption in TGF signaling, e.g., TGF beta signaling. This is useful, for example, in circumstances where it is desirable to generate a differentiated cell from pluripotent stem cell, wherein TGF signaling, e.g., TGF beta signaling is disrupted in the differentiated cell.

For example, TGF beta signaling inhibits or decreases the survival and/or activity of some differentiated cell types that are useful for therapeutic applications, e.g., TGF beta signaling is a negative regulator of natural killer cells, which can be used in immunotherapeutic applications. In some embodiments, it is desirable to generate a clinically effective number of natural killer cells comprising a genetic modification that disrupts TGF beta signaling, thus avoiding the negative effect of TGF beta on the clinical effectiveness of such cells. It is advantageous, in some embodiments, to source such NK cells from a pluripotent stem cell, instead of, for example, from mature NK cells obtained from a donor. Modifying the stem cell instead of the differentiated cell has, among others, the advantage of allowing for clonal derivation, characterization, and/or expansion of a specific genotype, e.g., a specific stem cell clone harboring a specific genetic modification (e.g., a targeted disruption of TGFβRII in the absence of any undesired (e.g., off-target) modifications). In some embodiments, the stem cell, e.g., the human iPSC, is genetically engineered not to express one or more TGFβ receptor, e.g., TGFβRII, or to express a dominant negative variant of a TGFβ receptor, e.g., a dominant negative TGFβRII variant. Exemplary sequences of TGFβRII are set forth in KR710923.1. NM_001024847.2, and NM_003242.5. An exemplary dominant negative TGFβRII is disclosed in Immunity. 2000 February; 12(2):171-81.

### Additional Loss-of-Function Modifications

In certain embodiments, the disclosure provides a genetically engineered stem cell, and/or progeny cell, that additionally or alternatively comprises a disruption in interleukin signaling, e.g., IL-15 signaling. IL-15 is a cytokine with structural similarity to Interleukin-2 (IL-2), which binds to and signals through a complex composed of IL-2/IL-15 receptor beta chain (CD122) and the common gamma chain (gamma-C, CD132). Exemplary sequences of IL-15 are provided in NG_029605.2. Disruption of IL-15 signaling may be useful, for example, in circumstances where it is desirable to generate a differentiated cell from a pluripotent stem cell, but with certain signaling pathways (e.g., IL-15) disrupted in the differentiated cell. IL-15 signaling can inhibit or decrease survival and/or activity of some types of differentiated cells, such as cells that may be useful for therapeutic applications. For example, IL-15 signaling is a negative regulator of natural killer (NK) cells. CISH (encoded by the CISH gene) is downstream of the IL-15 receptor and can act as a negative regulator of IL-15 signaling in NK cells.

As used herein, the term “CISH” refers to the Cytokine Inducible SH2 Containing Protein (see, e.g., Delconte et al., Nat Immunol. 2016 July; 17(7):816-24; exemplary sequences for CISH are set forth as NG_023194.1). In some embodiments, disruption of CISH regulation may increase activation of Jak/STAT pathways, leading to increased survival, proliferation and/or effector functions of NK cells. Thus, in some embodiments, genetically engineered NK cells (e.g., iNK cells, e.g., generated from genetically engineered hiPSCs comprising a disruption of CISH regulation) exhibit greater responsiveness to IL-15-mediated signaling than non-genetically engineered NK cells. In some such embodiments, genetically engineered NK cells exhibit greater effector function relative to non-genetically engineered NK cells.

In some embodiments, a genetically engineered stem cell and/or progeny cell, additionally or alternatively, comprises a disruption and/or loss of function in one or more of B2M, NKG2A, PD1. TIGIT, ADORA2a, CIITA, HLA class II histocompatibility antigen alpha chain genes, HLA class II histocompatibility antigen beta chain genes, CD32B, or TRAC.

As used herein, the term “B2M” (B2 microglobulin) refers to a serum protein found in association with the major histocompatibility complex (MHC) class I heavy chain on the surface of nearly all nucleated cells. Exemplary sequences for B2M are set forth as NG_012920.2.

B2M amino acid sequence
SEQ ID NO: 1241
MSRSVALAVLALLSLSGLEAIQRTPKIQVYSRHPAE
NGKSNFLNCYVSGFHPSDIEVDLLKNGERIEKVEHS
DLSFSKDWSFYLLYYTEFTPTEKDEYACRVNHVTLS
QPKIVKWDRDM

As used herein, the term “NKG2A” (natural killer group 2A) refers to a protein belonging to the killer cell lectin-like receptor family, also called NKG2 family, which is a group of transmembrane proteins preferentially expressed in NK cells. This family of proteins is characterized by the type II membrane orientation and the presence of a C-type lectin domain. See, e.g., Kamiya-T et al., J Clin Invest 2019 https://doi.org/10.1172/JCI123955. Exemplary sequences for NKG2A are set forth as AF461812.1.

As used herein, the term “PD1” (Programmed cell death protein 1), also known CD279 (cluster of differentiation 279), refers to a protein found on the surface of cells that has a role in regulating the immune system's response to the cells of the human body by down-regulating the immune system and promoting self-tolerance by suppressing T cell inflammatory activity. PD1 is an immune checkpoint and guards against autoimmunity. Exemplary sequences for PD1 are set forth as NM_005018.3.

As used herein, the term “TIGIT” (T cell immunoreceptor with Ig and ITIM domains) refers to a member of the PVR (poliovirus receptor) family of immunoglobulin proteins. The product of this gene is expressed on several classes of T cells including follicular B helper T cells (TFH). Exemplary sequences for TIGIT are set forth in NM_173799.4.

As used herein, the term “ADORA2A” refers to the adenosine A2a receptor, a member of the guanine nucleotide-binding protein (G protein)-coupled receptor (GPCR) superfamily, which is subdivided into classes and subtypes. This protein, an adenosine receptor of A2A subtype, uses adenosine as the preferred endogenous agonist and preferentially interacts with the G(s) and G(olf) family of G proteins to increase intracellular CAMP levels. Exemplary sequences of ADORA2a are provided in NG_052804.1.

As used herein, the term “CIITA” refers to the protein located in the nucleus that acts as a positive regulator of class II major histocompatibility complex gene transcription, and is referred to as the “master control factor” for the expression of these genes. The protein also binds GTP and uses GTP binding to facilitate its own transport into the nucleus. Mutations in this gene have been associated with bare lymphocyte syndrome type II (also known as hereditary MHC class II deficiency or HLA class II-deficient combined immunodeficiency), increased susceptibility to rheumatoid arthritis, multiple sclerosis, and possibly myocardial infarction. See, e.g., Chang et al., J Exp Med 180:1367-1374; and Chang et al., Immunity. 1996 February; 4(2):167-78, the entire contents of each of which are incorporated by reference herein. An exemplary sequence of CIITA is set forth as NG_009628.1.

In some embodiments, two or more HLA class II histocompatibility antigen alpha chain genes and/or two or more HLA class II histocompatibility antigen beta chain genes are disrupted, e.g., knocked out, e.g., by genomic editing. For example, in some embodiments, two or more HLA class II histocompatibility antigen alpha chain genes selected from HLA-DQA1, HLA-DRA, HLA-DPA1, HLA-DMA, HLA-DQA2, and HLA-DOA are disrupted, e.g., knocked out. For another example, in some embodiments, two or more HLA class II histocompatibility antigen beta chain genes selected from HLA-DMB, HLA-DOB, HLA-DPB1, HLA-DQB1, HLA-DQB3, HLA-DQB2, HLA-DRB1, HLA-DRB3, HLA-DRB4, and HLA-DRB5 are disrupted, e.g., knocked out. See, e.g., Crivello et al., J Immunol January 2019, ji1800257; DOI: https://doi.org/10.4049/jimmunol.1800257, the entire contents of which are incorporated herein by reference.

As used herein, the term “CD32B” (cluster of differentiation 32B) refers to a low affinity immunoglobulin gamma Fc region receptor II-b protein that, in humans, is encoded by the FCGR2B gene. See, e.g., Rankin-C T et al., Blood 2006 108(7):2384-91, the entire contents of which are incorporated herein by reference.

As used herein, the term “TRAC” refers to the T-cell receptor alpha subunit (constant), encoded by the TRAC locus.

### Gain-of-Function Modifications

In some embodiments, a target cell described herein (e.g., a stem cell (e.g., iPSC) described herein) can additionally be genetically engineered to comprise a genetic modification that leads to expression of one or more gene products of interest described herein using, e.g., a gene-editing system, e.g., as described herein. In some such embodiments, a gene-editing system may be or comprise a CRISPR system, a zinc finger nuclease system, a TALEN, and/or a meganuclease.

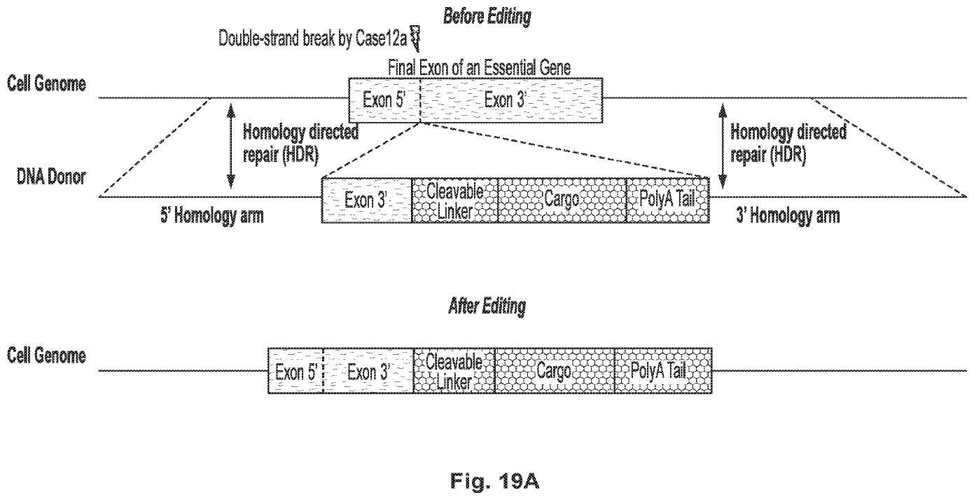

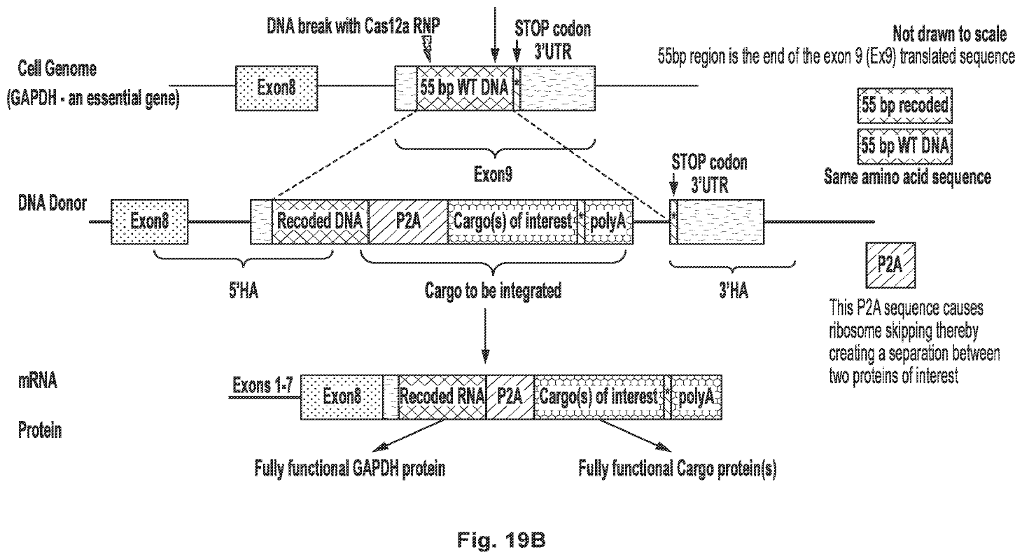

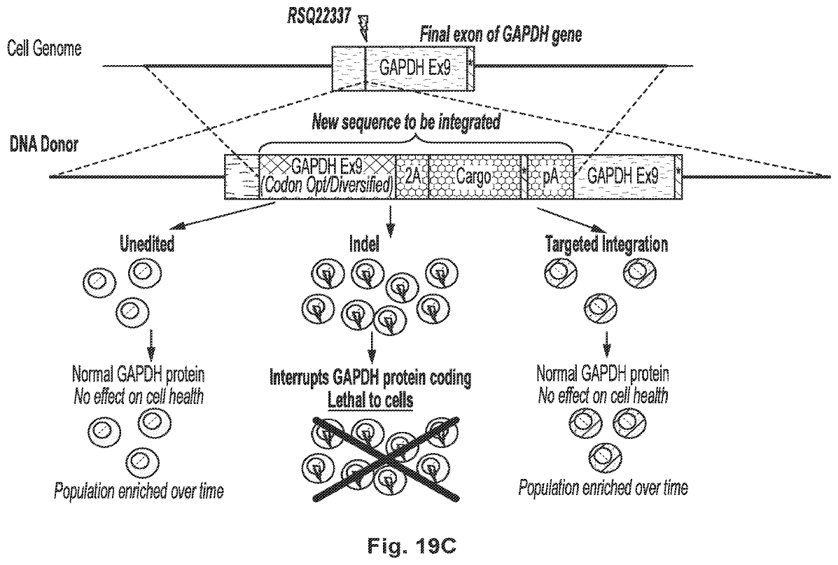

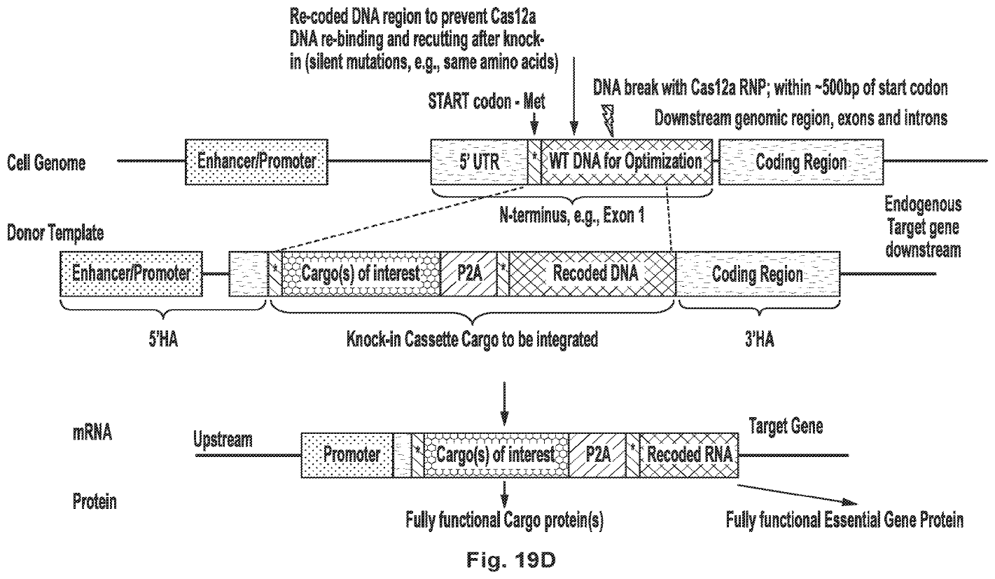

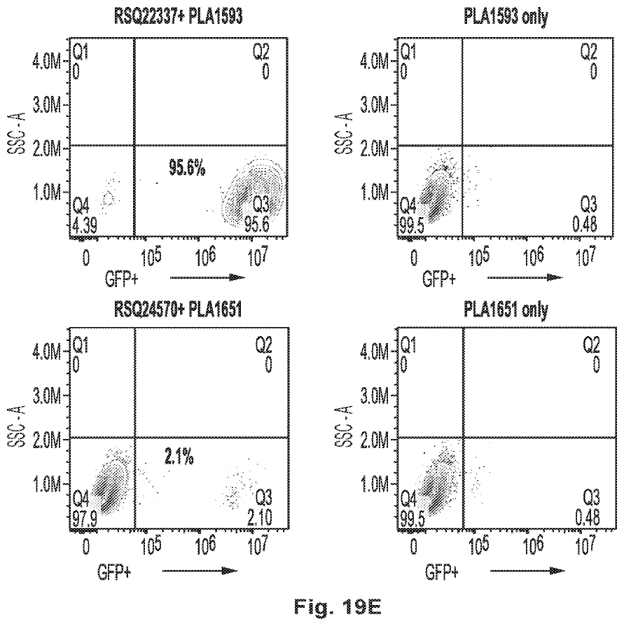

In some embodiments, a cell is produced by a method that comprises contacting the cell with a nuclease that causes a break within an endogenous coding sequence of an essential gene in the cell wherein the essential gene encodes at least one gene product that is required for survival and/or proliferation of the cell. The cell is also contacted with a donor template that comprises a knock-in cassette comprising an exogenous coding sequence for a gene product of interest in frame with and downstream (3′) or upstream (5′) of an exogenous coding sequence or partial coding sequence of the essential gene. The knock-in cassette is integrated into the genome of the cell by homology-directed repair (HDR) of the break, resulting in a genome-edited cell that expresses the gene product of interest and the gene product encoded by the essential gene that is required for survival and/or proliferation of the cell, or a functional variant thereof (e.g., as is illustrated in FIG. 19A-19D).

In some embodiments, the cell comprises a genome with an exogenous coding sequence for a gene product of interest in frame with and downstream (3′) of a coding sequence of an essential gene, wherein the essential gene encodes a gene product that is required for survival and/or proliferation of the cell.

In some embodiments, the cell comprises a genome with an exogenous coding sequence for a gene product of interest in frame with and upstream (5′) of a coding sequence of an essential gene, wherein the essential gene encodes a gene product that is required for survival and/or proliferation of the cell.

In some embodiments, the cell comprises a genomic modification, wherein the genomic modification comprises an insertion of an exogenous knock-in cassette within an endogenous coding sequence of an essential gene in the cell's genome, wherein the essential gene encodes a gene product that is required for survival and/or proliferation of the cell, wherein the knock-in cassette comprises an exogenous coding sequence for a gene product of interest in frame with and downstream (3′) of an exogenous coding sequence or partial coding sequence encoding the gene product of the essential gene, or a functional variant thereof, and wherein the cell expresses the gene product of interest and the gene product encoded by the essential gene that is required for survival and/or proliferation of the cell, or a functional variant thereof. In some embodiments, the gene product of interest and the gene product encoded by the essential gene are expressed from the endogenous promoter of the essential gene.

In one aspect, the present disclosure provides methods of editing the genome of a cell. In certain embodiments, the method comprises contacting the cell with a nuclease that causes a break within an endogenous coding sequence of an essential gene in the cell wherein the essential gene encodes at least one gene product that is required for survival, proliferation, and/or development of the cell. The cell is also contacted with (i) a donor template that comprises a knock-in cassette comprising an exogenous coding sequence for a gene product of interest in frame with and downstream (3′) of an exogenous coding sequence or partial coding sequence of the essential gene (FIG. 19B) and/or (ii) a donor template that comprises a knock-in cassette comprising an exogenous coding sequence for a gene product of interest in frame with and upstream (5′) of an exogenous coding sequence or partial coding sequence of the essential gene (FIG. 19D). The knock-in cassette is integrated into the genome of the cell by homology-directed repair (HDR) of the break, resulting in a genome-edited cell that expresses the gene product of interest and the gene product encoded by the essential gene that is required for survival, proliferation, and/or development of the cell, or a functional variant thereof. The genetically modified “knock-in” cell survives and proliferates to produce progeny cells with genomes that also include the exogenous coding sequence for the gene product of interest. This is illustrated in FIG. 19A for an exemplary method.

If the knock-in cassette is not properly integrated into the genome of the cell, undesired editing events that result from the break, e.g., NHEJ-mediated creation of indels, may produce a non-functional, e.g., out of frame, version of the essential gene. This produces a “knock-out” cell when the editing efficiency of the nuclease is high enough to disrupt both alleles. In certain embodiments, this produces a “knock-out” cell when the editing efficiency of the nuclease is high enough to disrupt one allele. Without sufficient functional copies of the essential gene these “knock-out” cells are unable to survive and do not produce any progeny cells.

Since the “knock-in” cells survive and the “knock-out” cells do not survive, the method automatically selects for the “knock-in” cells when it is applied to a population of starting cells. Significantly, in certain embodiments, the method does not require high knock-in efficiencies because of this automatic selection aspect. It is therefore particularly suitable for methods where the donor template is a dsDNA (e.g., a plasmid) where knock-in efficiencies are often below 5%. As noted in the exemplary method of FIG. 19C, in some embodiments some of the cells in the population of starting cells may remain unedited, i.e., unaffected by the nuclease. These cells would also survive and produce progeny with genomes that do not include the exogenous coding sequence for the gene product of interest. When the nuclease editing efficiency is high, e.g., about 60-90%, or higher the percentage of unedited cells will be relatively low as compared to the percentage of genetically modified cells. In some embodiments, high nuclease editing efficiencies (e.g., greater than 65%, greater than 70%, greater than 75%, greater than 80%, greater than 85%, greater than 90%, or greater than 95%) facilitates efficient population wide transgene integration, as the percentage of unedited cells will be relatively low as compared to the percentage of genetically modified cells. In some embodiments of the methods disclosed herein, at least about 65% of the cells (e.g., about 70%, about 75%, about 80%, about 85%, about 90%, about 91%, about 92%, about 93%, about 94%, about 95%, about 96%, about 97%, about 98%, or about 99% of the cells) are edited by a nuclease, e.g., but not limited to, a Cas12a or Cas9. In some embodiments, an RNP containing a CRISPR nuclease (e.g., Cas12a, Cas9, Cas12b, Cas12c, Cas12e, CasX, or CasΦ (Cas12j), or a variant thereof (e.g., a variant with a high editing efficiency), but not limited to) and a guide are capable of cleaving the locus of an essential gene (e.g., a terminal exon in the locus of any essential gene provided in Table 13) in at least 65% of the cells in a population of cells (e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the cells in a population of cells). In some embodiments, an RNP containing a CRISPR nuclease (e.g., Cas12a, Cas9, Cas12b, Cas12c, Cas12e, CasX, or CasΦ (Cas12j), or a variant thereof (e.g., a variant with a high editing efficiency), but not limited to) and a guide are capable of inducing transgene integration at a locus of an essential gene (e.g., a terminal exon in the locus of any essential gene provided in Table 13) in at least 65% of the cells in a population of cells (e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the cells in a population of cells), e.g., at between 4 days and 10 days (e.g., 4 days, 5 days, 6 days, 7 days, 8 days, 9 days or 10 days) after the cells in the population of cells is contacted with the RNP containing a CRISPR nuclease. In some embodiments, at least about 65% of the cells (e.g., about 70%, about 75%, about 80%, about 85%, about 90%, about 91%, about 92%, about 93%, about 94%, about 95%, about 96%, about 97%, about 98%, or about 99% of the cells) comprise an integrated transgene following editing, e.g., at between 4 and 10 days (e.g., 4 days, 5 days, 6 days, 7 days, 8 days, 9 days or 10 days) after the cells in the population of cells is contacted with the RNP containing a CRISPR nuclease and/or at least about 65% of the cells (e.g., about 70%, about 75%, about 80%, about 85%, about 90%, about 91%, about 92%, about 93%, about 94%, about 95%, about 96%, about 97%, about 98%, or about 99% of the cells) comprise a genomic edit that results in loss of function of a gene following editing, e.g., at between 4 and 10 days (e.g., 4 days, 5 days, 6 days, 7 days, 8 days, 9 days or 10 days) after the cells in the population of cells is contacted with the RNP containing a CRISPR nuclease. In some embodiments, editing efficiency is determined prior to target cell die off, e.g., at day 1 and/or day 2 post transfection or transduction. In some embodiments, editing efficiency measured at day 1 and/or day 2 post transfection or transduction may not capture the complete proportion of cells for which editing occurred, as in some embodiments, certain editing events may result in near immediate and/or swift cell death. In some embodiments, near immediate and/or swift cell death may be any period of time less than 48 hours post transfection or transduction, for example, less than 48 hours, less than 44 hours, less than 40 hours, less than 36 hours, less than 32 hours, less than 28 hours, less than 24 hours, less than 20 hours, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, or less than 1 hour after transfection or transduction.

In some embodiments, the nuclease causes a double-strand break. In some embodiments the nuclease causes a single-strand break, e.g., in some embodiments the nuclease is a nickase. In some embodiments the nuclease is a prime editor which comprises a nickase domain fused to a reverse transcriptase domain. In some embodiments the nuclease is an RNA-guided prime editor and the gRNA comprises the donor template. In some embodiments a dual-nickase system is used which causes a double-strand break via two single-strand breaks on opposing strands of a double-stranded DNA, e.g., genomic DNA of the cell.

In some embodiments, the present disclosure provides methods suitable for high-efficiency knock-in (e.g., a high proportion of a cell population comprises a knock-in allele), overcoming a major manufacturing challenge. Historically, gene of interest knock-in using plasmid vectors results in efficiencies typically between 0.1 and 5% (see e.g., Zhu et al., CRISPR/Cas-Mediated Selection-free Knockin Strategy in Human Embryonic Stem Cells. Stem Cell Reports. 2015; 4(6):1103-1111). This low knock-in efficiency can result in a need for extensive time and resources devoted to screening potentially edited clones.

In some embodiments, a gene of interest (e.g., a gene capable of bestowing a gain-of-function modification) knocked into a cell may have a role in effector function, specificity, stealth, persistence, homing/chemotaxis, and/or resistance to certain chemicals (see for example, Saetersmoen et al., Seminars in Immunopathology, 2019).

In certain embodiments, the present disclosure provides methods for creation of knock-in cells that maintain high levels of expression regardless of age, differentiation status, and/or exogenous conditions. For example, in some embodiments, an integrated cargo is expressed at an optimal level with a desired subcellular localization as a function of an insertion site. In some embodiments, the present disclosure provides such cells.

In some embodiments, a genetically engineered stem cell and/or progeny cell, additionally or alternatively, comprises a genetic modification that leads to expression of human leukocyte antigen G (HLA-G) and/or human leukocyte antigen E (HLA-E). In some embodiments, a genetically engineered stem cell and/or progeny cell, additionally or alternatively, comprises a genetic modification that leads to expression one or more of a CAR; a non-naturally occurring variant of FcγRIII (CD16); interleukin 15 (IL-15); an IL-15 receptor (IL-15R) agonist, or a constitutively active variant of an IL-15 receptor; interleukin 12 (IL-12); an IL-12 receptor (IL-12R) agonist, or a constitutively active variant of an IL-12 receptor; and/or leukocyte surface antigen cluster of differentiation CD47 (CD47).

### HLA-G/HLA-E Modifications

As used herein, the term “HLA-G” refers to the HLA non-classical class I heavy chain paralogues. This class I molecule is a heterodimer consisting of a heavy chain and a light chain (beta-2 microglobulin). The heavy chain is anchored in the membrane. HLA-G is expressed on fetal derived placental cells. HLA-G is a ligand for NK cell inhibitory receptor KIR2DL4, and therefore expression of this HLA by the trophoblast defends it against NK cell-mediated death. See e.g., Favier et al., Tolerogenic Function of Dimeric Forms of HLA-G Recombinant Proteins: A Comparative Study In Vivo PLOS One 2011, the entire contents of which are incorporated herein by reference. Exemplary sequences of HLA-G are provided in NG_029039.1 and set forth as SEQ ID NO: 1242.

HLA-G amino acid sequence
SEQ ID NO: 1242
MMVVMAPRILFLLLSGALTLTETWAGSHSMRYFSAAVSRPGRGEP
RFIAMGYVDDTQFVREDSDSACPRMEPRAPWVEQEGPEYWEEETR
NTKAHAQTDRMNLQTLRGYYNQSEASSHTLQWMIGCDLGSDGRLL
RGYEQYAYDGKDYLALNEDLRSWTAADTAAQISKRKCEAANVAEQ
RRAYLEGTCVEWLHRYLENGKEMLQRADPPKTHVTHHPVFDYEAT
LRCWALGFYPAEIILTWQRDGEDQTQDVELVETRPAGDGTFQKWA
AVVVPSGEEQRYTCHVQHEGLPEPLMLRWKQSSLPTIPIMGIVAG
LVVLAAVVTGAAVAAVLWRKKSSD

In some embodiments, an HLA-G nucleic acid sequence encoding a transgenic HLA-G gene may be fused to one or more non-HLA-G gene derived coding sequences. In some embodiments, an HLA-G nucleic acid coding sequence is fused directly or indirectly to a B2M gene derived nucleic acid coding sequence. In some embodiments, an HLA-G nucleic acid coding sequence is fused directly or indirectly to a peptide coding sequence. In some embodiments, an HLA-G nucleic acid coding sequence is fused directly or indirectly to a linker sequence. In some embodiments, an HLA-G nucleic acid coding sequence is comprised within a trimeric construct. In some embodiments, a trimeric HLA-G comprising construct comprises (in N to C terminal order) one or more N-terminal peptides, a linker sequence, a B2M gene derived sequence, a linker sequence, and an HLA-G sequence (see e.g., Gornalusse et al., Nature Biotech 2017). In some embodiments, a peptide encoding sequence, a B2M gene derived coding sequence, and/or an HLA-G coding sequence may be codon-optimized.

In some embodiments, a transgenic gene may additionally encode a linker sequence. Linker sequences are generally known in the art. Exemplary linker lengths are, e.g., between 1 and 200 amino acid residues, e.g., 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-110, 111-120, 121-130, 131-140, 141-150, 151-160, 161-170, 171-180, 181-190, or 191-200 amino acid residues. In some embodiments, a linker comprises about 1 to about 20 amino acid residues (e.g., about 5, 6, 7, 8, 9, 10, 11, 12, 13, 14, 15, 16, 17, 18, 19 or 20 amino acid residues). In some embodiments, a linker comprises about 5 to about 30 amino acids in length, e.g., between 10 and 20 amino acids in length, e.g., between 12 and 18 amino acids in length, e.g., 15 amino acids in length. In some embodiments, linkers can include or consist of flexible portions, e.g., regions without significant fixed secondary or tertiary structure. In some embodiments, a linker has an increased content of small amino acids, in particular of glycines, alanines, serines, threonines, leucines and/or isoleucines. For example, a linker may comprise at least 50%, at least 60%, at least 65%, at least 70%, at least 75%, at least 80%, at least 85%, at least 90%, at least 95%, or more glycine, serine, alanine, and/or threonine residues. Linkers may be glycine-rich linkers, e.g., comprising at least 50%, at least 60%, at least 65%, at least 70%, at least 75%, at least 80%, at least 85%, at least 90%, at least 95%, or more glycine residues. Linkers may be serine-rich linkers, e.g., comprising at least 50%, at least 60%, at least 65%, at least 70%, at least 75%, at least 80%, at least 85%, at least 90%, at least 95%, or more serine residues. In certain embodiments, a linker comprises at least 80%, at least 85%, at least 90%, at least 95%, or more glycine, serine, alanine, and/or threonine residues, and the remaining residues, if any, are glutamine, phenylalanine, and/lysine.

In some embodiments, a linker sequence comprises or consists of the amino acid sequence of SEQ ID NO: 1247 (or an amino acid sequence at least 90%, 95%, 98%, or more identical to SEQ ID NO: 1247). In some embodiments, a linker sequence comprises or consists of the amino acid sequence of SEQ ID NO: 1248 (or an amino acid sequence at least 90%, 95%, 98%, or more identical to SEQ ID NO: 1248).

Exemplary linker sequence
SEQ ID NO: 1247
GGGGSGGGGSGGGGS
Exemplary linker sequence
SEQ ID NO: 1248
GGGGSGGGGSGGGGSGGGGS

In some embodiments, a peptide-B2M-HLA-G transgene comprises or is SEQ ID NO: 1179. In some embodiments, a peptide-B2M-HLA-G transgene comprises a coding sequence that is 60%, 65%, 70%, 75%, 80%, 85%, 90%, 95%, or 99% identical to SEQ ID NO: 1179.

Trimeric peptide-B2M-HLA-G nucleic acid sequence
SEQ ID NO: 1179
ATGAGCCGGAGCGTGGCTCTGGCCGTGCTGGCCCTGCTGAGCCTG
AGCGGCCTCGAGGCTCGGATCATTCCTCGGCATCTGCAGCTGGGT
GGCGGTGGATCCGGTGGCGGTGGATCCGGTGGCGGTGGATCCATT
CAGCGGACCCCCAAAATCCAGGTGTACAGCCGGCACCCTGCTGAA
AACGGCAAAAGCAATTTTCTGAACTGCTATGTGAGCGGCTTCCAC
CCCAGCGATATCGAGGTGGACCTGCTGAAAAACGGCGAACGGATC
GAGAAAGTGGAACACAGCGACCTGAGCTTCAGCAAGGACTGGAGC
TTTTATCTGCTGTACTATACCGAGTTCACACCCACAGAGAAGGAT
GAGTATGCCTGCCGGGTGAACCACGTGACCCTGAGCCAGCCTAAA
ATCGTGAAGTGGGATCGGGATATGGGTGGCGGTGGATCCGGTGGC
GGTGGATCCGGTGGCGGTGGATCCGGTGGCGGTGGATCCGGCAGC
CATAGCATGCGGTATTTCAGCGCCGCTGTGAGCCGGCCTGGCCGG
GGCGAACCTCGGTTTATTGCCATGGGCTATGTGGACGATACCCAG
TTCGTGCGGTTTGATAGCGATAGCGCCTGTCCACGGATGGAGCCT
CGGGCCCCCTGGGTGGAGCAGGAAGGCCCCGAATATTGGGAAGAG
GAAACACGGAATACAAAGGCTCACGCCCAGACAGATCGGATGAAT
CTGCAGACACTGCGGGGCTACTATAACCAGAGCGAGGCTAGCAGC
CACACCCTGCAGTGGATGATTGGCTGTGACCTGGGCAGCGATGGC
CGGCTGCTGCGGGGCTACGAGCAGTACGCCTATGATGGCAAGGAC
TACCTGGCTCTGAACGAGGACCTGCGGAGCTGGACAGCCGCTGAC
ACCGCCGCTCAGATTAGCAAGCGGAAGTGTGAGGCTGCCAACGTG
GCTGAACAGCGGCGGGCTTATCTGGAGGGCACATGTGTGGAATGG
CTGCACCGGTACCTGGAGAATGGCAAAGAGATGCTGCAGCGGGCC
GACCCCCCAAAAACCCACGTGACCCACCATCCCGTGTTCGACTAC
GAGGCTACCCTGCGGTGTTGGGCCCTGGGCTTTTATCCTGCCGAG
ATCATTCTGACATGGCAGCGGGATGGCGAGGATCAGACACAGGAT
GTGGAGCTGGTGGAGACACGGCCAGCCGGCGATGGCACCTTTCAG
AAATGGGCCGCTGTGGTGGTGCCTAGCGGCGAAGAGCAGCGGTAC
ACATGCCATGTGCAGCATGAAGGCCTGCCAGAACCCCTGATGCTG
CGGTGGAAACAGAGCAGCCTGCCCACAATCCCTATCATGGGCATC
GTGGCTGGCCTGGTGGTGCTGGCCGCTGTGGTGACAGGCGCCGCT
GTGGCCGCTGTGCTGTGGCGGAAGAAAAGCAGCGAC

In some embodiments, a peptide-B2M-HLA-G transgenic amino acid sequence comprises or is SEQ ID NO: 1180. In some embodiments, a peptide-B2M-HLA-G amino acid sequence comprises a coding sequence that is 60%, 65%, 70%, 75%, 80%, 85%, 90%, 95%, or 99% identical to SEQ ID NO: 1180. In some embodiments, a transgenic amino acid sequence comprises or is a functional variant of SEQ ID NO: 1180. In some embodiments, a transgenic amino acid sequence comprises or is an amino acid sequence comprising 1, 2, 3, 4, 5, 6, 7, 8, 9, or 10 or more mutations (e.g., amino acid substitutions, insertions, and/or deletions) as compared to SEQ ID NO: 1180. In some embodiments, a peptide-B2M-HLA-G transgenic amino acid comprises or consists of an amino acid sequence of SEQ ID NO: 1180 lacking about 1 to about 25 amino acids at the N-terminus (e.g., lacking about 1-24, about 1-23, about 1-22, about 1-21, about 1-20, about 1-19, about 1-18, about 1-17, about 1-16, about 1-15, about 2-24, about 2-23, about 2-22, about 2-21, about 2-20, about 2-19, about 2-18, about 2-17, about 2-16, or about 2-15 of the amino acids at the N-terminus of SEQ ID NO: 1180).

Trimeric peptide-B2M-HLA-G amino acid
sequence (residues 21-29 correspond
to peptide, residues 1-20 and 45-143
correspond to B2M, residues 164-477
correspond to HLA-G)
SEQ ID NO: 1180
MSRSVALAVLALLSLSGLEARIIPRHLQLGGGGSGGGGSGGGGSI
QRTPKIQVYSRHPAENGKSNFLNCYVSGFHPSDIEVDLLKNGERI
EKVEHSDLSFSKDWSFYLLYYTEFTPTEKDEYACRVNHVILSQPK
IVKWDRDMGGGGSGGGGSGGGGSGGGGSGSHSMRYFSAAVSRPGR
GEPRFIAMGYVDDTQFVRFDSDSACPRMEPRAPWVEQEGPEYWEE
ETRNTKAHAQTDRMNLQTLRGYYNQSEASSHILQWMIGCDLGSDG
RLLRGYEQYAYDGKDYLALNEDLRSWTAADTAAQISKRKCEAANV
AEQRRAYLEGTCVEWLHRYLENGKEMLQRADPPKTHVTHHPVFDY
EATLRCWALGFYPAEIILTWQRDGEDQTQDVELVETRPAGDGTFQ
KWAAVVVPSGEEQRYTCHVQHEGLPEPLMLRWKQSSLPTIPIMGI
VAGLVVLAAVVIGAAVAAVLWRKKSSD

As used herein, the term “HLA-E” refers to the HLA class I histocompatibility antigen, alpha chain E, also sometimes referred to as MHC class I antigen E. The HLA-E protein in humans is encoded by the HLA-E gene. The human HLA-E is a non-classical MHC class I molecule that is characterized by a limited polymorphism and a lower cell surface expression than its classical paralogues. This class I molecule is a heterodimer consisting of a heavy chain and a light chain (beta-2 microglobulin). The heavy chain is anchored in the membrane. HLA-E binds a restricted subset of peptides derived from the leader peptides of other class I molecules. In some embodiments, HLA-E expressing cells may escape allogeneic responses and lysis by NK cells. See e.g., Geornalusse-G et al., Nature Biotechnology 2017 35(8), the entire contents of which are incorporated herein by reference. Exemplary sequences of the HLA-E protein are provided in NM_005516.6 and set forth as SEQ ID NO: 1240.

HLA-E amino acid sequence
SEQ ID NO: 1240
MVDGTLLLLLSEALALTQTWAGSHSLKYFHISVSRPGRGEPRFIS
VGYVDDTQFVREDNDAASPRMVPRAPWMEQEGSEYWDRETRSARD
TAQIFRVNLRTLRGYYNQSEAGSHTLQWMHGCELGPDGRFLRGYE
QFAYDGKDYLTLNEDLRSWTAVDTAAQISEQKSNDASEAEHQRAY
LEDTCVEWLHKYLEKGKETLLHLEPPKTHVTHHPISDHEATLRCW
ALGFYPAEITLTWQQDGEGHTQDTELVETRPAGDGTFQKWAAVVV
PSGEEQRYTCHVQHEGLPEPVILRWKPASQPTIPIVGIIAGLVLL
GSVVSGAVVAAVIWRKKSSGGKGGSYSKAEWSDSAQGSESHSL

In some embodiments, an HLA-E nucleic acid sequence encoding a transgenic HLA-E gene may be fused to one or more non-HLA-E gene derived coding sequences. In some embodiments, an HLA-E nucleic acid coding sequence is fused directly or indirectly to a B2M gene derived nucleic acid coding sequence. In some embodiments, an HLA-E nucleic acid coding sequence is fused directly or indirectly to a peptide (e.g., an HLA-G signal peptide) coding sequence. In some embodiments, an HLA-E nucleic acid coding sequence is fused directly or indirectly to a linker sequence. In some embodiments, an HLA-E nucleic acid coding sequence is comprised within a trimeric construct. In some embodiments, a trimeric HLA-E comprising construct comprises (in N to C terminal order) one or more N-terminal peptides (e.g., HLA-G signal peptides), a linker sequence, a B2M gene derived sequence, a linker sequence, and an HLA-E sequence (see e.g., Gornalusse et al., Nature Biotech 2017). In some embodiments, a peptide (e.g., an HLA-G signal peptide) encoding sequence, a B2M gene derived coding sequence, and/or an HLA-E coding sequence may be codon-optimized.

In some embodiments, an HLA-G signal peptide-B2M-HLA-E transgene comprises or is SEQ ID NO: 1181 or 1230. In some embodiments, an HLA-G signal peptide-B2M-HLA-E transgene comprises a coding sequence that is 60%, 65%, 70%, 75%, 80%, 85%, 90%, 95%, or 99% identical to SEQ ID NO: 1181 or 1230.

Trimeric HLA-G signal peptide-B2M-HLA-E
nucleic acid sequence
SEQ ID NO: 1181
ATGAGCCGGAGCGTGGCTCTGGCCGTGCTGGCCCTGCTGAGCCTG
AGCGGCCTCGAGGCTGTGATGGCCCCTCGGACCCTGATTCTGGGT
GGCGGTGGATCCGGTGGCGGTGGATCCGGTGGCGGTGGATCCATT
CAGCGGACACCCAAAATCCAGGTGTACAGCCGGCATCCCGCCGAA
AACGGCAAGAGCAATTTCCTGAACTGTTACGTGAGCGGCTTCCAC
CCCAGCGACATTGAAGTGGACCTGCTGAAAAACGGCGAGCGGATT
GAAAAAGTGGAACACAGCGACCTGAGCTTTAGCAAAGATTGGAGC
TTTTACCTGCTGTATTACACCGAATTCACCCCCACCGAGAAGGAT
GAGTACGCCTGCCGGGTGAACCATGTGACCCTGAGCCAGCCAAAA
ATCGTGAAGTGGGATCGGGATATGGGTGGCGGTGGATCCGGTGGC
GGTGGATCCGGTGGCGGTGGATCCGGTGGCGGTGGATCCGGCAGC
CATAGCCTGAAATACTTTCACACCAGCGTGAGCCGGCCTGGCCGG
GGCGAGCCACGGTTTATCAGCGTGGGCTATGTGGACGATACCCAG
TTTGTGCGGTTTGACAATGACGCTGCCAGCCCTCGGATGGTGCCA
CGGGCTCCCTGGATGGAACAGGAGGGCAGCGAATATTGGGACCGG
GAAACCCGGAGCGCCCGGGATACCGCCCAGATTTTCCGGGTGAAT
CTGCGGACCCTGCGGGGCTACTATAACCAGAGCGAAGCTGGCAGC
CATACACTGCAGTGGATGCACGGCTGTGAGCTGGGCCCAGATGGC
CGGTTCCTGCGGGGCTATGAACAGTTTGCCTATGATGGCAAAGAC
TATCTGACACTGAATGAAGACCTGCGGAGCTGGACCGCCGTGGAC
ACAGCTGCCCAGATTAGCGAGCAGAAGAGCAATGATGCCAGCGAG
GCCGAGCATCAGCGGGCTTACCTGGAGGACACATGCGTGGAGTGG
CTGCATAAATATCTGGAAAAAGGCAAGGAGACACTGCTGCATCTG
GAACCTCCAAAGACCCACGTGACACACCATCCTATTAGCGATCAC
GAGGCTACCCTGCGGTGCTGGGCCCTGGGCTTCTACCCCGCCGAG
ATCACCCTGACCTGGCAGCAGGATGGCGAAGGCCACACCCAGGAT
ACCGAGCTGGTGGAAACACGGCCTGCCGGCGACGGCACATTCCAG
AAGTGGGCTGCCGTGGTGGTGCCCAGCGGCGAAGAGCAGCGGTAC
ACCTGCCATGTGCAGCACGAAGGCCTGCCTGAACCAGTGACCCTG
CGGTGGAAACCAGCCAGCCAGCCCACCATCCCCATCGTGGGCATT
ATCGCTGGCCTGGTGCTGCTGGGCAGCGTGGTGAGCGGCGCCGTG
GTGGCCGCTGTGATTTGGCGGAAGAAAAGCAGCGGCGGCAAAGGC
GGCAGCTACAGCAAGGCCGAGTGGAGCGACAGCGCTCAGGGCAGC
GAAAGCCACAGCCTG
Trimeric HLA-G signal peptide-B2M-HLA-E
nucleic acid sequence
SEQ ID NO: 1230
ATGAGCCGGAGCGTGGCTCTGGCCGTGCTGGCCCTGCTGAGCCTG
AGCGGCCTCGAGGCTGTGATGGCCCCTCGGACCCTGATTCTGGGT
GGCGGTGGATCCGGTGGCGGTGGATCCGGTGGCGGTGGATCCATT
CAGCGGACACCCAAAATCCAGGTGTACAGCCGGCATCCCGCCGAA
AACGGCAAGAGCAATTTCCTGAACTGTTACGTGAGCGGCTTCCAC
CCCAGCGACATTGAAGTGGACCTGCTGAAAAACGGCGAGCGGATT
GAAAAAGTGGAACACAGCGACCTGAGCTTTAGCAAAGATTGGAGC
TTTTACCTGCTGTATTACACCGAATTCACCCCCACCGAGAAGGAT
GAGTACGCCTGCCGGGTGAACCATGTGACCCTGAGCCAGCCAAAA
ATCGTGAAGTGGGATCGGGATATGGGTGGCGGTGGATCCGGTGGC
GGTGGATCCGGTGGCGGTGGATCCGGCAGCCATAGCCTGAAATAC
TTTCACACCAGCGTGAGCCGGCCTGGCCGGGGCGAGCCACGGTTT
ATCAGCGTGGGCTATGTGGACGATACCCAGTTTGTGCGGTTTGAC
AATGACGCTGCCAGCCCTCGGATGGTGCCACGGGCTCCCTGGATG
GAACAGGAGGGCAGCGAATATTGGGACCGGGAAACCCGGAGCGCC
CGGGATACCGCCCAGATTTTCCGGGTGAATCTGCGGACCCTGCGG
GGCTACTATAACCAGAGCGAAGCTGGCAGCCATACACTGCAGTGG
ATGCACGGCTGTGAGCTGGGCCCAGATGGCCGGTTCCTGCGGGGC
TATGAACAGTTTGCCTATGATGGCAAAGACTATCTGACACTGAAT
GAAGACCTGCGGAGCTGGACCGCCGTGGACACAGCTGCCCAGATT
AGCGAGCAGAAGAGCAATGATGCCAGCGAGGCCGAGCATCAGCGG
GCTTACCTGGAGGACACATGCGTGGAGTGGCTGCATAAATATCTG
GAAAAAGGCAAGGAGACACTGCTGCATCTGGAACCTCCAAAGACC
CACGTGACACACCATCCTATTAGCGATCACGAGGCTACCCTGCGG
TGCTGGGCCCTGGGCTTCTACCCCGCCGAGATCACCCTGACCTGG
CAGCAGGATGGCGAAGGCCACACCCAGGATACCGAGCTGGTGGAA
ACACGGCCTGCCGGCGACGGCACATTCCAGAAGTGGGCTGCCGTG
GTGGTGCCCAGCGGCGAAGAGCAGCGGTACACCTGCCATGTGCAG
CACGAAGGCCTGCCTGAACCAGTGACCCTGCGGTGGAAACCAGCC
AGCCAGCCCACCATCCCCATCGTGGGCATTATCGCTGGCCTGGTG
CTGCTGGGCAGCGTGGTGAGCGGCGCCGTGGTGGCCGCTGTGATT
TGGCGGAAGAAAAGCAGCGGCGGCAAAGGCGGCAGCTACAGCAAG
GCCGAGTGGAGCGACAGCGCTCAGGGCAGCGAAAGCCACAGCCTG

In some embodiments, an HLA-G signal peptide-B2M-HLA-E transgenic amino acid sequence comprises or is SEQ ID NO: 1182, 1231, 1243, 1244, or 1245. In some embodiments, an HLA-G signal peptide-B2M-HLA-E amino acid sequence comprises a coding sequence that is 60%, 65%, 70%, 75%, 80%, 85%, 90%, 95%, or 99% identical to SEQ ID NO: 1182, 1231, 1243, 1244, or 1245. In some embodiments, a transgenic amino acid sequence comprises or is a functional variant of SEQ ID NO: 1182, 1231, 1243, 1244, or 1245. In some embodiments, a transgenic amino acid sequence comprises or is an amino acid sequence comprising 1, 2, 3, 4, 5, 6, 7, 8, 9, or 10 or more mutations (e.g., substitutions, insertions, and/or deletions) as compared to SEQ ID NO: 1182, 1231, 1243, 1244, or 1245. In some embodiments, an HLA-G signal peptide-B2M-HLA-E transgenic amino acid comprises or consists of an amino acid sequence of SEQ ID NO: 1182, 1231, 1243, 1244, or 1245, and lacking about 1 to about 25 amino acids at the N-terminus (e.g., lacking about 1-24, about 1-23, about 1-22, about 1-21, about 1-20, about 1-19, about 1-18, about 1-17, about 1-16, about 1-15, about 2-24, about 2-23, about 2-22, about 2-21, about 2-20, about 2-19, about 2-18, about 2-17, about 2-16, or about 2-15 of the amino acids at the N-terminus of SEQ ID NO: 1182, 1231, 1243, 1244, or 1245).

In some embodiments, an HLA-E transgenic amino acid sequence comprises or is SEQ ID NO: 1246. In some embodiments, an HLA-E transgenic amino acid sequence amino acid sequence comprises a coding sequence that is 60%, 65%, 70%, 75%, 80%, 85%, 90%, 95%, or 99% identical to SEQ ID NO: 1246. In some embodiments, a transgenic amino acid sequence comprises or is a functional variant of SEQ ID NO: 1246. In some embodiments, a transgenic amino acid sequence comprises or is an amino acid sequence comprising 1, 2, 3, 4, 5, 6, 7, 8, 9, or 10 or more mutations (e.g., substitutions, insertions, and/or deletions) as compared to SEQ ID NO: 1246. In some embodiments, a transgenic amino acid comprises or consists of an amino acid sequence of SEQ ID NO: 1246, and lacking about 1 to about 25 amino acids at the N-terminus (e.g., lacking about 1-24, about 1-23, about 1-22, about 1-21, about 1-20, about 1-19, about 1-18, about 1-17, about 1-16, about 1-15, about 2-24, about 2-23, about 2-22, about 2-21, about 2-20, about 2-19, about 2-18, about 2-17, about 2-16, or about 2-15 of the amino acids at the N-terminus of SEQ ID NO: 1246).

Trimeric HLA-G signal peptide-B2M-HLA-E
amino acid sequence (residues 21-29 correspond
to HLA-G signal peptide, residues 1-20 and
45-143 correspond to B2M, residues 164-500
correspond to HLA-E)
SEQ ID NO: 1182
MSRSVALAVLALLSLSGLEAVMAPRTLILGGGGSGGGGSGGGGSI
QRTPKIQVYSRHPAENGKSNFLNCYVSGFHPSDIEVDLLKNGERI
EKVEHSDLSFSKDWSFYLLYYTEFTPTEKDEYACRVNHVTLSQPK
IVKWDRDMGGGGSGGGGSGGGGSGGGGSGSHSLKYFHTSVSRPGR
GEPRFISVGYVDDTQFVRFDNDAASPRMVPRAPWMEQEGSEYWDR
ETRSARDTAQIFRVNLRTLRGYYNQSEAGSHILQWMHGCELGPDG
RFLRGYEQFAYDGKDYLILNEDLRSWTAVDTAAQISEQKSNDASE
AEHQRAYLEDTCVEWLHKYLEKGKETLLHLEPPKTHVTHHPISDH
EATLRCWALGFYPAEITLTWQQDGEGHTQDTELVETRPAGDGTFQ
KWAAVVVPSGEEQRYTCHVQHEGLPEPVTLRWKPASQPTIPIVGI
IAGLVLLGSVVSGAVVAAVIWRKKSSGGKGGSYSKAEWSDSAQGS
ESHSL
Trimeric HLA-G signal peptide-B2M-HLA-E
amino acid sequence (residues 21-29 correspond
to HLA-G signal peptide, residues 1-20 and
45-143 correspond to B2M, residues 159-495
correspond to HLA-E)
SEQ ID NO: 1231
MSRSVALAVLALLSLSGLEAVMAPRILILGGGGSGGGGSGGGGSI
QRTPKIQVYSRHPAENGKSNFLNCYVSGFHPSDIEVDLLKNGERI
EKVEHSDLSFSKDWSFYLLYYTEFTPTEKDEYACRVNHVTLSQPK
IVKWDRDMGGGGSGGGGSGGGGSGSHSLKYFHTSVSRPGRGEPRF
ISVGYVDDTQFVRFDNDAASPRMVPRAPWMEQEGSEYWDRETRSA
RDTAQIFRVNLRTLRGYYNQSEAGSHTLQWMHGCELGPDGRFLRG
YEQFAYDGKDYLTLNEDLRSWTAVDTAAQISEQKSNDASEAEHQR
AYLEDTCVEWLHKYLEKGKETLLHLEPPKTHVTHHPISDHEATLR
CWALGFYPAEITLTWQQDGEGHTQDTELVETRPAGDGTFQKWAAV
VVPSGEEQRYTCHVQHEGLPEPVTLRWKPASQPTIPIVGIIAGLV
LLGSVVSGAVVAAVIWRKKSSGGKGGSYSKAEWSDSAQGSESHSL
Trimeric HLA-G signal peptide-B2M-HLA-E
amino acid sequence(residues 21-29
correspond to HLA-G signal peptide,
residues 1-20 and 45-143 correspond to
B2M, residues 164-500 correspond to HLA-E)
SEQ ID NO: 1243
MSRSVALAVLALLSLSGLEAVMAPRILVLGGGGSGGGGSGGGGSI
QRTPKIQVYSRHPAENGKSNFLNCYVSGFHPSDIEVDLLKNGERI
EKVEHSDLSFSKDWSFYLLYYTEFTPTEKDEYACRVNHVTLSQPK
IVKWDRDMGGGGSGGGGSGGGGSGGGGSGSHSLKYFHTSVSRPGR
GEPRFISVGYVDDTQFVRFDNDAASPRMVPRAPWMEQEGSEYWDR
ETRSARDTAQIFRVNLRTLRGYYNQSEAGSHTLQWMHGCELGPDG
RFLRGYEQFAYDGKDYLILNEDLRSWTAVDTAAQISEQKSNDASE
AEHQRAYLEDTCVEWLHKYLEKGKETLLHLEPPKTHVTHHPISDH
EATLRCWALGFYPAEITLTWQQDGEGHTQDTELVETRPAGDGTFQ
KWAAVVVPSGEEQRYTCHVQHEGLPEPVTLRWKPASQPTIPIVGI
IAGLVLLGSVVSGAVVAAVIWRKKSSGGKGGSYSKAEWSDSAQGS
ESHSL
Trimeric HLA-G signal peptide-B2M-HLA-E
amino acid sequence (residues 21-29 correspond
to HLA-G signal peptide, residues 1-20 and
45-143 correspond to B2M, residues 164-500
correspond to HLA-E)
SEQ ID NO: 1244
MSRSVALAVLALLSLSGLEAVMAPRILFLGGGGSGGGGSGGGGSI
QRTPKIQVYSRHPAENGKSNFLNCYVSGFHPSDIEVDLLKNGERI
EKVEHSDLSFSKDWSFYLLYYTEFTPTEKDEYACRVNHVTLSQPK
IVKWDRDMGGGGSGGGGSGGGGSGGGGSGSHSLKYFHTSVSRPGR
GEPRFISVGYVDDTQFVRFDNDAASPRMVPRAPWMEQEGSEYWDR
ETRSARDTAQIFRVNLRTLRGYYNQSEAGSHTLQWMHGCELGPDG
RFLRGYEQFAYDGKDYLTLNEDLRSWTAVDTAAQISEQKSNDASE
AEHQRAYLEDTCVEWLHKYLEKGKETLLHLEPPKTHVTHHPISDH
EATLRCWALGFYPAEITLTWQQDGEGHTQDTELVETRPAGDGTFQ
KWAAVVVPSGEEQRYTCHVQHEGLPEPVTLRWKPASQPTIPIVGI
IAGLVLLGSVVSGAVVAAVIWRKKSSGGKGGSYSKAEWSDSAQGS
ESHSL
Trimeric HLA-G signal peptide-B2M-HLA-E
amino acid sequence (residues 21-29 correspond
to HLA-G signal peptide, residues 1-20 and
45-143 correspond to B2M, residues
164-500 correspond to HLA-E)
SEQ ID NO: 1245
MSRSVALAVLALLSLSGLEAVMAPRTVLLGGGGSGGGGSGGGGSI
QRTPKIQVYSRHPAENGKSNFLNCYVSGFHPSDIEVDLLKNGERI
EKVEHSDLSFSKDWSFYLLYYTEFTPTEKDEYACRVNHVTLSQPK
IVKWDRDMGGGGSGGGGSGGGGSGGGGSGSHSLKYFHTSVSRPGR
GEPRFISVGYVDDTQFVRFDNDAASPRMVPRAPWMEQEGSEYWDR
ETRSARDTAQIFRVNLRTLRGYYNQSEAGSHTLQWMHGCELGPDG
RFLRGYEQFAYDGKDYLTLNEDLRSWTAVDTAAQISEQKSNDASE
AEHQRAYLEDTCVEWLHKYLEKGKETLLHLEPPKTHVTHHPISDH
EATLRCWALGFYPAEITLTWQQDGEGHTQDTELVETRPAGDGTFQ
KWAAVVVPSGEEQRYTCHVQHEGLPEPVTLRWKPASQPTIPIVGI
IAGLVLLGSVVSGAVVAAVIWRKKSSGGKGGSYSKAEWSDSAQGS
ESHSL
Trimeric peptide-B2M-HLA-E amino acid
sequence (residues 21-29
correspond to peptide, residues 1-20
and 45-143 correspond to B2M, residues
164-500 correspond to HLA-E)
SEQ ID NO: 1246
MSRSVALAVLALLSLSGLEARIIPRHLQLGGGGSGGGGSGGGGSI
QRTPKIQVYSRHPAENGKSNFLNCYVSGFHPSDIEVDLLKNGERI
EKVEHSDLSFSKDWSFYLLYYTEFTPTEKDEYACRVNHVTLSQPK
IVKWDRDMGGGGSGGGGSGGGGSGGGGSGSHSLKYFHTSVSRPGR
GEPRFISVGYVDDTQFVRFDNDAASPRMVPRAPWMEQEGSEYWDR
ETRSARDTAQIFRVNLRTLRGYYNQSEAGSHTLQWMHGCELGPDG
RFLRGYEQFAYDGKDYLILNEDLRSWTAVDTAAQISEQKSNDASE
AEHQRAYLEDTCVEWLHKYLEKGKETLLHLEPPKTHVTHHPISDH
EATLRCWALGFYPAEITLTWQQDGEGHTQDTELVETRPAGDGTFQ
KWAAVVVPSGEEQRYTCHVQHEGLPEPVTLRWKPASQPTIPIVGI
IAGLVLLGSVVSGAVVAAVIWRKKSSGGKGGSYSKAEWSDSAQGS
ESHSL

In some embodiments, an HLA-E transgene encodes an HLA-E polypeptide (e.g., an amino acid sequence having about 80%, 85%, 90%, 91%, 92%, 93%, 94%, 95%, 96%, 97%, 98%, 99%, or 100% identity to SEQ ID NO: 1251; or an amino acid sequence having 80%, 85%, 90%, 91%, 92%, 93%, 94%, 95%, 96%, 97%, 98%, 99%, or 100% identity to a portion of SEQ ID NO: 1251 (e.g., lacking 1, 2, 3, 4, or 5 amino acid residues from the N and/or C terminus of SEQ ID NO: 1251)).

In some embodiments, an HLA-E transgene encodes a B2M polypeptide (e.g., an amino acid sequence having about 80%, 85%, 90%, 91%, 92%, 93%, 94%, 95%, 96%, 97%, 98%, 99%, or 100% identity to SEQ ID NO: 1250; or an amino acid sequence having 80%, 85%, 90%, 91%, 92%, 93%, 94%, 95%, 96%, 97%, 98%, 99%, or 100% identity to a portion of SEQ ID NO: 1250 (e.g., lacking 1, 2, 3, 4, or 5 amino acid residues from the N and/or C terminus of SEQ ID NO: 1250)).

In some embodiments, an HLA-E transgene encodes a peptide, e.g., an HLA-G signal peptide. In some embodiments, an HLA-E transgene encodes a peptide, e.g., a peptide comprising an amino acid sequence having 80%, 85%, 90%, 91%, 92%, 93%, 94%, 95%, 96%, 97%, 98%, 99%, or 100% identity to RIIPRHLQL (SEQ ID NO: 1234), VMAPRTLFL (SEQ ID NO: 1235), VMAPRTLIL (SEQ ID NO: 1236), VMAPRTVLL (SEQ ID NO: 1237), and/or VMAPRTLVL (SEQ ID NO: 1238)).

In some embodiments, an HLA-E transgene encodes (i) a B2M polypeptide (e.g., an amino acid sequence having about 80%, 85%, 90%, 91%, 92%, 93%, 94%, 95%, 96%, 97%, 98%, 99%, or 100% identity to SEQ ID NO: 1250; or an amino acid sequence having 80%, 85%, 90%, 91%, 92%, 93%, 94%, 95%, 96%, 97%, 98%, 99%, or 100% identity to a portion of SEQ ID NO: 1250 (e.g., lacking 1, 2, 3, 4, or 5 amino acid residues from the N and/or C terminus of SEQ ID NO: 1250)); and (ii) an HLA-E polypeptide (e.g., an amino acid sequence having about 80%, 85%, 90%, 91%, 92%, 93%, 94%, 95%, 96%, 97%, 98%, 99%, or 100% identity to SEQ ID NO: 1251; or an amino acid sequence having 80%, 85%, 90%, 91%, 92%, 93%, 94%, 95%, 96%, 97%, 98%, 99%, or 100% identity to a portion of SEQ ID NO: 1251 (e.g., lacking 1, 2, 3, 4, or 5 amino acid residues from the N and/or C terminus of SEQ ID NO: 1251)).

In some embodiments, an HLA-E transgene encodes (i) a peptide, e.g., an HLA-G signal peptide (e.g., an amino acid sequence having 80%, 85%, 90%, 91%, 92%, 93%, 94%, 95%, 96%, 97%, 98%, 99%, or 100% identity to VMAPRTLFL (SEQ ID NO: 1235), VMAPRTLIL (SEQ ID NO: 1236), VMAPRTVLL (SEQ ID NO: 1237), and/or VMAPRTLVL (SEQ ID NO: 1238)); (ii) a B2M polypeptide (e.g., an amino acid sequence having about 80%, 85%, 90%, 91%, 92%, 93%, 94%, 95%, 96%, 97%, 98%, 99%, or 100% identity to SEQ ID NO: 1250; or an amino acid sequence having 80%, 85%, 90%, 91%, 92%, 93%, 94%, 95%, 96%, 97%, 98%, 99%, or 100% identity to a portion of SEQ ID NO: 1250 (e.g., lacking 1, 2, 3, 4, or 5 amino acid residues from the N and/or C terminus of SEQ ID NO: 1250)); and (iii) an HLA-E polypeptide (e.g., an amino acid sequence having about 80%, 85%, 90%, 91%, 92%, 93%, 94%, 95%, 96%, 97%, 98%, 99%, or 100% identity to SEQ ID NO: 1251; or an amino acid sequence having 80%, 85%, 90%, 91%, 92%, 93%, 94%, 95%. 96%, 97%, 98%, 99%, or 100% identity to a portion of SEQ ID NO: 1251 (e.g., lacking 1, 2, 3, 4, or 5 amino acid residues from the N and/or C terminus of SEQ ID NO: 1251)). In some embodiments, an HLA-E transgene encodes (i) a peptide comprising an amino acid sequence having 80%, 85%, 90%, 91%, 92%, 93%, 94%, 95%, 96%, 97%, 98%, 99%, or 100% identity to SEQ ID NO:1234; (ii) a B2M polypeptide (e.g., an amino acid sequence having about 80%, 85%, 90%, 91%, 92%, 93%, 94%, 95%, 96%, 97%, 98%, 99%, or 100% identity to SEQ ID NO: 1250; or an amino acid sequence having 80%, 85%, 90%, 91%, 92%, 93%. 94%, 95%, 96%, 97%, 98%, 99%, or 100% identity to a portion of SEQ ID NO: 1250 (e.g., lacking 1, 2, 3, 4, or 5 amino acid residues from the N and/or C terminus of SEQ ID NO: 1250)); and (iii) an HLA-E polypeptide (e.g., an amino acid sequence having about 80%, 85%, 90%, 91%, 92%, 93%, 94%, 95%, 96%, 97%, 98%, 99%, or 100% identity to SEQ ID NO: 1251; or an amino acid sequence having 80%, 85%, 90%, 91%, 92%, 93%, 94%, 95%. 96%, 97%, 98%, 99%, or 100% identity to a portion of SEQ ID NO: 1251 (e.g., lacking 1, 2, 3, 4, or 5 amino acid residues from the N and/or C terminus of SEQ ID NO: 1251)).

In some embodiments, an HLA-E transgene encodes (i) a signal sequence (e.g., an amino acid sequence having at least 80%, 85%, 90%, 91%, 92%, 93%, 94%, 95%, 96%, 97%, 98%, 99%, or 100% identity to SEQ ID NO: 1249; or an amino acid sequence having 80%, 85%, 90%, 91%, 92%, 93%, 94%, 95%, 96%, 97%, 98%, 99%, or 100% identity to a portion of SEQ ID NO: 1249 (e.g., lacking 1, 2, 3, 4, or 5 amino acid residues from the N and/or C terminus of SEQ ID NO: 1249)); (ii) an HLA-G signal peptide (e.g., an amino acid sequence having 80%, 85%, 90%, 91%, 92%, 93%, 94%, 95%, 96%, 97%, 98%, 99%, or 100% identity to SEQ ID NO: 1235, 1236, 1237, or 1238); (iii) a B2M polypeptide (e.g., an amino acid sequence having about 80%, 85%, 90%, 91%, 92%, 93%, 94%, 95%, 96%, 97%, 98%, 99%, or 100% identity to SEQ ID NO: 1250; or an amino acid sequence having 80%, 85%, 90%, 91%, 92%, 93%, 94%, 95%, 96%, 97%, 98%, 99%, or 100% identity to a portion of SEQ ID NO: 1250 (e.g., lacking 1, 2, 3, 4, or 5 amino acid residues from the N and/or C terminus of SEQ ID NO: 1250)); and (iv) an HLA-E polypeptide (e.g., an amino acid sequence having about 80%, 85%, 90%, 91%, 92%, 93%, 94%, 95%, 96%, 97%, 98%, 99%, or 100% identity to SEQ ID NO: 1251; or an amino acid sequence having 80%, 85%, 90%, 91%, 92%, 93%, 94%, 95%, 96%, 97%, 98%, 99%, or 100% identity to a portion of SEQ ID NO: 1251 (e.g., lacking 1, 2, 3, 4, or 5 amino acid residues from the N and/or C terminus of SEQ ID NO: 1251)). In some embodiments, an HLA-E transgene encodes (i) a signal sequence (e.g., an amino acid sequence having at least 80%, 85%, 90%, 91%, 92%, 93%, 94%, 95%, 96%, 97%, 98%, 99%, or 100% identity to SEQ ID NO: 1249; or an amino acid sequence having 80%, 85%, 90%, 91%, 92%, 93%, 94%, 95%, 96%, 97%, 98%, 99%, or 100% identity to a portion of SEQ ID NO: 1249 (e.g., lacking 1, 2, 3, 4, or 5 amino acid residues from the N and/or C terminus of SEQ ID NO: 1249)); (ii) a peptide comprising an amino acid sequence having 80%, 85%, 90%, 91%, 92%, 93%, 94%, 95%, 96%, 97%, 98%, 99%, or 100% identity to SEQ ID NO: 1234; (iii) a B2M polypeptide (e.g., an amino acid sequence having about 80%, 85%, 90%, 91%, 92%, 93%, 94%, 95%, 96%, 97%, 98%, 99%, or 100% identity to SEQ ID NO: 1250; or an amino acid sequence having 80%, 85%, 90%, 91%, 92%, 93%, 94%, 95%, 96%, 97%, 98%, 99%, or 100% identity to a portion of SEQ ID NO: 1250 (e.g., lacking 1, 2, 3, 4, or 5 amino acid residues from the N and/or C terminus of SEQ ID NO: 1250)); and (iv) an HLA-E polypeptide (e.g., an amino acid sequence having about 80%, 85%, 90%, 91%, 92%, 93%, 94%, 95%, 96%, 97%, 98%, 99%, or 100% identity to SEQ ID NO: 1251; or an amino acid sequence having 80%, 85%, 90%, 91%, 92%, 93%, 94%, 95%, 96%, 97%, 98%, 99%, or 100% identity to a portion of SEQ ID NO: 1251 (e.g., lacking 1, 2, 3, 4, or 5 amino acid residues from the N and/or C terminus of SEQ ID NO: 1251)).

Signal sequence
SEQ ID NO: 1249
MSRSVALAVLALLSLSGLEA
B2M polypeptide
SEQ ID NO: 1250
IQRTPKIQVYSRHPAENGKSNFLNCYVSGFHPSDIEVDLLKNGER
IEKVEHSDLSFSKDWSFYLLYYTEFTPTEKDEYACRVNHVILSQP
KIVKWDRDM
HLA-E polypeptide
SEQ ID NO: 1251
GSHSLKYFHTSVSRPGRGEPRFISVGYVDDTQFVRFDNDAASPRM
VPRAPWMEQEGSEYWDRETRSARDTAQIFRVNLRTLRGYYNQSEA
GSHTLQWMHGCELGPDGRFLRGYEQFAYDGKDYLTLNEDLRSWTA
VDTAAQISEQKSNDASEAEHQRAYLEDTCVEWLHKYLEKGKETLL
HLEPPKTHVTHHPISDHEATLRCWALGFYPAEITLTWQQDGEGHT
QDTELVETRPAGDGTFQKWAAVVVPSGEEQRYTCHVQHEGLPEPV
TLRWKPASQPTIPIVGIIAGLVLLGSVVSGAVVAAVIWRKKSSGG
KGGSYSKAEWSDSAQGSESHSL
HLA-G polypeptide
SEQ ID NO: 1252
GSHSMRYFSAAVSRPGRGEPRFIAMGYVDDTQFVREDSDSACPRM
EPRAPWVEQEGPEYWEEETRNTKAHAQTDRMNLQTLRGYYNQSEA
SSHTLQWMIGCDLGSDGRLLRGYEQYAYDGKDYLALNEDLRSWTA
ADTAAQISKRKCEAANVAEQRRAYLEGTCVEWLHRYLENGKEMLQ
RADPPKTHVTHHPVFDYEATLRCWALGFYPAEIILTWQRDGEDQT
QDVELVETRPAGDGTFQKWAAVVVPSGEEQRYTCHVQHEGLPEPL
MLRWKQSSLPTIPIMGIVAGLVVLAAVVIGAAVAAVLWRKKSSD

### Additional Gain-of-Function Modifications

In some embodiments, a genetically engineered stem cell and/or progeny cell, additionally or alternatively, comprises a genetic modification that leads to expression one or more of a CAR; a non-naturally occurring variant of FcγRIII (CD16); interleukin 15 (IL-15); an IL-15 receptor (IL-15R) agonist, or a constitutively active variant of an IL-15 receptor; interleukin 12 (IL-12); an IL-12 receptor (IL-12R) agonist, or a constitutively active variant of an IL-12 receptor; and/or leukocyte surface antigen cluster of differentiation CD47 (CD47).

As used herein, the term “chimeric antigen receptor” or “CAR” refers to a receptor protein that has been modified to give cells expressing the CAR the new ability to target a specific protein. Within the context of the disclosure, a cell modified to comprise a CAR may be used for immunotherapy to target and destroy cells associated with a disease or disorder, e.g., cancer cells. In some embodiments, the CAR can bind to any antigen of interest.

CARs of interest include, but are not limited to, a CAR targeting mesothelin, EGFR, HER2 and/or MICA/B. To date, mesothelin-targeted CAR T-cell therapy has shown early evidence of efficacy in a phase I clinical trial of subjects having mesothelioma, non-small cell lung cancer, and breast cancer (NCT02414269). Similarly, CARs targeting EGFR, HER2 and MICA/B have shown promise in early studies (see, e.g., Li et al. (2018), Cell Death & Disease, 9(177); Han et al. (2018) Am. J. Cancer Res., 8(1):106-119; and Demoulin (2017) Future Oncology, 13(8); the entire contents of each of which are expressly incorporated herein by reference in their entireties).

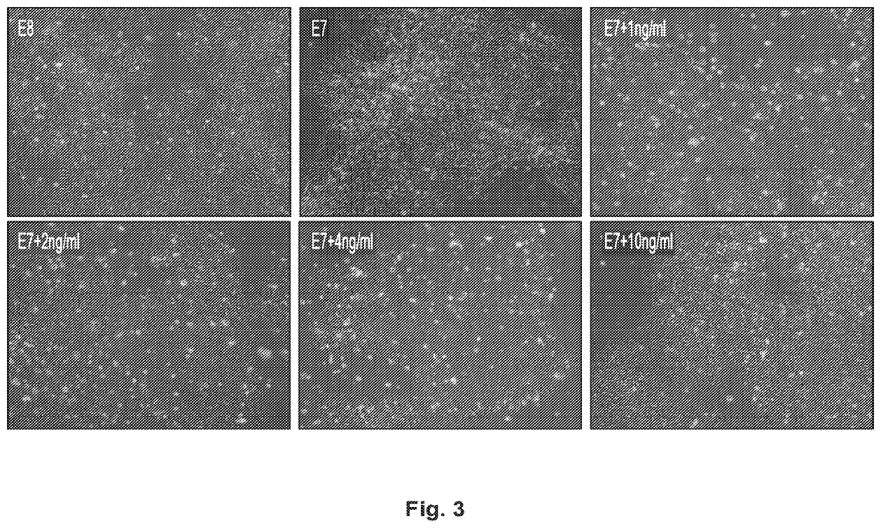

CARs are well-known to those of ordinary skill in the art and include those described in, for example: WO13/063419 (mesothelin), WO15/164594 (EGFR), WO13/063419 (HER2), and WO16/154585 (MICA and MICB), the entire contents of each of which are expressly incorporated herein by reference in their entireties. Any suitable CAR, NK-CAR, or other binder that targets a cell, e.g., an NK cell, to a target cell, e.g., a cell associated with a disease or disorder, may be expressed in the modified NK cells provided herein. Exemplary CARs, and binders, include, but are not limited to, bi-specific antigen binding CARs, switchable CARs, dimerizable CARs, split CARs, multi-chain CARs, inducible CARs, CARs and binders that bind BCMA, CD19, CD22, CD20, CD33, CD123, androgen receptor, PSMA, PSCA, Muc1, HPV viral peptides (e.g., E7), EBV viral peptides, CD70, WT1, CEA, EGFR, EGFRvIII, IL13Rα2, GD2, CA125, CD7, EpCAM, Muc16, carbonic anhydrase IX (CAIX), CCR1, CCR4, carcinoembryonic antigen (CEA), CD3, CD5, CD10, CD23, CD24, CD26, CD30, CD34, CD35, CD38, CD41, CD44, CD44V6, CD49f, CD56, CD92, CD99, CD133, CD135, CD148, CD150, CD261, CD362, CLEC12A, MDM2, CYP1B, livin, cyclin 1, NKp30, NKp46, DNAM1, NKp44, CA9, PD1, PDL1, an antigen of cytomegalovirus (CMV), epithelial glycoprotein-40 (EGP-40), GPRC5D, receptor tyrosine kinases erb-B2,3,4, EGFIR, ERBB folate binding protein (FBP), fetal acetylcholine receptor (AChR), folate receptor-a, ganglioside G3 (GD3) human Epidermal Growth Factor Receptor 2 (HER-2), human telomerase reverse transcriptase (hTERT), ICAM-1, Integrin B7, Interleukin-13 receptor subunit alpha-2 (IL-13Ra2), K-light chain, kinase insert domain receptor (KDR), Lewis A (CA19.9), Lewis Y (Le Y), L1 cell adhesion molecule (LI-CAM), LILRB2, melanoma antigen family A 1 (MAGE-A1), MICA/B, NKCSI, NKG2D ligands, c-Met, cancer-testis antigen NYESO-1, oncofetal antigen (h5T4), PRAME, tumor-associated glycoprotein 72 (TAG-72), TIM-3, TRBCI, TRBC2, vascular endothelial growth factor R2 (VEGF-R2), Wilms tumor protein (WT-1), a pathogen antigen, or any suitable combination thereof. Additional suitable CARs and binders for use in the modified NK cells provided herein will be apparent to those of skill in the art based on the present disclosure and the general knowledge in the art. Such additional suitable CARs include those described in FIG. 3 of Davies and Maher, Adoptive T-cell Immunotherapy of Cancer Using Chimeric Antigen Receptor-Grafted T Cells, Archivum Immunologiae et Therapiae Experimentalis 58(3): 165-78 (2010), the entire contents of which are incorporated herein by reference. Additional CARs suitable for methods described herein include: CD171-specific CARs (Park et al., Mol Ther (2007) 15(4):825-833), EGFRvIII-specific CARs (Morgan et al, Hum Gene Ther (2012) 23(10): 1043-1053), EGF-R-specific CARs (Kobold et al, J Natl Cancer Inst (2014) 107(1):364), carbonic anhydrase K-specific CARs (Lamers et al., Biochem Soc Trans (2016) 44(3):951-959), FR-a-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10): 1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21 (14):3149-3159), IL13Rα2-specific CARs (Brown et al., Clin Cancer Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5): 1059-1070), VEGF-R-specific CARs (Chinnasamy et al., Cancer Res (2016) 22(2):436-447), FAP-specific CARs (Wang et al., Cancer Immunol Res (2014) 2(2): 154-166), MSLN-specific CARs (Moon et al., Clin Cancer Res (2011) 17(14):4719-30), and CD19-specific CARs (Axicabtagene ciloleucel (Yescarta®) and Tisagenlecleucel (Kymriah®)). See also, Li et al., J Hematol and Oncol (2018) 11(22), reviewing clinical trials of tumor-specific CARs. In some embodiments, a CAR is an anti-CD19 CAR.

As used herein, the term “CD16” refers to a receptor (FcγRIII) for the Fc portion of immunoglobulin G, and it is involved in the removal of antigen-antibody complexes from the circulation, as well as other antibody-dependent responses.

As used herein, the term “IL-15/IL15RA” or “Interleukin-15” (IL-15) refers to a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells. IL-15 Receptor alpha (IL15RA) specifically binds IL-15 with very high affinity, and is capable of binding IL-15 independently of other subunits. It is suggested that this property allows IL-15 to be produced by one cell, endocytosed by another cell, and then presented to a third party cell. IL15RA is reported to enhance cell proliferation and expression of apoptosis inhibitor BCL2L1/BCL2-XL and BCL2. Exemplary sequences of IL-15 are provided in NG_029605.2, and exemplary sequences of IL-15RA are provided in NM_002189.4. In some embodiments, the IL-15R variant is a constitutively active IL-15R variant. In some embodiments, the constitutively active IL-15R variant is a fusion between IL-15R and an IL-15R agonist, e.g., an IL-15 protein or IL-15R-binding fragment thereof. In some embodiments, the IL-15R agonist is IL-15, or an IL-15R-binding variant thereof. Exemplary suitable IL-15R variants include, without limitation, those described, e.g., in Mortier E et al, 2006; The Journal of Biological Chemistry 2006 281: 1612-1619; or in Bessard-A et al., Mol Cancer Ther. 2009 September; 8(9):2736-45, the entire contents of each of which are incorporated by reference herein.

As used herein, the term “IL-12” refers to interleukin-12, a cytokine that acts on T and natural killer cells. In some embodiments, a genetically engineered stem cell and/or progeny cell comprises a genetic modification that leads to expression of one or more of an interleukin 12 (IL12) pathway agonist, e.g., IL-12, interleukin 12 receptor (IL-12R) or a variant thereof (e.g., a constitutively active variant of IL-12R, e.g., an IL-12R fused to an IL-12R agonist (IL-12RA)).

As used herein, the term “CD47,” also sometimes referred to as “integrin associated protein” (IAP), refers to a transmembrane protein that in humans is encoded by the CD47 gene. CD47 belongs to the immunoglobulin superfamily, partners with membrane integrins, and also binds the ligands thrombospondin-1 (TSP-1) and signal-regulatory protein alpha (SIRPα). CD47 acts as a signal to macrophages that allows CD47-expressing cells to escape macrophage attack. See, e.g., Deuse-T, et al., Nature Biotechnology 2019 37: 252-258, the entire contents of which are incorporated herein by reference. In some embodiments, a CD47 gene comprises on or more mutations known to alter CD47 function.

In some embodiments, a CD47 nucleic acid sequence encoding a transgenic CD47 gene may be fused to one or more non-CD47 gene derived coding sequences. In some embodiments, a CD47 coding sequence may be codon-optimized.

In some embodiments, a CD47 transgene comprises or is SEQ ID NO: 1183. In some embodiments, a CD47 transgene comprises a coding sequence that is 60%, 65%, 70%, 75%, 80%, 85%, 90%, 95%, or 99% identical to SEQ ID NO: 1183.

CD47 nucleic acid sequence
SEQ ID NO: 1183
ATGTGGCCCCTGGTAGCGGCGCTGTTGCTGGGCTCGGCGTGCTGC
GGATCAGCTCAGCTACTATTTAATAAAACAAAATCTGTAGAATTC
ACGTTTTGTAATGACACTGTCGTCATTCCATGCTTTGTTACTAAT
ATGGAGGCACAAAACACTACTGAAGTATACGTAAAGTGGAAATTT
AAAGGAAGAGATATTTACACCTTTGATGGAGCTCTAAACAAGTCC
ACTGTCCCCACTGACTTTAGTAGTGCAAAAATTGAAGTCTCACAA
TTACTAAAAGGAGATGCCTCTTTGAAGATGGATAAGAGTGATGCT
GTCTCACACACAGGAAACTACACTTGTGAAGTAACAGAATTAACC
AGAGAAGGTGAAACGATCATCGAGCTAAAATATCGTGTTGTTTCA
TGGTTTTCTCCAAATGAAAATATTCTTATTGTTATTTTCCCAATT
TTTGCTATACTCCTGTTCTGGGGACAGTTTGGTATTAAAACACTT
AAATATAGATCCGGTGGTATGGATGAGAAAACAATTGCTTTACTT
GTTGCTGGACTAGTGATCACTGTCATTGTCATTGTTGGAGCCATT
CTTTTCGTCCCAGGTGAATATTCATTAAAGAATGCTACTGGCCTT
GGTTTAATTGTGACTTCTACAGGGATATTAATATTACTTCACTAC
TATGTGTTTAGTACAGCGATTGGATTAACCTCCTTCGTCATTGCC
ATATTGGTTATTCAGGTGATAGCCTATATCCTCGCTGTGGTTGGA
CTGAGTCTCTGTATTGCGGCGTGTATACCAATGCATGGCCCTCTT
CTGATTTCAGGTTTGAGTATCTTAGCTCTAGCACAATTACTTGGA
CTAGTTTATATGAAATTTGTGGCTTCCAATCAGAAGACTATACAA
CCTCCTAGGAATAAC

In some embodiments, a CD47 transgenic amino acid sequence comprises or is SEQ ID NO: 1184. In some embodiments, a CD47 amino acid sequence comprises a coding sequence that is 60%, 65%, 70%, 75%, 80%, 85%, 90%, 95%, or 99% identical to SEQ ID NO: 1184.

CD47 amino acid sequence
SEQ ID NO: 1184
MWPLVAALLLGSACCGSAQLLENKTKSVEFTFCNDTVVIPCFVIN
MEAQNTTEVYVKWKFKGRDIYTFDGALNKSTVPTDFSSAKIEVSQ
LLKGDASLKMDKSDAVSHIGNYTCEVTELTREGETIIELKYRVVS
WFSPNENILIVIFPIFAILLFWGQFGIKTLKYRSGGMDEKTIALL
VAGLVITVIVIVGAILFVPGEYSLKNATGLGLIVTSTGILILLHY
YVFSTAIGLISFVIAILVIQVIAYILAVVGLSLCIAACIPMHGPL
LISGLSILALAQLLGLVYMKFVASNQKTIQPPRNN

In some embodiments, a CD19 CAR nucleic acid sequence encoding a transgenic CD19 gene may be fused to one or more non-CD19 CAR gene derived coding sequences. In some embodiments, a CD19 CAR coding sequence may be codon-optimized.

In some embodiments, a CD19 CAR transgene comprises or is SEQ ID NO: 1232. In some embodiments, a CD19 CAR transgene comprises a coding sequence that is 60%, 65%, 70%, 75%, 80%, 85%, 90%, 95%, or 99% identical to SEQ ID NO: 1232.

CD19 CAR nucleic acid sequence
SEQ ID NO: 1232
ATGCTTCTCCTGGTGACAAGCCTTCTGCTCTGTGAGTTACCACAC
CCAGCATTCCTCCTGATCCCAGACATCCAGATGACACAGACTACA
TCCTCCCTGTCTGCCTCTCTGGGAGACAGAGTCACCATCAGTTGC
AGGGCAAGTCAGGACATTAGTAAATATTTAAATTGGTATCAGCAG
AAACCAGATGGAACTGTTAAACTCCTGATCTACCATACATCAAGA
TTACACTCAGGAGTCCCATCAAGGTTCAGTGGCAGTGGGTCTGGA
ACAGATTATTCTCTCACCATTAGCAACCTGGAGCAAGAAGATATT
GCCACTTACTTTTGCCAACAGGGTAATACGCTTCCGTACACGTTC
GGAGGGGGGACTAAGTTGGAAATAACAGGCTCCACCTCTGGATCC
GGCAAGCCCGGATCTGGCGAGGGATCCACCAAGGGCGAGGTGAAA
CTGCAGGAGTCAGGACCTGGCCTGGTGGCGCCCTCACAGAGCCTG
TCCGTCACATGCACTGTCTCAGGGGTCTCATTACCCGACTATGGT
GTAAGCTGGATTCGCCAGCCTCCACGAAAGGGTCTGGAGTGGCTG
GGAGTAATATGGGGTAGTGAAACCACATACTATAATTCAGCTCTC
AAATCCAGACTGACCATCATCAAGGACAACTCCAAGAGCCAAGTT
TTCTTAAAAATGAACAGTCTGCAAACTGATGACACAGCCATTTAC
TACTGTGCCAAACATTATTACTACGGTGGTAGCTATGCTATGGAC
TACTGGGGTCAAGGAACCTCAGTCACCGTCTCCTCAGCGGCCGCA
ATTGAAGTTATGTATCCTCCTCCTTACCTAGACAATGAGAAGAGC
AATGGAACCATTATCCATGTGAAAGGGAAACACCTTTGTCCAAGT
CCCCTATTTCCCGGACCTTCTAAGCCCTTTTGGGTGCTGGTGGTG
GTTGGGGGAGTCCTGGCTTGCTATAGCTTGCTAGTAACAGTGGCC
TTTATTATTTTCTGGGTGAGGAGTAAGAGGAGCAGGCTCCTGCAC
AGTGACTACATGAACATGACTCCCCGCCGCCCCGGGCCCACCCGC
AAGCATTACCAGCCCTATGCCCCACCACGCGACTTCGCAGCCTAT
CGCTCCAGAGTGAAGTTCAGCAGGAGCGCAGACGCCCCCGCGTAC
CAGCAGGGCCAGAACCAGCTCTATAACGAGCTCAATCTAGGACGA
AGAGAGGAGTACGATGTTTTGGACAAGAGACGTGGCCGGGACCCT
GAGATGGGGGGAAAGCCGAGAAGGAAGAACCCTCAGGAAGGCCTG
TACAATGAACTGCAGAAAGATAAGATGGCGGAGGCCTACAGTGAG
ATTGGGATGAAAGGCGAGCGCCGGAGGGGCAAGGGGCACGATGGC
CTTTACCAGGGTCTCAGTACAGCCACCAAGGACACCTACGACGCC
CTTCACATGCAGGCCCTGCCCCCTCGC

In some embodiments, a CD19 CAR transgenic amino acid sequence comprises or is SEQ ID NO: 1233. In some embodiments, a CD19 CAR amino acid sequence comprises a coding sequence that is 60%, 65%, 70%, 75%, 80%, 85%, 90%, 95%, or 99% identical to SEQ ID NO: 1233.

CD19 CAR amino acid sequence
SEQ ID NO: 1233
MLLLVTSLLLCELPHPAFLLIPDIQMTQTTSSLSASLGDRVTISC
RASQDISKYLNWYQQKPDGTVKLLIYHTSRLHSGVPSRFSGSGSG
TDYSLIISNLEQEDIATYFCQQGNILPYTFGGGTKLEITGSTSGS
GKPGSGEGSTKGEVKLQESGPGLVAPSQSLSVTCTVSGVSLPDYG
VSWIRQPPRKGLEWLGVIWGSETTYYNSALKSRLTIIKDNSKSQV
FLKMNSLQTDDTAIYYCAKHYYYGGSYAMDYWGQGTSVTVSSAAA
IEVMYPPPYLDNEKSNGTIIHVKGKHLCPSPLFPGPSKPFWVLVV
VGGVLACYSLLVTVAFIIFWVRSKRSRLLHSDYMNMTPRRPGPTR
KHYQPYAPPRDEAAYRSRVKFSRSADAPAYQQGQNQLYNELNLGR
REEYDVLDKRRGRDPEMGGKPRRKNPQEGLYNELQKDKMAEAYSE
IGMKGERRRGKGHDGLYQGLSTATKDTYDALHMQALPPR

### Donor Templates

In some embodiments, the present disclosure provides a donor template comprising a knock-in cassette with an exogenous coding sequence for a gene product of interest in frame with and downstream (3′) of an exogenous coding sequence or partial coding sequence of an essential gene, wherein the essential gene encodes a gene product that is required for survival, proliferation, and/or development of the cell.

In one aspect the present disclosure provides an impetus for designing donor templates comprising a knock-in cassette with an exogenous coding sequence for a gene product of interest in frame with and upstream (5′) of an exogenous coding sequence or partial coding sequence of an essential gene, wherein the essential gene encodes a gene product that is required for survival, proliferation, and/or development of the cell; see e.g., FIG. 19D.

In some embodiments, the donor template is for use in editing the genome of a cell by homology-directed repair (HDR).

Donor template design is described in detail in the literature, for instance in PCT Publication No. WO2016/073990A1. Donor templates can be single-stranded or double-stranded and can be used to facilitate HDR-based repair of double-strand breaks (DSBs), and are particularly useful for inserting a new sequence into the target sequence, or replacing the target sequence altogether. In some embodiments, the donor template is a donor DNA template. In some embodiments the donor DNA template is double-stranded.

Whether single-stranded or double stranded, donor templates generally include regions that are homologous to regions of DNA within or near (e.g., flanking or adjoining) a target sequence to be cleaved. These homologous regions are referred to herein as “homology arms.” and are illustrated schematically below relative to the knock-in cassette (which may be separated from one or both of the homology arms by additional spacer sequences that are not shown):
[5′ homology arm]-[knock-in cassette]-[3′ homology arm].

The homology arms can have any suitable length (including 0 nucleotides if only one homology arm is used), and 5′ and 3′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements.

A donor template can be a nucleic acid vector, such as a viral genome or circular double-stranded DNA, e.g., a plasmid. Nucleic acid vectors comprising donor templates can include other coding or non-coding elements. For example, a donor template nucleic acid can be delivered as part of a viral genome (e.g., in an AAV, adenoviral, Sendai virus, or lentiviral genome) that includes certain genomic backbone elements (e.g., inverted terminal repeats, in the case of an AAV genome). In some embodiments, a donor template is comprised in a plasmid that has not been linearized. In some embodiments, a donor template is comprised in a plasmid that has been linearized. In some embodiments, a donor template is comprised within a linear dsDNA fragment. In some embodiments, a donor template nucleic acid can be delivered as part of an AAV genome. In some embodiments, a donor template nucleic acid can be delivered as a single stranded oligo donor (ssODN), for example, as a long multi-kb ssODN derived from m13 phage synthesis, or alternatively, short ssODNs, e.g., that comprise small genes of interest, tags, and/or probes. In some embodiments, a donor template nucleic acid can be delivered as a Doggybone™ DNA (dbDNA™) template. In some embodiments, a donor template nucleic acid can be delivered as a DNA minicircle. In some embodiments, a donor template nucleic acid can be delivered as an Integration-deficient Lentiviral Particle (IDLV). In some embodiments, a donor template nucleic acid can be delivered as a MMLV-derived retrovirus. In some embodiments, a donor template nucleic acid can be delivered as a piggyBac™ sequence. In some embodiments, a donor template nucleic acid can be delivered as a replicating EBNA1 episome.

In certain embodiments, the 5′ homology arm may be about 25 to about 1,000 base pairs in length, e.g., at least about 100, 200, 400, 600, or 800 base pairs in length. In certain embodiments, the 5′ homology arm comprises about 50 to 800 base pairs, e.g., 100 to 800, 200 to 800, 400 to 800, 400 to 600, or 600 to 800 base pairs. In certain embodiments, the 3′ homology arm may be about 25 to about 1,000 base pairs in length, e.g., at least about 100, 200, 400, 600, or 800 base pairs in length. In certain embodiments, the 3′ homology arm comprises about 50 to 800 base pairs, e.g., 100 to 800, 200 to 800, 400 to 800, 400 to 600, or 600 to 800 base pairs. In certain embodiments, the 5′ and 3′ homology arms are symmetrical in length. In certain embodiments, the 5′ and 3′ homology arms are asymmetrical in length.

In certain embodiments, a 5′ homology arm is less than about 3,000 base pairs, less than about 2,900 base pairs, less than about 2,800 base pairs, less than about 2,700 base pairs, less than about 2,600 base pairs, less than about 2,500 base pairs, less than about 2,400 base pairs, less than about 2.300 base pairs, less than about 2,200 base pairs, less than about 2.100 base pairs, less than about 2,000 base pairs, less than about 1,900 base pairs, less than about 1,800 base pairs, less than about 1,700 base pairs, less than about 1,600 base pairs, less than about 1,500 base pairs, less than about 1,400 base pairs, less than about 1,300 base pairs, less than about 1,200 base pairs, less than about 1,100 base pairs, less than about 1,000 base pairs, less than about 900 base pairs, less than about 800 base pairs, less than about 700 base pairs, less than about 600 base pairs, less than about 500 base pairs, or less than about 400 base pairs.

In certain embodiments, e.g., where a viral vector is utilized to introduce a knock-in cassette through a method described herein, a 5′ homology arm is less than about 1,000 base pairs, less than about 900 base pairs, less than about 800 base pairs, is less than about 700 base pairs, less than about 600 base pairs, less than about 500 base pairs, less than about 400 base pairs, or less than about 300 base pairs. In certain embodiments, e.g., where a viral vector is utilized to introduce a knock-in cassette through a method described herein, a 5′ homology arm is about 400-600 base pairs, e.g., about 500 base pairs.

In certain embodiments, a 3′ homology arm is less than about 3,000 base pairs, less than about 2,900 base pairs, less than about 2,800 base pairs, less than about 2,700 base pairs, less than about 2,600 base pairs, less than about 2,500 base pairs, less than about 2,400 base pairs, less than about 2,300 base pairs, less than about 2,200 base pairs, less than about 2,100 base pairs, less than about 2,000 base pairs, less than about 1,900 base pairs, less than about 1,800 base pairs, less than about 1,700 base pairs, less than about 1,600 base pairs, less than about 1,500 base pairs, less than about 1,400 base pairs, less than about 1,300 base pairs, less than about 1,200 base pairs, less than about 1,100 base pairs, less than 1,000 base pairs, less than about 900 base pairs, less than about 800 base pairs, less than about 700 base pairs, less than about 600 base pairs, less than about 500 base pairs, or less than about 400 base pairs.

In certain embodiments, e.g., where a viral vector is utilized to introduce a knock-in cassette through a method described herein, a 3′ homology arm is less than about 1,000 base pairs, less than about 900 base pairs, less than about 800 base pairs, less than about 700 base pairs, less than about 600 base pairs, less than about 500 base pairs, less than about 400 base pairs, or less than about 300 base pairs. In certain embodiments, e.g., where a viral vector is utilized to introduce a knock-in cassette through a method described herein, a 3′ homology arm is about 400-600 base pairs, e.g., about 500 base pairs.

In certain embodiments, the 5′ and 3′ homology arms flank the break and are less than 100, 75, 50, 25, 15, 10 or 5 base pairs away from an edge of the break. In certain embodiments, the 5′ and 3′ homology arms flank an endogenous stop codon. In certain embodiments, the 5′ and 3′ homology arms flank a break located within about 500 base pairs (e.g., about 500 base pairs, about 450 base pairs, about 400 base pairs, about 350 base pairs, about 300 base pairs, about 250 base pairs, about 200 base pairs, about 150 base pairs, about 100 base pairs, about 50 base pairs, or about 25 base pairs) upstream (5′) of an endogenous stop codon, e.g., the stop codon of an essential gene. In certain embodiments, the 5′ homology arm encompasses an edge of the break.

Certain donor templates are also described in, e.g., WO2021/226151.

### Essential Genes

An essential gene can be any gene that is essential for the survival, the proliferation, and/or the development of the cell. In some embodiments, an essential gene is a housekeeping gene that is essential for survival of all cell types, e.g., a gene listed in Table 13. See also other housekeeping genes discussed in Eisenberg. Trends in Gen. 2014; 30(3): 119-20 and Moein et al., Adv. Biomed Res. 2017; 6:15; see also the essential genes discussed in Yilmaz et al., Nat. Cell Biol. 2018; 20:610-619 the entire contents of which are incorporated herein by reference.

In some embodiments the essential gene is GAPDH and the DNA nuclease causes a break in exon 9, e.g., a double-strand break. In some embodiments the essential gene is TBP and the DNA nuclease causes a break in exon 7, or exon 8, e.g., a double-strand break. In some embodiments the essential gene is E2F4 and the DNA nuclease causes a break in exon 10, e.g., a double-strand break. In some embodiments the essential gene is G6PD and the DNA nuclease causes a break in exon 13, e.g., a double-strand break. In some embodiments the essential gene is KIF11 and the DNA nuclease causes a break in exon 22, e.g., a double-strand break.

The gene symbols used in herein are based on those found in the Human Gene Naming Committee (HGNC) which is searchable on the world-wide web at genenames.org. Ensembl IDs are provided for each gene symbol and are searchable world-wide web at ensembl.org.

The genes provided herein are non-limiting examples of essential genes. Although additional essential genes will be apparent to the skilled artisan based on the knowledge in the art, the suitability of a particular gene for use according to the present disclosure can be determined, e.g., as discussed herein. For example, in some embodiments, a particular essential gene can be selected by analysis of potential off-target sites elsewhere in the genome. In some embodiments, only essential genes with one or more gRNA target sites that are unique in the human genome are selected for methods described herein. In some embodiments, only essential genes with one or more gRNA target sites that are found in only one other locus in the human genome are selected for methods described herein. In some embodiments, only essential genes with one or more gRNA target sites found in only two other loci in the human genome are selected for methods described herein.

TABLE 13
Exemplary housekeeping genes

| | Gene | | Gene |
|---|---|---|---|
| Ensembl ID | Symbol | Ensembl ID | Symbol |
| ENSG00000075624 | ACTB | ENSG00000231500 | RPS18 |
| ENSG00000116459 | ATP5F1 | ENSG00000112592 | TBP |
| ENSG00000166710 | B2M | ENSG00000072274 | TFRC |
| ENSG00000111640 | GAPDH | ENSG00000164924 | YWHAZ |
| ENSG00000169919 | GUSB | ENSG00000089157 | RPLP0 |
| ENSG00000165704 | HPRT1 | ENSG00000142541 | RPL13A |
| ENSG00000102144 | PGK1 | ENSG00000147604 | RPL7 |
| ENSG00000196262 | PPIA | ENSG00000205250 | E2F4 |
| ENSG00000138160 | KIF11 | ENSG00000160211 | G6PD |

### Knock-In Cassette

In some embodiments, a knock-in cassette within the donor template comprises an exogenous coding sequence for the gene product of interest in frame with and downstream (3′) of an exogenous coding sequence or partial coding sequence of the essential gene. In some embodiments, a knock-in cassette within a donor template comprises an exogenous coding sequence for the gene product of interest in frame with and upstream (5′) of an exogenous coding sequence or partial coding sequence of an essential gene. In some embodiments, the knock-in cassette is a polycistronic knock-in cassette. In some embodiments, the knock-in cassette is a bicistronic knock-in cassette. In some embodiment the knock-in cassette does not comprise a reporter gene, e.g., a fluorescent reporter gene or an antibiotic resistance gene.

In some embodiments, a single essential gene locus will be targeted by two knock-in cassettes comprising different “cargo” sequences. In some embodiments, one allele will incorporate one knock-in cassette, while the other allele will incorporate the other knock-in cassette. In some embodiments, a gRNA utilized to generate an appropriate DNA break may be the same for each of the two different knock-in cassettes. In some embodiments, gRNAs utilized to generate appropriate DNA breaks for each of the two different knock-in cassettes may be different, such that the “cargo” sequence is incorporated at a different position for each allele. In some embodiments, such a different position for each allele may still be within the ultimate exon's coding region. In some embodiments, such a different position for each allele may be within the penultimate exon (second to last), and/or ultimate (last) exon's coding region. In some embodiments, such a different position for at least one of the alleles may be within the first exon. In some embodiments, such a different position for at least one of the alleles may be within the first or second exon.

In order to properly restore the essential gene coding region in the genetically modified cell (so that a functioning gene product is produced) the knock-in cassette does not need to comprise an exogenous coding sequence that corresponds to the entire coding sequence of the essential gene. Indeed, depending on the location of the break in the endogenous coding sequence of the essential gene it may be possible to restore the essential gene by providing a knock-in cassette that comprises a partial coding sequence of the essential gene, e.g., that corresponds to a portion of the endogenous coding sequence of the essential gene that spans the break and the entire region downstream of the break (minus the stop codon), and/or that corresponds to a portion of the endogenous coding sequence of the essential gene that spans the break and the entire region upstream of the break (up to and optionally including the start codon).

In order to minimize the size of the knock-in cassette it may in fact be advantageous, in some embodiments, to have the break located within the last 1500, 1000, 750, 500, 400, 300, 200, 100, or 50 base pairs of the endogenous coding sequence of the essential gene, i.e., towards the 3′ end of the coding sequence. In some embodiments, a base pair's location in a coding sequence may be defined 3′-to-5′ from an endogenous translational stop signal (e.g., a stop codon). In some embodiments, as used herein, an “endogenous coding sequence” can include both exonic and intronic base pairs, and refers to gene sequence occurring 5′ to an endogenous functional translational stop signal. In some embodiments, a break within an endogenous coding sequence comprises a break within one DNA strand. In some embodiments, a break within an endogenous coding sequence comprises a break within both DNA strands. In some embodiments, a break is located within the last 1000 base pairs of the endogenous coding sequence. In some embodiments, a break is located within the last 750 base pairs of the endogenous coding sequence. In some embodiments, a break is located within the last 600 base pairs of the endogenous coding sequence. In some embodiments, a break is located within the last 500 base pairs of the endogenous coding sequence. In some embodiments, a break is located within the last 400 base pairs of the endogenous coding sequence. In some embodiments, a break is located within the last 300 base pairs of the endogenous coding sequence. In some embodiments, a break is located within the last 250 base pairs of the endogenous coding sequence. In some embodiments, a break is located within the last 200 base pairs of the endogenous coding sequence. In some embodiments, a break is located within the last 150 base pairs of the endogenous coding sequence. In some embodiments, a break is located within the last 100 base pairs of the endogenous coding sequence. In some embodiments, a break is located within the last 75 base pairs of the endogenous coding sequence. In some embodiments, a break is located within the last 50 base pairs of the endogenous coding sequence. In some embodiments, a break is located within the last 21 base pairs of the endogenous coding sequence.

In some embodiments, the exogenous partial coding sequence of the essential gene in the knock-in cassette encodes a C-terminal fragment of a protein encoded by the essential gene, e.g., a fragment that is less than 500, 250, 150, 125, 100, 75, 50, 25, 20, 15 or 10 amino acids in length. In some embodiments, the exogenous partial coding sequence of the essential gene in the knock-in cassette is codon optimized. In some embodiments, the exogenous partial coding sequence of the essential gene in the knock-in cassette is codon optimized to eliminate at least one PAM site. In some embodiments, the exogenous partial coding sequence of the essential gene in the knock-in cassette is codon optimized to eliminate more than one PAM site. In some embodiments, the exogenous partial coding sequence of the essential gene in the knock-in cassette is codon optimized to eliminate all relevant nuclease specific PAM sites. In some embodiments, a C-terminal fragment of a protein encoded by the essential gene is about 140 amino acids in length. In some embodiments, a C-terminal fragment of a protein encoded by the essential gene is about 130 amino acids in length. In some embodiments, a C-terminal fragment of a protein encoded by the essential gene is about 120 amino acids in length. In some embodiments, the C-terminal fragment includes an amino acid sequence that is encoded by a region of the endogenous coding sequence of the essential gene that spans the break. In some embodiments, a C-terminal fragment includes an amino acid sequence that is encoded by a region of the endogenous coding sequence within 1 exon of the essential gene. In some embodiments, a C-terminal fragment includes an amino acid sequence that is encoded by a region of the endogenous coding sequence within 2 exons of the essential gene. In some embodiments, a C-terminal fragment includes an amino acid sequence that is encoded by a region of the endogenous coding sequence within 3 exons of the essential gene. In some embodiments, a C-terminal fragment includes an amino acid sequence that is encoded by a region of the endogenous coding sequence within 4 exons of the essential gene. In some embodiments, a C-terminal fragment includes an amino acid sequence that is encoded by a region of the endogenous coding sequence within 5 exons of the essential gene.

In some embodiments, the exogenous partial coding sequence of an essential gene in a knock-in cassette encodes a C-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette encodes a 20 amino acid C-terminal fragment of a protein encoded by an essential gene. In some embodiments, the exogenous partial coding sequence of an essential gene in a knock-in cassette encodes a 19 amino acid C-terminal fragment of a protein encoded by an essential gene. In some embodiments, the exogenous partial coding sequence of an essential gene in a knock-in cassette encodes an 18 amino acid C-terminal fragment of a protein encoded by an essential gene. In some embodiments, the exogenous partial coding sequence of an essential gene in a knock-in cassette encodes a 17 amino acid C-terminal fragment of a protein encoded by an essential gene. In some embodiments, the exogenous partial coding sequence of an essential gene in a knock-in cassette encodes a 16 amino acid C-terminal fragment of a protein encoded by an essential gene. In some embodiments, the exogenous partial coding sequence of an essential gene in a knock-in cassette encodes a 1 amino acid C-terminal fragment of a protein encoded by an essential gene.

In some embodiments, e.g., when the essential gene includes many exons as shown in the exemplary method of FIG. 19A, it may be advantageous to have the break within the last exon of the essential gene. In some embodiments, e.g., when the essential gene includes many exons as shown in the exemplary method of FIG. 19A, it may be advantageous to have the break within the penultimate exon of the essential gene. It is to be understood however that the present disclosure is not limited to any particular location for the break and that the available positions will vary depending on the nature and length of the essential gene and the length of the exogenous coding sequence for the gene product of interest. For example, for essential genes that include a few exons or when the gene product of interest is small it may be possible to locate the break in an upstream exon.

In order to minimize the size of the knock-in cassette it may in fact be advantageous, in some embodiments, to have the break located within the first 1500, 1000, 750, 500, 400, 300, 200, 100, or 50 base pairs of an endogenous coding sequence of the essential gene, i.e., starting from the 5′ end of a coding sequence. In some embodiments, a base pair's location in a coding sequence may be defined 5′-to-3′ from an endogenous translational start signal (e.g., a start codon). In some embodiments, as used herein, an “endogenous coding sequence” can include both exonic and intronic base pairs, and refers to gene sequence occurring 3′ to an endogenous functional translational start signal. In some embodiments, a break within an endogenous coding sequence comprises a break within one DNA strand. In some embodiments, a break within an endogenous coding sequence comprises a break within both DNA strands. In some embodiments, a break is located within the first 1000 base pairs of the endogenous coding sequence. In some embodiments, a break is located within the first 750 base pairs of the endogenous coding sequence. In some embodiments, a break is located within the first 600 base pairs of the endogenous coding sequence. In some embodiments, a break is located within the first 500 base pairs of the endogenous coding sequence. In some embodiments, a break is located within the first 400 base pairs of the endogenous coding sequence. In some embodiments, a break is located within the first 300 base pairs of the endogenous coding sequence. In some embodiments, a break is located within the first 250 base pairs of the endogenous coding sequence. In some embodiments, a break is located within the first 200 base pairs of the endogenous coding sequence. In some embodiments, a break is located within the first 150 base pairs of the endogenous coding sequence. In some embodiments, a break is located within the first 100 base pairs of the endogenous coding sequence. In some embodiments, a break is located within the first 75 base pairs of the endogenous coding sequence. In some embodiments, a break is located within the first 50 base pairs of the endogenous coding sequence. In some embodiments, a break is located within the first 21 base pairs of the endogenous coding sequence.

In some embodiments, the exogenous partial coding sequence of the essential gene in the knock-in cassette encodes an N-terminal fragment of a protein encoded by the essential gene, e.g., a fragment that is less than 500, 250, 150, 125, 100, 75, 50, 25, 20, 15 or 10 amino acids in length. In some embodiments, an N-terminal fragment of a protein encoded by the essential gene is about 140 amino acids in length. In some embodiments, an N-terminal fragment of a protein encoded by the essential gene is about 130 amino acids in length. In some embodiments, an N-terminal fragment of a protein encoded by the essential gene is about 120 amino acids in length. In some embodiments, an N-terminal fragment includes an amino acid sequence that is encoded by a region of the endogenous coding sequence of the essential gene that spans the break. In some embodiments, an N-terminal fragment includes an amino acid sequence that is encoded by a region of the endogenous coding sequence within 1 exon of the essential gene. In some embodiments, an N-terminal fragment includes an amino acid sequence that is encoded by a region of the endogenous coding sequence within 2 exons of the essential gene. In some embodiments, an N-terminal fragment includes an amino acid sequence that is encoded by a region of the endogenous coding sequence within 3 exons of the essential gene. In some embodiments, an N-terminal fragment includes an amino acid sequence that is encoded by a region of the endogenous coding sequence within 4 exons of the essential gene. In some embodiments, an N-terminal fragment includes an amino acid sequence that is encoded by a region of the endogenous coding sequence within 5 exons of the essential gene.

In some embodiments, the exogenous partial coding sequence of an essential gene in a knock-in cassette encodes an N-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette encodes a 20 amino acid N-terminal fragment of a protein encoded by an essential gene. In some embodiments, the exogenous partial coding sequence of an essential gene in a knock-in cassette encodes a 19 amino acid N-terminal fragment of a protein encoded by an essential gene. In some embodiments, the exogenous partial coding sequence of an essential gene in a knock-in cassette encodes an 18 amino acid N-terminal fragment of a protein encoded by an essential gene. In some embodiments, the exogenous partial coding sequence of an essential gene in a knock-in cassette encodes a 17 amino acid N-terminal fragment of a protein encoded by an essential gene. In some embodiments, the exogenous partial coding sequence of an essential gene in a knock-in cassette encodes a 16 amino acid N-terminal fragment of a protein encoded by an essential gene. In some embodiments, the exogenous partial coding sequence of an essential gene in a knock-in cassette encodes a 1 amino acid N-terminal fragment of a protein encoded by an essential gene.

In some embodiments, the exogenous coding sequence or partial coding sequence of the essential gene in the knock-in cassette is less than 100% identical to the corresponding endogenous coding sequence of the essential gene of the cell, e.g., less than 99%, 98%, 97%, 96%, 95%, 94%, 93%, 92%, 91%, 90%, 85%, 80%, 75%, 70%, 65%, 60%, 55% or less than 50% (i.e., when the two sequences are aligned using a standard pairwise sequence alignment tool that maximizes the alignment between the corresponding sequences). For example, in some embodiments, the exogenous coding sequence or partial coding sequence of the essential gene in the knock-in cassette is codon optimized relative to the corresponding endogenous coding sequence of the essential gene of the cell, e.g., to prevent further binding of a nuclease to the target site. Alternatively or additionally it may be codon optimized to reduce the likelihood of recombination after integration of the knock-in cassette into the genome of the cell and/or to increase expression of the gene product of the essential gene and/or the gene product of interest after integration of the knock-in cassette into the genome of the cell.

In some embodiments, a knock-in cassette comprises one or more nucleotides or base pairs that differ (e.g., are mutations) relative to an endogenous knock-in site. In some embodiments, such mutations in a knock-in cassette provide resistance to cutting by a nuclease. In some embodiments, such mutations in a knock-in cassette prevent a nuclease from cutting the target loci following homologous recombination. In some embodiments, such mutations in a knock-in cassette occur within one or more coding and/or non-coding regions of a target gene. In some embodiments, such mutations in a knock-in cassette are silent mutations. In some embodiments, such mutations in a knock-in cassette are silent and/or missense mutations.

In some embodiments, such mutations in a knock-in cassette occur within a target protospacer motif and/or a target protospacer adjacent motif (PAM) site. In some embodiments, a knock-in cassette includes a target protospacer motif and/or a PAM site that are saturated with silent mutations. In some embodiments, a knock-in cassette includes a target protospacer motif and/or a PAM site that are approximately 30%, 40%, 50%, 60%, 70%, 80%, or 90% saturated with silent mutations. In some embodiments, a knock-in cassette includes a target protospacer motif and/or a PAM site that are saturated with silent and/or missense mutations. In some embodiments, a knock-in cassette includes a target protospacer motif and/or a PAM site that comprise at least one mutation, at least 2 mutations, at least 3 mutations, at least 4 mutations, at least 5 mutations, at least 6 mutations, at least 7 mutations, at least 8 mutations, at least 9 mutations, at least 10 mutations, at least 11 mutations, at least 12 mutations, at least 13 mutations, at least 14 mutations, or at least 15 mutations.

In some embodiments, certain codons encoding certain amino acids in a target site cannot be mutated through codon-optimization without losing some portion of an endogenous proteins natural function. In some embodiments, certain codons encoding certain amino acids in a target site cannot be mutated through codon-optimization.

In some embodiments, the knock-in cassette is codon optimized in only a portion of the coding sequence. For example, in some embodiments, a knock-in cassette encodes a C-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette that has been codon optimized encodes a 20 amino acid C-terminal fragment of a protein encoded by an essential gene. In some embodiments, the exogenous partial coding sequence of an essential gene in a knock-in cassette that has been codon optimized encodes a 19 amino acid C-terminal fragment of a protein encoded by an essential gene. In some embodiments, the exogenous partial coding sequence of an essential gene in a knock-in cassette that has been codon optimized encodes an 18 amino acid C-terminal fragment of a protein encoded by an essential gene. In some embodiments, the exogenous partial coding sequence of an essential gene in a knock-in cassette that has been codon optimized encodes a 17 amino acid C-terminal fragment of a protein encoded by an essential gene. In some embodiments, the exogenous partial coding sequence of an essential gene in a knock-in cassette that has been codon optimized encodes a 16 amino acid C-terminal fragment of a protein encoded by an essential gene. In some embodiments, the exogenous partial coding sequence of an essential gene in a knock-in cassette that has been codon optimized encodes a 15 amino acid C-terminal fragment of a protein encoded by an essential gene. In some embodiments, the exogenous partial coding sequence of an essential gene in a knock-in cassette that has been codon optimized encodes a 14 amino acid C-terminal fragment of a protein encoded by an essential gene. In some embodiments, the exogenous partial coding sequence of an essential gene in a knock-in cassette that has been codon optimized encodes a 13 amino acid C-terminal fragment of a protein encoded by an essential gene. In some embodiments, the exogenous partial coding sequence of an essential gene in a knock-in cassette that has been codon optimized encodes a 12 amino acid C-terminal fragment of a protein encoded by an essential gene. In some embodiments, the exogenous partial coding sequence of an essential gene in a knock-in cassette that has been codon optimized encodes an 11 amino acid C-terminal fragment of a protein encoded by an essential gene. In some embodiments, the exogenous partial coding sequence of an essential gene in a knock-in cassette that has been codon optimized encodes a 10 amino acid C-terminal fragment of a protein encoded by an essential gene. In some embodiments, the exogenous partial coding sequence of an essential gene in a knock-in cassette that has been codon optimized encodes a 9 amino acid C-terminal fragment of a protein encoded by an essential gene. In some embodiments, the exogenous partial coding sequence of an essential gene in a knock-in cassette that has been codon optimized encodes an 8 amino acid C-terminal fragment of a protein encoded by an essential gene. In some embodiments, the exogenous partial coding sequence of an essential gene in a knock-in cassette that has been codon optimized encodes a 7 amino acid C-terminal fragment of a protein encoded by an essential gene. In some embodiments, the exogenous partial coding sequence of an essential gene in a knock-in cassette that has been codon optimized encodes a 6 amino acid C-terminal fragment of a protein encoded by an essential gene. In some embodiments, the exogenous partial coding sequence of an essential gene in a knock-in cassette that has been codon optimized encodes a 5 amino acid C-terminal fragment of a protein encoded by an essential gene. In some embodiments, the exogenous partial coding sequence of an essential gene in a knock-in cassette that has been codon optimized encodes an amino acid C-terminal fragment that is less than 5 amino acids of a protein encoded by an essential gene.

In some embodiments, the knock-in cassette is codon optimized in only a portion of the coding sequence. For example, in some embodiments, a knock-in cassette encodes an N-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette that has been codon optimized encodes a 20 amino acid N-terminal fragment of a protein encoded by an essential gene. In some embodiments, the exogenous partial coding sequence of an essential gene in a knock-in cassette that has been codon optimized encodes a 19 amino acid N-terminal fragment of a protein encoded by an essential gene. In some embodiments, the exogenous partial coding sequence of an essential gene in a knock-in cassette that has been codon optimized encodes an 18 amino acid N-terminal fragment of a protein encoded by an essential gene. In some embodiments, the exogenous partial coding sequence of an essential gene in a knock-in cassette that has been codon optimized encodes a 17 amino acid N-terminal fragment of a protein encoded by an essential gene. In some embodiments, the exogenous partial coding sequence of an essential gene in a knock-in cassette that has been codon optimized encodes a 16 amino acid N-terminal fragment of a protein encoded by an essential gene. In some embodiments, the exogenous partial coding sequence of an essential gene in a knock-in cassette that has been codon optimized encodes a 15 amino acid N-terminal fragment of a protein encoded by an essential gene. In some embodiments, the exogenous partial coding sequence of an essential gene in a knock-in cassette that has been codon optimized encodes a 14 amino acid N-terminal fragment of a protein encoded by an essential gene. In some embodiments, the exogenous partial coding sequence of an essential gene in a knock-in cassette that has been codon optimized encodes a 13 amino acid N-terminal fragment of a protein encoded by an essential gene. In some embodiments, the exogenous partial coding sequence of an essential gene in a knock-in cassette that has been codon optimized encodes a 12 amino acid N-terminal fragment of a protein encoded by an essential gene. In some embodiments, the exogenous partial coding sequence of an essential gene in a knock-in cassette that has been codon optimized encodes an 11 amino acid N-terminal fragment of a protein encoded by an essential gene. In some embodiments, the exogenous partial coding sequence of an essential gene in a knock-in cassette that has been codon optimized encodes a 10 amino acid N-terminal fragment of a protein encoded by an essential gene. In some embodiments, the exogenous partial coding sequence of an essential gene in a knock-in cassette that has been codon optimized encodes a 9 amino acid N-terminal fragment of a protein encoded by an essential gene. In some embodiments, the exogenous partial coding sequence of an essential gene in a knock-in cassette that has been codon optimized encodes an 8 amino acid N-terminal fragment of a protein encoded by an essential gene. In some embodiments, the exogenous partial coding sequence of an essential gene in a knock-in cassette that has been codon optimized encodes a 7 amino acid N-terminal fragment of a protein encoded by an essential gene. In some embodiments, the exogenous partial coding sequence of an essential gene in a knock-in cassette that has been codon optimized encodes a 6 amino acid N-terminal fragment of a protein encoded by an essential gene. In some embodiments, the exogenous partial coding sequence of an essential gene in a knock-in cassette that has been codon optimized encodes a 5 amino acid N-terminal fragment of a protein encoded by an essential gene. In some embodiments, the exogenous partial coding sequence of an essential gene in a knock-in cassette that has been codon optimized encodes an amino acid N-terminal fragment that is less than 5 amino acids of a protein encoded by an essential gene.

In some embodiments, the knock-in cassette comprises one or more sequences encoding a linker peptide, e.g., between an exogenous coding sequence or partial coding sequence of the essential gene and a “cargo” sequence and/or a regulatory element described herein. Such linker peptides are known in the art, any of which can be included in a knock-in cassette described herein. In some embodiments, the linker peptide comprises the amino acid sequence GSG.

In some embodiments, the knock-in cassette comprises other regulatory elements such as a polyadenylation sequence, and optionally a 3′ UTR sequence, downstream of the exogenous coding sequence for the gene product of interest. If a 3′UTR sequence is present, the 3′UTR sequence is positioned 3′ of the exogenous coding sequence and 5′ of the polyadenylation sequence.

In some embodiments, the knock-in cassette comprises other regulatory elements such as a 5′ UTR and a start codon, upstream of the exogenous coding sequence for the gene product of interest. If a 5′UTR sequence is present, the 5′UTR sequence is positioned 5′ of the “cargo” sequence and/or exogenous coding sequence.

Certain knock-in cassettes are also described in, e.g., WO2021/226151.

### IRES and 2A Elements

In some embodiments, the knock-in cassette comprises a regulatory element that enables expression of the gene product encoded by the essential gene and the gene product of interest as separate gene products, e.g., an IRES or 2A element located between the exogenous coding sequence or partial coding sequence of the essential gene and the exogenous coding sequence for the gene product of interest.

In some embodiments, a knock-in cassette may comprise multiple gene products of interest (e.g., at least two gene products of interest). In some embodiments, gene products of interest may be separated by a regulatory element that enables expression of the at least two gene products of interest as more than one gene product, e.g., an IRES or 2A element located between the at least two coding sequences, facilitating creation of at least two peptide products.

Internal Ribosome Entry Site (IRES) elements are one type of regulatory element that are commonly used for this purpose. As is well known in the art, IRES elements allow for initiation of translation from an internal region of the mRNA and hence expression of two separate proteins from the same mRNA transcript. IRES was originally discovered in poliovirus RNA, where it promotes translation of the viral genome in eukaryotic cells. Since then, a variety of IRES sequences have been discovered-many from viruses, but also some from cellular mRNAs, e.g., see Mokrejs et al., Nucleic Acids Res. 2006; 34(Database issue):D125-D130.

2A elements are another type of regulatory element that are commonly used for this purpose. These 2A elements encode so-called “self-cleaving” 2A peptides which are short peptides (about 20 amino acids) that were first discovered in picornaviruses. The term “self-cleaving” is not entirely accurate, as these peptides are thought to function by making the ribosome skip the synthesis of a peptide bond at the C-terminus of a 2A element, leading to separation between the end of the 2A sequence and the next peptide downstream. The “cleavage” occurs between the Glycine (G) and Proline (P) residues found on the C-terminus meaning the upstream cistron, i.e., protein encoded by the essential gene will have a few additional residues from the 2A peptide added to the end, while the downstream cistron, i.e., gene product of interest will start with the Proline (P).

Table 14 below lists the four commonly used 2A peptides (an optional GSG sequence is sometimes added to the N-terminal end of the peptide to improve cleavage efficiency). There are many potential 2A peptides that may be suitable for methods and compositions described herein (see e.g., Luke et al., Occurrence, function and evolutionary origins of ‘2A-like’ sequences in virus genomes. J Gen Virol. 2008). Those skilled in the art know that the choice of specific 2A peptide for a particular knock-in cassette will ultimately depend on a number of factors such as cell type or experimental conditions. Those skilled in the art will recognize that nucleotide sequences encoding specific 2A peptides can vary while still encoding a peptide suitable for inducing a desired cleavage event.

TABLE 14
Exemplary IRES and 2A peptide
and nucleic acid sequences

| SEQ | | |
|---|---|---|
| ID | 2A | |
| NO: | peptide | Amino acid sequence |
| 1185 | T2A | EGRGSLLTCGDVEENPGP |
| 1186 | P2A | ATNFSLLKQAGDVEENPGP |
| 1187 | E2A | QCTNYALLKLAGDVESNPGP |
| 1188 | F2A | VKQTLNFDLLKLAGDVESNPGP |
| 1189 | T2A | GAGGGCAGAGGAAGTCTTCTAACAT |
| | | GCGGTGACGTGGAGGAGAATCCTGG |
| | | CCCG |
| 1190 | P2A | GGAAGCGGAGCTACTAACTTCAGCC |
| | | TGCTGAAGCAGGCTGGAGACGTGGA |
| | | GGAGAACCCTGGACCT |
| 1191 | E2A | CAGTGTACTAATTATGCTCTCTTGA |
| | | AATTGGCTGGAGATGTTGAGAGCAA |
| | | CCCTGGACCT |
| 1192 | F2A | GTGAAACAGACTTTGAATTTTGACC |
| | | TTCTCAAGTTGGCGGGAGACGTGGA |
| | | GTCCAACCCTGGACCT |
| 1193 | IRES | CCCCTCTCCCTCCCCCCCCCCTAAC |
| | | GTTACTGGCCGAAGCCGCTTGGAAT |
| | | AAGGCCGGTGTGCGTTTGTCTATAT |
| | | GTTATTTTCCACCATATTGCCGTCT |
| | | TTTGGCAATGTGAGGGCCCGGAAAC |
| | | CTGGCCCTGTCTTCTTGACGAGCAT |
| | | TCCTAGGGGTCTTTCCCCTCTCGCC |
| | | AAAGGAATGCAAGGTCTGTTGAATG |
| | | TCGTGAAGGAAGCAGTTCCTCTGGA |
| | | AGCTTCTTGAAGACAAACAACGTCT |
| | | GTAGCGACCCTTTGCAGGCAGCGGA |
| | | ACCCCCCACCTGGCGACAGGTGCCT |
| | | CTGCGGCCAAAAGCCACGTGTATAA |
| | | GATACACCTGCAAAGGCGGCACAAC |
| | | CCCAGTGCCACGTTGTGAGTTGGAT |
| | | AGTTGTGGAAAGAGTCAAATGGCTC |
| | | TCCTCAAGCGTATTCAACAAGGGGC |
| | | TGAAGGATGCCCAGAAGGTACCCCA |
| | | TTGTATGGGATCTGATCTGGGGCCT |
| | | CGGTGCACATGCTTTACATGTGTTT |
| | | AGTCGAGGTTAAAAAAACGTCTAGG |
| | | CCCCCCGAACCACGGGGACGTGGTT |
| | | TTCCTTTGAAAAACACGATGATAA |

### Exemplary Homology Arms (HA)

In certain embodiments, a donor template comprises a 5′ and/or 3′ homology arm homologous to region of a GAPDH locus. In some embodiments, a donor template comprises a 5′ homology arm comprising or consisting of the sequence of SEQ ID NO: 1194. In some embodiments, a 5′ homology arm comprises or consists of a sequence that is at least 85%, 90%, 95%, 98% or 99% identical to the sequence of SEQ ID NO: 1194. In some embodiments, a donor template comprises a 3′ homology arm comprising or consisting of the sequence of SEQ ID NO: 1195. In certain embodiments, a 3′ homology arm comprises or consists of a sequence that is at least 85%, 90%, 95%, 98% or 99% identical to the sequence of SEQ ID NO: 1195.

In some embodiments, a donor template comprises a 5′ homology arm comprising SEQ ID NO: 1194, and a 3′ homology arm comprising SEQ ID NO: 1195.

In some embodiments, a stretch of sequence flanking a nuclease cleavage site may be duplicated in both a 5′ and 3′ homology arm. In some embodiments, such a duplication is designed to optimize HDR efficiency. In some embodiments, one of the duplicated sequences may be codon optimized, while the other sequence is not codon optimized. In some embodiments, both of the duplicated sequences may be codon optimized. In some embodiments, codon optimization may remove a target PAM site. In some embodiments, a duplicated sequence may be no more than: 100 bp in length, 90 bp in length, 80 bp in length, 70 bp in length, 60 bp in length, 50 bp in length, 40 bp in length, 30 bp in length, or 20 bp in length.

exemplary 5′ HA for knock-in cassette
insertion at GAPDH locus
SEQ ID NO: 1194
GAAGACTGTGGATGGCCCCTCCGGGAAACTGTGGCGTGATGGCCG
CGGGGCTCTCCAGAACATCATCCCTGCCTCTACTGGCGCTGCCAA
GGCTGTGGGCAAGGTCATCCCTGAGCTGAACGGGAAGCTCACTGG
CATGGCCTTCCGTGTCCCCACTGCCAACGTGTCAGTGGTGGACCT
GACCTGCCGTCTAGAAAAACCTGCCAAATATGATGACATCAAGAA
GGTGGTGAAGCAGGCGTCGGAGGGCCCCCTCAAGGGCATCCTGGG
CTACACTGAGCACCAGGTGGTCTCCTCTGACTTCAACAGCGACAC
CCACTCCTCCACCTTTGACGCTGGGGCTGGCATTGCCCTCAACGA
CCACTTTGTCAAGCTCATTTCCTGGTATGTGGCTGGGGCCAGAGA
CTGGCTCTTAAAAAGTGCAGGGTCTGGCGCCCTCTGGTGGCTGGC
TCAGAAAAAGGGCCCTGACAACTCTTTACATCTTCTAGGTATGAC
AACGAGTTCGGATATAGCAATAGAGTGGTCGATCTGATGGCTCAT
ATGGCTAGCAAAGAG
exemplary 3′ HA for knock-in cassette
insertion at GAPDH locus
SEQ ID NO: 1195
ATTTGGCTACAGCAACAGGGTGGTGGACCTCATGGCCCACATGGC
CTCCAAGGAGTAAGACCCCTGGACCACCAGCCCCAGCAAGAGCAC
AAGAGGAAGAGAGAGACCCTCACTGCTGGGGAGTCCCTGCCACAC
TCAGTCCCCCACCACACTGAATCTCCCCTCCTCACAGTTGCCATG
TAGACCCCTTGAAGAGGGGAGGGGCCTAGGGAGCCGCACCTTGTC
ATGTACCATCAATAAAGTACCCTGTGCTCAACCAGTTACTTGTCC
TGTCTTATTCTAGGGTCTGGGGCAGAGGGGAGGGAAGCTGGGCTT
GTGTCAAGGTGAGACATTCTTGCTGGGGAGGGACCTGGTATGTTC
TCCTCAGACTGAGGGTAGGGCCTCCAAACAGCCTTGCTTGCTTCG
AGAACCATTTGCTTCCCGCTCAGACGTCTTGAGTGCTACAGGAAG
CTGGCACCACTACTTCAGAGAACAAGGCCTTTTCCTCTCCTCGCT
CCAGT

In some embodiments, a donor template comprises a 5′ and/or 3′ homology arm homologous to a region of a TBP locus. In some embodiments, a donor template comprises a 5′ homology arm comprising or consisting of the sequence of SEQ ID NO: 1196. In some embodiments, a 5′ homology arm comprises or consists of a sequence that is at least 85%, 90%, 95%, 98% or 99% identical to the sequence of SEQ ID NO: 1196. In some embodiments, a donor template comprises a 3′ homology arm comprising or consisting of the sequence of SEQ ID NO: 1197. In certain embodiments, a 3′ homology arm comprises or consists of a sequence that is at least 85%, 90%, 95%, 98% or 99% identical to the sequence of SEQ ID NO: 1197.

In some embodiments, a donor template comprises a 5′ homology arm comprising SEQ ID NO: 1196, and a 3′ homology arm comprising SEQ ID NO: 1197.

exemplary 5′ HA for knock-in cassette
insertion at TBP locus
SEQ ID NO: 1196
CTGACCACAGCTCTGCAAGCAGACTTCCATTTACAGTGAGGAGGT
GAGCATTGCATTGAACAAAAGATGGCGTTTTCACTTGGAATTAGT
TATCTGAAGCTTTAGGATTCCTCAGCAATATGATTATGAGACAAG
AAAGGAAGATTCAGAAATGAGTCTAGTTGAAGGCAGCAATTCAGA
GAAGAAGATTCAGTTGTTATCATTGCCGTCCTGCTTGGTTTATGG
CCTGGTTCAGGACCAAGGAGAGAAGTGTGAATACATGCCTCTTGA
GCTATAGAATGAGACGCTGGAGTCACTAAGATGATTTTTTAAAAG
TATTGTTTTATAAACAAAAATAAGATTGTGACAAGGGATTCCACT
ATTAATGTTTTCATGCCTGTGCCTTAATCTGACTGGGTATGGTGA
GAATTGTGCTTGCAGCTTTAAGGTAAGAATTTTACCATCTTAATA
TGTTAAGAAGTGCCATTTCAGTCTCTCATCTCTACTCCAACTTGT
CTTCTTAGGGGCTAAAGTGCGGGCCGAGATCTACGAGGCCTTCGA
GAATATCTACCCCATCCTGAAGGGCTTCAGAAAGACCACC
exemplary 3′ HA for knock-in
cassette insertion at TBP locus
SEQ ID NO: 1197
TAGGTGCTAAAGTCAGAGCAGAAATTTATGAAGCATTTGAAAACA
TCTACCCTATTCTAAAGGGATTCAGGAAGACGACGTAATGGCTCT
CATGTACCCTTGCCTCCCCCACCCCCTTCTTTTTTTTTTTTTAAA
CAAATCAGTTTGTTTTGGTACCTTTAAATGGTGGTGTTGTGAGAA
GATGGATGTTGAGTTGCAGGGTGTGGCACCAGGTGATGCCCTTCT
GTAAGTGCCCACCGCGGGATGCCGGGAAGGGGCATTATTTGTGCA
CTGAGAACACCGCGCAGCGTGACTGTGAGTTGCTCATACCGTGCT
GCTATCTGGGCAGCGCTGCCCATTTATTTATATGTAGATTTTAAA
CACTGCTGTTGACAAGTTGGTTTGAGGGAGAAAACTTTAAGTGTT
AAAGCCACCTCTATAATTGATTGGACTTTTTAATTTTAATGTTTT
TCCCCATGAACCACAGTTTTTATATTTCTACCAGAAAAGTAAAAA
TCTTT

In some embodiments, a donor template comprises a 5′ and/or 3′ homology arm homologous to a region of a G6PD locus. In some embodiments, a donor template comprises a 5′ and/or 3′ homology arm homologous to a region of a E2F4 locus. In some embodiments, a donor template comprises a 5′ and/or 3′ homology arm homologous to a region of a KIF11 locus.

### Exemplary Donor Template Sequences

exemplary donor template for insertion
at GAPDH locus
SEQ ID NO: 1198
GAAGACTGTGGATGGCCCCTCCGGGAAACTGTGGCGTGATGGCCG
CGGGGCTCTCCAGAACATCATCCCTGCCTCTACTGGCGCTGCCAA
GGCTGTGGGCAAGGTCA
TCCCTGAGCTGAACGGGAAGCTCACTGGCATGGCCTTCCGTGTCC
CCACTGCCAACGTGTCAGTGGTGGACCTGACCTGCCGTCTAGAAA
AACCTGCCAAATATGATGACATCAAGAAGGTGGTGAAGCAGGCGT
CGGAGGGCCCCCTCAAGGGCATCCTGGGCTACACTGAGCACCAGG
TGGTCTCCTCTGACTTCAACAGCGACACCCACTCCTCCACCTTTG
ACGCTGGGGCTGGCATTGCCCTCAACGACCACTTTGTCAAGCTCA
TTTCCTGGTATGTGGCTGGGGCCAGAGACTGGCTCTTAAAAAGTG
CAGGGTCTGGCGCCCTCTGGTGGCTGGCTCAGAAAAAGGGCCCTG
ACAACTCTTTACATCTTCTAGGTATGACAACGAGTTCGGATATAG
CAATAGAGTGGTCGATCTGATGGCTCATATGGCTAGCAAAGAGGG
AAGCGGAGCTACTAACTTCAGCCTGCTGAAGCAGGCTGGAGACGT
GGAGGAGAACCCTGGACCTATGGTGAGCAAGGGCGAGGAGCTGTT
CACCGGGGTGGTGCCCATCCTGGTCGAGCTGGACGGCGACGTAAA
CGGCCACAAGTTCAGCGTGTCCGGCGAGGGCGAGGGCGATGCCAC
CTACGGCAAGCTGACCCTGAAGTTCATCTGCACCACCGGCAAGCT
GCCCGTGCCCTGGCCCACCCTCGTGACCACCCTGACCTACGGCGT
GCAGTGCTTCAGCCGCTACCCCGACCACATGAAGCAGCACGACTT
CTTCAAGTCCGCCATGCCCGAAGGCTACGTCCAGGAGCGCACCAT
CTTCTTCAAGGACGACGGCAACTACAAGACCCGCGCCGAGGTGAA
GTTCGAGGGCGACACCCTGGTGAACCGCATCGAGCTGAAGGGCAT
CGACTTCAAGGAGGACGGCAACATCCTGGGGCACAAGCTGGAGTA
CAACTACAACAGCCACAACGTCTATATCATGGCCGACAAGCAGAA
GAACGGCATCAAGGTGAACTTCAAGATCCGCCACAACATCGAGGA
CGGCAGCGTGCAGCTCGCCGACCACTACCAGCAGAACACCCCCAT
CGGCGACGGCCCCGTGCTGCTGCCCGACAACCACTACCTGAGCAC
CCAGTCCGCCCTGAGCAAAGACCCCAACGAGAAGCGCGATCACAT
GGTCCTGCTGGAGTTCGTGACCGCCGCCGGGATCACTCTCGGCAT
GGACGAGCTGTACAAGTGAGCGGCCGCGTCGAGTCTAGAGGGCCC
GTTTAAACCCGCTGATCAGCCTCGACTGTGCCTTCTAGTTGCCAG
CCATCTGTTGTTTGCCCCTCCCCCGTGCCTTCCTTGACCCTGGAA
GGTGCCACTCCCACTGTCCTTTCCTAATAAAATGAGGAAATTGCA
TCGCATTGTCTGAGTAGGTGTCATTCTATTCTGGGGGGTGGGGTG
GGGCAGGACAGCAAGGGGGAGGATTGGGAAGACAATAGCAGGCAT
GCTGGGGATGCGGTGGGCTCTATGGATTTGGCTACAGCAACAGGG
TGGTGGACCTCATGGCCCACATGGCCTCCAAGGAGTAAGACCCCT
GGACCACCAGCCCCAGCAAGAGCACAAGAGGAAGAGAGAGACCCT
CACTGCTGGGGAGTCCCTGCCACACTCAGTCCCCCACCACACTGA
ATCTCCCCTCCTCACAGTTGCCATGTAGACCCCTTGAAGAGGGGA
GGGGCCTAGGGAGCCGCACCTTGTCATGTACCATCAATAAAGTAC
CCTGTGCTCAACCAGTTACTTGTCCTGTCTTATTCTAGGGTCTGG
GGCAGAGGGGAGGGAAGCTGGGCTTGTGTCAAGGTGAGACATTCT
TGCTGGGGAGGGACCTGGTATGTTCTCCTCAGACTGAGGGTAGGG
CCTCCAAACAGCCTTGCTTGCTTCGAGAACCATTTGCTTCCCGCT
CAGACGTCTTGAGTGCTACAGGAAGCTGGCACCACTACTTCAGAG
AACAAGGCCTTTTCCTCTCCTCGCTCCAGT
exemplary donor template for insertion
at TBP locus
SEQ ID NO: 1199
CTGACCACAGCTCTGCAAGCAGACTTCCATTTACAGTGAGGAGGT
GAGCATTGCATTGAACAAAAGATGGCGTTTTCACTTGGAATTAGT
TATCTGAAGCTTTAGGATTCCTCAGCAATATGATTATGAGACAAG
AAAGGAAGATTCAGAAATGAGTCTAGTTGAAGGCAGCAATTCAGA
GAAGAAGATTCAGTTGTTATCATTGCCGTCCTGCTTGGTTTATGG
CCTGGTTCAGGACCAAGGAGAGAAGTGTGAATACATGCCTCTTGA
GCTATAGAATGAGACGCTGGAGTCACTAAGATGATTTTTTAAAAG
TATTGTTTTATAAACAAAAATAAGATTGTGACAAGGGATTCCACT
ATTAATGTTTTCATGCCTGTGCCTTAATCTGACTGGGTATGGTGA
GAATTGTGCTTGCAGCTTTAAGGTAAGAATTTTACCATCTTAATA
TGTTAAGAAGTGCCATTTCAGTCTCTCATCTCTACTCCAACTTGT
CTTCTTAGGGGCTAAAGTGCGGGCCGAGATCTACGAGGCCTTCGA
GAATATCTACCCCATCCTGAAGGGCTTCAGAAAGACCACCGGAAG
CGGAGCTACTAACTTCAGCCTGCTGAAGCAGGCTGGAGACGTGGA
GGAGAACCCTGGACCTATGGTGAGCAAGGGCGAGGAGCTGTTCAC
CGGGGTGGTGCCCATCCTGGTCGAGCTGGACGGCGACGTAAACGG
CCACAAGTTCAGCGTGTCCGGCGAGGGCGAGGGCGATGCCACCTA
CGGCAAGCTGACCCTGAAGTTCATCTGCACCACCGGCAAGCTGCC
CGTGCCCTGGCCCACCCTCGTGACCACCCTGACCTACGGCGTGCA
GTGCTTCAGCCGCTACCCCGACCACATGAAGCAGCACGACTTCTT
CAAGTCCGCCATGCCCGAAGGCTACGTCCAGGAGCGCACCATCTT
CTTCAAGGACGACGGCAACTACAAGACCCGCGCCGAGGTGAAGTT
CGAGGGCGACACCCTGGTGAACCGCATCGAGCTGAAGGGCATCGA
CTTCAAGGAGGACGGCAACATCCTGGGGCACAAGCTGGAGTACAA
CTACAACAGCCACAACGTCTATATCATGGCCGACAAGCAGAAGAA
CGGCATCAAGGTGAACTTCAAGATCCGCCACAACATCGAGGACGG
CAGCGTGCAGCTCGCCGACCACTACCAGCAGAACACCCCCATCGG
CGACGGCCCCGTGCTGCTGCCCGACAACCACTACCTGAGCACCCA
GTCCGCCCTGAGCAAAGACCCCAACGAGAAGCGCGATCACATGGT
CCTGCTGGAGTTCGTGACCGCCGCCGGGATCACTCTCGGCATGGA
CGAGCTGTACAAGTGAGCGGCCGCGTCGAGTCTAGAGGGCCCGTT
TAAACCCGCTGATCAGCCTCGACTGTGCCTTCTAGTTGCCAGCCA
TCTGTTGTTTGCCCCTCCCCCGTGCCTTCCTTGACCCTGGAAGGT
GCCACTCCCACTGTCCTTTCCTAATAAAATGAGGAAATTGCATCG
CATTGTCTGAGTAGGTGTCATTCTATTCTGGGGGGGGGGTGGGGC
AGGACAGCAAGGGGGAGGATTGGGAAGACAATAGCAGGCATGCTG
GGGATGCGGTGGGCTCTATGGTAGGTGCTAAAGTCAGAGCAGAAA
TTTATGAAGCATTTGAAAACATCTACCCTATTCTAAAGGGATTCA
GGAAGACGACGTAATGGCTCTCATGTACCCTTGCCTCCCCCACCC
CCTTCTTTTTTTTTTTTTAAACAAATCAGTTTGTTTTGGTACCTT
TAAATGGTGGTGTTGTGAGAAGATGGATGTTGAGTTGCAGGGTGT
GGCACCAGGTGATGCCCTTCTGTAAGTGCCCACCGCGGGATGCCG
GGAAGGGGCATTATTTGTGCACTGAGAACACCGCGCAGCGTGACT
GTGAGTTGCTCATACCGTGCTGCTATCTGGGCAGCGCTGCCCATT
TATTTATATGTAGATTTTAAACACTGCTGTTGACAAGTTGGTTTG
AGGGAGAAAACTTTAAGTGTTAAAGCCACCTCTATAATTGATTGG
ACTTTTTAATTTTAATGTTTTTCCCCATGAACCACAGTTTTTATA
TTTCTACCAGAAAAGTAAAAATCTTT

### AAV Capsids

In some embodiments, the present disclosure provides one or more polynucleotide constructs (e.g., donor templates) packaged into an AAV capsid. In some embodiments, an AAV capsid is from or derived from an AAV capsid of an AAV2, 3, 4, 5, 6, 7, 8, 9, or 10 serotype or one or more hybrids thereof. In some embodiments, an AAV capsid is from an AAV ancestral serotype. In some embodiments, an AAV capsid is an ancestral (Anc) AAV capsid. An Anc capsid is created from a construct sequence that is constructed using evolutionary probabilities and evolutionary modeling to determine a probable ancestral sequence. In some embodiments, an AAV capsid has been modified in a manner known in the art (see e.g., Büning and Srivastava, Capsid modifications for targeting and improving the efficacy of AAV vectors, Mol Ther Methods Clin Dev. 2019)

In some embodiments, as provided herein, any combination of AAV capsids and AAV constructs (e.g., comprising AAV ITRs) may be used in recombinant AAV (rAAV) particles of the present disclosure. In some embodiments, an AAV ITR is from or derived from an AAV ITR of AAV2, 3, 4, 5, 6, 7, 8, 9, or 10. For example, wild-type or variant AA6 ITRs and AAV6 capsid, wild-type or variant AAV2 ITRs and AAV6 capsid, etc. In some embodiments of the present disclosure, an AAV particle is wholly comprised of AAV6 components (e.g., capsid and ITRs are AAV6 serotype). In some embodiments, an AAV particle is an AAV6/2, AAV6/8 or AAV6/9 particle (e.g., an AAV2, AAV8 or AAV9 capsid with an AAV construct having AAV6 ITRs).

### Generation of iNK Cells

In some embodiments, the present disclosure provides methods of generating iNK cells (e.g., genetically modified iNK cells) that are derived from stem cells described herein.

In some embodiments, genetic modifications (e.g., genomic edits) present in an iNK cell of the present disclosure can be made at any stage during the reprogramming process from donor cell to iPSC, during the iPSC stage, and/or at any stage of the process of differentiating the iPSC to an iNK state, e.g., at an intermediary state, such as, for example, an iPSC-derived HSC state, or even up to or at the final iNK cell state.

For example, one or more genomic edits present in an edited iNK cell of the present disclosure may be made at one or more different cell stages (e.g., reprogramming from donor to iPSC, differentiation of iPSC to iNK). In some embodiments, one or more genomic edits present in modified genetically modified iNK cell provided herein is made before reprogramming a donor cell to an iPSC state. In some embodiments, all edits present in a genetically modified iNK cell provided herein are made at the same time, in close temporal proximity, and/or at the same cell stage of the reprogramming/differentiation process, e.g., at the donor cell stage, during the reprogramming process, at the iPSC stage, or during the differentiation process, e.g., from iPSC to iNK. In some embodiments, two or more edits present in a genetically modified iNK cell provided herein are made at different times and/or at different cell stages of the reprogramming/differentiation process from donor cell to iPSC to iNK. For example, in some embodiments, a first edit is made at the donor cell stage and a second (different) edit is made at the iPSC stage. In some embodiments, a first edit is made at the reprogramming stage (e.g., donor to iPSC) and a second (different) edit is made at the iPSC stage.

A variety of cell types can be used as a donor cell that can be subjected to reprogramming, differentiation, and/or genomic editing strategies described herein. For example, the donor cell can be a pluripotent stem cell or a differentiated cell, e.g., a somatic cell, such as, for example, a fibroblast or a T lymphocyte. In some embodiments, donor cells are manipulated (e.g., subjected to reprogramming, differentiation, and/or genomic editing) to generate iNK cells described herein.

A donor cell can be from any suitable organism. For example, in some embodiments, the donor cell is a mammalian cell, e.g., a human cell or a non-human primate cell. In some embodiments, the donor cell is a somatic cell. In some embodiments, the donor cell is a stem cell or progenitor cell. In certain embodiments, the donor cell is not or was not part of a human embryo and its derivation does not involve destruction of a human embryo.

In some embodiments, an edited iNK cell is derived from an iPSC, which in turn is derived from a somatic donor cell. Any suitable somatic cell can be used in the generation of iPSCs, and in turn, the generation of iNK cells. Suitable strategies for deriving iPSCs from various somatic donor cell types have been described and are known in the art. In some embodiments, a somatic donor cell is a fibroblast cell. In some embodiments, a somatic donor cell is a mature T cell.

For example, in some embodiments, a somatic donor cell, from which an iPSC, and subsequently an iNK cell is derived, is a developmentally mature T cell (a T cell that has undergone thymic selection). One hallmark of developmentally mature T cells is a rearranged T cell receptor locus. During T cell maturation, the TCR locus undergoes V(D)J rearrangements to generate complete V-domain exons. These rearrangements are retained throughout reprogramming of a T cells to an iPSC, and throughout differentiation of the resulting iPSC to a somatic cell.

In certain embodiments, a somatic donor cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+naïve T cell, a TH17 CD4⁺ T cell, a TH1 CD4⁺ T cell, a TH2 CD4⁺ T cell, a TH9 CD4⁺ T cell, a CD4+Foxp3⁺ T cell, a CD4⁺ CD25⁺ CD127 T cell, or a CD4⁺ CD25⁺ CD127 Foxp3⁺ T cell.

T cells can be advantageous for the generation of iPSCs. For example, T cells can be edited with relative case, e.g., by CRISPR-based methods or other gene-editing methods. Additionally, the rearranged TCR locus allows for genetic tracking of individual cells and their daughter cells. For example, if the reprogramming, expansion, culture, and/or differentiation strategies involved in the generation of NK cells a clonal expansion of a single cell, the rearranged TCR locus can be used as a genetic marker unambiguously identifying a cell and its daughter cells. This, in turn, allows for the characterization of a cell population as truly clonal, or for the identification of mixed populations, or contaminating cells in a clonal population. Another potential advantage of using T cells in generating iNK cells carrying multiple edits is that certain karyotypic aberrations associated with chromosomal translocations are selected against in T cell culture. Such aberrations can pose a concern when editing cells by CRISPR technology, and in particular when generating cells carrying multiple edits. Using T cell derived iPSCs as a starting point for the derivation of therapeutic lymphocytes can allow for the expression of a pre-screened TCR in the lymphocytes, e.g., via selecting the T cells for binding activity against a specific antigen, e.g., a tumor antigen, reprogramming the selected T cells to iPSCs, and then deriving lymphocytes from these iPSCs that express the TCR (e.g., T cells). This strategy can allow for activating the TCR in other cell types, e.g., by genetic or epigenetic strategies. Additionally, T cells retain at least part of their “epigenetic memory” throughout the reprogramming process, and thus subsequent differentiation of the same or a closely related cell type, such as iNK cells can be more efficient and/or result in higher quality cell populations as compared to approaches using non-related cells, such as fibroblasts, as a starting point for iNK derivation.

In some embodiments, a donor cell being manipulated, e.g., a cell being reprogrammed and/or undergoing genomic editing, is one or more of a long-term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a fibroblast, a monocyte-derived macrophage or dendritic cell, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte.

In some embodiments, a donor cell is one or more of a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In some embodiments, a donor cell is one or more of a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In some embodiments, a donor cell is one or more of a myeloid progenitor cell (e.g., a common myeloid progenitor (CMP) cell or granulocyte macrophage progenitor (GMP) cell). In some embodiments, a donor cell is one or more of a lymphoid progenitor cell, e.g., a common lymphoid progenitor (CLP) cell. In some embodiments, a donor cell is one or more of an erythroid progenitor cell (e.g., an MEP cell). In some embodiments, a donor cell is one or more of a hematopoietic stem/progenitor cell (e.g., a long term HSC (LT-HSC), short term HSC (ST-HSC), MPP cell, or lineage restricted progenitor (LRP) cell). In certain embodiments, the donor cell is a CD34⁺ cell, CD34⁺CD90⁺ cell, CD34⁺CD38-cell, CD34⁺CD90⁺CD49f⁺CD38⁻CD45RA cell, CD105⁺ cell, CD31⁺, or CD133⁺ cell, or a CD34⁺CD90⁺ CD133⁺ cell. In some embodiments, a donor cell is one or more of an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In some embodiments, a donor cell is one or more of a mobilized peripheral blood hematopoietic CD34⁺ cell (after the patient is treated with a mobilization agent, e.g., G-CSF or Plerixafor). In some embodiments, a donor cell is a peripheral blood endothelial cell. In some embodiments, a donor cell is a peripheral blood natural killer cell.

In some embodiments, a donor cell is a dividing cell. In some embodiments, a donor cell is a non-dividing cell.

In some embodiments, a genetically modified (e.g., edited) iNK cell resulting from one or more methods and/or strategies described herein, are administered to a subject in need thereof, e.g., in the context of an immuno-oncology therapeutic approach. In some embodiments, donor cells, or any cells of any stage of the reprogramming, differentiating, and/or editing strategies provided herein, can be maintained in culture or stored (e.g., frozen in liquid nitrogen) using any suitable method known in the art, e.g., for subsequent characterization or administration to a subject in need thereof.

### Genome Editing Systems

Genome editing systems of the present disclosure may be used, for example, to edit stem cells. In some embodiments, genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (“Makarova”)), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e., target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder”) describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”) describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS. 111(10):E924-932, Mar. 11, 2014 (“Davis”) (describing Alt-HDR); Frit et al. DNA Repair 17(2014) 81-97 (“Frit”) (describing Alt-NHEJ); and Iyama and Wilson III, DNA Repair (Amst.) 2013-August; 12(8): 620-636 (“Iyama”) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a target gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”). Alternatively, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNA (gRNA) Molecules

Guide RNAs (gRNAs) of the present disclosure may be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (“Briner”)), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek 2012”)).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”)), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (“Nishimasu 2014”) and Nishimasu et al., Cell 162, 1113-1126 Aug. 27, 2015 (“Nishimasu 2015”)). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop near the 3′ portion of the second complementarity domain is referred to variously as the “proximal domain.” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a RNA-guided nuclease that does not require a tracrRNA to function. (Zetsche et al., 2015, Cell 163, 759-771 Oct. 22, 2015 (“Zetsche I”)). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

gRNA Design

Methods for selection and validation of target sequences as well as off-target analyses have been described previously, e.g., in Mali; Hsu; Fu et al., (2014) Nat Biotechnol 32(3): 279-84, Heigwer et al., (2014) Nat methods 11(2):122-3; Bac et al. (2014) Bioinformatics 30(10): 1473-5; and Xiao A et al. (2014) Bioinformatics 30(8): 1180-1182. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

For example, methods for selection and validation of target sequences as well as off-target analyses can be performed using cas-offinder (Bae S, Park J, Kim J-S. Cas-OFFinder: a fast and versatile algorithm that searches for potential off-target sites of Cas9 RNA-guided endonucleases. Bioinformatics. 2014; 30:1473-5). Cas-offinder is a tool that can quickly identify all sequences in a genome that have up to a specified number of mismatches to a guide sequence.

As another example, methods for scoring how likely a given sequence is to be an off-target (e.g., once candidate target sequences are identified) can be performed. An exemplary score includes a Cutting Frequency Determination (CFD) score, as described by Doench J G, Fusi N, Sullender M, Hegde M, Vaimberg E W, Donovan K F, et al. Optimized sgRNA design to maximize activity and minimize off-target effects of CRISPR-Cas9. Nat Biotechnol. 2016; 34:184-91.

gRNA Modifications

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases. In certain embodiments, a DNA extension may comprise a sequence set forth in Table 3.

Exemplary suitable 5′ extensions for Cpf1 guide RNAs are provided in Table 3 below:

TABLE 3
Exemplary Cpf1 gRNA 5′ Extensions

| SEQ ID | | 5′ |
|---|---|---|
| NO: | 5′ extension sequence | modification |
| 1 | rCrUrUrUrU | +5 RNA |
| 2 | rArArGrArCrCrUrUrUrU | +10 RNA |
| 3 | rArUrGrUrGrUrUrUrUrUrGrUr | +25 RNA |
| | CrArArArArGrArCrCrUrUrUrU | |
| 4 | rArGrGrCrCrArGrCrUrUrGrCr | +60 RNA |
| | CrGrGrUrUrUrUrUrUrArGrUrC | |
| | rGrUrGrCrUrGrCrUrUrCrArUr | |
| | GrUrGrUrUrUrUrUrGrUrCrArA | |
| | rArArGrArCrCrUrUrUrU | |
| 5 | CTTTT | +5 DNA |
| 6 | AAGACCTTTT | +10 DNA |
| 7 | ATGTGTTTTTGTCAAAAGACCTTTT | +25 DNA |
| 8 | AGGCCAGCTTGCCGGTTTTTTAGTC | +60 DNA |
| | GTGCTGCTTCATGTGTTTTTGTCAA | |
| | AAGACCTTTT | |
| 9 | TTTTTGTCAAAAGACCTTTT | +20 DNA |
| 10 | GCTTCATGTGTTTTTGTCAAAAGAC | +30 DNA |
| | CTTTT | |
| 11 | GCCGGTTTTTTAGTCGTGCTGCTTC | +50 DNA |
| | ATGTGTTTTTGTCAAAAGACCTTTT | |
| 12 | TAGTCGTGCTGCTTCATGTGTTTTTG | +40 DNA |
| | TCAAAAGACCTTTT | |
| 13 | C*C*GAAGTTTTCTTCGGTTTT | +20 DNA + |
| | | 2×PS |
| 14 | T*T*TTTCCGAAGTTTTCTTCGGT | +25 DNA + |
| | TTT | 2×PS |
| 15 | A*A*CGCTTTTTCCGAAGTTTTCT | +30 DNA + |
| | TCGGTTTT | 2×PS |
| 16 | G*C*GTTGTTTTCAACGCTTTTTC | +41 DNA + |
| | CGAAGTTTTCTTCGGTTTT | 2×PS |
| 17 | G*G*CTTCTTTTGAAGCCTTTTTG | +62 DNA + |
| | CGTTGTTTTCAACGCTTTTTCCGA | 2xPS |
| | AGTTTTCTTCGGTTTT | |
| 18 | A*T*GTGTTTTTGTCAAAAGACCTTTT | +25 DNA + |
| | | 2×PS |
| 19 | AAAAAAAAAAAAAAAAAAAAAAAAA | +25 A |
| 20 | TTTTTTTTTTTTTTTTTTTTTTTTT | +25 T |
| 21 | mA*mU*rGrUrGrUrUrUrUrUrGrUr | +25 RNA + |
| | CrArArArArGrArCrCrUrUrUrU | 2×PS |
| 22 | mA*mA*rArArArArArArArArArAr | PolyA RNA + |
| | ArArArArArArArArArArArArA | 2×PS |
| 23 | mU*mU*rUrUrUrUrUrUrUrUrUrUr | PolyU RNA + |
| | UrUrUrUrUrUrUrUrUrUrUrUrU | 2×PS |

All bases are in upper case
Lowercase “r” represents RNA, 2′-hydroxy; bases not modified by an “r” are DNA
All bases are linked via standard phosphodiester bonds except as noted:
“r” represents phosphorothioate modification
“PS” represents phosphorothioate modification

In certain embodiments, a gRNA used herein includes a DNA extension as well as a chemical modification, e.g., one or more phosphorothioate linkage modifications, one or more phosphorodithioate (PS2) linkage modifications, one or more 2′-O-methyl modifications, or one or more additional suitable chemical gRNA modification disclosed herein, or combinations thereof. In certain embodiments, the one or more modifications may be at the 5′ end of the gRNA, at the 3′ end of the gRNA, or combinations thereof.

Without wishing to be bound by theory, it is contemplated that any DNA extension may be used with any gRNA disclosed herein, so long as it does not hybridize to the target nucleic acid being targeted by the gRNA and it also exhibits an increase in editing at the target nucleic acid site relative to a gRNA which does not include such a DNA extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA. 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, a gRNA used herein includes an RNA extension as well as one or more phosphorothioate linkage modifications, one or more phosphorodithioate (PS2) linkage modifications, one or more 2′-O-methyl modifications, one or more additional suitable gRNA modification, e.g., chemical modification, disclosed herein, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth herein.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end and/or a chemical modification as disclosed herein, is complexed with a RNA-guided nuclease, e.g., an AsCpf1 nuclease, to form an RNP, which is then employed to edit a target cell, e.g., a pluripotent stem cell or a daughter cell thereof.

Additional suitable gRNA modifications will be apparent to those of ordinary skill in the art based on the present disclosure. Suitable gRNA modifications include, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

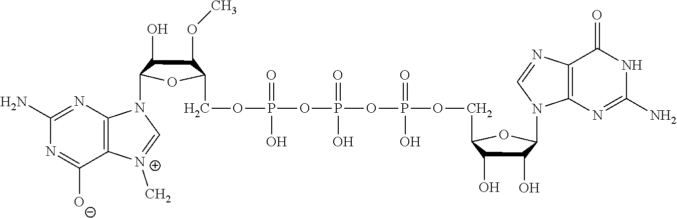

The cap or cap analog can be included during either chemical or enzymatic synthesis of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical or enzymatic synthesis, using a polyadenosine polymerase (e.g., E. coli Poly(A)Polymerase).

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

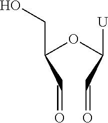

wherein “U” can be an unmodified or modified uridine.

The 3′ terminal U ribose can be modified with a 2′3′ cyclic phosphate as shown below:

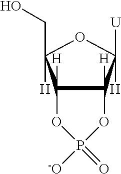

wherein “U” can be an unmodified or modified uridine.

Guide RNAs can contain 3′ nucleotides that can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into a gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂, alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including. e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, including without limitation methylene, propylene, ether, or amino bridges; O-amino (wherein amino can be, e.g., NH₂, alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂, alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into a gRNA. In certain embodiments, O- and N-alkylated nucleotides, e.g., N6-methyl adenosine, can be incorporated into a gRNA. In certain embodiments, one or more or all of the nucleotides in a gRNA are deoxynucleotides.

Guide RNAs can also include one or more cross-links between complementary regions of the crRNA (at its 3′ end) and the tracrRNA (at its 5′ end) (e.g., within a “tetraloop” structure and/or positioned in any stem loop structure occurring within a gRNA). A variety of linkers are suitable for use. For example, guide RNAs can include common linking moieties including, without limitation, polyvinylether, polyethylene, polypropylene, polyethylene glycol (PEG), polyvinyl alcohol (PVA), polyglycolide (PGA), polylactide (PLA), polycaprolactone (PCL), and copolymers thereof.

In some embodiments, a bifunctional cross-linker is used to link a 5′ end of a first gRNA fragment and a 3′ end of a second gRNA fragment, and the 3′ or 5′ ends of the gRNA fragments to be linked are modified with functional groups that react with the reactive groups of the cross-linker. In general, these modifications comprise one or more of amine, sulfhydryl, carboxyl, hydroxyl, alkene (e.g., a terminal alkene), azide and/or another suitable functional group. Multifunctional (e.g. bifunctional) cross-linkers are also generally known in the art, and may be either heterofunctional or homofunctional, and may include any suitable functional group, including without limitation isothiocyanate, isocyanate, acyl azide, an NHS ester, sulfonyl chloride, tosyl ester, tresyl ester, aldehyde, amine, epoxide, carbonate (e.g., Bis(p-nitrophenyl) carbonate), aryl halide, alkyl halide, imido ester, carboxylate, alkyl phosphate, anhydride, fluorophenyl ester, HOBt ester, hydroxymethyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine, TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc. In some embodiments, a first gRNA fragment comprises a first reactive group and the second gRNA fragment comprises a second reactive group. For example, the first and second reactive groups can each comprise an amine moiety, which are crosslinked with a carbonate-containing bifunctional crosslinking reagent to form a urea linkage. In other instances, (a) the first reactive group comprises a bromoacetyl moiety and the second reactive group comprises a sulfhydryl moiety, or (b) the first reactive group comprises a sulfhydryl moiety and the second reactive group comprises a bromoacetyl moiety, which are crosslinked by reacting the bromoacetyl moiety with the sulfhydryl moiety to form a bromoacetyl-thiol linkage. These and other cross-linking chemistries are known in the art, and are summarized in the literature, including by Greg T. Hermanson, Bioconjugate Techniques, 3rd Ed. 2013, published by Academic Press.

Exemplary gRNAs

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cpf-1 nuclease, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and thus contain uracil instead of thymidine nucleotides. For example, a guide RNA comprising a targeting domain sequence consisting of RNA nucleotides, and described by the DNA sequence TCTGCAGAAATGTTCCCCGT (SEQ ID NO: 24) would have a targeting domain of the corresponding RNA sequence UCUGCAGAAAUGUUCCCCGU (SEQ ID NO: 25). As will be apparent to the skilled artisan, such a targeting sequence would be linked to a suitable guide RNA scaffold, e.g., a crRNA scaffold sequence or a chimeric crRNA/tracrRNA scaffold sequence. Suitable gRNA scaffold sequences are known to those of ordinary skill in the art. For AsCpf1, for example, a suitable scaffold sequence comprises the sequence UAAUUUCUACUCUUGUAGAU (SEQ ID NO: 26) added to the 5′-terminus of the targeting domain. In the example above, this would result in a Cpf1 guide RNA of the sequence UAAUUUCUACUCUUGUAGAUUCUGCAGAAAUGUUCCCCGU (SEQ ID NO: 27). Those of skill in the art would further understand how to modify such a guide RNA, e.g., by adding a DNA extension (e.g., in the example above, adding a 25-mer DNA extension as described herein would result, for example, in a guide RNA of the sequence ATGTGTTTTTGTCAAAAGACCTTTTrUrArArUrUrUrCrUrArCrUrCrUrUrGrUrArGrArU rUrCrUrGrCrArGrArArArUrGrUrUrCrCrCrCrGrU) (SEQ ID NO: 28). It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

In some embodiments the gRNA for use in the disclosure is a gRNA targeting TGFβRII (TGFβRII gRNA). In some embodiments, the gRNA targeting TGFβRII is one or more of the gRNAs described in Table 4.

TABLE 4
Exemplary TGFβRII gRNAs

| | gRNA Targeting | | | SEQ |
|---|---|---|---|---|
| | Domain Sequence | | | ID |
| Name | (DNA) | Length | Enzyme | NO: |
| TGFBR24326 | CAGGACGATGTGCAGCGGCC | 20 | AsCpf1 RR | 29 |
| TGFBR24327 | ACCGCACGTTCAGAAGTCGG | 20 | AsCpf1 RR | 30 |
| TGFBR24328 | ACAACTGTGTAAATTTTGTG | 20 | AsCpf1 RR | 31 |
| TGFBR24329 | CAACTGTGTAAATTTTGTGA | 20 | AsCpf1 RR | 32 |
| TGFBR24330 | ACCTGTGACAACCAGAAATC | 20 | AsCpf1 RR | 33 |
| TGFBR24331 | CCTGTGACAACCAGAAATCC | 20 | AsCpf1 RR | 34 |
| TGFBR24332 | TGTGGCTTCTCACAGATGGA | 20 | AsCpf1 RR | 35 |
| TGFBR24333 | TCTGTGAGAAGCCACAGGAA | 20 | AsCpf1 RR | 36 |
| TGFBR24334 | AAGCTCCCCTACCATGACTT | 20 | AsCpf1 RR | 37 |
| TGFBR24335 | GAATAAAGTCATGGTAGGGG | 20 | AsCpf1 RR | 38 |
| TGFBR24336 | AGAATAAAGTCATGGTAGGG | 20 | AsCpf1 RR | 39 |
| TGFBR24337 | CTACCATGACTTTATTCTGG | 20 | AsCpf1 RR | 40 |
| TGFBR24338 | TACCATGACTTTATTCTGGA | 20 | AsCpf1 RR | 41 |
| TGFBR24339 | TAATGCACTTTGGAGAAGCA | 20 | AsCpf1 RR | 42 |
| TGFBR24340 | TTCATAATGCACTTTGGAGA | 20 | AsCpf1 RR | 43 |
| TGFBR24341 | AAGTGCATTATGAAGGAAAA | 20 | AsCpf1 RR | 44 |
| TGFBR24342 | TGTGTTCCTGTAGCTCTGAT | 20 | AsCpf1 RR | 45 |
| TGFBR24343 | TGTAGCTCTGATGAGTGCAA | 20 | AsCpf1 RR | 46 |
| TGFBR24344 | AGTGACAGGCATCAGCCTCC | 20 | AsCpf1 RR | 47 |
| TGFBR24345 | AGTGGTGGCAGGAGGCTGAT | 20 | AsCpf1 RR | 48 |
| TGFBR24346 | AGGTTGAACTCAGCTTCTGC | 20 | AsCpf1 RR | 49 |
| TGFBR24347 | CAGGTTGAACTCAGCTTCTG | 20 | AsCpf1 RR | 50 |
| TGFBR24348 | ACCTGGGAAACCGGCAAGAC | 20 | AsCpf1 RR | 51 |
| TGFBR24349 | CGTCTTGCCGGTTTCCCAGG | 20 | AsCpf1 RR | 52 |
| TGFBR24350 | GCGTCTTGCCGGTTTCCCAG | 20 | AsCpf1 RR | 53 |
| TGFBR24351 | TGAGCTTCCGCGTCTTGCCG | 20 | AsCpf1 RR | 54 |
| TGFBR24352 | GCGAGCACTGTGCCATCATC | 20 | AsCpf1 RR | 55 |
| TGFBR24353 | GGATGATGGCACAGTGCTCG | 20 | AsCpf1 RR | 56 |
| TGFBR24354 | AGGATGATGGCACAGTGCTC | 20 | AsCpf1 RR | 57 |
| TGFBR24355 | CGTGTGCCAACAACATCAAC | 20 | AsCpf1 RR | 58 |
| TGFBR24356 | GCTCAATGGGCAGCAGCTCT | 20 | AsCpf1 RR | 59 |
| TGFBR24357 | ACCAGGGTGTCCAGCTCAAT | 20 | AsCpf1 RR | 60 |
| TGFBR24358 | CACCAGGGTGTCCAGCTCAA | 20 | AsCpf1 RR | 61 |
| TGFBR24359 | CCACCAGGGTGTCCAGCTCA | 20 | AsCpf1 RR | 62 |
| TGFBR24360 | GCTTGGCCTTATAGACCTCA | 20 | AsCpf1 RR | 63 |
| TGFBR24361 | GAGCAGTTTGAGACAGTGGC | 20 | AsCpf1 RR | 64 |
| TGFBR24362 | AGAGGCATACTCCTCATAGG | 20 | AsCpf1 RR | 65 |
| TGFBR24363 | CTATGAGGAGTATGCCTCTT | 20 | AsCpf1 RR | 66 |
| TGFBR24364 | AAGAGGCATACTCCTCATAG | 20 | AsCpf1 RR | 67 |
| TGFBR24365 | TATGAGGAGTATGCCTCTTG | 20 | AsCpf1 RR | 68 |
| TGFBR24366 | GATTGATGTCTGAGAAGATG | 20 | AsCpf1 RR | 69 |
| TGFBR24367 | CTCCTCAGCCGTCAGGAACT | 20 | AsCpf1 RR | 70 |
| TGFBR24368 | GTTCCTGACGGCTGAGGAGC | 20 | AsCpf1 RR | 71 |
| TGFBR24369 | GCTCCTCAGCCGTCAGGAAC | 20 | AsCpf1 RR | 72 |
| TGFBR24370 | TGACGGCTGAGGAGCGGAAG | 20 | AsCpf1 RR | 73 |
| TGFBR24371 | TCTTCCGCTCCTCAGCCGTC | 20 | AsCpf1 RR | 74 |
| TGFBR24372 | AACTCCGTCTTCCGCTCCTC | 20 | AsCpf1 RR | 75 |
| TGFBR24373 | CAACTCCGTCTTCCGCTCCT | 20 | AsCpf1 RR | 76 |
| TGFBR24374 | CCAACTCCGTCTTCCGCTCC | 20 | AsCpf1 RR | 77 |
| TGFBR24375 | ACGCCAAGGGCAACCTACAG | 20 | AsCpf1 RR | 78 |
| TGFBR24376 | CGCCAAGGGCAACCTACAGG | 20 | AsCpf1 RR | 79 |
| TGFBR24377 | AGCTGATGACATGCCGCGTC | 20 | AsCpf1 RR | 80 |
| TGFBR24378 | GGGCGAGGGAGCTGCCCAGC | 20 | AsCpf1 RR | 81 |
| TGFBR24379 | CGGGCGAGGGAGCTGCCCAG | 20 | AsCpf1 RR | 82 |
| TGFBR24380 | CCGGGCGAGGGAGCTGCCCA | 20 | AsCpf1 RR | 83 |
| TGFBR24381 | TCGCCCGGGGGATTGCTCAC | 20 | AsCpf1 RR | 84 |
| TGFBR24382 | ACATGGAGTGTGATCACTGT | 20 | AsCpf1 RR | 85 |
| TGFBR24383 | CAGTGATCACACTCCATGTG | 20 | AsCpf1 RR | 86 |
| TGFBR24384 | TGTGGGAGGCCCAAGATGCC | 20 | AsCpf1 RR | 87 |
| TGFBR24385 | TGTGCACGATGGGCATCTTG | 20 | AsCpf1 RR | 88 |
| TGFBR24386 | CGAGGATATTGGAGCTCTTG | 20 | AsCpf1 RR | 89 |
| TGFBR24387 | ATATCCTCGTGAAGAACGAC | 20 | AsCpf1 RR | 90 |
| TGFBR24388 | GACGCAGGGAAAGCCCAAAG | 20 | AsCpf1 RR | 91 |
| TGFBR24389 | CTGCGTCTGGACCCTACTCT | 20 | AsCpf1 RR | 92 |
| TGFBR24390 | TGCGTCTGGACCCTACTCTG | 20 | AsCpf1 RR | 93 |
| TGFBR24391 | CAGACAGAGTAGGGTCCAGA | 20 | AsCpf1 RR | 94 |
| TGFBR24392 | GCCAGCACGATCCCACCGCA | 20 | AsCpf1 RVR | 95 |
| TGFBR24393 | AAGGAAAAAAAAAAGCCTGG | 20 | AsCpf1 RVR | 96 |
| TGFBR24394 | ACACCAGCAATCCTGACTTG | 20 | AsCpf1 RVR | 97 |
| TGFBR24395 | ACTAGCAACAAGTCAGGATT | 20 | AsCpf1 RVR | 98 |
| TGFBR24396 | GCAACTCCCAGTGGTGGCAG | 20 | AsCpf1 RVR | 99 |
| TGFBR24397 | TGTCATCATCATCTTCTACT | 20 | AsCpf1 RVR | 100 |
| TGFBR24398 | GACCTCAGCAAAGCGACCTT | 20 | AsCpf1 RVR | 101 |
| TGFBR24399 | AGGCCAAGCTGAAGCAGAAC | 20 | AsCpf1 RVR | 102 |
| TGFBR24400 | AGGAGTATGCCTCTTGGAAG | 20 | AsCpf1 RVR | 103 |
| TGFBR24401 | CCTCTTGGAAGACAGAGAAG | 20 | AsCpf1 RVR | 104 |
| TGFBR24402 | TTCTCATGCTTCAGATTGAT | 20 | AsCpf1 RVR | 105 |
| TGFBR24403 | CTCGTGAAGAACGACCTAAC | 20 | AsCpf1 RVR | 106 |
| TGFbR2036 | GGCCGCTGCACATCGTCCTG | 20 | SpyCas9 | 107 |
| TGFbR2037 | GCGGGGTCTGCCATGGGTCG | 20 | SpyCas9 | 108 |
| TGFbR2038 | AGTTGCTCATGCAGGATTTC | 20 | SpyCas9 | 109 |
| TGFbR2039 | CCAGAATAAAGTCATGGTAG | 20 | SpyCas9 | 110 |
| TGFbR2040 | CCCCTACCATGACTTTATTC | 20 | SpyCas9 | 111 |
| TGFbR2041 | AAGTCATGGTAGGGGAGCTT | 20 | SpyCas9 | 112 |
| TGFbR2042 | AGTCATGGTAGGGGAGCTTG | 20 | SpyCas9 | 113 |
| TGFbR2043 | ATTGCACTCATCAGAGCTAC | 20 | SpyCas9 | 114 |
| TGFbR2044 | CCTAGAGTGAAGAGATTCAT | 20 | SpyCas9 | 115 |
| TGFbR2045 | CCAATGAATCTCTTCACTCT | 20 | SpyCas9 | 116 |
| TGFbR2046 | AAAGTCATGGTAGGGGAGCT | 20 | SpyCas9 | 117 |
| TGFbR2047 | GTGAGCAATCCCCCGGGCGA | 20 | SpyCas9 | 118 |
| TGFbR2048 | GTCGTTCTTCACGAGGATAT | 20 | SpyCas9 | 119 |
| TGFbR2049 | GCCGCGTCAGGTACTCCTGT | 20 | SpyCas9 | 120 |
| TGFbR2050 | GACGCGGCATGTCATCAGCT | 20 | SpyCas9 | 121 |
| TGFbR2051 | GCTTCTGCTGCCGGTTAACG | 20 | SpyCas9 | 122 |
| TGFbR2052 | GTGGATGACCTGGCTAACAG | 20 | SpyCas9 | 123 |
| TGFbR2053 | GTGATCACACTCCATGTGGG | 20 | SpyCas9 | 124 |
| TGFbR2054 | GCCCATTGAGCTGGACACCC | 20 | SpyCas9 | 125 |
| TGFbR2055 | GCGGTCATCTTCCAGGATGA | 20 | SpyCas9 | 126 |
| TGFbR2056 | GGGAGCTGCCCAGCTTGCGC | 20 | SpyCas9 | 127 |
| TGFbR2057 | GTTGATGTTGTTGGCACACG | 20 | SpyCas9 | 128 |
| TGFbR2058 | GGCATCTTGGGCCTCCCACA | 20 | SpyCas9 | 129 |
| TGFbR2059 | GCGGCATGTCATCAGCTGGG | 20 | SpyCas9 | 130 |
| TGFbR2060 | GCTCCTCAGCCGTCAGGAAC | 20 | SpyCas9 | 131 |
| TGFbR2061 | GCTGGTGTTATATTCTGATG | 20 | SpyCas9 | 132 |
| TGFbR2062 | CCGACTTCTGAACGTGCGGT | 20 | SpyCas9 | 133 |
| TGFbR2063 | TGCTGGCGATACGCGTCCAC | 20 | SpyCas9 | 134 |
| TGFbR2064 | CCCGACTTCTGAACGTGCGG | 20 | SpyCas9 | 135 |
| TGFbR2065 | CCACCGCACGTTCAGAAGTC | 20 | SpyCas9 | 136 |
| TGFbR2066 | TCACCCGACTTCTGAACGTG | 20 | SpyCas9 | 137 |
| TGFbR2067 | CCCACCGCACGTTCAGAAGT | 20 | SpyCas9 | 138 |
| TGFbR2068 | CGAGCAGCGGGGTCTGCCAT | 20 | SpyCas9 | 139 |
| TGFbR2069 | ACGAGCAGCGGGGTCTGCCA | 20 | SpyCas9 | 140 |
| TGFbR2070 | AGCGGGGTCTGCCATGGGTC | 20 | SpyCas9 | 141 |
| TGFbR2071 | CCTGAGCAGCCCCCGACCCA | 20 | SpyCas9 | 142 |
| TGFbR2072 | CCATGGGTCGGGGGCTGCTC | 20 | SpyCas9 | 143 |
| TGFbR2073 | AACGTGCGGTGGGATCGTGC | 20 | SpyCas9 | 144 |
| TGFbR2074 | GGACGATGTGCAGCGGCCAC | 20 | SpyCas9 | 145 |
| TGFbR2075 | GTCCACAGGACGATGTGCAG | 20 | SpyCas9 | 146 |
| TGFbR2076 | CATGGGTCGGGGGCTGCTCA | 20 | SpyCas9 | 147 |
| TGFbR2077 | CAGCGGGGTCTGCCATGGGT | 20 | SpyCas9 | 148 |
| TGFbR2078 | ATGGGTCGGGGGCTGCTCAG | 20 | SpyCas9 | 149 |
| TGFbR2079 | CGGGGTCTGCCATGGGTCGG | 20 | SpyCas9 | 150 |
| TGFbR2080 | AGGAAGTCTGTGTGGCTGTA | 20 | SpyCas9 | 151 |
| TGFbR2081 | CTCCATCTGTGAGAAGCCAC | 20 | SpyCas9 | 152 |
| TGFbR2082 | ATGATAGTCACTGACAACAA | 20 | SpyCas9 | 153 |
| TGFbR2083 | GATGCTGCAGTTGCTCATGC | 20 | SpyCas9 | 154 |
| TGFbR2084 | ACAGCCACACAGACTTCCTG | 20 | SpyCas9 | 155 |
| TGFbR2085 | GAAGCCACAGGAAGTCTGTG | 20 | SpyCas9 | 156 |
| TGFbR2086 | TTCCTGTGGCTTCTCACAGA | 20 | SpyCas9 | 157 |
| TGFbR2087 | CTGTGGCTTCTCACAGATGG | 20 | SpyCas9 | 158 |
| TGFbR2088 | TCACAAAATTTACACAGTTG | 20 | SpyCas9 | 159 |
| TGFbR2089 | GACAACATCATCTTCTCAGA | 20 | SpyCas9 | 160 |
| TGFbR2090 | TCCAGAATAAAGTCATGGTA | 20 | SpyCas9 | 161 |
| TGFbR2091 | GGTAGGGGAGCTTGGGGTCA | 20 | SpyCas9 | 162 |
| TGFbR2092 | TTCTCCAAAGTGCATTATGA | 20 | SpyCas9 | 163 |
| TGFbR2093 | CATCTTCCAGAATAAAGTCA | 20 | SpyCas9 | 164 |
| TGFbR2094 | CACATGAAGAAAGTCTCACC | 20 | SpyCas9 | 165 |
| TGFbR2095 | TTCCAGAATAAAGTCATGGT | 20 | SpyCas9 | 166 |
| TGFbR2096 | TTTTCCTTCATAATGCACTT | 20 | SpyCas9 | 167 |
| TGFBR24024 | CACAGTTGTGGAAACTTGAC | 20 | AsCpf1 | 168 |
| TGFBR24039 | CCCAACTCCGTCTTCCGCTC | 20 | AsCpf1 | 169 |
| TGFBR24040 | GGCTTTCCCTGCGTCTGGAC | 20 | AsCpf1 | 170 |
| TGFBR24036 | CTGAGGTCTATAAGGCCAAG | 20 | AsCpf1 | 171 |
| TGFBR24026 | TGATGTGAGATTTTCCACCT | 20 | AsCpf1 | 172 |
| TGFBR24038 | CCTATGAGGAGTATGCCTCT | 20 | AsCpf1 | 173 |
| TGFBR24033 | AAGTGACAGGCATCAGCCTC | 20 | AsCpf1 | 174 |
| TGFBR24028 | CCATGACCCCAAGCTCCCCT | 20 | AsCpf1 | 175 |
| TGFBR24031 | CTTCATAATGCACTTTGGAG | 20 | AsCpf1 | 176 |
| TGFBR24032 | TTCATGTGTTCCTGTAGCTC | 20 | AsCpf1 | 177 |
| TGFBR24029 | TTCTGGAAGATGCTGCTTCT | 20 | AsCpf1 | 178 |
| TGFBR24035 | CCCACCAGGGTGTCCAGCTC | 20 | AsCpf1 | 179 |
| TGFBR24037 | AGACAGTGGCAGTCAAGATC | 20 | AsCpf1 | 180 |
| TGFBR24041 | CCTGCGTCTGGACCCTACTC | 20 | AsCpf1 | 181 |
| TGFBR24025 | CACAACTGTGTAAATTTTGT | 20 | AsCpf1 | 182 |
| TGFBR24030 | GAGAAGCAGCATCTTCCAGA | 20 | AsCpf1 | 183 |
| TGFBR24027 | TGGTTGTCACAGGTGGAAAA | 20 | AsCpf1 | 184 |
| TGFBR24034 | CCAGGTTGAACTCAGCTTCT | 20 | AsCpf1 | 185 |
| TGFBR24043 | ATCACAAAATTTACACAGTTG | 21 | SauCas9 | 186 |
| TGFBR24065 | GGCATCAGCCTCCTGCCACCA | 21 | SauCas9 | 187 |
| TGFBR24110 | GTTAGCCAGGTCATCCACAGA | 21 | SauCas9 | 188 |
| TGFBR24099 | GCTGGGCAGCTCCCTCGCCCG | 21 | SauCas9 | 189 |
| TGFBR24064 | CAGGAGGCTGATGCCTGTCAC | 21 | SauCas9 | 190 |
| TGFBR24094 | GAGGAGCGGAAGACGGAGTTG | 21 | SauCas9 | 191 |
| TGFBR24108 | CGTCTGGACCCTACTCTGTCT | 21 | SauCas9 | 192 |
| TGFBR24058 | TTTTTCCTTCATAATGCACTT | 21 | SauCas9 | 193 |
| TGFBR24075 | CCATTGAGCTGGACACCCTGG | 21 | SauCas9 | 194 |
| TGFBR24057 | CTTCTCCAAAGTGCATTATGA | 21 | SauCas9 | 195 |
| TGFBR24103 | GCCCAAGATGCCCATCGTGCA | 21 | SauCas9 | 196 |
| TGFBR24060 | TCATGTGTTCCTGTAGCTCTG | 21 | SauCas9 | 197 |
| TGFBR24048 | GTGATGCTGCAGTTGCTCATG | 21 | SauCas9 | 198 |
| TGFBR24087 | TCTCATGCTTCAGATTGATGT | 21 | SauCas9 | 199 |
| TGFBR24081 | TCCCTATGAGGAGTATGCCTC | 21 | SauCas9 | 200 |
| TGFBR24044 | CATCACAAAATTTACACAGTT | 21 | SauCas9 | 201 |
| TGFBR24077 | ATTGAGCTGGACACCCTGGTG | 21 | SauCas9 | 202 |
| TGFBR24080 | CAGTCAAGATCTTTCCCTATG | 21 | SauCas9 | 203 |
| TGFBR24046 | AGGATTTCTGGTTGTCACAGG | 21 | SauCas9 | 204 |
| TGFBR24101 | TCCACAGTGATCACACTCCAT | 21 | SauCas9 | 205 |
| TGFBR24079 | AGCAGAACACTTCAGAGCAGT | 21 | SauCas9 | 206 |
| TGFBR24072 | CCGGCAAGACGCGGAAGCTCA | 21 | SauCas9 | 207 |
| TGFBR24074 | GATGTCAGAGCGGTCATCTTC | 21 | SauCas9 | 208 |
| TGFBR24062 | TCATTGCACTCATCAGAGCTA | 21 | SauCas9 | 209 |
| TGFBR24054 | CTTCCAGAATAAAGTCATGGT | 21 | SauCas9 | 210 |
| TGFBR24045 | AGATTTTCCACCTGTGACAAC | 21 | SauCas9 | 211 |
| TGFBR24049 | ACTGCAGCATCACCTCCATCT | 21 | SauCas9 | 212 |
| TGFBR24098 | AGCTGGGCAGCTCCCTCGCCC | 21 | SauCas9 | 213 |
| TGFBR24090 | TGACGGCTGAGGAGCGGAAGA | 21 | SauCas9 | 214 |
| TGFBR24076 | CATTGAGCTGGACACCCTGGT | 21 | SauCas9 | 215 |
| TGFBR24078 | AGCAAAGCGACCTTTCCCCAC | 21 | SauCas9 | 216 |
| TGFBR24067 | CGCGTTAACCGGCAGCAGAAG | 21 | SauCas9 | 217 |
| TGFBR24063 | GAAATATGACTAGCAACAAGT | 21 | SauCas9 | 218 |
| TGFBR24107 | AGACAGAGTAGGGTCCAGACG | 21 | SauCas9 | 219 |
| TGFBR24047 | CAGGATTTCTGGTTGTCACAG | 21 | SauCas9 | 220 |
| TGFBR24096 | CTCCTGTAGGTTGCCCTTGGC | 21 | SauCas9 | 221 |
| TGFBR24105 | ACAGAGTAGGGTCCAGACGCA | 21 | SauCas9 | 222 |
| TGFBR24056 | GCTTCTCCAAAGTGCATTATG | 21 | SauCas9 | 223 |
| TGFBR24068 | GCAGCAGAAGCTGAGTTCAAC | 21 | SauCas9 | 224 |
| TGFBR24093 | TGAGGAGCGGAAGACGGAGTT | 21 | SauCas9 | 225 |
| TGFBR24055 | CTTTGGAGAAGCAGCATCTTC | 21 | SauCas9 | 226 |
| TGFBR24053 | CTCCCCTACCATGACTTTATT | 21 | SauCas9 | 227 |
| TGFBR24106 | GACAGAGTAGGGTCCAGACGC | 21 | SauCas9 | 228 |
| TGFBR24092 | CTGAGGAGCGGAAGACGGAGT | 21 | SauCas9 | 229 |
| TGFBR24102 | GGGCATCTTGGGCCTCCCACA | 21 | SauCas9 | 230 |
| TGFBR24082 | CCAAGAGGCATACTCCTCATA | 21 | SauCas9 | 231 |
| TGFBR24051 | AGAATGACGAGAACATAACAC | 21 | SauCas9 | 232 |
| TGFBR24097 | CCTGACGCGGCATGTCATCAG | 21 | SauCas9 | 233 |
| TGFBR24073 | AGCGAGCACTGTGCCATCATC | 21 | SauCas9 | 234 |
| TGFBR24104 | GCAGGTTAGGTCGTTCTTCAC | 21 | SauCas9 | 235 |
| TGFBR24050 | ACCTCCATCTGTGAGAAGCCA | 21 | SauCas9 | 236 |
| TGFBR24052 | TAAAGTCATGGTAGGGGAGCT | 21 | SauCas9 | 237 |
| TGFBR24061 | TCAGAGCTACAGGAACACATG | 21 | SauCas9 | 238 |
| TGFBR24086 | TCTCAGACATCAATCTGAAGC | 21 | SauCas9 | 239 |
| TGFBR24066 | CATCAGCCTCCTGCCACCACT | 21 | SauCas9 | 240 |
| TGFBR24089 | CGCTCCTCAGCCGTCAGGAAC | 21 | SauCas9 | 241 |
| TGFBR24071 | AACCTGGGAAACCGGCAAGAC | 21 | SauCas9 | 242 |
| TGFBR24095 | TCCACGCCAAGGGCAACCTAC | 21 | SauCas9 | 243 |
| TGFBR24100 | GAGGTGAGCAATCCCCCGGGC | 21 | SauCas9 | 244 |
| TGFBR24069 | CAGCAGAAGCTGAGTTCAACC | 21 | SauCas9 | 245 |
| TGFBR24083 | TCCAAGAGGCATACTCCTCAT | 21 | SauCas9 | 246 |
| TGFBR24070 | AGCAGAAGCTGAGTTCAACCT | 21 | SauCas9 | 247 |
| TGFBR24088 | CCAGTTCCTGACGGCTGAGGA | 21 | SauCas9 | 248 |
| TGFBR24085 | AGGAGTATGCCTCTTGGAAGA | 21 | SauCas9 | 249 |
| TGFBR24084 | TTCCAAGAGGCATACTCCTCA | 21 | SauCas9 | 250 |
| TGFBR24042 | CAACTGTGTAAATTTTGTGAT | 21 | SauCas9 | 251 |
| TGFBR24059 | TGAAGGAAAAAAAAAAGCCTG | 21 | SauCas9 | 252 |
| TGFBR24091 | CGTCTTCCGCTCCTCAGCCGT | 21 | SauCas9 | 253 |
| TGFBR24109 | CCAGGTCATCCACAGACAGAG | 21 | SauCas9 | 254 |
| TGFBR2736 | GCCTAGAGTGAAGAGATTCAT | 21 | SpyCas9 | 255 |
| TGFBR2737 | GTTCTCCAAAGTGCATTATGA | 21 | SpyCas9 | 256 |
| TGFBR2738 | GCATCTTCCAGAATAAAGTCA | 21 | SpyCas9 | 257 |
| TGFBR2739 | TGATGTGAGATTTTCCACCTG | 21 | Cas12a | 1172 |

In some embodiments the gRNA for use in the disclosure is a gRNA targeting CISH (CISH gRNA). In some embodiments, the gRNA targeting CISH is one or more of the gRNAs described in Table 5.

TABLE 5
Exemplary CISH gRNAs

| | gRNA Targeting | | | SEQ |
|---|---|---|---|---|
| | Domain Sequence | | | ID |
| Name | (DNA) | Length | Enzyme | NO: |
| CISH0873 | CAACCGTCTGGTGGCCGACG | 20 | SpyCas9 | 258 |
| CISH0874 | CAGGATCGGGGCTGTCGCTT | 20 | SpyCas9 | 259 |
| CISH0875 | TCGGGCCTCGCTGGCCGTAA | 20 | SpyCas9 | 260 |
| CISH0876 | GAGGTAGTCGGCCATGCGCC | 20 | SpyCas9 | 261 |
| CISH0877 | CAGGTGTTGTCGGGCCTCGC | 20 | SpyCas9 | 262 |
| CISH0878 | GGAGGTAGTCGGCCATGCGC | 20 | SpyCas9 | 263 |
| CISH0879 | GGCATACTCAATGCGTACAT | 20 | SpyCas9 | 264 |
| CISH0880 | CCGCCTTGTCATCAACCGTC | 20 | SpyCas9 | 265 |
| CISH0881 | AGGATCGGGGCTGTCGCTTC | 20 | SpyCas9 | 266 |
| CISH0882 | CCTTGTCATCAACCGTCTGG | 20 | SpyCas9 | 267 |
| CISH0883 | TACTCAATGCGTACATTGGT | 20 | SpyCas9 | 268 |
| CISH0884 | GGGTTCCATTACGGCCAGCG | 20 | SpyCas9 | 269 |
| CISH0885 | GGCACTGCTTCTGCGTACAA | 20 | SpyCas9 | 270 |
| CISH0886 | GGTTGATGACAAGGCGGCAC | 20 | SpyCas9 | 271 |
| CISH0887 | TGCTGGGGCCTTCCTCGAGG | 20 | SpyCas9 | 272 |
| CISH0888 | TTGCTGGCTGTGGAGCGGAC | 20 | SpyCas9 | 273 |
| CISH0889 | TTCTCCTACCTTCGGGAATC | 20 | SpyCas9 | 274 |
| CISH0890 | GACTGGCTTGGGCAGTTCCA | 20 | SpyCas9 | 275 |
| CISH0891 | CATGCAGCCCTTGCCTGCTG | 20 | SpyCas9 | 276 |
| CISH0892 | AGCAAAGGACGAGGTCTAGA | 20 | SpyCas9 | 277 |
| CISH0893 | GCCTGCTGGGGCCTTCCTCG | 20 | SpyCas9 | 278 |
| CISH0894 | CAGACTCACCAGATTCCCGA | 20 | SpyCas9 | 279 |
| CISH0895 | ACCTCGTCCTTTGCTGGCTG | 20 | SpyCas9 | 280 |
| CISH0896 | CTCACCAGATTCCCGAAGGT | 20 | SpyCas9 | 281 |
| CISH7048 | TACGCAGAAGCAGTGCCCGC | 20 | AsCpf1 | 282 |
| CISH7049 | AGGTGTACAGCAGTGGCTGG | 20 | AsCpf1 | 283 |
| CISH7050 | GGTGTACAGCAGTGGCTGGT | 20 | AsCpf1 | 284 |
| CISH7051 | CGGATGTGGTCAGCCTTGTG | 20 | AsCpf1 | 285 |
| CISH7052 | CACTGACAGCGTGAACAGGT | 20 | AsCpf1 | 286 |
| CISH7053 | ACTGACAGCGTGAACAGGTA | 20 | AsCpf1 | 287 |
| CISH7054 | GCTCACTCTCTGTCTGGGCT | 20 | AsCpf1 | 288 |
| CISH7055 | CTGGCTGTGGAGCGGACTGG | 20 | AsCpf1 | 289 |
| CISH7056 | GCTCTGACTGTACGGGGCAA | 20 | AsCpf1 RR | 290 |
| CISH7057 | AGCTCTGACTGTACGGGGCA | 20 | AsCpf1 RR | 291 |
| CISH7058 | ACAGTACCCCTTCCAGCTCT | 20 | AsCpf1 RR | 292 |
| CISH7059 | CGTCGGCCACCAGACGGTTG | 20 | AsCpf1 RR | 293 |
| CISH7060 | CCAGCCACTGCTGTACACCT | 20 | AsCpf1 RR | 294 |
| CISH7061 | ACCCCGGCCCTGCCTATGCC | 20 | AsCpf1 RR | 295 |
| CISH7062 | GGTATCAGCAGTGCAGGAGG | 20 | AsCpf1 RR | 296 |
| CISH7063 | GATGTGGTCAGCCTTGTGCA | 20 | AsCpf1 RR | 297 |
| CISH7064 | GGATGTGGTCAGCCTTGTGC | 20 | AsCpf1 RR | 298 |
| CISH7065 | GGCCACGCATCCTGGCCTTT | 20 | AsCpf1 RR | 299 |
| CISH7066 | GAAAGGCCAGGATGCGTGGC | 20 | AsCpf1 RR | 300 |
| CISH7067 | ACTGCTTGTCCAGGCCACGC | 20 | AsCpf1 RR | 301 |
| CISH7068 | TCTGGACTCCAACTGCTTGT | 20 | AsCpf1 RR | 302 |
| CISH7069 | GTCTGGACTCCAACTGCTTG | 20 | AsCpf1 RR | 303 |
| CISH7070 | GCTTCCGTCTGGACTCCAAC | 20 | AsCpf1 RR | 304 |
| CISH7071 | GACGGAAGCTGGAGTCGGCA | 20 | AsCpf1 RR | 305 |
| CISH7072 | CGCTGTCAGTGAAAACCACT | 20 | AsCpf1 RR | 306 |
| CISH7073 | CTGACAGCGTGAACAGGTAG | 20 | AsCpf1 RR | 307 |
| CISH7074 | TTACGGCCAGCGAGGCCCGA | 20 | AsCpf1 RR | 308 |
| CISH7075 | ATTACGGCCAGCGAGGCCCG | 20 | AsCpf1 RR | 309 |
| CISH7076 | GGAATCTGGTGAGTCTGAGG | 20 | AsCpf1 RR | 310 |
| CISH7077 | CCCTCAGACTCACCAGATTC | 20 | AsCpf1 RR | 311 |
| CISH7078 | CGAAGGTAGGAGAAGGTCTT | 20 | AsCpf1 RR | 312 |
| CISH7079 | GAAGGTAGGAGAAGGTCTTG | 20 | AsCpf1 RR | 313 |
| CISH7080 | GCACCTTTGGCTCACTCTCT | 20 | AsCpf1 RR | 314 |
| CISH7081 | TCGAGGAGGTGGCAGAGGGT | 20 | AsCpf1 RR | 315 |
| CISH7082 | TGGAACTGCCCAAGCCAGTC | 20 | AsCpf1 RR | 316 |
| CISH7083 | AGGGACGGGGCCCACAGGGG | 20 | AsCpf1 RR | 317 |
| CISH7084 | GGGACGGGGCCCACAGGGGC | 20 | AsCpf1 RR | 318 |
| CISH7085 | CTCCACAGCCAGCAAAGGAC | 20 | AsCpf1 RR | 319 |
| CISH7086 | CAGCCAGCAAAGGACGAGGT | 20 | AsCpf1 RR | 320 |
| CISH7087 | CTGCCTTCTAGACCTCGTCC | 20 | AsCpf1 RR | 321 |
| CISH7088 | CCTAAGGAGGATGCGCCTAG | 20 | AsCpf | 322 |
| | | | RVR | |
| CISH7089 | TGGCCTCCTGCACTGCTGAT | 20 | AsCpf | 323 |
| | | | RVR | |
| CISH7090 | AGCAGTGCAGGAGGCCACAT | 20 | AsCpf | 324 |
| | | | RVR | |
| CISH7091 | CCGACTCCAGCTTCCGTCTG | 20 | AsCpf | 325 |
| | | | RVR | |
| CISH7092 | GGGGTTCCATTACGGCCAGC | 20 | AsCpf | 326 |
| | | | RVR | |
| CISH7093 | CACAGCAGATCCTCCTCTGG | 20 | AsCpf | 327 |
| | | | RVR | |
| CISH7094 | ATTGCCCCGTACAGTCAGAG | 20 | SauCas9 | 328 |
| CISH7095 | CCCGTACAGTCAGAGCTGGA | 20 | SauCas9 | 329 |
| CISH7096 | TGGTGGAGGAGCAGGCAGTG | 20 | SauCas9 | 330 |
| CISH7097 | TCCTTAGGCATAGGCAGGGC | 20 | SauCas9 | 331 |
| CISH7098 | CGGCCCTGCCTATGCCTAAG | 20 | SauCas9 | 332 |
| CISH7099 | TAGGCATAGGCAGGGCCGGG | 20 | SauCas9 | 333 |
| CISH7100 | AGGCAGGGCCGGGGTGGGAG | 20 | SauCas9 | 334 |
| CISH7101 | GCAGGATCGGGGCTGTCGCT | 20 | SauCas9 | 335 |
| CISH7102 | CTGCACAAGGCTGACCACAT | 20 | SauCas9 | 336 |
| CISH7103 | TGCACAAGGCTGACCACATC | 20 | SauCas9 | 337 |
| CISH7104 | CTGACCACATCCGGAAAGGC | 20 | SauCas9 | 338 |
| CISH7105 | GGCCACGCATCCTGGCCTTT | 20 | SauCas9 | 339 |
| CISH7106 | GCGTGGCCTGGACAAGCAGT | 20 | SauCas9 | 340 |
| CISH7107 | GACAAGCAGTTGGAGTCCAG | 20 | SauCas9 | 341 |
| CISH7108 | GTTGGAGTCCAGACGGAAGC | 20 | SauCas9 | 342 |
| CISH7109 | ATGCGTACATTGGTGGGGCC | 20 | SauCas9 | 343 |
| CISH7110 | TGGCCCCACCAATGTACGCA | 20 | SauCas9 | 344 |
| CISH7111 | GCTACCTGTTCACGCTGTCA | 20 | SauCas9 | 345 |
| CISH7112 | TGACAGCGTGAACAGGTAGC | 20 | SauCas9 | 346 |
| CISH7113 | GTCGGGCCTCGCTGGCCGTA | 20 | SauCas9 | 347 |
| CISH7114 | GCACTTGCCTAGGCTGGTAT | 20 | SauCas9 | 348 |
| CISH7115 | GGGAATCTGGTGAGTCTGAG | 20 | SauCas9 | 349 |
| CISH7116 | CTCACCAGATTCCCGAAGGT | 20 | SauCas9 | 350 |
| CISH7117 | CTCCTACCTTCGGGAATCTG | 20 | SauCas9 | 351 |
| CISH7118 | CAAGACCTTCTCCTACCTTC | 20 | SauCas9 | 352 |
| CISH7119 | CCAAGACCTTCTCCTACCTT | 20 | SauCas9 | 353 |
| CISH7120 | GCCAAGACCTTCTCCTACCT | 20 | SauCas9 | 354 |
| CISH7121 | TATGCACAGCAGATCCTCCT | 20 | SauCas9 | 355 |
| CISH7122 | CAAAGGTGCTGGACCCAGAG | 20 | SauCas9 | 356 |
| CISH7123 | GGCTCACTCTCTGTCTGGGC | 20 | SauCas9 | 357 |
| CISH7124 | AGGGTACCCCAGCCCAGACA | 20 | SauCas9 | 358 |
| CISH7125 | AGAGGGTACCCCAGCCCAGA | 20 | SauCas9 | 359 |
| CISH7126 | GTACCCTCTGCCACCTCCTC | 20 | SauCas9 | 360 |
| CISH7127 | CCTTCCTCGAGGAGGTGGCA | 20 | SauCas9 | 361 |
| CISH7128 | ATGACTGGCTTGGGCAGTTC | 20 | SauCas9 | 362 |
| CISH7129 | GGCCCCTGTGGGCCCCGTCC | 20 | SauCas9 | 363 |
| CISH7130 | AGGACGAGGTCTAGAAGGCA | 20 | SauCas9 | 364 |
| CISH7131 | ACTGACAGCGTGAACAGGTAG | 21 | Cas12a | 1173 |

In some embodiments, the gRNA for use in the disclosure is a gRNA targeting B2M (B2M gRNA). In some embodiments, the gRNA targeting B2M is one or more of the gRNAs described in Table 6.

TABLE 6
Exemplary B2M gRNAs

| | gRNA Targeting | | | SEQ |
|---|---|---|---|---|
| gRNA | Domain Target | | | ID |
| name | sequence (DNA) | Length | Enzyme | NO: |
| B2M1 | TATAAGTGGAGGCGTCGCGC | 20 | SpyCas9 | 365 |
| B2M2 | GGGCACGCGTTTAATATAAG | 20 | SpyCas9 | 366 |
| B2M3 | ACTCACGCTGGATAGCCTCC | 20 | SpyCas9 | 367 |
| B2M4 | GGCCGAGATGTCTCGCTCCG | 20 | SpyCas9 | 368 |
| B2M5 | CACGCGTTTAATATAAGTGG | 20 | SpyCas9 | 369 |
| B2M6 | AAGTGGAGGCGTCGCGCTGG | 20 | SpyCas9 | 370 |
| B2M7 | GAGTAGCGCGAGCACAGCTA | 20 | SpyCas9 | 371 |
| B2M8 | AGTGGAGGCGTCGCGCTGGC | 20 | SpyCas9 | 372 |
| B2M9 | GCCCGAATGCTGTCAGCTTC | 20 | SpyCas9 | 373 |
| B2M10 | CGCGAGCACAGCTAAGGCCA | 20 | SpyCas9 | 374 |
| B2M11 | CTCGCGCTACTCTCTCTTTC | 20 | SpyCas9 | 375 |
| B2M12 | GGCCACGGAGCGAGACATCT | 20 | SpyCas9 | 376 |
| B2M13 | CGTGAGTAAACCTGAATCTT | 20 | SpyCas9 | 377 |
| B2M14 | AGTCACATGGTTCACACGGC | 20 | SpyCas9 | 378 |
| B2M15 | AAGTCAACTTCAATGTCGGA | 20 | SpyCas9 | 379 |
| B2M16 | CAGTAAGTCAACTTCAATGT | 20 | SpyCas9 | 380 |
| B2M17 | ACCCAGACACATAGCAATTC | 20 | SpyCas9 | 381 |
| B2M18 | GCATACTCATCTTTTTCAGT | 20 | SpyCas9 | 382 |
| B2M19 | ACAGCCCAAGATAGTTAAGT | 20 | SpyCas9 | 383 |
| B2M20 | GGCATACTCATCTTTTTCAG | 20 | SpyCas9 | 384 |
| B2M21 | TTCCTGAAGCTGACAGCATT | 20 | SpyCas9 | 385 |
| B2M22 | TCACGTCATCCAGCAGAGAA | 20 | SpyCas9 | 386 |
| B2M23 | CAGCCCAAGATAGTTAAGTG | 20 | SpyCas9 | 387 |
| B2M-c1 | AATTCTCTCTCCATTCTT | 18 | AsCpf1 | 388 |
| B2M-c2 | AATTCTCTCTCCATTCTTC | 19 | AsCpf1 | 389 |
| B2M-c3 | AATTCTCTCTCCATTCTTCA | 20 | AsCpf1 | 390 |
| B2M-c4 | AATTCTCTCTCCATTCTTCA | 21 | AsCpf1 | 391 |
| | G | | | |
| B2M-c5 | AATTCTCTCTCCATTCTTCA | 22 | AsCpf1 | 392 |
| | GT | | | |
| B2M-c6 | AATTCTCTCTCCATTCTTCA | 23 | AsCpf1 | 393 |
| | GTA | | | |
| B2M-c7 | AATTCTCTCTCCATTCTTCA | 24 | AsCpf1 | 394 |
| | GTAA | | | |
| B2M-c8 | ACTTTCCATTCTCTGCTG | 18 | AsCpf1 | 395 |
| B2M-c9 | ACTTTCCATTCTCTGCTGG | 19 | AsCpf1 | 396 |
| B2M-c10 | ACTTTCCATTCTCTGCTGGA | 20 | AsCpf1 | 397 |
| B2M-c11 | ACTTTCCATTCTCTGCTGGA | 21 | AsCpf1 | 398 |
| | T | | | |
| B2M-c12 | ACTTTCCATTCTCTGCTGGA | 22 | AsCpf1 | 399 |
| | TG | | | |
| B2M-c13 | ACTTTCCATTCTCTGCTGGA | 23 | AsCpf1 | 400 |
| | TGA | | | |
| B2M-c14 | ACTTTCCATTCTCTGCTGGA | 24 | AsCpf1 | 401 |
| | TGAC | | | |
| B2M-c15 | AGCAAGGACTGGTCTTTC | 18 | AsCpf1 | 402 |
| B2M-c16 | AGCAAGGACTGGTCTTTCT | 19 | AsCpf1 | 403 |
| B2M-c17 | AGCAAGGACTGGTCTTTCTA | 20 | AsCpf1 | 404 |
| B2M-c18 | AGCAAGGACTGGTCTTTCTA | 21 | AsCpf1 | 405 |
| | T | | | |
| B2M-c19 | AGCAAGGACTGGTCTTTCTA | 22 | AsCpf1 | 406 |
| | TC | | | |
| B2M-c20 | AGCAAGGACTGGTCTTTCTA | 23 | AsCpf1 | 407 |
| | TCT | | | |
| B2M-c21 | AGCAAGGACTGGTCTTTCTA | 24 | AsCpf1 | 408 |
| | TCTC | | | |
| B2M-c22 | AGTGGGGGTGAATTCAGT | 18 | AsCpf1 | 409 |
| B2M-c23 | AGTGGGGGTGAATTCAGTG | 19 | AsCpf1 | 410 |
| B2M-c24 | AGTGGGGGTGAATTCAGTGT | 20 | AsCpf1 | 411 |
| B2M-c25 | AGTGGGGGTGAATTCAGTGT | 21 | AsCpf1 | 412 |
| | A | | | |
| B2M-c26 | AGTGGGGGTGAATTCAGTGT | 22 | AsCpf1 | 413 |
| | AG | | | |
| B2M-c27 | AGTGGGGGTGAATTCAGTGT | 23 | AsCpf1 | 414 |
| | AGT | | | |
| B2M-c28 | AGTGGGGGTGAATTCAGTGT | 24 | AsCpf1 | 415 |
| | AGTA | | | |
| B2M-c29 | ATCCATCCGACATTGAAG | 18 | AsCpf1 | 416 |
| B2M-c30 | ATCCATCCGACATTGAAGT | 19 | AsCpf1 | 417 |
| B2M-c31 | ATCCATCCGACATTGAAGTT | 20 | AsCpf1 | 418 |
| B2M-c32 | ATCCATCCGACATTGAAGTT | 21 | AsCpf1 | 419 |
| | G | | | |
| B2M-c33 | ATCCATCCGACATTGAAGTT | 22 | AsCpf1 | 420 |
| | GA | | | |
| B2M-c34 | ATCCATCCGACATTGAAGTT | 23 | AsCpf1 | 421 |
| | GAC | | | |
| B2M-c35 | ATCCATCCGACATTGAAGTT | 24 | AsCpf1 | 422 |
| | GACT | | | |
| B2M-c36 | CAATTCTCTCTCCATTCT | 18 | AsCpf1 | 423 |
| B2M-c37 | CAATTCTCTCTCCATTCTT | 19 | AsCpf1 | 424 |
| B2M-c38 | CAATTCTCTCTCCATTCTTC | 20 | AsCpf1 | 425 |
| B2M-c39 | CAATTCTCTCTCCATTCTTC | 21 | AsCpf1 | 426 |
| | A | | | |
| B2M-c40 | CAATTCTCTCTCCATTCTTC | 22 | AsCpf1 | 427 |
| | AG | | | |
| B2M-c41 | CAATTCTCTCTCCATTCTTC | 23 | AsCpf1 | 428 |
| | AGT | | | |
| B2M-c42 | CAATTCTCTCTCCATTCTTC | 24 | AsCpf1 | 429 |
| | AGTA | | | |
| B2M-c43 | CAGTGGGGGTGAATTCAG | 18 | AsCpf1 | 430 |
| B2M-c44 | CAGTGGGGGTGAATTCAGT | 19 | AsCpf1 | 431 |
| B2M-c45 | CAGTGGGGGTGAATTCAGTG | 20 | AsCpf1 | 432 |
| B2M-c46 | CAGTGGGGGTGAATTCAGTG | 21 | AsCpf1 | 433 |
| | T | | | |
| B2M-c47 | CAGTGGGGGTGAATTCAGTG | 22 | AsCpf1 | 434 |
| | TA | | | |
| B2M-c48 | CAGTGGGGGTGAATTCAGTG | 23 | AsCpf1 | 435 |
| | TAG | | | |
| B2M-c49 | CAGTGGGGGTGAATTCAGTG | 24 | AsCpf1 | 436 |
| | TAGT | | | |
| B2M-c50 | CATTCTCTGCTGGATGAC | 18 | AsCpf1 | 437 |
| B2M-c51 | CATTCTCTGCTGGATGACG | 19 | AsCpf1 | 438 |
| B2M-c52 | CATTCTCTGCTGGATGACGT | 20 | AsCpf1 | 439 |
| B2M-c53 | CATTCTCTGCTGGATGACGT | 21 | AsCpf1 | 440 |
| | G | | | |
| B2M-c54 | CATTCTCTGCTGGATGACGT | 22 | AsCpf1 | 441 |
| | GA | | | |
| B2M-c55 | CATTCTCTGCTGGATGACGT | 23 | AsCpf1 | 442 |
| | GAG | | | |
| B2M-c56 | CATTCTCTGCTGGATGACGT | 24 | AsCpf1 | 443 |
| | GAGT | | | |
| B2M-c57 | CCCGATATTCCTCAGGTA | 18 | AsCpf1 | 444 |
| B2M-c58 | CCCGATATTCCTCAGGTAC | 19 | AsCpf1 | 445 |
| B2M-c59 | CCCGATATTCCTCAGGTACT | 20 | AsCpf1 | 446 |
| B2M-c60 | CCCGATATTCCTCAGGTACT | 21 | AsCpf1 | 447 |
| | C | | | |
| B2M-c61 | CCCGATATTCCTCAGGTACT | 22 | AsCpf1 | 448 |
| | CC | | | |
| B2M-c62 | CCCGATATTCCTCAGGTACT | 23 | AsCpf1 | 449 |
| | CCA | | | |
| B2M-c63 | CCCGATATTCCTCAGGTACT | 24 | AsCpf1 | 450 |
| | CCAA | | | |
| B2M-c64 | CCGATATTCCTCAGGTAC | 18 | AsCpf1 | 451 |
| B2M-c65 | CCGATATTCCTCAGGTACT | 19 | AsCpf1 | 452 |
| B2M-c66 | CCGATATTCCTCAGGTACTC | 20 | AsCpf1 | 453 |
| B2M-c67 | CCGATATTCCTCAGGTACTC | 21 | AsCpf1 | 454 |
| | C | | | |
| B2M-c68 | CCGATATTCCTCAGGTACTC | 22 | AsCpf1 | 455 |
| | CA | | | |
| B2M-c69 | CCGATATTCCTCAGGTACTC | 23 | AsCpf1 | 456 |
| | CAA | | | |
| B2M-c70 | CCGATATTCCTCAGGTACTC | 24 | AsCpf1 | 457 |
| | CAAA | | | |
| B2M-c71 | CTCACGTCATCCAGCAGA | 18 | AsCpf1 | 458 |
| B2M-c72 | CTCACGTCATCCAGCAGAG | 19 | AsCpf1 | 459 |
| B2M-c73 | CTCACGTCATCCAGCAGAGA | 20 | AsCpf1 | 460 |
| B2M-c74 | CTCACGTCATCCAGCAGAGA | 21 | AsCpf1 | 461 |
| | A | | | |
| B2M-c75 | CTCACGTCATCCAGCAGAGA | 22 | AsCpf1 | 462 |
| | AT | | | |
| B2M-c76 | CTCACGTCATCCAGCAGAGA | 23 | AsCpf1 | 463 |
| | ATG | | | |
| B2M-c77 | CTCACGTCATCCAGCAGAGA | 24 | AsCpf1 | 464 |
| | ATGG | | | |
| B2M-c78 | CTGAATTGCTATGTGTCT | 18 | AsCpf1 | 465 |
| B2M-c79 | CTGAATTGCTATGTGTCTG | 19 | AsCpf1 | 466 |
| B2M-c80 | CTGAATTGCTATGTGTCTGG | 20 | AsCpf1 | 467 |
| B2M-c81 | CTGAATTGCTATGTGTCTGG | 21 | AsCpf1 | 468 |
| | G | | | |
| B2M-c82 | CTGAATTGCTATGTGTCTGG | 22 | AsCpf1 | 469 |
| | GT | | | |
| B2M-c83 | CTGAATTGCTATGTGTCTGG | 23 | AsCpf1 | 470 |
| | GTT | | | |
| B2M-c84 | CTGAATTGCTATGTGTCTGG | 24 | AsCpf1 | 471 |
| | GTTT | | | |
| B2M-c85 | GAGTACCTGAGGAATATC | 18 | AsCpf1 | 472 |
| B2M-c86 | GAGTACCTGAGGAATATCG | 19 | AsCpf1 | 473 |
| B2M-c87 | GAGTACCTGAGGAATATCGG | 20 | AsCpf1 | 474 |
| B2M-c88 | GAGTACCTGAGGAATATCGG | 21 | AsCpf1 | 475 |
| | G | | | |
| B2M-c89 | GAGTACCTGAGGAATATCGG | 22 | AsCpf1 | 476 |
| | GA | | | |
| B2M-c90 | GAGTACCTGAGGAATATCGG | 23 | AsCpf1 | 477 |
| | GAA | | | |
| B2M-c91 | GAGTACCTGAGGAATATCGG | 24 | AsCpf1 | 478 |
| | GAAA | | | |
| B2M-c92 | TATCTCTTGTACTACACT | 18 | AsCpf1 | 479 |
| B2M-c93 | TATCTCTTGTACTACACTG | 19 | AsCpf1 | 480 |
| B2M-c94 | TATCTCTTGTACTACACTGA | 20 | AsCpf1 | 481 |
| B2M-c95 | TATCTCTTGTACTACACTGA | 21 | AsCpf1 | 482 |
| | A | | | |
| B2M-c96 | TATCTCTTGTACTACACTGA | 22 | AsCpf1 | 483 |
| | AT | | | |
| B2M-c97 | TATCTCTTGTACTACACTGA | 23 | AsCpf1 | 484 |
| | ATT | | | |
| B2M-c98 | TATCTCTTGTACTACACTGA | 24 | AsCpf1 | 485 |
| | ATTC | | | |
| B2M-c99 | TCAATTCTCTCTCCATTC | 18 | AsCpf1 | 486 |
| B2M-c100 | TCAATTCTCTCTCCATTCT | 19 | AsCpf1 | 487 |
| B2M-c101 | TCAATTCTCTCTCCATTCTT | 20 | AsCpf1 | 488 |
| B2M-c102 | TCAATTCTCTCTCCATTCTT | 21 | AsCpf1 | 489 |
| | C | | | |
| B2M-c103 | TCAATTCTCTCTCCATTCTT | 22 | AsCpf1 | 490 |
| | CA | | | |
| B2M-c104 | TCAATTCTCTCTCCATTCTT | 23 | AsCpf1 | 491 |
| | CAG | | | |
| B2M-c105 | TCAATTCTCTCTCCATTCTT | 24 | AsCpf1 | 492 |
| | CAGT | | | |
| B2M-c106 | TCACAGCCCAAGATAGTT | 18 | AsCpf1 | 493 |
| B2M-c107 | TCACAGCCCAAGATAGTTA | 19 | AsCpf1 | 494 |
| B2M-c108 | TCACAGCCCAAGATAGTTAA | 20 | AsCpf1 | 495 |
| B2M-c109 | TCACAGCCCAAGATAGTTAA | 21 | AsCpf1 | 496 |
| | G | | | |
| B2M-c110 | TCACAGCCCAAGATAGTTAA | 22 | AsCpf1 | 497 |
| | GT | | | |
| B2M-c111 | TCACAGCCCAAGATAGTTAA | 23 | AsCpf1 | 498 |
| | GTG | | | |
| B2M-c112 | TCACAGCCCAAGATAGTTAA | 24 | AsCpf1 | 499 |
| | GTGG | | | |
| B2M-c113 | TCAGTGGGGGTGAATTCA | 18 | AsCpf1 | 500 |
| B2M-c114 | TCAGTGGGGGTGAATTCAG | 19 | AsCpf1 | 501 |
| B2M-c115 | TCAGTGGGGGTGAATTCAGT | 20 | AsCpf1 | 502 |
| B2M-c116 | TCAGTGGGGGTGAATTCAGT | 21 | AsCpf1 | 503 |
| | G | | | |
| B2M-c117 | TCAGTGGGGGTGAATTCAGT | 22 | AsCpf1 | 504 |
| | GT | | | |
| B2M-c118 | TCAGTGGGGGTGAATTCAGT | 23 | AsCpf1 | 505 |
| | GTA | | | |
| B2M-c119 | TCAGTGGGGGTGAATTCAGT | 24 | AsCpf1 | 506 |
| | GTAG | | | |
| B2M-c120 | TGGCCTGGAGGCTATCCA | 18 | AsCpf1 | 507 |
| B2M-c121 | TGGCCTGGAGGCTATCCAG | 19 | AsCpf1 | 508 |
| B2M-c122 | TGGCCTGGAGGCTATCCAGC | 20 | AsCpf1 | 509 |
| B2M-c123 | TGGCCTGGAGGCTATCCAGC | 21 | AsCpf1 | 510 |
| | G | | | |
| B2M-c124 | TGGCCTGGAGGCTATCCAGC | 22 | AsCpf1 | 511 |
| | GT | | | |
| B2M-c125 | TGGCCTGGAGGCTATCCAGC | 23 | AsCpf1 | 512 |
| | GTG | | | |
| B2M-c126 | TGGCCTGGAGGCTATCCAGC | 24 | AsCpf1 | 513 |
| | GTGA | | | |
| B2M-c127 | ATAGATCGAGACATGTAA | 18 | AsCpf1 | 514 |
| B2M-c128 | ATAGATCGAGACATGTAAG | 19 | AsCpf1 | 515 |
| B2M-c129 | ATAGATCGAGACATGTAAGC | 20 | AsCpf1 | 516 |
| B2M-c130 | ATAGATCGAGACATGTAAGC | 21 | AsCpf1 | 517 |
| | A | | | |
| B2M-c131 | ATAGATCGAGACATGTAAGC | 22 | AsCpf1 | 518 |
| | AG | | | |
| B2M-c132 | ATAGATCGAGACATGTAAGC | 23 | AsCpf1 | 519 |
| | AGC | | | |
| B2M-c133 | ATAGATCGAGACATGTAAGC | 24 | AsCpf1 | 520 |
| | AGCA | | | |
| B2M-c134 | CATAGATCGAGACATGTA | 18 | AsCpf1 | 521 |
| B2M-c135 | CATAGATCGAGACATGTAA | 19 | AsCpf1 | 522 |
| B2M-c136 | CATAGATCGAGACATGTAAG | 20 | AsCpf1 | 523 |
| B2M-c137 | CATAGATCGAGACATGTAAG | 21 | AsCpf1 | 524 |
| | C | | | |
| B2M-c138 | CATAGATCGAGACATGTAAG | 22 | AsCpf1 | 525 |
| | CA | | | |
| B2M-c139 | CATAGATCGAGACATGTAAG | 23 | AsCpf1 | 526 |
| | CAG | | | |
| B2M-c140 | CATAGATCGAGACATGTAAG | 24 | AsCpf1 | 527 |
| | CAGC | | | |
| B2M-c141 | CTCCACTGTCTTTTTCAT | 18 | AsCpf1 | 528 |
| B2M-c142 | CTCCACTGTCTTTTTCATA | 19 | AsCpf1 | 529 |
| B2M-c143 | CTCCACTGTCTTTTTCATAG | 20 | AsCpf1 | 530 |
| B2M-c144 | CTCCACTGTCTTTTTCATAG | 21 | AsCpf1 | 531 |
| | A | | | |
| B2M-c145 | CTCCACTGTCTTTTTCATAG | 22 | AsCpf1 | 532 |
| | AT | | | |
| B2M-c146 | CTCCACTGTCTTTTTCATAG | 23 | AsCpf1 | 533 |
| | ATC | | | |
| B2M-c147 | CTCCACTGTCTTTTTCATAG | 24 | AsCpf1 | 534 |
| | ATCG | | | |
| B2M-c148 | TCATAGATCGAGACATGT | 18 | AsCpf1 | 535 |
| B2M-c149 | TCATAGATCGAGACATGTA | 19 | AsCpf1 | 536 |
| B2M-c150 | TCATAGATCGAGACATGTAA | 20 | AsCpf1 | 537 |
| B2M-c151 | TCATAGATCGAGACATGTAA | 21 | AsCpf1 | 538 |
| | G | | | |
| B2M-c152 | TCATAGATCGAGACATGTAA | 22 | AsCpf1 | 539 |
| | GC | | | |
| B2M-c153 | TCATAGATCGAGACATGTAA | 23 | AsCpf1 | 540 |
| | GCA | | | |
| B2M-c154 | TCATAGATCGAGACATGTAA | 24 | AsCpf1 | 541 |
| | GCAG | | | |
| B2M-c155 | TCCACTGTCTTTTTCATA | 18 | AsCpf1 | 542 |
| B2M-c156 | TCCACTGTCTTTTTCATAG | 19 | AsCpf1 | 543 |
| B2M-c157 | TCCACTGTCTTTTTCATAGA | 20 | AsCpf1 | 544 |
| B2M-c158 | TCCACTGTCTTTTTCATAGA | 21 | AsCpf1 | 545 |
| | T | | | |
| B2M-c159 | TCCACTGTCTTTTTCATAGA | 22 | AsCpf1 | 546 |
| | TC | | | |
| B2M-c160 | TCCACTGTCTTTTTCATAGA | 23 | AsCpf1 | 547 |
| | TCG | | | |
| B2M-c161 | TCCACTGTCTTTTTCATAGA | 24 | AsCpf1 | 548 |
| | TCGA | | | |
| B2M-c162 | TCTCCACTGTCTTTTTCA | 18 | AsCpf1 | 549 |
| B2M-c163 | TCTCCACTGTCTTTTTCAT | 19 | AsCpf1 | 550 |
| B2M-c164 | TCTCCACTGTCTTTTTCATA | 20 | AsCpf1 | 551 |
| B2M-c165 | TCTCCACTGTCTTTTTCATA | 21 | AsCpf1 | 552 |
| | G | | | |
| B2M-c166 | TCTCCACTGTCTTTTTCATA | 22 | AsCpf1 | 553 |
| | GA | | | |
| B2M-c167 | TCTCCACTGTCTTTTTCATA | 23 | AsCpf1 | 554 |
| | GAT | | | |
| B2M-c168 | TCTCCACTGTCTTTTTCATA | 24 | AsCpf1 | 555 |
| | GATC | | | |
| B2M-c169 | TTCTCCACTGTCTTTTTC | 18 | AsCpf1 | 556 |
| B2M-c170 | TTCTCCACTGTCTTTTTCA | 19 | AsCpf1 | 557 |
| B2M-c171 | TTCTCCACTGTCTTTTTCAT | 20 | AsCpf1 | 558 |
| B2M-c172 | TTCTCCACTGTCTTTTTCAT | 21 | AsCpf1 | 559 |
| | A | | | |
| B2M-c173 | TTCTCCACTGTCTTTTTCAT | 22 | AsCpf1 | 560 |
| | AG | | | |
| B2M-c174 | TTCTCCACTGTCTTTTTCAT | 23 | AsCpf1 | 561 |
| | AGA | | | |
| B2M-c175 | TTCTCCACTGTCTTTTTCAT | 24 | AsCpf1 | 562 |
| | AGAT | | | |
| B2M-c176 | TTTCTCCACTGTCTTTTT | 18 | AsCpf1 | 563 |
| B2M-c177 | TTTCTCCACTGTCTTTTTC | 19 | AsCpf1 | 564 |
| B2M-c178 | TTTCTCCACTGTCTTTTTCA | 20 | AsCpf1 | 565 |
| B2M-c179 | TTTCTCCACTGTCTTTTTCA | 21 | AsCpf1 | 566 |
| | T | | | |
| B2M-c180 | TTTCTCCACTGTCTTTTTCA | 22 | AsCpf1 | 567 |
| | TA | | | |
| B2M-c181 | TTTCTCCACTGTCTTTTTCA | 23 | AsCpf1 | 568 |
| | TAG | | | |
| B2M-c182 | TTTCTCCACTGTCTTTTTCA | 24 | AsCpf1 | 569 |
| | TAGA | | | |
| B2M-c183 | TTTTCTCCACTGTCTTTT | 18 | AsCpf1 | 570 |
| B2M-c184 | TTTTCTCCACTGTCTTTTT | 19 | AsCpf1 | 571 |
| B2M-c185 | TTTTCTCCACTGTCTTTTTC | 20 | AsCpf1 | 572 |
| B2M-c186 | TTTTCTCCACTGTCTTTTTC | 21 | AsCpf1 | 573 |
| | A | | | |
| B2M-c187 | TTTTCTCCACTGTCTTTTTC | 22 | AsCpf1 | 574 |
| | AT | | | |
| B2M-c188 | TTTTCTCCACTGTCTTTTTC | 23 | AsCpf1 | 575 |
| | ATA | | | |
| B2M-c189 | TTTTCTCCACTGTCTTTTTC | 24 | AsCpf1 | 576 |
| | ATAG | | | |

In some embodiments, the gRNA for use in the disclosure is a gRNA targeting PD1. gRNAs targeting B2M and PD1 for use in the disclosure are further described in WO2015161276 and WO2017152015 by Welstead et al.; both incorporated in their entirety herein by reference.

In some embodiments, the gRNA for use in the disclosure is a gRNA targeting NKG2A (NKG2A gRNA). In some embodiments, the gRNA targeting NKG2A is one or more of the gRNAs described in Table 7.

TABLE 7
Exemplary NKG2A gRNAs

| | gRNA Targeting | | | SEQ |
|---|---|---|---|---|
| | Domain Sequence | | | ID |
| Name | (DNA) | Length | Enzyme | NO: |
| NKG2A55 | GAGGTAAAGCGTTTGCATTTG | 21 | AsCpf1 | 577 |
| NKG2A56 | CCTCTAAAGCTTATGCTTACA | 21 | AsCpf1 | 578 |
| NKG2A57 | AGTCGATTTACTTGTAGCACT | 21 | AsCpf1 | 579 |
| NKG2A58 | CTTGTAGCACTGCACAGTTAA | 21 | AsCpf1 | 580 |
| NKG2A59 | TCCATTACAGGATAAAAGACT | 21 | AsCpf1 | 581 |
| NKG2A60 | CTCCATTACAGGATAAAAGAC | 21 | AsCpf1 | 582 |
| NKG2A61 | TCTCCATTACAGGATAAAAGA | 21 | AsCpf1 | 583 |
| NKG2A62 | ATCCTGTAATGGAGAAAAATC | 21 | AsCpf1 | 584 |
| NKG2A63 | TCCTGTAATGGAGAAAAATCC | 21 | AsCpf1 | 585 |
| NKG2A136 | AAACATGAGTAAGTTGTTTTG | 21 | AsCpf1 | 586 |
| NKG2A137 | GCTTTCAAACATGAGTAAGTT | 21 | AsCpf1 | 587 |
| NKG2A138 | AAAGCCAAACCATTCATTGTC | 21 | AsCpf1 | 588 |
| NKG2A139 | GTAACAGCAGTCATCATCCAT | 21 | AsCpf1 | 589 |
| NKG2A140 | ACCATCCTCATGGATTGGTGT | 21 | AsCpf1 | 590 |
| NKG2A141 | TGTCCATCATTTCACCATCCT | 21 | AsCpf1 | 591 |
| NKG2A142 | GAAATTTCTGTCCATCATTTC | 21 | AsCpf1 | 592 |
| NKG2A143 | AGAAATTTCTGTCCATCATTT | 21 | AsCpf1 | 593 |
| NKG2A144 | TTTTAGAAATTTCTGTCCATC | 21 | AsCpf1 | 594 |
| NKG2A145 | CTTTTAGAAATTTCTGTCCAT | 21 | AsCpf1 | 595 |
| NKG2A146 | TTTTCTTTTAGAAATTTCTGT | 21 | AsCpf1 | 596 |
| NKG2A147 | TAAAAGAAAAGAAAGAATTTT | 21 | AsCpf1 | 597 |
| NKG2A270 | AAACATTTACATCTTACCATT | 21 | AsCpf1 | 598 |
| NKG2A271 | CATCTTACCATTTCTTCTTCA | 21 | AsCpf1 | 599 |
| NKG2A272 | TATAGATAATGAAGAAGAAAT | 21 | AsCpf1 | 600 |
| NKG2A273 | TTCTTCATTATCTATAGAAAG | 21 | AsCpf1 | 601 |
| NKG2A274 | CTGGCCTGTACTTCGAAGAAC | 21 | AsCpf1 | 602 |
| NKG2A275 | CTTACCAATGTAGTAACAACT | 21 | AsCpf1 | 603 |
| NKG2A276 | GCACGTCATTGTGGCCATTGT | 21 | AsCpf1 | 604 |
| NKG2A277 | TTTAGCACGTCATTGTGGCCA | 21 | AsCpf1 | 605 |
| NKG2A414 | CCATCAGCTCCAGAGAAGCTC | 21 | AsCpf1 | 606 |
| NKG2A415 | TCTCCCTGCAGATTTACCATC | 21 | AsCpf1 | 607 |
| NKG2A437 | AAATGCTTTACCTTTGCAGTG | 21 | AsCpf1 | 608 |
| NKG2A438 | AATGCTTTACCTTTGCAGTGA | 21 | AsCpf1 | 609 |
| NKG2A439 | CCTTTGCAGTGATAGGTTTTG | 21 | AsCpf1 | 610 |
| NKG2A440 | CAGTGATAGGTTTTGTCATTC | 21 | AsCpf1 | 611 |
| NKG2A441 | AAGGGAATGACAAAACCTATC | 21 | AsCpf1 | 612 |
| NKG2A442 | CAAGGGAATGACAAAACCTAT | 21 | AsCpf1 | 613 |
| NKG2A443 | GTCATTCCCTTGAAAATCCTG | 21 | AsCpf1 | 614 |
| NKG2A444 | TCATTCCCTTGAAAATCCTGA | 21 | AsCpf1 | 615 |
| NKG2A445 | TGAAGGTTTAATTCCGCATAG | 21 | AsCpf1 | 616 |
| NKG2A446 | GAAGGTTTAATTCCGCATAGG | 21 | AsCpf1 | 617 |
| NKG2A447 | AAGGTTTAATTCCGCATAGGT | 21 | AsCpf1 | 618 |
| NKG2A448 | ATTCCGCATAGGTTATTTCCT | 21 | AsCpf1 | 619 |
| NKG2A449 | GCAACTGAACAGGAAATAACC | 21 | AsCpf1 | 620 |
| NKG2A450 | AGCAACTGAACAGGAAATAAC | 21 | AsCpf1 | 621 |
| NKG2A451 | CTGTTCAGTTGCTAAAATGGA | 21 | AsCpf1 | 622 |
| NKG2A452 | TATTGCCTTTAGGTTTTCGTT | 21 | AsCpf1 | 623 |
| NKG2A453 | ATTGCCTTTAGGTTTTCGTTG | 21 | AsCpf1 | 624 |
| NKG2A454 | TTGCCTTTAGGTTTTCGTTGC | 21 | AsCpf1 | 625 |
| NKG2A455 | GGTTTTCGTTGCTGCCTCTTT | 21 | AsCpf1 | 626 |
| NKG2A456 | CGTTGCTGCCTCTTTGGGTTT | 21 | AsCpf1 | 627 |
| NKG2A457 | GTTGCTGCCTCTTTGGGTTTG | 21 | AsCpf1 | 628 |
| NKG2A458 | GGTTTGGGGGCAGATTCAGGT | 21 | AsCpf1 | 629 |
| NKG2A459 | GGGGCAGATTCAGGTCTGAGT | 21 | AsCpf1 | 630 |
| NKG2A460 | GCAACTGAACAGGAAATAACC | 21 | Cas12a | 1176 |

In some embodiments, the gRNA for use in the disclosure is a gRNA targeting TIGIT (TIGIT gRNA). In some embodiments, the gRNA targeting TIGIT is one or more of the gRNAs described in Table 8.

TABLE 8
Exemplary TIGIT gRNAs

| | gRNA Targeting | | | SEQ |
|---|---|---|---|---|
| | Domain Sequence | | | ID |
| Name | (DNA) | Length | Enzyme | NO: |
| TIGIT4170 | TCTGCAGAAATGTTCCCCGT | 20 | AsCpf1 | 631 |
| TIGIT4171 | TGCAGAGAAAGGTGGCTCTA | 20 | AsCpf1 | 632 |
| TIGIT4172 | TAATGCTGACTTGGGGTGGC | 20 | AsCpf1 | 633 |
| TIGIT4173 | TAGGACCTCCAGGAAGATTC | 20 | AsCpf1 | 634 |
| TIGIT4174 | TAGTCAACGCGACCACCACG | 20 | AsCpf1 | 635 |
| TIGIT4175 | TCCTGAGGTCACCTTCCACA | 20 | AsCpf1 | 636 |
| TIGIT4176 | TATTGTGCCTGTCATCATTC | 20 | AsCpf1 | 637 |
| TIGIT4177 | TGACAGGCACAATAGAAACAA | 21 | SauCas9 | 638 |
| TIGIT4178 | GACAGGCACAATAGAAACAAC | 21 | SauCas9 | 639 |
| TIGIT4179 | AAACAACGGGGAACATTTCTG | 21 | SauCas9 | 640 |
| TIGIT4180 | ACAACGGGGAACATTTCTGCA | 21 | SauCas9 | 641 |
| TIGIT4181 | TGATAGAGCCACCTTTCTCTG | 21 | SauCas9 | 642 |
| TIGIT4182 | GGGTCACTTGTGCCGTGGTGG | 21 | SauCas9 | 643 |
| TIGIT4183 | GGCACAAGTGACCCAGGTCAA | 21 | SauCas9 | 644 |
| TIGIT4184 | GTCCTGCTGCTCCCAGTTGAC | 21 | SauCas9 | 645 |
| TIGIT4185 | TGGCCATTTGTAATGCTGACT | 21 | SauCas9 | 646 |
| TIGIT4186 | TGGCACATCTCCCCATCCTTC | 21 | SauCas9 | 647 |
| TIGIT4187 | CATCTCCCCATCCTTCAAGGA | 21 | SauCas9 | 648 |
| TIGIT4188 | CCACTCGATCCTTGAAGGATG | 21 | SauCas9 | 649 |
| TIGIT4189 | GGCCACTCGATCCTTGAAGGA | 21 | SauCas9 | 650 |
| TIGIT4190 | CCTGGGGCCACTCGATCCTTG | 21 | SauCas9 | 651 |
| TIGIT4191 | GACTGGAGGGTGAGGCCCAGG | 21 | SauCas9 | 652 |
| TIGIT4192 | ATCGTTCACGGTCAGCGACTG | 21 | SauCas9 | 653 |
| TIGIT4193 | GTCGCTGACCGTGAACGATAC | 21 | SauCas9 | 654 |
| TIGIT4194 | CGCTGACCGTGAACGATACAG | 21 | SauCas9 | 655 |
| TIGIT4195 | GCATCTATCACACCTACCCTG | 21 | SauCas9 | 656 |
| TIGIT4196 | CCTACCCTGATGGGACGTACA | 21 | SauCas9 | 657 |
| TIGIT4197 | TACCCTGATGGGACGTACACT | 21 | SauCas9 | 658 |
| TIGIT4198 | CCCTGATGGGACGTACACTGG | 21 | SauCas9 | 659 |
| TIGIT4199 | TTCTCCCAGTGTACGTCCCAT | 21 | SauCas9 | 660 |
| TIGIT4200 | GGAGAATCTTCCTGGAGGTCC | 21 | SauCas9 | 661 |
| TIGIT4201 | CATGGCTCCAAGCAATGGAAT | 21 | SauCas9 | 662 |
| TIGIT4202 | CGCGGCCATGGCTCCAAGCAA | 21 | SauCas9 | 663 |
| TIGIT4203 | TCGCGGCCATGGCTCCAAGCA | 21 | SauCas9 | 664 |
| TIGIT4204 | CATCGTGGTGGTCGCGTTGAC | 21 | SauCas9 | 665 |
| TIGIT4205 | AAAGCCCTCAGAATCCATTCT | 21 | SauCas9 | 666 |
| TIGIT4206 | CATTCTGTGGAAGGTGACCTC | 21 | SauCas9 | 667 |
| TIGIT4207 | TTCTGTGGAAGGTGACCTCAG | 21 | SauCas9 | 668 |
| TIGIT4208 | CCTGAGGTCACCTTCCACAGA | 21 | SauCas9 | 669 |
| TIGIT4209 | TTCTCCTGAGGTCACCTTCCA | 21 | SauCas9 | 670 |
| TIGIT4210 | AGGAGAAAATCAGCTGGACAG | 21 | SauCas9 | 671 |
| TIGIT4211 | GGAGAAAATCAGCTGGACAGG | 21 | SauCas9 | 672 |
| TIGIT4212 | GCCCCAGTGCTCCCTCACCCC | 21 | SauCas9 | 673 |
| TIGIT4213 | TGGACACAGCTTCCTGGGGGT | 21 | SauCas9 | 674 |
| TIGIT4214 | TCTGCCTGGACACAGCTTCCT | 21 | SauCas9 | 675 |
| TIGIT4215 | AGCTGCACCTGCTGGGCTCTG | 21 | SauCas9 | 676 |
| TIGIT4216 | GCTGGGCTCTGTGGAGAGCAG | 21 | SauCas9 | 677 |
| TIGIT4217 | TGGGCTCTGTGGAGAGCAGCG | 21 | SauCas9 | 678 |
| TIGIT4218 | CTGCATGACTACTTCAATGTC | 21 | SauCas9 | 679 |
| TIGIT4219 | AATGTCCTGAGTTACAGAAGC | 21 | SauCas9 | 680 |
| TIGIT4220 | TGGGTAACTGCAGCTTCTTCA | 21 | SauCas9 | 681 |
| TIGIT4221 | GACAGGCACAATAGAAACAA | 20 | SpyCas9 | 682 |
| TIGIT4222 | ACAGGCACAATAGAAACAAC | 20 | SpyCas9 | 683 |
| TIGIT4223 | CAGGCACAATAGAAACAACG | 20 | SpyCas9 | 684 |
| TIGIT4224 | GGGAACATTTCTGCAGAGAA | 20 | SpyCas9 | 685 |
| TIGIT4225 | AACATTTCTGCAGAGAAAGG | 20 | SpyCas9 | 686 |
| TIGIT4226 | ATGTCACCTCTCCTCCACCA | 20 | SpyCas9 | 687 |
| TIGIT4227 | CTTGTGCCGTGGTGGAGGAG | 20 | SpyCas9 | 688 |
| TIGIT4228 | GGTCACTTGTGCCGTGGTGG | 20 | SpyCas9 | 689 |
| TIGIT4229 | CACCACGGCACAAGTGACCC | 20 | SpyCas9 | 690 |
| TIGIT4230 | CTGGGTCACTTGTGCCGTGG | 20 | SpyCas9 | 691 |
| TIGIT4231 | GACCTGGGTCACTTGTGCCG | 20 | SpyCas9 | 692 |
| TIGIT4232 | CACAAGTGACCCAGGTCAAC | 20 | SpyCas9 | 693 |
| TIGIT4233 | ACAAGTGACCCAGGTCAACT | 20 | SpyCas9 | 694 |
| TIGIT4234 | CCAGGTCAACTGGGAGCAGC | 20 | SpyCas9 | 695 |
| TIGIT4235 | CTGCTGCTCCCAGTTGACCT | 20 | SpyCas9 | 696 |
| TIGIT4236 | CCTGCTGCTCCCAGTTGACC | 20 | SpyCas9 | 697 |
| TIGIT4237 | GGAGCAGCAGGACCAGCTTC | 20 | SpyCas9 | 698 |
| TIGIT4238 | CATTACAAATGGCCAGAAGC | 20 | SpyCas9 | 699 |
| TIGIT4239 | GGCCATTTGTAATGCTGACT | 20 | SpyCas9 | 700 |
| TIGIT4240 | GCCATTTGTAATGCTGACTT | 20 | SpyCas9 | 701 |
| TIGIT4241 | CCATTTGTAATGCTGACTTG | 20 | SpyCas9 | 702 |
| TIGIT4242 | TTTGTAATGCTGACTTGGGG | 20 | SpyCas9 | 703 |
| TIGIT4243 | CCCCAAGTCAGCATTACAAA | 20 | SpyCas9 | 704 |
| TIGIT4244 | GCACATCTCCCCATCCTTCA | 20 | SpyCas9 | 705 |
| TIGIT4245 | CCCATCCTTCAAGGATCGAG | 20 | SpyCas9 | 706 |
| TIGIT4246 | CACTCGATCCTTGAAGGATG | 20 | SpyCas9 | 707 |
| TIGIT4247 | CCACTCGATCCTTGAAGGAT | 20 | SpyCas9 | 708 |
| TIGIT4248 | GCCACTCGATCCTTGAAGGA | 20 | SpyCas9 | 709 |
| TIGIT4249 | TTCAAGGATCGAGTGGCCCC | 20 | SpyCas9 | 710 |
| TIGIT4250 | TGGGGCCACTCGATCCTTGA | 20 | SpyCas9 | 711 |
| TIGIT4251 | GATCGAGTGGCCCCAGGTCC | 20 | SpyCas9 | 712 |
| TIGIT4252 | AGTGGCCCCAGGTCCCGGCC | 20 | SpyCas9 | 713 |
| TIGIT4253 | GTGGCCCCAGGTCCCGGCCT | 20 | SpyCas9 | 714 |
| TIGIT4254 | GAGGCCCAGGCCGGGACCTG | 20 | SpyCas9 | 715 |
| TIGIT4255 | TGAGGCCCAGGCCGGGACCT | 20 | SpyCas9 | 716 |
| TIGIT4256 | GTGAGGCCCAGGCCGGGACC | 20 | SpyCas9 | 717 |
| TIGIT4257 | TGGAGGGTGAGGCCCAGGCC | 20 | SpyCas9 | 718 |
| TIGIT4258 | CTGGAGGGTGAGGCCCAGGC | 20 | SpyCas9 | 719 |
| TIGIT4259 | GCGACTGGAGGGTGAGGCCC | 20 | SpyCas9 | 720 |
| TIGIT4260 | CGGTCAGCGACTGGAGGGTG | 20 | SpyCas9 | 721 |
| TIGIT4261 | GTTCACGGTCAGCGACTGGA | 20 | SpyCas9 | 722 |
| TIGIT4262 | CGTTCACGGTCAGCGACTGG | 20 | SpyCas9 | 723 |
| TIGIT4263 | TATCGTTCACGGTCAGCGAC | 20 | SpyCas9 | 724 |
| TIGIT4264 | TCGCTGACCGTGAACGATAC | 20 | SpyCas9 | 725 |
| TIGIT4265 | CGCTGACCGTGAACGATACA | 20 | SpyCas9 | 726 |
| TIGIT4266 | GCTGACCGTGAACGATACAG | 20 | SpyCas9 | 727 |
| TIGIT4267 | GTACTCCCCTGTATCGTTCA | 20 | SpyCas9 | 728 |
| TIGIT4268 | ATCTATCACACCTACCCTGA | 20 | SpyCas9 | 729 |
| TIGIT4269 | TCTATCACACCTACCCTGAT | 20 | SpyCas9 | 730 |
| TIGIT4270 | TACCCTGATGGGACGTACAC | 20 | SpyCas9 | 731 |
| TIGIT4271 | ACCCTGATGGGACGTACACT | 20 | SpyCas9 | 732 |
| TIGIT4272 | AGTGTACGTCCCATCAGGGT | 20 | SpyCas9 | 733 |
| TIGIT4273 | TCCCAGTGTACGTCCCATCA | 20 | SpyCas9 | 734 |
| TIGIT4274 | CTCCCAGTGTACGTCCCATC | 20 | SpyCas9 | 735 |
| TIGIT4275 | GTACACTGGGAGAATCTTCC | 20 | SpyCas9 | 736 |
| TIGIT4276 | CACTGGGAGAATCTTCCTGG | 20 | SpyCas9 | 737 |
| TIGIT4277 | CTGAGCTTTCTAGGACCTCC | 20 | SpyCas9 | 738 |
| TIGIT4278 | AGGTTCCAGATTCCATTGCT | 20 | SpyCas9 | 739 |
| TIGIT4279 | AAGCAATGGAATCTGGAACC | 20 | SpyCas9 | 740 |
| TIGIT4280 | GATTCCATTGCTTGGAGCCA | 20 | SpyCas9 | 741 |
| TIGIT4281 | TGGCTCCAAGCAATGGAATC | 20 | SpyCas9 | 742 |
| TIGIT4282 | GCGGCCATGGCTCCAAGCAA | 20 | SpyCas9 | 743 |
| TIGIT4283 | TGGAGCCATGGCCGCGACGC | 20 | SpyCas9 | 744 |
| TIGIT4284 | AGCCATGGCCGCGACGCTGG | 20 | SpyCas9 | 745 |
| TIGIT4285 | GACCACCAGCGTCGCGGCCA | 20 | SpyCas9 | 746 |
| TIGIT4286 | GCAGATGACCACCAGCGTCG | 20 | SpyCas9 | 747 |
| TIGIT4287 | CATCTGCACAGCAGTCATCG | 20 | SpyCas9 | 748 |
| TIGIT4288 | CTGCACAGCAGTCATCGTGG | 20 | SpyCas9 | 749 |
| TIGIT4289 | AGCCCTCAGAATCCATTCTG | 20 | SpyCas9 | 750 |
| TIGIT4290 | CTCAGAATCCATTCTGTGGA | 20 | SpyCas9 | 751 |
| TIGIT4291 | TTCCACAGAATGGATTCTGA | 20 | SpyCas9 | 752 |
| TIGIT4292 | CTTCCACAGAATGGATTCTG | 20 | SpyCas9 | 753 |
| TIGIT4293 | ATTCTGTGGAAGGTGACCTC | 20 | SpyCas9 | 754 |
| TIGIT4294 | TGAGGTCACCTTCCACAGAA | 20 | SpyCas9 | 755 |
| TIGIT4295 | GACCTCAGGAGAAAATCAGC | 20 | SpyCas9 | 756 |
| TIGIT4296 | CAGGAGAAAATCAGCTGGAC | 20 | SpyCas9 | 757 |
| TIGIT4297 | GTCCAGCTGATTTTCTCCTG | 20 | SpyCas9 | 758 |
| TIGIT4298 | GAGAAAATCAGCTGGACAGG | 20 | SpyCas9 | 759 |
| TIGIT4299 | AATCAGCTGGACAGGAGGAA | 20 | SpyCas9 | 760 |
| TIGIT4300 | CCCAGTGCTCCCTCACCCCC | 20 | SpyCas9 | 761 |
| TIGIT4301 | CTGGGGGTGAGGGAGCACTG | 20 | SpyCas9 | 762 |
| TIGIT4302 | CCTGGGGGTGAGGGAGCACT | 20 | SpyCas9 | 763 |
| TIGIT4303 | TCCTGGGGGTGAGGGAGCAC | 20 | SpyCas9 | 764 |
| TIGIT4304 | ACACAGCTTCCTGGGGGTGA | 20 | SpyCas9 | 765 |
| TIGIT4305 | GACACAGCTTCCTGGGGGTG | 20 | SpyCas9 | 766 |
| TIGIT4306 | ACCCCCAGGAAGCTGTGTCC | 20 | SpyCas9 | 767 |
| TIGIT4307 | GCCTGGACACAGCTTCCTGG | 20 | SpyCas9 | 768 |
| TIGIT4308 | TGCCTGGACACAGCTTCCTG | 20 | SpyCas9 | 769 |
| TIGIT4309 | CTGCCTGGACACAGCTTCCT | 20 | SpyCas9 | 770 |
| TIGIT4310 | TCTGCCTGGACACAGCTTCC | 20 | SpyCas9 | 771 |
| TIGIT4311 | CAGGCAGAAGCTGCACCTGC | 20 | SpyCas9 | 772 |
| TIGIT4312 | AGGCAGAAGCTGCACCTGCT | 20 | SpyCas9 | 773 |
| TIGIT4313 | CAGCAGGTGCAGCTTCTGCC | 20 | SpyCas9 | 774 |
| TIGIT4314 | GCTGCACCTGCTGGGCTCTG | 20 | SpyCas9 | 775 |
| TIGIT4315 | TGCTCTCCACAGAGCCCAGC | 20 | SpyCas9 | 776 |
| TIGIT4316 | CTGGGCTCTGTGGAGAGCAG | 20 | SpyCas9 | 777 |
| TIGIT4317 | TGGGCTCTGTGGAGAGCAGC | 20 | SpyCas9 | 778 |
| TIGIT4318 | GGGCTCTGTGGAGAGCAGCG | 20 | SpyCas9 | 779 |
| TIGIT4319 | CTGTGGAGAGCAGCGGGGAG | 20 | SpyCas9 | 780 |
| TIGIT4320 | ATTGAAGTAGTCATGCAGCT | 20 | SpyCas9 | 781 |
| TIGIT4321 | TGTCCTGAGTTACAGAAGCC | 20 | SpyCas9 | 782 |
| TIGIT4322 | GTCCTGAGTTACAGAAGCCT | 20 | SpyCas9 | 783 |
| TIGIT4323 | TACCCAGGCTTCTGTAACTC | 20 | SpyCas9 | 784 |
| TIGIT4324 | TGAAGAAGCTGCAGTTACCC | 20 | SpyCas9 | 785 |
| TIGIT4325 | TGCAGCTTCTTCACAGAGAC | 20 | SpyCas9 | 786 |
| TIGIT5053 | GTTGTTTCTATTGTGCCTGT | 20 | AsCpf1 RR | 787 |
| TIGIT5054 | CGTTGTTTCTATTGTGCCTG | 20 | AsCpf1 RR | 788 |
| TIGIT5055 | CCGTTGTTTCTATTGTGCCT | 20 | AsCpf1 RR | 789 |
| TIGIT5056 | CCACGGCACAAGTGACCCAG | 20 | AsCpf1 RR | 790 |
| TIGIT5057 | AGTTGACCTGGGTCACTTGT | 20 | AsCpf1 RR | 791 |
| TIGIT5058 | AAGTCAGCATTACAAATGGC | 20 | AsCpf1 RR | 792 |
| TIGIT5059 | CATCCTTCAAGGATCGAGTG | 20 | AsCpf1 RR | 793 |
| TIGIT5060 | ATCCTTCAAGGATCGAGTGG | 20 | AsCpf1 RR | 794 |
| TIGIT5061 | AGGATCGAGTGGCCCCAGGT | 20 | AsCpf1 RR | 795 |
| TIGIT5062 | AGGTCCCGGCCTGGGCCTCA | 20 | AsCpf1 RR | 796 |
| TIGIT5063 | GGCCTGGGCCTCACCCTCCA | 20 | AsCpf1 RR | 797 |
| TIGIT5064 | CGGTCAGCGACTGGAGGGTG | 20 | AsCpf1 RR | 798 |
| TIGIT5065 | GTCGCTGACCGTGAACGATA | 20 | AsCpf1 RR | 799 |
| TIGIT5066 | TGTATCGTTCACGGTCAGCG | 20 | AsCpf1 RR | 800 |
| TIGIT5067 | CTGTATCGTTCACGGTCAGC | 20 | AsCpf1 RR | 801 |
| TIGIT5068 | ATCAGGGTAGGTGTGATAGA | 20 | AsCpf1 RR | 802 |
| TIGIT5069 | AGTGTACGTCCCATCAGGGT | 20 | AsCpf1 RR | 803 |
| TIGIT5070 | GGAAGATTCTCCCAGTGTAC | 20 | AsCpf1 RR | 804 |
| TIGIT5071 | TGGAGGTCCTAGAAAGCTCA | 20 | AsCpf1 RR | 805 |
| TIGIT5072 | AGCAATGGAATCTGGAACCT | 20 | AsCpf1 RR | 806 |
| TIGIT5073 | AGATTCCATTGCTTGGAGCC | 20 | AsCpf1 RR | 807 |
| TIGIT5074 | GATTCCATTGCTTGGAGCCA | 20 | AsCpf1 RR | 808 |
| TIGIT5075 | ATTGCTTGGAGCCATGGCCG | 20 | AsCpf1 RR | 809 |
| TIGIT5076 | TTGCTTGGAGCCATGGCCGC | 20 | AsCpf1 RR | 810 |
| TIGIT5077 | CAGAATGGATTCTGAGGGCT | 20 | AsCpf1 RR | 811 |
| TIGIT5078 | ACAGAATGGATTCTGAGGGC | 20 | AsCpf1 RR | 812 |
| TIGIT5079 | TTCTGTGGAAGGTGACCTCA | 20 | AsCpf1 RR | 813 |
| TIGIT5080 | GCTGATTTTCTCCTGAGGTC | 20 | AsCpf1 RR | 814 |
| TIGIT5081 | TCCTGTCCAGCTGATTTTCT | 20 | AsCpf1 RR | 815 |
| TIGIT5082 | TTCCTCCTGTCCAGCTGATT | 20 | AsCpf1 RR | 816 |
| TIGIT5083 | TGGGGGTGAGGGAGCACTGG | 20 | AsCpf1 RR | 817 |
| TIGIT5084 | AGTGCTCCCTCACCCCCAGG | 20 | AsCpf1 RR | 818 |
| TIGIT5085 | TCACCCCCAGGAAGCTGTGT | 20 | AsCpf1 RR | 819 |
| TIGIT5086 | CAGGAAGCTGTGTCCAGGCA | 20 | AsCpf1 RR | 820 |
| TIGIT5087 | AGGAAGCTGTGTCCAGGCAG | 20 | AsCpf1 RR | 821 |
| TIGIT5088 | GGCAGAAGCTGCACCTGCTG | 20 | AsCpf1 RR | 822 |
| TIGIT5089 | CAGAGCCCAGCAGGTGCAGC | 20 | AsCpf1 RR | 823 |
| TIGIT5090 | GCTGCTCTCCACAGAGCCCA | 20 | AsCpf1 RR | 824 |
| TIGIT5091 | CGCTGCTCTCCACAGAGCCC | 20 | AsCpf1 RR | 825 |
| TIGIT5092 | ATGTCCTGAGTTACAGAAGC | 20 | AsCpf1 RR | 826 |
| TIGIT5093 | TGCAGAGAAAGGTGGCTCTAT | 21 | Cas12a | 1175 |

In some embodiments the gRNA for use in the disclosure is a gRNA targeting ADORA2a (ADORA2a gRNA). In some embodiments, the gRNA targeting ADORA2a is one or more of the gRNAs described in Table 9.

TABLE 9
Exemplary ADORA2a gRNAs

| | gRNA Targeting | | | SEQ |
|---|---|---|---|---|
| | Domain Sequence | | | ID |
| Name | (DNA) | Length | Enzyme | NO: |
| ADORA2A337 | GAGCACACCCACTGCGATGT | 20 | SpyCas9 | 827 |
| ADORA2A338 | GATGGCCAGGAGACTGAAGA | 20 | SpyCas9 | 828 |
| ADORA2A339 | CTGCTCACCGGAGCGGGATG | 20 | SpyCas9 | 829 |
| ADORA2A340 | GTCTGTGGCCATGCCCATCA | 20 | SpyCas9 | 830 |
| ADORA2A341 | TCACCGGAGCGGGATGCGGA | 20 | SpyCas9 | 831 |
| ADORA2A342 | GTGGCAGGCAGCGCAGAACC | 20 | SpyCas9 | 832 |
| ADORA2A343 | AGCACACCAGCACATTGCCC | 20 | SpyCas9 | 833 |
| ADORA2A344 | CAGGTTGCTGTTGAGCCACA | 20 | SpyCas9 | 834 |
| ADORA2A345 | CTTCATTGCCTGCTTCGTCC | 20 | SpyCas9 | 835 |
| ADORA2A346 | GTACACCGAGGAGCCCATGA | 20 | SpyCas9 | 836 |
| ADORA2A347 | GATGGCAATGTAGCGGTCAA | 20 | SpyCas9 | 837 |
| ADORA2A348 | CTCCTCGGTGTACATCACGG | 20 | SpyCas9 | 838 |
| ADORA2A349 | CGAGGAGCCCATGATGGGCA | 20 | SpyCas9 | 839 |
| ADORA2A350 | GGGCTCCTCGGTGTACATCA | 20 | SpyCas9 | 840 |
| ADORA2A351 | CTTTGTGGTGTCACTGGCGG | 20 | SpyCas9 | 841 |
| ADORA2A352 | CCGCTCCGGTGAGCAGGGCC | 20 | SpyCas9 | 842 |
| ADORA2A353 | GGGTTCTGCGCTGCCTGCCA | 20 | SpyCas9 | 843 |
| ADORA2A354 | GGACGAAGCAGGCAATGAAG | 20 | SpyCas9 | 844 |
| ADORA2A355 | GTGCTGATGGTGATGGCAAA | 20 | SpyCas9 | 845 |
| ADORA2A356 | AGCGCAGAACCCGGTGCTGA | 20 | SpyCas9 | 846 |
| ADORA2A357 | GAGCTCCATCTTCAGTCTCC | 20 | SpyCas9 | 847 |
| ADORA2A358 | TGCTGATGGTGATGGCAAAG | 20 | SpyCas9 | 848 |
| ADORA2A359 | GGCGGCGGCCGACATCGCAG | 20 | SpyCas9 | 849 |
| ADORA2A360 | AATGAAGAGGCAGCCGTGGC | 20 | SpyCas9 | 850 |
| ADORA2A361 | GGGCAATGTGCTGGTGTGCT | 20 | SpyCas9 | 851 |
| ADORA2A362 | CATGCCCATCATGGGCTCCT | 20 | SpyCas9 | 852 |
| ADORA2A363 | AATGTAGCGGTCAATGGCGA | 20 | SpyCas9 | 853 |
| ADORA2A364 | AGTAGTTGGTGACGTTCTGC | 20 | SpyCas9 | 854 |
| ADORA2A365 | AGCGGTCAATGGCGATGGCC | 20 | SpyCas9 | 855 |
| ADORA2A366 | CGCATCCCGCTCCGGTGAGC | 20 | SpyCas9 | 856 |
| ADORA2A367 | GCATCCCGCTCCGGTGAGCA | 20 | SpyCas9 | 857 |
| ADORA2A368 | TGGGCAATGTGCTGGTGTGC | 20 | SpyCas9 | 858 |
| ADORA2A369 | CAACTACTTTGTGGTGTCAC | 20 | SpyCas9 | 859 |
| ADORA2A370 | CGCTCCGGTGAGCAGGGCCG | 20 | SpyCas9 | 860 |
| ADORA2A371 | GATGGTGATGGCAAAGGGGA | 20 | SpyCas9 | 861 |
| ADORA2A372 | GGTGTACATCACGGTGGAGC | 20 | SpyCas9 | 862 |
| ADORA2A373 | GAACGTCACCAACTACTTTG | 20 | SpyCas9 | 863 |
| ADORA2A374 | CAGTGACACCACAAAGTAGT | 20 | SpyCas9 | 864 |
| ADORA2A375 | GGCCATCCTGGGCAATGTGC | 20 | SpyCas9 | 865 |
| ADORA2A376 | CCCGGCCCTGCTCACCGGAG | 20 | SpyCas9 | 866 |
| ADORA2A377 | CACCAGCACATTGCCCAGGA | 20 | SpyCas9 | 867 |
| ADORA2A378 | TTTGCCATCACCATCAGCAC | 20 | SpyCas9 | 868 |
| ADORA2A379 | CTCCACCGTGATGTACACCG | 20 | SpyCas9 | 869 |
| ADORA2A380 | GGAGCTGGCCATTGCTGTGC | 20 | SpyCas9 | 870 |
| ADORA2A381 | CAGGATGGCCAGCACAGCAA | 20 | SpyCas9 | 871 |
| ADORA2A382 | GAACCCGGTGCTGATGGTGA | 20 | SpyCas9 | 872 |
| ADORA2A383 | TGGAGCTCTGCGTGAGGACC | 20 | SpyCas9 | 873 |
| ADORA2A384 | CCCGCTCCGGTGAGCAGGGC | 20 | SpyCas9 | 874 |
| ADORA2A385 | AGGCAATGAAGAGGCAGCCG | 20 | SpyCas9 | 875 |
| ADORA2A386 | CCGGCCCTGCTCACCGGAGC | 20 | SpyCas9 | 876 |
| ADORA2A387 | GCGGCGGCCGACATCGCAGT | 20 | SpyCas9 | 877 |
| ADORA2A388 | GGTGCTGATGGTGATGGCAA | 20 | SpyCas9 | 878 |
| ADORA2A389 | CTACTTTGTGGTGTCACTGG | 20 | SpyCas9 | 879 |
| ADORA2A390 | TACACCGAGGAGCCCATGAT | 20 | SpyCas9 | 880 |
| ADORA2A391 | TCTGTGGCCATGCCCATCAT | 20 | SpyCas9 | 881 |
| ADORA2A392 | ATTGCTGTGCTGGCCATCCT | 20 | SpyCas9 | 882 |
| ADORA2A393 | CGTGAGGACCAGGACGAAGC | 20 | SpyCas9 | 883 |
| ADORA2A394 | TTGCCATCACCATCAGCACC | 20 | SpyCas9 | 884 |
| ADORA2A395 | GGATGCGGATGGCAATGTAG | 20 | SpyCas9 | 885 |
| ADORA2A396 | TTGCCATCCGCATCCCGCTC | 20 | SpyCas9 | 886 |
| ADORA2A397 | TGAAGATGGAGCTCTGCGTG | 20 | SpyCas9 | 887 |
| ADORA2A398 | CATTGCTGTGCTGGCCATCC | 20 | SpyCas9 | 888 |
| ADORA2A399 | TGCTGGTGTGCTGGGCCGTG | 20 | SpyCas9 | 889 |
| ADORA2A820 | GGCTCCTCGGTGTACATCACG | 21 | SauCas9 | 890 |
| ADORA2A821 | GAGCTCTGCGTGAGGACCAGG | 21 | SauCas9 | 891 |
| ADORA2A822 | GATGGAGCTCTGCGTGAGGAC | 21 | SauCas9 | 892 |
| ADORA2A823 | CCAGCACACCAGCACATTGCC | 21 | SauCas9 | 893 |
| ADORA2A824 | AGGACCAGGACGAAGCAGGCA | 21 | SauCas9 | 894 |
| ADORA2A825 | TGCCATCCGCATCCCGCTCCG | 21 | SauCas9 | 895 |
| ADORA2A826 | GTGTGGCTCAACAGCAACCTG | 21 | SauCas9 | 896 |
| ADORA2A827 | AGCTCCACCGTGATGTACACC | 21 | SauCas9 | 897 |
| ADORA2A828 | GTAGCGGTCAATGGCGATGGC | 21 | SauCas9 | 898 |
| ADORA2A829 | CGGTGCTGATGGTGATGGCAA | 21 | SauCas9 | 899 |
| ADORA2A830 | CCCTGCTCACCGGAGCGGGAT | 21 | SauCas9 | 900 |
| ADORA2A831 | GTGACGTTCTGCAGGTTGCTG | 21 | SauCas9 | 901 |
| ADORA2A832 | GCTCCACCGTGATGTACACCG | 21 | SauCas9 | 902 |
| ADORA2A833 | ACTGAAGATGGAGCTCTGCGT | 21 | SauCas9 | 903 |
| ADORA2A834 | CCAGCTCCACCGTGATGTACA | 21 | SauCas9 | 904 |
| ADORA2A835 | CCTTTGCCATCACCATCAGCA | 21 | SauCas9 | 905 |
| ADORA2A836 | CCGGTGCTGATGGTGATGGCA | 21 | SauCas9 | 906 |
| ADORA2A837 | CCTGGGCAATGTGCTGGTGTG | 21 | SauCas9 | 907 |
| ADORA2A838 | AGGCAGCCGTGGCAGGCAGCG | 21 | SauCas9 | 908 |
| ADORA2A839 | GCGATGGCCAGGAGACTGAAG | 21 | SauCas9 | 909 |
| ADORA2A840 | CGATGGCCAGGAGACTGAAGA | 21 | SauCas9 | 910 |
| ADORA2A841 | TCCCGCTCCGGTGAGCAGGGC | 21 | SauCas9 | 911 |
| ADORA2A842 | TGCTTCGTCCTGGTCCTCACG | 21 | SauCas9 | 912 |
| ADORA2A843 | ACCAGGACGAAGCAGGCAATG | 21 | SauCas9 | 913 |
| ADORA2A844 | ATGTACACCGAGGAGCCCATG | 21 | SauCas9 | 914 |
| ADORA2A845 | TCGTCTGTGGCCATGCCCATC | 21 | SauCas9 | 915 |
| ADORA2A846 | TCAATGGCGATGGCCAGGAGA | 21 | SauCas9 | 916 |
| ADORA2A847 | GGTGCTGATGGTGATGGCAAA | 21 | SauCas9 | 917 |
| ADORA2A848 | TAGCGGTCAATGGCGATGGCC | 21 | SauCas9 | 918 |
| ADORA2A849 | TCCGCATCCCGCTCCGGTGAG | 21 | SauCas9 | 919 |
| ADORA2A850 | CTGGCGGCGGCCGACATCGCA | 21 | SauCas9 | 920 |
| ADORA2A851 | GCCATTGCTGTGCTGGCCATC | 21 | SauCas9 | 921 |
| ADORA2A852 | ATCCCGCTCCGGTGAGCAGGG | 21 | SauCas9 | 922 |
| ADORA2A853 | AGACTGAAGATGGAGCTCTGC | 21 | SauCas9 | 923 |
| ADORA2A854 | CCCCGGCCCTGCTCACCGGAG | 21 | SauCas9 | 924 |
| ADORA2A855 | ATGGTGATGGCAAAGGGGATG | 21 | SauCas9 | 925 |
| ADORA2A856 | GCTCCTCGGTGTACATCACGG | 21 | SauCas9 | 926 |
| ADORA2A248 | TGTCGATGGCAATAGCCAAG | 20 | SpyCas9 | 927 |
| ADORA2A249 | AGAAGTTGGTGACGTTCTGC | 20 | SpyCas9 | 928 |
| ADORA2A250 | TTCGCCATCACCATCAGCAC | 20 | SpyCas9 | 929 |
| ADORA2A251 | GAAGAAGAGGCAGCCATGGC | 20 | SpyCas9 | 930 |
| ADORA2A252 | CACAAGCACGTTACCCAGGA | 20 | SpyCas9 | 931 |
| ADORA2A253 | CAACTTCTTCGTGGTATCTC | 20 | SpyCas9 | 932 |
| ADORA2A254 | CAGGATGGCCAGCACAGCAA | 20 | SpyCas9 | 933 |
| ADORA2A255 | AATTCCACTCCGGTGAGCCA | 20 | SpyCas9 | 934 |
| ADORA2A256 | AGCGCAGAAGCCAGTGCTGA | 20 | SpyCas9 | 935 |
| ADORA2A257 | GTGCTGATGGTGATGGCGAA | 20 | SpyCas9 | 936 |
| ADORA2A258 | GGAGCTGGCCATTGCTGTGC | 20 | SpyCas9 | 937 |
| ADORA2A259 | AATAGCCAAGAGGCTGAAGA | 20 | SpyCas9 | 938 |
| ADORA2A260 | CTCCTCGGTGTACATCATGG | 20 | SpyCas9 | 939 |
| ADORA2A261 | GGACAAAGCAGGCGAAGAAG | 20 | SpyCas9 | 940 |
| ADORA2A262 | TCTGGCGGCGGCTGACATCG | 20 | SpyCas9 | 941 |
| ADORA2A263 | TGGGTAACGTGCTTGTGTGC | 20 | SpyCas9 | 942 |
| ADORA2A264 | GATGTACACCGAGGAGCCCA | 20 | SpyCas9 | 943 |
| ADORA2A265 | TAACCCCTGGCTCACCGGAG | 20 | SpyCas9 | 944 |
| ADORA2A266 | TCACCGGAGTGGAATTCGGA | 20 | SpyCas9 | 945 |
| ADORA2A267 | GCGGCGGCTGACATCGCGGT | 20 | SpyCas9 | 946 |
| ADORA2A268 | GATGGTGATGGCGAATGGGA | 20 | SpyCas9 | 947 |
| ADORA2A269 | GGCTTCTGCGCTGCCTGCCA | 20 | SpyCas9 | 948 |
| ADORA2A270 | ATTCCACTCCGGTGAGCCAG | 20 | SpyCas9 | 949 |
| ADORA2A271 | GGTGTACATCATGGTGGAGC | 20 | SpyCas9 | 950 |
| ADORA2A272 | ATTGCTGTGCTGGCCATCCT | 20 | SpyCas9 | 951 |
| ADORA2A273 | CTCCACCATGATGTACACCG | 20 | SpyCas9 | 952 |
| ADORA2A274 | GGCGGCGGCTGACATCGCGG | 20 | SpyCas9 | 953 |
| ADORA2A275 | TACACCGAGGAGCCCATGGC | 20 | SpyCas9 | 954 |
| ADORA2A276 | GGGTAACGTGCTTGTGTGCT | 20 | SpyCas9 | 955 |
| ADORA2A277 | CAGGTTGCTGTTGATCCACA | 20 | SpyCas9 | 956 |
| ADORA2A278 | TGAAGATGGAACTCTGCGTG | 20 | SpyCas9 | 957 |
| ADORA2A279 | GATGGCGATGTATCTGTCGA | 20 | SpyCas9 | 958 |
| ADORA2A280 | CTTCTTCGCCTGCTTTGTCC | 20 | SpyCas9 | 959 |
| ADORA2A281 | AGGCGAAGAAGAGGCAGCCA | 20 | SpyCas9 | 960 |
| ADORA2A282 | TGCTTGTGTGCTGGGCCGTG | 20 | SpyCas9 | 961 |
| ADORA2A283 | GAAGCCAGTGCTGATGGTGA | 20 | SpyCas9 | 962 |
| ADORA2A284 | CGTGAGGACCAGGACAAAGC | 20 | SpyCas9 | 963 |
| ADORA2A285 | TGGAACTCTGCGTGAGGACC | 20 | SpyCas9 | 964 |
| ADORA2A286 | CATTGCTGTGCTGGCCATCC | 20 | SpyCas9 | 965 |
| ADORA2A287 | TTCTCCCGCCATGGGCTCCT | 20 | SpyCas9 | 966 |
| ADORA2A288 | TGGCTCACCGGAGTGGAATT | 20 | SpyCas9 | 967 |
| ADORA2A289 | TGCTGATGGTGATGGCGAAT | 20 | SpyCas9 | 968 |
| ADORA2A290 | CTTCGTGGTATCTCTGGCGG | 20 | SpyCas9 | 969 |
| ADORA2A291 | AGCACACAAGCACGTTACCC | 20 | SpyCas9 | 970 |
| ADORA2A292 | GGGCTCCTCGGTGTACATCA | 20 | SpyCas9 | 971 |
| ADORA2A293 | GTACACCGAGGAGCCCATGG | 20 | SpyCas9 | 972 |
| ADORA2A294 | GAACGTCACCAACTTCTTCG | 20 | SpyCas9 | 973 |
| ADORA2A295 | TCGCCATCCGAATTCCACTC | 20 | SpyCas9 | 974 |
| ADORA2A296 | GAGTTCCATCTTCAGCCTCT | 20 | SpyCas9 | 975 |
| ADORA2A297 | GAATTCCACTCCGGTGAGCC | 20 | SpyCas9 | 976 |
| ADORA2A298 | CAGAGATACCACGAAGAAGT | 20 | SpyCas9 | 977 |
| ADORA2A299 | CTTCTTCGTGGTATCTCTGG | 20 | SpyCas9 | 978 |
| ADORA2A695 | CAGTGCTGATGGTGATGGCGA | 21 | SauCas9 | 979 |
| ADORA2A696 | CGAATTCCACTCCGGTGAGCC | 21 | SauCas9 | 980 |
| ADORA2A697 | CCGAATTCCACTCCGGTGAGC | 21 | SauCas9 | 981 |
| ADORA2A698 | GCTGAAGATGGAACTCTGCGT | 21 | SauCas9 | 982 |
| ADORA2A699 | CGTGCTTGTGTGCTGGGCCGT | 21 | SauCas9 | 983 |
| ADORA2A700 | GTGAGGACCAGGACAAAGCAG | 21 | SauCas9 | 984 |
| ADORA2A701 | TCGATGGCAATAGCCAAGAGG | 21 | SauCas9 | 985 |
| ADORA2A702 | CATCGACAGATACATCGCCAT | 21 | SauCas9 | 986 |
| ADORA2A703 | GTACACCGAGGAGCCCATGGC | 21 | SauCas9 | 987 |
| ADORA2A704 | GCTCCACCATGATGTACACCG | 21 | SauCas9 | 988 |
| ADORA2A705 | AAGCCAGTGCTGATGGTGATG | 21 | SauCas9 | 989 |
| ADORA2A706 | CACCGCGATGTCAGCCGCCGC | 21 | SauCas9 | 990 |
| ADORA2A707 | AGGCTGAAGATGGAACTCTGC | 21 | SauCas9 | 991 |
| ADORA2A708 | GCCGCCGCCAGAGATACCACG | 21 | SauCas9 | 992 |
| ADORA2A709 | AGCTCCACCATGATGTACACC | 21 | SauCas9 | 993 |
| ADORA2A710 | AGGCAGCCATGGCAGGCAGCG | 21 | SauCas9 | 994 |
| ADORA2A711 | CCTGGCTCACCGGAGTGGAAT | 21 | SauCas9 | 995 |
| ADORA2A712 | CCAGCTCCACCATGATGTACA | 21 | SauCas9 | 996 |
| ADORA2A713 | ACCAGGACAAAGCAGGCGAAG | 21 | SauCas9 | 997 |
| ADORA2A714 | CCTGGGTAACGTGCTTGTGTG | 21 | SauCas9 | 998 |
| ADORA2A715 | AGGACCAGGACAAAGCAGGCG | 21 | SauCas9 | 999 |
| ADORA2A716 | TCAGCCGCCGCCAGAGATACC | 21 | SauCas9 | 1000 |
| ADORA2A717 | GGCTCCTCGGTGTACATCATG | 21 | SauCas9 | 1001 |
| ADORA2A718 | CTGGCGGCGGCTGACATCGCG | 21 | SauCas9 | 1002 |
| ADORA2A719 | GATGGAACTCTGCGTGAGGAC | 21 | SauCas9 | 1003 |
| ADORA2A720 | GCTCCTCGGTGTACATCATGG | 21 | SauCas9 | 1004 |
| ADORA2A721 | TGTACACCGAGGAGCCCATGG | 21 | SauCas9 | 1005 |
| ADORA2A722 | GCCATTGCTGTGCTGGCCATC | 21 | SauCas9 | 1006 |
| ADORA2A723 | CAATAGCCAAGAGGCTGAAGA | 21 | SauCas9 | 1007 |
| ADORA2A724 | ATGGTGATGGCGAATGGGATG | 21 | SauCas9 | 1008 |
| ADORA2A725 | ATGTACACCGAGGAGCCCATG | 21 | SauCas9 | 1009 |
| ADORA2A726 | GTGTGGATCAACAGCAACCTG | 21 | SauCas9 | 1010 |
| ADORA2A727 | TGCTTTGTCCTGGTCCTCACG | 21 | SauCas9 | 1011 |
| ADORA2A728 | GTAACCCCTGGCTCACCGGAG | 21 | SauCas9 | 1012 |
| ADORA2A729 | CCAGCACACAAGCACGTTACC | 21 | SauCas9 | 1013 |
| ADORA2A730 | TATCTGTCGATGGCAATAGCC | 21 | SauCas9 | 1014 |
| ADORA2A731 | GCAATAGCCAAGAGGCTGAAG | 21 | SauCas9 | 1015 |
| ADORA2A732 | AGTGCTGATGGTGATGGCGAA | 21 | SauCas9 | 1016 |
| ADORA2A733 | ACACCGAGGAGCCCATGGCGG | 21 | SauCas9 | 1017 |
| ADORA2A734 | CGCCATCCGAATTCCACTCCG | 21 | SauCas9 | 1018 |
| ADORA2A4111 | TGGTGTCACTGGCGGCGGCC | 20 | AsCpf1 | 1019 |
| ADORA2A4112 | CCATCACCATCAGCACCGGG | 20 | AsCpf1 | 1020 |
| ADORA2A4113 | CCATCGGCCTGACTCCCATG | 20 | AsCpf1 | 1021 |
| ADORA2A4114 | GCTGACCGCAGTTGTTCCAA | 20 | AsCpf1 | 1022 |
| ADORA2A4115 | AGGATGTGGTCCCCATGAAC | 20 | AsCpf1 | 1023 |
| ADORA2A4116 | CCTGTGTGCTGGTGCCCCTG | 20 | AsCpf1 | 1024 |
| ADORA2A4117 | CGGATCTTCCTGGCGGCGCG | 20 | AsCpf1 | 1025 |
| ADORA2A4118 | CCCTCTGCTGGCTGCCCCTA | 20 | AsCpf1 | 1026 |
| ADORA2A4119 | TTCTGCCCCGACTGCAGCCA | 20 | AsCpf1 | 1027 |
| ADORA2A4120 | AAGGCAGCTGGCACCAGTGC | 20 | AsCpf1 | 1028 |
| ADORA2A4121 | TAAGGGCATCATTGCCATCTG | 21 | SauCas9 | 1029 |
| ADORA2A4122 | CGGCCTGACTCCCATGCTAGG | 21 | SauCas9 | 1030 |
| ADORA2A4123 | GCAGTIGTTCCAACCTAGCAT | 21 | SauCas9 | 1031 |
| ADORA2A4124 | CCGCAGTTGTTCCAACCTAGC | 21 | SauCas9 | 1032 |
| ADORA2A4125 | CAAGAACCACTCCCAGGGCTG | 21 | SauCas9 | 1033 |
| ADORA2A4126 | CTTGGCCCTCCCCGCAGCCCT | 21 | SauCas9 | 1034 |
| ADORA2A4127 | CACTTGGCCCTCCCCGCAGCC | 21 | SauCas9 | 1035 |
| ADORA2A4128 | GGCCAAGTGGCCTGTCTCTTT | 21 | SauCas9 | 1036 |
| ADORA2A4129 | TTCATGGGGACCACATCCTCA | 21 | SauCas9 | 1037 |
| ADORA2A4130 | TGAAGTACACCATGTAGTTCA | 21 | SauCas9 | 1038 |
| ADORA2A4131 | CTGGTGCCCCTGCTGCTCATG | 21 | SauCas9 | 1039 |
| ADORA2A4132 | GCTCATGCTGGGTGTCTATTT | 21 | SauCas9 | 1040 |
| ADORA2A4133 | CTTCAGCTGTCGTCGCGCCGC | 21 | SauCas9 | 1041 |
| ADORA2A4134 | CGCGACGACAGCTGAAGCAGA | 21 | SauCas9 | 1042 |
| ADORA2A4135 | GATGGAGAGCCAGCCTCTGCC | 21 | SauCas9 | 1043 |
| ADORA2A4136 | GCGTGGCTGCAGTCGGGGCAG | 21 | SauCas9 | 1044 |
| ADORA2A4137 | ACGATGGCCAGGTACATGAGC | 21 | SauCas9 | 1045 |
| ADORA2A4138 | CTCTCCCACACCAATTCGGTT | 21 | SauCas9 | 1046 |
| ADORA2A4139 | GATTCACAACCGAATTGGTGT | 21 | SauCas9 | 1047 |
| ADORA2A4140 | GGGATTCACAACCGAATTGGT | 21 | SauCas9 | 1048 |
| ADORA2A4141 | CGTAGATGAAGGGATTCACAA | 21 | SauCas9 | 1049 |
| ADORA2A4142 | GGATACGGTAGGCGTAGATGA | 21 | SauCas9 | 1050 |
| ADORA2A4143 | TCATCTACGCCTACCGTATCC | 21 | SauCas9 | 1051 |
| ADORA2A4144 | CGGATACGGTAGGCGTAGATG | 21 | SauCas9 | 1052 |
| ADORA2A4145 | GCGGAAGGTCTGGCGGAACTC | 21 | SauCas9 | 1053 |
| ADORA2A4146 | AATGATCTTGCGGAAGGTCTG | 21 | SauCas9 | 1054 |
| ADORA2A4147 | GACGTGGCTGCGAATGATCTT | 21 | SauCas9 | 1055 |
| ADORA2A4148 | TTGCTGCCTCAGGACGTGGCT | 21 | SauCas9 | 1056 |
| ADORA2A4149 | CAAGGCAGCTGGCACCAGTGC | 21 | SauCas9 | 1057 |
| ADORA2A4150 | CGGGCACTGGTGCCAGCTGCC | 21 | SauCas9 | 1058 |
| ADORA2A4151 | CTTGGCAGCTCATGGCAGIGA | 21 | SauCas9 | 1059 |
| ADORA2A4152 | CCGTCTCAACGGCCACCCGCC | 21 | SauCas9 | 1060 |
| ADORA2A4153 | CACACTCCTGGCGGGTGGCCG | 21 | SauCas9 | 1061 |
| ADORA2A4154 | TGCCGTTGGCCCACACTCCTG | 21 | SauCas9 | 1062 |
| ADORA2A4155 | CCATTGGGCCTCCGCTCAGGG | 21 | SauCas9 | 1063 |
| ADORA2A4156 | CATAGCCATTGGGCCTCCGCT | 21 | SauCas9 | 1064 |
| ADORA2A4157 | AATGGCTATGCCCTGGGGCTG | 21 | SauCas9 | 1065 |
| ADORA2A4158 | ATGCCCTGGGGCTGGTGAGTG | 21 | SauCas9 | 1066 |
| ADORA2A4159 | GCCCTGGGGCTGGTGAGTGGA | 21 | SauCas9 | 1067 |
| ADORA2A4160 | TGGIGAGTGGAGGGAGTGCCC | 21 | SauCas9 | 1068 |
| ADORA2A4161 | GAGGGAGTGCCCAAGAGTCCC | 21 | SauCas9 | 1069 |
| ADORA2A4162 | AGGGAGTGCCCAAGAGTCCCA | 21 | SauCas9 | 1070 |
| ADORA2A4163 | GTCTGGGAGGCCCGTGTTCCC | 21 | SauCas9 | 1071 |
| ADORA2A4164 | CATGGCTAAGGAGCTCCACGT | 21 | SauCas9 | 1072 |
| ADORA2A4165 | GAGCTCCTTAGCCATGAGCTC | 21 | SauCas9 | 1073 |
| ADORA2A4166 | GCTCCTTAGCCATGAGCTCAA | 21 | SauCas9 | 1074 |
| ADORA2A4167 | GGCCTAGATGACCCCCTGGCC | 21 | SauCas9 | 1075 |
| ADORA2A4168 | CCCCCTGGCCCAGGATGGAGC | 21 | SauCas9 | 1076 |
| ADORA2A4169 | CTCCTGCTCCATCCTGGGCCA | 21 | SauCas9 | 1077 |
| ADORA2A4416 | CCGTGATGTACACCGAGGAG | 20 | AsCpf1 RR | 1078 |
| ADORA2A4417 | CTTTGCCATCACCATCAGCA | 20 | AsCpf1 RR | 1079 |
| ADORA2A4418 | TTTGCCATCACCATCAGCAC | 20 | AsCpf1 RR | 1080 |
| ADORA2A4419 | TTGCCTGCTTCGTCCTGGTC | 20 | AsCpf1 RR | 1081 |
| ADORA2A4420 | TCCTGGTCCTCACGCAGAGC | 20 | AsCpf1 RR | 1082 |
| ADORA2A4421 | TCTTCAGTCTCCTGGCCATC | 20 | AsCpf1 RR | 1083 |
| ADORA2A4422 | GTCTCCTGGCCATCGCCATT | 20 | AsCpf1 RR | 1084 |
| ADORA2A4423 | ACCTAGCATGGGAGTCAGGC | 20 | AsCpf1 RR | 1085 |
| ADORA2A4424 | AACCTAGCATGGGAGTCAGG | 20 | AsCpf1 RR | 1086 |
| ADORA2A4425 | ATGCTAGGTTGGAACAACTG | 20 | AsCpf1 RR | 1087 |
| ADORA2A4426 | GCAGCCCTGGGAGTGGTTCT | 20 | AsCpf1 RR | 1088 |
| ADORA2A4427 | CGCAGCCCTGGGAGTGGTTC | 20 | AsCpf1 RR | 1089 |
| ADORA2A4428 | AGGGCTGCGGGGAGGGCCAA | 20 | AsCpf1 RR | 1090 |
| ADORA2A4429 | TGGGGACCACATCCTCAAAG | 20 | AsCpf1 RR | 1091 |
| ADORA2A4430 | CATGAACTACATGGTGTACT | 20 | AsCpf1 RR | 1092 |
| ADORA2A4431 | ATGAACTACATGGTGTACTT | 20 | AsCpf1 RR | 1093 |
| ADORA2A4432 | ACTTCTTTGCCTGTGTGCTG | 20 | AsCpf1 RR | 1094 |
| ADORA2A4433 | TGCTGCTCATGCTGGGTGTC | 20 | AsCpf1 RR | 1095 |
| ADORA2A4434 | CAAATAGACACCCAGCATGA | 20 | AsCpf1 RR | 1096 |
| ADORA2A4435 | GCTGTCGTCGCGCCGCCAGG | 20 | AsCpf1 RR | 1097 |
| ADORA2A4436 | TGGCGGCGCGACGACAGCTG | 20 | AsCpf1 RR | 1098 |
| ADORA2A4437 | TCTGCTTCAGCTGTCGTCGC | 20 | AsCpf1 RR | 1099 |
| ADORA2A4438 | GGCAGAGGCTGGCTCTCCAT | 20 | AsCpf1 RR | 1100 |
| ADORA2A4439 | CGGCAGAGGCTGGCTCTCCA | 20 | AsCpf1 RR | 1101 |
| ADORA2A4440 | CCGGCAGAGGCTGGCTCTCC | 20 | AsCpf1 RR | 1102 |
| ADORA2A4441 | CACTGCAGAAGGAGGTCCAT | 20 | AsCpf1 RR | 1103 |
| ADORA2A4442 | TGCTGCCAAGTCACTGGCCA | 20 | AsCpf1 RR | 1104 |
| ADORA2A4443 | ACAATGATGGCCAGTGACTT | 20 | AsCpf1 RR | 1105 |
| ADORA2A4444 | TACACATCATCAACTGCTTC | 20 | AsCpf1 RR | 1106 |
| ADORA2A4445 | CTTTCTTCTGCCCCGACTGC | 20 | AsCpf1 RR | 1107 |
| ADORA2A4446 | GACTGCAGCCACGCCCCTCT | 20 | AsCpf1 RR | 1108 |
| ADORA2A4447 | TCTCTGGCTCATGTACCTGG | 20 | AsCpf1 RR | 1109 |
| ADORA2A4448 | CAACCGAATTGGTGTGGGAG | 20 | AsCpf1 RR | 1110 |
| ADORA2A4449 | ACACCAATTCGGTTGTGAAT | 20 | AsCpf1 RR | 1111 |
| ADORA2A4450 | GTTGTGAATCCCTTCATCTA | 20 | AsCpf1 RR | 1112 |
| ADORA2A4451 | TTCATCTACGCCTACCGTAT | 20 | AsCpf1 RR | 1113 |
| ADORA2A4452 | TCTACGCCTACCGTATCCGC | 20 | AsCpf1 RR | 1114 |
| ADORA2A4453 | CGAGTTCCGCCAGACCTTCC | 20 | AsCpf1 RR | 1115 |
| ADORA2A4454 | GCCAGACCTTCCGCAAGATC | 20 | AsCpf1 RR | 1116 |
| ADORA2A4455 | CCAGACCTTCCGCAAGATCA | 20 | AsCpf1 RR | 1117 |
| ADORA2A4456 | GCAAGATCATTCGCAGCCAC | 20 | AsCpf1 RR | 1118 |
| ADORA2A4457 | CAAGATCATTCGCAGCCACG | 20 | AsCpf1 RR | 1119 |
| ADORA2A4458 | CAGCCACGTCCTGAGGCAGC | 20 | AsCpf1 RR | 1120 |
| ADORA2A4459 | AGGCAGCTGGCACCAGTGCC | 20 | AsCpf1 RR | 1121 |
| ADORA2A4460 | TCACTGCCATGAGCTGCCAA | 20 | AsCpf1 RR | 1122 |
| ADORA2A4461 | TCTCAACGGCCACCCGCCAG | 20 | AsCpf1 RR | 1123 |
| ADORA2A4462 | CTCAGGGTGGGGAGCACTGC | 20 | AsCpf1 RR | 1124 |
| ADORA2A4463 | CACCCTGAGCGGAGGCCCAA | 20 | AsCpf1 RR | 1125 |
| ADORA2A4464 | ACCCTGAGCGGAGGCCCAAT | 20 | AsCpf1 RR | 1126 |
| ADORA2A4465 | AGGGCATAGCCATTGGGCCT | 20 | AsCpf1 RR | 1127 |
| ADORA2A4466 | CTCACCAGCCCCAGGGCATA | 20 | AsCpf1 RR | 1128 |
| ADORA2A4467 | TCCACTCACCAGCCCCAGGG | 20 | AsCpf1 RR | 1129 |
| ADORA2A4468 | TGGGACTCTTGGGCACTCCC | 20 | AsCpf1 RR | 1130 |
| ADORA2A4469 | CTGGGACTCTTGGGCACTCC | 20 | AsCpf1 RR | 1131 |
| ADORA2A4470 | CCTGGGACTCTTGGGCACTC | 20 | AsCpf1 RR | 1132 |
| ADORA2A4471 | AGGGGAACACGGGCCTCCCA | 20 | AsCpf1 RR | 1133 |
| ADORA2A4472 | CGTCTGGGAGGCCCGTGTTC | 20 | AsCpf1 RR | 1134 |
| ADORA2A4473 | AGACGTGGAGCTCCTTAGCC | 20 | AsCpf1 RR | 1135 |
| ADORA2A4474 | TTGAGCTCATGGCTAAGGAG | 20 | AsCpf1 RR | 1136 |
| ADORA2A4475 | CTGGCCTAGATGACCCCCTG | 20 | AsCpf1 RR | 1137 |
| ADORA2A4476 | TGGCCTAGATGACCCCCTGG | 20 | AsCpf1 RR | 1138 |
| ADORA2A4477 | TCCTGGGCCAGGGGGTCATC | 20 | AsCpf1 RR | 1139 |
| ADORA2A4478 | CTGGCCCAGGATGGAGCAGG | 20 | AsCpf1 RR | 1140 |
| ADORA2A4479 | TGGCCCAGGATGGAGCAGGA | 20 | AsCpf1 RR | 1141 |
| ADORA2A4480 | CGCGAGTTCCGCCAGACCIT | 20 | AsCpf1 RVR | 1142 |
| ADORA2A4481 | CCCTGGGGCTGGTGAGTGGA | 20 | AsCpf1RVR | 1143 |
| ADORA2A4482 | CCATCGGCCTGACTCCCATGC | 21 | Cas12a | 1174 |

It will be understood that the exemplary gRNAs disclosed herein are provided to illustrate non-limiting embodiments embraced by the present disclosure. Additional suitable gRNA sequences will be apparent to the skilled artisan based on the present disclosure, and the disclosure is not limited in this respect.

### Nucleases

Any nuclease that causes a break within an endogenous coding sequence of an essential gene of the cell can be used in the methods of the present disclosure. In some embodiments the nuclease is a DNA nuclease. In some embodiments the nuclease causes a single-strand break (SSB) within an endogenous coding sequence of an essential gene of the cell, e.g., in a “prime editing” system. In some embodiments the nuclease causes a double-strand break (DSB) within an endogenous coding sequence of an essential gene of the cell. In some embodiments the double-strand break is caused by a single nuclease. In some embodiments the double-strand break is caused by two nucleases that each cause a single-strand break on opposing strands, e.g., a dual “nickase” system. In some embodiments the nuclease is a CRISPR/Cas nuclease and the method further comprises contacting the cell with one or more guide molecules for the CRISPR/Cas nuclease. Exemplary CRISPR/Cas nucleases and guide molecules are described in more detail herein. It is to be understood that the nuclease (including a nickase) is not limited in any manner and can also be a zinc finger nuclease (ZFN), a transcription activator-like effector nuclease (TALEN), a meganuclease, or other nuclease known in the art (or a combination thereof). Methods for designing zinc finger nucleases (ZFNs) are well known in the art, e.g., see Urnov et al., Nature Reviews Genetics 2010; 11:636-640 and Paschon et al., Nat. Commun. 2019; 10(1):1133 and references cited therein. Methods for designing transcription activator-like effector nucleases (TALENs) are well known in the art, e.g., see Joung and Sander, Nat. Rev. Mol. Cell Biol. 2013; 14(1):49-55 and references cited therein. Methods for designing meganucleases are also well known in the art, e.g., see Silva et al., Curr. Gene Ther. 2011; 11(1): 11-27 and Redel and Prather, Toxicol. Pathol. 2016; 44(3):428-433.

In some embodiments, a nuclease suitable for methods described herein can have an editing efficiency that is greater than about 50%. In some embodiments, a nuclease suitable for methods described herein can have an editing efficiency that is greater than about 55%. In some embodiments, a nuclease suitable for methods described herein can have an editing efficiency that is greater than about 60%. In some embodiments, a nuclease suitable for methods described herein can have an editing efficiency that is greater than about 65%. In some embodiments, a nuclease suitable for methods described herein can have an editing efficiency that is greater than about 70%. In some embodiments, a nuclease suitable for methods described herein can have an editing efficiency that is greater than about 75%. In some embodiments, a nuclease suitable for methods described herein can have an editing efficiency that is greater than about 80%. In some embodiments, a nuclease suitable for methods described herein can have an editing efficiency that is greater than about 85%. In some embodiments, a nuclease suitable for methods described herein can have an editing efficiency that is greater than about 90%. In some embodiments, a nuclease suitable for methods described herein can have an editing efficiency that is greater than about 95%. In some embodiments, a nuclease suitable for methods described herein can have an editing efficiency that is greater than about 96%. In some embodiments, a nuclease suitable for methods described herein can have an editing efficiency that is greater than about 97%. In some embodiments, a nuclease suitable for methods described herein can have an editing efficiency that is greater than about 98%. In some embodiments, a nuclease suitable for methods described herein can have an editing efficiency that is greater than about 99%.

In general, the nuclease can be delivered to the cell as a protein or a nucleic acid encoding the protein, e.g., a DNA molecule or mRNA molecule. The protein or nucleic acid can be combined with other delivery agents, e.g., lipids or polymers in a lipid or polymer nanoparticle and targeting agents such as antibodies or other binding agents with specificity for the cell. The DNA molecule can be a nucleic acid vector, such as a viral genome or circular double-stranded DNA, e.g., a plasmid. Nucleic acid vectors encoding a nuclease can include other coding or non-coding elements. For example, a nuclease can be delivered as part of a viral genome (e.g., in an AAV, adenoviral or lentiviral genome) that includes certain genomic backbone elements (e.g., inverted terminal repeats, in the case of an AAV genome).

A CRISPR/Cas nuclease can be delivered to the cell as a protein or a nucleic acid encoding the protein, e.g., a DNA molecule or mRNA molecule. The guide molecule can be delivered as an RNA molecule or encoded by a DNA molecule. A CRISPR/Cas nuclease can also be delivered with a guide molecule as a ribonucleoprotein (RNP) and introduced into the cell via nucleofection (electroporation).

### RNA-Guided Nucleases

RNA-guided nucleases according to the present disclosure include, but are not limited to, naturally-occurring Class 2 CRISPR nucleases such as Cas9, and Cpf1 (Cas12a), as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif.” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer. Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389 Sep. 12, 2013 (“Ran”)), or that that do not cut at all.

### Cas9

Crystal structures have been determined for S. pyogenes Cas9 (Jinek et al., Science 343(6176), 1247997, 2014 (“Jinek 2014”), and for S. aureus Cas9 in complex with a unimolecular guide RNA and a target DNA (Nishimasu 2014; Anders et al., Nature. 2014 Sep. 25; 513(7519):569-73 (“Anders 2014”); and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat: antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

### Cpf1

The crystal structure of Acidaminococcus sp. Cpf1 in complex with crRNA and a dsDNA target including a TTTN PAM sequence has been solved by Yamano et al. (Cell. 2016 May 5; 165(4): 949-962 (“Yamano”), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

### Nuclease Variants

The RNA-guided nucleases described herein have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran & Hsu, et al., (Cell 154(6), 1380-1389 Sep. 12, 2013), and Yamano, et al. (Cell. 2016 May 5; 165(4): 949-962); as well as in WO 2016/073990 by Cotta-Ramusino, the entire contents of each of which are incorporated herein by reference. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain or of a Cas9 HNH domain results in a nickase. Exemplary nickase variants include Cas9 D10A and Cas9 H840A (numbering scheme according to SpCas9 wild-type sequence). Additional suitable nickase variants, including Cas12a variants, will be apparent to the skilled artisan based on the present disclosure and the knowledge in the art. The present disclosure is not limited in this respect. In some embodiments a nickase may be fused to a reverse transcriptase to produce a prime editor (PE), e.g., as described in Anzalone et al., Nature 2019; 576:149-157, the entire contents of which are incorporated herein by reference.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5); and S. aureus (Kleinstiver et al., Nat Biotechnol. 2015 December; 33(12): 1293-1298). Kleinstiver et al. have also described modifications that improve the targeting fidelity of Cas9 (Nature, 2016 Jan. 28; 529, 490-495). Each of these references is incorporated by reference herein.

RNA-guided nucleases have been split into two or more parts, as described by Zetsche et al. (Nat Biotechnol. 2015 February; 33(2):139-42, incorporated by reference), and by Fine et al. (Sci Rep. 2015 Jul. 1; 5:10777, incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference herein.

RNA-guided nucleases also optionally include a tag, such as, but not limited to, a nuclear localization signal, to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

Exemplary suitable nuclease variants include, but are not limited to, AsCpf1 variants comprising an M537R substitution, an H800A substitution, and/or an F870L substitution, or any combination thereof (numbering scheme according to AsCpf1 wild-type sequence). In some embodiments, an ASCpf1 variant comprises an M537R substitution, an H800A substitution, and an F870L substitution. Other suitable modifications of the AsCpf1 amino acid sequence are known to those of ordinary skill in the art. Some exemplary sequences of wild-type AsCpf1 and AsCpf1 variants are provided below:

His-AsCpf1-sNLS-sNLS H800A amino acid sequence
(SEQ ID NO: 1144):
MGHHHHHHGSTQFEGFTNLYQVSKTLRFELIPQGKTLKHIQEQGF
IEEDKARNDHYKELKPIIDRIYKTYADQCLQLVQLDWENLSAAID
SYRKEKTEETRNALIEE
QATYRNAIHDYFIGRTDNLTDAINKRHAEIYKGLFKAELFNGKVL
KQLGTVTTTEHENALLRSFDKFTTYFSGFYENRKNVFSAEDISTA
IPHRIVQDNFPKFKENCHIFTRLITAVPSLREHFENVKKAIGIFV
STSIEEVFSFPFYNQLLTQTQIDLYNQLLGGISREAGTEKIKGLN
EVLNLAIQKNDETAHIIASLPHRFIPLFKQILSDRNTLSFILEEF
KSDEEVIQSFCKYKTLLRNENVLETAEALFNELNSIDLTHIFISH
KKLETISSALCDHWDTLRNALYERRISELTGKITKSAKEKVQRSL
KHEDINLQEIISAAGKELSEAFKQKISEILSHAHAALDQPLPTTL
KKQEEKEILKSQLDSLLGLYHLLDWFAVDESNEVDPEFSARLTGI
KLEMEPSLSFYNKARNYATKKPYSVEKFKLNFQMPTLASGWDVNK
EKNNGAILFVKNGLYYLGIMPKQKGRYKALSFEPTEKTSEGFDKM
YYDYFPDAAKMIPKCSTQLKAVTAHFQTHITPILLSNNFIEPLEI
TKEIYDLNNPEKEPKKFQTAYAKKIGDQKGYREALCKWIDFTRDE
LSKYTKTISIDLSSLRPSSQYKDLGEYYAELNPLLYHISFQRIAE
KEIMDAVETGKLYLFQIYNKDFAKGHHGKPNLHILYWIGLFSPEN
LAKISIKLNGQAELFYRPKSRMKRMAARLGEKMLNKKLKDQKTPI
PDTLYQELYDYVNHRLSHDLSDEARALLPNVITKEVSHEIIKDRR
FTSDKFFFHVPIILNYQAANSPSKFNQRVNAYLKEHPETPIIGID
RGERNLIYITVIDSIGKILEQRSLNTIQQFDYQKKLDNREKERVA
ARQAWSVVGTIKDLKQGYLSQVIHEIVDLMIHYQAVVVLENLNFG
FKSKRIGIAEKAVYQQFEKMLIDKLNCLVLKDYPAEKVGGVLNPY
QLTDQFTSFAKMGTQSGFLFYVPAPYTSKIDPLIGFVDPFVWKTI
KNHESRKHFLEGFDFLHYDVKTGDFILHFKMNRNLSFQRGLPGFM
PAWDIVFEKNETQFDAKGIPFIAGKRIVPVIENHRFTGRYRDLYP
ANELIALLEEKGIVERDGSNILPKLLENDDSHAIDTMVALIRSVL
QMRNSNAATGEDYINSPVRDLNGVCFDSRFQNPEWPMDADANGAY
HIALKGQLLLNHLKESKDLKLQNGISNQDWLAYIQELRNGSPKKK
RKVGSPKKKRKV
Cpf1 variant 1 amino acid sequence
(SEQ ID NO: 1145):
MTQFEGFTNLYQVSKTLRFELIPQGKILKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKILLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLIGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRIGIAEKAVYQQFEKMLIDKLNCLVLKDYPAEK
VGGVLNPYQLTDQFTSFAKMGTQSGFLFYVPAPYTSKIDPLIGFV
DPFVWKIIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 2 amino acid sequence
(SEQ ID NO: 1146):
MTQFEGFTNLYQVSKILRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRIDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNI
LSFILEEFKSDEEVIQSFCKYKILLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKIGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
IGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLIGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 3 amino acid sequence
(SEQ ID NO: 1147):
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKILLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
IGLFSPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRIGIAEKAVYQQFEKMLIDKLNCLVLKDYPAEK
VGGVLNPYQLTDQFTSFAKMGTQSGFLFYVPAPYTSKIDPLIGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 4 amino acid sequence
(SEQ ID NO: 1148):
MTQFEGFTNLYQVSKILRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRIDNLTDAINKRHAEIYKGLFKA
ELFNGKVLKQLGTVTTTEHENALLRSFDKFTTYFSGFYENRKNVE
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLEKQILSDRNI
LSFILEEFKSDEEVIQSFCKYKILLRNENVLETAEALFNELNSID
LTHIFISHKKLETISSALCDHWDTLRNALYERRISELIGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
IGLFSPENLAKISIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRIGIAEKAVYQQFEKMLIDKLNCLVLKDYPAEK
VGGVLNPYQLTDQFTSFAKMGTQSGFLFYVPAPYTSKIDPLIGFV
DPFVWKTIKNHESRKHFLEGFDFLHYDVKIGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKV
Cpf1 variant 5 amino acid sequence
(SEQ ID NO: 1149):
MTQFEGFTNLYQVSKILRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNI
LSFILEEFKSDEEVIQSFCKYKILLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLIGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKIGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKV
Cpf1 variant 6 amino acid sequence
(SEQ ID NO: 1150):
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRIDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNI
LSFILEEFKSDEEVIQSFCKYKILLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRIGIAEKAVYQQFEKMLIDKLNCLVLKDYPAEK
VGGVLNPYQLTDQFTSFAKMGTQSGFLFYVPAPYTSKIDPLIGFV
DPFVWKTIKNHESRKHFLEGFDFLHYDVKIGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 7 amino acid sequence
(SEQ ID NO: 1151):
MGRDPGKPIPNPLLGLDSTAPKKKRKVGIHGVPAATQFEGFTNLY
QVSKTLRFELIPQGKTLKHIQEQGFIEEDKARNDHYKELKPIIDR
IYKTYADQCLQLVQLDWENLSAAIDSYRKEKTEETRNALIEEQAT
YRNAIHDYFIGRIDNLTDAINKRHAEIYKGLFKAELFNGKVLKQL
GTVTTTEHENALLRSFDKFTTYFSGFYENRKNVFSAEDISTAIPH
RIVQDNFPKFKENCHIFTRLITAVPSLREHFENVKKAIGIFVSTS
IEEVFSFPFYNQLLTQTQIDLYNQLLGGISREAGTEKIKGLNEVL
NLAIQKNDETAHIIASLPHRFIPLFKQILSDRNTLSFILEEFKSD
EEVIQSFCKYKTLLRNENVLETAEALFNELNSIDLTHIFISHKKL
ETISSALCDHWDTLRNALYERRISELTGKITKSAKEKVQRSLKHE
DINLQEIISAAGKELSEAFKQKTSEILSHAHAALDQPLPTTLKKQ
EEKEILKSQLDSLLGLYHLLDWFAVDESNEVDPEFSARLIGIKLE
MEPSLSFYNKARNYATKKPYSVEKFKLNFQMPTLASGWDVNKEKN
NGAILFVKNGLYYLGIMPKQKGRYKALSFEPTEKTSEGFDKMYYD
YFPDAAKMIPKCSTQLKAVTAHFQTHTTPILLSNNFIEPLEITKE
IYDLNNPEKEPKKFQTAYAKKIGDQKGYREALCKWIDFTRDFLSK
YTKTISIDLSSLRPSSQYKDLGEYYAELNPLLYHISFQRIAEKEI
MDAVETGKLYLFQIYNKDFAKGHHGKPNLHTLYWTGLFSPENLAK
TSIKLNGQAELFYRPKSRMKRMAHRLGEKMLNKKLKDQKTPIPDT
LYQELYDYVNHRLSHDLSDEARALLPNVITKEVSHEIIKDRRFTS
DKFFFHVPITLNYQAANSPSKFNQRVNAYLKEHPETPIIGIDRGE
RNLIYITVIDSTGKILEQRSLNTIQQFDYQKKLDNREKERVAARQ
AWSVVGTIKDLKQGYLSQVIHEIVDLMIHYQAVVVLENLNFGFKS
KRIGIAEKAVYQQFEKMLIDKLNCLVLKDYPAEKVGGVLNPYQLT
DQFTSFAKMGTQSGFLFYVPAPYTSKIDPLTGFVDPFVWKTIKNH
ESRKHFLEGFDFLHYDVKIGDFILHFKMNRNLSFQRGLPGFMPAW
DIVFEKNETQFDAKGTPFIAGKRIVPVIENHRFTGRYRDLYPANE
LIALLEEKGIVERDGSNILPKLLENDDSHAIDTMVALIRSVLQMR
NSNAATGEDYINSPVRDLNGVCFDSRFQNPEWPMDADANGAYHIA
LKGQLLLNHLKESKDLKLQNGISNQDWLAYIQELRNPKKKRKVKL
AAALEHHHHHH
Exemplary AsCpf1 wild-type amino acid sequence
(SEQ ID NO: 1152):
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRIDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKILLRNENVLETAEALFNELNSID
LTHIFISHKKLETISSALCDHWDTLRNALYERRISELIGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKITSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRIGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN

Additional suitable nucleases and nuclease variants will be apparent to the skilled artisan based on the present disclosure in view of the knowledge in the art. Exemplary suitable nucleases may include, but are not limited to, those provided in Table 2 herein.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

As an example, the nucleic acid sequence for Cpf1 variant 4 is set forth below as SEQ ID NO: 1177

ATGACCCAGTTTGAAGGITTCACCAATCTGTATCAGGITAGCAAAACCCTGCGTTTTGAACT
GATTCCGCAGGGTAAAACCCTGAAACATATTCAAGAACAGGGCTTCATCGAAGAGGATAAAG
CACGTAACGATCACTACAAAGAACTGAAACCGATTATCGACCGCATCTATAAAACCTATGCA
GATCAGTGTCTGCAGCTGGTTCAGCTGGATTGGGAAAATCTGAGCGCAGCAATTGATAGTTA
TCGCAAAGAAAAAACCGAAGAAACCCGTAATGCACTGATTGAAGAACAGGCAACCTATCGTA
ATGCCATCCATGATTATTTCATTGGTCGTACCGATAATCTGACCGATGCAATTAACAAACGT
CACGCCGAAATCTATAAAGGCCTGTTTAAAGCCGAACTGITTAATGGCAAAGTTCTGAAACA
GCTGGGCACCGTTACCACCACCGAACATGAAAATGCACTGCTGCGTAGCTTTGATAAATTCA
CCACCTATTTCAGCGGCTTTTATGAGAATCGCAAAAACGTGTTTAGCGCAGAAGATATTAGC
ACCGCAATTCCGCATCGTATTGTGCAGGATAATTTCCCGAAATTCAAAGAGAACTGCCACAT
TTTTACCCGTCTGATTACCGCAGTTCCGAGCCTGCGTGAACATTTTGAAAACGTTAAAAAAG
CCATCGGCATCTTTGTTAGCACCAGCATTGAAGAAGTTTTTAGCTTCCCGITTTACAATCAG
CTGCTGACCCAGACCCAGATTGATCTGTATAACCAACTGCTGGGTGGTATTAGCCGTGAAGC
AGGCACCGAAAAAATCAAAGGICTGAATGAAGTGCTGAATCTGGCCATTCAGAAAAATGATG
AAACCGCACATATTATTGCAAGCCTGCCGCATCGTTTTATTCCGCTGTTCAAACAAATTCTG
AGCGATCGTAATACCCTGAGCTTTATTCTGGAAGAATTCAAATCCGATGAAGAGGIGATTCA
GAGCTTTTGCAAATACAAAACGCTGCTGCGCAATGAAAATGTTCTGGAAACTGCCGAAGCAC
TGTTTAACGAACTGAATAGCATTGATCTGACCCACATCTTTATCAGCCACAAAAAACTGGAA
ACCATTTCAAGCGCACTGTGTGATCATTGGGATACCCTGCGTAATGCCCTGTATGAACGTCG
TATTAGCGAACTGACCGGTAAAATTACCAAAAGCGCGAAAGAAAAAGTTCAGCGCAGTCTGA
AACATGAGGATATTAATCTGCAAGAGATTATTAGCGCAGCCGGTAAAGAACTGTCAGAAGCA
TTTAAACAGAAAACCAGCGAAATTCTGTCACATGCACATGCAGCACTGGATCAGCCGCTGCC
GACCACCCTGAAAAAACAAGAAGAAAAAGAAATCCTGAAAAGCCAGCTGGATAGCCTGCTGG
GTCTGTATCATCTGCTGGACTGGTTTGCAGTTGATGAAAGCAATGAAGTTGATCCGGAATTT
AGCGCACGTCTGACCGGCATTAAACTGGAAATGGAACCGAGCCTGAGCTTTTATAACAAAGC
CCGTAATTATGCCACCAAAAAACCGTATAGCGTCGAAAAATTCAAACTGAACTTTCAGCGTC
CGACCCTGGCAAGCGGTTGGGATGTTAATAAAGAAAAAAACAACGGTGCCATCCTGTTCGTG
AAAAATGGCCTGTATTATCTGGGTATTATGCCGAAACAGAAAGGTCGTTATAAAGCGCTGAG
CTTTGAACCGACGGAAAAAACCAGTGAAGGTTTTGATAAAATGTACTACGACTATTTTCCGG
ATGCAGCCAAAATGATTCCGAAATGTAGCACCCAGCTGAAAGCAGTTACCGCACATTTTCAG
ACCCATACCACCCCGATTCTGCTGAGCAATAACTTTATTGAACCGCTGGAAATCACCAAAGA
GATCTACGATCTGAATAACCCGGAAAAAGAGCCGAAAAAATTCCAGACCGCATATGCAAAAA
AAACCGGTGATCAGAAAGGTTATCGTGAAGCGCTGTGTAAATGGATTGATTTCACCCGTGAT
TTTCTGAGCAAATACACCAAAACCACCAGTATCGATCTGAGCAGCCTGCGTCCGAGCAGCCA
GTATAAAGATCTGGGCGAATATTATGCAGAACTGAATCCGCTGCTGTATCATATTAGCTTTC
AGCGTATTGCCGAGAAAGAAATCATGGACGCAGTTGAAACCGGTAAACTGTACCTGTTCCAG
ATCTACAATAAAGATTTTGCCAAAGGCCATCATGGCAAACCGAATCTGCATACCCTGTATTG
GACCGGTCTGTTTAGCCCTGAAAATCTGGCAAAAACCTCGATTAAACTGAATGGTCAGGCGG
AACTGTTTTATCGTCCGAAAAGCCGTATGAAACGTATGGCAGCTCGTCTGGGTGAAAAAATG
CTGAACAAAAAACTGAAAGACCAGAAAACCCCGATCCCGGATACACTGTATCAAGAACTGTA
TGATTATGTGAACCATCGTCTGAGCCATGATCTGAGTGATGAAGCACGTGCCCTGCTGCCGA
ATGTTATTACCAAAGAAGTTAGCCACGAGATCATTAAAGATCGTCGTTTTACCAGCGACAAA
TTCCTGTTTCATGTGCCGATTACCCTGAATTATCAGGCAGCAAATAGCCCGAGCAAATTTAA
CCAGCGTGTTAATGCATATCTGAAAGAACATCCAGAAACGCCGATTATTGGTATTGATCGTG
GTGAACGTAACCTGATTTATATCACCGTTATTGATAGCACCGGCAAAATCCTGGAACAGCGT
AGCCTGAATACCATTCAGCAGITTGATTACCAGAAAAAACTGGATAATCGCGAGAAAGAACG
TGTTGCAGCACGTCAGGCATGGTCAGTTGTTGGTACAATTAAAGACCTGAAACAGGGTTATC
TGAGCCAGGITATTCATGAAATTGTGGATCTGATGATTCACTATCAGGCCGTIGTTGIGCTG
GAAAACCTGAATTTTGGCTTTAAAAGCAAACGTACCGGCATTGCAGAAAAAGCAGTTTATCA
GCAGTTCGAGAAAATGCTGATTGACAAACTGAATTGCCTGGTGCTGAAAGATTATCCGGCTG
AAAAAGTTGGTGGTGTTCTGAATCCGTATCAGCTGACCGATCAGTTTACCAGCTTTGCAAAA
ATGGGCACCCAGAGCGGATTTCTGTTTTATGTTCCGGCACCGTATACGAGCAAAATTGATCC
GCTGACCGGTTTTGTTGATCCGTTTGTTTGGAAAACCATCAAAAACCATGAAAGCCGCAAAC
ATTTTCTGGAAGGTTTCGATTTTCTGCATTACGACGTTAAAACGGGTGATTTCATCCTGCAC
TTTAAAATGAATCGCAATCTGAGTTTTCAGCGTGGCCTGCCTGGTITTATGCCTGCATGGGA
TATTGTGTTTGAGAAAAACGAAACACAGTTCGATGCAAAAGGCACCCCGTTTATTGCAGGTA
AACGTATTGTTCCGGTGATTGAAAATCATCGTTTCACCGGTCGTTATCGCGATCTGTATCCG
GCAAATGAACTGATCGCACTGCTGGAAGAGAAAGGTATTGTTTTTCGTGATGGCTCAAACAT
TCTGCCGAAACTGCTGGAAAATGATGATAGCCATGCAATTGATACCATGGTTGCACTGATTC
GTAGCGTTCTGCAGATGCGTAATAGCAATGCAGCAACCGGTGAAGATTACATTAATAGTCCG
GTTCGTGATCTGAATGGTGTTTGTTTTGATAGCCGTTTTCAGAATCCGGAATGGCCGATGGA
TGCAGATGCAAATGGIGCATATCATATTGCACTGAAAGGACAGCTGCTGCTGAACCACCTGA
AAGAAAGCAAAGATCTGAAACTGCAAAACGGCATTAGCAATCAGGATTGGCTGGCATATATC
CAAGAACTGCGTAACGGTCGTAGCAGTGATGATGAAGCAACCGCAGATAGCCAGCATGCAGC
ACCGCCTAAAAAGAAACGTAAAGTT

### Activin

The TGF-β superfamily consists of more than 45 members including activins, inhibins, myostatin, bone morphogenetic proteins (BMPs), growth and differentiation factors (GDFs) and nodal (see, e.g., Morianos et al., Journal of Autoimmunity 104:102314 (2019)). Activins are found either as homodimers or heterodimers of βA or/and βB subunits linked with disulfide bonds. There are three functional isoforms of activins: activin-A (BABA), activin B (βBβB) and activin AB (βAβB) (Xia et al., J. Endocrinol. 202:1-12 (2009)). The βC and βE subunits are found in mammals and the βB subunit in Xenopus laevis. Transcripts of the βA and βB subunits are detected in nearly every tissue in the human body and exhibit increased expression in the reproductive system, while the BC and BE subunits are predominantly expressed in the liver (Woodruff, Biochem. Pharmacol. 55:953-963 (1998)). Activin-A is a cytokine of approximately 25 kDa and represents the most extensively investigated protein among the family of activins. Activin-A was initially identified as a gonadal protein that induces the biosynthesis and secretion of the follicle-stimulating hormone from the pituitary (Hedger et al., Cytokine Growth Factor Rev. 24:285-295 (2013)). It is highly conserved among vertebrates, reaching up to 95% homology between species. Activin-A regulates fundamental biologic processes, such as, haematopoiesis, embryonic development, stem cell maintenance and pluripotency, tissue repair and fibrosis (Kariyawasam et al., Clin. Exp. Allergy 41:1505-1514 (2011)). Activin, e.g., Activin A, is well known and commercially available (from, e.g., STEMCELL Technologies Inc., Cambridge, MA).

### Culture Methods

In general, an ES cell (e.g., an ES cell genetically engineered not to express one or more TGFβ receptor, e.g., TGFβRII) can be cultured to maintain pluripotency by culturing such ES cells in media that contains activin, e.g., a particular, effective level of activin (e.g., during one or more stages of culture).

In some embodiments, ES cells described herein are cultured (e.g., at one or more stages of culture) in a medium that includes activin, e.g., an elevated level of activin, to maintain pluripotency of the cells. In some embodiments, a level of one or more ES markers (e.g., SSEA-3, SSEA-4, TRA-1-60, TRA-1-81, TRA-2-49/6E, ALP, Sox2, E-cadherin, UTF-1, Oct4, Rex 1, and/or Nanog) in a sample of cells from the culture is increased relative to the corresponding level(s) in a sample of cells cultured using the same medium that does not include activin, e.g., an elevated level of activin. In some embodiments, the increased level of one or more ES marker is higher than the corresponding level(s) by at least about 10%, 20%, 30%, 40%, 50%, 60%, 70%, 80%, 90%, 100%, 150%, 200%, 250%, 300%, 350%, 400%, 450%, 500%, or more, of the corresponding level.

As used herein, an “elevated level of activin” means a higher concentration of activin than is present in a standard medium, a starting medium, a medium used at one or more stages of culture, and/or in a medium in which ES cells are cultured. In some embodiments, activin is not present in a standard and/or starting medium, a medium used at one or more other stages of culture, and/or in a medium in which ES cells are cultured, and an “elevated level” is any amount of activin. A medium can include an elevated level of activin initially (i.e., at the start of a culture), and/or medium can be supplemented with activin to achieve an elevated level of activin at a particular time or times (e.g., at one or more stages) during culturing.

In some embodiments, an elevated level of activin is an increase of at least about 10%, 20%, 30%, 40%, 50%, 60%, 70%, 80%, 90%, 100%, 150%, 200%, 250%, 300%, 350%. 400%, 450%, 500%, 550%, 600%, 650%, 700%, 750%, 800%, 850%, 900%, 950%, 1000% or more, relative to a level of activin in a standard medium, a starting medium, a medium during one or more stages of culture, and/or in a medium in which ES cells are cultured.

In some embodiments, an elevated level of activin is about 0.5 ng/mL, 1 ng/mL, 2 ng/ml, 3 ng/mL, 4 ng/ml, 5 ng/mL, 10 ng/ml, 15 ng/ml, 20 ng/mL, 25 ng/ml, 30 ng/ml, 35 ng/ml, 40 ng/ml, 45 ng/mL, 50 ng/ml, 60 ng/ml, 70 ng/ml, 80 ng/ml, 90 ng/mL, 100 ng/mL, or more, activin. In some embodiments, an elevated level of activin is about 0.5 ng/mL to about 20 ng/ml activin, about 0.5 ng/ml to about 10 ng/ml activin, about 4 ng/ml to about 10 ng/mL activin.

Cells can be cultured in a variety of cell culture media known in the art, which are modified according to the disclosure to include activin as described herein. Cell culture medium is understood by those of skill in the art to refer to a nutrient solution in which cells, such as animal or mammalian cells, are grown. A cell culture medium generally includes one or more of the following components: an energy source (e.g., a carbohydrate such as glucose); amino acids; vitamins; lipids or free fatty acids; and trace elements, e.g., inorganic compounds or naturally occurring elements in the micromolar range. Cell culture medium can also contain additional components, such as hormones and other growth factors (e.g., insulin, transferrin, epidermal growth factor, serum, and the like); signaling factors (e.g., interleukin 15 (IL-15), transforming growth factor beta (TGF-β), and the like); salts (e.g., calcium, magnesium and phosphate); buffers (e.g., HEPES); nucleosides and bases (e.g., adenosine, thymidine, hypoxanthine); antibiotics (e.g., gentamycin); and cell protective agents (e.g., a Pluronic polyol (Pluronic F68)).

Media that has been prepared or commercially available can be modified according to the present disclosure for utilization in the methods described herein. Nonlimiting examples of such media include Minimal Essential Medium (MEM, Sigma, St. Louis, Mo.); Ham's F10 Medium (Sigma); Dulbecco's Modified Eagles Medium (DMEM, Sigma); RPM I-1640 Medium (Sigma); HyClone cell culture medium (HyClone, Logan, Utah); Power CHO2 (Lonza Inc., Allendale, NJ); and chemically-defined (CD) media, which are formulated for particular cell types. In some embodiments, a culture medium is an E8 medium described in, e.g., Chen et al., Nat. Methods 8:424-429 (2011)). In some embodiments, a cell culture medium includes activin but lacks TGFβ.

Cell culture conditions (including pH, O₂, CO₂, agitation rate and temperature) suitable for ES cells are those that are known in the art, such as described in Schwartz et al., Methods Mol. Biol. 767:107-123 (2011) and Chen et al., Nat. Methods 8:424-429 (2011).

In some embodiments, cells are cultured in one or more stages, and cells can be cultured in medium having an elevated level of activin in one or more stages. For example, a culture method can include a first stage (e.g., using a medium having a reduced level of or no activin) and a second stage (e.g., using a medium having an elevated level of activin). In some embodiments, a culture method can include a first stage (e.g., using a medium having an elevated level of activin) and a second stage (e.g., using a medium having a reduced level of activin). In some embodiments, a culture method includes more than two stages, e.g., 3, 4, 5, 6, or more stages, and any stage can include medium having an elevated level of activin or a reduced level of activin. The length of culture is not limiting. For example, a culture method can be 5, 6, 7, 8, 9, 10, 11, 12, 13, 14, 15, 16, 17, 18, 19, 20, or more days. In some embodiments, a culture method includes at least two stages. For example, a first stage can include culturing cells in medium having a reduced level of activin (e.g., for about 1, 2, 3, 4, 5, 6, 7, 8, 9, 10, or more days), and a second stage can include culturing cells in medium having an elevated level of activin (e.g., for about 1, 2, 3, 4, 5, 6, 7, 8, 9, 10, or more days). In some embodiments, a first stage can include culturing cells in medium having an elevated level of activin (e.g., for about 1, 2, 3, 4, 5, 6, 7, 8, 9, 10, or more days), and a second stage can include culturing cells in medium having a reduced level of activin (e.g., for about 1, 2, 3, 4, 5, 6, 7, 8, 9, 10, or more days).

In particular methods, levels of one or more ES marker (e.g., SSEA-3, SSEA-4. TRA-1-60, TRA-1-81, TRA-2-49/6E, ALP, Sox2, E-cadherin, UTF-1, Oct4, Rex1, and/or Nanog) expressed in a sample of cells from a cell culture are monitored during one or more times (e.g., one or more stages) of cell culture, thereby allowing adjustment (e.g., increasing or decreasing the amount of activin in the culture) stopping the culture, and/or harvesting the cells from the culture.

### Methods of Characterization

Methods of characterizing cells including characterizing cellular phenotype are known to those of skill in the art. In some embodiments, one or more such methods may include, but not be limited to, for example, morphological analyses and flow cytometry. Cellular lineage and identity markers are known to those of skill in the art. One or more such markers may be combined with one or more characterization methods to determine a composition of a cell population or phenotypic identity of one or more cells. For example, in some embodiments, cells of a particular population will be characterized using flow cytometry. In some such embodiments, a sample of a population of cells will be evaluated for presence and proportion of one or more cell surface markers and/or one or more intracellular markers. As will be understood by those of skill in the art, such cell surface markers may be representative of different lineages. For example, pluripotent cells may be identified by one or more of any number of markers known to be associated with such cells, such as, for example, CD34. Further, in some embodiments, cells may be identified by markers that indicate some degree of differentiation. Such markers will be known to one of skill in the art. For example, in some embodiments, markers of differentiated cells may include those associated with differentiated hematopoietic cells such as, e.g., CD43, CD45 (differentiated hematopoietic cells). In some embodiments, markers of differentiated cells may be associated with NK cell phenotypes such as, e.g., CD56 (also known as neural cell adhesion molecule), NK cell receptor immunoglobulin gamma Fc region receptor III (FcγRIII, cluster of differentiation 16 (CD16), natural killer group-2 member A (NKG2A), natural killer group-2 member D (NKG2D), CD69, a natural cytotoxicity receptor (e.g., NCR1, NCR2, NCR3, NKp30, NKp44, NKp46, and/or CD158b), killer immunoglobulin-like receptor (KIR), and CD94 (also known as killer cell lectin-like receptor subfamily D, member 1 (KLRD1)) etc. In some embodiments, markers may be T cell markers (e.g., CD3, CD4, CD8, etc.).

### Methods of Use

A variety of diseases, disorders and/or conditions may be treated through use of technologies provided by the present disclosure. For example, in some embodiments, a disease, disorder and/or condition may be treated by introducing modified cells as described herein (e.g., edited iNK cells) to a subject. Examples of diseases that may be treated include, but not limited to, cancer, e.g., solid tumors, e.g., of the brain, prostate, breast, lung, colon, uterus, skin, liver, bone, pancreas, ovary, testes, bladder, kidney, head, neck, stomach, cervix, rectum, larynx, or esophagus; and hematological malignancies, e.g., acute and chronic leukemias, lymphomas, e.g., B-cell lymphomas including Hodgkin's and non-Hodgkin lymphomas, multiple myeloma and myelodysplastic syndromes.

In some embodiments, the present disclosure provides methods of treating a subject in need thereof by administering to the subject a composition comprising any of the cells described herein. In some embodiments, a therapeutic agent or composition may be administered before, during, or after the onset of a disease, disorder, or condition (including, e.g., an injury).

In particular embodiments, the subject has a disease, disorder, or condition, that can be treated by a cell therapy. In some embodiments, a subject in need of cell therapy is a subject with a disease, disorder and/or condition, whereby a cell therapy, e.g., a therapy in which a composition comprising a cell described herein, is administered to the subject, whereby the cell therapy treats at least one symptom associated with the disease, disorder, and/or condition. In some embodiments, a subject in need of cell therapy includes, but is not limited to, a candidate for bone marrow or stem cell transplant, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, and/or a subject who has or is at risk of having a viral infection or a disease associated with a viral infection.

### Pharmaceutical Compositions

In some embodiments, the present disclosure provides pharmaceutical compositions comprising one or more genetically modified cells described herein, e.g., an edited iNK cell described herein. In some embodiments, a pharmaceutical composition further comprises a pharmaceutically acceptable excipient. In some embodiments, a pharmaceutical composition comprises isolated pluripotent stem cell-derived hematopoietic lineage cells comprising at least 50%, 60%, 70%, 80%, 90%, 95%, 98%, or 99% T cells, NK cells, NKT cells, CD34+ HE cells or HSCs, e.g., genetically modified (e.g., edited) T cells, NK cells, NKT cells, CD34+ HE cells or HSCs. In some embodiments, a pharmaceutical composition comprises isolated pluripotent stem cell-derived hematopoietic lineage cells comprising about 95% to about 100% T cells, NK cells, NKT cells, CD34+ HE cells or HSCs, e.g., genetically modified (e.g., edited) T cells, NK cells, NKT cells, CD34+ HE cells or HSCs.

In some embodiments, a pharmaceutical composition of the present disclosure comprises an isolated population of pluripotent stem cell-derived hematopoietic lineage cells, wherein the isolated population has less than about 0.1%, 0.5%, 1%, 2%, 5%, 10%, 15%, 20%, 25%, or 30% T cells, NK cells, NKT cells, CD34+ HE cells or HSCs, e.g., genetically modified (e.g., edited) T cells, NK cells, NKT cells, CD34+ HE cells or HSCs. In some embodiments, an isolated population of pluripotent stem cell-derived hematopoietic lineage cells has more than about 0.1%, 0.5%, 1%, 2%, 5%, 10%, 15%, 20%, 25%, or 30% T cells, NK cells, NKT cells, CD34+ HE cells or HSCs, e.g., genetically modified (e.g., edited) T cells, NK cells, NKT cells, CD34+ HE cells or HSCs. In some embodiments, an isolated population of pluripotent stem cell-derived hematopoietic lineage cells has about 0.1% to about 1%, about 1% to about 3%, about 3% to about 5%, about 10%-about 15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% T cells, NK cells, NKT cells, CD34+ HE cells or HSCs, e.g., genetically modified (e.g., edited) T cells, NK cells, NKT cells, CD34+ HE cells or HSCs.

In some embodiments, an isolated population of pluripotent stem cell-derived hematopoietic lineage cells comprises about 0.1%, about 1%, about 3%, about 5%, about 10%, about 15%, about 20%, about 25%, about 30%, about 35%, about 40%, about 45%, about 50%, about 60%, about 70%, about 80%, about 90%, about 95%, about 98%, about 99%, or about 100% T cells, NK cells, NKT cells, CD34+ HE cells or HSCs, e.g., genetically modified (e.g., edited) T cells, NK cells, NKT cells, CD34+ HE cells or HSCs.

As one of ordinary skill in the art would understand, both autologous and allogeneic cells can be used in adoptive cell therapies. Autologous cell therapies generally have reduced infection, low probability for GVHD, and rapid immune reconstitution relative to other cell therapies. Allogeneic cell therapies generally have an immune mediated graft-versus-malignancy (GVM) effect, and low rate of relapse relative to other cell therapies. Based on the specific condition(s) of the subject in need of the cell therapy, one of ordinary skill in the art would be able to determine which specific type of therapy(ies) to administer.

In some embodiments, a pharmaceutical composition comprises pluripotent stem cell-derived hematopoietic lineage cells that are allogeneic to a subject. In some embodiments, a pharmaceutical composition comprises pluripotent stem cell-derived hematopoietic lineage cells that are autologous to a subject. For allogeneic transplantation, the isolated population of pluripotent stem cell-derived hematopoietic lineage cells can be cither a complete or partial HLA-match with patient subject. In some embodiments, the pluripotent stem cell-derived hematopoietic lineage cells are not HLA-matched to a subject.

In some embodiments, pluripotent stem cell-derived hematopoietic lineage cells can be administered to a subject without being expanded ex vivo or in vitro prior to administration. In particular embodiments, an isolated population of derived hematopoietic lineage cells is modulated and treated ex vivo using one or more agents to obtain immune cells with improved therapeutic potential. In some embodiments, the modulated population of derived hematopoietic lineage cells can be washed to remove the treatment agent(s), and the improved population can be administered to a subject without further expansion of the population in vitro. In some embodiments, an isolated population of derived hematopoietic lineage cells is expanded prior to modulating the isolated population with one or more agents.

In some embodiments, an isolated population of derived hematopoietic lineage cells can be genetically modified (e.g., by recombinant methods) to express TCR, CAR or other proteins. For genetically engineered derived hematopoietic lineage cells that express recombinant TCR or CAR, whether prior to or after genetic modification of the cells, the cells can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

### Cancers

Any cancer can be treated using a cell or pharmaceutical composition described herein. Exemplary therapeutic targets of the present disclosure include cancer cells from the bladder, blood, bone, bone marrow, brain, breast, colon, esophagus, eye, gastrointestinal system, gum, head, kidney, liver, lung, nasopharynx, neck, ovary, prostate, skin, stomach, testis, tongue, or uterus. In addition, a cancer may specifically be of the following non-limiting histological type: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; B-cell lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is a breast cancer. In some embodiments, the cancer is colorectal cancer (e.g., colon cancer). In some embodiments, the cancer is gastric cancer. In some embodiments, the cancer is RCC. In some embodiments, the cancer is non-small cell lung cancer (NSCLC). In some embodiments, the cancer is head and neck cancer.

In some embodiments, solid cancer indications that can be treated with iNK cells (e.g., genetically modified iNK cells, e.g., edited iNK cells) provided herein, either alone or in combination with one or more additional cancer treatment modality, include: bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, and soft tissue sarcomas. In some embodiments, hematological cancer indications that can be treated with the iNK cells (e.g., genetically modified iNK cells, e.g., edited iNK cells) provided herein, either alone or in combination with one or more additional cancer treatment modalities, include: ALL, CLL, NHL, DLBCL, AML, CML, and multiple myeloma (MM).

Examples of cellular proliferative and/or differentiative disorders of the lung include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders involving the colon include, but are not limited to, tumors of the colon, such as non-neoplastic polyps, adenomas, familial syndromes, colorectal carcinogenesis, colorectal carcinoma, and carcinoid tumors.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Exemplary useful additional cancer treatment modalities include, but are not limited to: chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylornithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

In some embodiments, cells described herein (e.g., cells modified using methods of the disclosure) are used in combination with one or more cancer treatment modalities that facilitate the induction of antibody dependent cellular cytotoxicity (ADCC) (see e.g., Janeway's Immunobiology by K. Murphy and C. weaver). In some embodiments, such a cancer treatment modality is an antibody, e.g., an antibody described herein. In some embodiments, cells described herein (e.g., cells modified using methods of the disclosure) are used in combination with one or more cancer treatment modalities that facilitate the induction of antibody dependent cellular cytotoxicity (ADCC), wherein the cancer treatment modality is an antibody or appropriate fragment thereof targeting CD20, TNFα, HER2, CD52, IgE, EGFR, VEGF-A, ITGA4, CTLA-4, CD30, VEGFR2, α4β7 integrin, CD19, CD3, PD-1, GD2, CD38, SLAMF7, PDGFRα, PD-L1, CD22, CD33, IFNγ, CD79β, or any combination thereof.

In some embodiments, such an antibody is Trastuzumab. In some embodiments, such an antibody is Rituximab. In some embodiments, such an antibody is Rituximab, Palivizumab, Infliximab, Trastuzumab, Alemtuzumab, Adalimumab, Ibritumomab tiuxetan, Omalizumab, Cetuximab, Bevacizumab, Natalizumab, Panitumumab, Ranibizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Obinutuzumab, Siltuximab, Ramucirumab, Vedolizumab, Blinatumomab, Nivolumab, Pembrolizumab, Idarucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Inotuzumab ozogamicin, Brodalumab, Guselkumab, Dupilumab, Sarilumab, Avelumab, Ocrelizumab, Emicizumab, Benralizumab, Gemtuzumab ozogamicin, Durvalumab, Burosumab, Lanadelumab, Mogamulizumab, Erenumab, Galcanezumab, Tildrakizumab, Cemiplimab, Emapalumab, Fremanezumab, Ibalizumab, Moxetumomab pasudodox, Ravulizumab, Romosozumab, Risankizumab, Polatuzumab vedotin, Brolucizumab, or any combination thereof (see e.g., Lu et al., Development of therapeutic antibodies for the treatment of diseases. Journal of Biomedical Science, 2020).

In some embodiments, cells described herein are utilized in combination with checkpoint inhibitors. Examples of suitable combination therapy checkpoint inhibitors include, but are not limited to, antagonists of PD-1 (Pdcdl, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A2aR, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, inhibitory KIR (for example, 2DL1, 2DL2, 2DL3, 3DL1, and 3DL2), or any suitable combination thereof.

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavychain-only antibody (VNAR), Ig NAR, chimeric antibodies, recombinant antibodies, or antibody fragments thereof. Non-limiting examples of antibody fragments include Fab, Fab′, F(ab)′2, F(ab)′3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirimumab (anti-KIR), monalizumab (anti-NKG2A), nivolumab (anti-PD1 mAb), pembrolizumab (anti-PD 1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2): 103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, miR-29c, and/or any suitable combination thereof.

In some embodiments, cells described herein (e.g., cells modified using methods of the disclosure) are used in combination with one or more cancer treatment modalities such as exogenous interleukin (IL) dosing. In some embodiments, an exogenous IL provided to a patient is IL-15. In some embodiments, systemic IL-15 dosing when used in combination with cells described herein is reduced when compared to standard dosing concentrations (see e.g., Waldmann et al., IL-15 in the Combination Immunotherapy of Cancer. Front. Immunology, 2020).

Other compounds that are effective in treating cancer are known in the art and described herein that are suitable for use with the compositions and methods of the present disclosure as additional cancer treatment modalities are described, for example, in the “Physicians' Desk Reference, 62nd edition. Oradell, N.J.: Medical Economics Co., 2008”. Goodman & Gilman's “The Pharmacological Basis of Therapeutics, Eleventh Edition. McGraw-Hill, 2005”, “Remington: The Science and Practice of Pharmacy, 20th Edition. Baltimore, Md.: Lippincott Williams & Wilkins, 2000.”, and “The Merck Index, Fourteenth Edition. Whitehouse Station, N.J.: Merck Research Laboratories, 2006”, incorporated herein by reference in relevant parts.

All publications, patents and patent applications cited herein, whether supra or infra, are hereby incorporated by reference in their entirety.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of is meant including, and limited to, whatever follows the phrase “consisting of:” Thus, the phrase “consisting of indicates that the listed elements are required or mandatory, and that no other elements may be present. By “consisting essentially of is meant including any elements listed after the phrase, and limited to other elements that do not interfere with or contribute to the activity or action specified in the disclosure for the listed elements. Thus, the phrase “consisting essentially of indicates that the listed elements are required or mandatory, but that no other elements are optional and may or may not be present depending upon whether or not they affect the activity or action of the listed elements.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. The contents of database entries, e.g., NCBI nucleotide or protein database entries provided herein, are incorporated herein in their entirety. Where database entries are subject to change over time, the contents as of the filing date of the present application are incorporated herein by reference. Aspects of the embodiments can be modified, if necessary to employ concepts of the various patents, applications and publications to provide yet further embodiments.

The disclosure is further illustrated by the following examples. The examples are provided for illustrative purposes only. They are not to be construed as limiting the scope or content of the disclosure in any way.

### EXAMPLES

### Example 1: Generating Edited iPSC Cells Using Cas12a and Testing Effect of Activin a on Pluripotency

To generate natural killer cells from pluripotent stem cells, a representative induced pluripotent stem cell (iPSC) line was generated and designated “PCS-201”. This line was generated by reprogramming adult male human primary dermal fibroblasts purchased from ATCC (ATCC® PCS-201-012) using a commercially available non-modified RNA reprogramming kit (Stemgent/Reprocell, USA). The reprogramming kit contains non-modified reprogramming mRNAs (OCT4, SOX2, KLF4, cMYC, NANOG, and LIN28) with immune evasion mRNAs (E3, K3, and B18R) and double-stranded microRNAs (miRNAs) from the 302/367 clusters. Fibroblasts were seeded in fibroblast expansion medium (DMEM/F12 with 10% FBS). The next day, media was switched to Nutristem medium and daily overnight transfections were performed for 4 days (day 1 to 4). Primary iPSC colonies appeared on day 7 and were picked on day 10-14. Picked colonies were expanded clonally to achieve a sufficient number of cells to establish a master cell bank. The parental line chosen from this process and used for the subsequent experiments passed standard quality controls, including confirmation of stemness marker expression, normal karyotype and pluripotency.

To generate edited iPSC cells, the PCS-201 (PCS) cells were electroporated with a Cas 12a RNP designed to cut at the target gene of interest. Briefly, the cells were treated 24 hours prior to transfection with a ROCK inhibitor (Y27632). On the day of transfection, a single cell solution was generated using accutase and 500,000 PCS iPS cells were resuspended in the appropriate electroporation buffer and Cas12a RNP at a final concentration of 2 μM. When two RNPs were added simultaneously, the total RNP concentration was 4 μM (2+2). This solution was electroporated using a Lonza 4D electroporator system. Following electroporation, the cells were plated in 6-well plates in mTESR media containing CloneR (Stemcell Technologies). The cells were allowed to grow for 3-5 days with daily media changes, and the CloneR was removed from the media by 48 hours post electroporation. To pick single colonies, the expanded cells were plated at a low density in 10 cm plates after resuspending them in a single cell suspension. Rock inhibitor was used to support the cells during single cell plating for 3-5 days post plating depending on the size of the colonies on the plate. After 7-10 days, sufficiently sized colonies with acceptable morphology were picked and plated into 24-well plates. The picked colonies were expanded to sufficient numbers to allow harvesting of genomic DNA for subsequent analysis and for cell line cryopreservation. Editing was confirmed by NGS and selected clones were expanded further and banked. Ultimately, karyotyping, stemness flow, and differentiation assays were performed on a subset of selected clones.

Two target genes of interest were CISH and TGFβRII, both of which were hypothesized to enhance natural killer cell function. As the TGFβ:TGFβRII pathway is believed to be involved in the maintenance of pluripotency, it was hypothesized that a functional deletion of TGFβRII in iPSCs could lead to differentiation and prevent generation of TGFβRII edited iPSCs. Due to the convergence of Activin receptor signaling and TGFβRII signaling in regulating SMAD2/3 and other intracellular molecules, it was hypothesized that Activin A could replace TGFβ in commercially available pluripotent stem cell medias to generate edited lines. To test this hypothesis, the pluripotency of unedited and TGFβRII edited iPSCs grown with Activin A was assessed. Several different culture medias were utilized: “E6” (Essential 6™ Medium, #A1516401, ThermoFisher), which lacks TGFβ, “E7”, which was E6 supplemented with 100 ng/ml of bFGF (Peprotech, #100-18B), “E8” (Essential 8™ Medium, #A1517001, ThermoFisher), and “E7+ActA”, which was E6 supplemented with 100 ng/ml of bFGF and varying concentrations of Activin A (Peprotech #120-14P). Typically, E6 and E7 medias are typically insufficient to maintain the stemness and pluripotency of PSCs over multiple passages in culture.

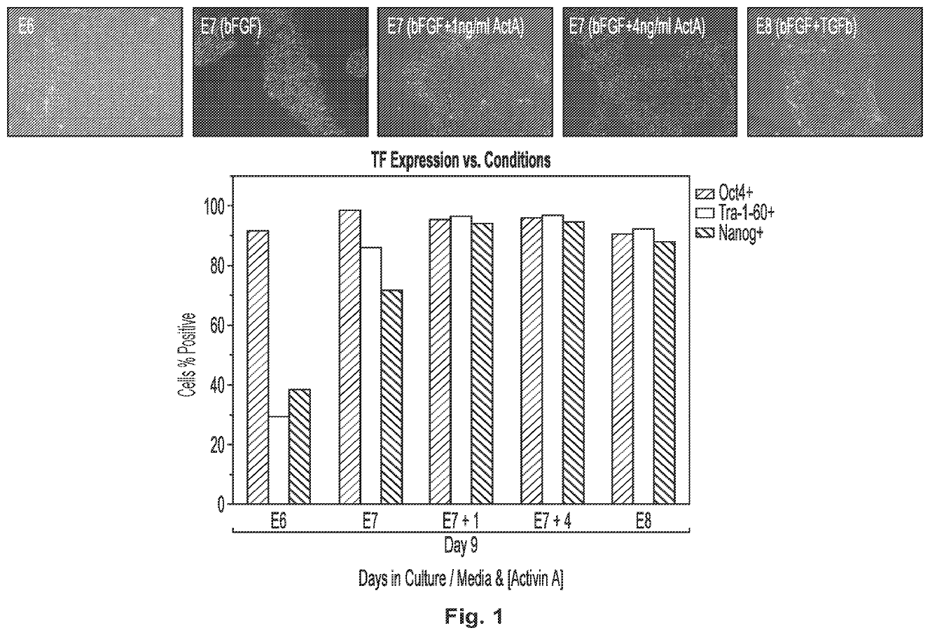

In order to determine whether Activin A could maintain PCS iPSCs in the absence of exogenous TGFβ, unedited PCS iPSCs were plated on a LaminStem™ 521 (Biological Industries) coated 6-well plate and cultured in E6, E7, E8 or E7+ActA (with Activin A at two different concentrations-1 ng/ml and 4 ng/ml). After 2 passages, the cells were assessed for morphology and stemness marker expression. Morphology was assessed using a standard phase contrast setting on an inverted microscope. Colonies with defined edges and non-differentiated cells typical of iPSC colonies, were deemed to be stem like. To confirm the morphological observations, the expression of standard iPS cell stemness markers was measured using intracellular flow cytometry. Briefly, cells were dissociated, stained for extracellular markers, and then fixed overnight and permeabilized using the reagents and standard protocol from the Foxp3/Transcription Factor Staining Buffer Set (eBioscience™). Cells were stained for flow cytometric analysis with anti-human TRA-1-60-R_AF®488 (Biolegend®; Clone TRA-1-60-R), anti-Human Nanog_AF®647 (BD Pharmingen™; Clone N31-355), and anti-Oct4 (Oct3)_PE (Biolegend®; Clone 3A2A20). Cells were recorded on a NovoCyte Quanteon Flow Cytometer (Agilent) and analyzed using FlowJo (FlowJo, LLC). As shown in FIG. 1, both 1 ng/mL and 4 ng/ml of Activin A was sufficient to maintain pluripotency with equivalent stemness marker expression to the cells grown in E8. As expected, cells grown in E6 and E7 (which lacked TGFβ) did not maintain stemness gene expression to the same degree as E8, indicating the loss of iPSC stemness in the absence of TGFβ or Activin A. These results suggest that Activin A can supplement iPSC stemness in the absence of TGFβ signaling.

Given the demonstration that Activin A could support iPSC stemness in the absence of TGFβ. TGFβRII knockout (“KO”) iPSCs, CISH KO iPSCs, and TGFβRII/CISH double knockout (“DKO”) iPSC lines were generated. Specifically, iPSCs were edited using an RNP having an engineered Cas12a with three amino acid substitutions (M537R, F870L, and H800A (SEQ ID NO: 1148)) and a gRNA specific for CISH or TGFβRII. To make CISH/TGFβRII DKO iPSCs, iPSCs were treated with an RNP targeting CISH and an RNP targeting TGFβRII simultaneously. The particular guide RNA sequences of Table 10 were used for editing of CISH and TGFβRII. Both guides were generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence.

TABLE 10
Guide RNA sequences

| | gRNA | |
|---|---|---|
| | Targeting | Full Length |
| | Domain | gRNA |
| Target | Sequence | Sequence |
| CISH 7050 | GGUGUACAGC | ATGTGTTTTTGTCAA |
| | AGUGGCUGGU | AAGACCTTTTrUrAr |
| | (SEQ ID | ArUrUrUrCrUrArC |
| | NO: 1155) | rUrCrUrUrGrUrAr |
| | | GrArUrGrGrUrGrU |
| | | rArCrArGrCrArGr |
| | | UrGrGrCrUrGrGrU |
| | | (SEQ ID |
| | | NO: 1156) |
| TGFβRII | UGAUGUGAG | ATGTGTTTTTGTCAA |
| 24026 | AUUUUCCAC | AAGACCTTTTrUrAr |
| | CU | ArUrUrUrCrUrArC |
| | (SEQ ID | rUrCrUrUrGrUrAr |
| | NO: 1157) | GrArUrUrGrArUrG |
| | | rUrGrArGrArUrUr |
| | | UrUrCrCrArCrCrU |
| | | (SEQ ID |
| | | NO: 1158) |

The edited clones were generated as described above with a minor modification for the cells treated with TGFβRII RNPs. Briefly, TGFβRII-edited PCS iPSCs and TGFβRII/CISH edited PCS iPSCs were plated after electroporation at the 6-well stage in the mTESR supplemented with 10 ng/ml of Activin A in order to support the generation of edited clones. The cells were cultured with 10 ng/ml of Activin A through the cell colony picking and early expansion stages. Colonies assessed as having the correct single KO (CISH KO or TGFβRII KO) or double KO (CISH/TGFβRII DKO) were picked and expanded (clonal selection).

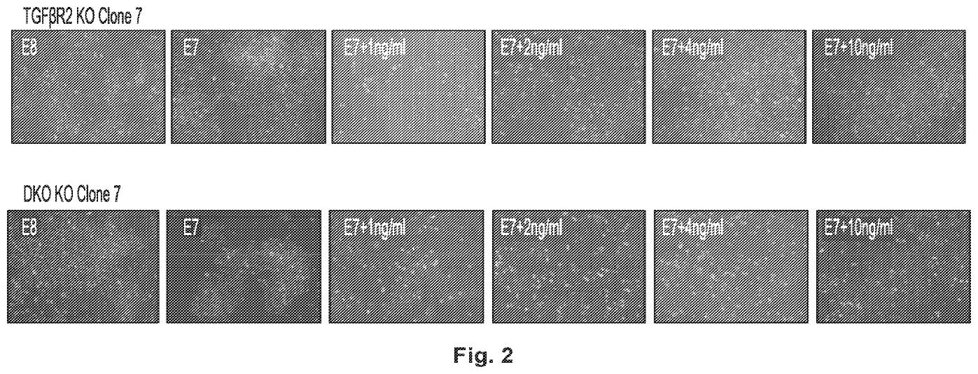

To determine the optimal concentration of Activin A for culturing of TGFβRII KO and TGFβRII/CISH DKO iPSCs, a slightly expanded concentration curve was tested as shown FIG. 2. Similar to the assessment performed previously, the iPSCs were cultured in a Matrigel-treated 6-well plate with concentrations of 1 ng/ml, 2 ng/ml, 4 ng/ml and 10 ng/ml Activin A. As shown in FIG. 2, TGFβRII KO or CISH/TGFβRII DKO cells cultured in E7 medium supplemented with 4 ng/mL Activin A for 19 days (over 5 passages) maintained a wild type morphology. FIG. 3 shows the morphology of TGFβRII KO PCS-201 hiPSC Clone 9.

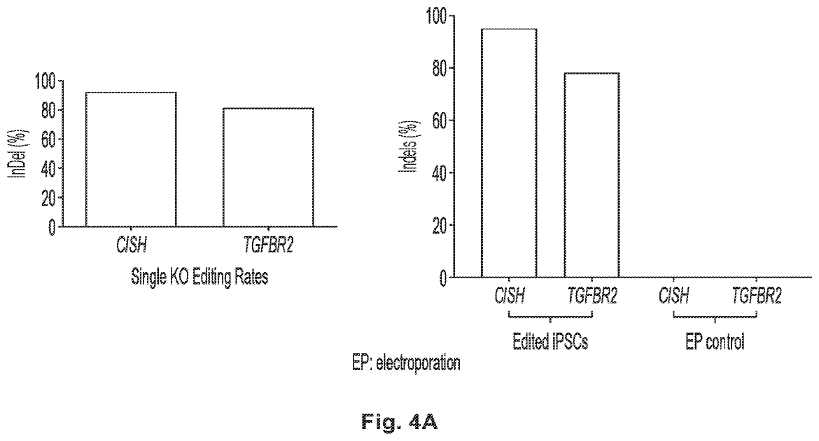

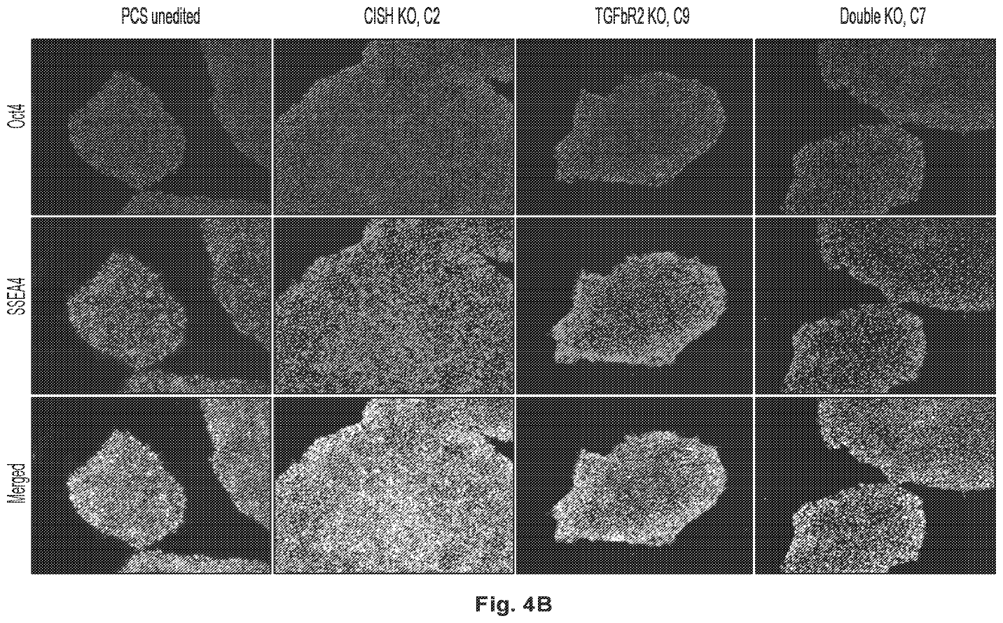

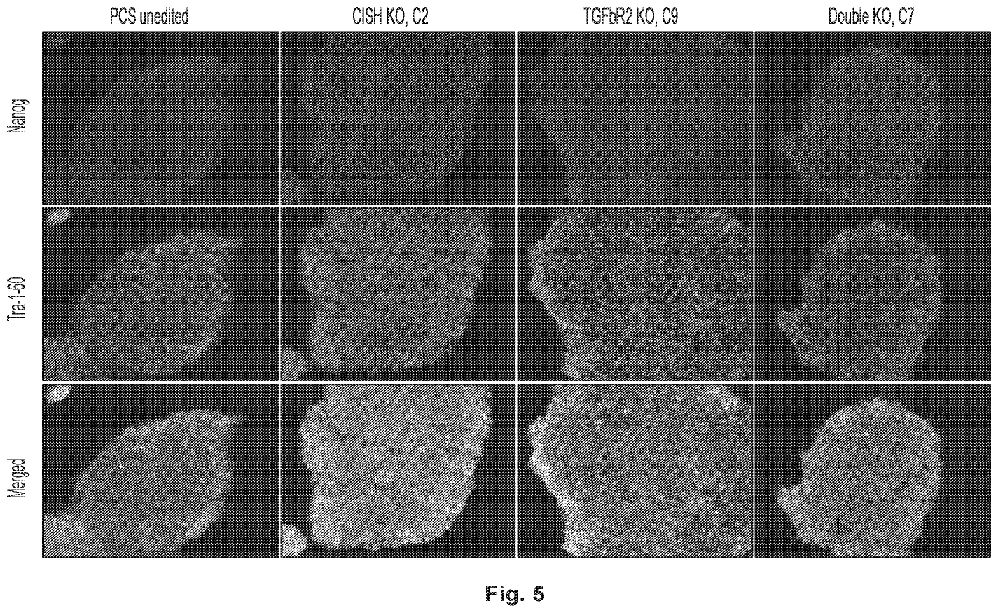

As shown in FIG. 4A, the initial editing efficiency of the iPSCs treated simultaneously with the CISH and TGFβRII RNPs (prior to clonal selection) was high, with 95% of the CISH alleles edited and 78% of the TGFβRII alleles edited. Unedited iPSC controls did not have indels at either loci. iPSCs that were treated with CISH or TGFβRII RNPs individually showed 93% and 82% editing rates prior to clone selection (depicted in FIG. 4A). The KO cell lines (CISH KO iPSCs, TGFβRII KO iPSCs, and CISH/TGFβRII DKO iPSCs) were subsequently assessed for the presence of pluripotency markers Oct4, SSEA4, Nanog, and Tra-1-60 after culturing in the presence of supplemental Activin A. As shown in FIGS. 4B and 5, culturing the KO cell lines in Activin A maintained expression of these pluripotency markers.

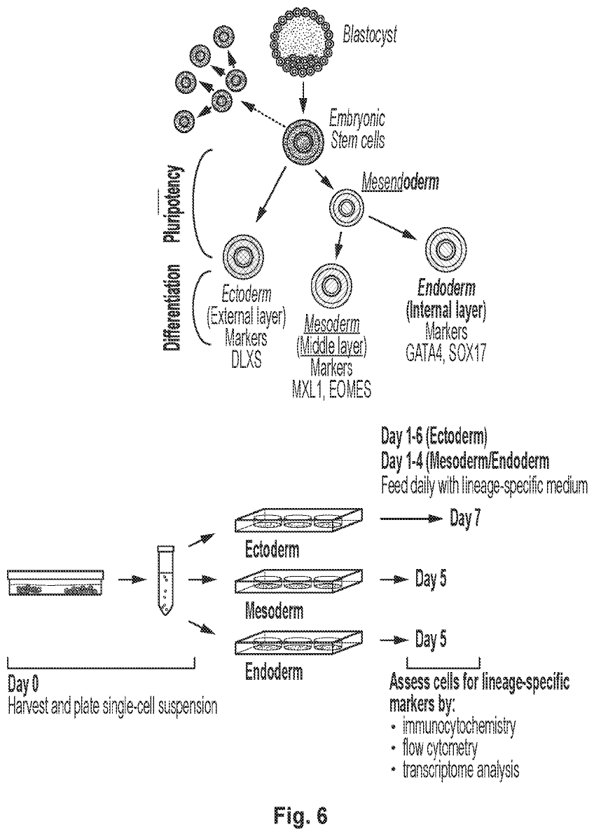

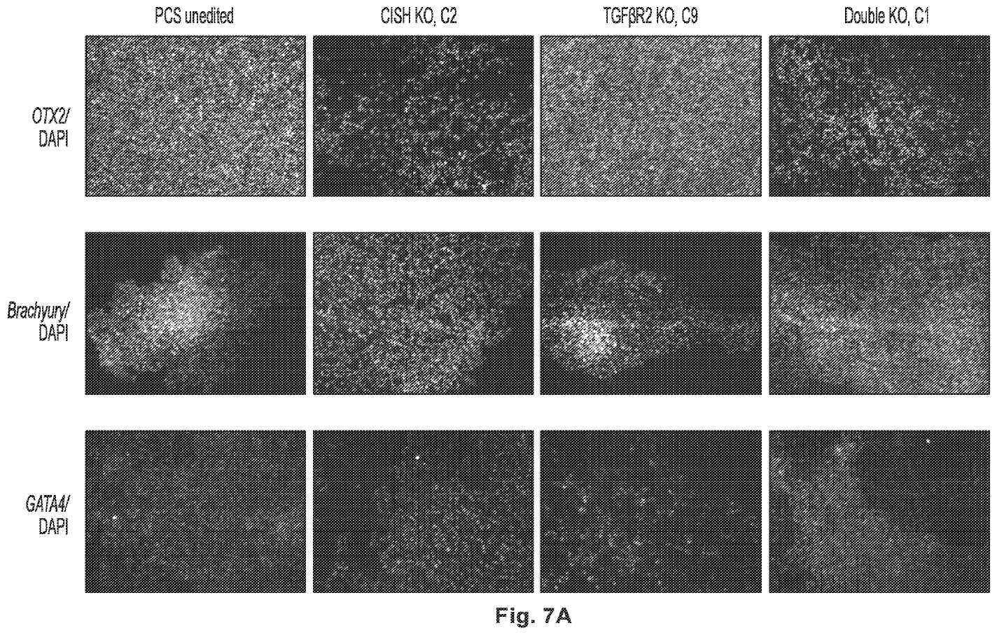

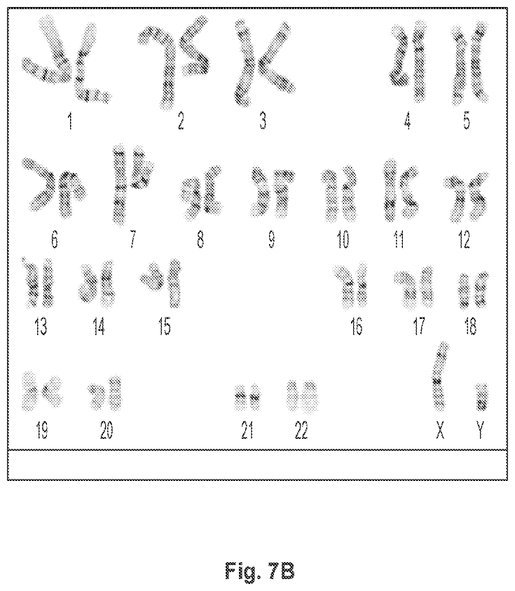

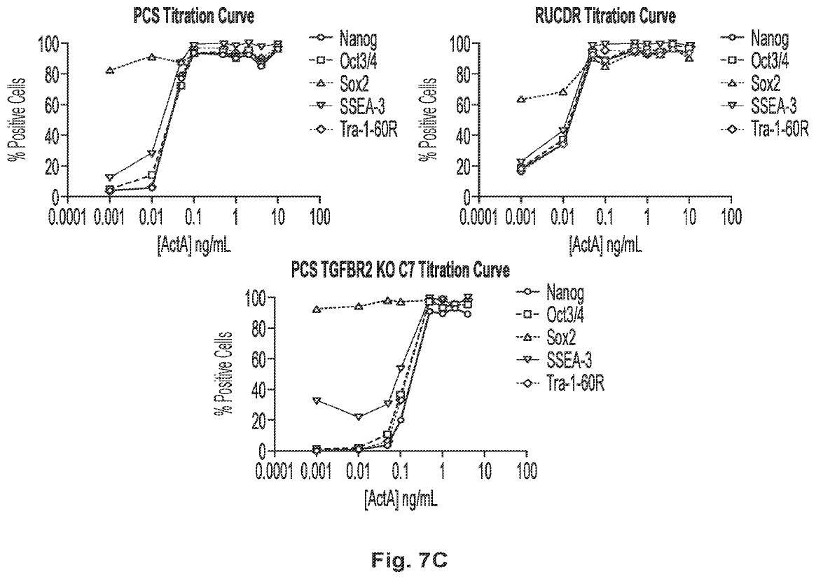

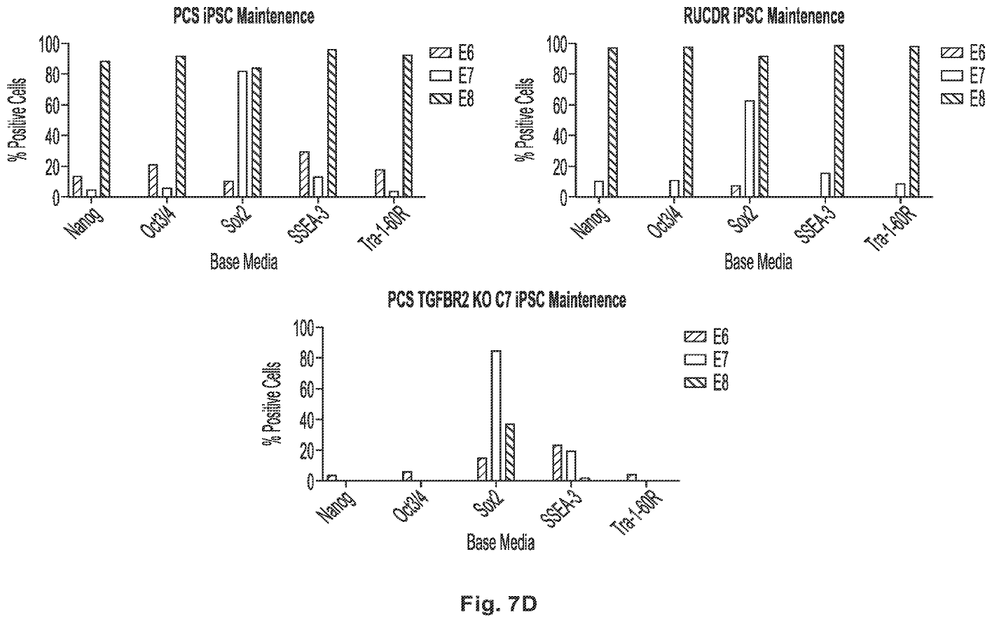

The KO iPSC lines cultured in Activin A were next assessed for their capacity to differentiate using the STEMdiff™ Trilineage Differentiation Kit assay (from STEMCELL Technologies Inc., Vancouver, BC, CA) as depicted schematically in FIG. 6. As shown in FIG. 7A, culturing the single KO (TGFβRII KO iPSCs or CISH KO iPSCs) and DKO (TGFβRII/CISH DKO iPSCs) cell lines in media with supplemental Activin A maintained their ability to differentiate into early progenitors of all 3 germ layers, as shown by expression of ectoderm (OTX2), mesoderm (brachyury), and endoderm (GATA4) markers (FIG. 7A). The unedited PCS control cells were also able to express each of these markers.

The edited iPSCs were next karyotyped to determine whether the Cas12a editing caused large genetic abnormalities, such as translocations. As shown in FIG. 7B, the cells had normal karyotypes with no translocation between the cut sites.

To further support the results described above, an expanded Activin A concentration curve was performed on the unedited parental PSC line, an edited TGFβRII KO iPSC clone (C7), and an additional representative (unedited) cell line designated RUCDR (RUCDR Infinite Biologics group, Piscaway NJ). At the outset, the iPSCs were seeded at 1e5 cells per well in a 1× LaminStem™ 521 (Biological Industries) coated 12-well plate. Cells were then passaged 10 times over ˜40-50 days using 0.5 mM EDTA in 1×PBS dissociation and Y-27632 (Biological Industries) until wells achieved >75% confluency. Cells were cultured in Essential 6™ Medium (Gibco), TeSR™-E7™, and TeSR™-E8™ (StemCell Technologies) for controls and titrated using TeSR™-E7™ supplemented with E. coli-derived recombinant human/murine/rat Activin A (PeproTech) spanning a 4-log concentration dosage (0.001-10 ng/mL). Following 5 and 10 passages, cells were dissociated and then fixed overnight and permeabilized using the reagents and standard protocol from the Foxp3/Transcription Factor Staining Buffer Set (eBioscience™). Cells were stained for flow cytometric analysis with anti-human TRA-1-60-R_AF®488 (Biolegend®; Clone TRA-1-60-R), anti Sox2_PerCP-Cy™5.5 (BD Pharmingen™; Clone O30-678), anti-Human Nanog_AF®647 (BD Pharmingen™; Clone N31-355), anti-Oct4 (Oct3)_PE (Biolegend®; Clone 3A2A20), and anti-human SSEA-4_PE/Dazzle™ 594 (Biolegend®; Clone MC-813-70). Cells were recorded on a NovoCyte Quanteon Flow Cytometer (Agilent) and analyzed using FlowJo (FlowJo, LLC). FIG. 7C shows the titration curves for the tested iPSC lines. The minimum concentration of Activin A required to maintain each line varied slightly, with the TGFβRII KO iPSCs requiring a higher baseline amount of Activin A as compared to the parental control (0.5 ng/ml vs 0.1 ng/ml). In all 3 cell lines, 4 ng/ml was well above the minimum amount of Activin A necessary to maintain stemness marker expression over an extended culture period. FIG. 7D shows the stemness marker expression in the cells culture with the base medias alone (no Activin A). As expected, the TGFβRII KO iPSCs did not maintain expression, while the two unedited lines were able to maintain stemness marker expression in E8.

### Example 2: Differentiation of Edited CISH KO, TGFβRII KO, and CISH/TGFβRII DKO iPSCs into iNK Cells Exhibiting Enhanced Function

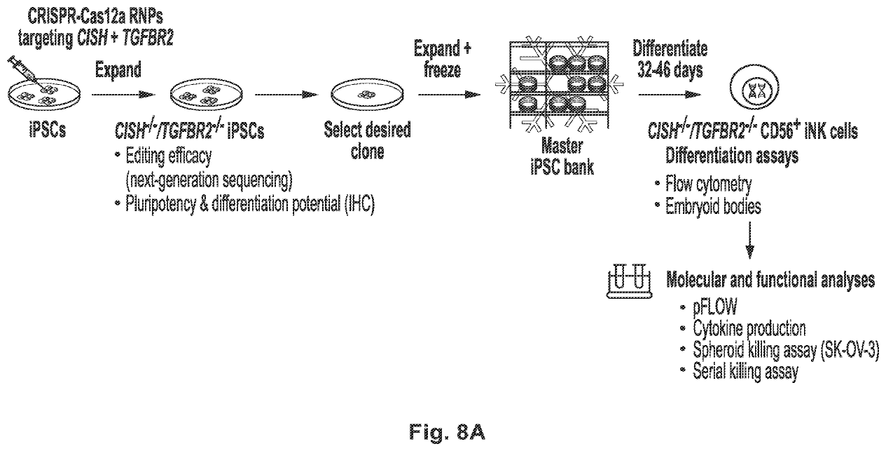

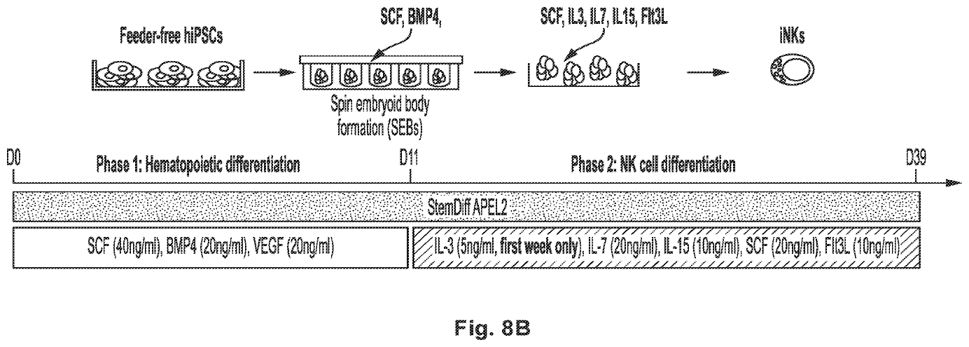

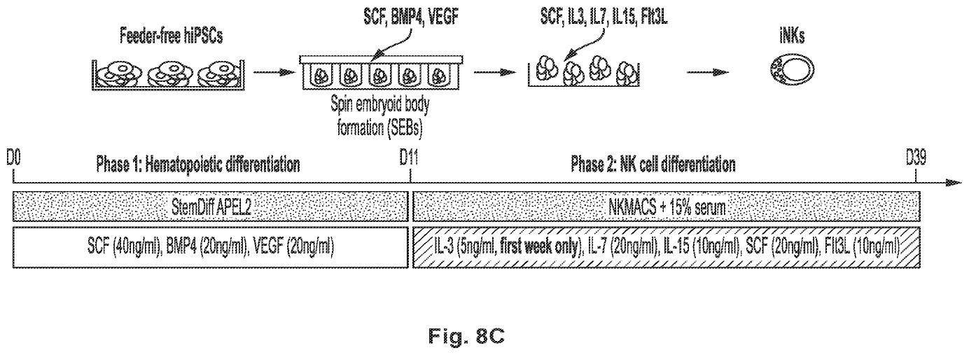

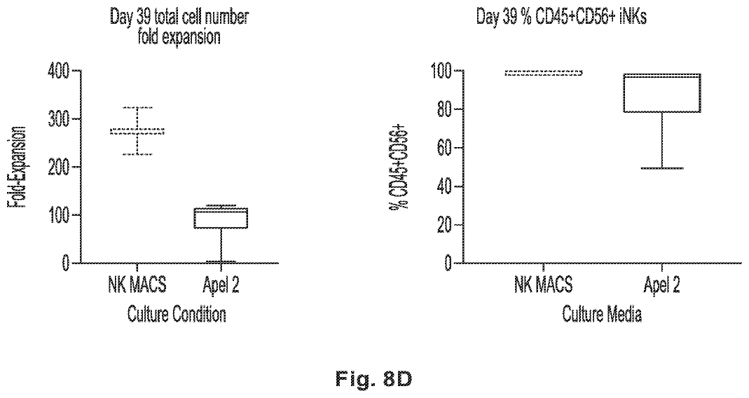

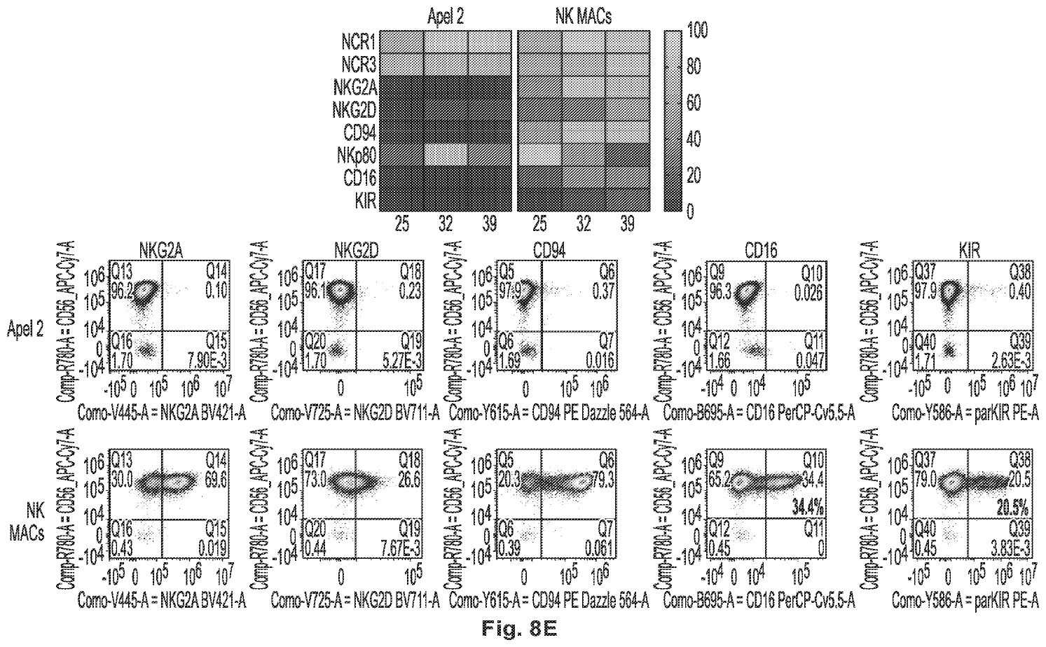

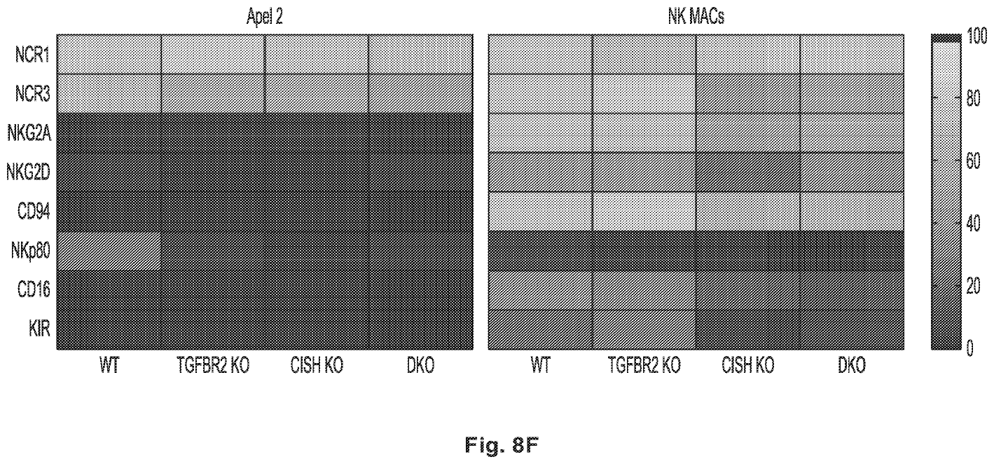

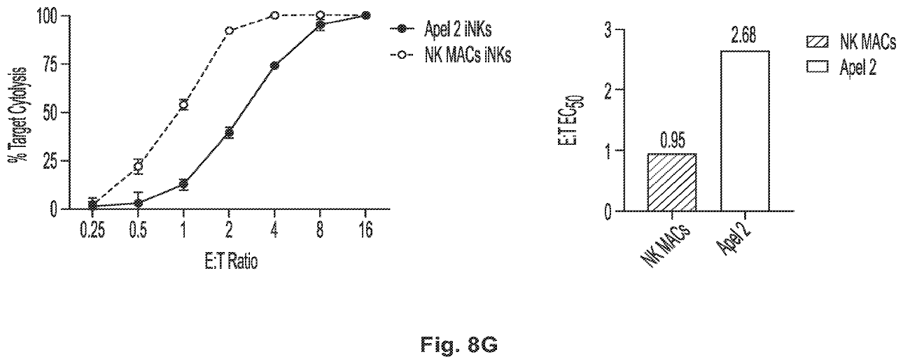

FIG. 8A depicts a schematic of an exemplary workflow for development of a CRISPR-Cas12a-edited iPSC platform for generation of enhanced CD56+ iNK cells. As shown in FIG. 8A, the CISH and TGFβRII genes are targeted in iPSCs via delivery of RNPs to the cells using electroporation to generate CISH/TGFβRII DKO iPSCs. iPSCs with the desired edits at both the CISH and TGFβRII genes can then be selected and expanded to create a master iPSC bank. Edited cells from the iPSC master bank can then be differentiated into CD56+CISH/TGFβRII DKO INK cells.

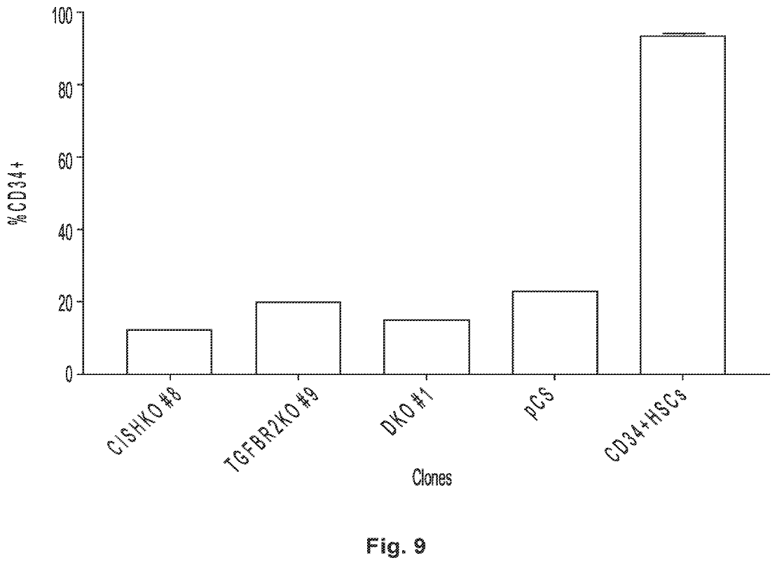

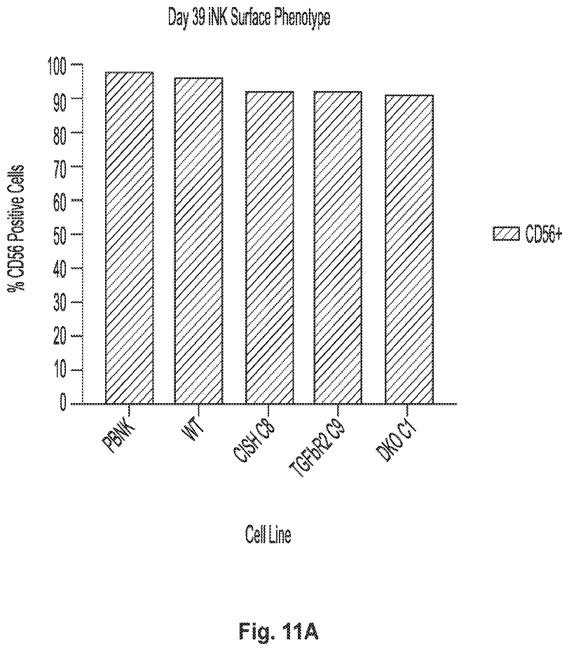

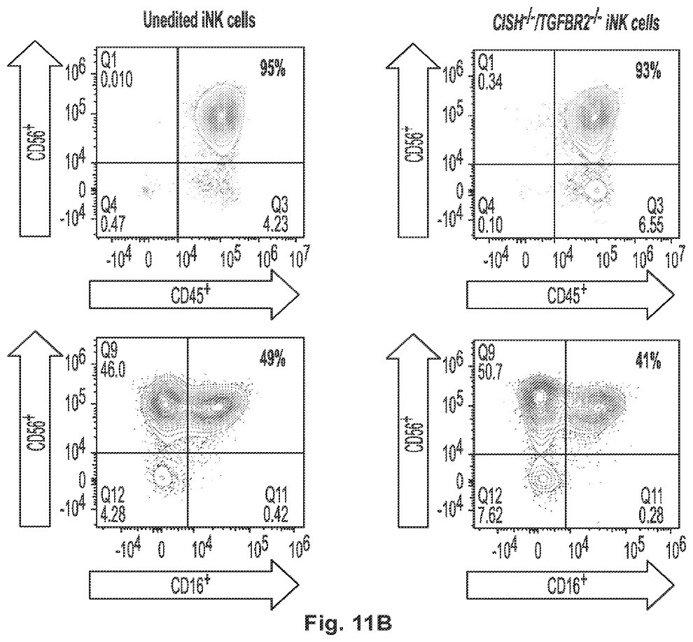

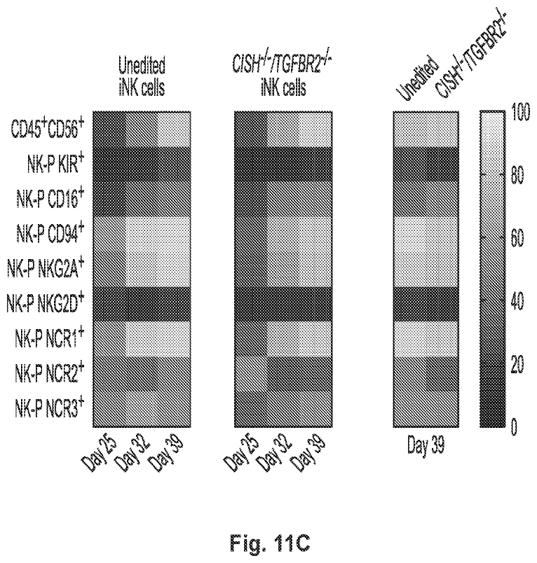

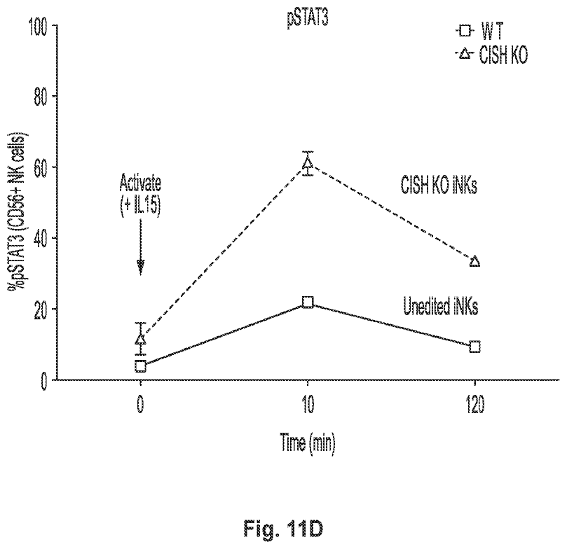

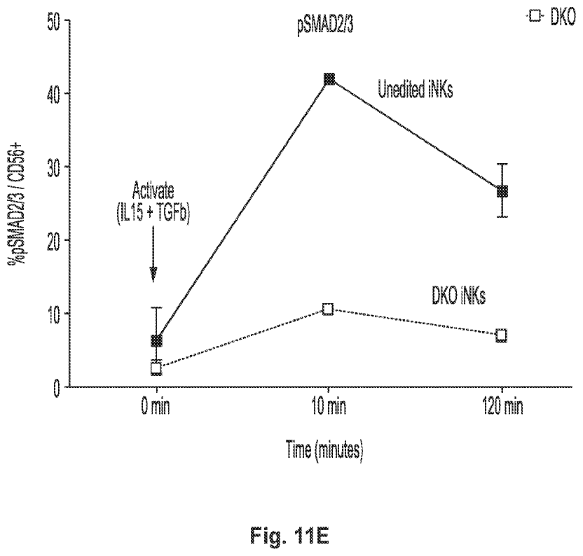

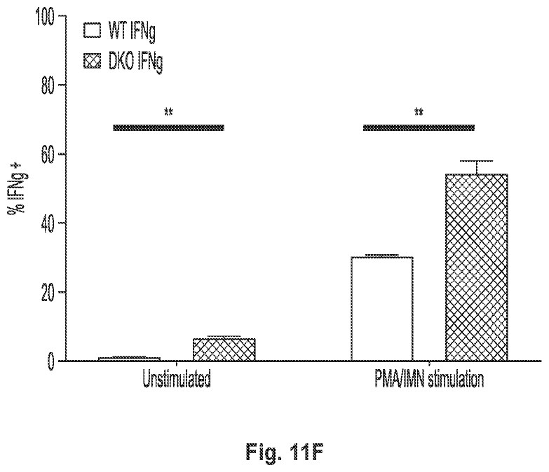

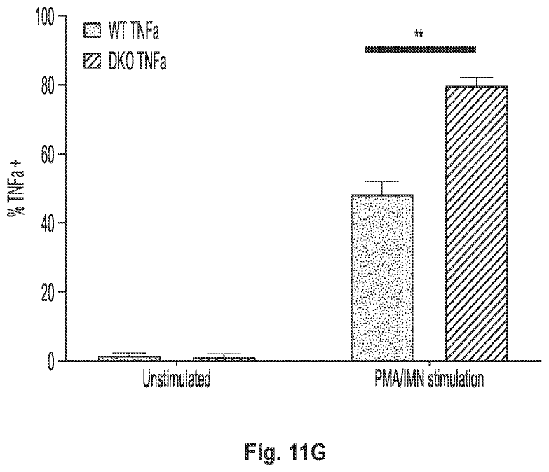

FIGS. 8B and 8C depict two exemplary schematics of the process of differentiating iPSCs into iNK cells. As shown in FIGS. 8B and 8C, edited cells (or unedited control cells) were differentiated using a two-phase process. First, in the “hematopoietic differentiation phase,” hiPSCs (edited and unedited) were cultured in StemDiff™ APEL2™ medium (StemCell Technologies) with SCF (40 ng/mL), BMP4 (20 ng/mL), and VEGF (20 ng/mL) from days 0-10, to produce spin embryoid bodies (SEBs). As shown in FIG. 8B, SEBs were then cultured from days 11-39 in StemDiff™ APEL2™ medium comprising IL-3 (5 ng/mL, only present for the first week of culture), IL-7 (20 ng/ml), IL-15 (10 ng/ml), SCF (20 ng/mL), and Flt3L (10 ng/mL) in an NK cell differentiation phase. CISH KO iPSCs, TGFβRII KO iPSCs, CISH/TGFβRII DKO iPSCs, and unedited wild-type iPSC lines (PCS), were differentiated into iNKs according to the schematic in FIG. 8B, and then characterized to assess whether they exhibited a phenotype congruent with NK cells (see FIGS. 9, 10, and 11A). CISH KO iPSCs, TGFβRII KO iPSCs, CISH/TGFβRII DKO iPSCs, and unedited wild-type iPSC lines, described in FIGS. 11B, 11C, 12B, 12C, and 13 were also differentiated into iNKs utilizing the alternative method shown in FIG. 8C, and then characterized to assess whether they exhibited a phenotype congruent with NK cells (see FIGS. 11B, 11C, 12B, 12C, and 13).

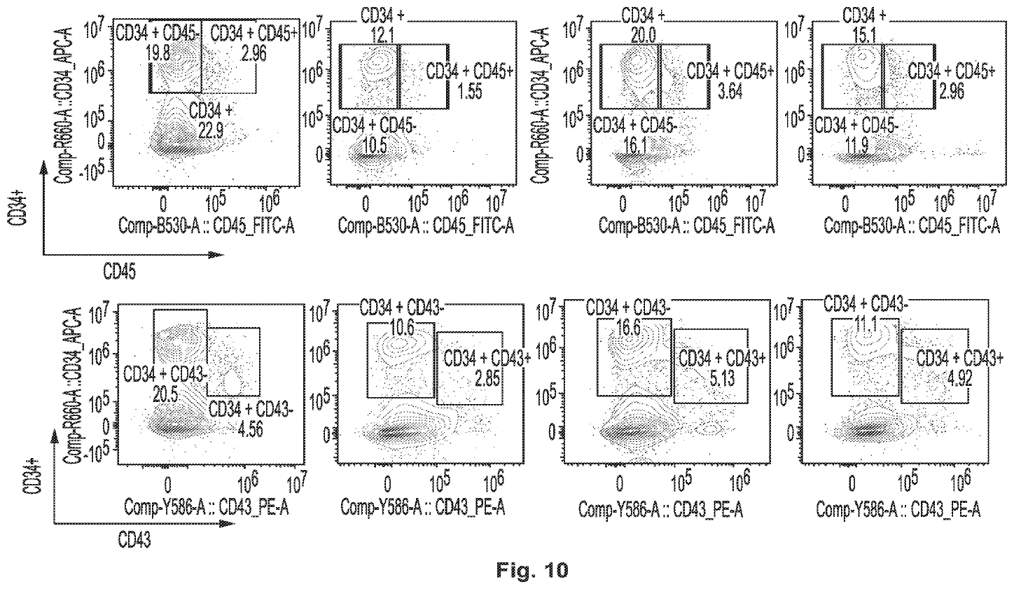

Specifically, the CISH KO INKs, TGFβRII KO INKs, CISH/TGFβRII DKO iNKs were assessed for exemplary phenotypic markers of (i) stem cells (CD34); and (ii) hematopoietic cells (CD43 and CD45) by flow cytometry. Briefly, two rows of embryoid bodies from a 96-well plate for each genotype were harvested for staining. Once a single cell solution was generated using Trypsin and mechanical disruption, the cells were stained for the human markers CD34, CD45, CD31, CD43, CD235a and CD41. As shown in FIG. 9, CISH KO INKs, TGFβRII KO INKs, CISH/TGFβRII DKO iNKs, and iNKs derived from wild-type parental clones (PCS) exhibited lower levels of CD34 relative to control cells, which were purified CD34+HSCs. CD34 expression levels were similar across these iNK cell clones indicating that editing of the iPSCs did not affect differentiation to the CD34+stage. FIG. 10 shows that CISH KO iNKs, TGFβRII KO INKs, CISH/TGFβRII DKO iNKs, and iNKs derived from wild-type parental clones (PCS) exhibited similar surface expression profiles for CD43 and CD45. Thus, iNKs differentiated from edited and unedited iPSCs exhibited similar levels of markers for stem cells and hematopoietic cells, and both differentiated edited and unedited cells exhibited certain NK cell phenotypes based on marker expression profiles.

CISH KO INKs, TGFβRII KO iNKs, CISH/TGFβRII DKO iNKs, iNKs derived from wild-type parental clones (WT), and NK cells derived from peripheral blood (PBNKs) were further assayed to determine their surface expression of CD56, a marker for NK cells. Briefly, cells were harvested on day 39 of differentiation, washed and resuspended in a flow staining buffer containing antibodies that recognize human CD56, CD16, NKp80, NKG2A, NKG2D, CD335, CD336, CD337, CD94, CD158. Cells events were recorded on a NovoCyte Quanteon Flow Cytometer (Agilent) and analyzed using FlowJo (FlowJo, LLC). FIG. 11A shows that iNK cells derived from edited iPSCs exhibited similar CD56+surface expression relative to iNKs derived from unedited iPSC parental clones and PBNK cells (at day 39 in culture). FIG. 11B shows that iNK cells derived from edited iPSCs exhibited similar CD56+ and CD16+surface expression relative to iNKs derived from unedited iPSC parental clones (at day 39 in culture). FIG. 11C shows that iNK cells derived from edited iPSCs exhibited similar CD56+, CD54+, KIR+, CD16+, CD94+, NKG2A+, NKG2D+, NCR1+, NCR2+, and NCR3+surface expression relative to iNKs derived from unedited iPSC parental clones and PBNK cells (at day 39 in culture)

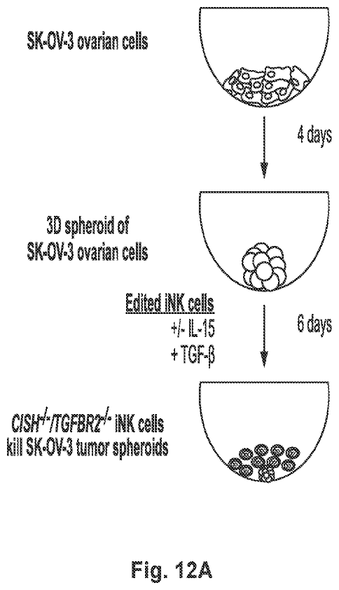

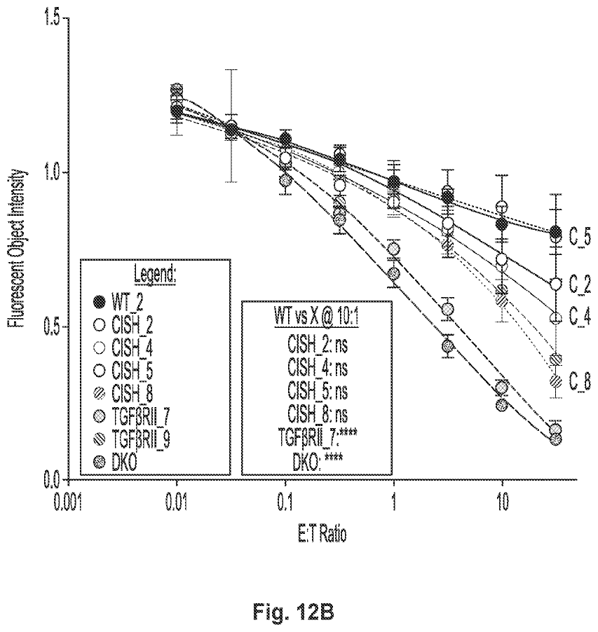

To confirm cell functionality, cells were assessed using a tumor cell cytotoxicity assay on the xCelligence platform. Briefly, tumor targets, SK-OV-3 tumor cells, were plated and grown to an optimal cell density in 96-well xCelligence plates. iNKs were then added to the tumor targets at different E:T ratios (1:4, 1:2, 1:1, 2:1, 4:1 and 8:1) in the presence of TGFβ. FIG. 12C shows that TGFβRII KO and CISH/TGFβRII DKO cells more effectively killed SK-OV-3 cells, as measured by percent cytolysis, relative to unedited iNK cells either in the presence or absence of TGF-β (at E:T ratios of 1:4, 1:2, 1:1, and 2:1).

While iNK cells generated using the alternative method described in FIG. 8B were CD56+ and capable of killing tumor targets in an in vitro cytotoxicity assay, the iNKs did not express many of the canonical markers associated with mature NK cells such as CD16. NKG2A, and KIRs. A K562 feeder cell line is typically used to expand and mature iNKs that are generated by similar differentiation methodologies. After expansion on feeders, the iNKs often express CD16, KIRs and other surface markers indicative of a more mature phenotype. In order to identify a feeder free approach to achieve more mature iNKs with enhanced functionality, an alternative media composition was tested for the stage of differentiation between day 11 and day 39. Instead of culturing cells between day 11 and day 39 in APEL2 (as shown in FIG. 8B), the spin embryoid bodies (SEBs) were cultured in NK MACS® media (MACS Miltenyi Biotec) with 15% human AB serum in the presence of the same cytokines as mentioned above. This protocol is depicted in FIG. 8C. In order to compare the two media compositions, Day 11 SEBs from WT PCS, TGFβRII KO iPSCs, CISH KO iPSCs, and DKO iPSCs were split into two conditions for the second half of the differentiation process, one with APEL2 base and the other with the NKMACS+serum base. At day 39, the cell yield, marker expression, and cytotoxicity levels were assessed. In all cases, the NKMACS+serum condition (depicted in FIG. 8C) outperformed the APEL2 condition (depicted in FIG. 8B). FIG. 8D shows that the NKMACS+serum condition yielded a greater fold expansion at the end of the 39 day process (nearly 300 fold expansion vs 100 fold expansion). When NK marker expression was analyzed by flow cytometry as described above, the iNKs cultured in NKMACS+serum were 34% CD16 positive and exhibited 20% KIR expression while the APEL2 conditions yielded cells that were essentially negative for both markers. This was the case for all genotypes tested. In order to visualize the markers relative to time or condition, flow cytometry data was gated and analyzed in FlowJo and heat maps were constructed (FIGS. 8E and 8F). Samples were first cleaned by gating for live cells (FSC-H vs. LIVE/DEAD™ Fixable Yellow) followed by immune cells (SSC-A vs. FSC-A), singlets (FSC-H vs. FSC-A) and the natural killer cell population (CD56 vs. CD45). The NK population, defined as CD45+56+ cells, was gated and each marker was analyzed along the X-axis in an analysis synonymous to a histogram/count plot (CD16+, CD94+, NKG2A+, NKG2D+, CD335+, CD336+, CD337+, NKp80+, panKIR+). Statistics for the aforementioned markers are visualized with a double-gradient heat map (GraphPad Prism 8) with the key set to the following parameters: black=0, medium intensity 30<x<50, maximum intensity=100. Based on this analysis, the expression kinetics and magnitude across all genotypes were improved by the NKMACS+serum condition. The cells were also assessed in a tumor cell cytotoxicity assay as described previously. The iNKs generated in the NKMACS+serum conditions were capable of killing at a lower E:T ratio than the cells differentiated in APEL2, indicating that the improved NK maturation had a positive impact on the functionality of the cells (FIG. 8G).

Analysis of additional differentiation markers in NKMACS+serum confirmed the presence of CD16 expression. FIG. 11B shows analysis of specific subpopulations (CD45 vs CD56 and CD56 vs CD16) derived from unedited or DKO iPSCs. Additionally, the cell surface marker profile of unedited iNK cells and CISH/TGFβRII DKO iNKs in FIG. 11C confirmed that the NK cell marker profile of the edited iNK cells was similar to that of unedited iNK cells. Taken together, these data show that Cas12a-edited single and double KO iPSC clones differentiate into iNK cells in a similar fashion as unedited iPSC clones, as defined by NK cell markers.

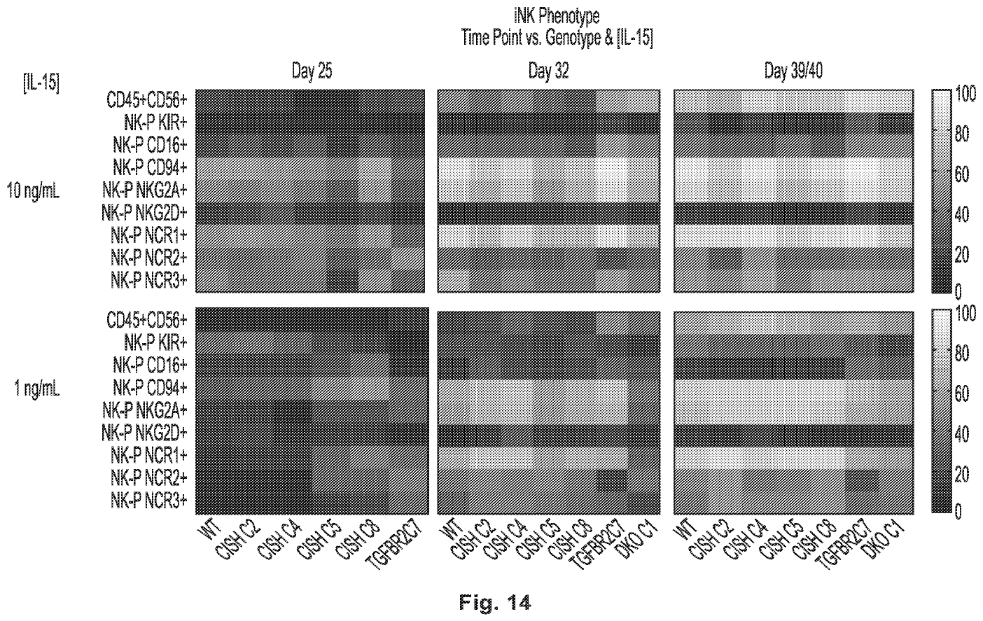

Additionally, certain edited iNK clonal cells (CISH single knockout “CISH_C2, C4, C5, and C8”, TGFβRII single knockout “TGFβRII-C7”, and TGFβRII/CISH double knockout “DKO-C1”), and parental clone iNK cells (“WT”) were cultured in the presence of 1 ng/mL or 10 ng/mL IL-15, and differentiation markers were assessed at day 25, day 32, and day 39 post-hiPSC differentiation. As shown in FIG. 14, surface expression phenotypes (measured as a percentage of the population) culturing in 10 ng/mL IL-15 resulted in a higher proportion of surface expression in the single knockouts, double knockouts, and the parental clonal line.

The edited iNK cells differentiated in NK MACS® medium+serum conditions were assessed for effector function in vitro using a range of molecular and functional analyses. First, a phosphoflow cytometry assay was performed to determine the phosphorylated state of STAT3 (pSTAT3) and SMAD2/3 (pSMAD2/3) in the day 39 iNK cells. CISH KO iNKs exhibited increased pSTAT3 upon IL-15 stimulation (FIG. 11D), and CISH/TGFβRII DKO iNKs exhibited decreased pSMAD2/3 levels upon TGF-β stimulation as compared to unedited iNK cells (FIG. 11E). These data suggest that CISH/TGFβRII DKO iNKs have enhanced sensitivity to IL-15 and resistance to TGF-β mediated immunosuppression. In addition, CISH/TGFβRII DKO iNKs were characterized for IFNγ and TNFα production using a phorbol myristate acetate and Ionomycin (PMA/IMN) stimulation assay. Briefly, cells were treated with 2 ng/ml of PMA and 0.125 μM of Ionomycin along with a protein transport inhibitor for 4 hours. The cells were harvested and stained using a standard intracellular staining protocol. The CISH/TGFβRII DKO INKs produced significantly higher amounts of IFNγ and TFNα when stimulated with PMA/IMN (FIGS. 11F and 11G), providing evidence of enhanced cytokine production following stimulation relative to unedited control iNKs.

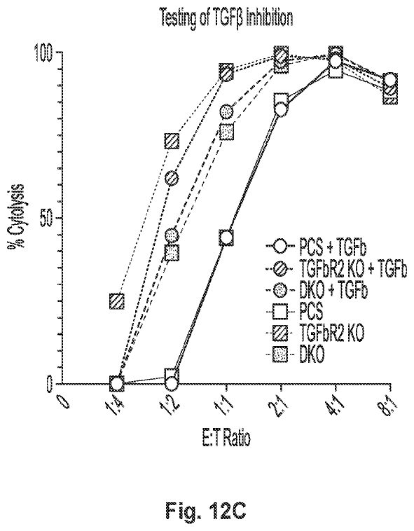

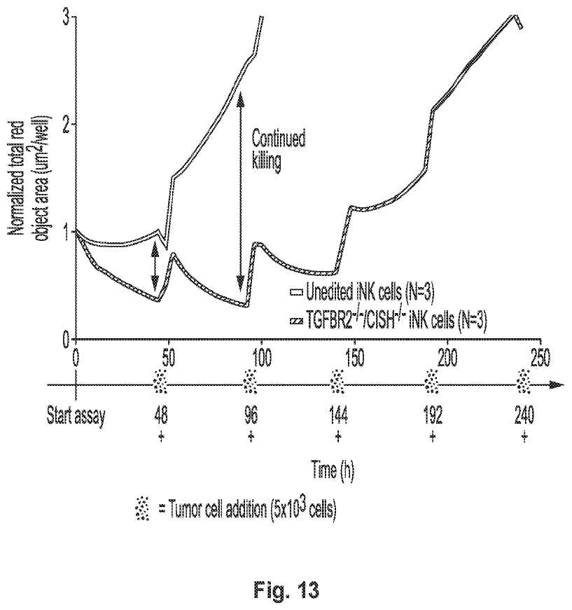

To test iNK tumor cell killing activity, a 3D solid tumor cell killing assay (depicted schematically in FIG. 12A) was utilized. In brief, spheroids were formed by seeding 5,000 NucLight Red labeled SK-OV-3 cells in 96 well ultra-low attachment plates. Spheroids were incubated at 37° C. before addition of effector cells (at different E:T ratios) and 10 ng/ml TGF-β, spheroids were subsequently imaged every 2 hours using the Incucyte S3 system for up to 120 hours. Data shown are normalized to the red object intensity at time of effector addition. Normalization of spheroid curves maintains the same efficacy patterns observed in non-normalized data. Using this assay, the cytotoxicity of iNKs differentiated from four CISH KO iPSC clones, two TGFβRII KO iPSC clones and one CISH/TGFβRII DKO iPSC clone were compared to control iNKs derived from the unedited parental iPSCs. As shown in FIG. 12B, edited iNK cells were capable of reducing the size of SK-OV-3 spheroids more effectively than unedited iNK control cells (averaged data from 6 assays). In particular the CISH/TGFβRII DKO iNK cells reduced the size of SK-OV-3 spheroids to a greater extent than unedited iNK cells at all E:T ratios greater than 0.01, and significantly at E:T ratios of 1 or higher. The TGFβRII KO clone 7 iNKs also exhibited significantly enhanced killing when compared to unedited iNK cells. While a number of single CISH KO clones did not show significant enhancement of killing at the 10:1 E:T ratio, the majority of clones did display a trend towards increased SK-OV-3 spheroid cell killing, with the greatest differential at the highest E:T ratio. To further elucidate the functionality of the edited iNKs, the cells were pushed to kill tumor targets repeatedly over a multiday period, herein described as an in vitro serial killing assay. At day 0 of the assay, 10×10⁶ Nalm6 tumor cells (a B cell leukemia cell line) and 2×10⁵ iNKs were plated in each well of a 96-well plate in the presence of IL-15 (10 ng/ml) and TGF-β (10 ng/ml). At 48 hour intervals, a bolus of 5×10³ Nalm6 tumor cells (a B cell leukemia cell line) was added to re-challenge the iNK population. As shown in FIG. 13, the edited iNK cells (CISH/TGFβRII DKO INK cells) exhibited continued killing of Nalm6 cells after multiple challenges with Nalm6 tumor cells, whereas unedited iNK cells were limited in their serial killing effect. The data supports the conclusion that the CISH and TGFβRII edits result in prolonged enhancement of cell killing.

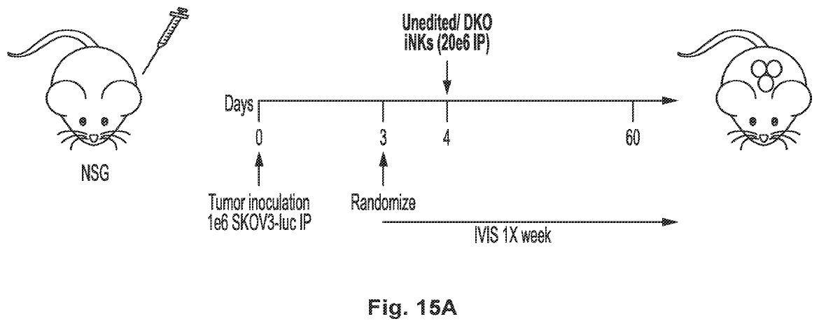

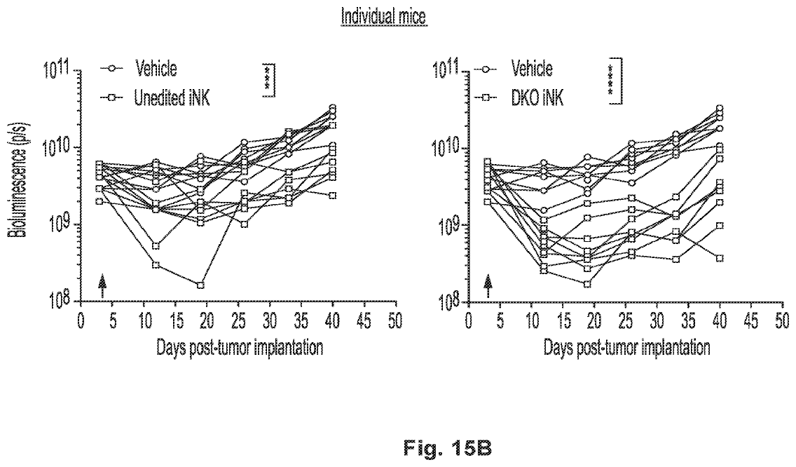

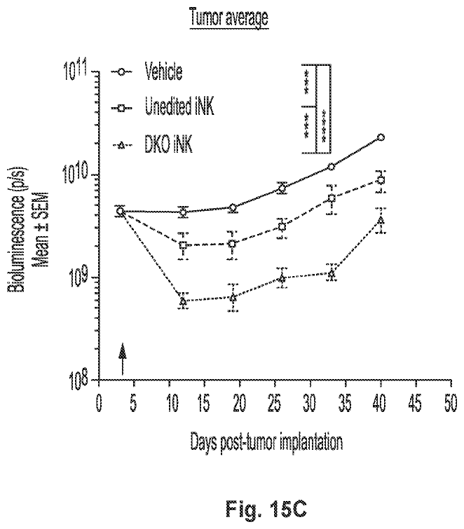

Finally, edited iNK cells (CISH/TGFβRII DKO iNK cells) were assayed for their ability to kill tumor targets in an in vivo model. To this end, an established NOD scid gamma (NSG) xenograft model was utilized in an assay as depicted in FIG. 15A. Briefly, 1×10⁶ SK-OV-3 cells engineered to express luciferase were injected intraperitoneally (IP) at day 0. On day 3, the inoculated mice were imaged using an In vivo imaging system (IVIS) and randomized into 3 groups. The next day (day 4), 20×10⁶ unedited iNKs or CISH/TGFβRII DKO iNKs were administered by IP injection, while a third group was injected with vehicle as a control. Following inoculation of the animals with tumor cells, animals were imaged once a week to measure tumor burden over time. FIG. 15B depicts the bioluminescence of the tumors in the individual mice in the 3 different groups (n=9 in each group), vehicle, unedited iNKs, and CISH/TGFβRII DKO iNKs. The average tumor burden over time for these same animals is depicted in FIG. 15C. A two way anova analysis was performed on the data, and CISH/TGFβRII DKO iNK treated animals had significantly less tumor burden as measured by bioluminescence when compared to animals treated with unedited iNKs (p value: 0.0004). By 10 days post-tumor implantation, mice injected with the CISH/TGFβRII DKO iNKs exhibited a significant reduction in the size of their tumors relative to mice injected with the vehicle controls or the unedited iNKs. The overall reduction in tumor size is seen for several days, and at least until 35 days post-tumor implantation. These data show that the edited DKO iNKs were actively killing tumor cells in this in vivo model.

Overall, these results demonstrate that unedited and CISH/TGFβRII DKO iPSCs can be differentiated into iNK cells exhibiting canonical NK cell markers. Additionally, CISH/TGFβRII DKO iNK cells demonstrated enhanced anti-tumor activity against tumor cell lines derived from both solid and hematological malignancies.

### Example 3: ADORA2A Edited iPSCs Give Rise to Edited iNKs with Enhanced Function

ADORA2A is another target gene of interest, the loss of which is hypothesized to affect NK cell function in a tumor microenvironment (TME). The ADORA2A gene encodes a receptor that responds to adenosine in the TME, resulting in the production of cAMP which functions to drive a number of inhibitory effects on NK cells. We hypothesized that knocking out the function of ADORA2A could enhance iNK cell function. Utilizing a similar approach to the one described in Examples 1 and 2, the PCS iPSC line was edited using a RNP having an engineered Cas12a with three amino acid substitutions (M537R, F870L, and H800A (SEQ ID NO: 1148)) and a gRNA specific to ADORA2A (except that 4 μM RNP was delivered to cells rather than 2 μM RNP). As described in Example 1, the gRNA was generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence. The ADORA2A gRNA sequence is shown in Table 11.

TABLE 11
Guide RNA sequence

| | gRNA | |
|---|---|---|
| | Targeting | |
| | Domain | |
| Target | Sequence | Full Length gRNA Sequence |
| ADORA2A | CCAUCGGCCU | ATGTGTTTTTGTCAAAAGACCTTTTr |
| 4113 | GACUCCCAUG | UrArArUrUrUrCrUrArCrUrCrUr |
| | (SEQ ID | UrGrUrArGrArUrCrCrArUrCrGr |
| | NO: 1159) | GrCrCrUrGrArCrUrCrCrCrArU |
| | | rG (SEQ ID NO: 1160) |

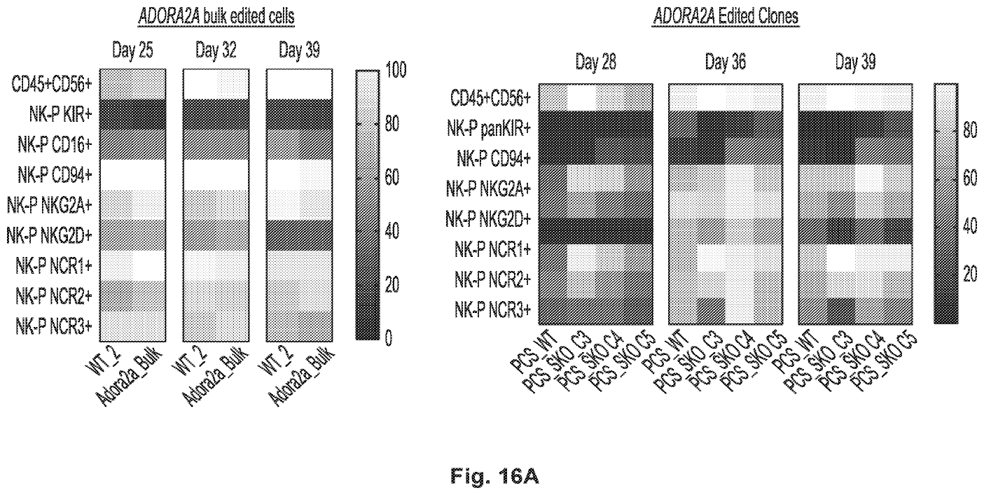

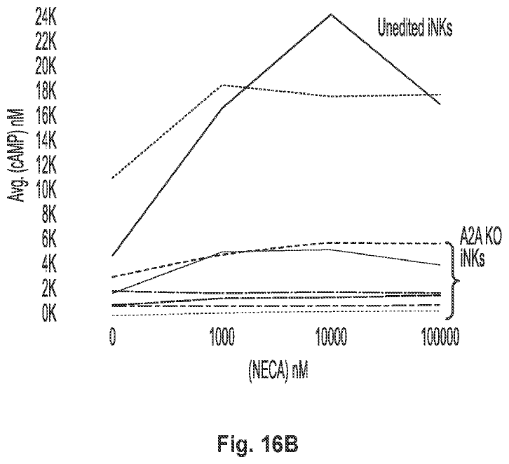

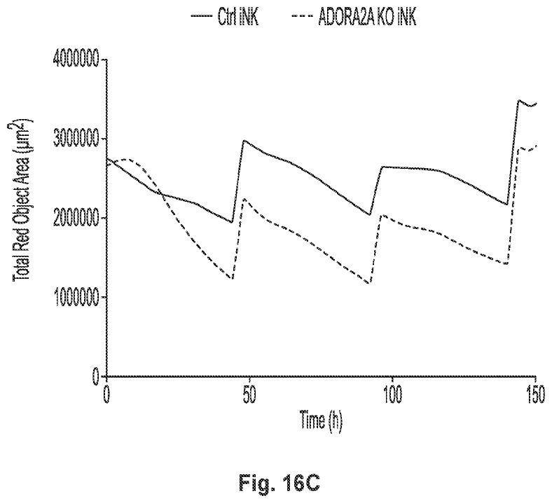

The bulk editing rate by the Cas12a RNP prior to clonal selection was 49% as determined by next-generation sequencing (NGS). Nonetheless, several clones that had both ADORA2A alleles edited were identified, expanded and differentiated. To determine whether an ADORA2A edited iPSC could yield CD45+CD56+ iNKs, both bulk and single ADORA2A KO clones were differentiated using the NKMACS+serum protocol as described in Example 2 (FIG. 8C). As shown in FIG. 16A, edited iPSCs differentiated to iNKs with similar NK cell marker expression compared to unedited control iPSCs.

To confirm that Cas12a-mediated ADORA2A editing resulted in a functional deletion of the gene, cAMP accumulation in response to treatment with 5′-N-ethylcarboxamide adenosine (“NECA”, a more stable adenosine analog that acts as an ADORA2A agonist) was assessed in both the edited and unedited control iNKs. Edited cells with a functional knockout of ADORA2A would not be expected to accumulate as much CAMP in the cells in response to NECA relative to cells with functional ADORA2A. Briefly, iNK cells were treated with varying concentrations of NECA for 15 minutes. The iNK cells were then lysed, and the CAMP in the lysate was then measured using a CisBio CAMP kit. As shown in FIG. 16B, unedited iNKs had increased levels of cAMP accumulation as the concentration of NECA was increased (n=2). Conversely, the ADORA2A (“A2A KOs”) KO iNKs showed minimal production of cAMP at increasing concentrations of NECA, indicating that the Cas12a-induced edits functionally knocked out ADORA2A function. The bulk iNKs (top two A2A KO iNK lines in FIG. 16B) exhibited slightly higher levels of cAMP than the selected ADORA2A KO clones (lower four A2A KO iNK lines in FIG. 16B), as would be expected from the lower editing rates in the bulk population. Based on this molecular evidence of functional ablation of ADORA2A, the iNKs would be expected to be resistant to the inhibitory effects of adenosine in a tumor microenvironment.

The ADORA2A KO iNKs were also tested in an in vitro NALM6 serial killing assay as described in Example 2, with one main difference: 100 μM of NECA was added in place of TGFβ. The ADORA2A KO iNKs exhibited enhanced serial killing relative to the wild type iNKs in the presence of NECA, indicating that the ADORA2A KO iNKs were resistant to NECA inhibition (FIG. 16C). As a result, the ADORA2A KO iNK cells would be expected to have improved cytotoxicity against tumor cells in the presence of adenosine in the TME relative to unedited iNK cells.

### Example 4: Generation of CISH/TGFβRII/ADORA2A triple edited (TKO) iPSCs and the characterization of differentiated TKO iNKs

In order to generate CISH, TGFβRII, and ADORA2A triple edited (TKO) iPSCs, two approaches were taken; 1) two step editing in which the CISH/TGFβRII DKO (CR) iPSC clone described in Examples 1 and 2 was edited at the ADORA2A locus via electroporation with an ADORA2A targeting RNP (as described in Example 3), and 2) simultaneous editing of PCS iPS cells with all 3 RNPs, one for each target gene. Both strategies utilized the editing protocol briefly described in Example 1. In the case of simultaneous editing, the total RNP concentration was 8 μM (Cish:2 μM+ TGFβRII:2 μM+ADORA2A:4 μM). Regardless of the approach, cells were plated, expanded and colonies were picked as described above. Using NGS to analyze gDNA harvested from the iPSCs, it was determined that the bulk editing rates were 96.70%, 97.17%, and 90.16% for CISH, TGFβRII and ADORA2A, respectively, when all target genes were edited simultaneously. Picked colonies that had Insertions and/or Deletions (InDels) at all 6 alleles were selected for further analysis.

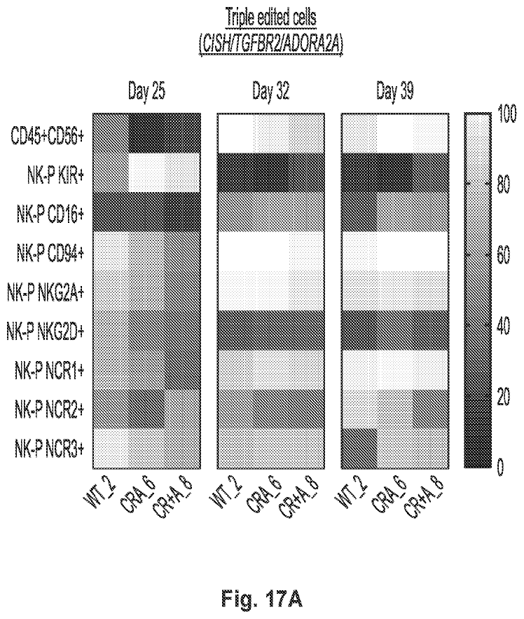

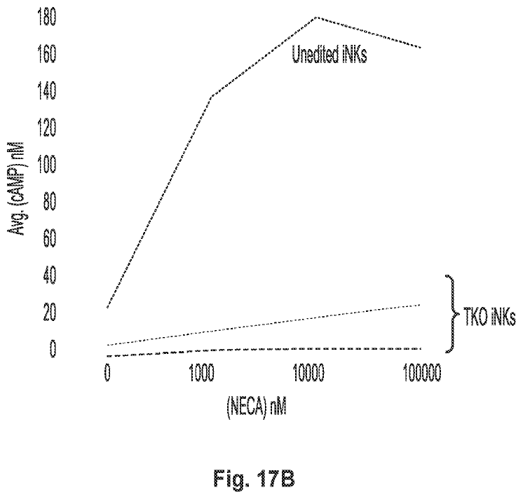

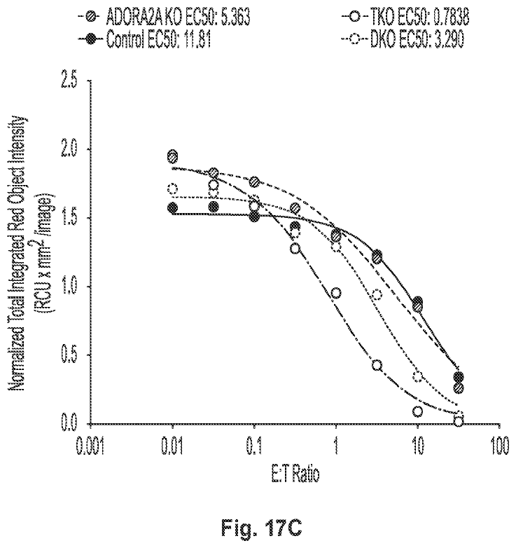

Similar to the analysis described in Example 1, unedited iPSCs and the edited iPSCs were differentiated to iNKs using the NK MACS+Serum condition (described in FIG. 8C) and assessed by flow cytometry at different time points, including at day 25, day 32, and day 39 in culture. As shown in FIG. 17A, analysis of the different NK surface markers revealed no major differences between clones that were generated by the two-step editing method (CR+A 8) or the simultaneous editing method (CRA 6). Both TKO clones (CR+A 8 and CRA 6) showed similar expression profiles to the unedited iNKs (Wt) at each time point. When the TKO iNK cells were analyzed for their responsiveness to NECA (as described in Example 3), both TKO iNKs had little to no cAMP accumulation (FIG. 17B), demonstrating that ADORA2A was functionally knocked out. By contrast, the unedited iNKs demonstrated a NECA dose dependent increase in cAMP (FIG. 17B). These results indicate that the TKO iNKs would be expected to be resistant to the inhibitory effects of adenosine in the TME. Finally, the CISH/TGFβRII/ADORA2A TKO iNKs were assessed alongside CISH/TGFβRII DKO iNKs, ADORA2A single KO (SKO) iNKs, and unedited iNKs in a 3D tumor cell killing assay. This assay was performed as described in Example 2 with IL-15 and TGFβ but without NECA. Interestingly, both the TKO (CRA6) and DKO (CR) iNKs outperformed the unedited iNKs in killing the tumor cells, indicating that both multiplex edited iNKs have enhanced function over unedited control cells (FIG. 17C). These results show that knocking out ADORA2A does not negatively affect the ability of iNKs having CISH and TGFβRII KOs to kill tumor spheroid cells.

### Example 5: Selection of CISH, TGFβRII, ADORA2A, TIGIT, and NKG2A Targeting gRNAs

The cutting efficiency of CISH, TGFβRII, ADORA2A, TIGIT, and NKG2A Cas 12a guide RNAs were further tested. Guide RNAs were screened by complexing commercially synthesized gRNAs with Cas12a in vitro and delivering gRNA/Cas12a ribonucleoprotein (RNP) to IPSCs via electroporation. The iPSCs were edited using a RNP having an engineered Cas 12a with three amino acid substitutions (M537R, F870L, and H800A (SEQ ID NO: 1148)). The gRNAs were generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence. Table 12 provides the targeting domains of the guide RNAs that were tested for editing activity.

TABLE 12
Guide RNA sequences

| Target | gRNA Targeting Domain Sequence |
|---|---|
| TGFBRII | UGAUGUGAGAUUUUCCACCUG (SEQ ID NO: 1161) |
| CISH | ACUGACAGCGUGAACAGGUAG (SEQ ID NO: 1162) |
| ADORA2A | CCAUCGGCCUGACUCCCAUGC (SEQ ID NO: 1163) |
| ADORA2A | CCAUCACCAUCAGCACCGGGU (SEQ ID NO: 1164) |
| ADORA2A | CCUGUGUGCUGGUGCCCCUGC (SEQ ID NO: 1165) |
| TIGIT | UGCAGAGAAAGGUGGCUCUAU (SEQ ID NO: 1166) |
| TIGIT | UCUGCAGAAAUGUUCCCCGUU (SEQ ID NO: 1167) |
| TIGIT | UAGGACCUCCAGGAAGAUUCU (SEQ ID NO: 1168) |
| NKG2A | GCAACUGAACAGGAAAUAACC (SEQ ID NO: 1169) |
| NKG2A | GUUGCUGCCUCUUUGGGUUUG (SEQ ID NO: 1170) |
| NKG2A | AAGGGAAUGACAAAACCUAUC (SEQ ID NO: 1171) |

In brief, 100,000 iPSCs/well were transfected with the RNP of interest, cells were incubated at 37° C. for 72 hours, and then harvested for DNA characterization. iPSCs were transfected with gRNA/Cas12a RNPs at various concentrations. The percentage editing events were determined for eight different RNP concentrations ranging from negative control (0 mM), to 8 mM.

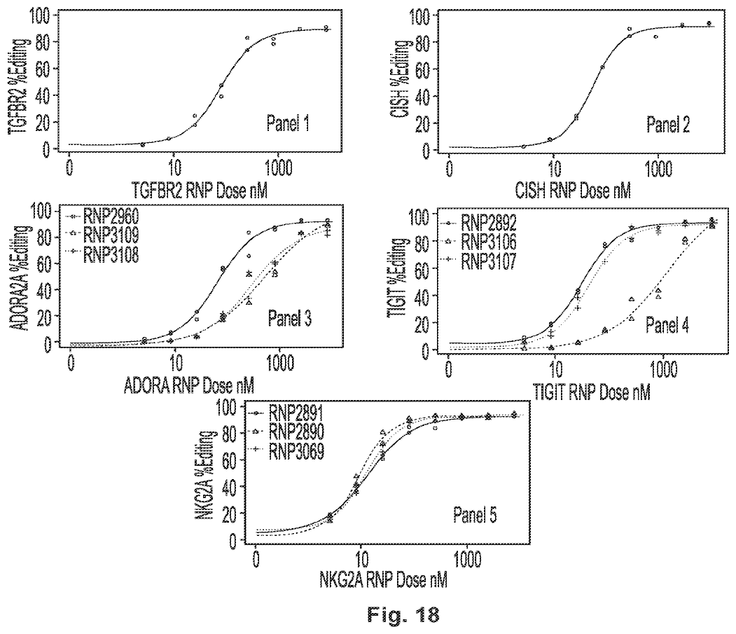

As shown in FIG. 18 panel 1, the TGFβRII gRNA (SEQ ID NO: 1161) exhibited an EC50 of ˜79 nM RNP. As shown in FIG. 18 panel 2, the CISH gRNA (SEQ ID NO: 1162) exhibited an EC50 of ˜50 nM RNP. As shown in FIG. 18 panel 3, an ADORA2A gRNA (SEQ ID NO: 1163) included in RNP2960 exhibited an EC50 of ˜63 nM RNP, while an ADORA2A gRNA (SEQ ID NO: 1164) included in RNP3109, or gRNA (SEQ ID NO: 1165) included in RNP3108 exhibited EC50 values of ˜493 nM and ˜280 nM RNP respectively. As shown in FIG. 18 panel 4, a TIGIT gRNA (SEQ ID NO: 1166) included in RNP2892 exhibited an EC50 of ˜29 nM RNP, while a TIGIT gRNA (SEQ ID NO: 1167) included in RNP3106, or gRNA (SEQ ID NO: 167) included in RNP3107 exhibited EC50 values of ˜1146 nM and ˜40 nM RNP respectively. As shown in FIG. 18 panel 5, a NKG2A gRNA (SEQ ID NO: 1169) included in RNP19142 exhibited an EC50 of ˜8 nM RNP, while a NKG2A gRNA (SEQ ID NO: 1170) included in RNP3069, or gRNA (SEQ ID NO: 1171) included in RNP2891 exhibited EC50 values of ˜12 nM and ˜13 nM RNP respectively.

### Example 6: Selection by Essential Gene Knock-In

Exemplary selection systems illustrated in FIGS. 19A, 19B, and 19C were tested at the essential gene GAPDH in iPSCs using an RNP comprising AsCpf1 (SEQ ID NO: 1148), and a guide RNA (RSQ22337 (AUCUUCUAGGUAUGACAACGA, SEQ ID NO: 1178)), resulting in a double-strand break towards the 5′ end of the last exon of GAPDH (exon 9). RSQ22337 was determined to be highly specific to GAPDH and have minimal off-target sites in the genome (data not shown). GAPDH was thus considered a good exemplary candidate target gene for the cargo integration and selection methods described herein, at least in part because there was at least one highly specific gRNA targeting a terminal exon capable of mediating highly efficient RNA-guided cleavage.

The CRISPR/Cas nuclease and guide RNA were introduced into cells by nucleofection (electroporation) of a ribonucleoprotein (RNP) according to known methods. The cells were also contacted with a double stranded DNA donor template (e.g., a dsDNA plasmid) that included a knock-in cassette comprising in 5′-to-3′ order, a 5′ homology arm approximately 500 bp in length (comprising a portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence for a “Cargo” sequence, a stop codon and polyA signal sequence, and a 3′ homology arm approximately 500 bp in length (comprising a coding portion of exon 9 including a stop codon, the 3′ exonic region of exon 9, and a portion of the downstream intergenic sequence) (as shown in FIG. 19B). The 5′ and 3′ homology arms flanking the knock-in cassette were designed to correspond to sequences surrounding the RNP cleavage site.

As shown schematically in FIG. 19C, NHEJ-mediated creation of indels in cells that are edited by the DNA nuclease but not successfully targeted by the DNA donor template, produce a non-functional version of GAPDH which is lethal to the cells. This knock-out is “rescued” in cells that are successfully targeted by the DNA donor template by correct integration of the knock-in cassette, which restores the GAPDH coding region so that a functioning gene product is produced, and positions the P2A-Cargo sequence in frame with and downstream (3′) of the GAPDH coding sequence. These cells survive and continue to proliferate. Cells that are not edited by the DNA nuclease also continue to proliferate but are expected to represent a very small percentage of the overall cell population, if, as in this case, the editing efficiency of the nuclease in combination with the gRNA is high (data not shown) and results in creation of a non-functional protein. The editing results for RSQ22337 likely underestimate the actual editing efficiency of the guide due to cell death within the population of edited cells.

An experiment was then conducted to test the mechanism of the selection system described above by confirming that edited cells containing a successfully knocked-in cargo gene would be more efficiently selected for using a gRNA targeting a protein-coding exonic portion of GAPDH rather than a gRNA targeting an intron. FIG. 19E compares the knock-in efficiency of a GFP-encoding “cargo” knock-in cassette at the GAPDH locus when using a gRNA that mediates cleavage within an intron (RSQ24570 (CUGGUAUGUGGCUGGGGCCAG; SEQ ID NO: 1200) binds to the exon 8-intron 9 junction, leading to Cas12a-mediated cleavage within intron 8) relative to a gRNA specific for an exon (RSQ22337 (SEQ ID NO: 1178), targeting the intron 8-exon 9 junction, leading to Cas12a-mediated cleavage within exon 9). Rescue dsDNA plasmid PLA1593 comprising the reporter “cargo” GFP was nucleofected into iPSCs with an RNP (comprising Cas12a and RSQ22337) targeting GAPDH as described above, while dsDNA plasmid PLA1651 comprising a donor template sequence specific for this insertion site (data not shown) was nucleofected with an RNP comprising Cas12a and RSQ24570. The homology arms of each plasmid were designed to mediate HDR based on the target site of each gRNA. Knock-in was visualized using microscopy and was measured using flow cytometry (FIG. 19E). Knock-in efficiency was significantly higher when using a gRNA and associated knock-in cassette that cleaves at an exonic coding region (exon 9) when compared to an intronic region (intron 8). FIG. 19E shows that 95.6% of cells electroporated with RSQ22337 and the GFP-encoding “cargo” knock-in cassette (e.g., PLA1593; comprising donor template SEQ ID NO: 1198) expressed GFP compared to only 2.1% of cells electroporated with RSQ24570 and a GFP-encoding “cargo” knock-in cassette. The results depicted in FIG. 19E are striking, as while the measured editing efficiency (as determined by indel generation frequency 72 hours post-transfection, data not shown) of RSQ24570 is higher than that of RSQ22337, the proportion of cells rescued by the knock-in construct targeting the coding exonic region are significantly higher.

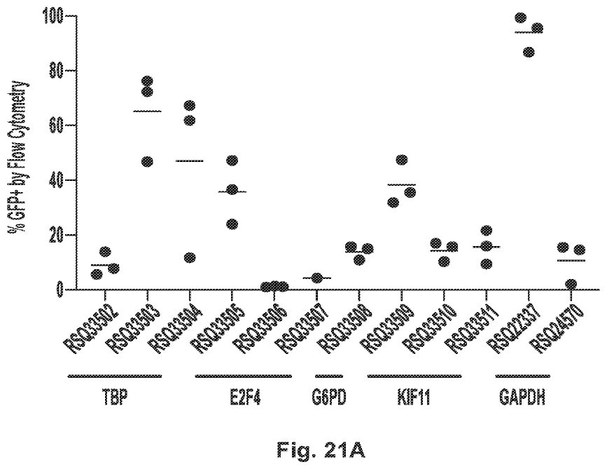

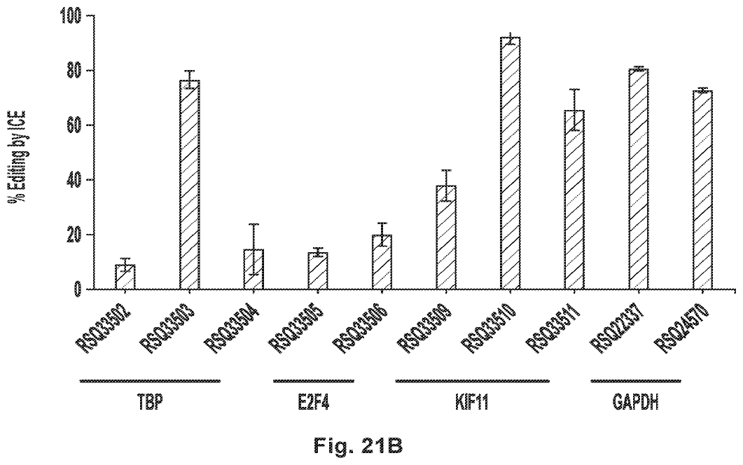

In an additional set of experiments, iPSCs were contacted with an RNP comprising AsCpf1 (SEQ ID NO: 1148), and RSQ22337 (SEQ ID NO: 1178) or RSQ24570 (SEQ ID NO: 1200), along with either the PLA1593 (comprising donor template SEQ ID NO: 1198) or the PLA1651 (data not shown) double stranded DNA donor template plasmid, respectively, as described above. Flow cytometry was performed 7 days following nucleofection to detect GFP expression and help determine to what extent each plasmid mediated donor template and knock-in cassette was integrated successfully at its respective GAPDH target site. The GAPDH specific results in FIG. 21A show that cells nucleofected with the RNP containing RSQ22337 exhibited a much higher amount of GFP expression relative to cells nucleofected with RSQ24750, showing that most cells express GFP at day 7 following electroporation. This suggests that the GFP-encoding knock-in cassette integrated successfully at high levels within the RSQ22337-transfected cells. Cells nucleofected with RNPs containing RSQ24750 displayed much lower GFP expression, indicating that the knock-in cassette did not integrate successfully in most of these cells (FIG. 21A). The GAPDH results of FIG. 21B show that use of RSQ22337 resulted in about 80% editing as measured using genomic DNA 48 hours following RNP transfection, while RSQ24570 resulted in about 75% editing as measured using genomic DNA 48 hours following RNP transfection. The high editing of RSQ22337 correlated well with the high GFP expression level depicted in FIG. 21A; however, the high editing of RSQ24750 correlated poorly with the low GFP expression level depicted in FIG. 21A.

As shown in FIGS. 21A and 21B, similar experiments were conducted at additional loci including TBP, E2F4, G6PD, and KIF11. gRNA sequences utilized for these various experiments are listed in Table 15.

TABLE 15
guide RNA sequences

| SEQ | | gRNA targeting | |
|---|---|---|---|
| ID | | domain sequence | |
| NO: | Name | (RNA) | Gene-Location |
| 1201 | RSQ22336 | UGAGCCAGCCACCAGAGGGCG | GAPDH-Intron 8 |
| 1178 | RSQ22337 | AUCUUCUAGGUAUGACAACGA | GAPDH-Intron 8/ |
| | | | Exon 9 |
| | | | (cut site in |
| | | | exon 9) |
| 1202 | RSQ22338 | GCUACAGCAACAGGGUGGUGG | GAPDH-Exon 9 |
| 1203 | RSQ24559 | CCAUAAUUUCCUUUCAAGGUG | GAPDH-Intron 7 |
| 1204 | RSQ24560 | CUUUCAAGGUGGGGAGGGAGG | GAPDH-Intron 7 |
| 1205 | RSQ24561 | AAGGUGGGGAGGGAGGUAGAG | GAPDH-Intron 7 |
| 1206 | RSQ24562 | GCAGACCACAGUCCAUGCCAU | GAPDH-Exon 8 |
| 1207 | RSQ24563 | CAGACCACAGUCCAUGCCAUC | GAPDH-Exon 8 |
| 1208 | RSQ24564 | CCGGAGGGGCCAUCCACAGUC | GAPDH-Exon 8 |
| 1209 | RSQ24565 | UAGACGGCAGGUCAGGUCCAC | GAPDH-Exon 8 |
| 1210 | RSQ24566 | CUAGACGGCAGGUCAGGUCCA | GAPDH-Exon 8 |
| 1211 | RSQ24567 | UCUAGACGGCAGGUCAGGUCC | GAPDH-Exon 8 |
| 1212 | RSQ24568 | GCAGGUUUUUCUAGACGGCAG | GAPDH-Exon 8 |
| 1213 | RSQ24569 | UCAAGCUCAUUUCCUGGUAUG | GAPDH-Exon 8 |
| 1200 | RSQ24570 | CUGGUAUGUGGCUGGGGCCAG | GAPDH-Exon 8/ |
| | | | Intron 8 |
| | | | (cut site in |
| | | | intron 8) |
| 1214 | RSQ24571 | AGAGCCAGUCUCUGGCCCCAG | GAPDH-Intron 8 |
| 1215 | RSQ24572 | AAGAGCCAGUCUCUGGCCCCA | GAPDH-Intron 8 |
| 1216 | RSQ24573 | UAAGAGCCAGUCUCUGGCCCC | GAPDH-Intron 8 |
| 1217 | RSQ24574 | CUGAGCCAGCCACCAGAGGGC | GAPDH-Intron 8 |
| 1218 | RSQ24575 | UCUGAGCCAGCCACCAGAGGG | GAPDH-Intron 8 |
| 1219 | RSQ24576 | CAUCUUCUAGGUAUGACAACG | GAPDH-Exon 9 |
| 1220 | RSQ33502 | AAAUGCUUCAUAAAUUUCUGC | TBP-Isoform 1 exon |
| | | | 8; isoform 2 exon 7 |
| 1221 | RSQ33503 | UGCUCUGACUUUAGCACCUAA | TBP-Isoform 1 exon |
| | | | 8; isoform 2 exon 7 |
| 1222 | RSQ33504 | AAAACAUCUACCCUAUUCUAA | TBP-Isoform 1 exon |
| | | | 8; isoform 2 exon 7 |
| 1223 | RSQ33505 | CCCCUCUGCUUCGUCUUUCUC | E2F4-Exon 10 |
| 1224 | RSQ33506 | UCCACCCCCGGGAGACCACGA | E2F4-Exon 10 |
| 1225 | RSQ33507 | AUGUGCCUGUUCUCAACCUCU | E2F4-Exon 10 |
| 1226 | RSQ33508 | CAGUAUGAGGGCACCUACAAG | G6PD-1-Exon 13 |
| 1227 | RSQ33509 | CCGCCUUAAAUCCACAGCAUA | KIF11-Intron 21/ |
| | | | Exon 22 |
| 1228 | RSQ33510 | UAACCAAGUGCUCUGUAGUUU | KIF11-Exon 22 |
| 1229 | RSQ33511 | GACCUCUCCAGUGUGUUAAUG | KIF11-Exon 22 |

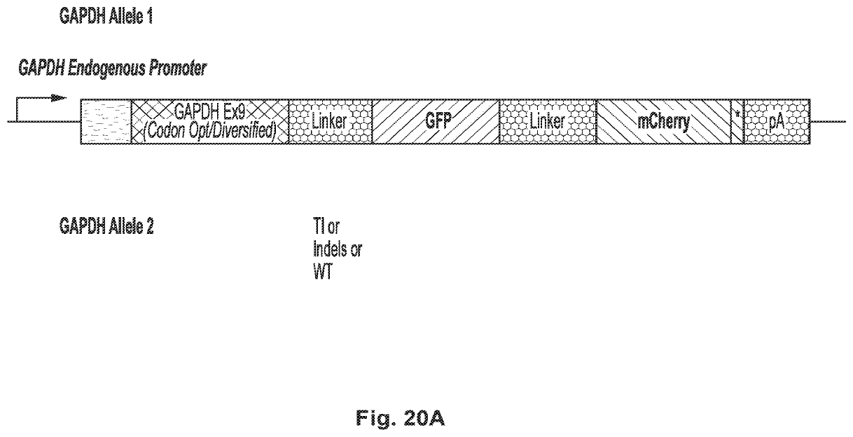

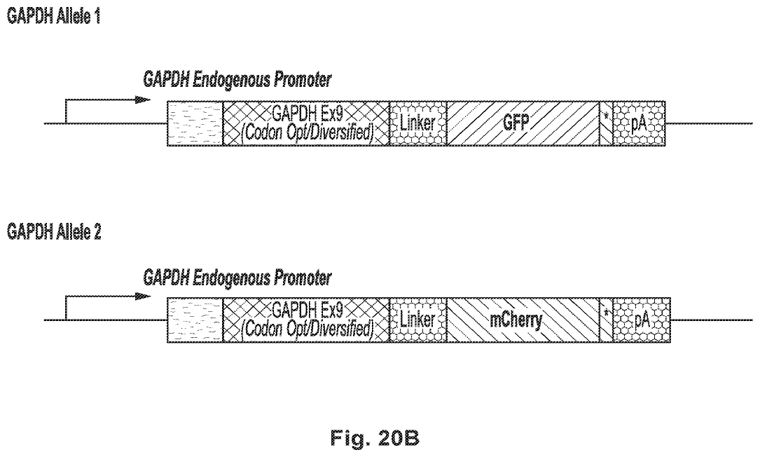

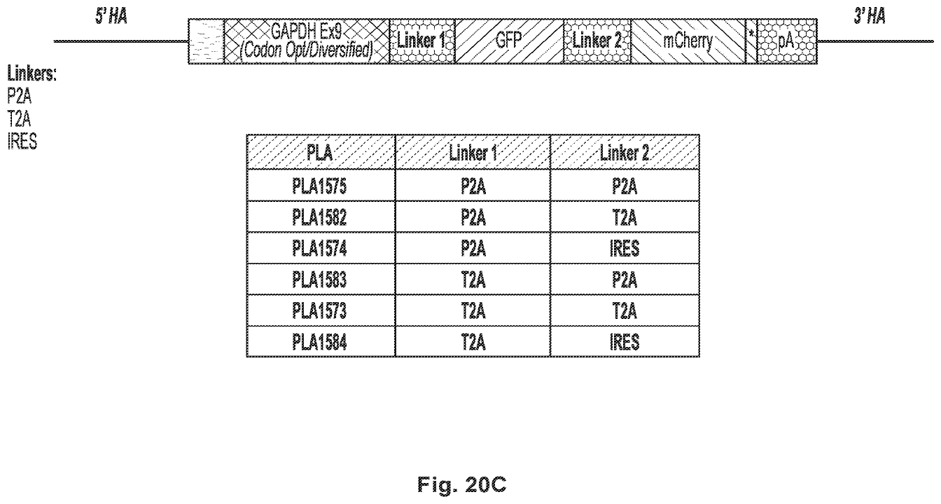

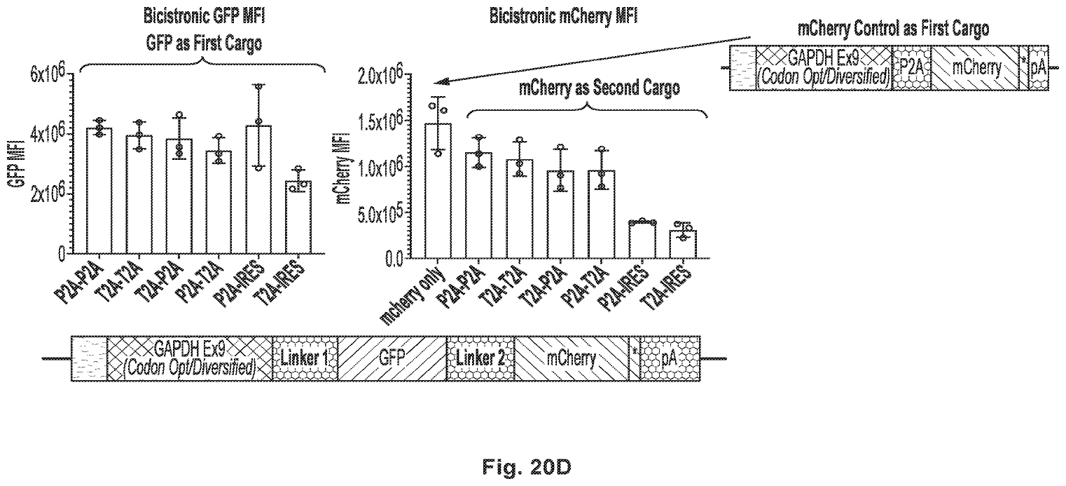

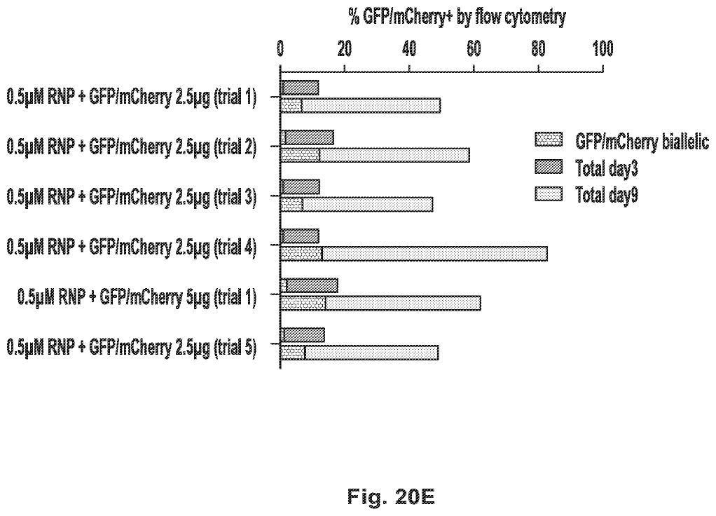

In some cases, it is desirable to use selection and cargo knock in strategies disclosed herein to efficiently produce and isolate an edited cell containing two or more different exogenous coding sequences, e.g., two or more different exogenous genes, integrated into a single essential gene locus, such as, e.g., the GAPDH locus. FIGS. 20A and 20B shows two different strategies for introducing two or more different exogenous coding regions into an essential gene locus. FIG. 20A shows a first exemplary strategy wherein a multi-cistronic knock-in cassette, e.g., a bi-cistronic knock-in cassette containing two or more coding regions (GFP and mCherry in FIG. 20A), separated by linkers (e.g., T2A, P2A, and/or IRES; see table 14), is inserted into one or both of the alleles of the essential gene, e.g., GAPDH. FIG. 20B shows a second exemplary strategy (a bi-allelic insertion strategy) wherein two knock-in cassettes comprising different cargo sequences (e.g., different exogenous genes, such as GFP and mCherry in FIG. 20B) are inserted into separate alleles of the essential gene locus, e.g., GAPDH.

Experiments were conducted to test the integration strategy depicted in FIG. 20A, and to determine whether the use of different combinations of linkers in the knock-in cassette could affect the expression of the cargo sequences. An RNP comprising Cas12a and RSQ22337 (targeting the GAPDH locus, as described above) was nucleofected into iPSCs with one of six different plasmids (PLA) containing a bi-cistronic knock-in cassette comprising “cargo” sequences encoding GFP and mCherry (PLA1573, PLA1574, PLA1575, PLA1582, PLA1583, and PLA1584, as depicted in FIG. 20C; comprising donor templates, data not shown). GFP was the first cargo and mCherry was the second cargo in each of these constructs. Each of the tested plasmids contained a different combination of linkers between the coding sequences (Linkers 1 and 2, as depicted in FIG. 20C). PLA1573 contained T2A and T2A as linkers 1 and 2, respectively; PLA1574 contained P2A and IRES as linkers 1 and 2, respectively; PLA1575 contained P2A and P2A as linkers 1 and 2, respectively; PLA1582 contained P2A and T2A as linkers 1 and 2, respectively; PLA1583 contained T2A and P2A as linkers 1 and 2, respectively; and PLA1584 contained T2A and IRES as linkers 1 and 2, respectively. Various knock-in cassette integration events at the GAPDH locus were analyzed by brightfield and fluorescent microscopy, and edited iPSCs nine days following nucleofection with exemplary plasmids PLA1582, PLA1583, and PLA1584 all exhibited detectable GFP and mCherry expression (data not shown).

FIG. 20D quantifies the fluorescence levels of GFP and mCherry in the iPSCs nucleofected with the various plasmids described in FIG. 20A containing the bi-cistronic knock-in cassettes with the different described linker pairs (PLA1575, PLA1582, PLA1574, PLA1583, PLA1573, and PLA1584). In each of these bi-cistronic constructs, GFP was always the first cargo and mCherry was always the second cargo. A plasmid containing a knock-in cassette with mCherry as a sole “cargo” (as depicted in FIG. 20D) was also tested as a control. The data show that the expression levels of GFP, as the first cargo, were similar between bicistronic constructs and consistently higher than the expression levels of mCherry, the second cargo. Cells containing the control knock-in cassette containing mCherry as the sole cargo exhibited the highest mCherry expression, suggesting that it is possible to vary (e.g., reduce) expression of a cargo by placing it as the second cargo in a bicistronic cassette. In addition, FIG. 20D shows that placement of an IRES linker immediately prior to the second cargo coding sequence resulted in lower expression of the second cargo when compared to the placement of a P2A or T2A linker prior to the second cargo coding sequence. Thus, the results show that it is possible to differentially modulate (i.e., increase or decrease) the expression of two cargo coding sequences from a multicistronic knock-in cassette by varying the order of the cargos in the cassette (placing a cargo as the first cargo for higher expression, or as the second cargo for lower expression) and by placing particular linkers (P2A or T2A for higher expression; IRES for lower expression) upstream of each of the cargos.

An experiment was conducted to test the bi-allelic integration strategy depicted in FIG. 20B. An RNP containing Cas12a and RSQ22337 (targeting the GAPDH locus, as described above) was nucleofected into iPSCs with two different plasmids. One plasmid contained a knock-in cassette containing a GFP coding sequence as the cargo, and the second plasmid contained a knock-in cassette containing an mCherry coding sequence as the cargo (as depicted in FIG. 20B). Nucleofected iPSCs were also assessed using flow cytometry, and gating showed that a high percentage, approximately 15%, of the nucleofected cells expressed GFP and mCherry, suggesting that the GFP knock-in cassette and the mCherry knock-in cassette were each integrated into an allele of GAPDH (data not shown). Approximately 41% of the nucleofected cells expressed mCherry and approximately 36% of the nucleofected cells expressed GFP.

An additional experiment was conducted to test biallelic insertion of GFP and mCherry in populations of iPSCs. The iPSC populations were transformed as described above. The cells were nucleofected with 0.5 μM RNPs comprising Cas12a and RSQ22337 (targeting the GAPDH locus, as described above), and 2.5 μg of donor template (5 trials) or 5 μg of donor template (1 trial), and then sorted 3 or 9 days following nucleofection. FIG. 20E provides the flow cytometry analysis results from these trials. The larger bar at each time point (day 3 or day 9) in FIG. 20E represents the total percentage of the cells in each population that positively express at least one cargo, e.g., at least one allele of GFP and/or at least one allele of mCherry cargo. The smaller bar at each time point shows the percentage of cells in each population that express both GFP and mCherry and therefore represents cells with GFP/mCherry biallelic integration. These results showed that approximately 8-15% percent of the transformed cells in each population displayed a biallelic GFP/mCherry insertion phenotype at nine days following transformation.

### Example 7: Generation and Characterization of B2M Knockout and/or CD47/HLA-E/HLA-G Knock-In iPSCs and iPSC-Derived iNKs

To protect allogeneic iNKs from recipient immune system rejection, HLA class I expression was eliminated by knocking out beta-2 microglobulin (B2M), a universal component of all HLA class I molecules, using methods as described herein. In brief, iPSCs were created as described in Example 1; these cells were then transformed with an RNP complex comprising AsCpf1 (SEQ ID NO: 1148), and a guide RNA targeting B2M (with a targeting domain sequence of AGTGGGGGTGAATTCAGTGTA (as presented as DNA); SEQ ID NO: 412). Cells were allowed to recover and were expanded as described in Example 1.

Removal of B2M can minimize host T-cell mediated rejection; however, loss of HLA antigens may increase susceptibility to iNK cell killing by a recipient's endogenous natural killer (NK) cells. In order to overcome such a rejection, an Allo shield comprising one or more HLA-E, HLA-G, or CD47 peptides was transgenically overexpressed to reduce B2M KO iNK rejection by recipient NK cells. HLA-G is recognized by inhibitory receptors ILT2 (found on some NK cells) and KIR2DL4 (found on all NK cells); HLA-E is recognized by inhibitory receptor NKG2A (found on most NK cells) and activating receptor NKG2C (found on few NK cells, but expanded in CMV+individuals); while CD47 is recognized by inhibitory receptor SIRPa (found on some activated NK cells).

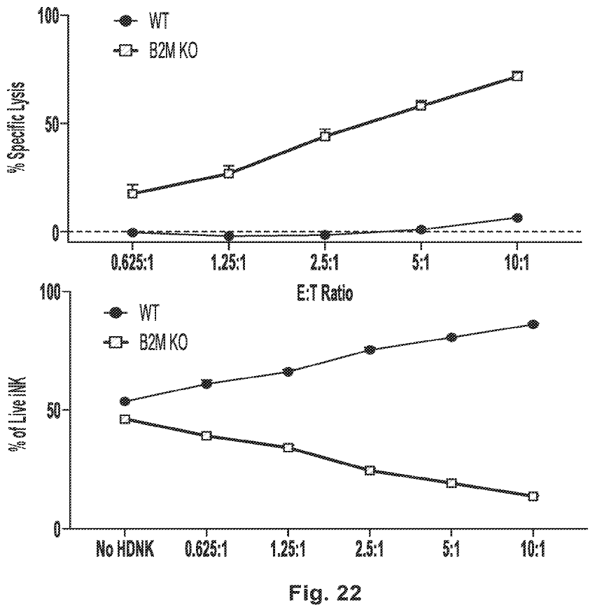

To assess HDNK specific killing of B2M KO iNKs in comparison to WT iNKs, a lineage trace assay was utilized. In brief, B2M KO iNK cells (e.g., ˜25,000 cells) were stained with cell trace violet, WT iNK cells (e.g., ˜25,000 cells) were stained with CSFE, and WT HDNK cells (˜31,000 to ˜500,000 dependent upon E:T ratio) remained undyed, these three cell populations were mixed together and co-cultured overnight (e.g., 16 hours). Post-culturing concentrations of the various cell types were compared to the pre-culturing concentrations using flow-cytometry. As shown in FIG. 22, edited B2M KO iNKs exhibited greater specific lysis and greater cell death when compared to WT iNKs when measured at E:T ratios ranging from 0.625:1 to 10:1.

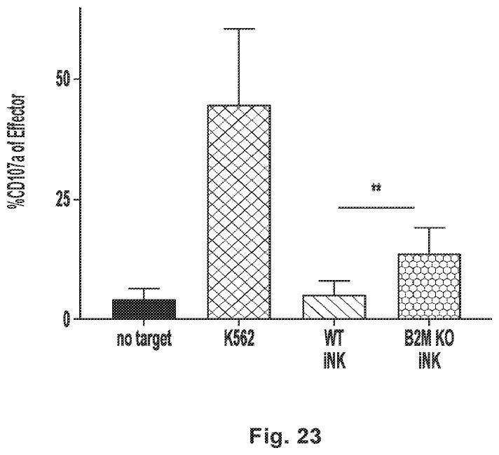

As described above, given that differentiation from iPSCs to iNKs can be laborious and time consuming, a proxy cell line (K562) was utilized for primary transgenic construct screening purposes. K562 is a known, commercially-available, immortalized myelogenous leukemia cell line, it has relatively low MHC-1 expression levels (similar to B2M KO iNKs), is easily transformed or transduced, and is suitable for generating a robust immune cell degranulation response. As shown in FIG. 23, the proportion of activated HDNKs expressing CD107a as a marker of degranulation was above 40% when HDNKs were co-cultured overnight (16 h) with K562 cells, compared to below 10% for HDNKs cultured alone. In addition, when HDNKs were co-cultured overnight with WT iNK cells, a similarly low (below 10%) CD107a rate was observed, significantly lower than the rate observed when HDNKs were co-cultured overnight with B2M KO iNKs.

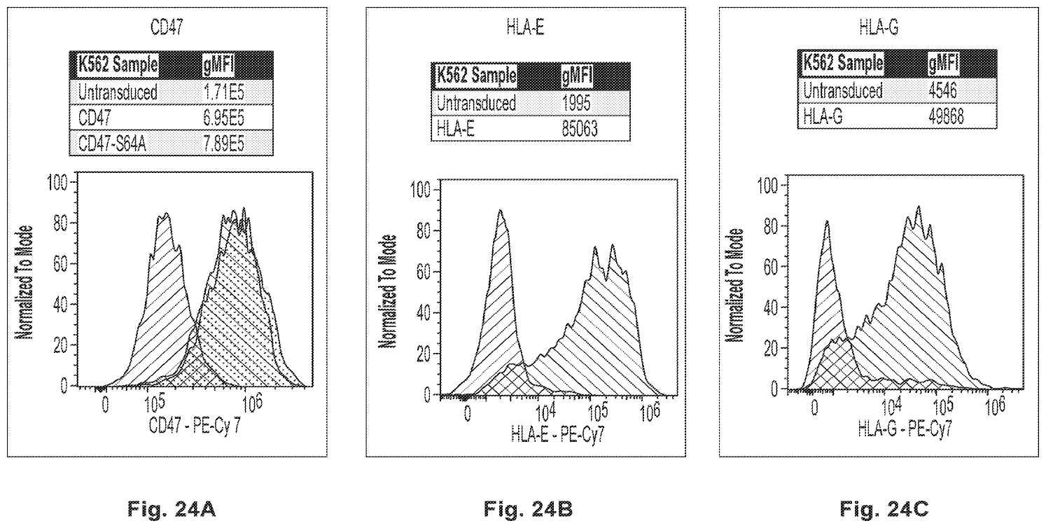

To determine the suitability of Allo shields such as HLA-E, HLA-G, or CD47 for reduction of HDNK cell activation, K562 cells were transformed with Sirion Lentiviral stocks comprising EF1α promoter-driven CD47, HLA-E, or HLA-G constructs (comprising SEQ ID NOs: 1183, 1181, or 1179 respectively). In brief, K562 cells were transduced at an MOI of 10 using spinfection, were then stained 48 hours post-transduction with transgene targeting antibodies, and expression was quantified using flow-cytometry and geometric mean fluorescence intensity (gMFI). As shown in FIG. 24A-24C, K562 cell populations were readily transduced with CD47, HLA-E, or HLA-G.

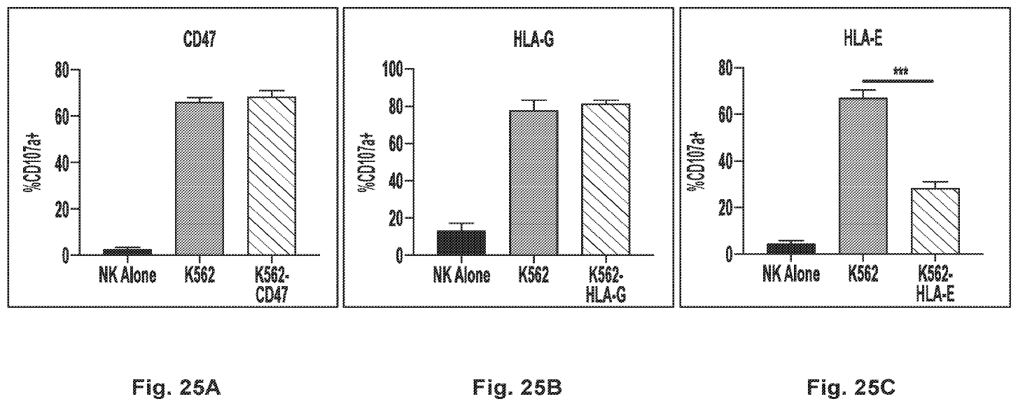

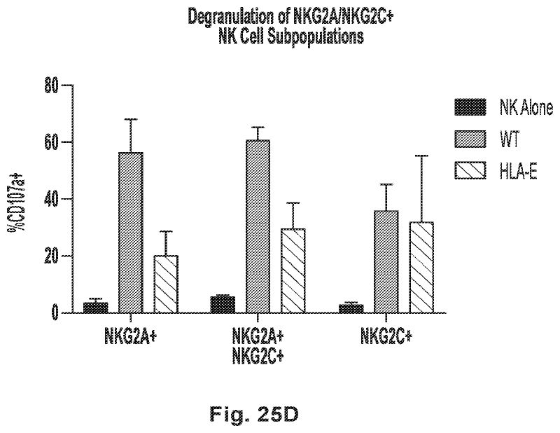

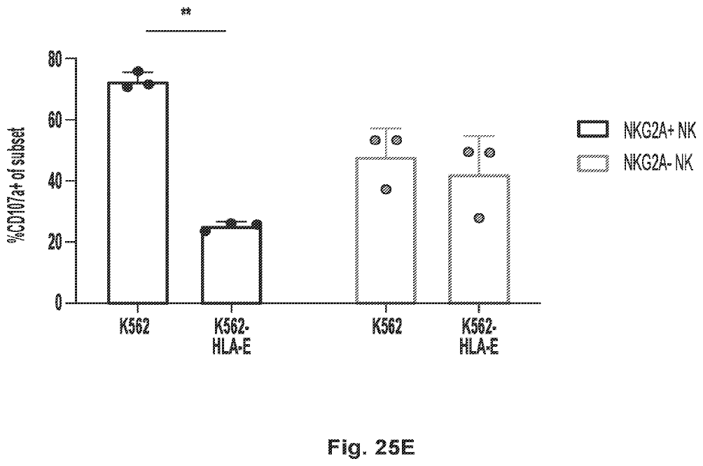

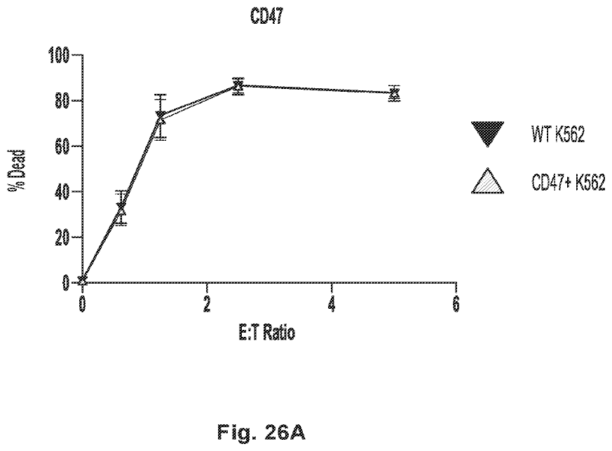

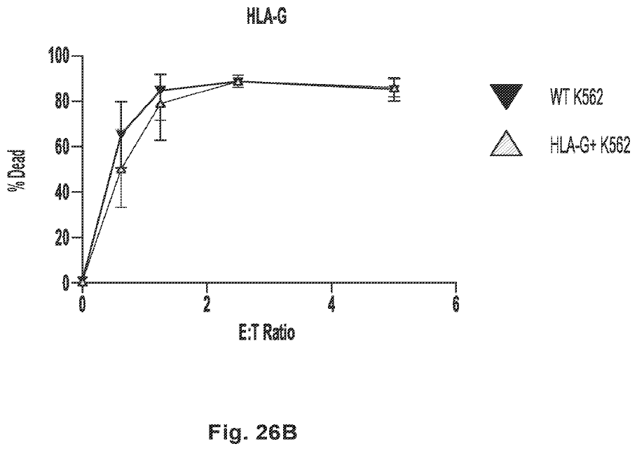

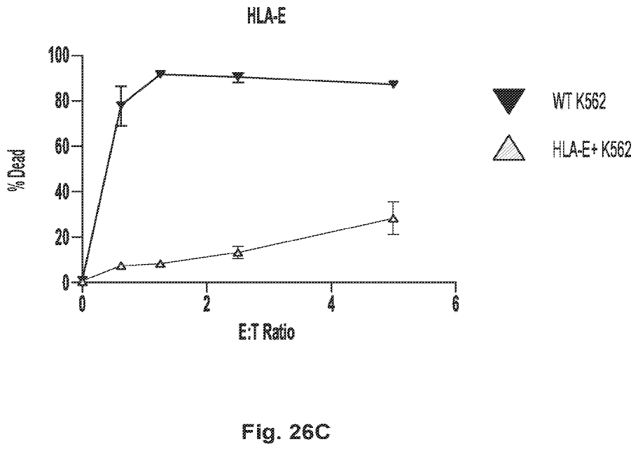

Transduced K562 transgenic cells were then co-cultured with HDNKs, as described above. HDNKs were then analyzed for expression of degranulation marker CD107a in response to overnight 1:1 (E:T) co-culture with vehicle, WT K562 cells, or HLA-E expressing K562 cells (FIG. 25A-25C). Three donor HDNK cell populations were utilized, and a significant reduction (***p<0.001, by ANOVA) in degranulation marker CD107a was observed for K562 cells expressing transgenic HLA-E as compared to WT K562 cells. These data indicate that expression of HLA-E can effectively shield K562 cells from activating HDNKs. Concurrently, the co-cultured HDNK cell populations were sorted by flow-cytometry based upon NKG2A and/or NKG2C marker expression (FIG. 25D). HDNK cell populations labeled NKG2A+ are NKG2C−, HDNK cell populations labeled NKG2C+ are NKG2A−, and HDNK cell populations labeled NKG2A+NKG2C+represent double positive populations for these markers. An additional experiment using NKG2A+ and NKG2A− HDNK cell populations further demonstrated a significant decrease in NKG2A mediated HDNK degranulation upon co-culture with HLA-E expressing K562 cells as opposed to WT K562 cells (FIG. 25E). These data indicate that transgenic HLA-E expression (SEQ ID NO: 1181) in K562 cells can effectively inhibit NKG2A+mediated HDNK degranulation; additional analogous experiments were conducted using freshly thawed HDNKs derived from two different donors, similar results were obtained (data not shown). Finally, transgenic K562 cells were also co-cultured with HDNK cells at various E:T Ratios (0 to 6), and cell death was measured; as shown in FIG. 26A-26C, transgenic expression of HLA-E effectively shielded K562 from HDNK induced cell death.

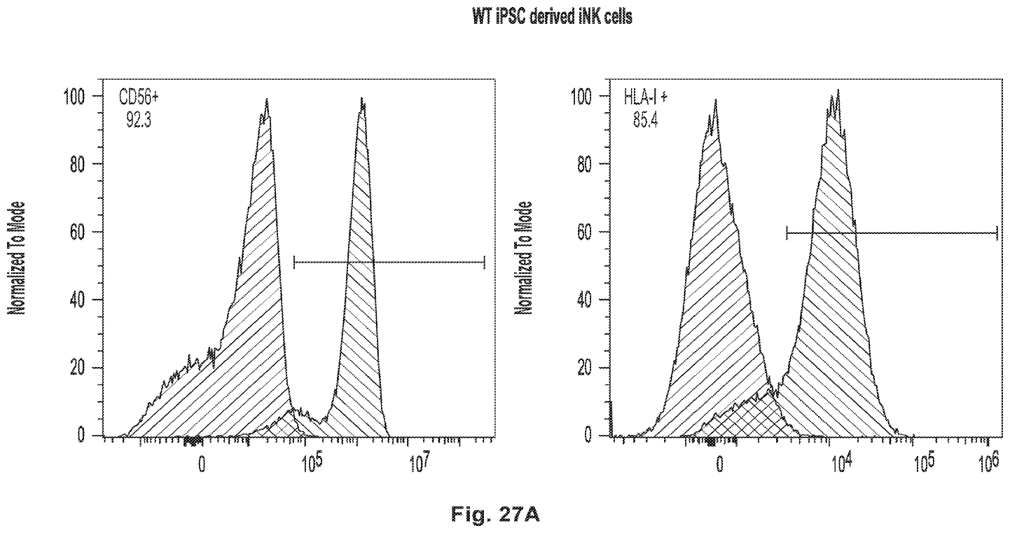

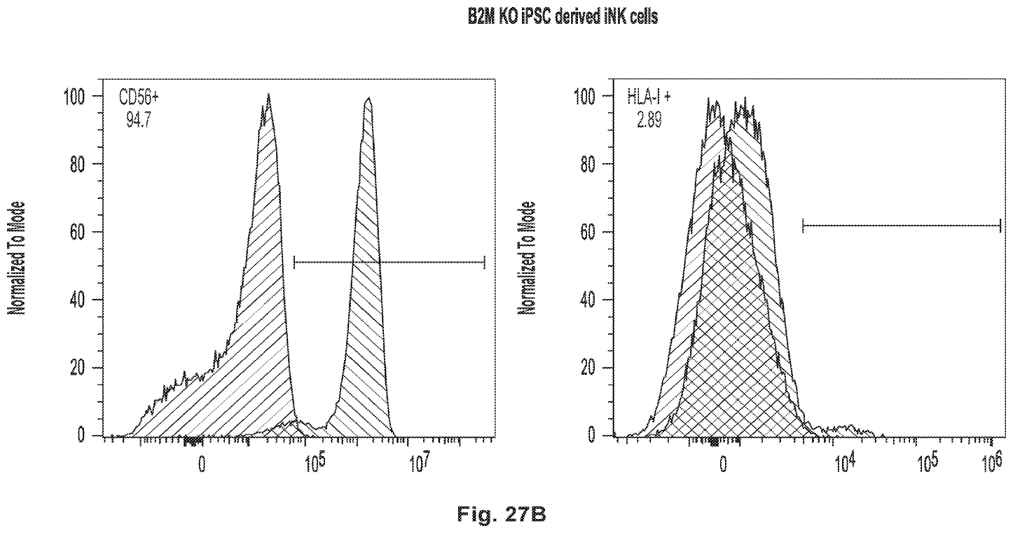

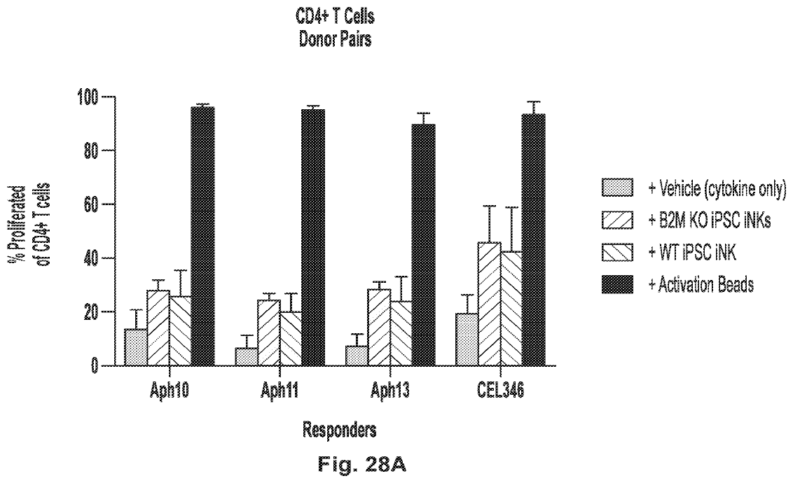

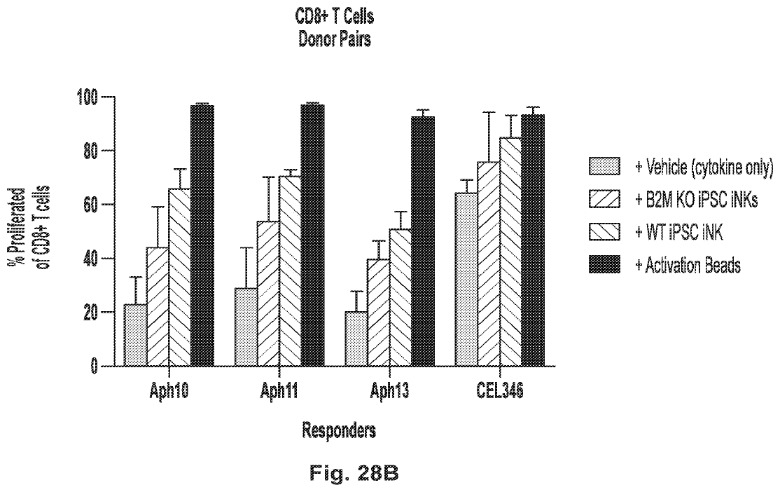

Next, B2M KO iPSC clonal lines were further characterized following differentiation into iNK cells. FIGS. 27A and 27B depict CD56 and/or MHC class 1 (HLA-1) surface expression in WT iPSCs (FIG. 27A) or B2M KO iPSCs (FIG. 27B) at day 47 of differentiation. These results confirmed that CD56 was expressed in the majority of cells (>90% for both cell types), while HLA-1 expression was ˜85% in WT iPSC derived cells, but negligible (˜3%) in B2M KO iPSC derived cells. The day after confirmation of CD56/HLA-1 expression, iNK cells were co-cultured overnight with mixed PBMCs in X-vivo15 Media with 5% AB serum and cytokines (100iU/IL-2 and 20 ng/IL-15). FIG. 28A depicts the percentages of CD4+ T cells that proliferated following Mixed Lymphocyte Reaction (MLR) experiments comprising PBMC responders Aph10, Aph11, Aph13, or CEL346 that were co-cultured overnight at a 2:1 (E:T) ratio (100K PBMC to 50K iNK) with the noted stimulators (vehicle (cytokine only), B2M KO iNKs, WT iNKs, or activation beads). The results in FIG. 28A were collated from two independent experiments (day 44 and day 48 of differentiation from iPSC to iNK). FIG. 28B depicts the percentages of CD8+ T cells that proliferated following the aforementioned experiment. On average, the percentage of CD8+ T cells proliferating in response to B2M KO iNKs was lower than for WT iNKs.

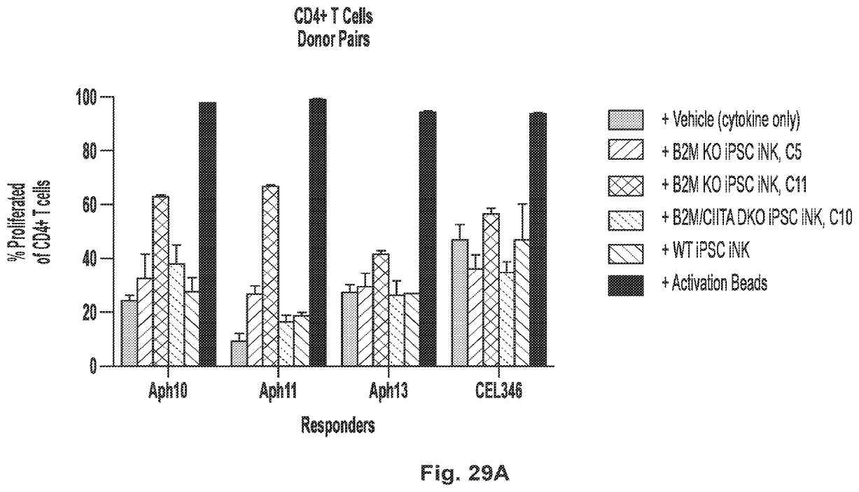

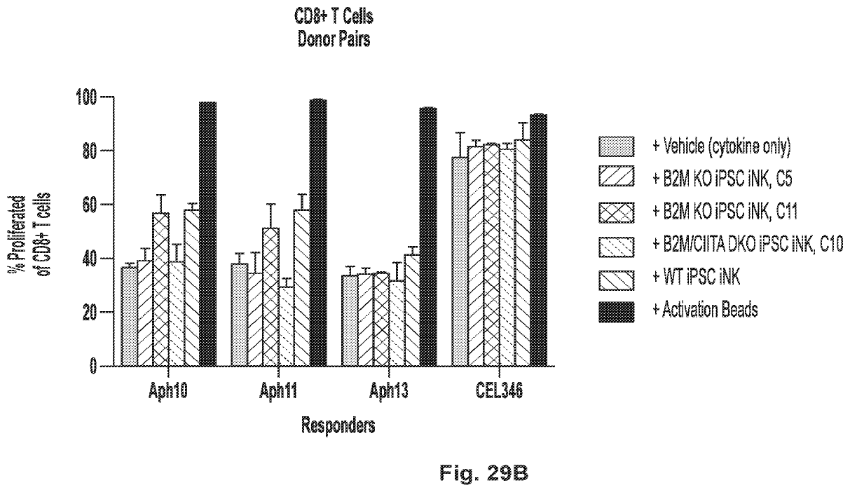

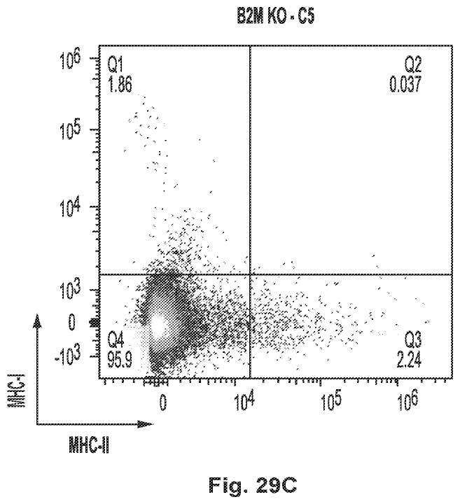

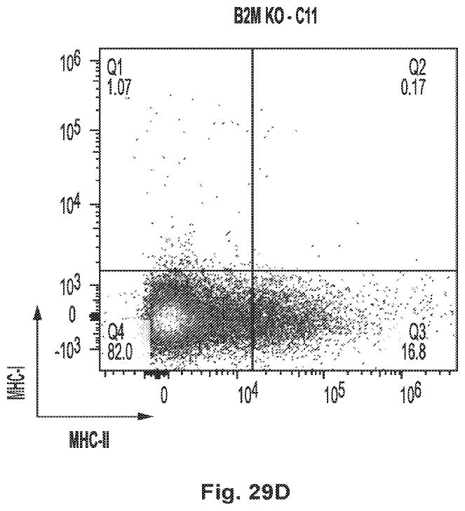

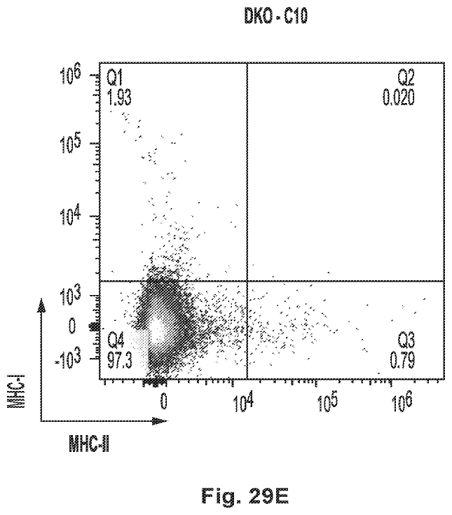

It is known that B2M is required for MHC-1 expression, while CIITA (Class II Major Histocompatibility Complex Transactivator) is required for MHC-II expression. Thus, knocking out CIITA may reduce CD4+ T cell alloresponse. B2M/CIITA double KO iPSC cell lines were created using RNPs comprising AsCpf1 (SEQ ID NO: 1148), a guide RNA targeting B2M (with a targeting domain sequence of AGTGGGGGTGAATTCAGTGTA (as presented as DNA); SEQ ID NO: 412), and a guide RNA targeting CIITA. As shown in FIG. 29A, CD4+ T cells proliferated following MLR experiments performed as described above, but the data showed enhanced CD4+ T cell alloresponse to MHC-II++ iNKs. In addition, CD8+ T cells exhibited a lower level of proliferation in response to B2M KO or DKO iNKs when compared to WT iNKs (FIG. 29B). Of note, MHC-II expression levels in B2M KO clone 5 (FIG. 29C) were more similar to MHC-II expression levels in B2M/CIITA DKO clone 10 (FIG. 29E) than to B2M KO clone 11 (FIG. 29D).

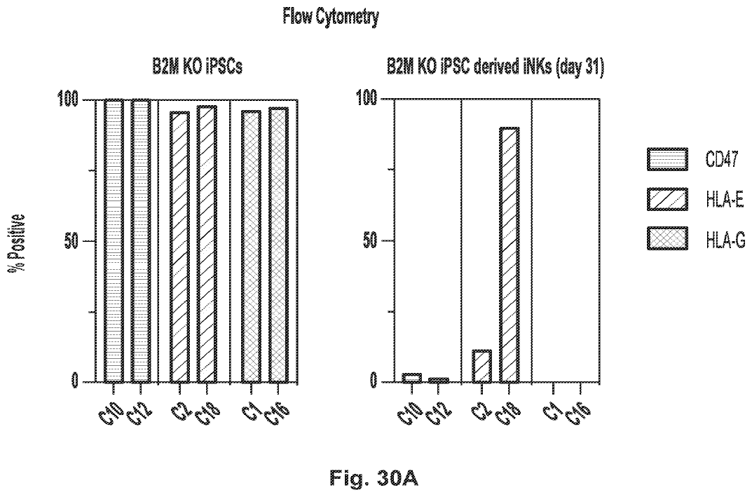

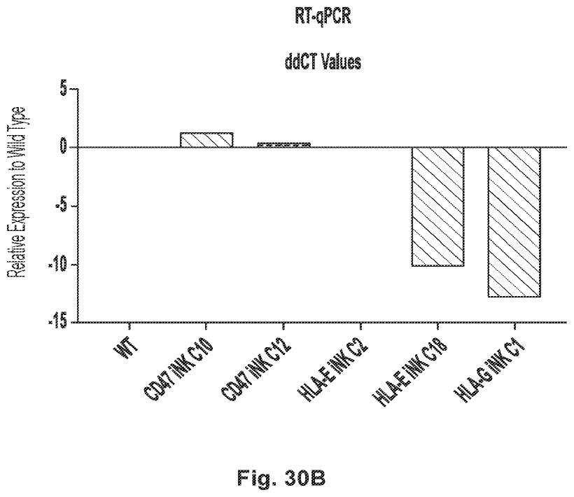

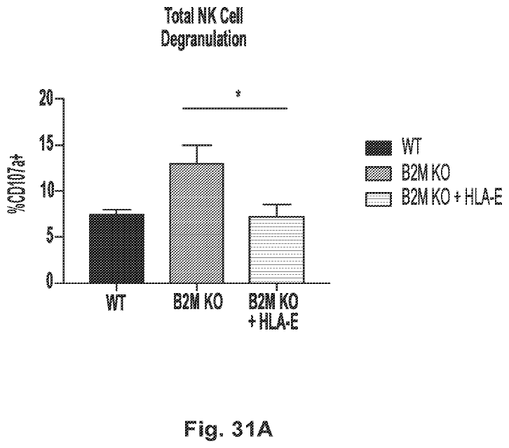

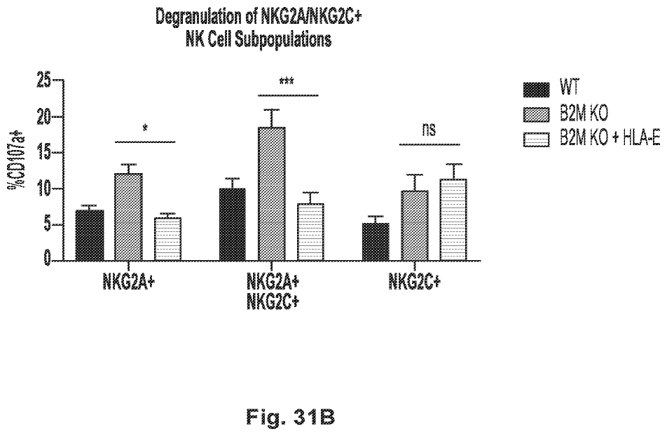

Next, B2M KO iPSCs were transduced with Allo shield constructs HLA-E. HLA-G, or CD47 using lentiviral mediated transduction (comprising SEQ ID NOs: 1181, 1179, or 1183 respectively). Flow cytometry was utilized to confirm successful transgene expression in B2M KO iPSCs (shown in FIG. 30A, left panel). Clonal lines were then differentiated to iNKs, and transgene expression by iNK cells at day 31 was assessed with flow cytometry. As shown in FIG. 30A (right panel), B2M KO/HLA-E+iPSC C18 derived iNKs expressed sufficiently high levels of a transgenic protein. These findings were confirmed using qRT-PCR on a subset of the population (FIG. 30B). HDNK expression of degranulation marker CD107a was assessed following overnight 1:1 (E:T) co-culture with WT iPSC derived iNKs (WT), B2M KO iPSC derived iNKs (B2M KO), or B2M KO iPSC derived iNKs expressing transgenic HLA-E (B2M KO+HLA-E). As shown in FIG. 31A, HLA-E protected B2M KO iNKs from HDNK cytotoxicity (representative data collated from 5 donors; error bars represent SEM; *P<0.05 by ANOVA). In addition, as shown in FIG. 31B, sorting HDNK cell subpopulations by NKG2A and/or NKG2C status demonstrated that HLA-E expression in B2M KO iNK cells effectively inhibited NKG2A+mediated HDNK degranulation (representative data collated from 5 donors; error bars represent SEM; *P<0.05. ***P<0.001 by ANOVA). These results showed that HLA-E functioned as an effective Allo shield, protecting B2M KO iPSC derived iNKs from NKG2A+mediated HDNK cell degranulation (as measured by CD107a expression).

### Example 8: Generation of B2M Knockout and/or HLA-E Knock-In T Cells

The present example describes gene editing of populations of T cells using viral vector transduction. Following editing, cells were subjected to various assays such as flow cytometry, ddPCR, next-generation sequencing, or functional tumor killing assays to determine KO/KI efficiency and/or efficacy.

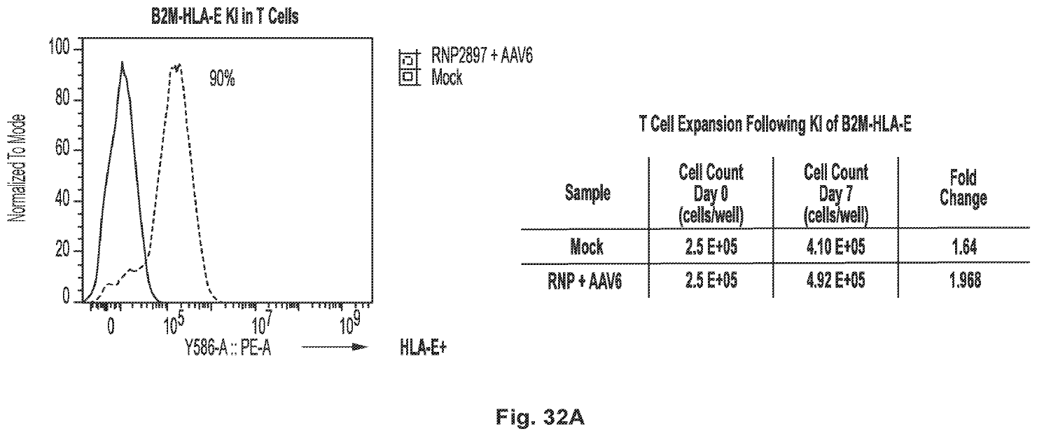

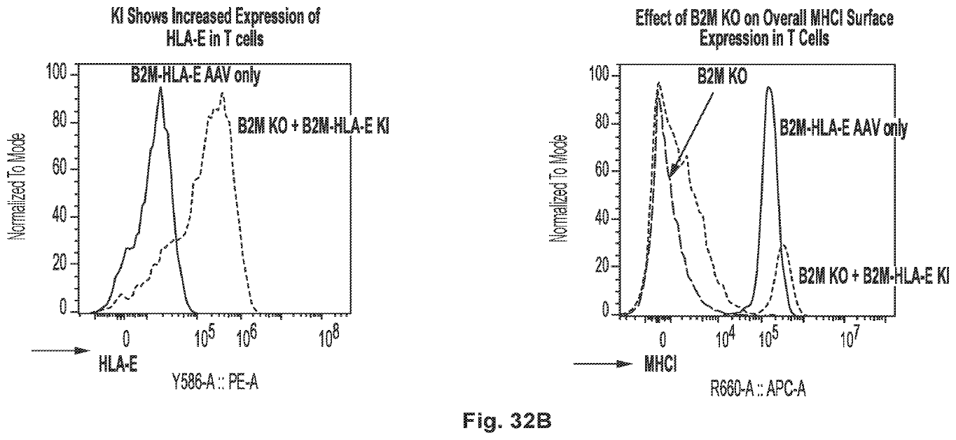

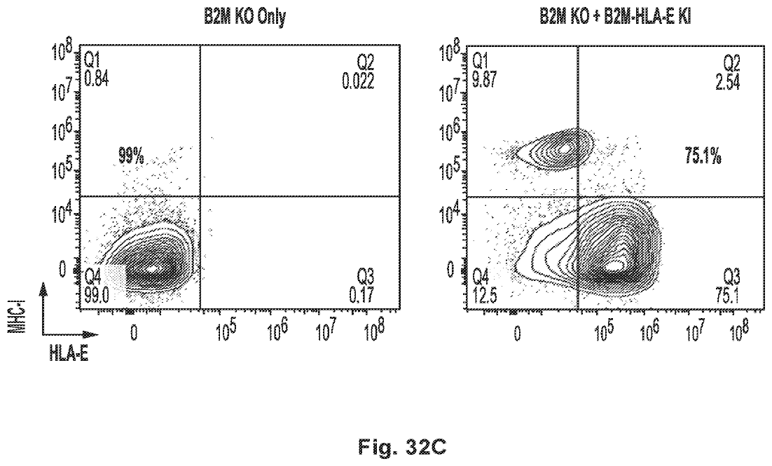

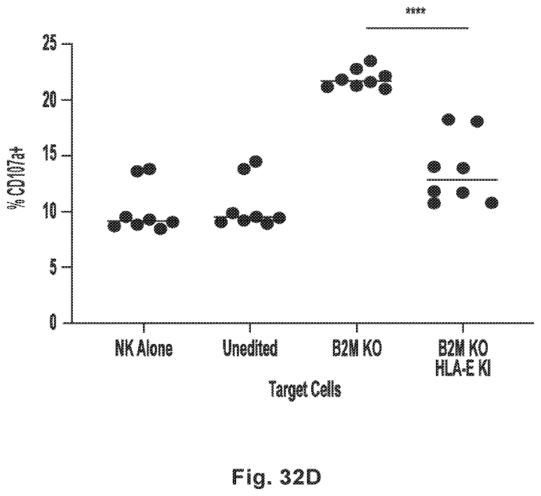

T cells were thawed in a bead bath as known in the art and were removed from the bath on day two. Cells were electroporated on day four after thawing. Briefly, 250,000 T cells per well in a Lonza 96-well cuvette were suspended in buffer P2 and electroporated with RNP comprising gRNA RSQ22337 (SEQ ID NO: 1178) and Cas12a (SEQ ID NO: 1148) targeting the GAPDH gene (1 μM RNP) or with media control, using various pulse codes. Appropriate media was added to cells immediately after electroporation and cells were allowed to recover for 15 minutes. AAV6 viral particles comprising a donor plasmid construct containing a knock-in cassette with a cargo of B2M-HLA-E, or vector control were then added to T cells at varying multiplicity of infection (MOI) concentrations (1E4, 1E5, or 1E6 MOI (vg/cell)). The donor plasmids were designed as described in Example 6, with a 5′ codon-optimized coding portion of GAPDH exon 9 optimized to prevent further binding of the gRNA targeting domain sequence of the guide RNA (RSQ22337)), an in-frame sequence encoding the P2A self-cleaving peptide (“P2A”), an in-frame coding sequence for a cargo sequence (e.g., B2M-HLA-E) (“Cargo”), a stop codon and polyA signal sequence. T cells were split two days later, and then every 48 hours until they were analyzed by flow cytometry or otherwise utilized. T cells were sorted using flow cytometry seven days post electroporation to determine successful transduction, transformation, editing, knock-in cassette integration, and/or expression events. B2M-HLA-E KI cells expressed a higher level of HLA-E when compared to control cells and were viable (see FIG. 32A).

As shown in FIG. 32B, HLA-E and/or MHC1 surface expression in T cells was modified using methods as described herein. The left panel of FIG. 32B depicts HLA-E surface expression in T cells transduced with AAV6 comprising a B2M-HLA-E cargo targeted for knock-in at GAPDH at 5E4 MOI and transformed with a B2M targeting RNP and with 1 μM of RNPs comprising Cas12a (SEQ ID NO: 1148) with RSQ22337 (SEQ ID NO: 1178), compared to mock transduced control cells exposed to AAV6 only, without RNPs. The right panel of FIG. 32B depicts MHC1 surface expression in T cells transduced with AAV6 comprising a B2M-HLA-E cargo targeted for knock-in at GAPDH at 5E4 MOI and transformed with a B2M targeting RNP and with 1 μM of RNPs comprising Cas12a (SEQ ID NO: 1148) with RSQ22337 (SEQ ID NO: 1178), compared to mock transduced control cells exposed to AAV6 only without RNPs, or B2M KO control T cells. Representative flow cytometry plots for B2M KO control T cells and B2M KO/B2M-HLA-E KI T cells—and corresponding to the right panel of FIG. 32B—are shown in FIG. 32C.

B2M KO/B2M-HLA-E KI T cells as described above were tested in a degranulation assay as described herein. In brief, healthy donor NK (HDNK) cells from four donors were cultured alone (NK Alone) overnight or co-cultured overnight at a 1:1 E:T ratio with unedited T cells (Unedited), B2M KO control T cells (B2M KO), or B2M KO/B2M-HLA-E KI T cells (B2M KO HLA-E KI). Following the overnight culturing, cells were analyzed by flow cytometry. As seen in FIG. 32D, a significantly smaller percentage of CD107a+ cells were observed when HDNKs were co-cultured with B2M KO/B2M-HLA-E KI T cells as compared to with B2M KO control T cells. These data indicate that transgenic HLA-E expression in B2M KO T cells can effectively inhibit HDNK degranulation and avoid an NK cell response.

### Example 9: Generation of CD19 CAR/HLA-E DKI in T Cells

The present example describes gene editing of populations of T cells. Following editing, cells were subjected to various assays such as flow cytometry.

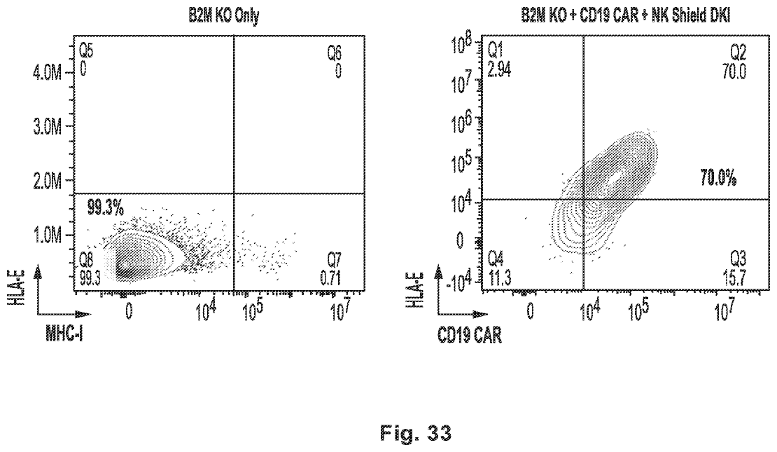

T cells isolated from peripheral blood mononuclear cells and frozen in cryopreservation media were thawed in a bead bath as known in the art. A CD19 CAR and B2M-HLA-E bicistronic cargo was knocked-in using methods disclosed herein using a donor template comprising the cargo of interest, RNP comprising gRNA RSQ22337 (SEQ ID NO: 1178) and Cas12a (SEQ ID NO: 1148) targeting the GAPDH gene (1 μM RNP), and a B2M-targeting RNP. The donor templates were designed as described in Example 6, with a 5′ codon-optimized coding portion of GAPDH exon 9 optimized to prevent further binding of the gRNA targeting domain sequence of the guide RNA (RSQ22337 (SEQ ID NO: 1178)), an in-frame sequence encoding the P2A self-cleaving peptide (“P2A”), an in-frame coding sequence for a cargo sequence (e.g., CD19 CAR (e.g., SEQ ID NO: 1232) and B2M-HLA-E (e.g., SEQ ID NO: 1230), separated by a P2A linker sequence) (“Cargo”), a stop codon and polyA signal sequence. T cells were sorted using flow cytometry to determine successful transformation, editing, knock-in cassette integration, and/or expression events. As seen in FIG. 33, the B2M KO/CD19 CAR/B2M-HLA-E (NK Shield) DKI T cells were approximately 99.3% negative for B2M (MHC1) expression and approximately 70% positive for simultaneous expression of HLA-E and CD19 CAR. These data demonstrates that modified T cells produced by methods disclosed herein can efficiently express both CD19 CAR and B2M-HLA-E.

### Example 10: Generation of CD19 CAR KI in Combination with TRAC, B2M, and CIITA KO in T Cells

The present example describes gene editing of populations of T cells. Following editing, cells were subjected to various assays such as flow cytometry, next generation sequencing (NGS), and/or an in vitro tumor killing assay.

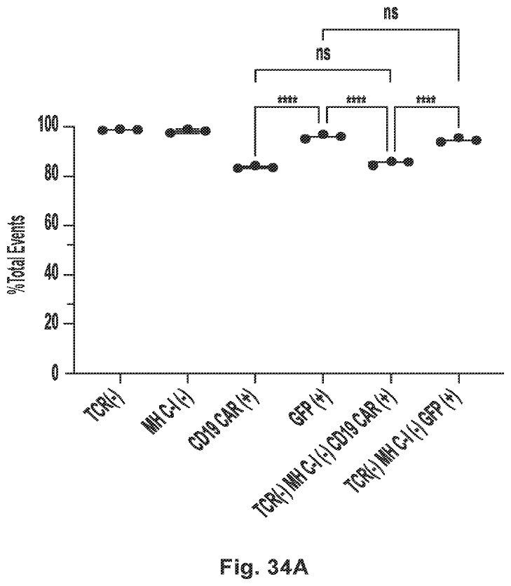

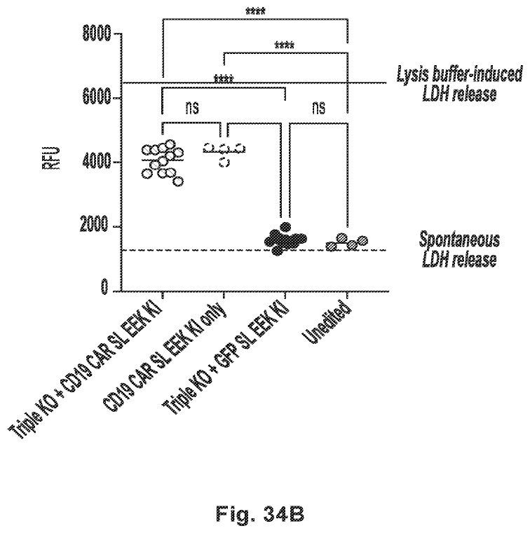

Highly defined engineered T cells comprising multiple edits can be generated using a one-step electroporation and transformation process in which three Cas12a (SEQ ID NO: 1148) RNPs targeting three loci (TRAC, B2M and GAPDH) and a donor template comprising a CD19 CAR or GFP cargo for knock-in at the GAPDH locus are applied to the T cells. The GAPDH-targeted RNP comprised gRNA RSQ22337 (SEQ ID NO: 1178). As shown in FIG. 34A, the one-step process generated about the same percentage of cells comprising CD19 CAR or GFP knock-ins as performing the CD19 CAR or GFP knock-in alone (e.g., without the TRAC (TCR) and B2M (MHC-I) knock-outs) as measured by flow cytometry and NGS.

In addition, T cells were edited to generate multiple knock-outs (KO) at the TRAC, B2M, and CIITA loci as well as a CD19 CAR or GFP cargo knock-in (KI) at the GAPDH locus using a one-step process wherein four Cas12a (SEQ ID NO: 1148) RNPs (specific to TRAC, B2M, CIITA, and GAPDH) and a donor template comprising a CD19 CAR or GFP cargo designed to integrate within the GAPDH locus were applied to the cells at once. The GAPDH-targeted RNP comprised gRNA RSQ22337 (SEQ ID NO: 1178). T cells comprising the triple (TRAC, B2M, and CIITA) KO in combination with the CD19 CAR or GFP KI were examined using an in vitro tumor killing assay. In brief, T cells were co-cultured with Nalm6 cells for 24 hours at an E:T of 1. Following co-culture, BATDA release (as relative fluorescence units (RFUs)) was assessed using a time-resolved fluorometer. T cells comprising the CD19 CAR KI (with or without the triple KO) displayed significantly greater cytotoxicity, as measured by BATDA release, than unedited T cells or T cells comprising the GFP KI with the triple KO (FIG. 34B). These results demonstrate that the cells described herein are suitable for targeting tumors and/or cancerous cells.

### Example 11: Generation and Characterization of B2M Knockout and/or HLA-E Knock-in iPSCs and iNKs

To protect allogeneic iNKs from recipient immune system rejection, HLA class I expression was eliminated by knocking out beta-2 microglobulin (B2M), using methods as described herein. In brief, iPSCs were created as described in Example 1; these cells were then transformed with an RNP complex comprising Cas12a (SEQ ID NO: 1148) and a gRNA targeting B2M (SEQ ID NO: 412). Additionally, a cargo was knocked-in using methods disclosed herein using a donor template comprising the cargo of interest and a RNP comprising gRNA RSQ22337 (SEQ ID NO: 1178) and Cas12a (SEQ ID NO: 1148) targeting the GAPDH gene. The cargo of interest comprised an HLA-E construct (encoding SEQ ID NO: 1182 or SEQ ID NO: 1243) comprising (i) an HLA-G signal peptide comprising VMAPRTLIL (SEQ ID NO: 1236) or VMAPRTLVL (SEQ ID NO: 1238), (ii) a B2M polypeptide, and (iii) HLA-E. Cells were allowed to recover and were expanded as described in Example 1. Successful transgene expression was confirmed and clonal lines were then differentiated to iNKs.

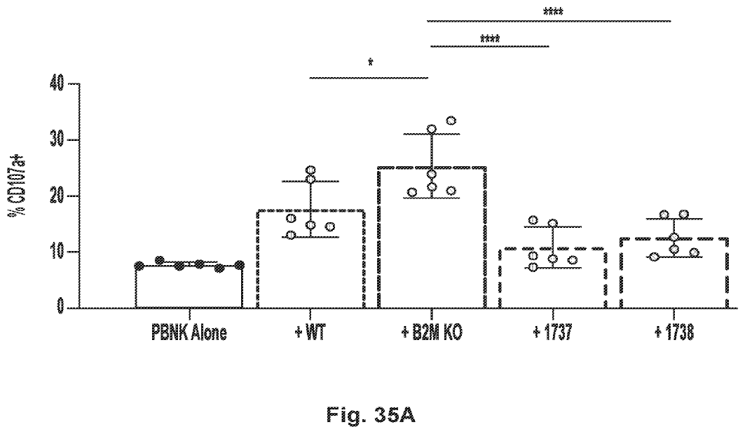

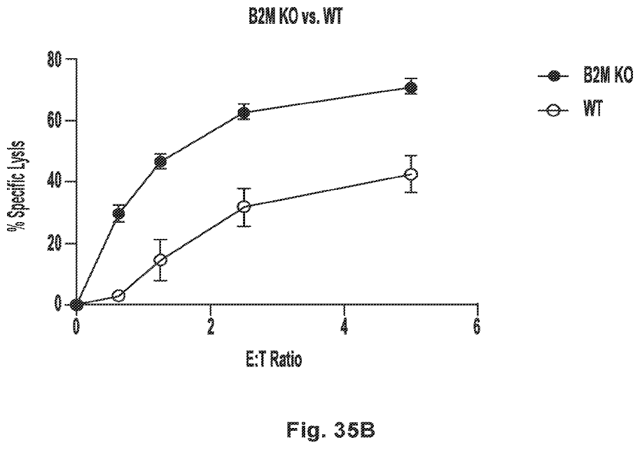

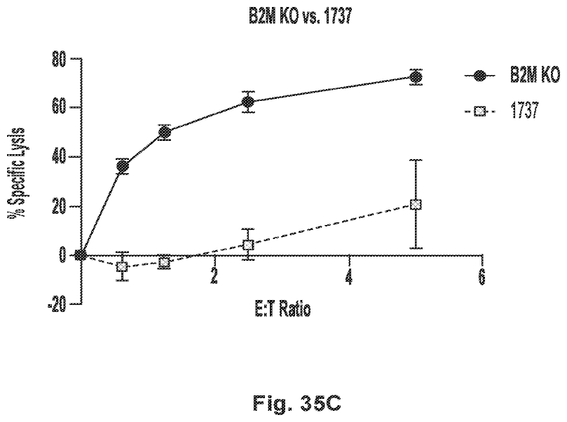

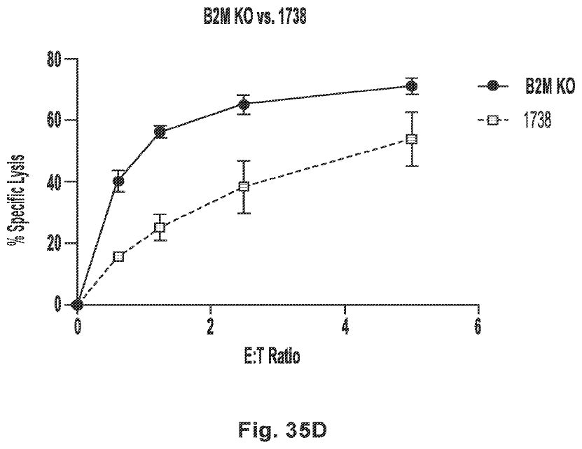

Generated B2M KO iNK cells and B2M KO/HLA-E KI INK cells were evaluated for the ability to induce degranulation of peripheral blood NK (PBNK) cells. PBNK cell expression of degranulation marker CD107a was assessed following overnight co-culture at an E:T ratio of 1:1 with WT iNK cells (WT), B2M KO iNK cells (B2M KO), or B2M KO iNK cells expressing transgenic HLA-E comprising a fused HLA-G signal peptide sequence comprising VMAPRTLIL (SEQ ID NO: 1236) (+1737) or VMAPRTLVL (SEQ ID NO: 1238) (+1738). Cells were co-cultured in the presence of anti-CD107a antibody and monensin. Cells were then stained with a viability dye and antibodies to detect CD56 and HLA-E, and fixed and run on a Quanteon flow cytometer. As shown in FIG. 35A, the level of PBNK cell degranulation (as measured by the percentage of CD107a+PBNK cells) induced by B2M KO iNK cells was significantly increased as compared to WT iNK cells. Meanwhile, the level of PBNK cell degranulation induced by B2M KO/HLA-E KI INK cells was significantly decreased as compared to B2M KO iNK cells and comparable or lower than seen with WT iNK cells. These results demonstrate that transgenic expression of HLA-E can effectively shield B2M KO iNK cells from activating PBNKs, and thus decrease PBNK cell degranulation.

Further, the lysis of iNK cells was evaluated following overnight co-culture across various E:T ratios (from 0 to 5). PBNKs were co-cultured with a 1:1 mixture of two target cell populations that were each dyed with a cell trace dye: CFSE or CTV. PBNKs were plated at increasing E:T ratios (0.625:1-5:1) to the mixed target cell population. After overnight incubation, cells were stained with a viability dye, then fixed and run on a Quanteon flow cytometer. B2M KO iNK cells displayed a greater susceptibility to PBNK cell cytotoxicity than WT iNK cells as shown in FIG. 35B. On the other hand, B2M KO/HLA-E KI INK cells showed lessened susceptibility to PBNK cell cytotoxicity than B2M KO iNK cells (FIG. 35C-D). This decrease in lysis was observed with expression of HLA-E comprising a fused HLA-G signal peptide sequence comprising either VMAPRTLIL (SEQ ID NO: 1236) (1737) (FIG. 35C) or VMAPRTLVL (SEQ ID NO: 1238) (1738) (FIG. 35D). These results display that HLA-E functioned to effectively protect B2M KO iNK cells from PBNK cell cytotoxicity.

### EQUIVALENTS

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

We claim:

1. A pluripotent stem cell, wherein the stem cell comprises:
(i) a genomic edit that results in loss of function of Beta-2-Microglobulin (B2M), and
(ii) a genome comprising an exogenous nucleic acid comprising a nucleotide sequence encoding an HLA-E polypeptide.

2. The pluripotent stem cell of claim 1, wherein the exogenous nucleic acid comprises a nucleotide sequence encoding a portion of a B2M polypeptide.

3. The pluripotent stem cell of claim 1 or 2, wherein the exogenous nucleic acid comprises a nucleotide sequence encoding an HLA-G signal peptide.

4. The pluripotent stem cell of claim 3, wherein the HLA-G signal peptide comprises an amino acid sequence of VMAPRTLFL (SEQ ID NO: 1235), VMAPRTLIL (SEQ ID NO: 1236), VMAPRTVLL (SEQ ID NO: 1237), and/or VMAPRTLVL (SEQ ID NO: 1238).

5. The pluripotent stem cell of claim 3 or 4, wherein the exogenous nucleic acid comprises, from 5′ to 3′, the nucleotide sequence encoding the HLA-G signal peptide, the nucleotide sequence encoding the portion of the B2M polypeptide, and the nucleotide sequence encoding the HLA-E polypeptide.

6. The pluripotent stem cell of any one of claims 3-5, wherein the exogenous nucleic acid comprises a first linker sequence between the nucleotide sequence encoding the HLA-G signal peptide and the nucleotide sequence encoding the portion of the B2M polypeptide, and a second linker sequence between the nucleotide sequence encoding the portion of the B2M polypeptide and the nucleotide sequence encoding the HLA-E polypeptide.

7. The pluripotent stem cell of any one of claims 1-6, wherein the exogenous nucleic acid consists of or comprises the nucleotide sequence of SEQ ID NO: 1181 or 1230.

8. The pluripotent stem cell of any one of claims 1-7, wherein the exogenous nucleic acid encodes a polypeptide that consists of or comprises the amino acid sequence of SEQ ID NO: 1182, 1231, 1243, 1244, 1245, or 1246.

9. The pluripotent stem cell of any one of claims 1-8, wherein the pluripotent stem cell comprises a genomic edit that results in a loss of function of an agonist of the TGF beta signaling pathway, a genomic edit that results in loss of function of Cytokine Inducible SH2 Containing Protein (CISH), a genomic edit that results in loss of function of class II, major histocompatibility complex, transactivator (CIITA), and/or a genomic edit that results in a loss of function of adenosine A2a receptor (ADORA2A).

10. The pluripotent stem cell of any one of claims 1-9, wherein the exogenous nucleic acid is in frame with and downstream (3′) of an exogenous coding sequence or partial coding sequence of an essential gene.

11. The pluripotent stem cell of claim 10, wherein the essential gene is a housekeeping gene, e.g., a gene listed in Table 13.

12. The pluripotent stem cell of claim 11, wherein the essential gene encodes glyceraldehyde 3-phosphate dehydrogenase (GAPDH).

13. The pluripotent stem cell of any of claims 10 to 12, wherein the pluripotent stem cell is produced by a method comprising contacting a pluripotent stem cell with;
(i) a nuclease that causes a break within the endogenous coding sequence of the essential gene, and
(ii) a donor template that comprises a knock-in cassette comprising the exogenous nucleic acid in frame with and downstream (3′) of an exogenous coding sequence or partial coding sequence of the essential gene, wherein the knock-in cassette is integrated into the genome of the cell by homology-directed repair (HDR) of the break.

14. The pluripotent stem of cell of any one of claims 1-13, wherein the pluripotent stem cell is an induced pluripotent stem cell (iPSC).

15. A differentiated cell, wherein the differentiated cell is a daughter cell of the pluripotent stem cell of any one of claims 1-14.

16. The differentiated cell of claim 15, wherein the differentiated cell is an immune cell.

17. The differentiated cell of claim 16, wherein the differentiated cell is a lymphocyte.

18. The differentiated cell of claim 17, wherein the differentiated cell is an induced natural killer (iNK) cell.

19. The differentiated cell of any one of claims 15-18, for use as a medicament.

20. The differentiated cell of any one of claims 15-19, for use in the treatment of a disease, disorder, or condition, e.g., a tumor and/or a cancer.

21. A progeny or daughter cell of the differentiated cell of any one of claims 15-20.

22. A population of cells comprising the pluripotent stem cell, the differentiated cell, or the progeny or daughter cell of any one of claims 1-21.

23. The population of cells of claim 22, wherein the population of cells comprises the iNK cell of claim 18.

24. The population of cells of claim 23, characterized in that, when contacted with natural killer (NK) cells, a level of activation of NK cells is decreased relative to a reference level of activation of NK cells when contacted with a reference population of cells.

25. The population of cells of claim 23, characterized in that, when contacted with NK cells, a level of degranulation of NK cells is decreased relative to a reference level of degranulation of NK cells when contacted with a reference population of cells.

26. The population of cells of claim 23, characterized in that, when contacted with NK cells, a level of cell death and/or lysis of the population of cells is decreased relative to a reference level of cell death and/or lysis of a reference population of cells when contacted with NK cells.

27. The population of cells of any one of claims 24-26, wherein the NK cells are human donor NK cells and/or peripheral blood NK cells.

28. The population of cells of any one of claims 24-27, wherein the reference population of cells does not comprise iNK cells comprising a genome comprising the exogenous nucleic acid.

29. The population of any one of claims 24-28, wherein the reference population of cells does not comprise iNK cells comprising the genomic edit that results in loss of function of B2M.

30. A pharmaceutical composition comprising the pluripotent stem cell, the differentiated cell, the progeny or daughter cell, or the population of cells of any one of claims 1-29.

31. The pharmaceutical composition of claim 30, comprising a pharmaceutically acceptable carrier.

32. A method of treating a condition, disorder, and/or disease, comprising administering to a subject suffering therefrom the pluripotent stem cell, the differentiated cell, the progeny or daughter cell, or the population of cells of any one of claims 1-29.

33. The method of claim 32, wherein the subject is suffering from a tumor, e.g., a solid tumor.

34. The method of claim 32, wherein the subject is suffering from a cancer.

35. A method, comprising administering to a subject the pluripotent stem cell, the differentiated cell, the progeny or daughter cell, or population of cells of any one of claims 1-29.

36. The method of claim 35, wherein the subject is suffering from a tumor, e.g., a solid tumor.

37. The method of claim 35, wherein the subject is suffering from a cancer.

38. A method, comprising administering to a subject the pharmaceutical composition of claim 30 or 31.

39. The method of any one of claims 32-38, wherein the pluripotent stem cell, the differentiated cell, the progeny or daughter cell, or the population of cells is allogeneic to the subject.

40. The method of any one of claims 32-39, wherein the subject is a human.

41. A method of manufacturing a cell, the method comprising:
(a) knocking-out a gene of the cell, wherein the gene encodes Beta-2-Microglobulin (B2M); and
(b) knocking-in to the genome of the cell an exogenous nucleic acid comprising a nucleotide sequence encoding an HLA-E polypeptide, wherein the exogenous nucleic acid is knocked-in in frame and downstream (3′) of an essential gene.

42. The method of claim 41, wherein knocking-out comprises contacting the cell with an RNP complex comprising:
(i) an RNA-guided nuclease, and
(ii) a guide RNA comprising a targeting domain sequence comprising a nucleotide sequence selected from the group consisting of SEQ ID NO: 365-576.

43. The method of claim 42, wherein the RNA-guided nuclease is a CRISPR/Cas nuclease.

44. The method of any one of claims 41-43, wherein knocking-in comprises contacting the cell with:
(i) a nuclease that causes a break within an endogenous coding sequence of the essential gene, and
(ii) a donor template that comprises a knock-in cassette comprising the exogenous nucleic acid in frame with and downstream (3′) of an exogenous coding sequence or partial coding sequence of the essential gene, wherein the knock-in cassette is integrated into the genome of the cell by homology-directed repair (HDR) of the break.

45. The method of claim 44, wherein the nuclease is a CRISPR/Cas nuclease, and knocking-in further comprises contacting the cell with a guide molecule for the CRISPR/Cas nuclease.

46. The method of any one of claims 41-45, wherein the cell is a pluripotent stem cell, optionally an induced pluripotent stem cell (iPSC).

47. The method of any one of claims 41-45, wherein the cell is a differentiated cell.

48. The method of any one of claims 41-45, wherein the cell is an induced NK (INK) cell.

49. The method of any one of claims 41-48, wherein the essential gene is a housekeeping gene, e.g., a gene listed in Table 13.

50. The method of any one of claims 41-49, wherein the essential gene encodes glyceraldehyde 3-phosphate dehydrogenase (GAPDH).

51. The method of any one of claims 41-50, wherein the method further comprises knocking-out one or more genes of the cell, wherein the one or more genes encode an agonist of the TGF beta signaling pathway, Cytokine Inducible SH2 Containing Protein (CISH), class II, major histocompatibility complex, transactivator (CIITA), and/or adenosine A2a receptor (ADORA2A), or any combination of two or more thereof.

52. A method of reducing a level of killing of a population of cells by NK cells, the method comprising:
(a) knocking-out a gene of cells of the population, wherein the gene encodes Beta-2-Microglobulin (B2M); and
(b) knocking-in to the genome of the cells of the population an exogenous nucleic acid comprising a nucleotide sequence encoding an HLA-E polypeptide, wherein the exogenous nucleic acid is knocked-in in frame and downstream (3′) of an essential gene;
thereby reducing the level of killing of the population of cells when contacted with NK cells relative to a reference level of killing of a reference population of cells when contacted with NK cells.

53. The method of claim 52, wherein the NK cells are human donor NK cells and/or peripheral blood NK cells.

54. The method of claim 52 or 53, wherein the reference population of cells does not comprise cells comprising the exogenous nucleic acid.

55. The method of any one of claims 52-54, wherein the reference population of cells does not comprise cells comprising the genomic edit.

56. The method of any one of claims 52-55, wherein knocking-out comprises contacting the population of cells with an RNP complex comprising:
(i) an RNA-guided nuclease, and
(ii) a guide RNA comprising a targeting domain sequence comprising a nucleotide sequence selected from the group consisting of SEQ ID NO: 365-576.

57. The method of claim 56, wherein the RNA-guided nuclease is a CRISPR/Cas nuclease.

58. The method of any one of claims 52-57, wherein knocking-in comprises contacting the population of cells with:
(i) a nuclease that causes a break within an endogenous coding sequence of the essential gene, and
(ii) a donor template that comprises a knock-in cassette comprising the exogenous nucleic acid in frame with and downstream (3′) of an exogenous coding sequence or partial coding sequence of the essential gene, wherein the knock-in cassette is integrated into the genome of cells of the population by homology-directed repair (HDR) of the break.

59. The method of claim 58, wherein the nuclease is a CRISPR/Cas nuclease, and knocking-in further comprises contacting the population of cells with a guide molecule for the CRISPR/Cas nuclease.

60. The method of any one of claims 52-59, wherein the population of cells comprises pluripotent stem cells, optionally induced pluripotent stem cells (iPSCs).

61. The method of any one of claims 52-59, wherein the population of cells comprises differentiated cells.

62. The method of any one of claims 52-59, wherein the population of cells comprises induced NK (iNK) cells.

63. The method of any one of claims 52-62, wherein the essential gene is a housekeeping gene, e.g., a gene listed in Table 13.

64. The method of any one of claims 52-63, wherein the essential gene encodes glyceraldehyde 3-phosphate dehydrogenase (GAPDH).

65. The method of any one of claims 52-64, wherein the method further comprises knocking-out one or more genes of cells of the population, wherein the one or more genes encode an agonist of the TGF beta signaling pathway, Cytokine Inducible SH2 Containing Protein (CISH), class II, major histocompatibility complex, transactivator (CIITA), and/or adenosine A2a receptor (ADORA2A), or any combination of two or more thereof.


SEQUENCE LISTING (1252 sequences)

SEQ ID NO: 1 - RNA, 5 nt, Artificial Sequence
  Description of Artificial Sequence: Synthetic oligonucleotide
cuuuu

SEQ ID NO: 2 - RNA, 10 nt, Artificial Sequence
  Description of Artificial Sequence: Synthetic oligonucleotide
aagaccuuuu

SEQ ID NO: 3 - RNA, 25 nt, Artificial Sequence
  Description of Artificial Sequence: Synthetic oligonucleotide
auguguuuuugucaaaagaccuuuu

SEQ ID NO: 4 - RNA, 60 nt, Artificial Sequence
  Description of Artificial Sequence: Synthetic oligonucleotide
aggccagcuugccgguuuuuuagucgugcugcuucauguguuuuugucaaaagaccuuuu

SEQ ID NO: 5 - DNA, 5 bp, Artificial Sequence
  Description of Artificial Sequence: Synthetic oligonucleotide
ctttt

SEQ ID NO: 6 - DNA, 10 bp, Artificial Sequence
  Description of Artificial Sequence: Synthetic oligonucleotide
aagacctttt

SEQ ID NO: 7 - DNA, 25 bp, Artificial Sequence
  Description of Artificial Sequence: Synthetic oligonucleotide
atgtgtttttgtcaaaagacctttt

SEQ ID NO: 8 - DNA, 60 bp, Artificial Sequence
  Description of Artificial Sequence: Synthetic oligonucleotide
aggccagcttgccggttttttagtcgtgctgcttcatgtgtttttgtcaaaagacctttt

SEQ ID NO: 9 - DNA, 20 bp, Artificial Sequence
  Description of Artificial Sequence: Synthetic oligonucleotide
tttttgtcaaaagacctttt

SEQ ID NO: 10 - DNA, 30 bp, Artificial Sequence
  Description of Artificial Sequence: Synthetic oligonucleotide
gcttcatgtgtttttgtcaaaagacctttt

SEQ ID NO: 11 - DNA, 50 bp, Artificial Sequence
  Description of Artificial Sequence: Synthetic oligonucleotide
gccggttttttagtcgtgctgcttcatgtgtttttgtcaaaagacctttt

SEQ ID NO: 12 - DNA, 40 bp, Artificial Sequence
  Description of Artificial Sequence: Synthetic oligonucleotide
tagtcgtgctgcttcatgtgtttttgtcaaaagacctttt

SEQ ID NO: 13 - DNA, 20 bp, Artificial Sequence
  Description of Artificial Sequence: Synthetic oligonucleotide
ccgaagttttcttcggtttt

SEQ ID NO: 14 - DNA, 25 bp, Artificial Sequence
  Description of Artificial Sequence: Synthetic oligonucleotide
tttttccgaagttttcttcggtttt

SEQ ID NO: 15 - DNA, 30 bp, Artificial Sequence
  Description of Artificial Sequence: Synthetic oligonucleotide
aacgctttttccgaagttttcttcggtttt

SEQ ID NO: 16 - DNA, 41 bp, Artificial Sequence
  Description of Artificial Sequence: Synthetic oligonucleotide
gcgttgttttcaacgctttttccgaagttttcttcggtttt

SEQ ID NO: 17 - DNA, 62 bp, Artificial Sequence
  Description of Artificial Sequence: Synthetic oligonucleotide
ggcttcttttgaagcctttttgcgttgttttcaacgctttttccgaagttttcttcggtttt

SEQ ID NO: 18 - DNA, 25 bp, Artificial Sequence
  Description of Artificial Sequence: Synthetic oligonucleotide
atgtgtttttgtcaaaagacctttt

SEQ ID NO: 19 - DNA, 25 bp, Artificial Sequence
  Description of Artificial Sequence: Synthetic oligonucleotide
aaaaaaaaaaaaaaaaaaaaaaaaa

SEQ ID NO: 20 - DNA, 25 bp, Artificial Sequence
  Description of Artificial Sequence: Synthetic oligonucleotide
ttttttttttttttttttttttttt

SEQ ID NO: 21 - DNA, 25 bp, Artificial Sequence
  Description of Artificial Sequence: Synthetic oligonucleotide
  Description of Combined DNA/RNA Molecule: Synthetic oligonucleotide
auguguuuuugucaaaagaccuuuu

SEQ ID NO: 22 - DNA, 25 bp, Artificial Sequence
  Description of Artificial Sequence: Synthetic oligonucleotide
  Description of Combined DNA/RNA Molecule: Synthetic oligonucleotide
aaaaaaaaaaaaaaaaaaaaaaaaa

SEQ ID NO: 23 - RNA, 25 nt, Artificial Sequence
  Description of Artificial Sequence: Synthetic oligonucleotide
uuuuuuuuuuuuuuuuuuuuuuuuu

SEQ ID NO: 24 - DNA, 20 bp, Artificial Sequence
  Description of Artificial Sequence: Synthetic oligonucleotide
tctgcagaaatgttccccgt

SEQ ID NO: 25 - RNA, 20 nt, Artificial Sequence
  Description of Artificial Sequence: Synthetic oligonucleotide
ucugcagaaauguuccccgu

SEQ ID NO: 26 - RNA, 20 nt, Artificial Sequence
  Description of Artificial Sequence: Synthetic oligonucleotide
uaauuucuacucuuguagau

SEQ ID NO: 27 - RNA, 40 nt, Artificial Sequence
  Description of Artificial Sequence: Synthetic oligonucleotide
uaauuucuacucuuguagauucugcagaaauguuccccgu

SEQ ID NO: 28 - DNA, 65 bp, Artificial Sequence
  Description of Artificial Sequence: Synthetic oligonucleotide
  Description of Combined DNA/RNA Molecule: Synthetic oligonucleotide
atgtgtttttgtcaaaagaccttttuaauuucuacucuuguagauucugcagaaauguuccccgu

SEQ ID NO: 29 - DNA, 20 bp, Artificial Sequence
  Description of Artificial Sequence: Synthetic oligonucleotide
caggacgatgtgcagcggcc

SEQ ID NO: 30 - DNA, 20 bp, Artificial Sequence
  Description of Artificial Sequence: Synthetic oligonucleotide
accgcacgttcagaagtcgg

SEQ ID NO: 31 - DNA, 20 bp, Artificial Sequence
  Description of Artificial Sequence: Synthetic oligonucleotide
acaactgtgtaaattttgtg

SEQ ID NO: 32 - DNA, 20 bp, Artificial Sequence
  Description of Artificial Sequence: Synthetic oligonucleotide
caactgtgtaaattttgtga

SEQ ID NO: 33 - DNA, 20 bp, Artificial Sequence
  Description of Artificial Sequence: Synthetic oligonucleotide
acctgtgacaaccagaaatc

SEQ ID NO: 34 - DNA, 20 bp, Artificial Sequence
  Description of Artificial Sequence: Synthetic oligonucleotide
cctgtgacaaccagaaatcc

SEQ ID NO: 35 - DNA, 20 bp, Artificial Sequence
  Description of Artificial Sequence: Synthetic oligonucleotide
tgtggcttctcacagatgga

SEQ ID NO: 36 - DNA, 20 bp, Artificial Sequence
  Description of Artificial Sequence: Synthetic oligonucleotide
tctgtgagaagccacaggaa

SEQ ID NO: 37 - DNA, 20 bp, Artificial Sequence
  Description of Artificial Sequence: Synthetic oligonucleotide
aagctcccctaccatgactt

SEQ ID NO: 38 - DNA, 20 bp, Artificial Sequence
  Description of Artificial Sequence: Synthetic oligonucleotide
gaataaagtcatggtagggg

SEQ ID NO: 39 - DNA, 20 bp, Artificial Sequence
  Description of Artificial Sequence: Synthetic oligonucleotide
agaataaagtcatggtaggg

SEQ ID NO: 40 - DNA, 20 bp, Artificial Sequence
  Description of Artificial Sequence: Synthetic oligonucleotide
ctaccatgactttattctgg

SEQ ID NO: 41 - DNA, 20 bp, Artificial Sequence
  Description of Artificial Sequence: Synthetic oligonucleotide
taccatgactttattctgga

SEQ ID NO: 42 - DNA, 20 bp, Artificial Sequence
  Description of Artificial Sequence: Synthetic oligonucleotide
taatgcactttggagaagca

SEQ ID NO: 43 - DNA, 20 bp, Artificial Sequence
  Description of Artificial Sequence: Synthetic oligonucleotide
ttcataatgcactttggaga

SEQ ID NO: 44 - DNA, 20 bp, Artificial Sequence
  Description of Artificial Sequence: Synthetic oligonucleotide
aagtgcattatgaaggaaaa

SEQ ID NO: 45 - DNA, 20 bp, Artificial Sequence
  Description of Artificial Sequence: Synthetic oligonucleotide
tgtgttcctgtagctctgat

SEQ ID NO: 46 - DNA, 20 bp, Artificial Sequence
  Description of Artificial Sequence: Synthetic oligonucleotide
tgtagctctgatgagtgcaa

SEQ ID NO: 47 - DNA, 20 bp, Artificial Sequence
  Description of Artificial Sequence: Synthetic oligonucleotide
agtgacaggcatcagcctcc

SEQ ID NO: 48 - DNA, 20 bp, Artificial Sequence
  Description of Artificial Sequence: Synthetic oligonucleotide
agtggtggcaggaggctgat

SEQ ID NO: 49 - DNA, 20 bp, Artificial Sequence
  Description of Artificial Sequence: Synthetic oligonucleotide
aggttgaactcagcttctgc

SEQ ID NO: 50 - DNA, 20 bp, Artificial Sequence
  Description of Artificial Sequence: Synthetic oligonucleotide
caggttgaactcagcttctg

SEQ ID NO: 51 - DNA, 20 bp, Artificial Sequence
  Description of Artificial Sequence: Synthetic oligonucleotide
acctgggaaaccggcaagac

SEQ ID NO: 52 - DNA, 20 bp, Artificial Sequence
  Description of Artificial Sequence: Synthetic oligonucleotide
cgtcttgccggtttcccagg

SEQ ID NO: 53 - DNA, 20 bp, Artificial Sequence
  Description of Artificial Sequence: Synthetic oligonucleotide
gcgtcttgccggtttcccag

SEQ ID NO: 54 - DNA, 20 bp, Artificial Sequence
  Description of Artificial Sequence: Synthetic oligonucleotide
tgagcttccgcgtcttgccg

SEQ ID NO: 55 - DNA, 20 bp, Artificial Sequence
  Description of Artificial Sequence: Synthetic oligonucleotide
gcgagcactgtgccatcatc

SEQ ID NO: 56 - DNA, 20 bp, Artificial Sequence
  Description of Artificial Sequence: Synthetic oligonucleotide
ggatgatggcacagtgctcg

SEQ ID NO: 57 - DNA, 20 bp, Artificial Sequence
  Description of Artificial Sequence: Synthetic oligonucleotide
aggatgatggcacagtgctc

SEQ ID NO: 58 - DNA, 20 bp, Artificial Sequence
  Description of Artificial Sequence: Synthetic oligonucleotide
cgtgtgccaacaacatcaac

SEQ ID NO: 59 - DNA, 20 bp, Artificial Sequence
  Description of Artificial Sequence: Synthetic oligonucleotide
gctcaatgggcagcagctct

SEQ ID NO: 60 - DNA, 20 bp, Artificial Sequence
  Description of Artificial Sequence: Synthetic oligonucleotide
accagggtgtccagctcaat

SEQ ID NO: 61 - DNA, 20 bp, Artificial Sequence
  Description of Artificial Sequence: Synthetic oligonucleotide
caccagggtgtccagctcaa

SEQ ID NO: 62 - DNA, 20 bp, Artificial Sequence
  Description of Artificial Sequence: Synthetic oligonucleotide
ccaccagggtgtccagctca

SEQ ID NO: 63 - DNA, 20 bp, Artificial Sequence
  Description of Artificial Sequence: Synthetic oligonucleotide
gcttggccttatagacctca

SEQ ID NO: 64 - DNA, 20 bp, Artificial Sequence
  Description of Artificial Sequence: Synthetic oligonucleotide
gagcagtttgagacagtggc

SEQ ID NO: 65 - DNA, 20 bp, Artificial Sequence
  Description of Artificial Sequence: Synthetic oligonucleotide
agaggcatactcctcatagg

SEQ ID NO: 66 - DNA, 20 bp, Artificial Sequence
  Description of Artificial Sequence: Synthetic oligonucleotide
ctatgaggagtatgcctctt

SEQ ID NO: 67 - DNA, 20 bp, Artificial Sequence
  Description of Artificial Sequence: Synthetic oligonucleotide
aagaggcatactcctcatag

SEQ ID NO: 68 - DNA, 20 bp, Artificial Sequence
  Description of Artificial Sequence: Synthetic oligonucleotide
tatgaggagtatgcctcttg

SEQ ID NO: 69 - DNA, 20 bp, Artificial Sequence
  Description of Artificial Sequence: Synthetic oligonucleotide
gattgatgtctgagaagatg

SEQ ID NO: 70 - DNA, 20 bp, Artificial Sequence
  Description of Artificial Sequence: Synthetic oligonucleotide
ctcctcagccgtcaggaact

SEQ ID NO: 71 - DNA, 20 bp, Artificial Sequence
  Description of Artificial Sequence: Synthetic oligonucleotide
gttcctgacggctgaggagc

SEQ ID NO: 72 - DNA, 20 bp, Artificial Sequence
  Description of Artificial Sequence: Synthetic oligonucleotide
gctcctcagccgtcaggaac

SEQ ID NO: 73 - DNA, 20 bp, Artificial Sequence
  Description of Artificial Sequence: Synthetic oligonucleotide
tgacggctgaggagcggaag

SEQ ID NO: 74 - DNA, 20 bp, Artificial Sequence
  Description of Artificial Sequence: Synthetic oligonucleotide
tcttccgctcctcagccgtc

SEQ ID NO: 75 - DNA, 20 bp, Artificial Sequence
  Description of Artificial Sequence: Synthetic oligonucleotide
aactccgtcttccgctcctc

SEQ ID NO: 76 - DNA, 20 bp, Artificial Sequence
  Description of Artificial Sequence: Synthetic oligonucleotide
caactccgtcttccgctcct

SEQ ID NO: 77 - DNA, 20 bp, Artificial Sequence
  Description of Artificial Sequence: Synthetic oligonucleotide
ccaactccgtcttccgctcc

SEQ ID NO: 78 - DNA, 20 bp, Artificial Sequence
  Description of Artificial Sequence: Synthetic oligonucleotide
acgccaagggcaacctacag

SEQ ID NO: 79 - DNA, 20 bp, Artificial Sequence
  Description of Artificial Sequence: Synthetic oligonucleotide
cgccaagggcaacctacagg

SEQ ID NO: 80 - DNA, 20 bp, Artificial Sequence
  Description of Artificial Sequence: Synthetic oligonucleotide
agctgatgacatgccgcgtc

SEQ ID NO: 81 - DNA, 20 bp, Artificial Sequence
  Description of Artificial Sequence: Synthetic oligonucleotide
gggcgagggagctgcccagc

SEQ ID NO: 82 - DNA, 20 bp, Artificial Sequence
  Description of Artificial Sequence: Synthetic oligonucleotide
cgggcgagggagctgcccag

SEQ ID NO: 83 - DNA, 20 bp, Artificial Sequence
  Description of Artificial Sequence: Synthetic oligonucleotide
ccgggcgagggagctgccca

SEQ ID NO: 84 - DNA, 20 bp, Artificial Sequence
  Description of Artificial Sequence: Synthetic oligonucleotide
tcgcccgggggattgctcac

SEQ ID NO: 85 - DNA, 20 bp, Artificial Sequence
  Description of Artificial Sequence: Synthetic oligonucleotide
acatggagtgtgatcactgt

SEQ ID NO: 86 - DNA, 20 bp, Artificial Sequence
  Description of Artificial Sequence: Synthetic oligonucleotide
cagtgatcacactccatgtg

SEQ ID NO: 87 - DNA, 20 bp, Artificial Sequence
  Description of Artificial Sequence: Synthetic oligonucleotide
tgtgggaggcccaagatgcc

SEQ ID NO: 88 - DNA, 20 bp, Artificial Sequence
  Description of Artificial Sequence: Synthetic oligonucleotide
tgtgcacgatgggcatcttg

SEQ ID NO: 89 - DNA, 20 bp, Artificial Sequence
  Description of Artificial Sequence: Synthetic oligonucleotide
cgaggatattggagctcttg

SEQ ID NO: 90 - DNA, 20 bp, Artificial Sequence
  Description of Artificial Sequence: Synthetic oligonucleotide
atatcctcgtgaagaacgac

SEQ ID NO: 91 - DNA, 20 bp, Artificial Sequence
  Description of Artificial Sequence: Synthetic oligonucleotide
gacgcagggaaagcccaaag

SEQ ID NO: 92 - DNA, 20 bp, Artificial Sequence
  Description of Artificial Sequence: Synthetic oligonucleotide
ctgcgtctggaccctactct

SEQ ID NO: 93 - DNA, 20 bp, Artificial Sequence
  Description of Artificial Sequence: Synthetic oligonucleotide
tgcgtctggaccctactctg

SEQ ID NO: 94 - DNA, 20 bp, Artificial Sequence
  Description of Artificial Sequence: Synthetic oligonucleotide
cagacagagtagggtccaga

SEQ ID NO: 95 - DNA, 20 bp, Artificial Sequence
  Description of Artificial Sequence: Synthetic oligonucleotide
gccagcacgatcccaccgca

SEQ ID NO: 96 - DNA, 20 bp, Artificial Sequence
  Description of Artificial Sequence: Synthetic oligonucleotide
aaggaaaaaaaaaagcctgg

SEQ ID NO: 97 - DNA, 20 bp, Artificial Sequence
  Description of Artificial Sequence: Synthetic oligonucleotide
acaccagcaatcctgacttg

SEQ ID NO: 98 - DNA, 20 bp, Artificial Sequence
  Description of Artificial Sequence: Synthetic oligonucleotide
actagcaacaagtcaggatt

SEQ ID NO: 99 - DNA, 20 bp, Artificial Sequence
  Description of Artificial Sequence: Synthetic oligonucleotide
gcaactcccagtggtggcag

SEQ ID NO: 100 - DNA, 20 bp, Artificial Sequence
  Description of Artificial Sequence: Synthetic oligonucleotide
tgtcatcatcatcttctact

SEQ ID NO: 101 - DNA, 20 bp, Artificial Sequence
  Description of Artificial Sequence: Synthetic oligonucleotide
gacctcagcaaagcgacctt

SEQ ID NO: 102 - DNA, 20 bp, Artificial Sequence
  Description of Artificial Sequence: Synthetic oligonucleotide
aggccaagctgaagcagaac

SEQ ID NO: 103 - DNA, 20 bp, Artificial Sequence
  Description of Artificial Sequence: Synthetic oligonucleotide
aggagtatgcctcttggaag

SEQ ID NO: 104 - DNA, 20 bp, Artificial Sequence
  Description of Artificial Sequence: Synthetic oligonucleotide
cctcttggaagacagagaag

SEQ ID NO: 105 - DNA, 20 bp, Artificial Sequence
  Description of Artificial Sequence: Synthetic oligonucleotide
ttctcatgcttcagattgat

SEQ ID NO: 106 - DNA, 20 bp, Artificial Sequence
  Description of Artificial Sequence: Synthetic oligonucleotide
ctcgtgaagaacgacctaac

SEQ ID NO: 107 - DNA, 20 bp, Artificial Sequence
  Description of Artificial Sequence: Synthetic oligonucleotide
ggccgctgcacatcgtcctg

SEQ ID NO: 108 - DNA, 20 bp, Artificial Sequence
  Description of Artificial Sequence: Synthetic oligonucleotide
gcggggtctgccatgggtcg

SEQ ID NO: 109 - DNA, 20 bp, Artificial Sequence
  Description of Artificial Sequence: Synthetic oligonucleotide
agttgctcatgcaggatttc

SEQ ID NO: 110 - DNA, 20 bp, Artificial Sequence
  Description of Artificial Sequence: Synthetic oligonucleotide
ccagaataaagtcatggtag

SEQ ID NO: 111 - DNA, 20 bp, Artificial Sequence
  Description of Artificial Sequence: Synthetic oligonucleotide
cccctaccatgactttattc

SEQ ID NO: 112 - DNA, 20 bp, Artificial Sequence
  Description of Artificial Sequence: Synthetic oligonucleotide
aagtcatggtaggggagctt

SEQ ID NO: 113 - DNA, 20 bp, Artificial Sequence
  Description of Artificial Sequence: Synthetic oligonucleotide
agtcatggtaggggagcttg

SEQ ID NO: 114 - DNA, 20 bp, Artificial Sequence
  Description of Artificial Sequence: Synthetic oligonucleotide
attgcactcatcagagctac

SEQ ID NO: 115 - DNA, 20 bp, Artificial Sequence
  Description of Artificial Sequence: Synthetic oligonucleotide
cctagagtgaagagattcat

SEQ ID NO: 116 - DNA, 20 bp, Artificial Sequence
  Description of Artificial Sequence: Synthetic oligonucleotide
ccaatgaatctcttcactct

SEQ ID NO: 117 - DNA, 20 bp, Artificial Sequence
  Description of Artificial Sequence: Synthetic oligonucleotide
aaagtcatggtaggggagct

SEQ ID NO: 118 - DNA, 20 bp, Artificial Sequence
  Description of Artificial Sequence: Synthetic oligonucleotide
gtgagcaatcccccgggcga

SEQ ID NO: 119 - DNA, 20 bp, Artificial Sequence
  Description of Artificial Sequence: Synthetic oligonucleotide
gtcgttcttcacgaggatat

SEQ ID NO: 120 - DNA, 20 bp, Artificial Sequence
  Description of Artificial Sequence: Synthetic oligonucleotide
gccgcgtcaggtactcctgt

SEQ ID NO: 121 - DNA, 20 bp, Artificial Sequence
  Description of Artificial Sequence: Synthetic oligonucleotide
gacgcggcatgtcatcagct

SEQ ID NO: 122 - DNA, 20 bp, Artificial Sequence
  Description of Artificial Sequence: Synthetic oligonucleotide
gcttctgctgccggttaacg

SEQ ID NO: 123 - DNA, 20 bp, Artificial Sequence
  Description of Artificial Sequence: Synthetic oligonucleotide
gtggatgacctggctaacag

SEQ ID NO: 124 - DNA, 20 bp, Artificial Sequence
  Description of Artificial Sequence: Synthetic oligonucleotide
gtgatcacactccatgtggg

SEQ ID NO: 125 - DNA, 20 bp, Artificial Sequence
  Description of Artificial Sequence: Synthetic oligonucleotide
gcccattgagctggacaccc

SEQ ID NO: 126 - DNA, 20 bp, Artificial Sequence
  Description of Artificial Sequence: Synthetic oligonucleotide
gcggtcatcttccaggatga

SEQ ID NO: 127 - DNA, 20 bp, Artificial Sequence
  Description of Artificial Sequence: Synthetic oligonucleotide
gggagctgcccagcttgcgc

SEQ ID NO: 128 - DNA, 20 bp, Artificial Sequence
  Description of Artificial Sequence: Synthetic oligonucleotide
gttgatgttgttggcacacg

SEQ ID NO: 129 - DNA, 20 bp, Artificial Sequence
  Description of Artificial Sequence: Synthetic oligonucleotide
ggcatcttgggcctcccaca

SEQ ID NO: 130 - DNA, 20 bp, Artificial Sequence
  Description of Artificial Sequence: Synthetic oligonucleotide
gcggcatgtcatcagctggg

SEQ ID NO: 131 - DNA, 20 bp, Artificial Sequence
  Description of Artificial Sequence: Synthetic oligonucleotide
gctcctcagccgtcaggaac

SEQ ID NO: 132 - DNA, 20 bp, Artificial Sequence
  Description of Artificial Sequence: Synthetic oligonucleotide
gctggtgttatattctgatg

SEQ ID NO: 133 - DNA, 20 bp, Artificial Sequence
  Description of Artificial Sequence: Synthetic oligonucleotide
ccgacttctgaacgtgcggt

SEQ ID NO: 134 - DNA, 20 bp, Artificial Sequence
  Description of Artificial Sequence: Synthetic oligonucleotide
tgctggcgatacgcgtccac

SEQ ID NO: 135 - DNA, 20 bp, Artificial Sequence
  Description of Artificial Sequence: Synthetic oligonucleotide
cccgacttctgaacgtgcgg

SEQ ID NO: 136 - DNA, 20 bp, Artificial Sequence
  Description of Artificial Sequence: Synthetic oligonucleotide
ccaccgcacgttcagaagtc

SEQ ID NO: 137 - DNA, 20 bp, Artificial Sequence
  Description of Artificial Sequence: Synthetic oligonucleotide
tcacccgacttctgaacgtg

SEQ ID NO: 138 - DNA, 20 bp, Artificial Sequence
  Description of Artificial Sequence: Synthetic oligonucleotide
cccaccgcacgttcagaagt

SEQ ID NO: 139 - DNA, 20 bp, Artificial Sequence
  Description of Artificial Sequence: Synthetic oligonucleotide
cgagcagcggggtctgccat

SEQ ID NO: 140 - DNA, 20 bp, Artificial Sequence
  Description of Artificial Sequence: Synthetic oligonucleotide
acgagcagcggggtctgcca

SEQ ID NO: 141 - DNA, 20 bp, Artificial Sequence
  Description of Artificial Sequence: Synthetic oligonucleotide
agcggggtctgccatgggtc

SEQ ID NO: 142 - DNA, 20 bp, Artificial Sequence
  Description of Artificial Sequence: Synthetic oligonucleotide
cctgagcagcccccgaccca

SEQ ID NO: 143 - DNA, 20 bp, Artificial Sequence
  Description of Artificial Sequence: Synthetic oligonucleotide
ccatgggtcgggggctgctc

SEQ ID NO: 144 - DNA, 20 bp, Artificial Sequence
  Description of Artificial Sequence: Synthetic oligonucleotide
aacgtgcggtgggatcgtgc

SEQ ID NO: 145 - DNA, 20 bp, Artificial Sequence
  Description of Artificial Sequence: Synthetic oligonucleotide
ggacgatgtgcagcggccac

SEQ ID NO: 146 - DNA, 20 bp, Artificial Sequence
  Description of Artificial Sequence: Synthetic oligonucleotide
gtccacaggacgatgtgcag

SEQ ID NO: 147 - DNA, 20 bp, Artificial Sequence
  Description of Artificial Sequence: Synthetic oligonucleotide
catgggtcgggggctgctca

SEQ ID NO: 148 - DNA, 20 bp, Artificial Sequence
  Description of Artificial Sequence: Synthetic oligonucleotide
cagcggggtctgccatgggt

SEQ ID NO: 149 - DNA, 20 bp, Artificial Sequence
  Description of Artificial Sequence: Synthetic oligonucleotide
atgggtcgggggctgctcag

SEQ ID NO: 150 - DNA, 20 bp, Artificial Sequence
  Description of Artificial Sequence: Synthetic oligonucleotide
cggggtctgccatgggtcgg

SEQ ID NO: 151 - DNA, 20 bp, Artificial Sequence
  Description of Artificial Sequence: Synthetic oligonucleotide
aggaagtctgtgtggctgta

SEQ ID NO: 152 - DNA, 20 bp, Artificial Sequence
  Description of Artificial Sequence: Synthetic oligonucleotide
ctccatctgtgagaagccac

SEQ ID NO: 153 - DNA, 20 bp, Artificial Sequence
  Description of Artificial Sequence: Synthetic oligonucleotide
atgatagtcactgacaacaa

SEQ ID NO: 154 - DNA, 20 bp, Artificial Sequence
  Description of Artificial Sequence: Synthetic oligonucleotide
gatgctgcagttgctcatgc

SEQ ID NO: 155 - DNA, 20 bp, Artificial Sequence
  Description of Artificial Sequence: Synthetic oligonucleotide
acagccacacagacttcctg

SEQ ID NO: 156 - DNA, 20 bp, Artificial Sequence
  Description of Artificial Sequence: Synthetic oligonucleotide
gaagccacaggaagtctgtg

SEQ ID NO: 157 - DNA, 20 bp, Artificial Sequence
  Description of Artificial Sequence: Synthetic oligonucleotide
ttcctgtggcttctcacaga

SEQ ID NO: 158 - DNA, 20 bp, Artificial Sequence
  Description of Artificial Sequence: Synthetic oligonucleotide
ctgtggcttctcacagatgg

SEQ ID NO: 159 - DNA, 20 bp, Artificial Sequence
  Description of Artificial Sequence: Synthetic oligonucleotide
tcacaaaatttacacagttg

SEQ ID NO: 160 - DNA, 20 bp, Artificial Sequence
  Description of Artificial Sequence: Synthetic oligonucleotide
gacaacatcatcttctcaga

SEQ ID NO: 161 - DNA, 20 bp, Artificial Sequence
  Description of Artificial Sequence: Synthetic oligonucleotide
tccagaataaagtcatggta

SEQ ID NO: 162 - DNA, 20 bp, Artificial Sequence
  Description of Artificial Sequence: Synthetic oligonucleotide
ggtaggggagcttggggtca

SEQ ID NO: 163 - DNA, 20 bp, Artificial Sequence
  Description of Artificial Sequence: Synthetic oligonucleotide
ttctccaaagtgcattatga

SEQ ID NO: 164 - DNA, 20 bp, Artificial Sequence
  Description of Artificial Sequence: Synthetic oligonucleotide
catcttccagaataaagtca

SEQ ID NO: 165 - DNA, 20 bp, Artificial Sequence
  Description of Artificial Sequence: Synthetic oligonucleotide
cacatgaagaaagtctcacc

SEQ ID NO: 166 - DNA, 20 bp, Artificial Sequence
  Description of Artificial Sequence: Synthetic oligonucleotide
ttccagaataaagtcatggt

SEQ ID NO: 167 - DNA, 20 bp, Artificial Sequence
  Description of Artificial Sequence: Synthetic oligonucleotide
ttttccttcataatgcactt

SEQ ID NO: 168 - DNA, 20 bp, Artificial Sequence
  Description of Artificial Sequence: Synthetic oligonucleotide
cacagttgtggaaacttgac

SEQ ID NO: 169 - DNA, 20 bp, Artificial Sequence
  Description of Artificial Sequence: Synthetic oligonucleotide
cccaactccgtcttccgctc

SEQ ID NO: 170 - DNA, 20 bp, Artificial Sequence
  Description of Artificial Sequence: Synthetic oligonucleotide
ggctttccctgcgtctggac

SEQ ID NO: 171 - DNA, 20 bp, Artificial Sequence
  Description of Artificial Sequence: Synthetic oligonucleotide
ctgaggtctataaggccaag

SEQ ID NO: 172 - DNA, 20 bp, Artificial Sequence
  Description of Artificial Sequence: Synthetic oligonucleotide
tgatgtgagattttccacct

SEQ ID NO: 173 - DNA, 20 bp, Artificial Sequence
  Description of Artificial Sequence: Synthetic oligonucleotide
cctatgaggagtatgcctct

SEQ ID NO: 174 - DNA, 20 bp, Artificial Sequence
  Description of Artificial Sequence: Synthetic oligonucleotide
aagtgacaggcatcagcctc

SEQ ID NO: 175 - DNA, 20 bp, Artificial Sequence
  Description of Artificial Sequence: Synthetic oligonucleotide
ccatgaccccaagctcccct

SEQ ID NO: 176 - DNA, 20 bp, Artificial Sequence
  Description of Artificial Sequence: Synthetic oligonucleotide
cttcataatgcactttggag

SEQ ID NO: 177 - DNA, 20 bp, Artificial Sequence
  Description of Artificial Sequence: Synthetic oligonucleotide
ttcatgtgttcctgtagctc

SEQ ID NO: 178 - DNA, 20 bp, Artificial Sequence
  Description of Artificial Sequence: Synthetic oligonucleotide
ttctggaagatgctgcttct

SEQ ID NO: 179 - DNA, 20 bp, Artificial Sequence
  Description of Artificial Sequence: Synthetic oligonucleotide
cccaccagggtgtccagctc

SEQ ID NO: 180 - DNA, 20 bp, Artificial Sequence
  Description of Artificial Sequence: Synthetic oligonucleotide
agacagtggcagtcaagatc

SEQ ID NO: 181 - DNA, 20 bp, Artificial Sequence
  Description of Artificial Sequence: Synthetic oligonucleotide
cctgcgtctggaccctactc

SEQ ID NO: 182 - DNA, 20 bp, Artificial Sequence
  Description of Artificial Sequence: Synthetic oligonucleotide
cacaactgtgtaaattttgt

SEQ ID NO: 183 - DNA, 20 bp, Artificial Sequence
  Description of Artificial Sequence: Synthetic oligonucleotide
gagaagcagcatcttccaga

SEQ ID NO: 184 - DNA, 20 bp, Artificial Sequence
  Description of Artificial Sequence: Synthetic oligonucleotide
tggttgtcacaggtggaaaa

SEQ ID NO: 185 - DNA, 20 bp, Artificial Sequence
  Description of Artificial Sequence: Synthetic oligonucleotide
ccaggttgaactcagcttct

SEQ ID NO: 186 - DNA, 21 bp, Artificial Sequence
  Description of Artificial Sequence: Synthetic oligonucleotide
atcacaaaatttacacagttg

SEQ ID NO: 187 - DNA, 21 bp, Artificial Sequence
  Description of Artificial Sequence: Synthetic oligonucleotide
ggcatcagcctcctgccacca

SEQ ID NO: 188 - DNA, 21 bp, Artificial Sequence
  Description of Artificial Sequence: Synthetic oligonucleotide
gttagccaggtcatccacaga

SEQ ID NO: 189 - DNA, 21 bp, Artificial Sequence
  Description of Artificial Sequence: Synthetic oligonucleotide
gctgggcagctccctcgcccg

SEQ ID NO: 190 - DNA, 21 bp, Artificial Sequence
  Description of Artificial Sequence: Synthetic oligonucleotide
caggaggctgatgcctgtcac

SEQ ID NO: 191 - DNA, 21 bp, Artificial Sequence
  Description of Artificial Sequence: Synthetic oligonucleotide
gaggagcggaagacggagttg

SEQ ID NO: 192 - DNA, 21 bp, Artificial Sequence
  Description of Artificial Sequence: Synthetic oligonucleotide
cgtctggaccctactctgtct

SEQ ID NO: 193 - DNA, 21 bp, Artificial Sequence
  Description of Artificial Sequence: Synthetic oligonucleotide
tttttccttcataatgcactt

SEQ ID NO: 194 - DNA, 21 bp, Artificial Sequence
  Description of Artificial Sequence: Synthetic oligonucleotide
ccattgagctggacaccctgg

SEQ ID NO: 195 - DNA, 21 bp, Artificial Sequence
  Description of Artificial Sequence: Synthetic oligonucleotide
cttctccaaagtgcattatga

SEQ ID NO: 196 - DNA, 21 bp, Artificial Sequence
  Description of Artificial Sequence: Synthetic oligonucleotide
gcccaagatgcccatcgtgca

SEQ ID NO: 197 - DNA, 21 bp, Artificial Sequence
  Description of Artificial Sequence: Synthetic oligonucleotide
tcatgtgttcctgtagctctg

SEQ ID NO: 198 - DNA, 21 bp, Artificial Sequence
  Description of Artificial Sequence: Synthetic oligonucleotide
gtgatgctgcagttgctcatg

SEQ ID NO: 199 - DNA, 21 bp, Artificial Sequence
  Description of Artificial Sequence: Synthetic oligonucleotide
tctcatgcttcagattgatgt

SEQ ID NO: 200 - DNA, 21 bp, Artificial Sequence
  Description of Artificial Sequence: Synthetic oligonucleotide
tccctatgaggagtatgcctc

SEQ ID NO: 201 - DNA, 21 bp, Artificial Sequence
  Description of Artificial Sequence: Synthetic oligonucleotide
catcacaaaatttacacagtt

SEQ ID NO: 202 - DNA, 21 bp, Artificial Sequence
  Description of Artificial Sequence: Synthetic oligonucleotide
attgagctggacaccctggtg

SEQ ID NO: 203 - DNA, 21 bp, Artificial Sequence
  Description of Artificial Sequence: Synthetic oligonucleotide
cagtcaagatctttccctatg

SEQ ID NO: 204 - DNA, 21 bp, Artificial Sequence
  Description of Artificial Sequence: Synthetic oligonucleotide
aggatttctggttgtcacagg

SEQ ID NO: 205 - DNA, 21 bp, Artificial Sequence
  Description of Artificial Sequence: Synthetic oligonucleotide
tccacagtgatcacactccat

SEQ ID NO: 206 - DNA, 21 bp, Artificial Sequence
  Description of Artificial Sequence: Synthetic oligonucleotide
agcagaacacttcagagcagt

SEQ ID NO: 207 - DNA, 21 bp, Artificial Sequence
  Description of Artificial Sequence: Synthetic oligonucleotide
ccggcaagacgcggaagctca

SEQ ID NO: 208 - DNA, 21 bp, Artificial Sequence
  Description of Artificial Sequence: Synthetic oligonucleotide
gatgtcagagcggtcatcttc

SEQ ID NO: 209 - DNA, 21 bp, Artificial Sequence
  Description of Artificial Sequence: Synthetic oligonucleotide
tcattgcactcatcagagcta

SEQ ID NO: 210 - DNA, 21 bp, Artificial Sequence
  Description of Artificial Sequence: Synthetic oligonucleotide
cttccagaataaagtcatggt

SEQ ID NO: 211 - DNA, 21 bp, Artificial Sequence
  Description of Artificial Sequence: Synthetic oligonucleotide
agattttccacctgtgacaac

SEQ ID NO: 212 - DNA, 21 bp, Artificial Sequence
  Description of Artificial Sequence: Synthetic oligonucleotide
actgcagcatcacctccatct

SEQ ID NO: 213 - DNA, 21 bp, Artificial Sequence
  Description of Artificial Sequence: Synthetic oligonucleotide
agctgggcagctccctcgccc

SEQ ID NO: 214 - DNA, 21 bp, Artificial Sequence
  Description of Artificial Sequence: Synthetic oligonucleotide
tgacggctgaggagcggaaga

SEQ ID NO: 215 - DNA, 21 bp, Artificial Sequence
  Description of Artificial Sequence: Synthetic oligonucleotide
cattgagctggacaccctggt

SEQ ID NO: 216 - DNA, 21 bp, Artificial Sequence
  Description of Artificial Sequence: Synthetic oligonucleotide
agcaaagcgacctttccccac

SEQ ID NO: 217 - DNA, 21 bp, Artificial Sequence
  Description of Artificial Sequence: Synthetic oligonucleotide
cgcgttaaccggcagcagaag

SEQ ID NO: 218 - DNA, 21 bp, Artificial Sequence
  Description of Artificial Sequence: Synthetic oligonucleotide
gaaatatgactagcaacaagt

SEQ ID NO: 219 - DNA, 21 bp, Artificial Sequence
  Description of Artificial Sequence: Synthetic oligonucleotide
agacagagtagggtccagacg

SEQ ID NO: 220 - DNA, 21 bp, Artificial Sequence
  Description of Artificial Sequence: Synthetic oligonucleotide
caggatttctggttgtcacag

SEQ ID NO: 221 - DNA, 21 bp, Artificial Sequence
  Description of Artificial Sequence: Synthetic oligonucleotide
ctcctgtaggttgcccttggc

SEQ ID NO: 222 - DNA, 21 bp, Artificial Sequence
  Description of Artificial Sequence: Synthetic oligonucleotide
acagagtagggtccagacgca

SEQ ID NO: 223 - DNA, 21 bp, Artificial Sequence
  Description of Artificial Sequence: Synthetic oligonucleotide
gcttctccaaagtgcattatg

SEQ ID NO: 224 - DNA, 21 bp, Artificial Sequence
  Description of Artificial Sequence: Synthetic oligonucleotide
gcagcagaagctgagttcaac

SEQ ID NO: 225 - DNA, 21 bp, Artificial Sequence
  Description of Artificial Sequence: Synthetic oligonucleotide
tgaggagcggaagacggagtt

SEQ ID NO: 226 - DNA, 21 bp, Artificial Sequence
  Description of Artificial Sequence: Synthetic oligonucleotide
ctttggagaagcagcatcttc

SEQ ID NO: 227 - DNA, 21 bp, Artificial Sequence
  Description of Artificial Sequence: Synthetic oligonucleotide
ctcccctaccatgactttatt

SEQ ID NO: 228 - DNA, 21 bp, Artificial Sequence
  Description of Artificial Sequence: Synthetic oligonucleotide
gacagagtagggtccagacgc

SEQ ID NO: 229 - DNA, 21 bp, Artificial Sequence
  Description of Artificial Sequence: Synthetic oligonucleotide
ctgaggagcggaagacggagt

SEQ ID NO: 230 - DNA, 21 bp, Artificial Sequence
  Description of Artificial Sequence: Synthetic oligonucleotide
gggcatcttgggcctcccaca

SEQ ID NO: 231 - DNA, 21 bp, Artificial Sequence
  Description of Artificial Sequence: Synthetic oligonucleotide
ccaagaggcatactcctcata

SEQ ID NO: 232 - DNA, 21 bp, Artificial Sequence
  Description of Artificial Sequence: Synthetic oligonucleotide
agaatgacgagaacataacac

SEQ ID NO: 233 - DNA, 21 bp, Artificial Sequence
  Description of Artificial Sequence: Synthetic oligonucleotide
cctgacgcggcatgtcatcag

SEQ ID NO: 234 - DNA, 21 bp, Artificial Sequence
  Description of Artificial Sequence: Synthetic oligonucleotide
agcgagcactgtgccatcatc

SEQ ID NO: 235 - DNA, 21 bp, Artificial Sequence
  Description of Artificial Sequence: Synthetic oligonucleotide
gcaggttaggtcgttcttcac

SEQ ID NO: 236 - DNA, 21 bp, Artificial Sequence
  Description of Artificial Sequence: Synthetic oligonucleotide
acctccatctgtgagaagcca

SEQ ID NO: 237 - DNA, 21 bp, Artificial Sequence
  Description of Artificial Sequence: Synthetic oligonucleotide
taaagtcatggtaggggagct

SEQ ID NO: 238 - DNA, 21 bp, Artificial Sequence
  Description of Artificial Sequence: Synthetic oligonucleotide
tcagagctacaggaacacatg

SEQ ID NO: 239 - DNA, 21 bp, Artificial Sequence
  Description of Artificial Sequence: Synthetic oligonucleotide
tctcagacatcaatctgaagc

SEQ ID NO: 240 - DNA, 21 bp, Artificial Sequence
  Description of Artificial Sequence: Synthetic oligonucleotide
catcagcctcctgccaccact

SEQ ID NO: 241 - DNA, 21 bp, Artificial Sequence
  Description of Artificial Sequence: Synthetic oligonucleotide
cgctcctcagccgtcaggaac

SEQ ID NO: 242 - DNA, 21 bp, Artificial Sequence
  Description of Artificial Sequence: Synthetic oligonucleotide
aacctgggaaaccggcaagac

SEQ ID NO: 243 - DNA, 21 bp, Artificial Sequence
  Description of Artificial Sequence: Synthetic oligonucleotide
tccacgccaagggcaacctac

SEQ ID NO: 244 - DNA, 21 bp, Artificial Sequence
  Description of Artificial Sequence: Synthetic oligonucleotide
gaggtgagcaatcccccgggc

SEQ ID NO: 245 - DNA, 21 bp, Artificial Sequence
  Description of Artificial Sequence: Synthetic oligonucleotide
cagcagaagctgagttcaacc

SEQ ID NO: 246 - DNA, 21 bp, Artificial Sequence
  Description of Artificial Sequence: Synthetic oligonucleotide
tccaagaggcatactcctcat

SEQ ID NO: 247 - DNA, 21 bp, Artificial Sequence
  Description of Artificial Sequence: Synthetic oligonucleotide
agcagaagctgagttcaacct

SEQ ID NO: 248 - DNA, 21 bp, Artificial Sequence
  Description of Artificial Sequence: Synthetic oligonucleotide
ccagttcctgacggctgagga

SEQ ID NO: 249 - DNA, 21 bp, Artificial Sequence
  Description of Artificial Sequence: Synthetic oligonucleotide
aggagtatgcctcttggaaga

SEQ ID NO: 250 - DNA, 21 bp, Artificial Sequence
  Description of Artificial Sequence: Synthetic oligonucleotide
ttccaagaggcatactcctca

SEQ ID NO: 251 - DNA, 21 bp, Artificial Sequence
  Description of Artificial Sequence: Synthetic oligonucleotide
caactgtgtaaattttgtgat

SEQ ID NO: 252 - DNA, 21 bp, Artificial Sequence
  Description of Artificial Sequence: Synthetic oligonucleotide
tgaaggaaaaaaaaaagcctg

SEQ ID NO: 253 - DNA, 21 bp, Artificial Sequence
  Description of Artificial Sequence: Synthetic oligonucleotide
cgtcttccgctcctcagccgt

SEQ ID NO: 254 - DNA, 21 bp, Artificial Sequence
  Description of Artificial Sequence: Synthetic oligonucleotide
ccaggtcatccacagacagag

SEQ ID NO: 255 - DNA, 21 bp, Artificial Sequence
  Description of Artificial Sequence: Synthetic oligonucleotide
gcctagagtgaagagattcat

SEQ ID NO: 256 - DNA, 21 bp, Artificial Sequence
  Description of Artificial Sequence: Synthetic oligonucleotide
gttctccaaagtgcattatga

SEQ ID NO: 257 - DNA, 21 bp, Artificial Sequence
  Description of Artificial Sequence: Synthetic oligonucleotide
gcatcttccagaataaagtca

SEQ ID NO: 258 - DNA, 20 bp, Artificial Sequence
  Description of Artificial Sequence: Synthetic oligonucleotide
caaccgtctggtggccgacg

SEQ ID NO: 259 - DNA, 20 bp, Artificial Sequence
  Description of Artificial Sequence: Synthetic oligonucleotide
caggatcggggctgtcgctt

SEQ ID NO: 260 - DNA, 20 bp, Artificial Sequence
  Description of Artificial Sequence: Synthetic oligonucleotide
tcgggcctcgctggccgtaa

SEQ ID NO: 261 - DNA, 20 bp, Artificial Sequence
  Description of Artificial Sequence: Synthetic oligonucleotide
gaggtagtcggccatgcgcc

SEQ ID NO: 262 - DNA, 20 bp, Artificial Sequence
  Description of Artificial Sequence: Synthetic oligonucleotide
caggtgttgtcgggcctcgc

SEQ ID NO: 263 - DNA, 20 bp, Artificial Sequence
  Description of Artificial Sequence: Synthetic oligonucleotide
ggaggtagtcggccatgcgc

SEQ ID NO: 264 - DNA, 20 bp, Artificial Sequence
  Description of Artificial Sequence: Synthetic oligonucleotide
ggcatactcaatgcgtacat

SEQ ID NO: 265 - DNA, 20 bp, Artificial Sequence
  Description of Artificial Sequence: Synthetic oligonucleotide
ccgccttgtcatcaaccgtc

SEQ ID NO: 266 - DNA, 20 bp, Artificial Sequence
  Description of Artificial Sequence: Synthetic oligonucleotide
aggatcggggctgtcgcttc

SEQ ID NO: 267 - DNA, 20 bp, Artificial Sequence
  Description of Artificial Sequence: Synthetic oligonucleotide
ccttgtcatcaaccgtctgg

SEQ ID NO: 268 - DNA, 20 bp, Artificial Sequence
  Description of Artificial Sequence: Synthetic oligonucleotide
tactcaatgcgtacattggt

SEQ ID NO: 269 - DNA, 20 bp, Artificial Sequence
  Description of Artificial Sequence: Synthetic oligonucleotide
gggttccattacggccagcg

SEQ ID NO: 270 - DNA, 20 bp, Artificial Sequence
  Description of Artificial Sequence: Synthetic oligonucleotide
ggcactgcttctgcgtacaa

SEQ ID NO: 271 - DNA, 20 bp, Artificial Sequence
  Description of Artificial Sequence: Synthetic oligonucleotide
ggttgatgacaaggcggcac

SEQ ID NO: 272 - DNA, 20 bp, Artificial Sequence
  Description of Artificial Sequence: Synthetic oligonucleotide
tgctggggccttcctcgagg

SEQ ID NO: 273 - DNA, 20 bp, Artificial Sequence
  Description of Artificial Sequence: Synthetic oligonucleotide
ttgctggctgtggagcggac

SEQ ID NO: 274 - DNA, 20 bp, Artificial Sequence
  Description of Artificial Sequence: Synthetic oligonucleotide
ttctcctaccttcgggaatc

SEQ ID NO: 275 - DNA, 20 bp, Artificial Sequence
  Description of Artificial Sequence: Synthetic oligonucleotide
gactggcttgggcagttcca

SEQ ID NO: 276 - DNA, 20 bp, Artificial Sequence
  Description of Artificial Sequence: Synthetic oligonucleotide
catgcagcccttgcctgctg

SEQ ID NO: 277 - DNA, 20 bp, Artificial Sequence
  Description of Artificial Sequence: Synthetic oligonucleotide
agcaaaggacgaggtctaga

SEQ ID NO: 278 - DNA, 20 bp, Artificial Sequence
  Description of Artificial Sequence: Synthetic oligonucleotide
gcctgctggggccttcctcg

SEQ ID NO: 279 - DNA, 20 bp, Artificial Sequence
  Description of Artificial Sequence: Synthetic oligonucleotide
cagactcaccagattcccga

SEQ ID NO: 280 - DNA, 20 bp, Artificial Sequence
  Description of Artificial Sequence: Synthetic oligonucleotide
acctcgtcctttgctggctg

SEQ ID NO: 281 - DNA, 20 bp, Artificial Sequence
  Description of Artificial Sequence: Synthetic oligonucleotide
ctcaccagattcccgaaggt

SEQ ID NO: 282 - DNA, 20 bp, Artificial Sequence
  Description of Artificial Sequence: Synthetic oligonucleotide
tacgcagaagcagtgcccgc

SEQ ID NO: 283 - DNA, 20 bp, Artificial Sequence
  Description of Artificial Sequence: Synthetic oligonucleotide
aggtgtacagcagtggctgg

SEQ ID NO: 284 - DNA, 20 bp, Artificial Sequence
  Description of Artificial Sequence: Synthetic oligonucleotide
ggtgtacagcagtggctggt

SEQ ID NO: 285 - DNA, 20 bp, Artificial Sequence
  Description of Artificial Sequence: Synthetic oligonucleotide
cggatgtggtcagccttgtg

SEQ ID NO: 286 - DNA, 20 bp, Artificial Sequence
  Description of Artificial Sequence: Synthetic oligonucleotide
cactgacagcgtgaacaggt

SEQ ID NO: 287 - DNA, 20 bp, Artificial Sequence
  Description of Artificial Sequence: Synthetic oligonucleotide
actgacagcgtgaacaggta

SEQ ID NO: 288 - DNA, 20 bp, Artificial Sequence
  Description of Artificial Sequence: Synthetic oligonucleotide
gctcactctctgtctgggct

SEQ ID NO: 289 - DNA, 20 bp, Artificial Sequence
  Description of Artificial Sequence: Synthetic oligonucleotide
ctggctgtggagcggactgg

SEQ ID NO: 290 - DNA, 20 bp, Artificial Sequence
  Description of Artificial Sequence: Synthetic oligonucleotide
gctctgactgtacggggcaa

SEQ ID NO: 291 - DNA, 20 bp, Artificial Sequence
  Description of Artificial Sequence: Synthetic oligonucleotide
agctctgactgtacggggca

SEQ ID NO: 292 - DNA, 20 bp, Artificial Sequence
  Description of Artificial Sequence: Synthetic oligonucleotide
acagtaccccttccagctct

SEQ ID NO: 293 - DNA, 20 bp, Artificial Sequence
  Description of Artificial Sequence: Synthetic oligonucleotide
cgtcggccaccagacggttg

SEQ ID NO: 294 - DNA, 20 bp, Artificial Sequence
  Description of Artificial Sequence: Synthetic oligonucleotide
ccagccactgctgtacacct

SEQ ID NO: 295 - DNA, 20 bp, Artificial Sequence
  Description of Artificial Sequence: Synthetic oligonucleotide
accccggccctgcctatgcc

SEQ ID NO: 296 - DNA, 20 bp, Artificial Sequence
  Description of Artificial Sequence: Synthetic oligonucleotide
ggtatcagcagtgcaggagg

SEQ ID NO: 297 - DNA, 20 bp, Artificial Sequence
  Description of Artificial Sequence: Synthetic oligonucleotide
gatgtggtcagccttgtgca

SEQ ID NO: 298 - DNA, 20 bp, Artificial Sequence
  Description of Artificial Sequence: Synthetic oligonucleotide
ggatgtggtcagccttgtgc

SEQ ID NO: 299 - DNA, 20 bp, Artificial Sequence
  Description of Artificial Sequence: Synthetic oligonucleotide
ggccacgcatcctggccttt

SEQ ID NO: 300 - DNA, 20 bp, Artificial Sequence
  Description of Artificial Sequence: Synthetic oligonucleotide
gaaaggccaggatgcgtggc

SEQ ID NO: 301 - DNA, 20 bp, Artificial Sequence
  Description of Artificial Sequence: Synthetic oligonucleotide
actgcttgtccaggccacgc

SEQ ID NO: 302 - DNA, 20 bp, Artificial Sequence
  Description of Artificial Sequence: Synthetic oligonucleotide
tctggactccaactgcttgt

SEQ ID NO: 303 - DNA, 20 bp, Artificial Sequence
  Description of Artificial Sequence: Synthetic oligonucleotide
gtctggactccaactgcttg

SEQ ID NO: 304 - DNA, 20 bp, Artificial Sequence
  Description of Artificial Sequence: Synthetic oligonucleotide
gcttccgtctggactccaac

SEQ ID NO: 305 - DNA, 20 bp, Artificial Sequence
  Description of Artificial Sequence: Synthetic oligonucleotide
gacggaagctggagtcggca

SEQ ID NO: 306 - DNA, 20 bp, Artificial Sequence
  Description of Artificial Sequence: Synthetic oligonucleotide
cgctgtcagtgaaaaccact

SEQ ID NO: 307 - DNA, 20 bp, Artificial Sequence
  Description of Artificial Sequence: Synthetic oligonucleotide
ctgacagcgtgaacaggtag

SEQ ID NO: 308 - DNA, 20 bp, Artificial Sequence
  Description of Artificial Sequence: Synthetic oligonucleotide
ttacggccagcgaggcccga

SEQ ID NO: 309 - DNA, 20 bp, Artificial Sequence
  Description of Artificial Sequence: Synthetic oligonucleotide
attacggccagcgaggcccg

SEQ ID NO: 310 - DNA, 20 bp, Artificial Sequence
  Description of Artificial Sequence: Synthetic oligonucleotide
ggaatctggtgagtctgagg

SEQ ID NO: 311 - DNA, 20 bp, Artificial Sequence
  Description of Artificial Sequence: Synthetic oligonucleotide
ccctcagactcaccagattc

SEQ ID NO: 312 - DNA, 20 bp, Artificial Sequence
  Description of Artificial Sequence: Synthetic oligonucleotide
cgaaggtaggagaaggtctt

SEQ ID NO: 313 - DNA, 20 bp, Artificial Sequence
  Description of Artificial Sequence: Synthetic oligonucleotide
gaaggtaggagaaggtcttg

SEQ ID NO: 314 - DNA, 20 bp, Artificial Sequence
  Description of Artificial Sequence: Synthetic oligonucleotide
gcacctttggctcactctct

SEQ ID NO: 315 - DNA, 20 bp, Artificial Sequence
  Description of Artificial Sequence: Synthetic oligonucleotide
tcgaggaggtggcagagggt

SEQ ID NO: 316 - DNA, 20 bp, Artificial Sequence
  Description of Artificial Sequence: Synthetic oligonucleotide
tggaactgcccaagccagtc

SEQ ID NO: 317 - DNA, 20 bp, Artificial Sequence
  Description of Artificial Sequence: Synthetic oligonucleotide
agggacggggcccacagggg

SEQ ID NO: 318 - DNA, 20 bp, Artificial Sequence
  Description of Artificial Sequence: Synthetic oligonucleotide
gggacggggcccacaggggc

SEQ ID NO: 319 - DNA, 20 bp, Artificial Sequence
  Description of Artificial Sequence: Synthetic oligonucleotide
ctccacagccagcaaaggac

SEQ ID NO: 320 - DNA, 20 bp, Artificial Sequence
  Description of Artificial Sequence: Synthetic oligonucleotide
cagccagcaaaggacgaggt

SEQ ID NO: 321 - DNA, 20 bp, Artificial Sequence
  Description of Artificial Sequence: Synthetic oligonucleotide
ctgccttctagacctcgtcc

SEQ ID NO: 322 - DNA, 20 bp, Artificial Sequence
  Description of Artificial Sequence: Synthetic oligonucleotide
cctaaggaggatgcgcctag

SEQ ID NO: 323 - DNA, 20 bp, Artificial Sequence
  Description of Artificial Sequence: Synthetic oligonucleotide
tggcctcctgcactgctgat

SEQ ID NO: 324 - DNA, 20 bp, Artificial Sequence
  Description of Artificial Sequence: Synthetic oligonucleotide
agcagtgcaggaggccacat

SEQ ID NO: 325 - DNA, 20 bp, Artificial Sequence
  Description of Artificial Sequence: Synthetic oligonucleotide
ccgactccagcttccgtctg

SEQ ID NO: 326 - DNA, 20 bp, Artificial Sequence
  Description of Artificial Sequence: Synthetic oligonucleotide
ggggttccattacggccagc

SEQ ID NO: 327 - DNA, 20 bp, Artificial Sequence
  Description of Artificial Sequence: Synthetic oligonucleotide
cacagcagatcctcctctgg

SEQ ID NO: 328 - DNA, 20 bp, Artificial Sequence
  Description of Artificial Sequence: Synthetic oligonucleotide
attgccccgtacagtcagag

SEQ ID NO: 329 - DNA, 20 bp, Artificial Sequence
  Description of Artificial Sequence: Synthetic oligonucleotide
cccgtacagtcagagctgga

SEQ ID NO: 330 - DNA, 20 bp, Artificial Sequence
  Description of Artificial Sequence: Synthetic oligonucleotide
tggtggaggagcaggcagtg

SEQ ID NO: 331 - DNA, 20 bp, Artificial Sequence
  Description of Artificial Sequence: Synthetic oligonucleotide
tccttaggcataggcagggc

SEQ ID NO: 332 - DNA, 20 bp, Artificial Sequence
  Description of Artificial Sequence: Synthetic oligonucleotide
cggccctgcctatgcctaag

SEQ ID NO: 333 - DNA, 20 bp, Artificial Sequence
  Description of Artificial Sequence: Synthetic oligonucleotide
taggcataggcagggccggg

SEQ ID NO: 334 - DNA, 20 bp, Artificial Sequence
  Description of Artificial Sequence: Synthetic oligonucleotide
aggcagggccggggtgggag

SEQ ID NO: 335 - DNA, 20 bp, Artificial Sequence
  Description of Artificial Sequence: Synthetic oligonucleotide
gcaggatcggggctgtcgct

SEQ ID NO: 336 - DNA, 20 bp, Artificial Sequence
  Description of Artificial Sequence: Synthetic oligonucleotide
ctgcacaaggctgaccacat

SEQ ID NO: 337 - DNA, 20 bp, Artificial Sequence
  Description of Artificial Sequence: Synthetic oligonucleotide
tgcacaaggctgaccacatc

SEQ ID NO: 338 - DNA, 20 bp, Artificial Sequence
  Description of Artificial Sequence: Synthetic oligonucleotide
ctgaccacatccggaaaggc

SEQ ID NO: 339 - DNA, 20 bp, Artificial Sequence
  Description of Artificial Sequence: Synthetic oligonucleotide
ggccacgcatcctggccttt

SEQ ID NO: 340 - DNA, 20 bp, Artificial Sequence
  Description of Artificial Sequence: Synthetic oligonucleotide
gcgtggcctggacaagcagt

SEQ ID NO: 341 - DNA, 20 bp, Artificial Sequence
  Description of Artificial Sequence: Synthetic oligonucleotide
gacaagcagttggagtccag

SEQ ID NO: 342 - DNA, 20 bp, Artificial Sequence
  Description of Artificial Sequence: Synthetic oligonucleotide
gttggagtccagacggaagc

SEQ ID NO: 343 - DNA, 20 bp, Artificial Sequence
  Description of Artificial Sequence: Synthetic oligonucleotide
atgcgtacattggtggggcc

SEQ ID NO: 344 - DNA, 20 bp, Artificial Sequence
  Description of Artificial Sequence: Synthetic oligonucleotide
tggccccaccaatgtacgca

SEQ ID NO: 345 - DNA, 20 bp, Artificial Sequence
  Description of Artificial Sequence: Synthetic oligonucleotide
gctacctgttcacgctgtca

SEQ ID NO: 346 - DNA, 20 bp, Artificial Sequence
  Description of Artificial Sequence: Synthetic oligonucleotide
tgacagcgtgaacaggtagc

SEQ ID NO: 347 - DNA, 20 bp, Artificial Sequence
  Description of Artificial Sequence: Synthetic oligonucleotide
gtcgggcctcgctggccgta

SEQ ID NO: 348 - DNA, 20 bp, Artificial Sequence
  Description of Artificial Sequence: Synthetic oligonucleotide
gcacttgcctaggctggtat

SEQ ID NO: 349 - DNA, 20 bp, Artificial Sequence
  Description of Artificial Sequence: Synthetic oligonucleotide
gggaatctggtgagtctgag

SEQ ID NO: 350 - DNA, 20 bp, Artificial Sequence
  Description of Artificial Sequence: Synthetic oligonucleotide
ctcaccagattcccgaaggt

SEQ ID NO: 351 - DNA, 20 bp, Artificial Sequence
  Description of Artificial Sequence: Synthetic oligonucleotide
ctcctaccttcgggaatctg

SEQ ID NO: 352 - DNA, 20 bp, Artificial Sequence
  Description of Artificial Sequence: Synthetic oligonucleotide
caagaccttctcctaccttc

SEQ ID NO: 353 - DNA, 20 bp, Artificial Sequence
  Description of Artificial Sequence: Synthetic oligonucleotide
ccaagaccttctcctacctt

SEQ ID NO: 354 - DNA, 20 bp, Artificial Sequence
  Description of Artificial Sequence: Synthetic oligonucleotide
gccaagaccttctcctacct

SEQ ID NO: 355 - DNA, 20 bp, Artificial Sequence
  Description of Artificial Sequence: Synthetic oligonucleotide
tatgcacagcagatcctcct

SEQ ID NO: 356 - DNA, 20 bp, Artificial Sequence
  Description of Artificial Sequence: Synthetic oligonucleotide
caaaggtgctggacccagag

SEQ ID NO: 357 - DNA, 20 bp, Artificial Sequence
  Description of Artificial Sequence: Synthetic oligonucleotide
ggctcactctctgtctgggc

SEQ ID NO: 358 - DNA, 20 bp, Artificial Sequence
  Description of Artificial Sequence: Synthetic oligonucleotide
agggtaccccagcccagaca

SEQ ID NO: 359 - DNA, 20 bp, Artificial Sequence
  Description of Artificial Sequence: Synthetic oligonucleotide
agagggtaccccagcccaga

SEQ ID NO: 360 - DNA, 20 bp, Artificial Sequence
  Description of Artificial Sequence: Synthetic oligonucleotide
gtaccctctgccacctcctc

SEQ ID NO: 361 - DNA, 20 bp, Artificial Sequence
  Description of Artificial Sequence: Synthetic oligonucleotide
ccttcctcgaggaggtggca

SEQ ID NO: 362 - DNA, 20 bp, Artificial Sequence
  Description of Artificial Sequence: Synthetic oligonucleotide
atgactggcttgggcagttc

SEQ ID NO: 363 - DNA, 20 bp, Artificial Sequence
  Description of Artificial Sequence: Synthetic oligonucleotide
ggcccctgtgggccccgtcc

SEQ ID NO: 364 - DNA, 20 bp, Artificial Sequence
  Description of Artificial Sequence: Synthetic oligonucleotide
aggacgaggtctagaaggca

SEQ ID NO: 365 - DNA, 20 bp, Artificial Sequence
  Description of Artificial Sequence: Synthetic oligonucleotide
tataagtggaggcgtcgcgc

SEQ ID NO: 366 - DNA, 20 bp, Artificial Sequence
  Description of Artificial Sequence: Synthetic oligonucleotide
gggcacgcgtttaatataag

SEQ ID NO: 367 - DNA, 20 bp, Artificial Sequence
  Description of Artificial Sequence: Synthetic oligonucleotide
actcacgctggatagcctcc

SEQ ID NO: 368 - DNA, 20 bp, Artificial Sequence
  Description of Artificial Sequence: Synthetic oligonucleotide
ggccgagatgtctcgctccg

SEQ ID NO: 369 - DNA, 20 bp, Artificial Sequence
  Description of Artificial Sequence: Synthetic oligonucleotide
cacgcgtttaatataagtgg

SEQ ID NO: 370 - DNA, 20 bp, Artificial Sequence
  Description of Artificial Sequence: Synthetic oligonucleotide
aagtggaggcgtcgcgctgg

SEQ ID NO: 371 - DNA, 20 bp, Artificial Sequence
  Description of Artificial Sequence: Synthetic oligonucleotide
gagtagcgcgagcacagcta

SEQ ID NO: 372 - DNA, 20 bp, Artificial Sequence
  Description of Artificial Sequence: Synthetic oligonucleotide
agtggaggcgtcgcgctggc

SEQ ID NO: 373 - DNA, 20 bp, Artificial Sequence
  Description of Artificial Sequence: Synthetic oligonucleotide
gcccgaatgctgtcagcttc

SEQ ID NO: 374 - DNA, 20 bp, Artificial Sequence
  Description of Artificial Sequence: Synthetic oligonucleotide
cgcgagcacagctaaggcca

SEQ ID NO: 375 - DNA, 20 bp, Artificial Sequence
  Description of Artificial Sequence: Synthetic oligonucleotide
ctcgcgctactctctctttc

SEQ ID NO: 376 - DNA, 20 bp, Artificial Sequence
  Description of Artificial Sequence: Synthetic oligonucleotide
ggccacggagcgagacatct

SEQ ID NO: 377 - DNA, 20 bp, Artificial Sequence
  Description of Artificial Sequence: Synthetic oligonucleotide
cgtgagtaaacctgaatctt

SEQ ID NO: 378 - DNA, 20 bp, Artificial Sequence
  Description of Artificial Sequence: Synthetic oligonucleotide
agtcacatggttcacacggc

SEQ ID NO: 379 - DNA, 20 bp, Artificial Sequence
  Description of Artificial Sequence: Synthetic oligonucleotide
aagtcaacttcaatgtcgga

SEQ ID NO: 380 - DNA, 20 bp, Artificial Sequence
  Description of Artificial Sequence: Synthetic oligonucleotide
cagtaagtcaacttcaatgt

SEQ ID NO: 381 - DNA, 20 bp, Artificial Sequence
  Description of Artificial Sequence: Synthetic oligonucleotide
acccagacacatagcaattc

SEQ ID NO: 382 - DNA, 20 bp, Artificial Sequence
  Description of Artificial Sequence: Synthetic oligonucleotide
gcatactcatctttttcagt

SEQ ID NO: 383 - DNA, 20 bp, Artificial Sequence
  Description of Artificial Sequence: Synthetic oligonucleotide
acagcccaagatagttaagt

SEQ ID NO: 384 - DNA, 20 bp, Artificial Sequence
  Description of Artificial Sequence: Synthetic oligonucleotide
ggcatactcatctttttcag

SEQ ID NO: 385 - DNA, 20 bp, Artificial Sequence
  Description of Artificial Sequence: Synthetic oligonucleotide
ttcctgaagctgacagcatt

SEQ ID NO: 386 - DNA, 20 bp, Artificial Sequence
  Description of Artificial Sequence: Synthetic oligonucleotide
tcacgtcatccagcagagaa

SEQ ID NO: 387 - DNA, 20 bp, Artificial Sequence
  Description of Artificial Sequence: Synthetic oligonucleotide
cagcccaagatagttaagtg

SEQ ID NO: 388 - DNA, 18 bp, Artificial Sequence
  Description of Artificial Sequence: Synthetic oligonucleotide
aattctctctccattctt

SEQ ID NO: 389 - DNA, 19 bp, Artificial Sequence
  Description of Artificial Sequence: Synthetic oligonucleotide
aattctctctccattcttc

SEQ ID NO: 390 - DNA, 20 bp, Artificial Sequence
  Description of Artificial Sequence: Synthetic oligonucleotide
aattctctctccattcttca

SEQ ID NO: 391 - DNA, 21 bp, Artificial Sequence
  Description of Artificial Sequence: Synthetic oligonucleotide
aattctctctccattcttcag

SEQ ID NO: 392 - DNA, 22 bp, Artificial Sequence
  Description of Artificial Sequence: Synthetic oligonucleotide
aattctctctccattcttcagt

SEQ ID NO: 393 - DNA, 23 bp, Artificial Sequence
  Description of Artificial Sequence: Synthetic oligonucleotide
aattctctctccattcttcagta

SEQ ID NO: 394 - DNA, 24 bp, Artificial Sequence
  Description of Artificial Sequence: Synthetic oligonucleotide
aattctctctccattcttcagtaa

SEQ ID NO: 395 - DNA, 18 bp, Artificial Sequence
  Description of Artificial Sequence: Synthetic oligonucleotide
actttccattctctgctg

SEQ ID NO: 396 - DNA, 19 bp, Artificial Sequence
  Description of Artificial Sequence: Synthetic oligonucleotide
actttccattctctgctgg

SEQ ID NO: 397 - DNA, 20 bp, Artificial Sequence
  Description of Artificial Sequence: Synthetic oligonucleotide
actttccattctctgctgga

SEQ ID NO: 398 - DNA, 21 bp, Artificial Sequence
  Description of Artificial Sequence: Synthetic oligonucleotide
actttccattctctgctggat

SEQ ID NO: 399 - DNA, 22 bp, Artificial Sequence
  Description of Artificial Sequence: Synthetic oligonucleotide
actttccattctctgctggatg

SEQ ID NO: 400 - DNA, 23 bp, Artificial Sequence
  Description of Artificial Sequence: Synthetic oligonucleotide
actttccattctctgctggatga

SEQ ID NO: 401 - DNA, 24 bp, Artificial Sequence
  Description of Artificial Sequence: Synthetic oligonucleotide
actttccattctctgctggatgac

SEQ ID NO: 402 - DNA, 18 bp, Artificial Sequence
  Description of Artificial Sequence: Synthetic oligonucleotide
agcaaggactggtctttc

SEQ ID NO: 403 - DNA, 19 bp, Artificial Sequence
  Description of Artificial Sequence: Synthetic oligonucleotide
agcaaggactggtctttct

SEQ ID NO: 404 - DNA, 20 bp, Artificial Sequence
  Description of Artificial Sequence: Synthetic oligonucleotide
agcaaggactggtctttcta

SEQ ID NO: 405 - DNA, 21 bp, Artificial Sequence
  Description of Artificial Sequence: Synthetic oligonucleotide
agcaaggactggtctttctat

SEQ ID NO: 406 - DNA, 22 bp, Artificial Sequence
  Description of Artificial Sequence: Synthetic oligonucleotide
agcaaggactggtctttctatc

SEQ ID NO: 407 - DNA, 23 bp, Artificial Sequence
  Description of Artificial Sequence: Synthetic oligonucleotide
agcaaggactggtctttctatct

SEQ ID NO: 408 - DNA, 24 bp, Artificial Sequence
  Description of Artificial Sequence: Synthetic oligonucleotide
agcaaggactggtctttctatctc

SEQ ID NO: 409 - DNA, 18 bp, Artificial Sequence
  Description of Artificial Sequence: Synthetic oligonucleotide
agtgggggtgaattcagt

SEQ ID NO: 410 - DNA, 19 bp, Artificial Sequence
  Description of Artificial Sequence: Synthetic oligonucleotide
agtgggggtgaattcagtg

SEQ ID NO: 411 - DNA, 20 bp, Artificial Sequence
  Description of Artificial Sequence: Synthetic oligonucleotide
agtgggggtgaattcagtgt

SEQ ID NO: 412 - DNA, 21 bp, Artificial Sequence
  Description of Artificial Sequence: Synthetic oligonucleotide
agtgggggtgaattcagtgta

SEQ ID NO: 413 - DNA, 22 bp, Artificial Sequence
  Description of Artificial Sequence: Synthetic oligonucleotide
agtgggggtgaattcagtgtag

SEQ ID NO: 414 - DNA, 23 bp, Artificial Sequence
  Description of Artificial Sequence: Synthetic oligonucleotide
agtgggggtgaattcagtgtagt

SEQ ID NO: 415 - DNA, 24 bp, Artificial Sequence
  Description of Artificial Sequence: Synthetic oligonucleotide
agtgggggtgaattcagtgtagta

SEQ ID NO: 416 - DNA, 18 bp, Artificial Sequence
  Description of Artificial Sequence: Synthetic oligonucleotide
atccatccgacattgaag

SEQ ID NO: 417 - DNA, 19 bp, Artificial Sequence
  Description of Artificial Sequence: Synthetic oligonucleotide
atccatccgacattgaagt

SEQ ID NO: 418 - DNA, 20 bp, Artificial Sequence
  Description of Artificial Sequence: Synthetic oligonucleotide
atccatccgacattgaagtt

SEQ ID NO: 419 - DNA, 21 bp, Artificial Sequence
  Description of Artificial Sequence: Synthetic oligonucleotide
atccatccgacattgaagttg

SEQ ID NO: 420 - DNA, 22 bp, Artificial Sequence
  Description of Artificial Sequence: Synthetic oligonucleotide
atccatccgacattgaagttga

SEQ ID NO: 421 - DNA, 23 bp, Artificial Sequence
  Description of Artificial Sequence: Synthetic oligonucleotide
atccatccgacattgaagttgac

SEQ ID NO: 422 - DNA, 24 bp, Artificial Sequence
  Description of Artificial Sequence: Synthetic oligonucleotide
atccatccgacattgaagttgact

SEQ ID NO: 423 - DNA, 18 bp, Artificial Sequence
  Description of Artificial Sequence: Synthetic oligonucleotide
caattctctctccattct

SEQ ID NO: 424 - DNA, 19 bp, Artificial Sequence
  Description of Artificial Sequence: Synthetic oligonucleotide
caattctctctccattctt

SEQ ID NO: 425 - DNA, 20 bp, Artificial Sequence
  Description of Artificial Sequence: Synthetic oligonucleotide
caattctctctccattcttc

SEQ ID NO: 426 - DNA, 21 bp, Artificial Sequence
  Description of Artificial Sequence: Synthetic oligonucleotide
caattctctctccattcttca

SEQ ID NO: 427 - DNA, 22 bp, Artificial Sequence
  Description of Artificial Sequence: Synthetic oligonucleotide
caattctctctccattcttcag

SEQ ID NO: 428 - DNA, 23 bp, Artificial Sequence
  Description of Artificial Sequence: Synthetic oligonucleotide
caattctctctccattcttcagt

SEQ ID NO: 429 - DNA, 24 bp, Artificial Sequence
  Description of Artificial Sequence: Synthetic oligonucleotide
caattctctctccattcttcagta

SEQ ID NO: 430 - DNA, 18 bp, Artificial Sequence
  Description of Artificial Sequence: Synthetic oligonucleotide
cagtgggggtgaattcag

SEQ ID NO: 431 - DNA, 19 bp, Artificial Sequence
  Description of Artificial Sequence: Synthetic oligonucleotide
cagtgggggtgaattcagt

SEQ ID NO: 432 - DNA, 20 bp, Artificial Sequence
  Description of Artificial Sequence: Synthetic oligonucleotide
cagtgggggtgaattcagtg

SEQ ID NO: 433 - DNA, 21 bp, Artificial Sequence
  Description of Artificial Sequence: Synthetic oligonucleotide
cagtgggggtgaattcagtgt

SEQ ID NO: 434 - DNA, 22 bp, Artificial Sequence
  Description of Artificial Sequence: Synthetic oligonucleotide
cagtgggggtgaattcagtgta

SEQ ID NO: 435 - DNA, 23 bp, Artificial Sequence
  Description of Artificial Sequence: Synthetic oligonucleotide
cagtgggggtgaattcagtgtag

SEQ ID NO: 436 - DNA, 24 bp, Artificial Sequence
  Description of Artificial Sequence: Synthetic oligonucleotide
cagtgggggtgaattcagtgtagt

SEQ ID NO: 437 - DNA, 18 bp, Artificial Sequence
  Description of Artificial Sequence: Synthetic oligonucleotide
cattctctgctggatgac

SEQ ID NO: 438 - DNA, 19 bp, Artificial Sequence
  Description of Artificial Sequence: Synthetic oligonucleotide
cattctctgctggatgacg

SEQ ID NO: 439 - DNA, 20 bp, Artificial Sequence
  Description of Artificial Sequence: Synthetic oligonucleotide
cattctctgctggatgacgt

SEQ ID NO: 440 - DNA, 21 bp, Artificial Sequence
  Description of Artificial Sequence: Synthetic oligonucleotide
cattctctgctggatgacgtg

SEQ ID NO: 441 - DNA, 22 bp, Artificial Sequence
  Description of Artificial Sequence: Synthetic oligonucleotide
cattctctgctggatgacgtga

SEQ ID NO: 442 - DNA, 23 bp, Artificial Sequence
  Description of Artificial Sequence: Synthetic oligonucleotide
cattctctgctggatgacgtgag

SEQ ID NO: 443 - DNA, 24 bp, Artificial Sequence
  Description of Artificial Sequence: Synthetic oligonucleotide
cattctctgctggatgacgtgagt

SEQ ID NO: 444 - DNA, 18 bp, Artificial Sequence
  Description of Artificial Sequence: Synthetic oligonucleotide
cccgatattcctcaggta

SEQ ID NO: 445 - DNA, 19 bp, Artificial Sequence
  Description of Artificial Sequence: Synthetic oligonucleotide
cccgatattcctcaggtac

SEQ ID NO: 446 - DNA, 20 bp, Artificial Sequence
  Description of Artificial Sequence: Synthetic oligonucleotide
cccgatattcctcaggtact

SEQ ID NO: 447 - DNA, 21 bp, Artificial Sequence
  Description of Artificial Sequence: Synthetic oligonucleotide
cccgatattcctcaggtactc

SEQ ID NO: 448 - DNA, 22 bp, Artificial Sequence
  Description of Artificial Sequence: Synthetic oligonucleotide
cccgatattcctcaggtactcc

SEQ ID NO: 449 - DNA, 23 bp, Artificial Sequence
  Description of Artificial Sequence: Synthetic oligonucleotide
cccgatattcctcaggtactcca

SEQ ID NO: 450 - DNA, 24 bp, Artificial Sequence
  Description of Artificial Sequence: Synthetic oligonucleotide
cccgatattcctcaggtactccaa

SEQ ID NO: 451 - DNA, 18 bp, Artificial Sequence
  Description of Artificial Sequence: Synthetic oligonucleotide
ccgatattcctcaggtac

SEQ ID NO: 452 - DNA, 19 bp, Artificial Sequence
  Description of Artificial Sequence: Synthetic oligonucleotide
ccgatattcctcaggtact

SEQ ID NO: 453 - DNA, 20 bp, Artificial Sequence
  Description of Artificial Sequence: Synthetic oligonucleotide
ccgatattcctcaggtactc

SEQ ID NO: 454 - DNA, 21 bp, Artificial Sequence
  Description of Artificial Sequence: Synthetic oligonucleotide
ccgatattcctcaggtactcc

SEQ ID NO: 455 - DNA, 22 bp, Artificial Sequence
  Description of Artificial Sequence: Synthetic oligonucleotide
ccgatattcctcaggtactcca

SEQ ID NO: 456 - DNA, 23 bp, Artificial Sequence
  Description of Artificial Sequence: Synthetic oligonucleotide
ccgatattcctcaggtactccaa

SEQ ID NO: 457 - DNA, 24 bp, Artificial Sequence
  Description of Artificial Sequence: Synthetic oligonucleotide
ccgatattcctcaggtactccaaa

SEQ ID NO: 458 - DNA, 18 bp, Artificial Sequence
  Description of Artificial Sequence: Synthetic oligonucleotide
ctcacgtcatccagcaga

SEQ ID NO: 459 - DNA, 19 bp, Artificial Sequence
  Description of Artificial Sequence: Synthetic oligonucleotide
ctcacgtcatccagcagag

SEQ ID NO: 460 - DNA, 20 bp, Artificial Sequence
  Description of Artificial Sequence: Synthetic oligonucleotide
ctcacgtcatccagcagaga

SEQ ID NO: 461 - DNA, 21 bp, Artificial Sequence
  Description of Artificial Sequence: Synthetic oligonucleotide
ctcacgtcatccagcagagaa

SEQ ID NO: 462 - DNA, 22 bp, Artificial Sequence
  Description of Artificial Sequence: Synthetic oligonucleotide
ctcacgtcatccagcagagaat

SEQ ID NO: 463 - DNA, 23 bp, Artificial Sequence
  Description of Artificial Sequence: Synthetic oligonucleotide
ctcacgtcatccagcagagaatg

SEQ ID NO: 464 - DNA, 24 bp, Artificial Sequence
  Description of Artificial Sequence: Synthetic oligonucleotide
ctcacgtcatccagcagagaatgg

SEQ ID NO: 465 - DNA, 18 bp, Artificial Sequence
  Description of Artificial Sequence: Synthetic oligonucleotide
ctgaattgctatgtgtct

SEQ ID NO: 466 - DNA, 19 bp, Artificial Sequence
  Description of Artificial Sequence: Synthetic oligonucleotide
ctgaattgctatgtgtctg

SEQ ID NO: 467 - DNA, 20 bp, Artificial Sequence
  Description of Artificial Sequence: Synthetic oligonucleotide
ctgaattgctatgtgtctgg

SEQ ID NO: 468 - DNA, 21 bp, Artificial Sequence
  Description of Artificial Sequence: Synthetic oligonucleotide
ctgaattgctatgtgtctggg

SEQ ID NO: 469 - DNA, 22 bp, Artificial Sequence
  Description of Artificial Sequence: Synthetic oligonucleotide
ctgaattgctatgtgtctgggt

SEQ ID NO: 470 - DNA, 23 bp, Artificial Sequence
  Description of Artificial Sequence: Synthetic oligonucleotide
ctgaattgctatgtgtctgggtt

SEQ ID NO: 471 - DNA, 24 bp, Artificial Sequence
  Description of Artificial Sequence: Synthetic oligonucleotide
ctgaattgctatgtgtctgggttt

SEQ ID NO: 472 - DNA, 18 bp, Artificial Sequence
  Description of Artificial Sequence: Synthetic oligonucleotide
gagtacctgaggaatatc

SEQ ID NO: 473 - DNA, 19 bp, Artificial Sequence
  Description of Artificial Sequence: Synthetic oligonucleotide
gagtacctgaggaatatcg

SEQ ID NO: 474 - DNA, 20 bp, Artificial Sequence
  Description of Artificial Sequence: Synthetic oligonucleotide
gagtacctgaggaatatcgg

SEQ ID NO: 475 - DNA, 21 bp, Artificial Sequence
  Description of Artificial Sequence: Synthetic oligonucleotide
gagtacctgaggaatatcggg

SEQ ID NO: 476 - DNA, 22 bp, Artificial Sequence
  Description of Artificial Sequence: Synthetic oligonucleotide
gagtacctgaggaatatcggga

SEQ ID NO: 477 - DNA, 23 bp, Artificial Sequence
  Description of Artificial Sequence: Synthetic oligonucleotide
gagtacctgaggaatatcgggaa

SEQ ID NO: 478 - DNA, 24 bp, Artificial Sequence
  Description of Artificial Sequence: Synthetic oligonucleotide
gagtacctgaggaatatcgggaaa

SEQ ID NO: 479 - DNA, 18 bp, Artificial Sequence
  Description of Artificial Sequence: Synthetic oligonucleotide
tatctcttgtactacact

SEQ ID NO: 480 - DNA, 19 bp, Artificial Sequence
  Description of Artificial Sequence: Synthetic oligonucleotide
tatctcttgtactacactg

SEQ ID NO: 481 - DNA, 20 bp, Artificial Sequence
  Description of Artificial Sequence: Synthetic oligonucleotide
tatctcttgtactacactga

SEQ ID NO: 482 - DNA, 21 bp, Artificial Sequence
  Description of Artificial Sequence: Synthetic oligonucleotide
tatctcttgtactacactgaa

SEQ ID NO: 483 - DNA, 22 bp, Artificial Sequence
  Description of Artificial Sequence: Synthetic oligonucleotide
tatctcttgtactacactgaat

SEQ ID NO: 484 - DNA, 23 bp, Artificial Sequence
  Description of Artificial Sequence: Synthetic oligonucleotide
tatctcttgtactacactgaatt

SEQ ID NO: 485 - DNA, 24 bp, Artificial Sequence
  Description of Artificial Sequence: Synthetic oligonucleotide
tatctcttgtactacactgaattc

SEQ ID NO: 486 - DNA, 18 bp, Artificial Sequence
  Description of Artificial Sequence: Synthetic oligonucleotide
tcaattctctctccattc

SEQ ID NO: 487 - DNA, 19 bp, Artificial Sequence
  Description of Artificial Sequence: Synthetic oligonucleotide
tcaattctctctccattct

SEQ ID NO: 488 - DNA, 20 bp, Artificial Sequence
  Description of Artificial Sequence: Synthetic oligonucleotide
tcaattctctctccattctt

SEQ ID NO: 489 - DNA, 21 bp, Artificial Sequence
  Description of Artificial Sequence: Synthetic oligonucleotide
tcaattctctctccattcttc

SEQ ID NO: 490 - DNA, 22 bp, Artificial Sequence
  Description of Artificial Sequence: Synthetic oligonucleotide
tcaattctctctccattcttca

SEQ ID NO: 491 - DNA, 23 bp, Artificial Sequence
  Description of Artificial Sequence: Synthetic oligonucleotide
tcaattctctctccattcttcag

SEQ ID NO: 492 - DNA, 24 bp, Artificial Sequence
  Description of Artificial Sequence: Synthetic oligonucleotide
tcaattctctctccattcttcagt

SEQ ID NO: 493 - DNA, 18 bp, Artificial Sequence
  Description of Artificial Sequence: Synthetic oligonucleotide
tcacagcccaagatagtt

SEQ ID NO: 494 - DNA, 19 bp, Artificial Sequence
  Description of Artificial Sequence: Synthetic oligonucleotide
tcacagcccaagatagtta

SEQ ID NO: 495 - DNA, 20 bp, Artificial Sequence
  Description of Artificial Sequence: Synthetic oligonucleotide
tcacagcccaagatagttaa

SEQ ID NO: 496 - DNA, 21 bp, Artificial Sequence
  Description of Artificial Sequence: Synthetic oligonucleotide
tcacagcccaagatagttaag

SEQ ID NO: 497 - DNA, 22 bp, Artificial Sequence
  Description of Artificial Sequence: Synthetic oligonucleotide
tcacagcccaagatagttaagt

SEQ ID NO: 498 - DNA, 23 bp, Artificial Sequence
  Description of Artificial Sequence: Synthetic oligonucleotide
tcacagcccaagatagttaagtg

SEQ ID NO: 499 - DNA, 24 bp, Artificial Sequence
  Description of Artificial Sequence: Synthetic oligonucleotide
tcacagcccaagatagttaagtgg

SEQ ID NO: 500 - DNA, 18 bp, Artificial Sequence
  Description of Artificial Sequence: Synthetic oligonucleotide
tcagtgggggtgaattca

SEQ ID NO: 501 - DNA, 19 bp, Artificial Sequence
  Description of Artificial Sequence: Synthetic oligonucleotide
tcagtgggggtgaattcag

SEQ ID NO: 502 - DNA, 20 bp, Artificial Sequence
  Description of Artificial Sequence: Synthetic oligonucleotide
tcagtgggggtgaattcagt

SEQ ID NO: 503 - DNA, 21 bp, Artificial Sequence
  Description of Artificial Sequence: Synthetic oligonucleotide
tcagtgggggtgaattcagtg

SEQ ID NO: 504 - DNA, 22 bp, Artificial Sequence
  Description of Artificial Sequence: Synthetic oligonucleotide
tcagtgggggtgaattcagtgt

SEQ ID NO: 505 - DNA, 23 bp, Artificial Sequence
  Description of Artificial Sequence: Synthetic oligonucleotide
tcagtgggggtgaattcagtgta

SEQ ID NO: 506 - DNA, 24 bp, Artificial Sequence
  Description of Artificial Sequence: Synthetic oligonucleotide
tcagtgggggtgaattcagtgtag

SEQ ID NO: 507 - DNA, 18 bp, Artificial Sequence
  Description of Artificial Sequence: Synthetic oligonucleotide
tggcctggaggctatcca

SEQ ID NO: 508 - DNA, 19 bp, Artificial Sequence
  Description of Artificial Sequence: Synthetic oligonucleotide
tggcctggaggctatccag

SEQ ID NO: 509 - DNA, 20 bp, Artificial Sequence
  Description of Artificial Sequence: Synthetic oligonucleotide
tggcctggaggctatccagc

SEQ ID NO: 510 - DNA, 21 bp, Artificial Sequence
  Description of Artificial Sequence: Synthetic oligonucleotide
tggcctggaggctatccagcg

SEQ ID NO: 511 - DNA, 22 bp, Artificial Sequence
  Description of Artificial Sequence: Synthetic oligonucleotide
tggcctggaggctatccagcgt

SEQ ID NO: 512 - DNA, 23 bp, Artificial Sequence
  Description of Artificial Sequence: Synthetic oligonucleotide
tggcctggaggctatccagcgtg

SEQ ID NO: 513 - DNA, 24 bp, Artificial Sequence
  Description of Artificial Sequence: Synthetic oligonucleotide
tggcctggaggctatccagcgtga

SEQ ID NO: 514 - DNA, 18 bp, Artificial Sequence
  Description of Artificial Sequence: Synthetic oligonucleotide
atagatcgagacatgtaa

SEQ ID NO: 515 - DNA, 19 bp, Artificial Sequence
  Description of Artificial Sequence: Synthetic oligonucleotide
atagatcgagacatgtaag

SEQ ID NO: 516 - DNA, 20 bp, Artificial Sequence
  Description of Artificial Sequence: Synthetic oligonucleotide
atagatcgagacatgtaagc

SEQ ID NO: 517 - DNA, 21 bp, Artificial Sequence
  Description of Artificial Sequence: Synthetic oligonucleotide
atagatcgagacatgtaagca

SEQ ID NO: 518 - DNA, 22 bp, Artificial Sequence
  Description of Artificial Sequence: Synthetic oligonucleotide
atagatcgagacatgtaagcag

SEQ ID NO: 519 - DNA, 23 bp, Artificial Sequence
  Description of Artificial Sequence: Synthetic oligonucleotide
atagatcgagacatgtaagcagc

SEQ ID NO: 520 - DNA, 24 bp, Artificial Sequence
  Description of Artificial Sequence: Synthetic oligonucleotide
atagatcgagacatgtaagcagca

SEQ ID NO: 521 - DNA, 18 bp, Artificial Sequence
  Description of Artificial Sequence: Synthetic oligonucleotide
catagatcgagacatgta

SEQ ID NO: 522 - DNA, 19 bp, Artificial Sequence
  Description of Artificial Sequence: Synthetic oligonucleotide
catagatcgagacatgtaa

SEQ ID NO: 523 - DNA, 20 bp, Artificial Sequence
  Description of Artificial Sequence: Synthetic oligonucleotide
catagatcgagacatgtaag

SEQ ID NO: 524 - DNA, 21 bp, Artificial Sequence
  Description of Artificial Sequence: Synthetic oligonucleotide
catagatcgagacatgtaagc

SEQ ID NO: 525 - DNA, 22 bp, Artificial Sequence
  Description of Artificial Sequence: Synthetic oligonucleotide
catagatcgagacatgtaagca

SEQ ID NO: 526 - DNA, 23 bp, Artificial Sequence
  Description of Artificial Sequence: Synthetic oligonucleotide
catagatcgagacatgtaagcag

SEQ ID NO: 527 - DNA, 24 bp, Artificial Sequence
  Description of Artificial Sequence: Synthetic oligonucleotide
catagatcgagacatgtaagcagc

SEQ ID NO: 528 - DNA, 18 bp, Artificial Sequence
  Description of Artificial Sequence: Synthetic oligonucleotide
ctccactgtctttttcat

SEQ ID NO: 529 - DNA, 19 bp, Artificial Sequence
  Description of Artificial Sequence: Synthetic oligonucleotide
ctccactgtctttttcata

SEQ ID NO: 530 - DNA, 20 bp, Artificial Sequence
  Description of Artificial Sequence: Synthetic oligonucleotide
ctccactgtctttttcatag

SEQ ID NO: 531 - DNA, 21 bp, Artificial Sequence
  Description of Artificial Sequence: Synthetic oligonucleotide
ctccactgtctttttcataga

SEQ ID NO: 532 - DNA, 22 bp, Artificial Sequence
  Description of Artificial Sequence: Synthetic oligonucleotide
ctccactgtctttttcatagat

SEQ ID NO: 533 - DNA, 23 bp, Artificial Sequence
  Description of Artificial Sequence: Synthetic oligonucleotide
ctccactgtctttttcatagatc

SEQ ID NO: 534 - DNA, 24 bp, Artificial Sequence
  Description of Artificial Sequence: Synthetic oligonucleotide
ctccactgtctttttcatagatcg

SEQ ID NO: 535 - DNA, 18 bp, Artificial Sequence
  Description of Artificial Sequence: Synthetic oligonucleotide
tcatagatcgagacatgt

SEQ ID NO: 536 - DNA, 19 bp, Artificial Sequence
  Description of Artificial Sequence: Synthetic oligonucleotide
tcatagatcgagacatgta

SEQ ID NO: 537 - DNA, 20 bp, Artificial Sequence
  Description of Artificial Sequence: Synthetic oligonucleotide
tcatagatcgagacatgtaa

SEQ ID NO: 538 - DNA, 21 bp, Artificial Sequence
  Description of Artificial Sequence: Synthetic oligonucleotide
tcatagatcgagacatgtaag

SEQ ID NO: 539 - DNA, 22 bp, Artificial Sequence
  Description of Artificial Sequence: Synthetic oligonucleotide
tcatagatcgagacatgtaagc

SEQ ID NO: 540 - DNA, 23 bp, Artificial Sequence
  Description of Artificial Sequence: Synthetic oligonucleotide
tcatagatcgagacatgtaagca

SEQ ID NO: 541 - DNA, 24 bp, Artificial Sequence
  Description of Artificial Sequence: Synthetic oligonucleotide
tcatagatcgagacatgtaagcag

SEQ ID NO: 542 - DNA, 18 bp, Artificial Sequence
  Description of Artificial Sequence: Synthetic oligonucleotide
tccactgtctttttcata

SEQ ID NO: 543 - DNA, 19 bp, Artificial Sequence
  Description of Artificial Sequence: Synthetic oligonucleotide
tccactgtctttttcatag

SEQ ID NO: 544 - DNA, 20 bp, Artificial Sequence
  Description of Artificial Sequence: Synthetic oligonucleotide
tccactgtctttttcataga

SEQ ID NO: 545 - DNA, 21 bp, Artificial Sequence
  Description of Artificial Sequence: Synthetic oligonucleotide
tccactgtctttttcatagat

SEQ ID NO: 546 - DNA, 22 bp, Artificial Sequence
  Description of Artificial Sequence: Synthetic oligonucleotide
tccactgtctttttcatagatc

SEQ ID NO: 547 - DNA, 23 bp, Artificial Sequence
  Description of Artificial Sequence: Synthetic oligonucleotide
tccactgtctttttcatagatcg

SEQ ID NO: 548 - DNA, 24 bp, Artificial Sequence
  Description of Artificial Sequence: Synthetic oligonucleotide
tccactgtctttttcatagatcga

SEQ ID NO: 549 - DNA, 18 bp, Artificial Sequence
  Description of Artificial Sequence: Synthetic oligonucleotide
tctccactgtctttttca

SEQ ID NO: 550 - DNA, 19 bp, Artificial Sequence
  Description of Artificial Sequence: Synthetic oligonucleotide
tctccactgtctttttcat

SEQ ID NO: 551 - DNA, 20 bp, Artificial Sequence
  Description of Artificial Sequence: Synthetic oligonucleotide
tctccactgtctttttcata

SEQ ID NO: 552 - DNA, 21 bp, Artificial Sequence
  Description of Artificial Sequence: Synthetic oligonucleotide
tctccactgtctttttcatag

SEQ ID NO: 553 - DNA, 22 bp, Artificial Sequence
  Description of Artificial Sequence: Synthetic oligonucleotide
tctccactgtctttttcataga

SEQ ID NO: 554 - DNA, 23 bp, Artificial Sequence
  Description of Artificial Sequence: Synthetic oligonucleotide
tctccactgtctttttcatagat

SEQ ID NO: 555 - DNA, 24 bp, Artificial Sequence
  Description of Artificial Sequence: Synthetic oligonucleotide
tctccactgtctttttcatagatc

SEQ ID NO: 556 - DNA, 18 bp, Artificial Sequence
  Description of Artificial Sequence: Synthetic oligonucleotide
ttctccactgtctttttc

SEQ ID NO: 557 - DNA, 19 bp, Artificial Sequence
  Description of Artificial Sequence: Synthetic oligonucleotide
ttctccactgtctttttca

SEQ ID NO: 558 - DNA, 20 bp, Artificial Sequence
  Description of Artificial Sequence: Synthetic oligonucleotide
ttctccactgtctttttcat

SEQ ID NO: 559 - DNA, 21 bp, Artificial Sequence
  Description of Artificial Sequence: Synthetic oligonucleotide
ttctccactgtctttttcata

SEQ ID NO: 560 - DNA, 22 bp, Artificial Sequence
  Description of Artificial Sequence: Synthetic oligonucleotide
ttctccactgtctttttcatag

SEQ ID NO: 561 - DNA, 23 bp, Artificial Sequence
  Description of Artificial Sequence: Synthetic oligonucleotide
ttctccactgtctttttcataga

SEQ ID NO: 562 - DNA, 24 bp, Artificial Sequence
  Description of Artificial Sequence: Synthetic oligonucleotide
ttctccactgtctttttcatagat

SEQ ID NO: 563 - DNA, 18 bp, Artificial Sequence
  Description of Artificial Sequence: Synthetic oligonucleotide
tttctccactgtcttttt

SEQ ID NO: 564 - DNA, 19 bp, Artificial Sequence
  Description of Artificial Sequence: Synthetic oligonucleotide
tttctccactgtctttttc

SEQ ID NO: 565 - DNA, 20 bp, Artificial Sequence
  Description of Artificial Sequence: Synthetic oligonucleotide
tttctccactgtctttttca

SEQ ID NO: 566 - DNA, 21 bp, Artificial Sequence
  Description of Artificial Sequence: Synthetic oligonucleotide
tttctccactgtctttttcat

SEQ ID NO: 567 - DNA, 22 bp, Artificial Sequence
  Description of Artificial Sequence: Synthetic oligonucleotide
tttctccactgtctttttcata

SEQ ID NO: 568 - DNA, 23 bp, Artificial Sequence
  Description of Artificial Sequence: Synthetic oligonucleotide
tttctccactgtctttttcatag

SEQ ID NO: 569 - DNA, 24 bp, Artificial Sequence
  Description of Artificial Sequence: Synthetic oligonucleotide
tttctccactgtctttttcataga

SEQ ID NO: 570 - DNA, 18 bp, Artificial Sequence
  Description of Artificial Sequence: Synthetic oligonucleotide
ttttctccactgtctttt

SEQ ID NO: 571 - DNA, 19 bp, Artificial Sequence
  Description of Artificial Sequence: Synthetic oligonucleotide
ttttctccactgtcttttt

SEQ ID NO: 572 - DNA, 20 bp, Artificial Sequence
  Description of Artificial Sequence: Synthetic oligonucleotide
ttttctccactgtctttttc

SEQ ID NO: 573 - DNA, 21 bp, Artificial Sequence
  Description of Artificial Sequence: Synthetic oligonucleotide
ttttctccactgtctttttca

SEQ ID NO: 574 - DNA, 22 bp, Artificial Sequence
  Description of Artificial Sequence: Synthetic oligonucleotide
ttttctccactgtctttttcat

SEQ ID NO: 575 - DNA, 23 bp, Artificial Sequence
  Description of Artificial Sequence: Synthetic oligonucleotide
ttttctccactgtctttttcata

SEQ ID NO: 576 - DNA, 24 bp, Artificial Sequence
  Description of Artificial Sequence: Synthetic oligonucleotide
ttttctccactgtctttttcatag

SEQ ID NO: 577 - DNA, 21 bp, Artificial Sequence
  Description of Artificial Sequence: Synthetic oligonucleotide
gaggtaaagcgtttgcatttg

SEQ ID NO: 578 - DNA, 21 bp, Artificial Sequence
  Description of Artificial Sequence: Synthetic oligonucleotide
cctctaaagcttatgcttaca

SEQ ID NO: 579 - DNA, 21 bp, Artificial Sequence
  Description of Artificial Sequence: Synthetic oligonucleotide
agtcgatttacttgtagcact

SEQ ID NO: 580 - DNA, 21 bp, Artificial Sequence
  Description of Artificial Sequence: Synthetic oligonucleotide
cttgtagcactgcacagttaa

SEQ ID NO: 581 - DNA, 21 bp, Artificial Sequence
  Description of Artificial Sequence: Synthetic oligonucleotide
tccattacaggataaaagact

SEQ ID NO: 582 - DNA, 21 bp, Artificial Sequence
  Description of Artificial Sequence: Synthetic oligonucleotide
ctccattacaggataaaagac

SEQ ID NO: 583 - DNA, 21 bp, Artificial Sequence
  Description of Artificial Sequence: Synthetic oligonucleotide
tctccattacaggataaaaga

SEQ ID NO: 584 - DNA, 21 bp, Artificial Sequence
  Description of Artificial Sequence: Synthetic oligonucleotide
atcctgtaatggagaaaaatc

SEQ ID NO: 585 - DNA, 21 bp, Artificial Sequence
  Description of Artificial Sequence: Synthetic oligonucleotide
tcctgtaatggagaaaaatcc

SEQ ID NO: 586 - DNA, 21 bp, Artificial Sequence
  Description of Artificial Sequence: Synthetic oligonucleotide
aaacatgagtaagttgttttg

SEQ ID NO: 587 - DNA, 21 bp, Artificial Sequence
  Description of Artificial Sequence: Synthetic oligonucleotide
gctttcaaacatgagtaagtt

SEQ ID NO: 588 - DNA, 21 bp, Artificial Sequence
  Description of Artificial Sequence: Synthetic oligonucleotide
aaagccaaaccattcattgtc

SEQ ID NO: 589 - DNA, 21 bp, Artificial Sequence
  Description of Artificial Sequence: Synthetic oligonucleotide
gtaacagcagtcatcatccat

SEQ ID NO: 590 - DNA, 21 bp, Artificial Sequence
  Description of Artificial Sequence: Synthetic oligonucleotide
accatcctcatggattggtgt

SEQ ID NO: 591 - DNA, 21 bp, Artificial Sequence
  Description of Artificial Sequence: Synthetic oligonucleotide
tgtccatcatttcaccatcct

SEQ ID NO: 592 - DNA, 21 bp, Artificial Sequence
  Description of Artificial Sequence: Synthetic oligonucleotide
gaaatttctgtccatcatttc

SEQ ID NO: 593 - DNA, 21 bp, Artificial Sequence
  Description of Artificial Sequence: Synthetic oligonucleotide
agaaatttctgtccatcattt

SEQ ID NO: 594 - DNA, 21 bp, Artificial Sequence
  Description of Artificial Sequence: Synthetic oligonucleotide
ttttagaaatttctgtccatc

SEQ ID NO: 595 - DNA, 21 bp, Artificial Sequence
  Description of Artificial Sequence: Synthetic oligonucleotide
cttttagaaatttctgtccat

SEQ ID NO: 596 - DNA, 21 bp, Artificial Sequence
  Description of Artificial Sequence: Synthetic oligonucleotide
ttttcttttagaaatttctgt

SEQ ID NO: 597 - DNA, 21 bp, Artificial Sequence
  Description of Artificial Sequence: Synthetic oligonucleotide
taaaagaaaagaaagaatttt

SEQ ID NO: 598 - DNA, 21 bp, Artificial Sequence
  Description of Artificial Sequence: Synthetic oligonucleotide
aaacatttacatcttaccatt

SEQ ID NO: 599 - DNA, 21 bp, Artificial Sequence
  Description of Artificial Sequence: Synthetic oligonucleotide
catcttaccatttcttcttca

SEQ ID NO: 600 - DNA, 21 bp, Artificial Sequence
  Description of Artificial Sequence: Synthetic oligonucleotide
tatagataatgaagaagaaat

SEQ ID NO: 601 - DNA, 21 bp, Artificial Sequence
  Description of Artificial Sequence: Synthetic oligonucleotide
ttcttcattatctatagaaag

SEQ ID NO: 602 - DNA, 21 bp, Artificial Sequence
  Description of Artificial Sequence: Synthetic oligonucleotide
ctggcctgtacttcgaagaac

SEQ ID NO: 603 - DNA, 21 bp, Artificial Sequence
  Description of Artificial Sequence: Synthetic oligonucleotide
cttaccaatgtagtaacaact

SEQ ID NO: 604 - DNA, 21 bp, Artificial Sequence
  Description of Artificial Sequence: Synthetic oligonucleotide
gcacgtcattgtggccattgt

SEQ ID NO: 605 - DNA, 21 bp, Artificial Sequence
  Description of Artificial Sequence: Synthetic oligonucleotide
tttagcacgtcattgtggcca

SEQ ID NO: 606 - DNA, 21 bp, Artificial Sequence
  Description of Artificial Sequence: Synthetic oligonucleotide
ccatcagctccagagaagctc

SEQ ID NO: 607 - DNA, 21 bp, Artificial Sequence
  Description of Artificial Sequence: Synthetic oligonucleotide
tctccctgcagatttaccatc

SEQ ID NO: 608 - DNA, 21 bp, Artificial Sequence
  Description of Artificial Sequence: Synthetic oligonucleotide
aaatgctttacctttgcagtg

SEQ ID NO: 609 - DNA, 21 bp, Artificial Sequence
  Description of Artificial Sequence: Synthetic oligonucleotide
aatgctttacctttgcagtga

SEQ ID NO: 610 - DNA, 21 bp, Artificial Sequence
  Description of Artificial Sequence: Synthetic oligonucleotide
cctttgcagtgataggttttg

SEQ ID NO: 611 - DNA, 21 bp, Artificial Sequence
  Description of Artificial Sequence: Synthetic oligonucleotide
cagtgataggttttgtcattc

SEQ ID NO: 612 - DNA, 21 bp, Artificial Sequence
  Description of Artificial Sequence: Synthetic oligonucleotide
aagggaatgacaaaacctatc

SEQ ID NO: 613 - DNA, 21 bp, Artificial Sequence
  Description of Artificial Sequence: Synthetic oligonucleotide
caagggaatgacaaaacctat

SEQ ID NO: 614 - DNA, 21 bp, Artificial Sequence
  Description of Artificial Sequence: Synthetic oligonucleotide
gtcattcccttgaaaatcctg

SEQ ID NO: 615 - DNA, 21 bp, Artificial Sequence
  Description of Artificial Sequence: Synthetic oligonucleotide
tcattcccttgaaaatcctga

SEQ ID NO: 616 - DNA, 21 bp, Artificial Sequence
  Description of Artificial Sequence: Synthetic oligonucleotide
tgaaggtttaattccgcatag

SEQ ID NO: 617 - DNA, 21 bp, Artificial Sequence
  Description of Artificial Sequence: Synthetic oligonucleotide
gaaggtttaattccgcatagg

SEQ ID NO: 618 - DNA, 21 bp, Artificial Sequence
  Description of Artificial Sequence: Synthetic oligonucleotide
aaggtttaattccgcataggt

SEQ ID NO: 619 - DNA, 21 bp, Artificial Sequence
  Description of Artificial Sequence: Synthetic oligonucleotide
attccgcataggttatttcct

SEQ ID NO: 620 - DNA, 21 bp, Artificial Sequence
  Description of Artificial Sequence: Synthetic oligonucleotide
gcaactgaacaggaaataacc

SEQ ID NO: 621 - DNA, 21 bp, Artificial Sequence
  Description of Artificial Sequence: Synthetic oligonucleotide
agcaactgaacaggaaataac

SEQ ID NO: 622 - DNA, 21 bp, Artificial Sequence
  Description of Artificial Sequence: Synthetic oligonucleotide
ctgttcagttgctaaaatgga

SEQ ID NO: 623 - DNA, 21 bp, Artificial Sequence
  Description of Artificial Sequence: Synthetic oligonucleotide
tattgcctttaggttttcgtt

SEQ ID NO: 624 - DNA, 21 bp, Artificial Sequence
  Description of Artificial Sequence: Synthetic oligonucleotide
attgcctttaggttttcgttg

SEQ ID NO: 625 - DNA, 21 bp, Artificial Sequence
  Description of Artificial Sequence: Synthetic oligonucleotide
ttgcctttaggttttcgttgc

SEQ ID NO: 626 - DNA, 21 bp, Artificial Sequence
  Description of Artificial Sequence: Synthetic oligonucleotide
ggttttcgttgctgcctcttt

SEQ ID NO: 627 - DNA, 21 bp, Artificial Sequence
  Description of Artificial Sequence: Synthetic oligonucleotide
cgttgctgcctctttgggttt

SEQ ID NO: 628 - DNA, 21 bp, Artificial Sequence
  Description of Artificial Sequence: Synthetic oligonucleotide
gttgctgcctctttgggtttg

SEQ ID NO: 629 - DNA, 21 bp, Artificial Sequence
  Description of Artificial Sequence: Synthetic oligonucleotide
ggtttgggggcagattcaggt

SEQ ID NO: 630 - DNA, 21 bp, Artificial Sequence
  Description of Artificial Sequence: Synthetic oligonucleotide
ggggcagattcaggtctgagt

SEQ ID NO: 631 - DNA, 20 bp, Artificial Sequence
  Description of Artificial Sequence: Synthetic oligonucleotide
tctgcagaaatgttccccgt

SEQ ID NO: 632 - DNA, 20 bp, Artificial Sequence
  Description of Artificial Sequence: Synthetic oligonucleotide
tgcagagaaaggtggctcta

SEQ ID NO: 633 - DNA, 20 bp, Artificial Sequence
  Description of Artificial Sequence: Synthetic oligonucleotide
taatgctgacttggggtggc

SEQ ID NO: 634 - DNA, 20 bp, Artificial Sequence
  Description of Artificial Sequence: Synthetic oligonucleotide
taggacctccaggaagattc

SEQ ID NO: 635 - DNA, 20 bp, Artificial Sequence
  Description of Artificial Sequence: Synthetic oligonucleotide
tagtcaacgcgaccaccacg

SEQ ID NO: 636 - DNA, 20 bp, Artificial Sequence
  Description of Artificial Sequence: Synthetic oligonucleotide
tcctgaggtcaccttccaca

SEQ ID NO: 637 - DNA, 20 bp, Artificial Sequence
  Description of Artificial Sequence: Synthetic oligonucleotide
tattgtgcctgtcatcattc

SEQ ID NO: 638 - DNA, 21 bp, Artificial Sequence
  Description of Artificial Sequence: Synthetic oligonucleotide
tgacaggcacaatagaaacaa

SEQ ID NO: 639 - DNA, 21 bp, Artificial Sequence
  Description of Artificial Sequence: Synthetic oligonucleotide
gacaggcacaatagaaacaac

SEQ ID NO: 640 - DNA, 21 bp, Artificial Sequence
  Description of Artificial Sequence: Synthetic oligonucleotide
aaacaacggggaacatttctg

SEQ ID NO: 641 - DNA, 21 bp, Artificial Sequence
  Description of Artificial Sequence: Synthetic oligonucleotide
acaacggggaacatttctgca

SEQ ID NO: 642 - DNA, 21 bp, Artificial Sequence
  Description of Artificial Sequence: Synthetic oligonucleotide
tgatagagccacctttctctg

SEQ ID NO: 643 - DNA, 21 bp, Artificial Sequence
  Description of Artificial Sequence: Synthetic oligonucleotide
gggtcacttgtgccgtggtgg

SEQ ID NO: 644 - DNA, 21 bp, Artificial Sequence
  Description of Artificial Sequence: Synthetic oligonucleotide
ggcacaagtgacccaggtcaa

SEQ ID NO: 645 - DNA, 21 bp, Artificial Sequence
  Description of Artificial Sequence: Synthetic oligonucleotide
gtcctgctgctcccagttgac

SEQ ID NO: 646 - DNA, 21 bp, Artificial Sequence
  Description of Artificial Sequence: Synthetic oligonucleotide
tggccatttgtaatgctgact

SEQ ID NO: 647 - DNA, 21 bp, Artificial Sequence
  Description of Artificial Sequence: Synthetic oligonucleotide
tggcacatctccccatccttc

SEQ ID NO: 648 - DNA, 21 bp, Artificial Sequence
  Description of Artificial Sequence: Synthetic oligonucleotide
catctccccatccttcaagga

SEQ ID NO: 649 - DNA, 21 bp, Artificial Sequence
  Description of Artificial Sequence: Synthetic oligonucleotide
ccactcgatccttgaaggatg

SEQ ID NO: 650 - DNA, 21 bp, Artificial Sequence
  Description of Artificial Sequence: Synthetic oligonucleotide
ggccactcgatccttgaagga

SEQ ID NO: 651 - DNA, 21 bp, Artificial Sequence
  Description of Artificial Sequence: Synthetic oligonucleotide
cctggggccactcgatccttg

SEQ ID NO: 652 - DNA, 21 bp, Artificial Sequence
  Description of Artificial Sequence: Synthetic oligonucleotide
gactggagggtgaggcccagg

SEQ ID NO: 653 - DNA, 21 bp, Artificial Sequence
  Description of Artificial Sequence: Synthetic oligonucleotide
atcgttcacggtcagcgactg

SEQ ID NO: 654 - DNA, 21 bp, Artificial Sequence
  Description of Artificial Sequence: Synthetic oligonucleotide
gtcgctgaccgtgaacgatac

SEQ ID NO: 655 - DNA, 21 bp, Artificial Sequence
  Description of Artificial Sequence: Synthetic oligonucleotide
cgctgaccgtgaacgatacag

SEQ ID NO: 656 - DNA, 21 bp, Artificial Sequence
  Description of Artificial Sequence: Synthetic oligonucleotide
gcatctatcacacctaccctg

SEQ ID NO: 657 - DNA, 21 bp, Artificial Sequence
  Description of Artificial Sequence: Synthetic oligonucleotide
cctaccctgatgggacgtaca

SEQ ID NO: 658 - DNA, 21 bp, Artificial Sequence
  Description of Artificial Sequence: Synthetic oligonucleotide
taccctgatgggacgtacact

SEQ ID NO: 659 - DNA, 21 bp, Artificial Sequence
  Description of Artificial Sequence: Synthetic oligonucleotide
ccctgatgggacgtacactgg

SEQ ID NO: 660 - DNA, 21 bp, Artificial Sequence
  Description of Artificial Sequence: Synthetic oligonucleotide
ttctcccagtgtacgtcccat

SEQ ID NO: 661 - DNA, 21 bp, Artificial Sequence
  Description of Artificial Sequence: Synthetic oligonucleotide
ggagaatcttcctggaggtcc

SEQ ID NO: 662 - DNA, 21 bp, Artificial Sequence
  Description of Artificial Sequence: Synthetic oligonucleotide
catggctccaagcaatggaat

SEQ ID NO: 663 - DNA, 21 bp, Artificial Sequence
  Description of Artificial Sequence: Synthetic oligonucleotide
cgcggccatggctccaagcaa

SEQ ID NO: 664 - DNA, 21 bp, Artificial Sequence
  Description of Artificial Sequence: Synthetic oligonucleotide
tcgcggccatggctccaagca

SEQ ID NO: 665 - DNA, 21 bp, Artificial Sequence
  Description of Artificial Sequence: Synthetic oligonucleotide
catcgtggtggtcgcgttgac

SEQ ID NO: 666 - DNA, 21 bp, Artificial Sequence
  Description of Artificial Sequence: Synthetic oligonucleotide
aaagccctcagaatccattct

SEQ ID NO: 667 - DNA, 21 bp, Artificial Sequence
  Description of Artificial Sequence: Synthetic oligonucleotide
cattctgtggaaggtgacctc

SEQ ID NO: 668 - DNA, 21 bp, Artificial Sequence
  Description of Artificial Sequence: Synthetic oligonucleotide
ttctgtggaaggtgacctcag

SEQ ID NO: 669 - DNA, 21 bp, Artificial Sequence
  Description of Artificial Sequence: Synthetic oligonucleotide
cctgaggtcaccttccacaga

SEQ ID NO: 670 - DNA, 21 bp, Artificial Sequence
  Description of Artificial Sequence: Synthetic oligonucleotide
ttctcctgaggtcaccttcca

SEQ ID NO: 671 - DNA, 21 bp, Artificial Sequence
  Description of Artificial Sequence: Synthetic oligonucleotide
aggagaaaatcagctggacag

SEQ ID NO: 672 - DNA, 21 bp, Artificial Sequence
  Description of Artificial Sequence: Synthetic oligonucleotide
ggagaaaatcagctggacagg

SEQ ID NO: 673 - DNA, 21 bp, Artificial Sequence
  Description of Artificial Sequence: Synthetic oligonucleotide
gccccagtgctccctcacccc

SEQ ID NO: 674 - DNA, 21 bp, Artificial Sequence
  Description of Artificial Sequence: Synthetic oligonucleotide
tggacacagcttcctgggggt

SEQ ID NO: 675 - DNA, 21 bp, Artificial Sequence
  Description of Artificial Sequence: Synthetic oligonucleotide
tctgcctggacacagcttcct

SEQ ID NO: 676 - DNA, 21 bp, Artificial Sequence
  Description of Artificial Sequence: Synthetic oligonucleotide
agctgcacctgctgggctctg

SEQ ID NO: 677 - DNA, 21 bp, Artificial Sequence
  Description of Artificial Sequence: Synthetic oligonucleotide
gctgggctctgtggagagcag

SEQ ID NO: 678 - DNA, 21 bp, Artificial Sequence
  Description of Artificial Sequence: Synthetic oligonucleotide
tgggctctgtggagagcagcg

SEQ ID NO: 679 - DNA, 21 bp, Artificial Sequence
  Description of Artificial Sequence: Synthetic oligonucleotide
ctgcatgactacttcaatgtc

SEQ ID NO: 680 - DNA, 21 bp, Artificial Sequence
  Description of Artificial Sequence: Synthetic oligonucleotide
aatgtcctgagttacagaagc

SEQ ID NO: 681 - DNA, 21 bp, Artificial Sequence
  Description of Artificial Sequence: Synthetic oligonucleotide
tgggtaactgcagcttcttca

SEQ ID NO: 682 - DNA, 20 bp, Artificial Sequence
  Description of Artificial Sequence: Synthetic oligonucleotide
gacaggcacaatagaaacaa

SEQ ID NO: 683 - DNA, 20 bp, Artificial Sequence
  Description of Artificial Sequence: Synthetic oligonucleotide
acaggcacaatagaaacaac

SEQ ID NO: 684 - DNA, 20 bp, Artificial Sequence
  Description of Artificial Sequence: Synthetic oligonucleotide
caggcacaatagaaacaacg

SEQ ID NO: 685 - DNA, 20 bp, Artificial Sequence
  Description of Artificial Sequence: Synthetic oligonucleotide
gggaacatttctgcagagaa

SEQ ID NO: 686 - DNA, 20 bp, Artificial Sequence
  Description of Artificial Sequence: Synthetic oligonucleotide
aacatttctgcagagaaagg

SEQ ID NO: 687 - DNA, 20 bp, Artificial Sequence
  Description of Artificial Sequence: Synthetic oligonucleotide
atgtcacctctcctccacca

SEQ ID NO: 688 - DNA, 20 bp, Artificial Sequence
  Description of Artificial Sequence: Synthetic oligonucleotide
cttgtgccgtggtggaggag

SEQ ID NO: 689 - DNA, 20 bp, Artificial Sequence
  Description of Artificial Sequence: Synthetic oligonucleotide
ggtcacttgtgccgtggtgg

SEQ ID NO: 690 - DNA, 20 bp, Artificial Sequence
  Description of Artificial Sequence: Synthetic oligonucleotide
caccacggcacaagtgaccc

SEQ ID NO: 691 - DNA, 20 bp, Artificial Sequence
  Description of Artificial Sequence: Synthetic oligonucleotide
ctgggtcacttgtgccgtgg

SEQ ID NO: 692 - DNA, 20 bp, Artificial Sequence
  Description of Artificial Sequence: Synthetic oligonucleotide
gacctgggtcacttgtgccg

SEQ ID NO: 693 - DNA, 20 bp, Artificial Sequence
  Description of Artificial Sequence: Synthetic oligonucleotide
cacaagtgacccaggtcaac

SEQ ID NO: 694 - DNA, 20 bp, Artificial Sequence
  Description of Artificial Sequence: Synthetic oligonucleotide
acaagtgacccaggtcaact

SEQ ID NO: 695 - DNA, 20 bp, Artificial Sequence
  Description of Artificial Sequence: Synthetic oligonucleotide
ccaggtcaactgggagcagc

SEQ ID NO: 696 - DNA, 20 bp, Artificial Sequence
  Description of Artificial Sequence: Synthetic oligonucleotide
ctgctgctcccagttgacct

SEQ ID NO: 697 - DNA, 20 bp, Artificial Sequence
  Description of Artificial Sequence: Synthetic oligonucleotide
cctgctgctcccagttgacc

SEQ ID NO: 698 - DNA, 20 bp, Artificial Sequence
  Description of Artificial Sequence: Synthetic oligonucleotide
ggagcagcaggaccagcttc

SEQ ID NO: 699 - DNA, 20 bp, Artificial Sequence
  Description of Artificial Sequence: Synthetic oligonucleotide
cattacaaatggccagaagc

SEQ ID NO: 700 - DNA, 20 bp, Artificial Sequence
  Description of Artificial Sequence: Synthetic oligonucleotide
ggccatttgtaatgctgact

SEQ ID NO: 701 - DNA, 20 bp, Artificial Sequence
  Description of Artificial Sequence: Synthetic oligonucleotide
gccatttgtaatgctgactt

SEQ ID NO: 702 - DNA, 20 bp, Artificial Sequence
  Description of Artificial Sequence: Synthetic oligonucleotide
ccatttgtaatgctgacttg

SEQ ID NO: 703 - DNA, 20 bp, Artificial Sequence
  Description of Artificial Sequence: Synthetic oligonucleotide
tttgtaatgctgacttgggg

SEQ ID NO: 704 - DNA, 20 bp, Artificial Sequence
  Description of Artificial Sequence: Synthetic oligonucleotide
ccccaagtcagcattacaaa

SEQ ID NO: 705 - DNA, 20 bp, Artificial Sequence
  Description of Artificial Sequence: Synthetic oligonucleotide
gcacatctccccatccttca

SEQ ID NO: 706 - DNA, 20 bp, Artificial Sequence
  Description of Artificial Sequence: Synthetic oligonucleotide
cccatccttcaaggatcgag

SEQ ID NO: 707 - DNA, 20 bp, Artificial Sequence
  Description of Artificial Sequence: Synthetic oligonucleotide
cactcgatccttgaaggatg

SEQ ID NO: 708 - DNA, 20 bp, Artificial Sequence
  Description of Artificial Sequence: Synthetic oligonucleotide
ccactcgatccttgaaggat

SEQ ID NO: 709 - DNA, 20 bp, Artificial Sequence
  Description of Artificial Sequence: Synthetic oligonucleotide
gccactcgatccttgaagga

SEQ ID NO: 710 - DNA, 20 bp, Artificial Sequence
  Description of Artificial Sequence: Synthetic oligonucleotide
ttcaaggatcgagtggcccc

SEQ ID NO: 711 - DNA, 20 bp, Artificial Sequence
  Description of Artificial Sequence: Synthetic oligonucleotide
tggggccactcgatccttga

SEQ ID NO: 712 - DNA, 20 bp, Artificial Sequence
  Description of Artificial Sequence: Synthetic oligonucleotide
gatcgagtggccccaggtcc

SEQ ID NO: 713 - DNA, 20 bp, Artificial Sequence
  Description of Artificial Sequence: Synthetic oligonucleotide
agtggccccaggtcccggcc

SEQ ID NO: 714 - DNA, 20 bp, Artificial Sequence
  Description of Artificial Sequence: Synthetic oligonucleotide
gtggccccaggtcccggcct

SEQ ID NO: 715 - DNA, 20 bp, Artificial Sequence
  Description of Artificial Sequence: Synthetic oligonucleotide
gaggcccaggccgggacctg

SEQ ID NO: 716 - DNA, 20 bp, Artificial Sequence
  Description of Artificial Sequence: Synthetic oligonucleotide
tgaggcccaggccgggacct

SEQ ID NO: 717 - DNA, 20 bp, Artificial Sequence
  Description of Artificial Sequence: Synthetic oligonucleotide
gtgaggcccaggccgggacc

SEQ ID NO: 718 - DNA, 20 bp, Artificial Sequence
  Description of Artificial Sequence: Synthetic oligonucleotide
tggagggtgaggcccaggcc

SEQ ID NO: 719 - DNA, 20 bp, Artificial Sequence
  Description of Artificial Sequence: Synthetic oligonucleotide
ctggagggtgaggcccaggc

SEQ ID NO: 720 - DNA, 20 bp, Artificial Sequence
  Description of Artificial Sequence: Synthetic oligonucleotide
gcgactggagggtgaggccc

SEQ ID NO: 721 - DNA, 20 bp, Artificial Sequence
  Description of Artificial Sequence: Synthetic oligonucleotide
cggtcagcgactggagggtg

SEQ ID NO: 722 - DNA, 20 bp, Artificial Sequence
  Description of Artificial Sequence: Synthetic oligonucleotide
gttcacggtcagcgactgga

SEQ ID NO: 723 - DNA, 20 bp, Artificial Sequence
  Description of Artificial Sequence: Synthetic oligonucleotide
cgttcacggtcagcgactgg

SEQ ID NO: 724 - DNA, 20 bp, Artificial Sequence
  Description of Artificial Sequence: Synthetic oligonucleotide
tatcgttcacggtcagcgac

SEQ ID NO: 725 - DNA, 20 bp, Artificial Sequence
  Description of Artificial Sequence: Synthetic oligonucleotide
tcgctgaccgtgaacgatac

SEQ ID NO: 726 - DNA, 20 bp, Artificial Sequence
  Description of Artificial Sequence: Synthetic oligonucleotide
cgctgaccgtgaacgataca

SEQ ID NO: 727 - DNA, 20 bp, Artificial Sequence
  Description of Artificial Sequence: Synthetic oligonucleotide
gctgaccgtgaacgatacag

SEQ ID NO: 728 - DNA, 20 bp, Artificial Sequence
  Description of Artificial Sequence: Synthetic oligonucleotide
gtactcccctgtatcgttca

SEQ ID NO: 729 - DNA, 20 bp, Artificial Sequence
  Description of Artificial Sequence: Synthetic oligonucleotide
atctatcacacctaccctga

SEQ ID NO: 730 - DNA, 20 bp, Artificial Sequence
  Description of Artificial Sequence: Synthetic oligonucleotide
tctatcacacctaccctgat

SEQ ID NO: 731 - DNA, 20 bp, Artificial Sequence
  Description of Artificial Sequence: Synthetic oligonucleotide
taccctgatgggacgtacac

SEQ ID NO: 732 - DNA, 20 bp, Artificial Sequence
  Description of Artificial Sequence: Synthetic oligonucleotide
accctgatgggacgtacact

SEQ ID NO: 733 - DNA, 20 bp, Artificial Sequence
  Description of Artificial Sequence: Synthetic oligonucleotide
agtgtacgtcccatcagggt

SEQ ID NO: 734 - DNA, 20 bp, Artificial Sequence
  Description of Artificial Sequence: Synthetic oligonucleotide
tcccagtgtacgtcccatca

SEQ ID NO: 735 - DNA, 20 bp, Artificial Sequence
  Description of Artificial Sequence: Synthetic oligonucleotide
ctcccagtgtacgtcccatc

SEQ ID NO: 736 - DNA, 20 bp, Artificial Sequence
  Description of Artificial Sequence: Synthetic oligonucleotide
gtacactgggagaatcttcc

SEQ ID NO: 737 - DNA, 20 bp, Artificial Sequence
  Description of Artificial Sequence: Synthetic oligonucleotide
cactgggagaatcttcctgg

SEQ ID NO: 738 - DNA, 20 bp, Artificial Sequence
  Description of Artificial Sequence: Synthetic oligonucleotide
ctgagctttctaggacctcc

SEQ ID NO: 739 - DNA, 20 bp, Artificial Sequence
  Description of Artificial Sequence: Synthetic oligonucleotide
aggttccagattccattgct

SEQ ID NO: 740 - DNA, 20 bp, Artificial Sequence
  Description of Artificial Sequence: Synthetic oligonucleotide
aagcaatggaatctggaacc

SEQ ID NO: 741 - DNA, 20 bp, Artificial Sequence
  Description of Artificial Sequence: Synthetic oligonucleotide
gattccattgcttggagcca

SEQ ID NO: 742 - DNA, 20 bp, Artificial Sequence
  Description of Artificial Sequence: Synthetic oligonucleotide
tggctccaagcaatggaatc

SEQ ID NO: 743 - DNA, 20 bp, Artificial Sequence
  Description of Artificial Sequence: Synthetic oligonucleotide
gcggccatggctccaagcaa

SEQ ID NO: 744 - DNA, 20 bp, Artificial Sequence
  Description of Artificial Sequence: Synthetic oligonucleotide
tggagccatggccgcgacgc

SEQ ID NO: 745 - DNA, 20 bp, Artificial Sequence
  Description of Artificial Sequence: Synthetic oligonucleotide
agccatggccgcgacgctgg

SEQ ID NO: 746 - DNA, 20 bp, Artificial Sequence
  Description of Artificial Sequence: Synthetic oligonucleotide
gaccaccagcgtcgcggcca

SEQ ID NO: 747 - DNA, 20 bp, Artificial Sequence
  Description of Artificial Sequence: Synthetic oligonucleotide
gcagatgaccaccagcgtcg

SEQ ID NO: 748 - DNA, 20 bp, Artificial Sequence
  Description of Artificial Sequence: Synthetic oligonucleotide
catctgcacagcagtcatcg

SEQ ID NO: 749 - DNA, 20 bp, Artificial Sequence
  Description of Artificial Sequence: Synthetic oligonucleotide
ctgcacagcagtcatcgtgg

SEQ ID NO: 750 - DNA, 20 bp, Artificial Sequence
  Description of Artificial Sequence: Synthetic oligonucleotide
agccctcagaatccattctg

SEQ ID NO: 751 - DNA, 20 bp, Artificial Sequence
  Description of Artificial Sequence: Synthetic oligonucleotide
ctcagaatccattctgtgga

SEQ ID NO: 752 - DNA, 20 bp, Artificial Sequence
  Description of Artificial Sequence: Synthetic oligonucleotide
ttccacagaatggattctga

SEQ ID NO: 753 - DNA, 20 bp, Artificial Sequence
  Description of Artificial Sequence: Synthetic oligonucleotide
cttccacagaatggattctg

SEQ ID NO: 754 - DNA, 20 bp, Artificial Sequence
  Description of Artificial Sequence: Synthetic oligonucleotide
attctgtggaaggtgacctc

SEQ ID NO: 755 - DNA, 20 bp, Artificial Sequence
  Description of Artificial Sequence: Synthetic oligonucleotide
tgaggtcaccttccacagaa

SEQ ID NO: 756 - DNA, 20 bp, Artificial Sequence
  Description of Artificial Sequence: Synthetic oligonucleotide
gacctcaggagaaaatcagc

SEQ ID NO: 757 - DNA, 20 bp, Artificial Sequence
  Description of Artificial Sequence: Synthetic oligonucleotide
caggagaaaatcagctggac

SEQ ID NO: 758 - DNA, 20 bp, Artificial Sequence
  Description of Artificial Sequence: Synthetic oligonucleotide
gtccagctgattttctcctg

SEQ ID NO: 759 - DNA, 20 bp, Artificial Sequence
  Description of Artificial Sequence: Synthetic oligonucleotide
gagaaaatcagctggacagg

SEQ ID NO: 760 - DNA, 20 bp, Artificial Sequence
  Description of Artificial Sequence: Synthetic oligonucleotide
aatcagctggacaggaggaa

SEQ ID NO: 761 - DNA, 20 bp, Artificial Sequence
  Description of Artificial Sequence: Synthetic oligonucleotide
cccagtgctccctcaccccc

SEQ ID NO: 762 - DNA, 20 bp, Artificial Sequence
  Description of Artificial Sequence: Synthetic oligonucleotide
ctgggggtgagggagcactg

SEQ ID NO: 763 - DNA, 20 bp, Artificial Sequence
  Description of Artificial Sequence: Synthetic oligonucleotide
cctgggggtgagggagcact

SEQ ID NO: 764 - DNA, 20 bp, Artificial Sequence
  Description of Artificial Sequence: Synthetic oligonucleotide
tcctgggggtgagggagcac

SEQ ID NO: 765 - DNA, 20 bp, Artificial Sequence
  Description of Artificial Sequence: Synthetic oligonucleotide
acacagcttcctgggggtga

SEQ ID NO: 766 - DNA, 20 bp, Artificial Sequence
  Description of Artificial Sequence: Synthetic oligonucleotide
gacacagcttcctgggggtg

SEQ ID NO: 767 - DNA, 20 bp, Artificial Sequence
  Description of Artificial Sequence: Synthetic oligonucleotide
acccccaggaagctgtgtcc

SEQ ID NO: 768 - DNA, 20 bp, Artificial Sequence
  Description of Artificial Sequence: Synthetic oligonucleotide
gcctggacacagcttcctgg

SEQ ID NO: 769 - DNA, 20 bp, Artificial Sequence
  Description of Artificial Sequence: Synthetic oligonucleotide
tgcctggacacagcttcctg

SEQ ID NO: 770 - DNA, 20 bp, Artificial Sequence
  Description of Artificial Sequence: Synthetic oligonucleotide
ctgcctggacacagcttcct

SEQ ID NO: 771 - DNA, 20 bp, Artificial Sequence
  Description of Artificial Sequence: Synthetic oligonucleotide
tctgcctggacacagcttcc

SEQ ID NO: 772 - DNA, 20 bp, Artificial Sequence
  Description of Artificial Sequence: Synthetic oligonucleotide
caggcagaagctgcacctgc

SEQ ID NO: 773 - DNA, 20 bp, Artificial Sequence
  Description of Artificial Sequence: Synthetic oligonucleotide
aggcagaagctgcacctgct

SEQ ID NO: 774 - DNA, 20 bp, Artificial Sequence
  Description of Artificial Sequence: Synthetic oligonucleotide
cagcaggtgcagcttctgcc

SEQ ID NO: 775 - DNA, 20 bp, Artificial Sequence
  Description of Artificial Sequence: Synthetic oligonucleotide
gctgcacctgctgggctctg

SEQ ID NO: 776 - DNA, 20 bp, Artificial Sequence
  Description of Artificial Sequence: Synthetic oligonucleotide
tgctctccacagagcccagc

SEQ ID NO: 777 - DNA, 20 bp, Artificial Sequence
  Description of Artificial Sequence: Synthetic oligonucleotide
ctgggctctgtggagagcag

SEQ ID NO: 778 - DNA, 20 bp, Artificial Sequence
  Description of Artificial Sequence: Synthetic oligonucleotide
tgggctctgtggagagcagc

SEQ ID NO: 779 - DNA, 20 bp, Artificial Sequence
  Description of Artificial Sequence: Synthetic oligonucleotide
gggctctgtggagagcagcg

SEQ ID NO: 780 - DNA, 20 bp, Artificial Sequence
  Description of Artificial Sequence: Synthetic oligonucleotide
ctgtggagagcagcggggag

SEQ ID NO: 781 - DNA, 20 bp, Artificial Sequence
  Description of Artificial Sequence: Synthetic oligonucleotide
attgaagtagtcatgcagct

SEQ ID NO: 782 - DNA, 20 bp, Artificial Sequence
  Description of Artificial Sequence: Synthetic oligonucleotide
tgtcctgagttacagaagcc

SEQ ID NO: 783 - DNA, 20 bp, Artificial Sequence
  Description of Artificial Sequence: Synthetic oligonucleotide
gtcctgagttacagaagcct

SEQ ID NO: 784 - DNA, 20 bp, Artificial Sequence
  Description of Artificial Sequence: Synthetic oligonucleotide
tacccaggcttctgtaactc

SEQ ID NO: 785 - DNA, 20 bp, Artificial Sequence
  Description of Artificial Sequence: Synthetic oligonucleotide
tgaagaagctgcagttaccc

SEQ ID NO: 786 - DNA, 20 bp, Artificial Sequence
  Description of Artificial Sequence: Synthetic oligonucleotide
tgcagcttcttcacagagac

SEQ ID NO: 787 - DNA, 20 bp, Artificial Sequence
  Description of Artificial Sequence: Synthetic oligonucleotide
gttgtttctattgtgcctgt

SEQ ID NO: 788 - DNA, 20 bp, Artificial Sequence
  Description of Artificial Sequence: Synthetic oligonucleotide
cgttgtttctattgtgcctg

SEQ ID NO: 789 - DNA, 20 bp, Artificial Sequence
  Description of Artificial Sequence: Synthetic oligonucleotide
ccgttgtttctattgtgcct

SEQ ID NO: 790 - DNA, 20 bp, Artificial Sequence
  Description of Artificial Sequence: Synthetic oligonucleotide
ccacggcacaagtgacccag

SEQ ID NO: 791 - DNA, 20 bp, Artificial Sequence
  Description of Artificial Sequence: Synthetic oligonucleotide
agttgacctgggtcacttgt

SEQ ID NO: 792 - DNA, 20 bp, Artificial Sequence
  Description of Artificial Sequence: Synthetic oligonucleotide
aagtcagcattacaaatggc

SEQ ID NO: 793 - DNA, 20 bp, Artificial Sequence
  Description of Artificial Sequence: Synthetic oligonucleotide
catccttcaaggatcgagtg

SEQ ID NO: 794 - DNA, 20 bp, Artificial Sequence
  Description of Artificial Sequence: Synthetic oligonucleotide
atccttcaaggatcgagtgg

SEQ ID NO: 795 - DNA, 20 bp, Artificial Sequence
  Description of Artificial Sequence: Synthetic oligonucleotide
aggatcgagtggccccaggt

SEQ ID NO: 796 - DNA, 20 bp, Artificial Sequence
  Description of Artificial Sequence: Synthetic oligonucleotide
aggtcccggcctgggcctca

SEQ ID NO: 797 - DNA, 20 bp, Artificial Sequence
  Description of Artificial Sequence: Synthetic oligonucleotide
ggcctgggcctcaccctcca

SEQ ID NO: 798 - DNA, 20 bp, Artificial Sequence
  Description of Artificial Sequence: Synthetic oligonucleotide
cggtcagcgactggagggtg

SEQ ID NO: 799 - DNA, 20 bp, Artificial Sequence
  Description of Artificial Sequence: Synthetic oligonucleotide
gtcgctgaccgtgaacgata

SEQ ID NO: 800 - DNA, 20 bp, Artificial Sequence
  Description of Artificial Sequence: Synthetic oligonucleotide
tgtatcgttcacggtcagcg

SEQ ID NO: 801 - DNA, 20 bp, Artificial Sequence
  Description of Artificial Sequence: Synthetic oligonucleotide
ctgtatcgttcacggtcagc

SEQ ID NO: 802 - DNA, 20 bp, Artificial Sequence
  Description of Artificial Sequence: Synthetic oligonucleotide
atcagggtaggtgtgataga

SEQ ID NO: 803 - DNA, 20 bp, Artificial Sequence
  Description of Artificial Sequence: Synthetic oligonucleotide
agtgtacgtcccatcagggt

SEQ ID NO: 804 - DNA, 20 bp, Artificial Sequence
  Description of Artificial Sequence: Synthetic oligonucleotide
ggaagattctcccagtgtac

SEQ ID NO: 805 - DNA, 20 bp, Artificial Sequence
  Description of Artificial Sequence: Synthetic oligonucleotide
tggaggtcctagaaagctca

SEQ ID NO: 806 - DNA, 20 bp, Artificial Sequence
  Description of Artificial Sequence: Synthetic oligonucleotide
agcaatggaatctggaacct

SEQ ID NO: 807 - DNA, 20 bp, Artificial Sequence
  Description of Artificial Sequence: Synthetic oligonucleotide
agattccattgcttggagcc

SEQ ID NO: 808 - DNA, 20 bp, Artificial Sequence
  Description of Artificial Sequence: Synthetic oligonucleotide
gattccattgcttggagcca

SEQ ID NO: 809 - DNA, 20 bp, Artificial Sequence
  Description of Artificial Sequence: Synthetic oligonucleotide
attgcttggagccatggccg

SEQ ID NO: 810 - DNA, 20 bp, Artificial Sequence
  Description of Artificial Sequence: Synthetic oligonucleotide
ttgcttggagccatggccgc

SEQ ID NO: 811 - DNA, 20 bp, Artificial Sequence
  Description of Artificial Sequence: Synthetic oligonucleotide
cagaatggattctgagggct

SEQ ID NO: 812 - DNA, 20 bp, Artificial Sequence
  Description of Artificial Sequence: Synthetic oligonucleotide
acagaatggattctgagggc

SEQ ID NO: 813 - DNA, 20 bp, Artificial Sequence
  Description of Artificial Sequence: Synthetic oligonucleotide
ttctgtggaaggtgacctca

SEQ ID NO: 814 - DNA, 20 bp, Artificial Sequence
  Description of Artificial Sequence: Synthetic oligonucleotide
gctgattttctcctgaggtc

SEQ ID NO: 815 - DNA, 20 bp, Artificial Sequence
  Description of Artificial Sequence: Synthetic oligonucleotide
tcctgtccagctgattttct

SEQ ID NO: 816 - DNA, 20 bp, Artificial Sequence
  Description of Artificial Sequence: Synthetic oligonucleotide
ttcctcctgtccagctgatt

SEQ ID NO: 817 - DNA, 20 bp, Artificial Sequence
  Description of Artificial Sequence: Synthetic oligonucleotide
tgggggtgagggagcactgg

SEQ ID NO: 818 - DNA, 20 bp, Artificial Sequence
  Description of Artificial Sequence: Synthetic oligonucleotide
agtgctccctcacccccagg

SEQ ID NO: 819 - DNA, 20 bp, Artificial Sequence
  Description of Artificial Sequence: Synthetic oligonucleotide
tcacccccaggaagctgtgt

SEQ ID NO: 820 - DNA, 20 bp, Artificial Sequence
  Description of Artificial Sequence: Synthetic oligonucleotide
caggaagctgtgtccaggca

SEQ ID NO: 821 - DNA, 20 bp, Artificial Sequence
  Description of Artificial Sequence: Synthetic oligonucleotide
aggaagctgtgtccaggcag

SEQ ID NO: 822 - DNA, 20 bp, Artificial Sequence
  Description of Artificial Sequence: Synthetic oligonucleotide
ggcagaagctgcacctgctg

SEQ ID NO: 823 - DNA, 20 bp, Artificial Sequence
  Description of Artificial Sequence: Synthetic oligonucleotide
cagagcccagcaggtgcagc

SEQ ID NO: 824 - DNA, 20 bp, Artificial Sequence
  Description of Artificial Sequence: Synthetic oligonucleotide
gctgctctccacagagccca

SEQ ID NO: 825 - DNA, 20 bp, Artificial Sequence
  Description of Artificial Sequence: Synthetic oligonucleotide
cgctgctctccacagagccc

SEQ ID NO: 826 - DNA, 20 bp, Artificial Sequence
  Description of Artificial Sequence: Synthetic oligonucleotide
atgtcctgagttacagaagc

SEQ ID NO: 827 - DNA, 20 bp, Artificial Sequence
  Description of Artificial Sequence: Synthetic oligonucleotide
gagcacacccactgcgatgt

SEQ ID NO: 828 - DNA, 20 bp, Artificial Sequence
  Description of Artificial Sequence: Synthetic oligonucleotide
gatggccaggagactgaaga

SEQ ID NO: 829 - DNA, 20 bp, Artificial Sequence
  Description of Artificial Sequence: Synthetic oligonucleotide
ctgctcaccggagcgggatg

SEQ ID NO: 830 - DNA, 20 bp, Artificial Sequence
  Description of Artificial Sequence: Synthetic oligonucleotide
gtctgtggccatgcccatca

SEQ ID NO: 831 - DNA, 20 bp, Artificial Sequence
  Description of Artificial Sequence: Synthetic oligonucleotide
tcaccggagcgggatgcgga

SEQ ID NO: 832 - DNA, 20 bp, Artificial Sequence
  Description of Artificial Sequence: Synthetic oligonucleotide
gtggcaggcagcgcagaacc

SEQ ID NO: 833 - DNA, 20 bp, Artificial Sequence
  Description of Artificial Sequence: Synthetic oligonucleotide
agcacaccagcacattgccc

SEQ ID NO: 834 - DNA, 20 bp, Artificial Sequence
  Description of Artificial Sequence: Synthetic oligonucleotide
caggttgctgttgagccaca

SEQ ID NO: 835 - DNA, 20 bp, Artificial Sequence
  Description of Artificial Sequence: Synthetic oligonucleotide
cttcattgcctgcttcgtcc

SEQ ID NO: 836 - DNA, 20 bp, Artificial Sequence
  Description of Artificial Sequence: Synthetic oligonucleotide
gtacaccgaggagcccatga

SEQ ID NO: 837 - DNA, 20 bp, Artificial Sequence
  Description of Artificial Sequence: Synthetic oligonucleotide
gatggcaatgtagcggtcaa

SEQ ID NO: 838 - DNA, 20 bp, Artificial Sequence
  Description of Artificial Sequence: Synthetic oligonucleotide
ctcctcggtgtacatcacgg

SEQ ID NO: 839 - DNA, 20 bp, Artificial Sequence
  Description of Artificial Sequence: Synthetic oligonucleotide
cgaggagcccatgatgggca

SEQ ID NO: 840 - DNA, 20 bp, Artificial Sequence
  Description of Artificial Sequence: Synthetic oligonucleotide
gggctcctcggtgtacatca

SEQ ID NO: 841 - DNA, 20 bp, Artificial Sequence
  Description of Artificial Sequence: Synthetic oligonucleotide
ctttgtggtgtcactggcgg

SEQ ID NO: 842 - DNA, 20 bp, Artificial Sequence
  Description of Artificial Sequence: Synthetic oligonucleotide
ccgctccggtgagcagggcc

SEQ ID NO: 843 - DNA, 20 bp, Artificial Sequence
  Description of Artificial Sequence: Synthetic oligonucleotide
gggttctgcgctgcctgcca

SEQ ID NO: 844 - DNA, 20 bp, Artificial Sequence
  Description of Artificial Sequence: Synthetic oligonucleotide
ggacgaagcaggcaatgaag

SEQ ID NO: 845 - DNA, 20 bp, Artificial Sequence
  Description of Artificial Sequence: Synthetic oligonucleotide
gtgctgatggtgatggcaaa

SEQ ID NO: 846 - DNA, 20 bp, Artificial Sequence
  Description of Artificial Sequence: Synthetic oligonucleotide
agcgcagaacccggtgctga

SEQ ID NO: 847 - DNA, 20 bp, Artificial Sequence
  Description of Artificial Sequence: Synthetic oligonucleotide
gagctccatcttcagtctcc

SEQ ID NO: 848 - DNA, 20 bp, Artificial Sequence
  Description of Artificial Sequence: Synthetic oligonucleotide
tgctgatggtgatggcaaag

SEQ ID NO: 849 - DNA, 20 bp, Artificial Sequence
  Description of Artificial Sequence: Synthetic oligonucleotide
ggcggcggccgacatcgcag

SEQ ID NO: 850 - DNA, 20 bp, Artificial Sequence
  Description of Artificial Sequence: Synthetic oligonucleotide
aatgaagaggcagccgtggc

SEQ ID NO: 851 - DNA, 20 bp, Artificial Sequence
  Description of Artificial Sequence: Synthetic oligonucleotide
gggcaatgtgctggtgtgct

SEQ ID NO: 852 - DNA, 20 bp, Artificial Sequence
  Description of Artificial Sequence: Synthetic oligonucleotide
catgcccatcatgggctcct

SEQ ID NO: 853 - DNA, 20 bp, Artificial Sequence
  Description of Artificial Sequence: Synthetic oligonucleotide
aatgtagcggtcaatggcga

SEQ ID NO: 854 - DNA, 20 bp, Artificial Sequence
  Description of Artificial Sequence: Synthetic oligonucleotide
agtagttggtgacgttctgc

SEQ ID NO: 855 - DNA, 20 bp, Artificial Sequence
  Description of Artificial Sequence: Synthetic oligonucleotide
agcggtcaatggcgatggcc

SEQ ID NO: 856 - DNA, 20 bp, Artificial Sequence
  Description of Artificial Sequence: Synthetic oligonucleotide
cgcatcccgctccggtgagc

SEQ ID NO: 857 - DNA, 20 bp, Artificial Sequence
  Description of Artificial Sequence: Synthetic oligonucleotide
gcatcccgctccggtgagca

SEQ ID NO: 858 - DNA, 20 bp, Artificial Sequence
  Description of Artificial Sequence: Synthetic oligonucleotide
tgggcaatgtgctggtgtgc

SEQ ID NO: 859 - DNA, 20 bp, Artificial Sequence
  Description of Artificial Sequence: Synthetic oligonucleotide
caactactttgtggtgtcac

SEQ ID NO: 860 - DNA, 20 bp, Artificial Sequence
  Description of Artificial Sequence: Synthetic oligonucleotide
cgctccggtgagcagggccg

SEQ ID NO: 861 - DNA, 20 bp, Artificial Sequence
  Description of Artificial Sequence: Synthetic oligonucleotide
gatggtgatggcaaagggga

SEQ ID NO: 862 - DNA, 20 bp, Artificial Sequence
  Description of Artificial Sequence: Synthetic oligonucleotide
ggtgtacatcacggtggagc

SEQ ID NO: 863 - DNA, 20 bp, Artificial Sequence
  Description of Artificial Sequence: Synthetic oligonucleotide
gaacgtcaccaactactttg

SEQ ID NO: 864 - DNA, 20 bp, Artificial Sequence
  Description of Artificial Sequence: Synthetic oligonucleotide
cagtgacaccacaaagtagt

SEQ ID NO: 865 - DNA, 20 bp, Artificial Sequence
  Description of Artificial Sequence: Synthetic oligonucleotide
ggccatcctgggcaatgtgc

SEQ ID NO: 866 - DNA, 20 bp, Artificial Sequence
  Description of Artificial Sequence: Synthetic oligonucleotide
cccggccctgctcaccggag

SEQ ID NO: 867 - DNA, 20 bp, Artificial Sequence
  Description of Artificial Sequence: Synthetic oligonucleotide
caccagcacattgcccagga

SEQ ID NO: 868 - DNA, 20 bp, Artificial Sequence
  Description of Artificial Sequence: Synthetic oligonucleotide
tttgccatcaccatcagcac

SEQ ID NO: 869 - DNA, 20 bp, Artificial Sequence
  Description of Artificial Sequence: Synthetic oligonucleotide
ctccaccgtgatgtacaccg

SEQ ID NO: 870 - DNA, 20 bp, Artificial Sequence
  Description of Artificial Sequence: Synthetic oligonucleotide
ggagctggccattgctgtgc

SEQ ID NO: 871 - DNA, 20 bp, Artificial Sequence
  Description of Artificial Sequence: Synthetic oligonucleotide
caggatggccagcacagcaa

SEQ ID NO: 872 - DNA, 20 bp, Artificial Sequence
  Description of Artificial Sequence: Synthetic oligonucleotide
gaacccggtgctgatggtga

SEQ ID NO: 873 - DNA, 20 bp, Artificial Sequence
  Description of Artificial Sequence: Synthetic oligonucleotide
tggagctctgcgtgaggacc

SEQ ID NO: 874 - DNA, 20 bp, Artificial Sequence
  Description of Artificial Sequence: Synthetic oligonucleotide
cccgctccggtgagcagggc

SEQ ID NO: 875 - DNA, 20 bp, Artificial Sequence
  Description of Artificial Sequence: Synthetic oligonucleotide
aggcaatgaagaggcagccg

SEQ ID NO: 876 - DNA, 20 bp, Artificial Sequence
  Description of Artificial Sequence: Synthetic oligonucleotide
ccggccctgctcaccggagc

SEQ ID NO: 877 - DNA, 20 bp, Artificial Sequence
  Description of Artificial Sequence: Synthetic oligonucleotide
gcggcggccgacatcgcagt

SEQ ID NO: 878 - DNA, 20 bp, Artificial Sequence
  Description of Artificial Sequence: Synthetic oligonucleotide
ggtgctgatggtgatggcaa

SEQ ID NO: 879 - DNA, 20 bp, Artificial Sequence
  Description of Artificial Sequence: Synthetic oligonucleotide
ctactttgtggtgtcactgg

SEQ ID NO: 880 - DNA, 20 bp, Artificial Sequence
  Description of Artificial Sequence: Synthetic oligonucleotide
tacaccgaggagcccatgat

SEQ ID NO: 881 - DNA, 20 bp, Artificial Sequence
  Description of Artificial Sequence: Synthetic oligonucleotide
tctgtggccatgcccatcat

SEQ ID NO: 882 - DNA, 20 bp, Artificial Sequence
  Description of Artificial Sequence: Synthetic oligonucleotide
attgctgtgctggccatcct

SEQ ID NO: 883 - DNA, 20 bp, Artificial Sequence
  Description of Artificial Sequence: Synthetic oligonucleotide
cgtgaggaccaggacgaagc

SEQ ID NO: 884 - DNA, 20 bp, Artificial Sequence
  Description of Artificial Sequence: Synthetic oligonucleotide
ttgccatcaccatcagcacc

SEQ ID NO: 885 - DNA, 20 bp, Artificial Sequence
  Description of Artificial Sequence: Synthetic oligonucleotide
ggatgcggatggcaatgtag

SEQ ID NO: 886 - DNA, 20 bp, Artificial Sequence
  Description of Artificial Sequence: Synthetic oligonucleotide
ttgccatccgcatcccgctc

SEQ ID NO: 887 - DNA, 20 bp, Artificial Sequence
  Description of Artificial Sequence: Synthetic oligonucleotide
tgaagatggagctctgcgtg

SEQ ID NO: 888 - DNA, 20 bp, Artificial Sequence
  Description of Artificial Sequence: Synthetic oligonucleotide
cattgctgtgctggccatcc

SEQ ID NO: 889 - DNA, 20 bp, Artificial Sequence
  Description of Artificial Sequence: Synthetic oligonucleotide
tgctggtgtgctgggccgtg

SEQ ID NO: 890 - DNA, 21 bp, Artificial Sequence
  Description of Artificial Sequence: Synthetic oligonucleotide
ggctcctcggtgtacatcacg

SEQ ID NO: 891 - DNA, 21 bp, Artificial Sequence
  Description of Artificial Sequence: Synthetic oligonucleotide
gagctctgcgtgaggaccagg

SEQ ID NO: 892 - DNA, 21 bp, Artificial Sequence
  Description of Artificial Sequence: Synthetic oligonucleotide
gatggagctctgcgtgaggac

SEQ ID NO: 893 - DNA, 21 bp, Artificial Sequence
  Description of Artificial Sequence: Synthetic oligonucleotide
ccagcacaccagcacattgcc

SEQ ID NO: 894 - DNA, 21 bp, Artificial Sequence
  Description of Artificial Sequence: Synthetic oligonucleotide
aggaccaggacgaagcaggca

SEQ ID NO: 895 - DNA, 21 bp, Artificial Sequence
  Description of Artificial Sequence: Synthetic oligonucleotide
tgccatccgcatcccgctccg

SEQ ID NO: 896 - DNA, 21 bp, Artificial Sequence
  Description of Artificial Sequence: Synthetic oligonucleotide
gtgtggctcaacagcaacctg

SEQ ID NO: 897 - DNA, 21 bp, Artificial Sequence
  Description of Artificial Sequence: Synthetic oligonucleotide
agctccaccgtgatgtacacc

SEQ ID NO: 898 - DNA, 21 bp, Artificial Sequence
  Description of Artificial Sequence: Synthetic oligonucleotide
gtagcggtcaatggcgatggc

SEQ ID NO: 899 - DNA, 21 bp, Artificial Sequence
  Description of Artificial Sequence: Synthetic oligonucleotide
cggtgctgatggtgatggcaa

SEQ ID NO: 900 - DNA, 21 bp, Artificial Sequence
  Description of Artificial Sequence: Synthetic oligonucleotide
ccctgctcaccggagcgggat

SEQ ID NO: 901 - DNA, 21 bp, Artificial Sequence
  Description of Artificial Sequence: Synthetic oligonucleotide
gtgacgttctgcaggttgctg

SEQ ID NO: 902 - DNA, 21 bp, Artificial Sequence
  Description of Artificial Sequence: Synthetic oligonucleotide
gctccaccgtgatgtacaccg

SEQ ID NO: 903 - DNA, 21 bp, Artificial Sequence
  Description of Artificial Sequence: Synthetic oligonucleotide
actgaagatggagctctgcgt

SEQ ID NO: 904 - DNA, 21 bp, Artificial Sequence
  Description of Artificial Sequence: Synthetic oligonucleotide
ccagctccaccgtgatgtaca

SEQ ID NO: 905 - DNA, 21 bp, Artificial Sequence
  Description of Artificial Sequence: Synthetic oligonucleotide
cctttgccatcaccatcagca

SEQ ID NO: 906 - DNA, 21 bp, Artificial Sequence
  Description of Artificial Sequence: Synthetic oligonucleotide
ccggtgctgatggtgatggca

SEQ ID NO: 907 - DNA, 21 bp, Artificial Sequence
  Description of Artificial Sequence: Synthetic oligonucleotide
cctgggcaatgtgctggtgtg

SEQ ID NO: 908 - DNA, 21 bp, Artificial Sequence
  Description of Artificial Sequence: Synthetic oligonucleotide
aggcagccgtggcaggcagcg

SEQ ID NO: 909 - DNA, 21 bp, Artificial Sequence
  Description of Artificial Sequence: Synthetic oligonucleotide
gcgatggccaggagactgaag

SEQ ID NO: 910 - DNA, 21 bp, Artificial Sequence
  Description of Artificial Sequence: Synthetic oligonucleotide
cgatggccaggagactgaaga

SEQ ID NO: 911 - DNA, 21 bp, Artificial Sequence
  Description of Artificial Sequence: Synthetic oligonucleotide
tcccgctccggtgagcagggc

SEQ ID NO: 912 - DNA, 21 bp, Artificial Sequence
  Description of Artificial Sequence: Synthetic oligonucleotide
tgcttcgtcctggtcctcacg

SEQ ID NO: 913 - DNA, 21 bp, Artificial Sequence
  Description of Artificial Sequence: Synthetic oligonucleotide
accaggacgaagcaggcaatg

SEQ ID NO: 914 - DNA, 21 bp, Artificial Sequence
  Description of Artificial Sequence: Synthetic oligonucleotide
atgtacaccgaggagcccatg

SEQ ID NO: 915 - DNA, 21 bp, Artificial Sequence
  Description of Artificial Sequence: Synthetic oligonucleotide
tcgtctgtggccatgcccatc

SEQ ID NO: 916 - DNA, 21 bp, Artificial Sequence
  Description of Artificial Sequence: Synthetic oligonucleotide
tcaatggcgatggccaggaga

SEQ ID NO: 917 - DNA, 21 bp, Artificial Sequence
  Description of Artificial Sequence: Synthetic oligonucleotide
ggtgctgatggtgatggcaaa

SEQ ID NO: 918 - DNA, 21 bp, Artificial Sequence
  Description of Artificial Sequence: Synthetic oligonucleotide
tagcggtcaatggcgatggcc

SEQ ID NO: 919 - DNA, 21 bp, Artificial Sequence
  Description of Artificial Sequence: Synthetic oligonucleotide
tccgcatcccgctccggtgag

SEQ ID NO: 920 - DNA, 21 bp, Artificial Sequence
  Description of Artificial Sequence: Synthetic oligonucleotide
ctggcggcggccgacatcgca

SEQ ID NO: 921 - DNA, 21 bp, Artificial Sequence
  Description of Artificial Sequence: Synthetic oligonucleotide
gccattgctgtgctggccatc

SEQ ID NO: 922 - DNA, 21 bp, Artificial Sequence
  Description of Artificial Sequence: Synthetic oligonucleotide
atcccgctccggtgagcaggg

SEQ ID NO: 923 - DNA, 21 bp, Artificial Sequence
  Description of Artificial Sequence: Synthetic oligonucleotide
agactgaagatggagctctgc

SEQ ID NO: 924 - DNA, 21 bp, Artificial Sequence
  Description of Artificial Sequence: Synthetic oligonucleotide
ccccggccctgctcaccggag

SEQ ID NO: 925 - DNA, 21 bp, Artificial Sequence
  Description of Artificial Sequence: Synthetic oligonucleotide
atggtgatggcaaaggggatg

SEQ ID NO: 926 - DNA, 21 bp, Artificial Sequence
  Description of Artificial Sequence: Synthetic oligonucleotide
gctcctcggtgtacatcacgg

SEQ ID NO: 927 - DNA, 20 bp, Artificial Sequence
  Description of Artificial Sequence: Synthetic oligonucleotide
tgtcgatggcaatagccaag

SEQ ID NO: 928 - DNA, 20 bp, Artificial Sequence
  Description of Artificial Sequence: Synthetic oligonucleotide
agaagttggtgacgttctgc

SEQ ID NO: 929 - DNA, 20 bp, Artificial Sequence
  Description of Artificial Sequence: Synthetic oligonucleotide
ttcgccatcaccatcagcac

SEQ ID NO: 930 - DNA, 20 bp, Artificial Sequence
  Description of Artificial Sequence: Synthetic oligonucleotide
gaagaagaggcagccatggc

SEQ ID NO: 931 - DNA, 20 bp, Artificial Sequence
  Description of Artificial Sequence: Synthetic oligonucleotide
cacaagcacgttacccagga

SEQ ID NO: 932 - DNA, 20 bp, Artificial Sequence
  Description of Artificial Sequence: Synthetic oligonucleotide
caacttcttcgtggtatctc

SEQ ID NO: 933 - DNA, 20 bp, Artificial Sequence
  Description of Artificial Sequence: Synthetic oligonucleotide
caggatggccagcacagcaa

SEQ ID NO: 934 - DNA, 20 bp, Artificial Sequence
  Description of Artificial Sequence: Synthetic oligonucleotide
aattccactccggtgagcca

SEQ ID NO: 935 - DNA, 20 bp, Artificial Sequence
  Description of Artificial Sequence: Synthetic oligonucleotide
agcgcagaagccagtgctga

SEQ ID NO: 936 - DNA, 20 bp, Artificial Sequence
  Description of Artificial Sequence: Synthetic oligonucleotide
gtgctgatggtgatggcgaa

SEQ ID NO: 937 - DNA, 20 bp, Artificial Sequence
  Description of Artificial Sequence: Synthetic oligonucleotide
ggagctggccattgctgtgc

SEQ ID NO: 938 - DNA, 20 bp, Artificial Sequence
  Description of Artificial Sequence: Synthetic oligonucleotide
aatagccaagaggctgaaga

SEQ ID NO: 939 - DNA, 20 bp, Artificial Sequence
  Description of Artificial Sequence: Synthetic oligonucleotide
ctcctcggtgtacatcatgg

SEQ ID NO: 940 - DNA, 20 bp, Artificial Sequence
  Description of Artificial Sequence: Synthetic oligonucleotide
ggacaaagcaggcgaagaag

SEQ ID NO: 941 - DNA, 20 bp, Artificial Sequence
  Description of Artificial Sequence: Synthetic oligonucleotide
tctggcggcggctgacatcg

SEQ ID NO: 942 - DNA, 20 bp, Artificial Sequence
  Description of Artificial Sequence: Synthetic oligonucleotide
tgggtaacgtgcttgtgtgc

SEQ ID NO: 943 - DNA, 20 bp, Artificial Sequence
  Description of Artificial Sequence: Synthetic oligonucleotide
gatgtacaccgaggagccca

SEQ ID NO: 944 - DNA, 20 bp, Artificial Sequence
  Description of Artificial Sequence: Synthetic oligonucleotide
taacccctggctcaccggag

SEQ ID NO: 945 - DNA, 20 bp, Artificial Sequence
  Description of Artificial Sequence: Synthetic oligonucleotide
tcaccggagtggaattcgga

SEQ ID NO: 946 - DNA, 20 bp, Artificial Sequence
  Description of Artificial Sequence: Synthetic oligonucleotide
gcggcggctgacatcgcggt

SEQ ID NO: 947 - DNA, 20 bp, Artificial Sequence
  Description of Artificial Sequence: Synthetic oligonucleotide
gatggtgatggcgaatggga

SEQ ID NO: 948 - DNA, 20 bp, Artificial Sequence
  Description of Artificial Sequence: Synthetic oligonucleotide
ggcttctgcgctgcctgcca

SEQ ID NO: 949 - DNA, 20 bp, Artificial Sequence
  Description of Artificial Sequence: Synthetic oligonucleotide
attccactccggtgagccag

SEQ ID NO: 950 - DNA, 20 bp, Artificial Sequence
  Description of Artificial Sequence: Synthetic oligonucleotide
ggtgtacatcatggtggagc

SEQ ID NO: 951 - DNA, 20 bp, Artificial Sequence
  Description of Artificial Sequence: Synthetic oligonucleotide
attgctgtgctggccatcct

SEQ ID NO: 952 - DNA, 20 bp, Artificial Sequence
  Description of Artificial Sequence: Synthetic oligonucleotide
ctccaccatgatgtacaccg

SEQ ID NO: 953 - DNA, 20 bp, Artificial Sequence
  Description of Artificial Sequence: Synthetic oligonucleotide
ggcggcggctgacatcgcgg

SEQ ID NO: 954 - DNA, 20 bp, Artificial Sequence
  Description of Artificial Sequence: Synthetic oligonucleotide
tacaccgaggagcccatggc

SEQ ID NO: 955 - DNA, 20 bp, Artificial Sequence
  Description of Artificial Sequence: Synthetic oligonucleotide
gggtaacgtgcttgtgtgct

SEQ ID NO: 956 - DNA, 20 bp, Artificial Sequence
  Description of Artificial Sequence: Synthetic oligonucleotide
caggttgctgttgatccaca

SEQ ID NO: 957 - DNA, 20 bp, Artificial Sequence
  Description of Artificial Sequence: Synthetic oligonucleotide
tgaagatggaactctgcgtg

SEQ ID NO: 958 - DNA, 20 bp, Artificial Sequence
  Description of Artificial Sequence: Synthetic oligonucleotide
gatggcgatgtatctgtcga

SEQ ID NO: 959 - DNA, 20 bp, Artificial Sequence
  Description of Artificial Sequence: Synthetic oligonucleotide
cttcttcgcctgctttgtcc

SEQ ID NO: 960 - DNA, 20 bp, Artificial Sequence
  Description of Artificial Sequence: Synthetic oligonucleotide
aggcgaagaagaggcagcca

SEQ ID NO: 961 - DNA, 20 bp, Artificial Sequence
  Description of Artificial Sequence: Synthetic oligonucleotide
tgcttgtgtgctgggccgtg

SEQ ID NO: 962 - DNA, 20 bp, Artificial Sequence
  Description of Artificial Sequence: Synthetic oligonucleotide
gaagccagtgctgatggtga

SEQ ID NO: 963 - DNA, 20 bp, Artificial Sequence
  Description of Artificial Sequence: Synthetic oligonucleotide
cgtgaggaccaggacaaagc

SEQ ID NO: 964 - DNA, 20 bp, Artificial Sequence
  Description of Artificial Sequence: Synthetic oligonucleotide
tggaactctgcgtgaggacc

SEQ ID NO: 965 - DNA, 20 bp, Artificial Sequence
  Description of Artificial Sequence: Synthetic oligonucleotide
cattgctgtgctggccatcc

SEQ ID NO: 966 - DNA, 20 bp, Artificial Sequence
  Description of Artificial Sequence: Synthetic oligonucleotide
ttctcccgccatgggctcct

SEQ ID NO: 967 - DNA, 20 bp, Artificial Sequence
  Description of Artificial Sequence: Synthetic oligonucleotide
tggctcaccggagtggaatt

SEQ ID NO: 968 - DNA, 20 bp, Artificial Sequence
  Description of Artificial Sequence: Synthetic oligonucleotide
tgctgatggtgatggcgaat

SEQ ID NO: 969 - DNA, 20 bp, Artificial Sequence
  Description of Artificial Sequence: Synthetic oligonucleotide
cttcgtggtatctctggcgg

SEQ ID NO: 970 - DNA, 20 bp, Artificial Sequence
  Description of Artificial Sequence: Synthetic oligonucleotide
agcacacaagcacgttaccc

SEQ ID NO: 971 - DNA, 20 bp, Artificial Sequence
  Description of Artificial Sequence: Synthetic oligonucleotide
gggctcctcggtgtacatca

SEQ ID NO: 972 - DNA, 20 bp, Artificial Sequence
  Description of Artificial Sequence: Synthetic oligonucleotide
gtacaccgaggagcccatgg

SEQ ID NO: 973 - DNA, 20 bp, Artificial Sequence
  Description of Artificial Sequence: Synthetic oligonucleotide
gaacgtcaccaacttcttcg

SEQ ID NO: 974 - DNA, 20 bp, Artificial Sequence
  Description of Artificial Sequence: Synthetic oligonucleotide
tcgccatccgaattccactc

SEQ ID NO: 975 - DNA, 20 bp, Artificial Sequence
  Description of Artificial Sequence: Synthetic oligonucleotide
gagttccatcttcagcctct

SEQ ID NO: 976 - DNA, 20 bp, Artificial Sequence
  Description of Artificial Sequence: Synthetic oligonucleotide
gaattccactccggtgagcc

SEQ ID NO: 977 - DNA, 20 bp, Artificial Sequence
  Description of Artificial Sequence: Synthetic oligonucleotide
cagagataccacgaagaagt

SEQ ID NO: 978 - DNA, 20 bp, Artificial Sequence
  Description of Artificial Sequence: Synthetic oligonucleotide
cttcttcgtggtatctctgg

SEQ ID NO: 979 - DNA, 21 bp, Artificial Sequence
  Description of Artificial Sequence: Synthetic oligonucleotide
cagtgctgatggtgatggcga

SEQ ID NO: 980 - DNA, 21 bp, Artificial Sequence
  Description of Artificial Sequence: Synthetic oligonucleotide
cgaattccactccggtgagcc

SEQ ID NO: 981 - DNA, 21 bp, Artificial Sequence
  Description of Artificial Sequence: Synthetic oligonucleotide
ccgaattccactccggtgagc

SEQ ID NO: 982 - DNA, 21 bp, Artificial Sequence
  Description of Artificial Sequence: Synthetic oligonucleotide
gctgaagatggaactctgcgt

SEQ ID NO: 983 - DNA, 21 bp, Artificial Sequence
  Description of Artificial Sequence: Synthetic oligonucleotide
cgtgcttgtgtgctgggccgt

SEQ ID NO: 984 - DNA, 21 bp, Artificial Sequence
  Description of Artificial Sequence: Synthetic oligonucleotide
gtgaggaccaggacaaagcag

SEQ ID NO: 985 - DNA, 21 bp, Artificial Sequence
  Description of Artificial Sequence: Synthetic oligonucleotide
tcgatggcaatagccaagagg

SEQ ID NO: 986 - DNA, 21 bp, Artificial Sequence
  Description of Artificial Sequence: Synthetic oligonucleotide
catcgacagatacatcgccat

SEQ ID NO: 987 - DNA, 21 bp, Artificial Sequence
  Description of Artificial Sequence: Synthetic oligonucleotide
gtacaccgaggagcccatggc

SEQ ID NO: 988 - DNA, 21 bp, Artificial Sequence
  Description of Artificial Sequence: Synthetic oligonucleotide
gctccaccatgatgtacaccg

SEQ ID NO: 989 - DNA, 21 bp, Artificial Sequence
  Description of Artificial Sequence: Synthetic oligonucleotide
aagccagtgctgatggtgatg

SEQ ID NO: 990 - DNA, 21 bp, Artificial Sequence
  Description of Artificial Sequence: Synthetic oligonucleotide
caccgcgatgtcagccgccgc

SEQ ID NO: 991 - DNA, 21 bp, Artificial Sequence
  Description of Artificial Sequence: Synthetic oligonucleotide
aggctgaagatggaactctgc

SEQ ID NO: 992 - DNA, 21 bp, Artificial Sequence
  Description of Artificial Sequence: Synthetic oligonucleotide
gccgccgccagagataccacg

SEQ ID NO: 993 - DNA, 21 bp, Artificial Sequence
  Description of Artificial Sequence: Synthetic oligonucleotide
agctccaccatgatgtacacc

SEQ ID NO: 994 - DNA, 21 bp, Artificial Sequence
  Description of Artificial Sequence: Synthetic oligonucleotide
aggcagccatggcaggcagcg

SEQ ID NO: 995 - DNA, 21 bp, Artificial Sequence
  Description of Artificial Sequence: Synthetic oligonucleotide
cctggctcaccggagtggaat

SEQ ID NO: 996 - DNA, 21 bp, Artificial Sequence
  Description of Artificial Sequence: Synthetic oligonucleotide
ccagctccaccatgatgtaca

SEQ ID NO: 997 - DNA, 21 bp, Artificial Sequence
  Description of Artificial Sequence: Synthetic oligonucleotide
accaggacaaagcaggcgaag

SEQ ID NO: 998 - DNA, 21 bp, Artificial Sequence
  Description of Artificial Sequence: Synthetic oligonucleotide
cctgggtaacgtgcttgtgtg

SEQ ID NO: 999 - DNA, 21 bp, Artificial Sequence
  Description of Artificial Sequence: Synthetic oligonucleotide
aggaccaggacaaagcaggcg

SEQ ID NO: 1000 - DNA, 21 bp, Artificial Sequence
  Description of Artificial Sequence: Synthetic oligonucleotide
tcagccgccgccagagatacc

SEQ ID NO: 1001 - DNA, 21 bp, Artificial Sequence
  Description of Artificial Sequence: Synthetic oligonucleotide
ggctcctcggtgtacatcatg

SEQ ID NO: 1002 - DNA, 21 bp, Artificial Sequence
  Description of Artificial Sequence: Synthetic oligonucleotide
ctggcggcggctgacatcgcg

SEQ ID NO: 1003 - DNA, 21 bp, Artificial Sequence
  Description of Artificial Sequence: Synthetic oligonucleotide
gatggaactctgcgtgaggac

SEQ ID NO: 1004 - DNA, 21 bp, Artificial Sequence
  Description of Artificial Sequence: Synthetic oligonucleotide
gctcctcggtgtacatcatgg

SEQ ID NO: 1005 - DNA, 21 bp, Artificial Sequence
  Description of Artificial Sequence: Synthetic oligonucleotide
tgtacaccgaggagcccatgg

SEQ ID NO: 1006 - DNA, 21 bp, Artificial Sequence
  Description of Artificial Sequence: Synthetic oligonucleotide
gccattgctgtgctggccatc

SEQ ID NO: 1007 - DNA, 21 bp, Artificial Sequence
  Description of Artificial Sequence: Synthetic oligonucleotide
caatagccaagaggctgaaga

SEQ ID NO: 1008 - DNA, 21 bp, Artificial Sequence
  Description of Artificial Sequence: Synthetic oligonucleotide
atggtgatggcgaatgggatg

SEQ ID NO: 1009 - DNA, 21 bp, Artificial Sequence
  Description of Artificial Sequence: Synthetic oligonucleotide
atgtacaccgaggagcccatg

SEQ ID NO: 1010 - DNA, 21 bp, Artificial Sequence
  Description of Artificial Sequence: Synthetic oligonucleotide
gtgtggatcaacagcaacctg

SEQ ID NO: 1011 - DNA, 21 bp, Artificial Sequence
  Description of Artificial Sequence: Synthetic oligonucleotide
tgctttgtcctggtcctcacg

SEQ ID NO: 1012 - DNA, 21 bp, Artificial Sequence
  Description of Artificial Sequence: Synthetic oligonucleotide
gtaacccctggctcaccggag

SEQ ID NO: 1013 - DNA, 21 bp, Artificial Sequence
  Description of Artificial Sequence: Synthetic oligonucleotide
ccagcacacaagcacgttacc

SEQ ID NO: 1014 - DNA, 21 bp, Artificial Sequence
  Description of Artificial Sequence: Synthetic oligonucleotide
tatctgtcgatggcaatagcc

SEQ ID NO: 1015 - DNA, 21 bp, Artificial Sequence
  Description of Artificial Sequence: Synthetic oligonucleotide
gcaatagccaagaggctgaag

SEQ ID NO: 1016 - DNA, 21 bp, Artificial Sequence
  Description of Artificial Sequence: Synthetic oligonucleotide
agtgctgatggtgatggcgaa

SEQ ID NO: 1017 - DNA, 21 bp, Artificial Sequence
  Description of Artificial Sequence: Synthetic oligonucleotide
acaccgaggagcccatggcgg

SEQ ID NO: 1018 - DNA, 21 bp, Artificial Sequence
  Description of Artificial Sequence: Synthetic oligonucleotide
cgccatccgaattccactccg

SEQ ID NO: 1019 - DNA, 20 bp, Artificial Sequence
  Description of Artificial Sequence: Synthetic oligonucleotide
tggtgtcactggcggcggcc

SEQ ID NO: 1020 - DNA, 20 bp, Artificial Sequence
  Description of Artificial Sequence: Synthetic oligonucleotide
ccatcaccatcagcaccggg

SEQ ID NO: 1021 - DNA, 20 bp, Artificial Sequence
  Description of Artificial Sequence: Synthetic oligonucleotide
ccatcggcctgactcccatg

SEQ ID NO: 1022 - DNA, 20 bp, Artificial Sequence
  Description of Artificial Sequence: Synthetic oligonucleotide
gctgaccgcagttgttccaa

SEQ ID NO: 1023 - DNA, 20 bp, Artificial Sequence
  Description of Artificial Sequence: Synthetic oligonucleotide
aggatgtggtccccatgaac

SEQ ID NO: 1024 - DNA, 20 bp, Artificial Sequence
  Description of Artificial Sequence: Synthetic oligonucleotide
cctgtgtgctggtgcccctg

SEQ ID NO: 1025 - DNA, 20 bp, Artificial Sequence
  Description of Artificial Sequence: Synthetic oligonucleotide
cggatcttcctggcggcgcg

SEQ ID NO: 1026 - DNA, 20 bp, Artificial Sequence
  Description of Artificial Sequence: Synthetic oligonucleotide
ccctctgctggctgccccta

SEQ ID NO: 1027 - DNA, 20 bp, Artificial Sequence
  Description of Artificial Sequence: Synthetic oligonucleotide
ttctgccccgactgcagcca

SEQ ID NO: 1028 - DNA, 20 bp, Artificial Sequence
  Description of Artificial Sequence: Synthetic oligonucleotide
aaggcagctggcaccagtgc

SEQ ID NO: 1029 - DNA, 21 bp, Artificial Sequence
  Description of Artificial Sequence: Synthetic oligonucleotide
taagggcatcattgccatctg

SEQ ID NO: 1030 - DNA, 21 bp, Artificial Sequence
  Description of Artificial Sequence: Synthetic oligonucleotide
cggcctgactcccatgctagg

SEQ ID NO: 1031 - DNA, 21 bp, Artificial Sequence
  Description of Artificial Sequence: Synthetic oligonucleotide
gcagttgttccaacctagcat

SEQ ID NO: 1032 - DNA, 21 bp, Artificial Sequence
  Description of Artificial Sequence: Synthetic oligonucleotide
ccgcagttgttccaacctagc

SEQ ID NO: 1033 - DNA, 21 bp, Artificial Sequence
  Description of Artificial Sequence: Synthetic oligonucleotide
caagaaccactcccagggctg

SEQ ID NO: 1034 - DNA, 21 bp, Artificial Sequence
  Description of Artificial Sequence: Synthetic oligonucleotide
cttggccctccccgcagccct

SEQ ID NO: 1035 - DNA, 21 bp, Artificial Sequence
  Description of Artificial Sequence: Synthetic oligonucleotide
cacttggccctccccgcagcc

SEQ ID NO: 1036 - DNA, 21 bp, Artificial Sequence
  Description of Artificial Sequence: Synthetic oligonucleotide
ggccaagtggcctgtctcttt

SEQ ID NO: 1037 - DNA, 21 bp, Artificial Sequence
  Description of Artificial Sequence: Synthetic oligonucleotide
ttcatggggaccacatcctca

SEQ ID NO: 1038 - DNA, 21 bp, Artificial Sequence
  Description of Artificial Sequence: Synthetic oligonucleotide
tgaagtacaccatgtagttca

SEQ ID NO: 1039 - DNA, 21 bp, Artificial Sequence
  Description of Artificial Sequence: Synthetic oligonucleotide
ctggtgcccctgctgctcatg

SEQ ID NO: 1040 - DNA, 21 bp, Artificial Sequence
  Description of Artificial Sequence: Synthetic oligonucleotide
gctcatgctgggtgtctattt

SEQ ID NO: 1041 - DNA, 21 bp, Artificial Sequence
  Description of Artificial Sequence: Synthetic oligonucleotide
cttcagctgtcgtcgcgccgc

SEQ ID NO: 1042 - DNA, 21 bp, Artificial Sequence
  Description of Artificial Sequence: Synthetic oligonucleotide
cgcgacgacagctgaagcaga

SEQ ID NO: 1043 - DNA, 21 bp, Artificial Sequence
  Description of Artificial Sequence: Synthetic oligonucleotide
gatggagagccagcctctgcc

SEQ ID NO: 1044 - DNA, 21 bp, Artificial Sequence
  Description of Artificial Sequence: Synthetic oligonucleotide
gcgtggctgcagtcggggcag

SEQ ID NO: 1045 - DNA, 21 bp, Artificial Sequence
  Description of Artificial Sequence: Synthetic oligonucleotide
acgatggccaggtacatgagc

SEQ ID NO: 1046 - DNA, 21 bp, Artificial Sequence
  Description of Artificial Sequence: Synthetic oligonucleotide
ctctcccacaccaattcggtt

SEQ ID NO: 1047 - DNA, 21 bp, Artificial Sequence
  Description of Artificial Sequence: Synthetic oligonucleotide
gattcacaaccgaattggtgt

SEQ ID NO: 1048 - DNA, 21 bp, Artificial Sequence
  Description of Artificial Sequence: Synthetic oligonucleotide
gggattcacaaccgaattggt

SEQ ID NO: 1049 - DNA, 21 bp, Artificial Sequence
  Description of Artificial Sequence: Synthetic oligonucleotide
cgtagatgaagggattcacaa

SEQ ID NO: 1050 - DNA, 21 bp, Artificial Sequence
  Description of Artificial Sequence: Synthetic oligonucleotide
ggatacggtaggcgtagatga

SEQ ID NO: 1051 - DNA, 21 bp, Artificial Sequence
  Description of Artificial Sequence: Synthetic oligonucleotide
tcatctacgcctaccgtatcc

SEQ ID NO: 1052 - DNA, 21 bp, Artificial Sequence
  Description of Artificial Sequence: Synthetic oligonucleotide
cggatacggtaggcgtagatg

SEQ ID NO: 1053 - DNA, 21 bp, Artificial Sequence
  Description of Artificial Sequence: Synthetic oligonucleotide
gcggaaggtctggcggaactc

SEQ ID NO: 1054 - DNA, 21 bp, Artificial Sequence
  Description of Artificial Sequence: Synthetic oligonucleotide
aatgatcttgcggaaggtctg

SEQ ID NO: 1055 - DNA, 21 bp, Artificial Sequence
  Description of Artificial Sequence: Synthetic oligonucleotide
gacgtggctgcgaatgatctt

SEQ ID NO: 1056 - DNA, 21 bp, Artificial Sequence
  Description of Artificial Sequence: Synthetic oligonucleotide
ttgctgcctcaggacgtggct

SEQ ID NO: 1057 - DNA, 21 bp, Artificial Sequence
  Description of Artificial Sequence: Synthetic oligonucleotide
caaggcagctggcaccagtgc

SEQ ID NO: 1058 - DNA, 21 bp, Artificial Sequence
  Description of Artificial Sequence: Synthetic oligonucleotide
cgggcactggtgccagctgcc

SEQ ID NO: 1059 - DNA, 21 bp, Artificial Sequence
  Description of Artificial Sequence: Synthetic oligonucleotide
cttggcagctcatggcagtga

SEQ ID NO: 1060 - DNA, 21 bp, Artificial Sequence
  Description of Artificial Sequence: Synthetic oligonucleotide
ccgtctcaacggccacccgcc

SEQ ID NO: 1061 - DNA, 21 bp, Artificial Sequence
  Description of Artificial Sequence: Synthetic oligonucleotide
cacactcctggcgggtggccg

SEQ ID NO: 1062 - DNA, 21 bp, Artificial Sequence
  Description of Artificial Sequence: Synthetic oligonucleotide
tgccgttggcccacactcctg

SEQ ID NO: 1063 - DNA, 21 bp, Artificial Sequence
  Description of Artificial Sequence: Synthetic oligonucleotide
ccattgggcctccgctcaggg

SEQ ID NO: 1064 - DNA, 21 bp, Artificial Sequence
  Description of Artificial Sequence: Synthetic oligonucleotide
catagccattgggcctccgct

SEQ ID NO: 1065 - DNA, 21 bp, Artificial Sequence
  Description of Artificial Sequence: Synthetic oligonucleotide
aatggctatgccctggggctg

SEQ ID NO: 1066 - DNA, 21 bp, Artificial Sequence
  Description of Artificial Sequence: Synthetic oligonucleotide
atgccctggggctggtgagtg

SEQ ID NO: 1067 - DNA, 21 bp, Artificial Sequence
  Description of Artificial Sequence: Synthetic oligonucleotide
gccctggggctggtgagtgga

SEQ ID NO: 1068 - DNA, 21 bp, Artificial Sequence
  Description of Artificial Sequence: Synthetic oligonucleotide
tggtgagtggagggagtgccc

SEQ ID NO: 1069 - DNA, 21 bp, Artificial Sequence
  Description of Artificial Sequence: Synthetic oligonucleotide
gagggagtgcccaagagtccc

SEQ ID NO: 1070 - DNA, 21 bp, Artificial Sequence
  Description of Artificial Sequence: Synthetic oligonucleotide
agggagtgcccaagagtccca

SEQ ID NO: 1071 - DNA, 21 bp, Artificial Sequence
  Description of Artificial Sequence: Synthetic oligonucleotide
gtctgggaggcccgtgttccc

SEQ ID NO: 1072 - DNA, 21 bp, Artificial Sequence
  Description of Artificial Sequence: Synthetic oligonucleotide
catggctaaggagctccacgt

SEQ ID NO: 1073 - DNA, 21 bp, Artificial Sequence
  Description of Artificial Sequence: Synthetic oligonucleotide
gagctccttagccatgagctc

SEQ ID NO: 1074 - DNA, 21 bp, Artificial Sequence
  Description of Artificial Sequence: Synthetic oligonucleotide
gctccttagccatgagctcaa

SEQ ID NO: 1075 - DNA, 21 bp, Artificial Sequence
  Description of Artificial Sequence: Synthetic oligonucleotide
ggcctagatgaccccctggcc

SEQ ID NO: 1076 - DNA, 21 bp, Artificial Sequence
  Description of Artificial Sequence: Synthetic oligonucleotide
ccccctggcccaggatggagc

SEQ ID NO: 1077 - DNA, 21 bp, Artificial Sequence
  Description of Artificial Sequence: Synthetic oligonucleotide
ctcctgctccatcctgggcca

SEQ ID NO: 1078 - DNA, 20 bp, Artificial Sequence
  Description of Artificial Sequence: Synthetic oligonucleotide
ccgtgatgtacaccgaggag

SEQ ID NO: 1079 - DNA, 20 bp, Artificial Sequence
  Description of Artificial Sequence: Synthetic oligonucleotide
ctttgccatcaccatcagca

SEQ ID NO: 1080 - DNA, 20 bp, Artificial Sequence
  Description of Artificial Sequence: Synthetic oligonucleotide
tttgccatcaccatcagcac

SEQ ID NO: 1081 - DNA, 20 bp, Artificial Sequence
  Description of Artificial Sequence: Synthetic oligonucleotide
ttgcctgcttcgtcctggtc

SEQ ID NO: 1082 - DNA, 20 bp, Artificial Sequence
  Description of Artificial Sequence: Synthetic oligonucleotide
tcctggtcctcacgcagagc

SEQ ID NO: 1083 - DNA, 20 bp, Artificial Sequence
  Description of Artificial Sequence: Synthetic oligonucleotide
tcttcagtctcctggccatc

SEQ ID NO: 1084 - DNA, 20 bp, Artificial Sequence
  Description of Artificial Sequence: Synthetic oligonucleotide
gtctcctggccatcgccatt

SEQ ID NO: 1085 - DNA, 20 bp, Artificial Sequence
  Description of Artificial Sequence: Synthetic oligonucleotide
acctagcatgggagtcaggc

SEQ ID NO: 1086 - DNA, 20 bp, Artificial Sequence
  Description of Artificial Sequence: Synthetic oligonucleotide
aacctagcatgggagtcagg

SEQ ID NO: 1087 - DNA, 20 bp, Artificial Sequence
  Description of Artificial Sequence: Synthetic oligonucleotide
atgctaggttggaacaactg

SEQ ID NO: 1088 - DNA, 20 bp, Artificial Sequence
  Description of Artificial Sequence: Synthetic oligonucleotide
gcagccctgggagtggttct

SEQ ID NO: 1089 - DNA, 20 bp, Artificial Sequence
  Description of Artificial Sequence: Synthetic oligonucleotide
cgcagccctgggagtggttc

SEQ ID NO: 1090 - DNA, 20 bp, Artificial Sequence
  Description of Artificial Sequence: Synthetic oligonucleotide
agggctgcggggagggccaa

SEQ ID NO: 1091 - DNA, 20 bp, Artificial Sequence
  Description of Artificial Sequence: Synthetic oligonucleotide
tggggaccacatcctcaaag

SEQ ID NO: 1092 - DNA, 20 bp, Artificial Sequence
  Description of Artificial Sequence: Synthetic oligonucleotide
catgaactacatggtgtact

SEQ ID NO: 1093 - DNA, 20 bp, Artificial Sequence
  Description of Artificial Sequence: Synthetic oligonucleotide
atgaactacatggtgtactt

SEQ ID NO: 1094 - DNA, 20 bp, Artificial Sequence
  Description of Artificial Sequence: Synthetic oligonucleotide
acttctttgcctgtgtgctg

SEQ ID NO: 1095 - DNA, 20 bp, Artificial Sequence
  Description of Artificial Sequence: Synthetic oligonucleotide
tgctgctcatgctgggtgtc

SEQ ID NO: 1096 - DNA, 20 bp, Artificial Sequence
  Description of Artificial Sequence: Synthetic oligonucleotide
caaatagacacccagcatga

SEQ ID NO: 1097 - DNA, 20 bp, Artificial Sequence
  Description of Artificial Sequence: Synthetic oligonucleotide
gctgtcgtcgcgccgccagg

SEQ ID NO: 1098 - DNA, 20 bp, Artificial Sequence
  Description of Artificial Sequence: Synthetic oligonucleotide
tggcggcgcgacgacagctg

SEQ ID NO: 1099 - DNA, 20 bp, Artificial Sequence
  Description of Artificial Sequence: Synthetic oligonucleotide
tctgcttcagctgtcgtcgc

SEQ ID NO: 1100 - DNA, 20 bp, Artificial Sequence
  Description of Artificial Sequence: Synthetic oligonucleotide
ggcagaggctggctctccat

SEQ ID NO: 1101 - DNA, 20 bp, Artificial Sequence
  Description of Artificial Sequence: Synthetic oligonucleotide
cggcagaggctggctctcca

SEQ ID NO: 1102 - DNA, 20 bp, Artificial Sequence
  Description of Artificial Sequence: Synthetic oligonucleotide
ccggcagaggctggctctcc

SEQ ID NO: 1103 - DNA, 20 bp, Artificial Sequence
  Description of Artificial Sequence: Synthetic oligonucleotide
cactgcagaaggaggtccat

SEQ ID NO: 1104 - DNA, 20 bp, Artificial Sequence
  Description of Artificial Sequence: Synthetic oligonucleotide
tgctgccaagtcactggcca

SEQ ID NO: 1105 - DNA, 20 bp, Artificial Sequence
  Description of Artificial Sequence: Synthetic oligonucleotide
acaatgatggccagtgactt

SEQ ID NO: 1106 - DNA, 20 bp, Artificial Sequence
  Description of Artificial Sequence: Synthetic oligonucleotide
tacacatcatcaactgcttc

SEQ ID NO: 1107 - DNA, 20 bp, Artificial Sequence
  Description of Artificial Sequence: Synthetic oligonucleotide
ctttcttctgccccgactgc

SEQ ID NO: 1108 - DNA, 20 bp, Artificial Sequence
  Description of Artificial Sequence: Synthetic oligonucleotide
gactgcagccacgcccctct

SEQ ID NO: 1109 - DNA, 20 bp, Artificial Sequence
  Description of Artificial Sequence: Synthetic oligonucleotide
tctctggctcatgtacctgg

SEQ ID NO: 1110 - DNA, 20 bp, Artificial Sequence
  Description of Artificial Sequence: Synthetic oligonucleotide
caaccgaattggtgtgggag

SEQ ID NO: 1111 - DNA, 20 bp, Artificial Sequence
  Description of Artificial Sequence: Synthetic oligonucleotide
acaccaattcggttgtgaat

SEQ ID NO: 1112 - DNA, 20 bp, Artificial Sequence
  Description of Artificial Sequence: Synthetic oligonucleotide
gttgtgaatcccttcatcta

SEQ ID NO: 1113 - DNA, 20 bp, Artificial Sequence
  Description of Artificial Sequence: Synthetic oligonucleotide
ttcatctacgcctaccgtat

SEQ ID NO: 1114 - DNA, 20 bp, Artificial Sequence
  Description of Artificial Sequence: Synthetic oligonucleotide
tctacgcctaccgtatccgc

SEQ ID NO: 1115 - DNA, 20 bp, Artificial Sequence
  Description of Artificial Sequence: Synthetic oligonucleotide
cgagttccgccagaccttcc

SEQ ID NO: 1116 - DNA, 20 bp, Artificial Sequence
  Description of Artificial Sequence: Synthetic oligonucleotide
gccagaccttccgcaagatc

SEQ ID NO: 1117 - DNA, 20 bp, Artificial Sequence
  Description of Artificial Sequence: Synthetic oligonucleotide
ccagaccttccgcaagatca

SEQ ID NO: 1118 - DNA, 20 bp, Artificial Sequence
  Description of Artificial Sequence: Synthetic oligonucleotide
gcaagatcattcgcagccac

SEQ ID NO: 1119 - DNA, 20 bp, Artificial Sequence
  Description of Artificial Sequence: Synthetic oligonucleotide
caagatcattcgcagccacg

SEQ ID NO: 1120 - DNA, 20 bp, Artificial Sequence
  Description of Artificial Sequence: Synthetic oligonucleotide
cagccacgtcctgaggcagc

SEQ ID NO: 1121 - DNA, 20 bp, Artificial Sequence
  Description of Artificial Sequence: Synthetic oligonucleotide
aggcagctggcaccagtgcc

SEQ ID NO: 1122 - DNA, 20 bp, Artificial Sequence
  Description of Artificial Sequence: Synthetic oligonucleotide
tcactgccatgagctgccaa

SEQ ID NO: 1123 - DNA, 20 bp, Artificial Sequence
  Description of Artificial Sequence: Synthetic oligonucleotide
tctcaacggccacccgccag

SEQ ID NO: 1124 - DNA, 20 bp, Artificial Sequence
  Description of Artificial Sequence: Synthetic oligonucleotide
ctcagggtggggagcactgc

SEQ ID NO: 1125 - DNA, 20 bp, Artificial Sequence
  Description of Artificial Sequence: Synthetic oligonucleotide
caccctgagcggaggcccaa

SEQ ID NO: 1126 - DNA, 20 bp, Artificial Sequence
  Description of Artificial Sequence: Synthetic oligonucleotide
accctgagcggaggcccaat

SEQ ID NO: 1127 - DNA, 20 bp, Artificial Sequence
  Description of Artificial Sequence: Synthetic oligonucleotide
agggcatagccattgggcct

SEQ ID NO: 1128 - DNA, 20 bp, Artificial Sequence
  Description of Artificial Sequence: Synthetic oligonucleotide
ctcaccagccccagggcata

SEQ ID NO: 1129 - DNA, 20 bp, Artificial Sequence
  Description of Artificial Sequence: Synthetic oligonucleotide
tccactcaccagccccaggg

SEQ ID NO: 1130 - DNA, 20 bp, Artificial Sequence
  Description of Artificial Sequence: Synthetic oligonucleotide
tgggactcttgggcactccc

SEQ ID NO: 1131 - DNA, 20 bp, Artificial Sequence
  Description of Artificial Sequence: Synthetic oligonucleotide
ctgggactcttgggcactcc

SEQ ID NO: 1132 - DNA, 20 bp, Artificial Sequence
  Description of Artificial Sequence: Synthetic oligonucleotide
cctgggactcttgggcactc

SEQ ID NO: 1133 - DNA, 20 bp, Artificial Sequence
  Description of Artificial Sequence: Synthetic oligonucleotide
aggggaacacgggcctccca

SEQ ID NO: 1134 - DNA, 20 bp, Artificial Sequence
  Description of Artificial Sequence: Synthetic oligonucleotide
cgtctgggaggcccgtgttc

SEQ ID NO: 1135 - DNA, 20 bp, Artificial Sequence
  Description of Artificial Sequence: Synthetic oligonucleotide
agacgtggagctccttagcc

SEQ ID NO: 1136 - DNA, 20 bp, Artificial Sequence
  Description of Artificial Sequence: Synthetic oligonucleotide
ttgagctcatggctaaggag

SEQ ID NO: 1137 - DNA, 20 bp, Artificial Sequence
  Description of Artificial Sequence: Synthetic oligonucleotide
ctggcctagatgaccccctg

SEQ ID NO: 1138 - DNA, 20 bp, Artificial Sequence
  Description of Artificial Sequence: Synthetic oligonucleotide
tggcctagatgaccccctgg

SEQ ID NO: 1139 - DNA, 20 bp, Artificial Sequence
  Description of Artificial Sequence: Synthetic oligonucleotide
tcctgggccagggggtcatc

SEQ ID NO: 1140 - DNA, 20 bp, Artificial Sequence
  Description of Artificial Sequence: Synthetic oligonucleotide
ctggcccaggatggagcagg

SEQ ID NO: 1141 - DNA, 20 bp, Artificial Sequence
  Description of Artificial Sequence: Synthetic oligonucleotide
tggcccaggatggagcagga

SEQ ID NO: 1142 - DNA, 20 bp, Artificial Sequence
  Description of Artificial Sequence: Synthetic oligonucleotide
cgcgagttccgccagacctt

SEQ ID NO: 1143 - DNA, 20 bp, Artificial Sequence
  Description of Artificial Sequence: Synthetic oligonucleotide
ccctggggctggtgagtgga

SEQ ID NO: 1144 - protein, 1334 aa, Artificial Sequence
  Description of Artificial Sequence: Synthetic polypeptide
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1145 - protein, 1363 aa, Artificial Sequence
  Description of Artificial Sequence: Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1146 - protein, 1363 aa, Artificial Sequence
  Description of Artificial Sequence: Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1147 - protein, 1363 aa, Artificial Sequence
  Description of Artificial Sequence: Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1148 - protein, 1331 aa, Artificial Sequence
  Description of Artificial Sequence: Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1149 - protein, 1331 aa, Artificial Sequence
  Description of Artificial Sequence: Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1150 - protein, 1363 aa, Artificial Sequence; identical to SEQ ID NO: 1145
  Description of Artificial Sequence: Synthetic polypeptide

SEQ ID NO: 1151 - protein, 1361 aa, Artificial Sequence
  Description of Artificial Sequence: Synthetic polypeptide
MGRDPGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 1152 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 1153 - RNA, 20 nt, Artificial Sequence
  Description of Artificial Sequence: Synthetic oligonucleotide
uaauuucuacucuuguagau

SEQ ID NO: 1154 - DNA, 25 bp, Artificial Sequence
  Description of Artificial Sequence: Synthetic oligonucleotide
atgtgtttttgtcaaaagacctttt

SEQ ID NO: 1155 - RNA, 20 nt, Artificial Sequence
  Description of Artificial Sequence: Synthetic oligonucleotide
gguguacagcaguggcuggu

SEQ ID NO: 1156 - DNA, 65 bp, Artificial Sequence
  Description of Artificial Sequence: Synthetic oligonucleotide
  Description of Combined DNA/RNA Molecule: Synthetic oligonucleotide
atgtgtttttgtcaaaagaccttttuaauuucuacucuuguagaugguguacagcaguggcuggu

SEQ ID NO: 1157 - RNA, 20 nt, Artificial Sequence
  Description of Artificial Sequence: Synthetic oligonucleotide
ugaugugagauuuuccaccu

SEQ ID NO: 1158 - DNA, 65 bp, Artificial Sequence
  Description of Artificial Sequence: Synthetic oligonucleotide
  Description of Combined DNA/RNA Molecule: Synthetic oligonucleotide
atgtgtttttgtcaaaagaccttttuaauuucuacucuuguagauugaugugagauuuuccaccu

SEQ ID NO: 1159 - RNA, 20 nt, Artificial Sequence
  Description of Artificial Sequence: Synthetic oligonucleotide
ccaucggccugacucccaug

SEQ ID NO: 1160 - DNA, 65 bp, Artificial Sequence
  Description of Artificial Sequence: Synthetic oligonucleotide
  Description of Combined DNA/RNA Molecule: Synthetic oligonucleotide
atgtgtttttgtcaaaagaccttttuaauuucuacucuuguagauccaucggccugacucccaug

SEQ ID NO: 1161 - RNA, 21 nt, Artificial Sequence
  Description of Artificial Sequence: Synthetic oligonucleotide
ugaugugagauuuuccaccug

SEQ ID NO: 1162 - RNA, 21 nt, Artificial Sequence
  Description of Artificial Sequence: Synthetic oligonucleotide
acugacagcgugaacagguag

SEQ ID NO: 1163 - RNA, 21 nt, Artificial Sequence
  Description of Artificial Sequence: Synthetic oligonucleotide
ccaucggccugacucccaugc

SEQ ID NO: 1164 - RNA, 21 nt, Artificial Sequence
  Description of Artificial Sequence: Synthetic oligonucleotide
ccaucaccaucagcaccgggu

SEQ ID NO: 1165 - RNA, 21 nt, Artificial Sequence
  Description of Artificial Sequence: Synthetic oligonucleotide
ccugugugcuggugccccugc

SEQ ID NO: 1166 - RNA, 21 nt, Artificial Sequence
  Description of Artificial Sequence: Synthetic oligonucleotide
ugcagagaaagguggcucuau

SEQ ID NO: 1167 - RNA, 21 nt, Artificial Sequence
  Description of Artificial Sequence: Synthetic oligonucleotide
ucugcagaaauguuccccguu

SEQ ID NO: 1168 - RNA, 21 nt, Artificial Sequence
  Description of Artificial Sequence: Synthetic oligonucleotide
uaggaccuccaggaagauucu

SEQ ID NO: 1169 - RNA, 21 nt, Artificial Sequence
  Description of Artificial Sequence: Synthetic oligonucleotide
gcaacugaacaggaaauaacc

SEQ ID NO: 1170 - RNA, 21 nt, Artificial Sequence
  Description of Artificial Sequence: Synthetic oligonucleotide
guugcugccucuuuggguuug

SEQ ID NO: 1171 - RNA, 21 nt, Artificial Sequence
  Description of Artificial Sequence: Synthetic oligonucleotide
aagggaaugacaaaaccuauc

SEQ ID NO: 1172 - DNA, 21 bp, Artificial Sequence
  Description of Artificial Sequence: Synthetic oligonucleotide
tgatgtgagattttccacctg

SEQ ID NO: 1173 - DNA, 21 bp, Artificial Sequence
  Description of Artificial Sequence: Synthetic oligonucleotide
actgacagcgtgaacaggtag

SEQ ID NO: 1174 - DNA, 21 bp, Artificial Sequence
  Description of Artificial Sequence: Synthetic oligonucleotide
ccatcggcctgactcccatgc

SEQ ID NO: 1175 - DNA, 21 bp, Artificial Sequence
  Description of Artificial Sequence: Synthetic oligonucleotide
tgcagagaaaggtggctctat

SEQ ID NO: 1176 - DNA, 21 bp, Artificial Sequence
  Description of Artificial Sequence: Synthetic oligonucleotide
gcaactgaacaggaaataacc

SEQ ID NO: 1177 - DNA, 3993 bp, Artificial Sequence
  Description of Artificial Sequence: Synthetic polynucleotide
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcagctcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagtt

SEQ ID NO: 1178 - RNA, 21 nt, Artificial Sequence
  Description of Artificial Sequence: Synthetic oligonucleotide
aucuucuagguaugacaacga

SEQ ID NO: 1179 - DNA, 1431 bp, Artificial Sequence
  Description of Artificial Sequence: Synthetic polynucleotide
atgagccggagcgtggctctggccgtgctggccctgctgagcctgagcggcctcgaggctcggatcattcctcggcatctgcagctgggtggcggtggatccggtggcggtggatccggtggcggtggatccattcagcggacccccaaaatccaggtgtacagccggcaccctgctgaaaacggcaaaagcaattttctgaactgctatgtgagcggcttccaccccagcgatatcgaggtggacctgctgaaaaacggcgaacggatcgagaaagtggaacacagcgacctgagcttcagcaaggactggagcttttatctgctgtactataccgagttcacacccacagagaaggatgagtatgcctgccgggtgaaccacgtgaccctgagccagcctaaaatcgtgaagtgggatcgggatatgggtggcggtggatccggtggcggtggatccggtggcggtggatccggtggcggtggatccggcagccatagcatgcggtatttcagcgccgctgtgagccggcctggccggggcgaacctcggtttattgccatgggctatgtggacgatacccagttcgtgcggtttgatagcgatagcgcctgtccacggatggagcctcgggccccctgggtggagcaggaaggccccgaatattgggaagaggaaacacggaatacaaaggctcacgcccagacagatcggatgaatctgcagacactgcggggctactataaccagagcgaggctagcagccacaccctgcagtggatgattggctgtgacctgggcagcgatggccggctgctgcggggctacgagcagtacgcctatgatggcaaggactacctggctctgaacgaggacctgcggagctggacagccgctgacaccgccgctcagattagcaagcggaagtgtgaggctgccaacgtggctgaacagcggcgggcttatctggagggcacatgtgtggaatggctgcaccggtacctggagaatggcaaagagatgctgcagcgggccgaccccccaaaaacccacgtgacccaccatcccgtgttcgactacgaggctaccctgcggtgttgggccctgggcttttatcctgccgagatcattctgacatggcagcgggatggcgaggatcagacacaggatgtggagctggtggagacacggccagccggcgatggcacctttcagaaatgggccgctgtggtggtgcctagcggcgaagagcagcggtacacatgccatgtgcagcatgaaggcctgccagaacccctgatgctgcggtggaaacagagcagcctgcccacaatccctatcatgggcatcgtggctggcctggtggtgctggccgctgtggtgacaggcgccgctgtggccgctgtgctgtggcggaagaaaagcagcgac

SEQ ID NO: 1180 - protein, 477 aa, Artificial Sequence
  Description of Artificial Sequence: Synthetic polypeptide
MSRSVALAVLALLSLSGLEARIIPRHLQLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 1181 - DNA, 1500 bp, Artificial Sequence
  Description of Artificial Sequence: Synthetic polynucleotide
atgagccggagcgtggctctggccgtgctggccctgctgagcctgagcggcctcgaggctgtgatggcccctcggaccctgattctgggtggcggtggatccggtggcggtggatccggtggcggtggatccattcagcggacacccaaaatccaggtgtacagccggcatcccgccgaaaacggcaagagcaatttcctgaactgttacgtgagcggcttccaccccagcgacattgaagtggacctgctgaaaaacggcgagcggattgaaaaagtggaacacagcgacctgagctttagcaaagattggagcttttacctgctgtattacaccgaattcacccccaccgagaaggatgagtacgcctgccgggtgaaccatgtgaccctgagccagccaaaaatcgtgaagtgggatcgggatatgggtggcggtggatccggtggcggtggatccggtggcggtggatccggtggcggtggatccggcagccatagcctgaaatactttcacaccagcgtgagccggcctggccggggcgagccacggtttatcagcgtgggctatgtggacgatacccagtttgtgcggtttgacaatgacgctgccagccctcggatggtgccacgggctccctggatggaacaggagggcagcgaatattgggaccgggaaacccggagcgcccgggataccgcccagattttccgggtgaatctgcggaccctgcggggctactataaccagagcgaagctggcagccatacactgcagtggatgcacggctgtgagctgggcccagatggccggttcctgcggggctatgaacagtttgcctatgatggcaaagactatctgacactgaatgaagacctgcggagctggaccgccgtggacacagctgcccagattagcgagcagaagagcaatgatgccagcgaggccgagcatcagcgggcttacctggaggacacatgcgtggagtggctgcataaatatctggaaaaaggcaaggagacactgctgcatctggaacctccaaagacccacgtgacacaccatcctattagcgatcacgaggctaccctgcggtgctgggccctgggcttctaccccgccgagatcaccctgacctggcagcaggatggcgaaggccacacccaggataccgagctggtggaaacacggcctgccggcgacggcacattccagaagtgggctgccgtggtggtgcccagcggcgaagagcagcggtacacctgccatgtgcagcacgaaggcctgcctgaaccagtgaccctgcggtggaaaccagccagccagcccaccatccccatcgtgggcattatcgctggcctggtgctgctgggcagcgtggtgagcggcgccgtggtggccgctgtgatttggcggaagaaaagcagcggcggcaaaggcggcagctacagcaaggccgagtggagcgacagcgctcagggcagcgaaagccacagcctg

SEQ ID NO: 1182 - protein, 500 aa, Artificial Sequence
  Description of Artificial Sequence: Synthetic polypeptide
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 1183 - DNA, 915 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataac

SEQ ID NO: 1184 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 1185 - protein, 18 aa, Thosea asigna virus
EGRGSLLTCGDVEENPGP

SEQ ID NO: 1186 - protein, 19 aa, Teschovirus A
ATNFSLLKQAGDVEENPGP

SEQ ID NO: 1187 - protein, 20 aa, Equine rhinitis A virus
QCTNYALLKLAGDVESNPGP

SEQ ID NO: 1188 - protein, 22 aa, Foot-and-mouth disease virus
VKQTLNFDLLKLAGDVESNPGP

SEQ ID NO: 1189 - DNA, 54 bp, Thosea asigna virus
gagggcagaggaagtcttctaacatgcggtgacgtggaggagaatcctggcccg

SEQ ID NO: 1190 - DNA, 66 bp, Teschovirus A
ggaagcggagctactaacttcagcctgctgaagcaggctggagacgtggaggagaaccctggacct

SEQ ID NO: 1191 - DNA, 60 bp, Equine rhinitis A virus
cagtgtactaattatgctctcttgaaattggctggagatgttgagagcaaccctggacct

SEQ ID NO: 1192 - DNA, 66 bp, Foot-and-mouth disease virus
gtgaaacagactttgaattttgaccttctcaagttggcgggagacgtggagtccaaccctggacct

SEQ ID NO: 1193 - DNA, 574 bp, Artificial Sequence
  Description of Artificial Sequence: Synthetic polynucleotide
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 1194 - DNA, 555 bp, Artificial Sequence
  Description of Artificial Sequence: Synthetic polynucleotide
gaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactctttacatcttctaggtatgacaacgagttcggatatagcaatagagtggtcgatctgatggctcatatggctagcaaagag

SEQ ID NO: 1195 - DNA, 500 bp, Artificial Sequence
  Description of Artificial Sequence: Synthetic polynucleotide
atttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagt

SEQ ID NO: 1196 - DNA, 580 bp, Artificial Sequence
  Description of Artificial Sequence: Synthetic polynucleotide
ctgaccacagctctgcaagcagacttccatttacagtgaggaggtgagcattgcattgaacaaaagatggcgttttcacttggaattagttatctgaagctttaggattcctcagcaatatgattatgagacaagaaaggaagattcagaaatgagtctagttgaaggcagcaattcagagaagaagattcagttgttatcattgccgtcctgcttggtttatggcctggttcaggaccaaggagagaagtgtgaatacatgcctcttgagctatagaatgagacgctggagtcactaagatgattttttaaaagtattgttttataaacaaaaataagattgtgacaagggattccactattaatgttttcatgcctgtgccttaatctgactgggtatggtgagaattgtgcttgcagctttaaggtaagaattttaccatcttaatatgttaagaagtgccatttcagtctctcatctctactccaacttgtcttcttaggggctaaagtgcgggccgagatctacgaggccttcgagaatatctaccccatcctgaagggcttcagaaagaccacc

SEQ ID NO: 1197 - DNA, 500 bp, Artificial Sequence
  Description of Artificial Sequence: Synthetic polynucleotide
taggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttt

SEQ ID NO: 1198 - DNA, 2117 bp, Artificial Sequence
  Description of Artificial Sequence: Synthetic polynucleotide
gaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactctttacatcttctaggtatgacaacgagttcggatatagcaatagagtggtcgatctgatggctcatatggctagcaaagagggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagcggccgcgtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagt

SEQ ID NO: 1199 - DNA, 2142 bp, Artificial Sequence
  Description of Artificial Sequence: Synthetic polynucleotide
ctgaccacagctctgcaagcagacttccatttacagtgaggaggtgagcattgcattgaacaaaagatggcgttttcacttggaattagttatctgaagctttaggattcctcagcaatatgattatgagacaagaaaggaagattcagaaatgagtctagttgaaggcagcaattcagagaagaagattcagttgttatcattgccgtcctgcttggtttatggcctggttcaggaccaaggagagaagtgtgaatacatgcctcttgagctatagaatgagacgctggagtcactaagatgattttttaaaagtattgttttataaacaaaaataagattgtgacaagggattccactattaatgttttcatgcctgtgccttaatctgactgggtatggtgagaattgtgcttgcagctttaaggtaagaattttaccatcttaatatgttaagaagtgccatttcagtctctcatctctactccaacttgtcttcttaggggctaaagtgcgggccgagatctacgaggccttcgagaatatctaccccatcctgaagggcttcagaaagaccaccggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagcggccgcgtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttt

SEQ ID NO: 1200 - RNA, 21 nt, Artificial Sequence
  Description of Artificial Sequence: Synthetic oligonucleotide
cugguauguggcuggggccag

SEQ ID NO: 1201 - RNA, 21 nt, Artificial Sequence
  Description of Artificial Sequence: Synthetic oligonucleotide
ugagccagccaccagagggcg

SEQ ID NO: 1202 - RNA, 21 nt, Artificial Sequence
  Description of Artificial Sequence: Synthetic oligonucleotide
gcuacagcaacaggguggugg

SEQ ID NO: 1203 - RNA, 21 nt, Artificial Sequence
  Description of Artificial Sequence: Synthetic oligonucleotide
ccauaauuuccuuucaaggug

SEQ ID NO: 1204 - RNA, 21 nt, Artificial Sequence
  Description of Artificial Sequence: Synthetic oligonucleotide
cuuucaagguggggagggagg

SEQ ID NO: 1205 - RNA, 21 nt, Artificial Sequence
  Description of Artificial Sequence: Synthetic oligonucleotide
aagguggggagggagguagag

SEQ ID NO: 1206 - RNA, 21 nt, Artificial Sequence
  Description of Artificial Sequence: Synthetic oligonucleotide
gcagaccacaguccaugccau

SEQ ID NO: 1207 - RNA, 21 nt, Artificial Sequence
  Description of Artificial Sequence: Synthetic oligonucleotide
cagaccacaguccaugccauc

SEQ ID NO: 1208 - RNA, 21 nt, Artificial Sequence
  Description of Artificial Sequence: Synthetic oligonucleotide
ccggaggggccauccacaguc

SEQ ID NO: 1209 - RNA, 21 nt, Artificial Sequence
  Description of Artificial Sequence: Synthetic oligonucleotide
uagacggcaggucagguccac

SEQ ID NO: 1210 - RNA, 21 nt, Artificial Sequence
  Description of Artificial Sequence: Synthetic oligonucleotide
cuagacggcaggucaggucca

SEQ ID NO: 1211 - RNA, 21 nt, Artificial Sequence
  Description of Artificial Sequence: Synthetic oligonucleotide
ucuagacggcaggucaggucc

SEQ ID NO: 1212 - RNA, 21 nt, Artificial Sequence
  Description of Artificial Sequence: Synthetic oligonucleotide
gcagguuuuucuagacggcag

SEQ ID NO: 1213 - RNA, 21 nt, Artificial Sequence
  Description of Artificial Sequence: Synthetic oligonucleotide
ucaagcucauuuccugguaug

SEQ ID NO: 1214 - RNA, 21 nt, Artificial Sequence
  Description of Artificial Sequence: Synthetic oligonucleotide
agagccagucucuggccccag

SEQ ID NO: 1215 - RNA, 21 nt, Artificial Sequence
  Description of Artificial Sequence: Synthetic oligonucleotide
aagagccagucucuggcccca

SEQ ID NO: 1216 - RNA, 21 nt, Artificial Sequence
  Description of Artificial Sequence: Synthetic oligonucleotide
uaagagccagucucuggcccc

SEQ ID NO: 1217 - RNA, 21 nt, Artificial Sequence
  Description of Artificial Sequence: Synthetic oligonucleotide
cugagccagccaccagagggc

SEQ ID NO: 1218 - RNA, 21 nt, Artificial Sequence
  Description of Artificial Sequence: Synthetic oligonucleotide
ucugagccagccaccagaggg

SEQ ID NO: 1219 - RNA, 21 nt, Artificial Sequence
  Description of Artificial Sequence: Synthetic oligonucleotide
caucuucuagguaugacaacg

SEQ ID NO: 1220 - RNA, 21 nt, Artificial Sequence
  Description of Artificial Sequence: Synthetic oligonucleotide
aaaugcuucauaaauuucugc

SEQ ID NO: 1221 - RNA, 21 nt, Artificial Sequence
  Description of Artificial Sequence: Synthetic oligonucleotide
ugcucugacuuuagcaccuaa

SEQ ID NO: 1222 - RNA, 21 nt, Artificial Sequence
  Description of Artificial Sequence: Synthetic oligonucleotide
aaaacaucuacccuauucuaa

SEQ ID NO: 1223 - RNA, 21 nt, Artificial Sequence
  Description of Artificial Sequence: Synthetic oligonucleotide
ccccucugcuucgucuuucuc

SEQ ID NO: 1224 - RNA, 21 nt, Artificial Sequence
  Description of Artificial Sequence: Synthetic oligonucleotide
uccacccccgggagaccacga

SEQ ID NO: 1225 - RNA, 21 nt, Artificial Sequence
  Description of Artificial Sequence: Synthetic oligonucleotide
augugccuguucucaaccucu

SEQ ID NO: 1226 - RNA, 21 nt, Artificial Sequence
  Description of Artificial Sequence: Synthetic oligonucleotide
caguaugagggcaccuacaag

SEQ ID NO: 1227 - RNA, 21 nt, Artificial Sequence
  Description of Artificial Sequence: Synthetic oligonucleotide
ccgccuuaaauccacagcaua

SEQ ID NO: 1228 - RNA, 21 nt, Artificial Sequence
  Description of Artificial Sequence: Synthetic oligonucleotide
uaaccaagugcucuguaguuu

SEQ ID NO: 1229 - RNA, 21 nt, Artificial Sequence
  Description of Artificial Sequence: Synthetic oligonucleotide
gaccucuccaguguguuaaug

SEQ ID NO: 1230 - DNA, 1485 bp, Artificial Sequence
  Description of Artificial Sequence: Synthetic polynucleotide
atgagccggagcgtggctctggccgtgctggccctgctgagcctgagcggcctcgaggctgtgatggcccctcggaccctgattctgggtggcggtggatccggtggcggtggatccggtggcggtggatccattcagcggacacccaaaatccaggtgtacagccggcatcccgccgaaaacggcaagagcaatttcctgaactgttacgtgagcggcttccaccccagcgacattgaagtggacctgctgaaaaacggcgagcggattgaaaaagtggaacacagcgacctgagctttagcaaagattggagcttttacctgctgtattacaccgaattcacccccaccgagaaggatgagtacgcctgccgggtgaaccatgtgaccctgagccagccaaaaatcgtgaagtgggatcgggatatgggtggcggtggatccggtggcggtggatccggtggcggtggatccggcagccatagcctgaaatactttcacaccagcgtgagccggcctggccggggcgagccacggtttatcagcgtgggctatgtggacgatacccagtttgtgcggtttgacaatgacgctgccagccctcggatggtgccacgggctccctggatggaacaggagggcagcgaatattgggaccgggaaacccggagcgcccgggataccgcccagattttccgggtgaatctgcggaccctgcggggctactataaccagagcgaagctggcagccatacactgcagtggatgcacggctgtgagctgggcccagatggccggttcctgcggggctatgaacagtttgcctatgatggcaaagactatctgacactgaatgaagacctgcggagctggaccgccgtggacacagctgcccagattagcgagcagaagagcaatgatgccagcgaggccgagcatcagcgggcttacctggaggacacatgcgtggagtggctgcataaatatctggaaaaaggcaaggagacactgctgcatctggaacctccaaagacccacgtgacacaccatcctattagcgatcacgaggctaccctgcggtgctgggccctgggcttctaccccgccgagatcaccctgacctggcagcaggatggcgaaggccacacccaggataccgagctggtggaaacacggcctgccggcgacggcacattccagaagtgggctgccgtggtggtgcccagcggcgaagagcagcggtacacctgccatgtgcagcacgaaggcctgcctgaaccagtgaccctgcggtggaaaccagccagccagcccaccatccccatcgtgggcattatcgctggcctggtgctgctgggcagcgtggtgagcggcgccgtggtggccgctgtgatttggcggaagaaaagcagcggcggcaaaggcggcagctacagcaaggccgagtggagcgacagcgctcagggcagcgaaagccacagcctg

SEQ ID NO: 1231 - protein, 495 aa, Artificial Sequence
  Description of Artificial Sequence: Synthetic polypeptide
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 1232 - DNA, 1467 bp, Artificial Sequence
  Description of Artificial Sequence: Synthetic polynucleotide
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 1233 - protein, 489 aa, Artificial Sequence
  Description of Artificial Sequence: Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1234 - protein, 9 aa, Homo sapiens
RIIPRHLQL

SEQ ID NO: 1235 - protein, 9 aa, Homo sapiens
VMAPRTLFL

SEQ ID NO: 1236 - protein, 9 aa, Homo sapiens
VMAPRTLIL

SEQ ID NO: 1237 - protein, 9 aa, Homo sapiens
VMAPRTVLL

SEQ ID NO: 1238 - protein, 9 aa, Homo sapiens
VMAPRTLVL

SEQ ID NO: 1239 - skipped

SEQ ID NO: 1240 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 1241 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 1242 - protein, 339 aa, Homo sapiens
MMVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 1243 - protein, 500 aa, Artificial Sequence
  Description of Artificial Sequence: Synthetic polypeptide
MSRSVALAVLALLSLSGLEAVMAPRTLVLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 1244 - protein, 500 aa, Artificial Sequence
  Description of Artificial Sequence: Synthetic polypeptide
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 1245 - protein, 500 aa, Artificial Sequence
  Description of Artificial Sequence: Synthetic polypeptide
MSRSVALAVLALLSLSGLEAVMAPRTVLLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 1246 - protein, 500 aa, Artificial Sequence
  Description of Artificial Sequence: Synthetic polypeptide
MSRSVALAVLALLSLSGLEARIIPRHLQLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 1247 - protein, 15 aa, Artificial Sequence
  Description of Artificial Sequence: Synthetic peptide
GGGGSGGGGSGGGGS

SEQ ID NO: 1248 - protein, 20 aa, Artificial Sequence
  Description of Artificial Sequence: Synthetic peptide
GGGGSGGGGSGGGGSGGGGS

SEQ ID NO: 1249 - protein, 20 aa, Homo sapiens
MSRSVALAVLALLSLSGLEA

SEQ ID NO: 1250 - protein, 99 aa, Homo sapiens [sequence omitted]

SEQ ID NO: 1251 - protein, 337 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 1252 - protein, 314 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD